SEQUENCE LISTING (56 sequences)

SEQ ID NO: 1 - protein, 117 aa, synthetic construct
  source 1..117: note = 101A6 VH amino acid sequence
EVKLVESGGGLVQPGGSLSLSCAASGFTFTDYYLSWVRQPPGKALEWLALIRNKANGYTTEYSASVKGRFTISRDSSQSILYLQMDALRAEDSATYYCARDTLAAYWGQGTSVTVSS

SEQ ID NO: 2 - protein, 107 aa, synthetic construct
  source 1..107: note = 101A6 VL amino acid sequence
DIVMTQSHKFMSTSVGDRVSFTCKASQDVGSAVIWYQQKPGQSPKLLIYWASTRHTGVPDRFTGSGSGTDFTLTISNVQSEDLADYFCQQYSSYPLTFGAGTKLEIK

SEQ ID NO: 3 - protein, 117 aa, synthetic construct
  source 1..117: note = 101A6HZ/101A6HZm VH amino acid sequence
EVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSS

SEQ ID NO: 4 - protein, 107 aa, synthetic construct
  source 1..107: note = 101A6HZ/101A6HZm VL amino acid sequence
DIVMTQSPDSLAVSLGERATINCKASQDVGSAVIWYQQKPGQSPKLLIYWASTRHTGVPDRFSGSGSGTDFTLTISSLQAEDVAVYYCQQYSSYPLTFGQGTKLEIK

SEQ ID NO: 5 - protein, 120 aa, synthetic construct
  source 1..120: note = 64A10 VH amino acid sequence
QVQLKESGPVLVAPSQSLSITCTVSGFSLTSYGVYWVRQPPGKGPEWLGVIWTSGNTNYNSALMSRLSISKDNSKSQVFLKMNSLQTDDTAMYYCVKHDGGNYVGVMNYWGQGTSVTVSS

SEQ ID NO: 6 - protein, 107 aa, synthetic construct
  source 1..107: note = 64A10 VL amino acid sequence
DIQMTQSPATLSVTPGDRVSLSCRASQSISDYLHWYQQKSQESPRLLIKYASQSISGIPSRFSGSGSGSDFTLSINSVEPEDVGVYYCQNGHSFPPTFGGGTKLEIK

SEQ ID NO: 7 - protein, 120 aa, synthetic construct
  source 1..120: note = 64A10HZ VH amino acid sequence
QVQLQESGPGLVKPSETLSLTCTVSGFSLTSYGVYWVRQPPGKGPEWLGVIWTSGNTNYNPSLKSRLTISKDNSKNQVSLKLSSVTAADTAVYYCVKHDGGNYVGVMNYWGQGTLVTVSS

SEQ ID NO: 8 - protein, 107 aa, synthetic construct
  source 1..107: note = 64A10HZ VL amino acid sequence
EIVMTQSPDFQSVTPKEKVTITCRASQSISDYLHWYQQKPDQSPKLLIKYASQSISGVPSRFSGSGSGSDFTLTINSLEAEDAATYYCQNGHSFPPTFGQGTKLEIK

SEQ ID NO: 9 - protein, 120 aa, synthetic construct
  source 1..120: note = 64A10HZ05 VH amino acid sequence
QVQLQESGPGLVKPSETLSLTCTVSGFSLTSYGVYWVRQPPGKGLEWLGVIWTSGNTNYNPSLKSRLTISKDNSKNQVSLKLSSVTAADTAVYYCVKHDGGNYVGVMNYWGQGTLVTVSS

SEQ ID NO: 10 - protein, 107 aa, synthetic construct
  source 1..107: note = 64A10HZ05 VL amino acid sequence
DIQMTQSPSSLSASVGDRVTITCRASQSISDYLHWYQQKPGKAPKLLIKYASQSISGVPSRFSGSGSGSDFTLTISSLQPEDFATYYCQNGHSFPPTFGGGTKVEIK

SEQ ID NO: 11 - protein, 7 aa, synthetic construct
  source 1..7: note = 101A6/101A6HZ/101A6HZm Chothia CDR H1
GFTFTDY

SEQ ID NO: 12 - protein, 8 aa, synthetic construct
  source 1..8: note = 101A6/101A6HZ/101A6HZm Chothia CDR H2
RNKANGYT

SEQ ID NO: 13 - protein, 6 aa, synthetic construct
  source 1..6: note = 101A6/101A6HZ/101A6HZm Chothia/kabat/AbM CDR H3
DTLAAY

SEQ ID NO: 14 - protein, 11 aa, synthetic construct
  source 1..11: note = 101A6/101A6HZ/101A6HZm Chothia/Kabat/AbM CDR L1
KASQDVGSAVI

SEQ ID NO: 15 - protein, 7 aa, synthetic construct
  source 1..7: note = 101A6/101A6HZ/101A6HZm Chothia/Kabat/AbM CDR L2
WASTRHT

SEQ ID NO: 16 - protein, 9 aa, synthetic construct
  source 1..9: note = 101A6/101A6HZ/101A6HZm Chothia/Kabat/IMGT/AbM CDR L3
QQYSSYPLT

SEQ ID NO: 17 - protein, 5 aa, synthetic construct
  source 1..5: note = 101A6/101A6HZ/101A6HZm Kabat CDR H1
DYYLS

SEQ ID NO: 18 - protein, 19 aa, synthetic construct
  source 1..19: note = 101A6HZ/101A6HZm Kabat CDR H2
LIRNKANGYTTEYAASVKG

SEQ ID NO: 19 - protein, 19 aa, synthetic construct
  source 1..19: note = 101A6 Kabat CDR H2
LIRNKANGYTTEYSASVKG

SEQ ID NO: 20 - protein, 8 aa, synthetic construct
  source 1..8: note = 101A6/101A6HZ/101A6HZm IMGT CDR H1
GFTFTDYY

SEQ ID NO: 21 - protein, 10 aa, synthetic construct
  source 1..10: note = 101A6/101A6HZ/101A6HZm IMGT CDR H2
IRNKANGYTT

SEQ ID NO: 22 - protein, 8 aa, synthetic construct
  source 1..8: note = 101A6/101A6HZ/101A6HZm IMGT CDR H3
ARDTLAAY

SEQ ID NO: 23 - protein, 6 aa, synthetic construct
  source 1..6: note = 101A6/101A6HZ/101A6HZm IMGT CDR L1
QDVGSA

SEQ ID NO: 24 - skipped

SEQ ID NO: 25 - protein, 10 aa, synthetic construct
  source 1..10: note = 101A6/101A6HZ/101A6HZm AbM CDR H1
GFTFTDYYLS

SEQ ID NO: 26 - protein, 12 aa, synthetic construct
  source 1..12: note = 101A6/101A6HZ/101A6HZm AbM CDR H2
LIRNKANGYTTE

SEQ ID NO: 27 - protein, 7 aa, synthetic construct
  source 1..7: note = 64A10/64A10HZ/64A10HZ05 Chothia CDR H1
GFSLTSY

SEQ ID NO: 28 - protein, 5 aa, synthetic construct
  source 1..5: note = 64A10/64A10HZ/64A10HZ05 Chothia CDR H2
WTSGN

SEQ ID NO: 29 - protein, 12 aa, synthetic construct
  source 1..12: note = 64A10/64A10HZ/64A10HZ05 Chothia/Kabat/AbM CDR H3
HDGGNYVGVMNY

SEQ ID NO: 30 - protein, 11 aa, synthetic construct
  source 1..11: note = 64A10/64A10HZ/64A10HZ05 Chothia/Kabat/AbM CDR L1
RASQSISDYLH

SEQ ID NO: 31 - protein, 7 aa, synthetic construct
  source 1..7: note = 64A10/64A10HZ/64A10HZ05 Chothia/Kabat/AbM CDR L2
YASQSIS

SEQ ID NO: 32 - protein, 9 aa, synthetic construct
  source 1..9: note = 64A10/64A10HZ/64A10HZ05 Chothia/Kabat/IMGT/AbM CDR L3
QNGHSFPPT

SEQ ID NO: 33 - protein, 5 aa, synthetic construct
  source 1..5: note = 64A10/64A10HZ/64A10HZ05 Kabat CDR H1
SYGVY

SEQ ID NO: 34 - protein, 16 aa, synthetic construct
  source 1..16: note = 64A10HZ/64A10HZ05 Kabat CDR H2
VIWTSGNTNYNPSLKS

SEQ ID NO: 35 - protein, 16 aa, synthetic construct
  source 1..16: note = 64A10 Kabat CDR H2
VIWTSGNTNYNSALMS

SEQ ID NO: 36 - protein, 8 aa, synthetic construct
  source 1..8: note = 64A10/64A10HZ/64A10HZ05 IMGT CDR H1
GFSLTSYG

SEQ ID NO: 37 - protein, 7 aa, synthetic construct
  source 1..7: note = 64A10/64A10HZ/64A10HZ05 IMGT CDR H2
IWTSGNT

SEQ ID NO: 38 - protein, 14 aa, synthetic construct
  source 1..14: note = 64A10/64A10HZ/64A10HZ05 IMGT CDR H3
VKHDGGNYVGVMNY

SEQ ID NO: 39 - protein, 6 aa, synthetic construct
  source 1..6: note = 64A10/64A10HZ/64A10HZ05 IMGT CDR L1
QSISDY

SEQ ID NO: 40 - skipped

SEQ ID NO: 41 - protein, 10 aa, synthetic construct
  source 1..10: note = 64A10/64A10HZ/64A10HZ05 AbM CDR H1
GFSLTSYGVY

SEQ ID NO: 42 - protein, 9 aa, synthetic construct
  source 1..9: note = 64A10/64A10HZ/64A10HZ05 AbM CDR H2
VIWTSGNTN

SEQ ID NO: 43 - protein, 330 aa, synthetic construct [sequence omitted]

SEQ ID NO: 44 - protein, 107 aa, synthetic construct [sequence omitted]

SEQ ID NO: 45 - protein, 330 aa, synthetic construct
  source 1..330: note = Mutant human heavy chain constant region IgG1
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 447 aa, synthetic construct
  source 1..447: note = 101A6HZm HC
EVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 214 aa, synthetic construct
  source 1..214: note = 101A6HZ/101A6HZm LC
DIVMTQSPDSLAVSLGERATINCKASQDVGSAVIWYQQKPGQSPKLLIYWASTRHTGVPDRFSGSGSGTDFTLTISSLQAEDVAVYYCQQYSSYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 4 aa, synthetic construct
  source 1..4: note = Linker
GGFG

SEQ ID NO: 49 - protein, 4 aa, synthetic construct
  source 1..4: note = Linker
GFLG

SEQ ID NO: 50 - protein, 4 aa, synthetic construct
  source 1..4: note = Linker
GGVA

SEQ ID NO: 51 - protein, 5 aa, synthetic construct
  source 1..5: note = Linker
GGGGG

SEQ ID NO: 52 - protein, 446 aa, synthetic construct
  source 1..446: note = 101A6HZm heavy chain amino acid sequence (Pyroglutamate or Pyroglutamic acid at N-terminus; no C-terminal lysine)
  MOD_RES 1: note = pyroglutamate or pyroglutamic acid
XVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 446 aa, synthetic construct
  source 1..446: note = 101A6HZm heavy chain amino acid sequence (no C-terminal lysine)
EVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 54 - protein, 447 aa, synthetic construct
  source 1..447: note = 101A6HZm heavy chain amino acid sequence (Pyroglutamate or Pyroglutamic acid at N-terminus)
  MOD_RES 1: note = pyroglutamate or pyroglutamic acid
XVQLVESGGGLVQPGGSLRLSCAASGFTFTDYYLSWVRQAPGKGLEWLALIRNKANGYTTEYAASVKGRFTISRDSSKNSLYLQMNSLKTEDTAVYYCARDTLAAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - protein, 5 aa, synthetic construct
  source 1..5: note = Linker
GGGGS

SEQ ID NO: 56 - protein, 20 aa, synthetic construct
  source 1..20: note = Linker
GGGGSGGGGSGGGGSGGGGS

(19) United States
(12) Patent Application Publication
(10) Pub. No.: US 2026/0355334 A1
(43) Pub. Date: Nov. 12, 2026

(54) ANTIBODY-DRUG CONJUGATE BINDING TO HUMAN PTK7 AND METHOD FOR PREPARATION AND USE THEREOF

(71) Applicant: SICHUAN KELUN-BIOTECH BIOPHARMACEUTICAL CO., LTD., Chengdu, Sichuan (CN)

(72) Inventors: Hu LONG, Chengdu, Sichuan (CN); Shuntao LUO, Chengdu, Sichuan (CN); Qiang TIAN, Chengdu, Sichuan (CN); Xiaoxi YUAN, Chengdu, Sichuan (CN); Yuehua ZHOU, Chengdu, Sichuan (CN); Qian CHEN, Chengdu, Sichuan (CN); Ruibin HU, Chengdu, Sichuan (CN); Liangbo LUO, Chengdu, Sichuan (CN); Yitao ZHANG, Chengdu, Sichuan (CN); Ying WU, Chengdu, Sichuan (CN); Hongmei SONG, Chengdu, Sichuan (CN); Miao TAN, Chengdu, Sichuan (CN); Xiangyang TAN; Junyou GE, Chengdu, Sichuan (CN)

(73) Assignee: SICHUAN KELUN-BIOTECH BIOPHARMACEUTICAL CO., LTD., Chengdu, Sichuan (CN)

(21) Appl. No.: 19/114,048

(22) PCT Filed: Oct. 12, 2023
(86) PCT No.: PCT/CN2023/124279

§ 371 Date: Mar. 21, 2025

(30) Foreign Application Priority Data
Oct. 14, 2022 (CN) 202211263034.0
Sep. 28, 2023 (CN) 202311284321.4

(51) Int. Cl. A61K 47/68; A61P 35/00; C07K 16/30

(52) U.S. Cl. CPC A61K 47/68037; A61K 47/6849; A61K 47/6889; A61P 35/00; C07K 16/30

(57) ABSTRACT

The present application relates to an antibody-drug conjugate and a method for preparation and use thereof, and specifically relates to an antibody-drug conjugate for treating PTK7-positive cancer, including a PTK7 antibody, and drug-linker molecules conjugated to the antibody. In some embodiments, the antibody is humanized, has excellent binding activity to PTK7-positive cells, and can efficiently deliver drugs to PTK7-positive cells. In some embodiments, the drug includes a DNA topoisomerase inhibitor. The antibody-drug conjugate of the present application has a better drug-antibody conjugation ratio, and has a good targeted killing effect on lung cancer, breast cancer, epidermal cancer, ovarian cancer, esophageal cancer, and the like. Therefore, the present application further provides a method for preparation of the antibody-drug conjugate and use thereof in treatment of PTK7-positive cancer.

### CROSS REFERENCE TO RELATED APPLICATIONS

This application claims the benefit of Chinese Application Nos. 202311284321.4, filed Sep. 28, 2023 and 202211263034.0, filed Oct. 14, 2022, the disclosures of each of which are incorporated herein by reference in their entirety.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “14463-051-228_SEQ_LISTING.xml”, was created on Sep. 14, 2023, and is 63,238 bytes in size.

### BACKGROUND OF THE INVENTION

### (1) Field of the Invention

The present application relates to the field of targeted therapy, and specifically relates to an antibody-drug conjugate for treating PTK7-positive cancer. Specifically, the antibody-drug conjugate comprises a PTK7 antibody, and drug-linker molecules conjugated to the antibody. In some embodiments, the antibody is humanized, has excellent binding activity to PTK7-positive cells, and can efficiently deliver drugs to PTK7-positive cells. In some embodiments, the drug comprises a DNA topoisomerase inhibitor. The antibody-drug conjugate of the present application has excellent drug-antibody conjugation ratio, and has good targeted killing effect on lung cancer, breast cancer, epidermal cancer, ovarian cancer and esophageal cancer. Therefore, the present application further provides a method for preparing the antibody-drug conjugate and use thereof in treating PTK7 positive cancer.

### (2) Description of Related Art

Cancer is one of the leading causes of death in modern times. It is a class of diseases caused by malignant transformation of healthy cells, caused by genetic changes, such as chromosomal translocations, mutations in tumor suppressor genes and growth factor receptors, leading to malignant proliferation of cells. Defective apoptosis, or programmed cell death, further promotes malignant transformation of cells that lead to cancer. According to the latest assessment of the International Agency for Research on Cancer (IARC) under the World Health Organization (WHO), there were 19.29 million new cancer cases worldwide in 2020, including 10.06 million males and 9.23 million females. In 2020, there were 9.96 million cancer deaths worldwide, including 5.53 million males and 4.43 million females. In this century, cancer is predicted to surpass cardiovascular disease as the leading cause of premature death in most countries.

PTK7 (protein tyrosine kinase 7) belongs to receptor tyrosine kinase family, and is lack of kinase activity due to the mutation of the kinase domain. PTK7 protein is composed of 7 extracellular immunoglobulin domains, a transmembrane domain and an intracellular tyrosine kinase domain, and its ligand is unknown. The extracellular segment of PTK7 can be cleaved by ADAM and MT1-MMP proteases to produce soluble fragments, and the concentration of free PTK7 in the serum of healthy people is about 12.4±3.3 ng/ml; and the concentration in tumor patients is higher, about 24.6±3.8 ng/ml.

PTK7 is expressed in a variety of solid tumors: it is reported that it is highly expressed in 47.4% of NSCLC, 45.1% of ovarian cancer, 28.6% of TNBC, and 60% of esophageal cancer, and the studies have shown that PTK7 expression promotes the growth of tumor cells, and knocking out the PTK7 gene reduces the tumor burden in mice. High expression of PTK7 was positively correlated with disease staging and lymph node metastasis, and negatively correlated with survival rate. At present, there have been studies on PTK7-targeted antibody-drug conjugates, CAR-T and other treatment methods. In preclinical studies, tumor cell lines with high expression of PTK7 would be efficiently killed. It showed strong tumor suppressive activity in human-derived tumor cell derived xenograft (CDX) and human-derived tumor xenograft (PDX) models. Cofetuzumab Pelidotin, an ADC drug targeting PTK7 and developed by Pfizer, has shown good safety and preliminary efficacy in clinical stage I.

ADC drugs are composed of antibodies, bioactive molecules and linkers. Biologically active molecules are covalently conjugated to the antibodies through the linkers; the antibodies (for example monoclonal antibodies) can specifically recognize specific targets on the surfaces of tumor cells, and then guide ADCs to the surfaces of cancer cells, and enable ADCs to enter the cancer cells through endocytosis; and then the bioactive molecules are released in the cancer cells to kill the cancer cells without damaging the normal tissue cells as much as possible. Developing antibodies with better targeting, hydrophilicity, and endocytic activity for conjugating cytotoxic small molecules to prepare ADC drugs with a larger therapeutic window will provide patients with better treatment options.

### BRIEF SUMMARY OF THE INVENTION

The present application relates to an antibody-drug conjugate for treating PTK7-positive cancers, and exemplarily discloses an antibody-drug conjugate using humanized antibodies 101A6HZ, 101A6HZm and 64A10HZ as targeting portions and having a structure represented by a general formula Ab-[M-L-E-D]ₓ. The results show that the conjugate has a better drug-antibody conjugation ratio, and the conjugate has excellent binding activity to PTK7-positive cells, and has a good target killing effect on PTK7-positive cells, for example lung cancer, breast cancer, epidermal cancer, ovarian cancer and esophageal cancer cells. Therefore, the present application provides an antibody-drug conjugate for treating cancers with high expression of PTK7, a pharmaceutical composition comprising the antibody-drug conjugate, and use thereof in treatment of cancers with high expression of PTK7.

### Antibody-Drug Conjugate

In one aspect, the present application provides an antibody-drug conjugate having a structure shown as a formula Ab-[M-L-E-D]ₓ, where:
Ab is an antibody or antigen-binding fragment thereof that specifically binds to human tyrosine kinase 7 (PTK7);
M is a linker site linked to the antibody or antigen-binding fragment thereof;
L is a linker between M and E;
E is a structural fragment linking L and D;
D is a cytotoxic drug fragment; and
x is selected from 1 to 10.

In some embodiments, the antibody or antigen-binding fragment thereof comprises:
(1) the following heavy chain variable regions (VH) and/or light chain variable regions (VL), wherein CDRs are defined according to a Chothia numbering system:
(1a) a heavy chain variable region (VH) comprising the following 3 CDRs: CDR-H1 having a sequence shown as SEQ ID NO: 11 or a variant thereof, CDR-H2 having a sequence shown as SEQ ID NO: 12 or a variant thereof, and CDR-H3 having a sequence shown as SEQ ID NO: 13 or a variant thereof; and/or, a light chain variable region (VL) comprising the following 3 CDRs: CDR-L1 having a sequence shown as SEQ ID NO: 14 or a variant thereof, CDR-L2 having a sequence shown as SEQ ID NO: 15 or a variant thereof, and CDR-L3 having a sequence shown as SEQ ID NO: 16 or a variant thereof; or,
(1b) a heavy chain variable region (VH) comprising the following 3 CDRs: CDR-H1 having a sequence shown as SEQ ID NO: 27 or a variant thereof, CDR-H2 having a sequence shown as SEQ ID NO: 28 or a variant thereof, CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof, CDR-L2 having a sequence shown as SEQ ID NO: 31 or a variant thereof, and CDR-L3 having a sequence shown as SEQ ID NO: 32 or a variant thereof;
where the variants in any one of (1a) and (1b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example replacement, deletion or addition of 1, 2 or 3 amino acids) compared with the sequence from which the variants are derived; preferably, the replacement is conservative replacement;
or,
(2) the following heavy chain variable region (VH) and/or light chain variable region (VL), wherein the CDRs are defined according to the Kabat numbering system:

(2a) a heavy chain variable region (VH) comprising the following 3 CDRs: CDR-H1 having a sequence shown as SEQ ID NO: 17 or a variant thereof, CDR-H2 having a sequence shown as SEQ ID NO: 18 or 19 or a variant thereof, CDR-H3 having a sequence shown as SEQ ID NO: 13 or a variant thereof; and/or, a light chain variable region (VL) comprising the following 3 CDRs: CDR-L1 having a sequence shown as SEQ ID NO: 14 or a variant thereof, CDR-L2 having a sequence shown as SEQ ID NO: 15 or a variant thereof, and CDR-L3 having a sequence shown as SEQ ID NO: 16 or a variant thereof; or,
(2b) a heavy chain variable region (VH) comprising the following 3 CDRs: CDR-H1 having a sequence shown as SEQ ID NO: 33 or a variant thereof, CDR-H2 having a sequence shown as SEQ ID NO: 34 or 35 or a variant thereof, and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof, CDR-L2 having a sequence shown as SEQ ID NO: 31 or a variant thereof, and CDR-L3 having a sequence shown as SEQ ID NO: 32 or a variant thereof;

where the variants in any one of (2a) and (2b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement;
or,
(3) the following heavy chain variable regions (VH) and/or light chain variable regions (VL), wherein the CDRs are defined according to the IMGT numbering system:

(3a) a heavy chain variable region (VH) comprising the following 3 CDRs: CDR-H1 having a sequence shown as SEQ ID NO: 20 or a variant thereof, CDR-H2 having a sequence shown as SEQ ID NO: 21 or a variant thereof, and CDR-H3 having a sequence shown as SEQ ID NO: 22 or a variant thereof; and/or, a light chain variable region (VL) comprising the following 3 CDRs: CDR-L1 having a sequence shown as SEQ ID NO: 23 or a variant thereof, CDR-L2 having a sequence shown as SEQ ID NO: 24 or a variant thereof, and CDR-L3 having a sequence shown as SEQ ID NO: 16 or a variant thereof; or,
(3b) a heavy chain variable region (VH) comprising the following 3 CDRs: CDR-H1 having a sequence shown as SEQ ID NO: 36 or a variant thereof, CDR-H2 having a sequence shown as SEQ ID NO: 37 or a variant thereof, and CDR-H3 having a sequence shown as SEQ ID NO: 38 or a variant thereof; and/or, a light chain variable region (VL) comprising the following 3 CDRs: CDR-L1 having a sequence shown as SEQ ID NO: 39 or a variant thereof, CDR-L2 having a sequence shown as SEQ ID NO: 40 or a variant thereof, and CDR-L3 having a sequence shown as SEQ ID NO: 32 or a variant thereof;
where the variants in any one of (3a) and (3b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement;
or,
(4) the following heavy chain variable regions (VH) and/or light chain variable regions (VL), wherein the CDRs are defined according to the AbM numbering system:

(4a) a heavy chain variable region (VH) comprising the following 3 CDRs: CDR-H1 having a sequence shown as SEQ ID NO: 25 or a variant thereof, CDR-H2 having a sequence shown as SEQ ID NO: 26 or a variant thereof, and CDR-H3 having a sequence shown as SEQ ID NO: 13 or a variant thereof; and/or, a light chain variable region (VL) comprising the following 3 CDRs: CDR-L1 having a sequence shown as SEQ ID NO: 14 or a variant thereof, CDR-L2 having a sequence shown as SEQ ID NO: 15 or a variant thereof, and CDR-L3 having a sequence shown as SEQ ID NO: 16 or a variant thereof; or,
(4b) a heavy chain variable region (VH) comprising the following 3 CDRs: CDR-H1 having a sequence shown as SEQ ID NO: 41 or a variant thereof, CDR-H2 having a sequence shown as SEQ ID NO: 42 or a variant thereof, and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof, CDR-L2 having a sequence shown as SEQ ID NO: 31 or a variant thereof, and CDR-L3 having a sequence shown as SEQ ID NO: 32 or a variant thereof;
where the variants in any one of (4a) and (4b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement.

In some embodiments, the antibody or antigen-binding fragment thereof comprises:
(a) a VH shown as SEQ ID NO: 1 or a variant thereof, and/or, a VL shown as SEQ ID NO: 2 or a variant thereof;
(b) a VH shown as SEQ ID NO: 3 or a variant thereof, and/or, a VL shown as SEQ ID NO: 4 or a variant thereof; or
(c) a VH shown as SEQ ID NO: 5 or a variant thereof, and/or, a VL shown as SEQ ID NO: 6 or a variant thereof;
(d) a VH shown as SEQ ID NO: 7 or a variant thereof, and/or, a VL shown as SEQ ID NO: 8 or a variant thereof;
(e) a VH shown as SEQ ID NO: 9 or a variant thereof, and/or, a VL shown as SEQ ID NO: 10 or a variant thereof;
wherein the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (for example replacement, deletion or addition of 1, 2, 3, 4 or 5 amino acids) as compared to the sequence from which the variants are derived; and preferably, the replacement is conservative replacement.

In some embodiments, the antibody or antigen-binding fragment thereof further comprises:
(a) a human immunoglobulin heavy chain constant region (CH) or a variant thereof, wherein the variant has replacement, deletion or addition of one or more amino acids (for example, replacement, deletion or addition of up to 20, up to 15, up to 10 or up to 5 amino acids; for example, replacement, deletion or addition of 1, 2, 3, 4 or 5 amino acids) as compared to a wild-type sequence from which the variant is derived; and
(b) a human immunoglobulin light chain constant (CL) or a variant, wherein the variant has replacement, deletion or addition of one or more amino acids (for example replacement, deletion or addition of up to 20, up to 15, up to 10 or up to 5 amino acids; for example, replacement, deletion or addition of 1, 2, 3, 4 or 5 amino acids) as compared to the wild-type sequence from which the variant is derived.

In some embodiments, the heavy chain constant region is an IgG heavy chain constant region, for example an IgG1, IgG2, IgG3 or IgG4 heavy chain constant region, for example a human IgG1 heavy chain constant region or a human IgG4 heavy chain constant region. In some embodiments, the antibody or antigen-binding fragment thereof comprises a heavy chain constant region (CH) shown as SEQ ID NO: 43 or a variant thereof, and the variant has conservative replacement of up to 20 amino acids (for example, conservative replacement of up to 15, up to 10 or up to 5 amino acids; for example, conservative replacement of 1, 2, 3, 4 or 5 amino acids) as compared to SEQ ID NO: 43.

In some embodiments, the light chain constant region is a k light chain constant region. In some embodiments, the antibody or antigen-binding region thereof comprises a light chain constant region (CL) shown as SEQ ID NO: 44 or a variant thereof, and the variant has conservative replacement of up to 20 amino acids (for example, conservative replacement of up to 15, up to 10 or up to 5 amino acids; for example, conservative replacement of 1, 2, 3, 4 or 5 amino acids) as compared to SEQ ID NO: 44.

In some embodiments, the antibody or antigen-binding fragment thereof comprises a heavy chain constant region (CH) shown as SEQ ID NO: 43 or 45, and a light chain constant region (CL) shown as SEQ ID NO: 44.

In some embodiments, the antibody or antigen-binding fragment thereof comprises:
(1) a heavy chain comprising VH sequence shown as SEQ ID NO: 1 and heavy chain constant region (CH) shown as SEQ ID NO: 43, and, a light chain comprising VL sequence shown as SEQ ID NO: 2 and light chain constant region (CL) shown as SEQ ID NO: 44;
(2) a heavy chain comprising VH sequence shown as SEQ ID NO: 3 and heavy chain constant region (CH) shown as SEQ ID NO: 43 or 45, and, a light chain comprising VL sequence shown as SEQ ID NO: 4 and light chain constant region (CL) shown as SEQ ID NO: 44;
(3) a heavy chain comprising VH sequence shown as SEQ ID NO: 5 and heavy chain constant region (CH) shown as SEQ ID NO: 43, and, a light chain comprising VL sequence shown as SEQ ID NO: 6 and light chain constant region (CL) shown as SEQ ID NO: 44;
(4) a heavy chain comprising VH sequence shown as SEQ ID NO: 7 and heavy chain constant region (CH) shown as SEQ ID NO: 43, and, a light chain comprising VL sequence shown as SEQ ID NO: 8 and light chain constant region (CL) shown as SEQ ID NO: 44; or
(5) a heavy chain comprising VH sequence shown as SEQ ID NO: 9 and heavy chain constant region (CH) shown as SEQ ID NO: 43, and, a light chain comprising VL sequence shown as SEQ ID NO: 10 and light chain constant region (CL) shown as SEQ ID NO: 44.

In the antibody-drug conjugate, the cytotoxic drug may be linked to the antibody or antigen-binding fragment through the linker (like “M-L-E” fragment shown in the present application).

In some embodiments, M is

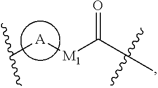

wherein ring A is a 5-6 membered aliphatic heterocyclic ring, or 5-20 membered aromatic ring system, and the aliphatic heterocyclic ring and aromatic ring systems are optionally substituted by one or more members selected from the group consisting of oxo (═O), halogen, cyano, amino, carboxyl, mercapto and C₁₋₆ alkyl; and M₁ is selected from a single bond, C₁₋₂₀ alkylene, C₂₋₂₀ alkenylene and C₂₋₂₀ alkynylene.

In some embodiments, M is

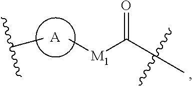

wherein ring A is a 5-membered aliphatic heterocyclic ring, a 6-membered aromatic heterocyclic ring, or a polycyclic ring formed by linking more than one 6-membered aromatic heterocyclic ring and a benzene ring through a single bond, and the aliphatic heterocyclic ring is optionally substituted by one or more members selected from the group consisting of oxo (═O), halogen and C₁₋₄ alkyl; and M₁ is selected from a single bond, C₃₋₁₀ alkylene, C₃₋₁₀ alkenylene and C₃₋₁₀ alkynylene.

In some embodiments, M is

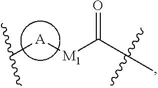

wherein ring A is selected from

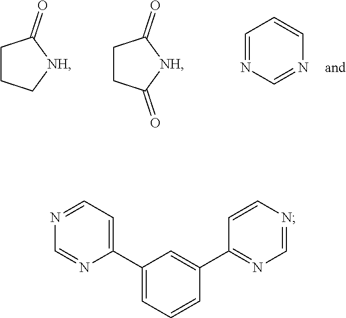 [SMILES: O=C1CCCN1 ; O=C1CCC(=O)N1 ; c1cncnc1 ; c1cc(-c2ccncn2)cc(-c2ccncn2)c1]

and M₁ is selected from a single bond, C₅₋₈ alkylene, C₅₋₈ alkenylene and C₅₋₈ alkynylene.

In some embodiments, M is selected from the following structures:

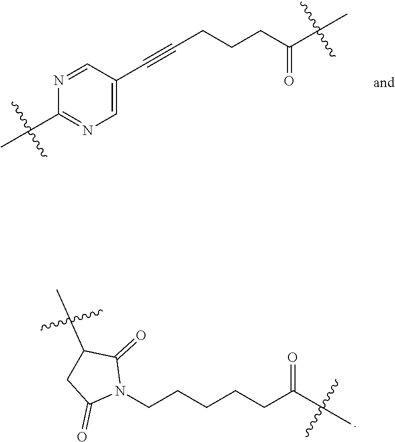 [SMILES: *C(=O)CCCC#Cc1cnc(*)nc1 ; *C(=O)CCCCCN1C(=O)CC(*)C1=O]

In some embodiments, M is selected from the following structures:

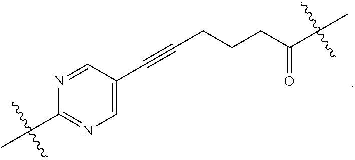 [SMILES: *C(=O)CCCC#Cc1cnc(*)nc1]

In some embodiments, L is selected from a structure comprising one or more of C₁₋₆ alkylene, —N(R′)—, carbonyl, —O—, Val, Cit, Phe, Lys, Lys(COCH₂CH₂(OCH₂CH₂)ₛOCH₃), D-Val, Leu, Gly, Ala, Asn, Val-Cit, Val-Ala, Val-Lys, Val-Lys(Ac), Phe-Lys, Phe-Lys(Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly (SEQ ID NO: 48), Gly-Phe-Leu-Gly (SEQ ID NO: 49), Gly-Gly-Val-Ala (SEQ ID NO: 50), Gly-Gly-Gly-Gly-Gly (SEQ ID NO: 51),

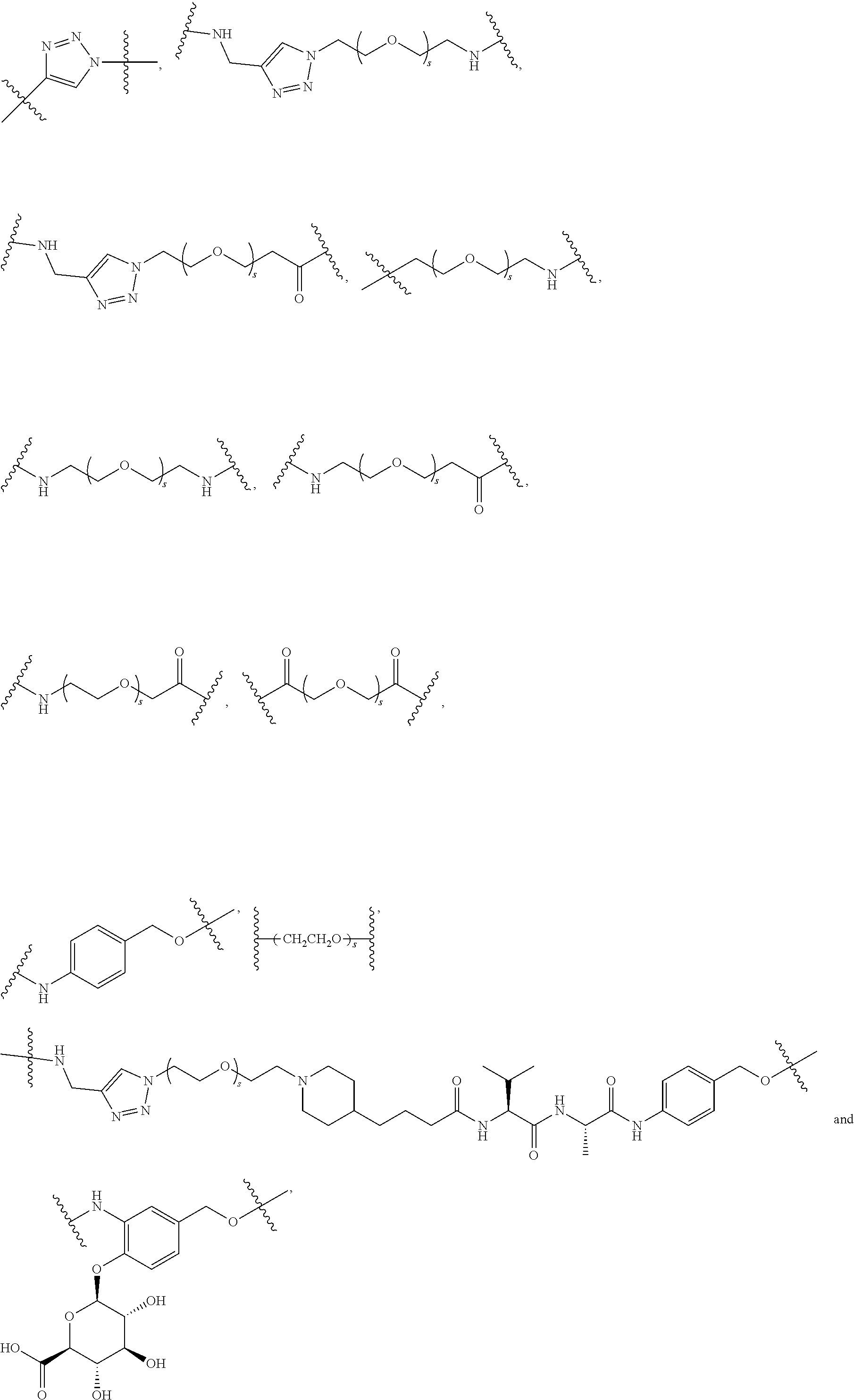

wherein R′ represents hydrogen, C₁₋₆ alkyl or alkyl containing —(CH₂CH₂O)r-; r is selected from an integer from 1-10; and s is selected from an integer from 1-20.

In some embodiments, L is selected from a structure comprising one or more of C₁₋₆ alkylene, —N(R′)—, carbonyl, —O—, Val, Cit, Phe, Lys, Lys(COCH₂CH₂(OCH₂CH₂)ₛOCH₃), D-Val, Leu, Gly, Ala, Asn, Val-Cit, Val-Ala, Val-Lys, Val-Lys(Ac), Phe-Lys, Phe-Lys(Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly (SEQ ID NO: 48), Gly-Phe-Leu-Gly (SEQ ID NO: 49), Gly-Gly-Val-Ala (SEQ ID NO: 50), Gly-Gly-Gly-Gly-Gly (SEQ ID NO: 51),

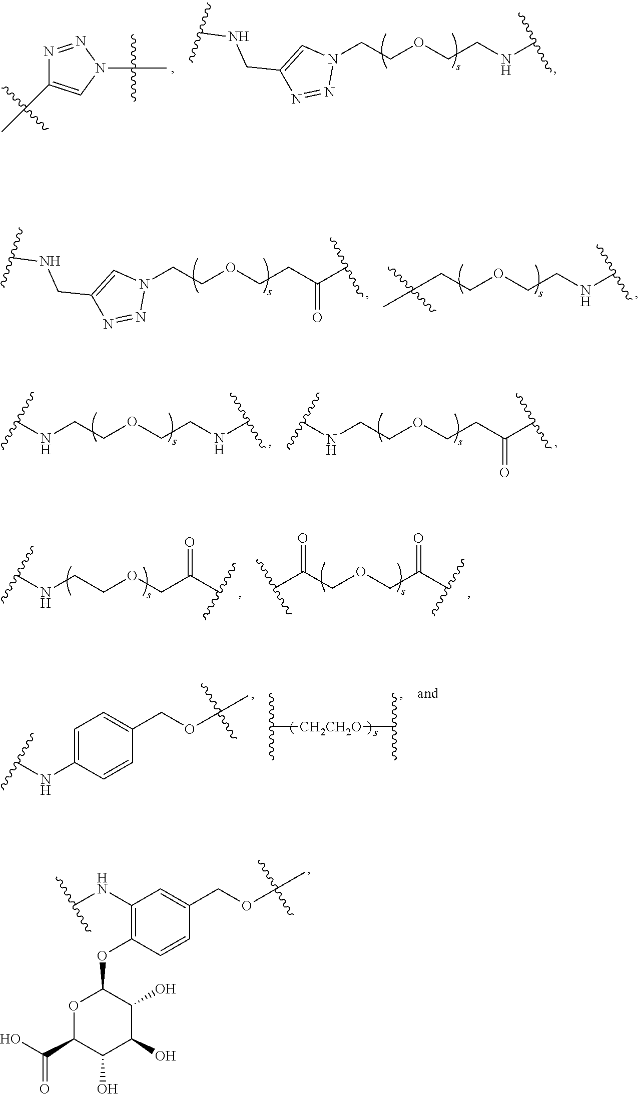

wherein R′ represents hydrogen, C₁₋₆ alkyl or alkyl containing —(CH₂CH₂O)r-; r is selected from an integer from 1-10; and s is selected from an integer from 1-20.

In some embodiments, L is selected from a structure comprising one or more of C₁₋₆ alkylene, —NH—, Val, Cit, Phe, Lys, Lys(COCH₂CH₂(OCH₂CH₂)ₛOCH₃), Gly, Val-Cit, Gly-Gly-Phe-Gly (SEQ ID NO: 48),

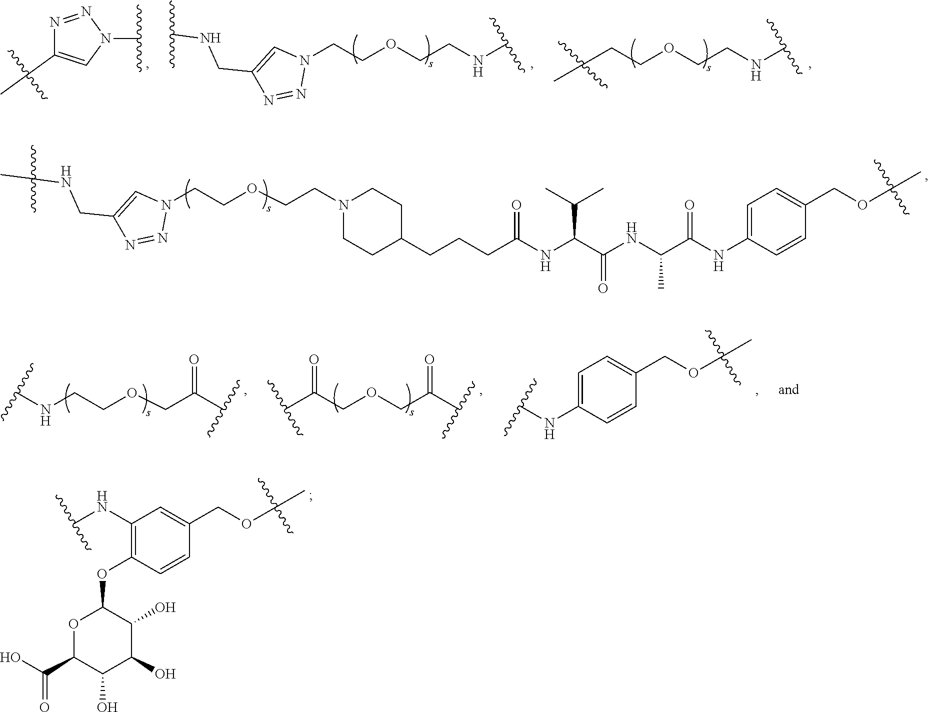

where s is selected from an integer from 1-20.

In some embodiments, L is selected from a structure comprising one or more of C₁₋₆ alkylene, —NH—, Val, Cit, Phe, Lys, Lys(COCH₂CH₂(OCH₂CH₂)ₛOCH₃), Gly, Val-Cit, Gly-Gly-Phe-Gly (SEQ ID NO: 48),

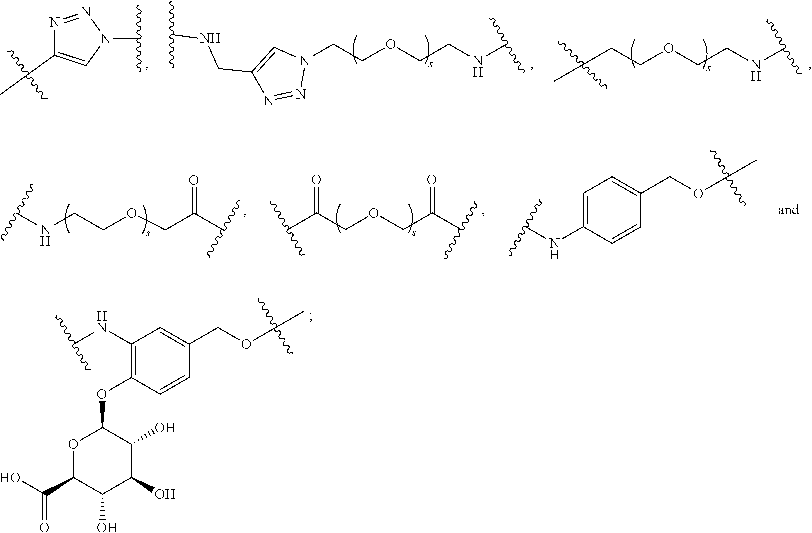

where s is selected from an integer from 1-20.

In some embodiments, L is selected from the following structures:

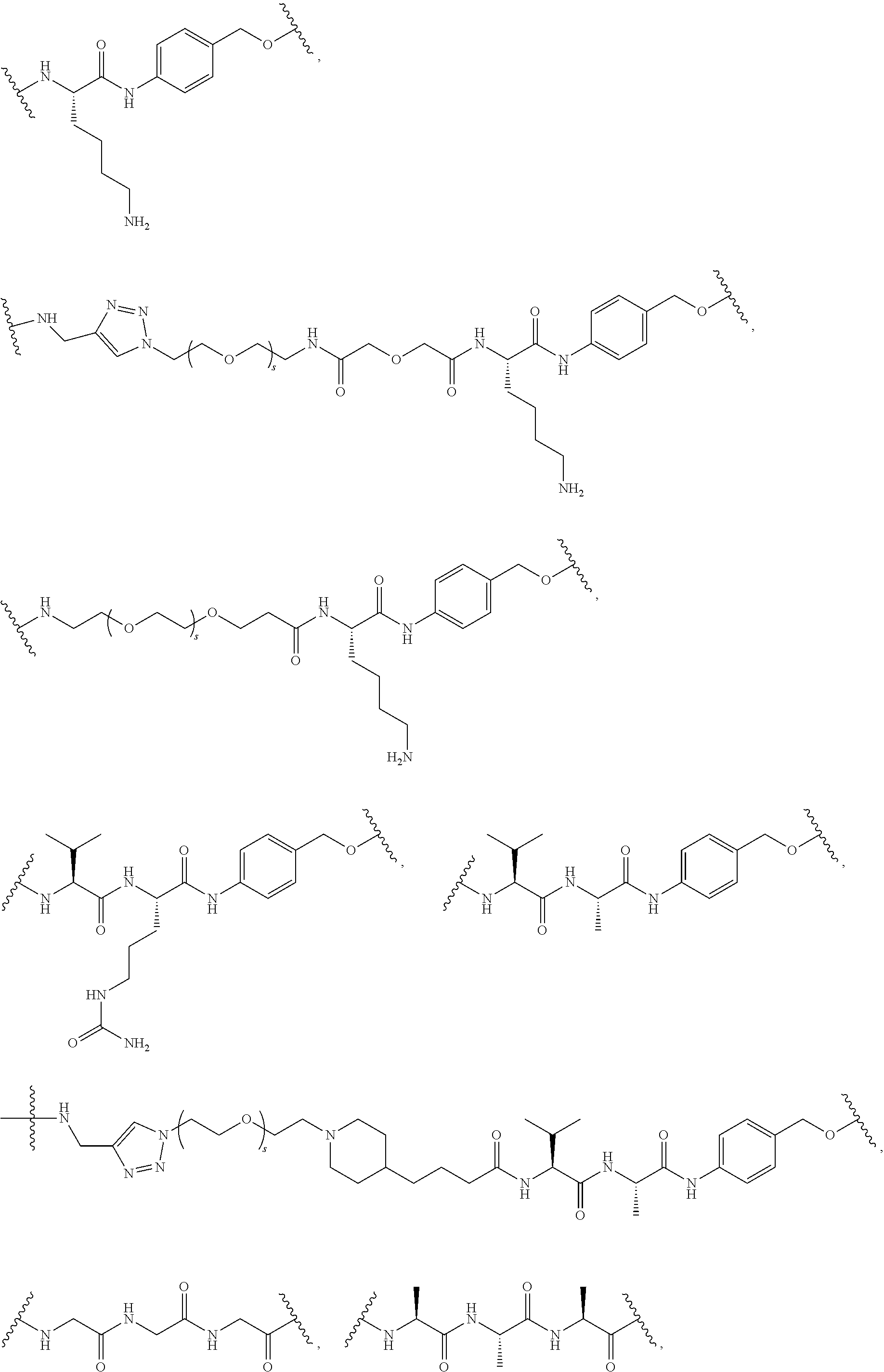

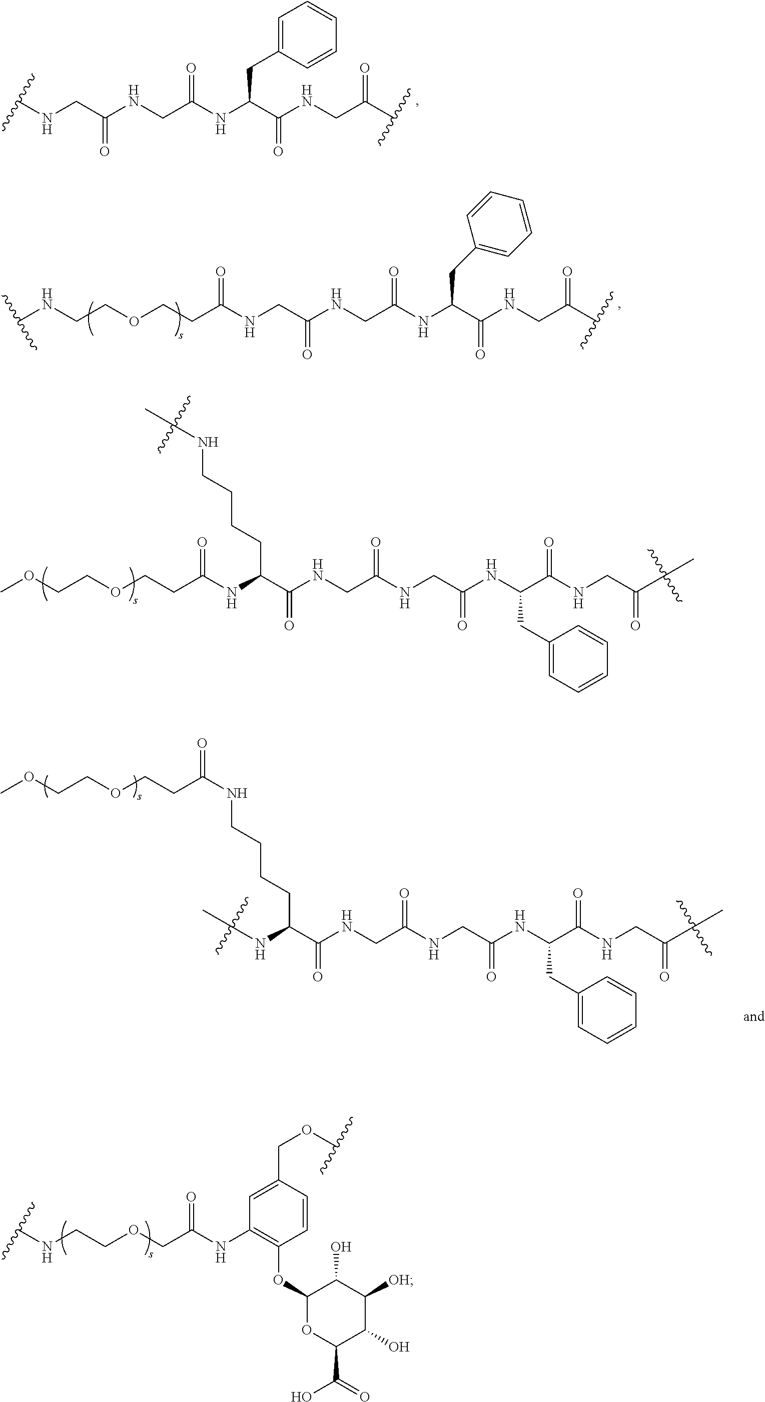

where s is selected from an integer from 1-20.

In some embodiments, L is selected from the following structures:

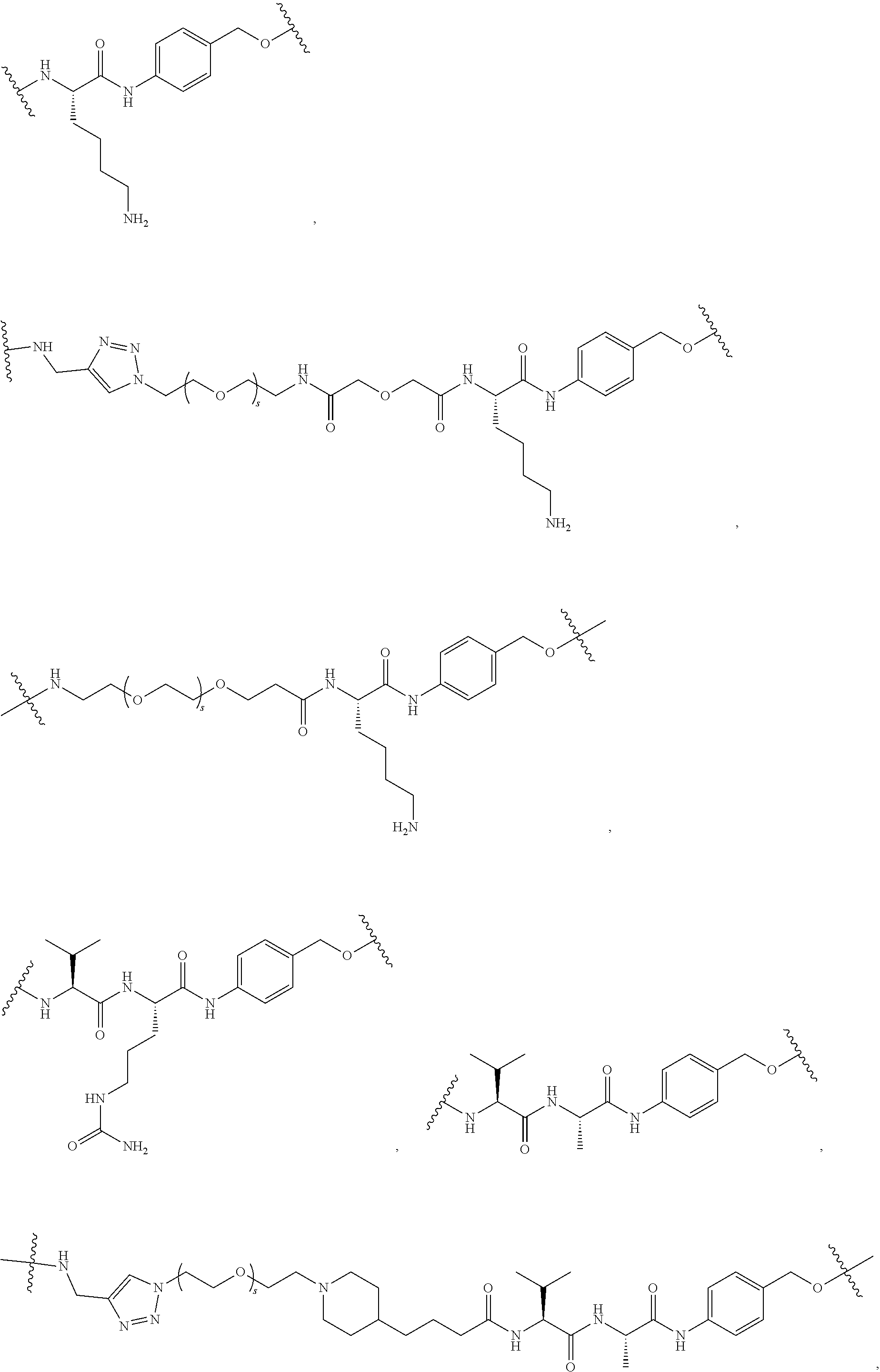

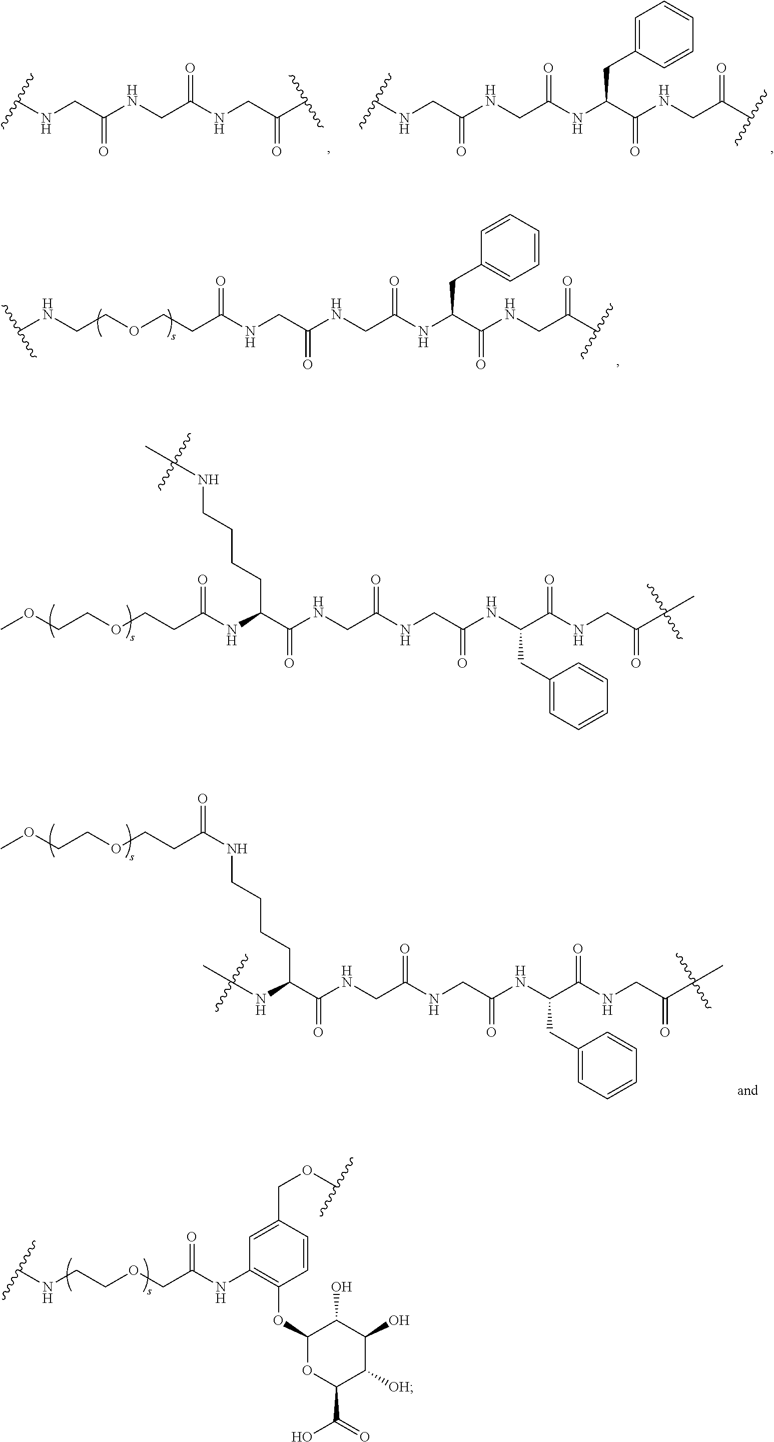

where s is selected from an integer from 1-20.

In some embodiments, L is selected from the following structures:

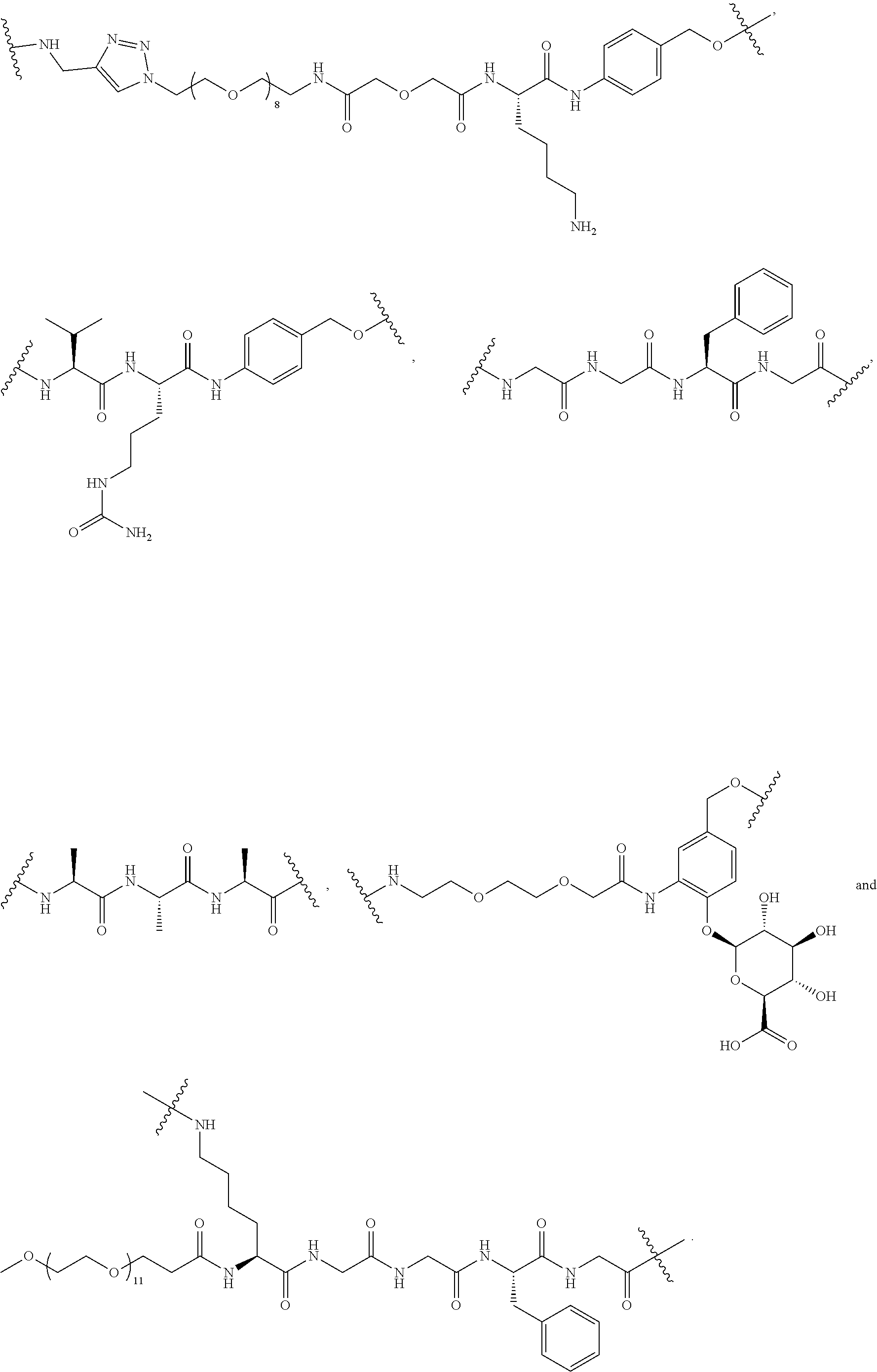

In some embodiments, L is selected from the following structures:

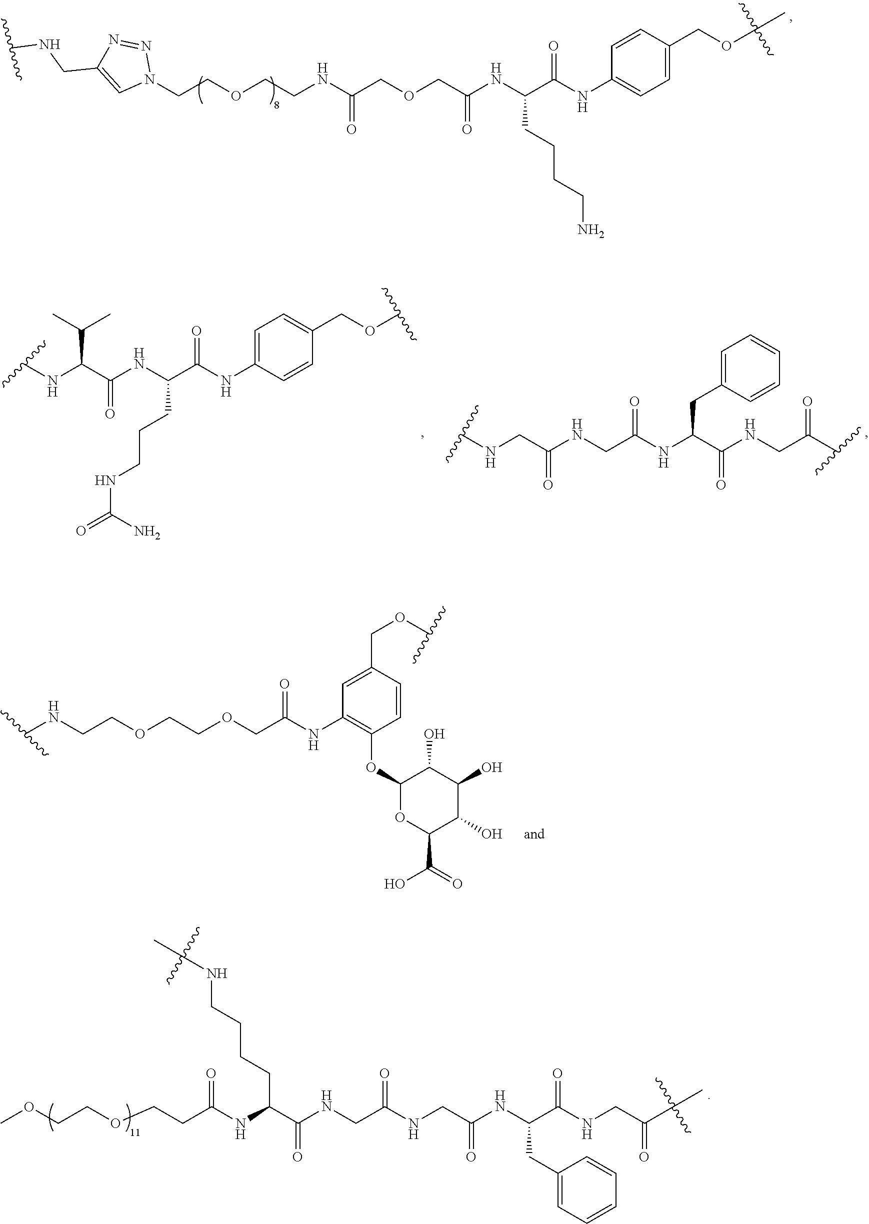

In some embodiments, L is selected from the following structures:

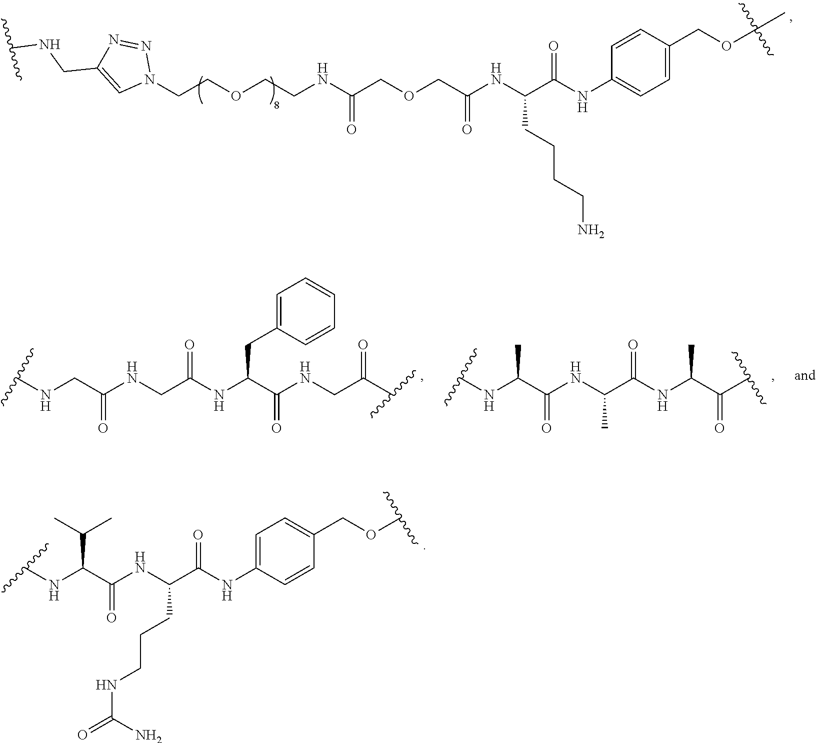

In some embodiments, L is selected from the following structures:

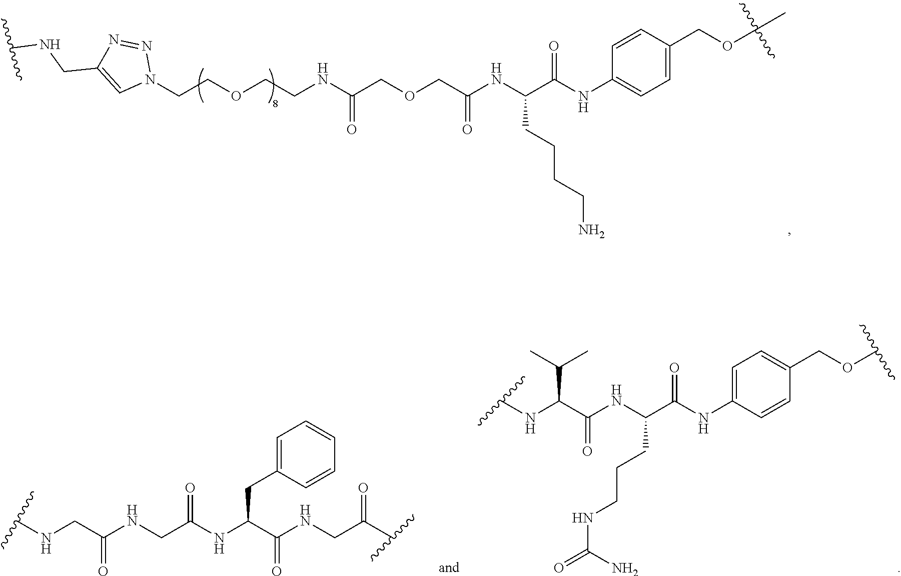

In some embodiments, E is a single bond, —NH—CH₂—, —NH—CH₂—O—CH₂—CO—,

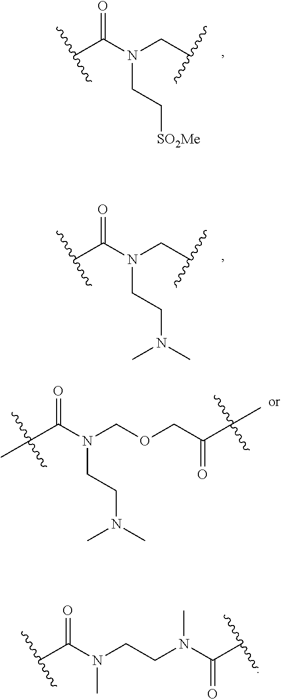

In some embodiments, E is a single bond, —NH—CH₂—, —NH—CH₂—O—CH₂—CO—,

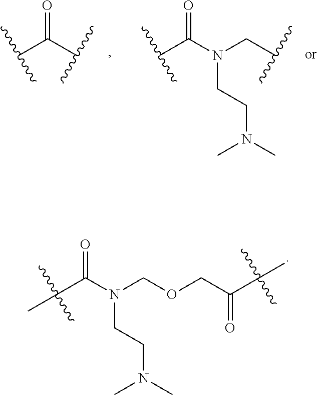

In some embodiments, E is —NH—CH₂—,

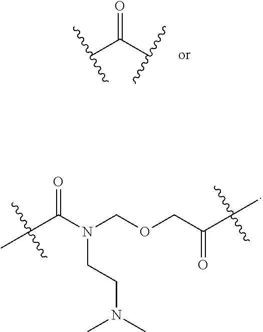

In some embodiments, E is —NH—CH₂— or

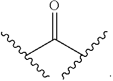 [SMILES: *C(*)=O]

In some embodiments, M is selected from the following structures:

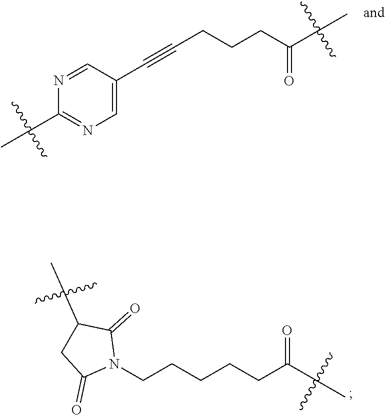 [SMILES: *C(=O)CCCC#Cc1cnc(*)nc1 ; *C(=O)CCCCCN1C(=O)CC(*)C1=O]

L is selected from the following structures:

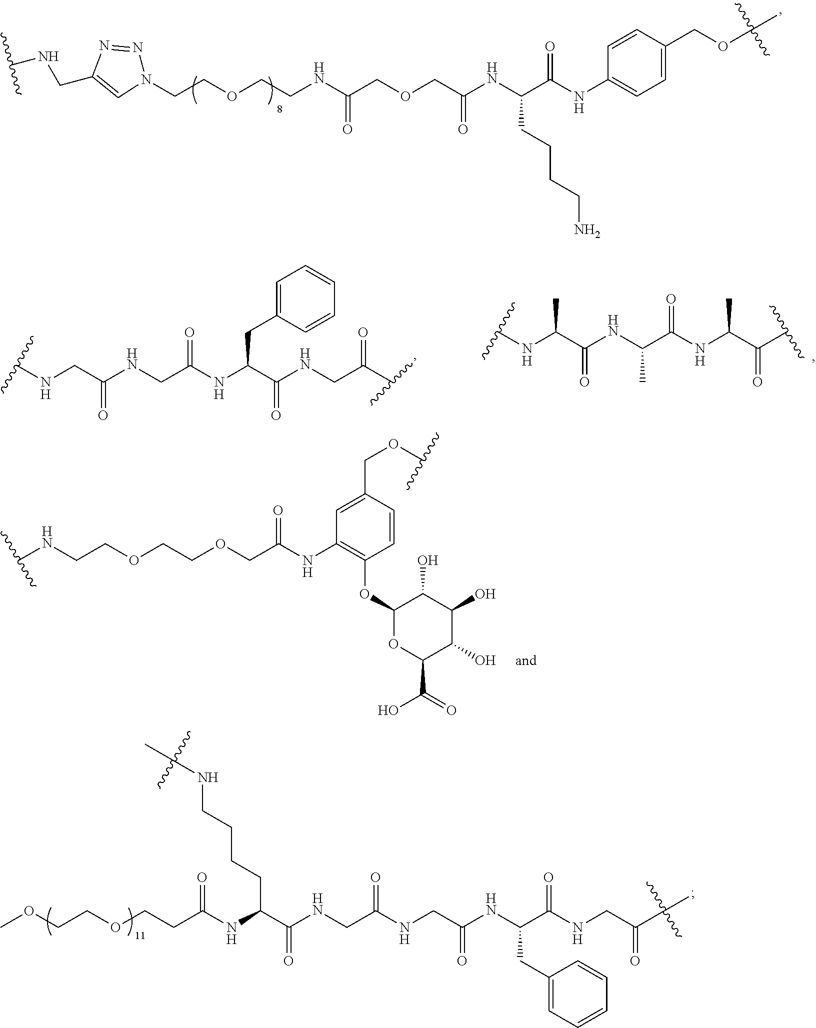

E is —NH—CH₂,

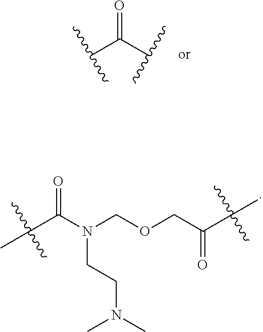

In some embodiments, M is selected from the following structures:

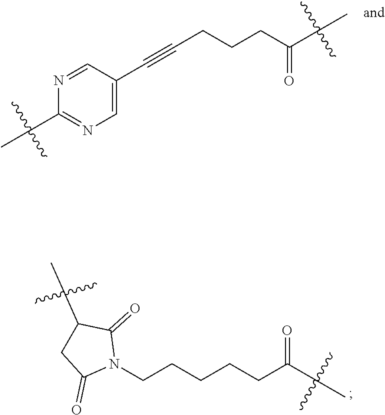 [SMILES: *C(=O)CCCC#Cc1cnc(*)nc1 ; *C(=O)CCCCCN1C(=O)CC(*)C1=O]

L is selected from the following structures:

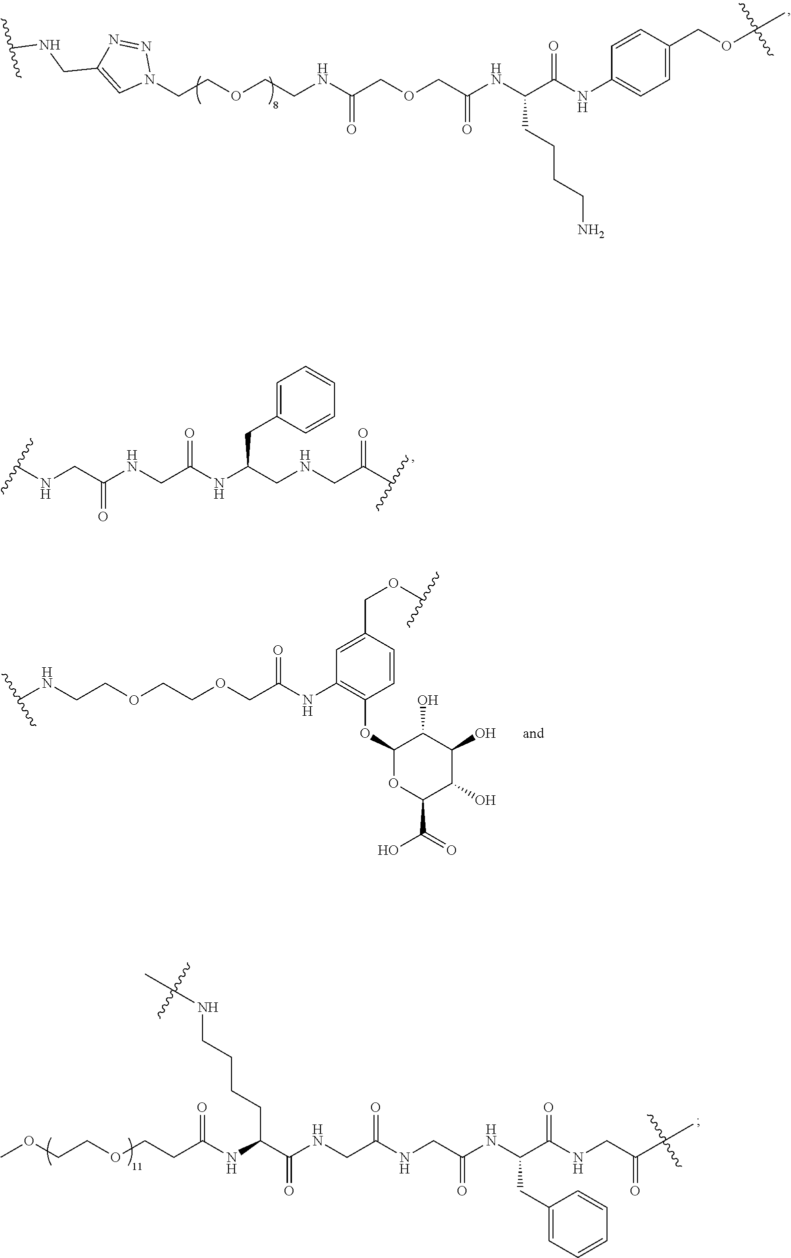

E is —NH—CH₂,

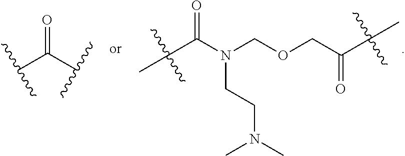

In some embodiments,

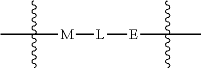

is selected from the following structures:

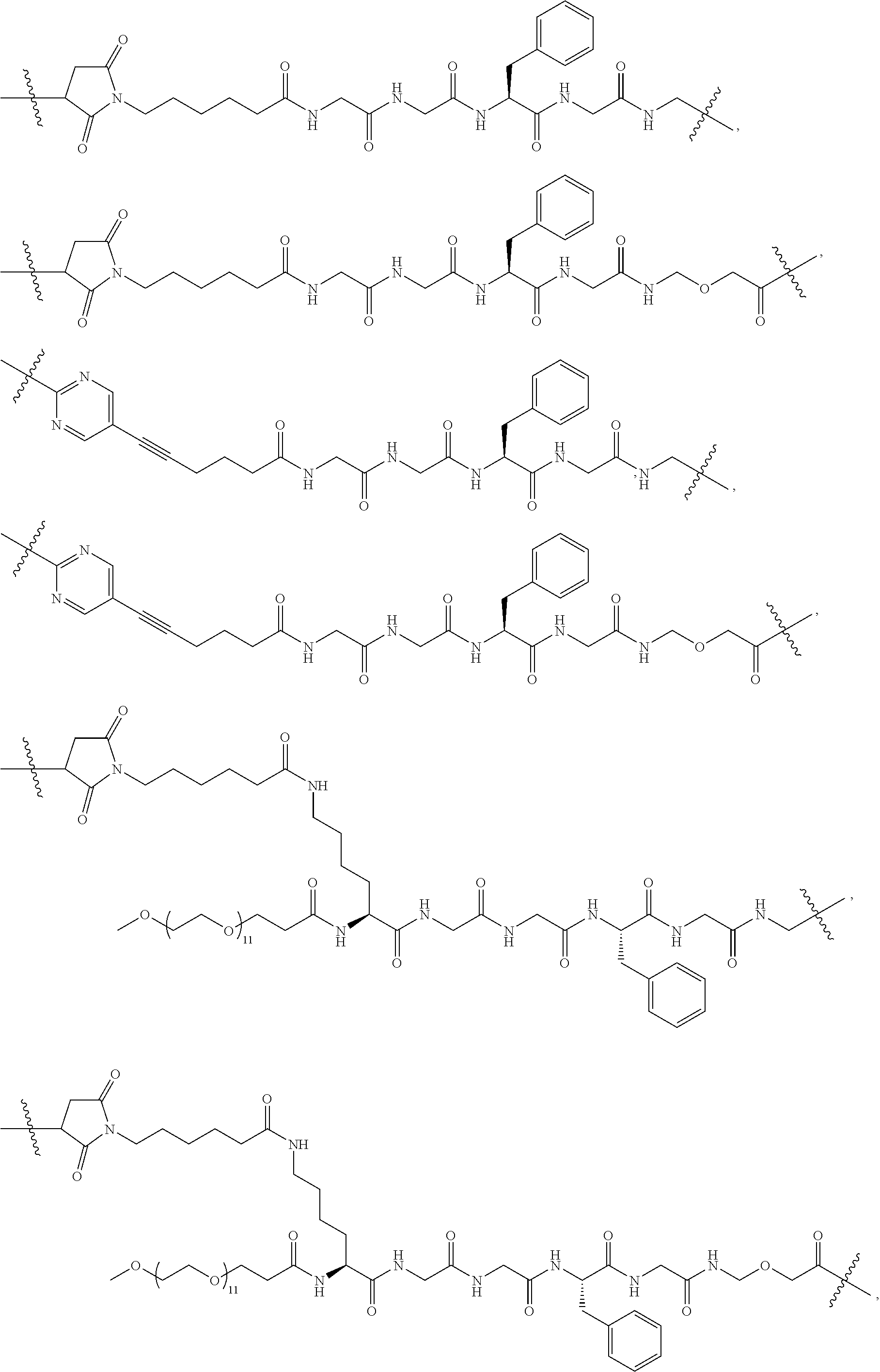

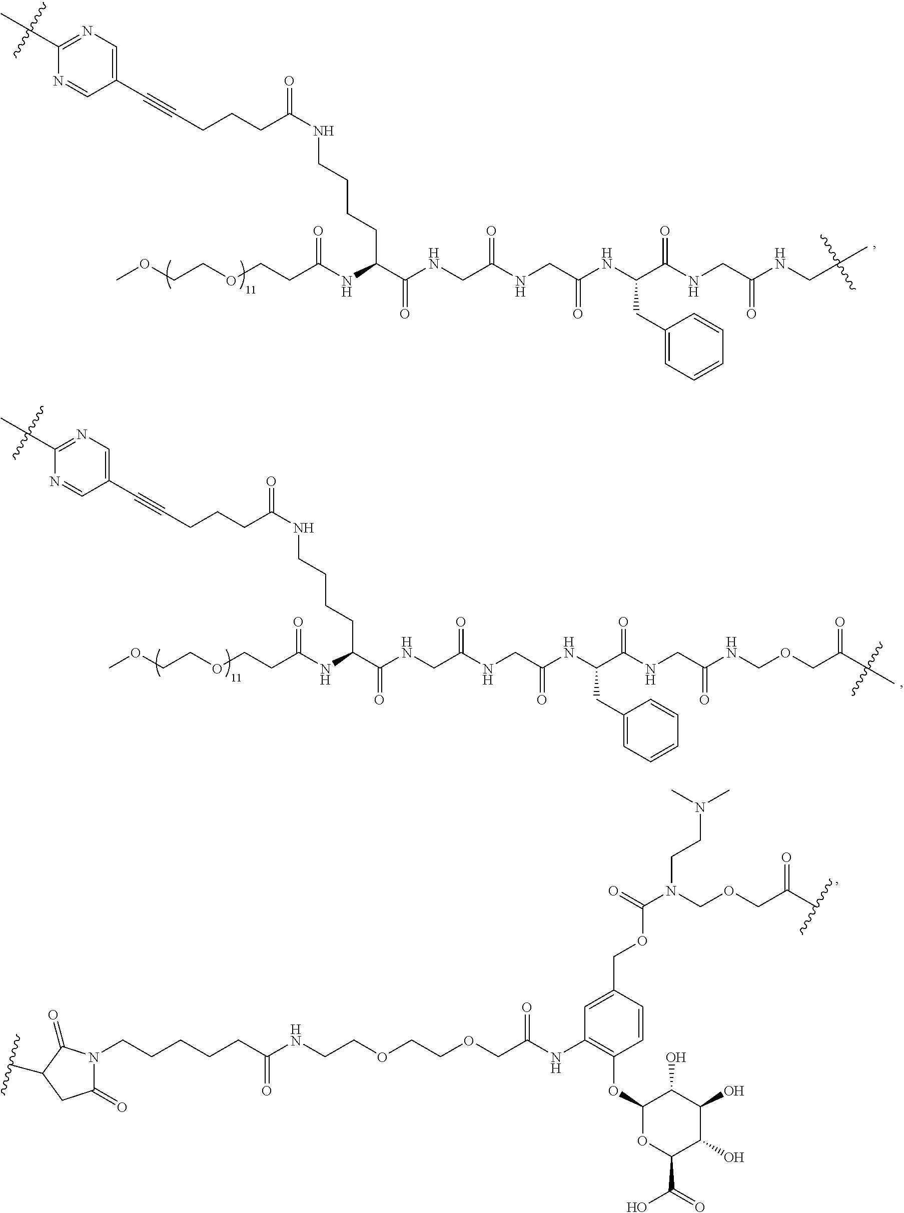

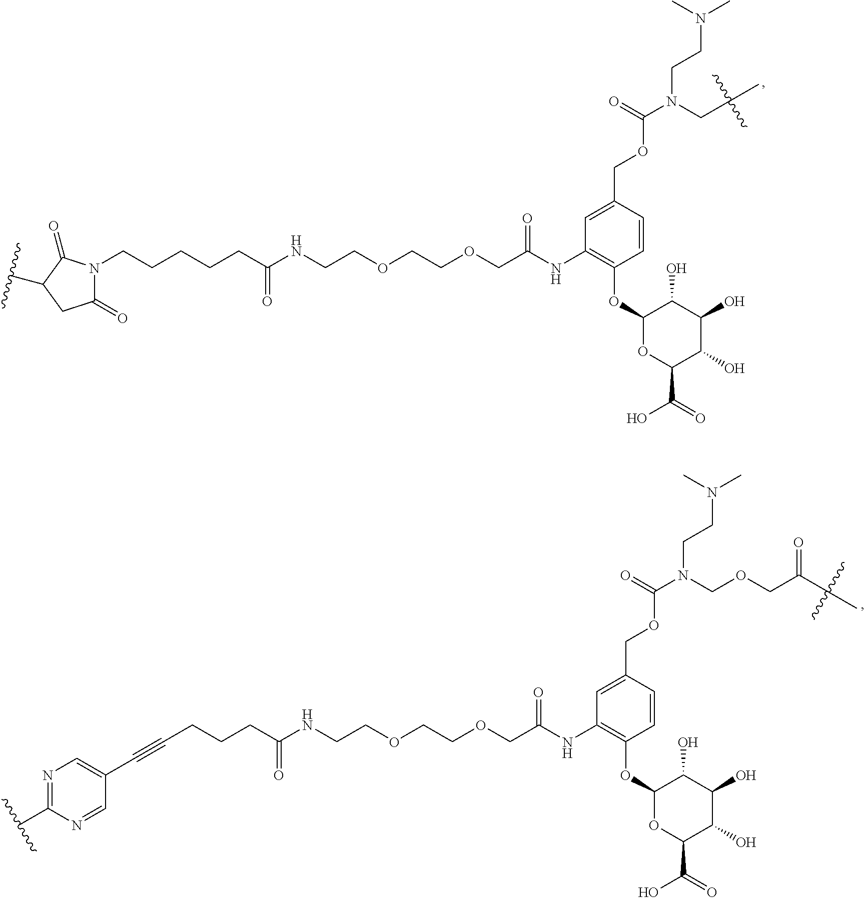

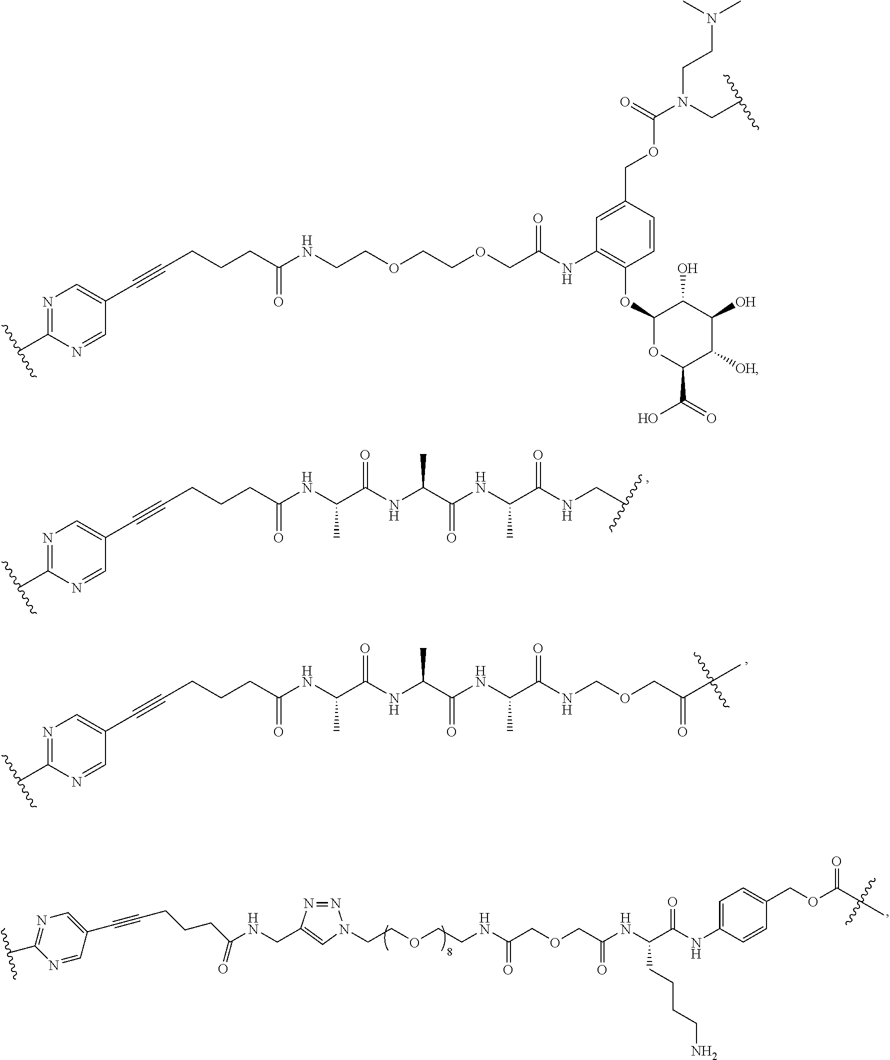

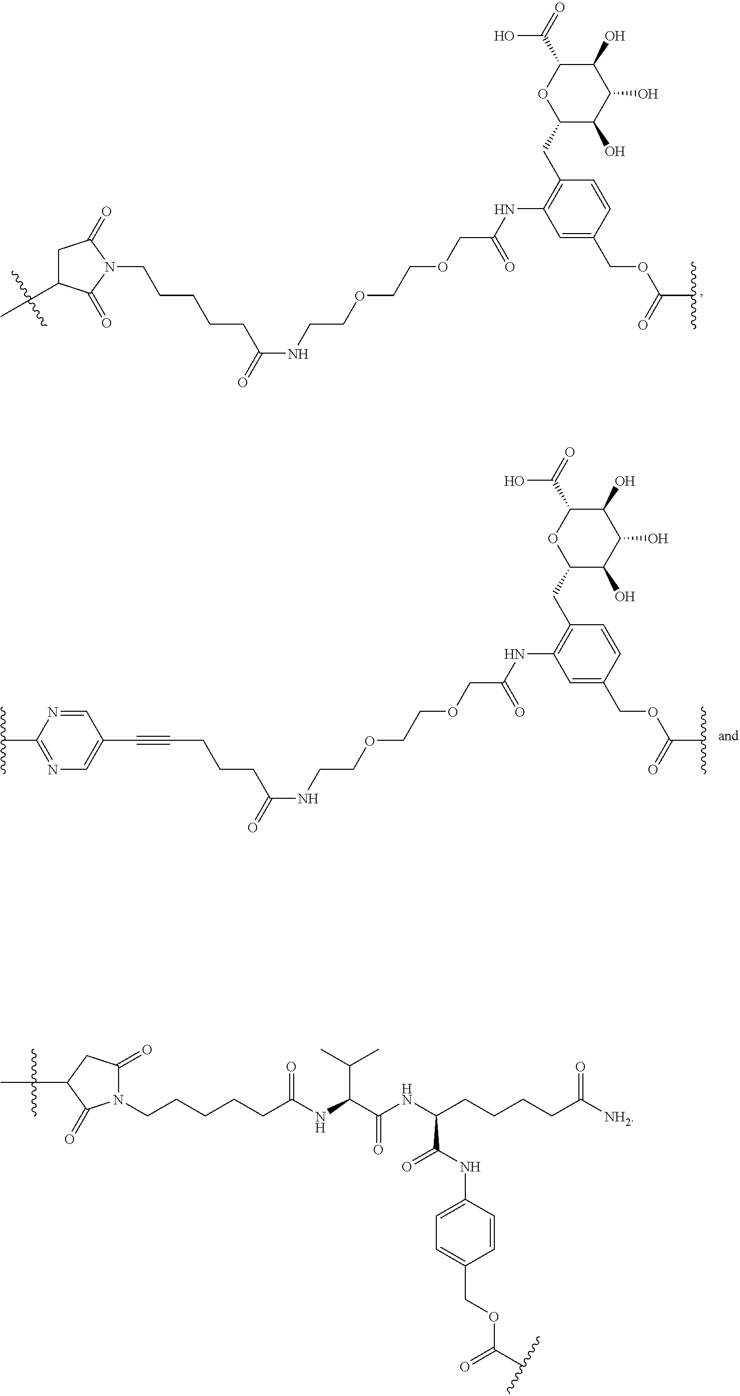

In some embodiments,

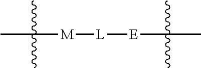

is selected from the following structures:

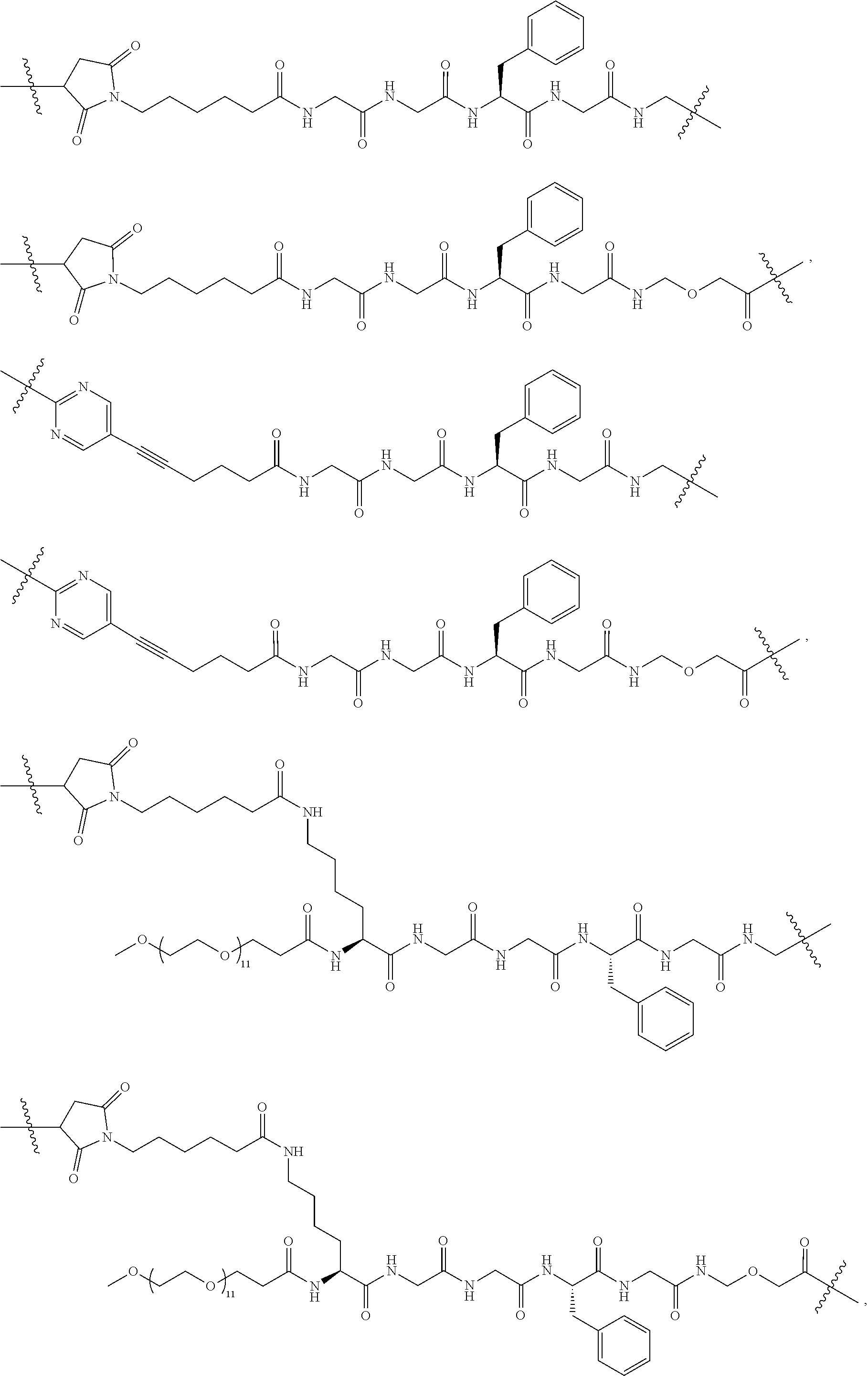

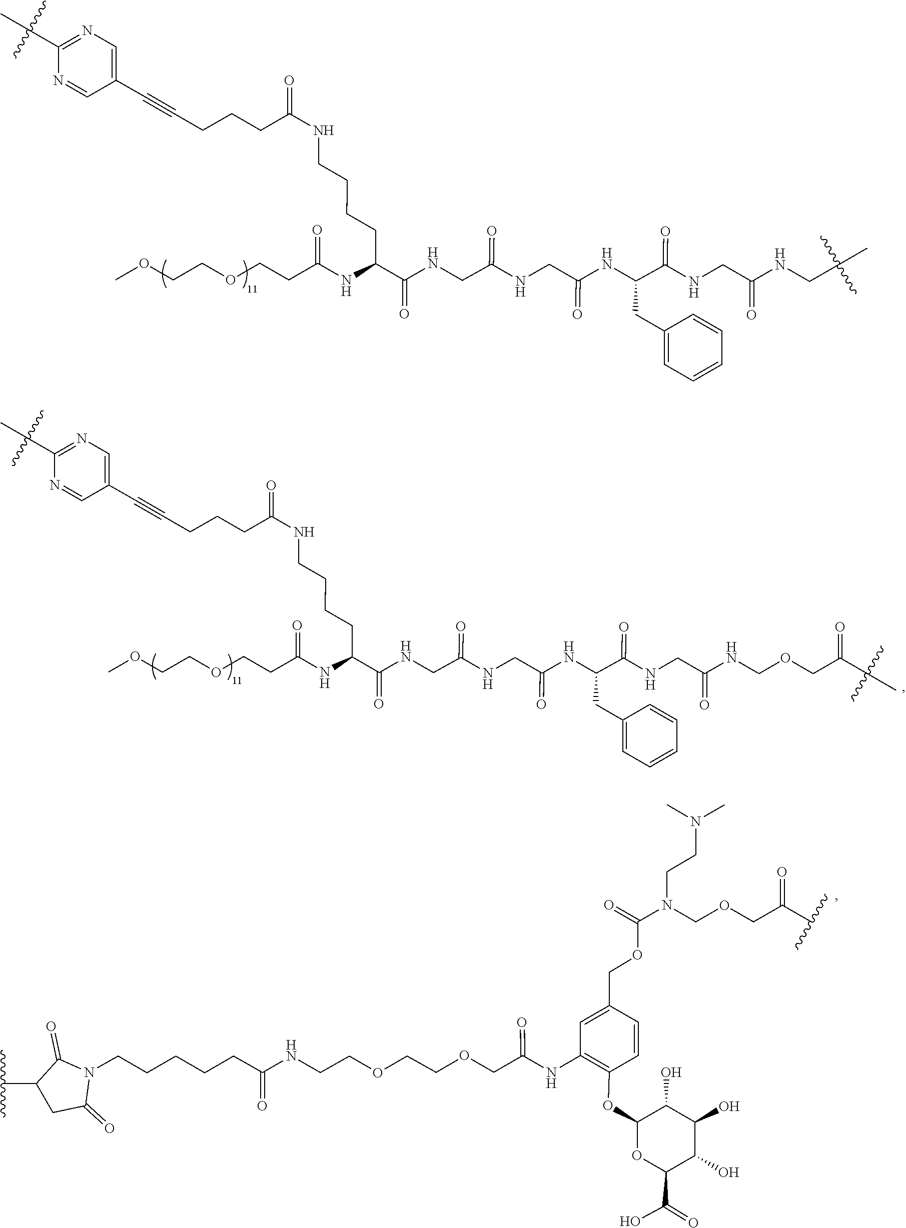

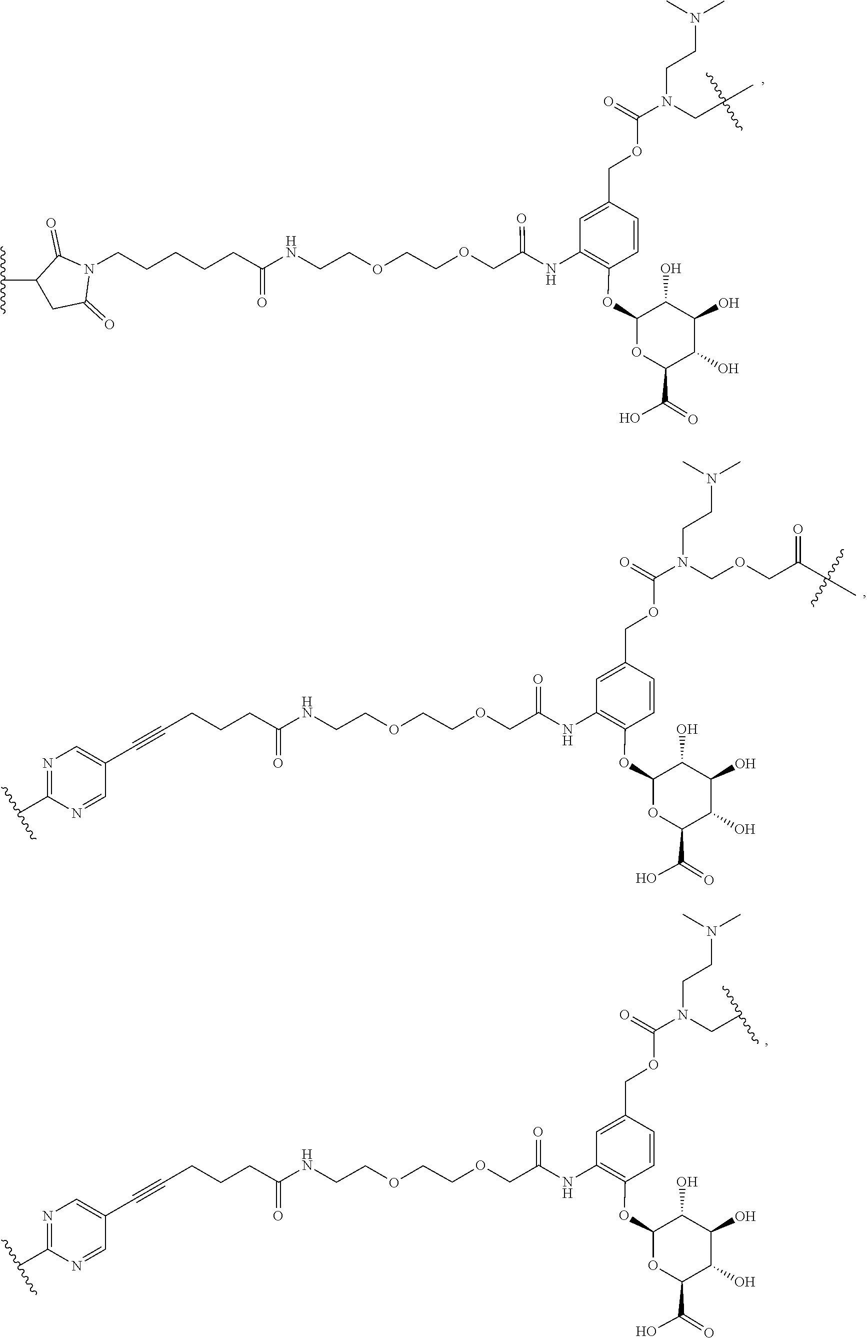

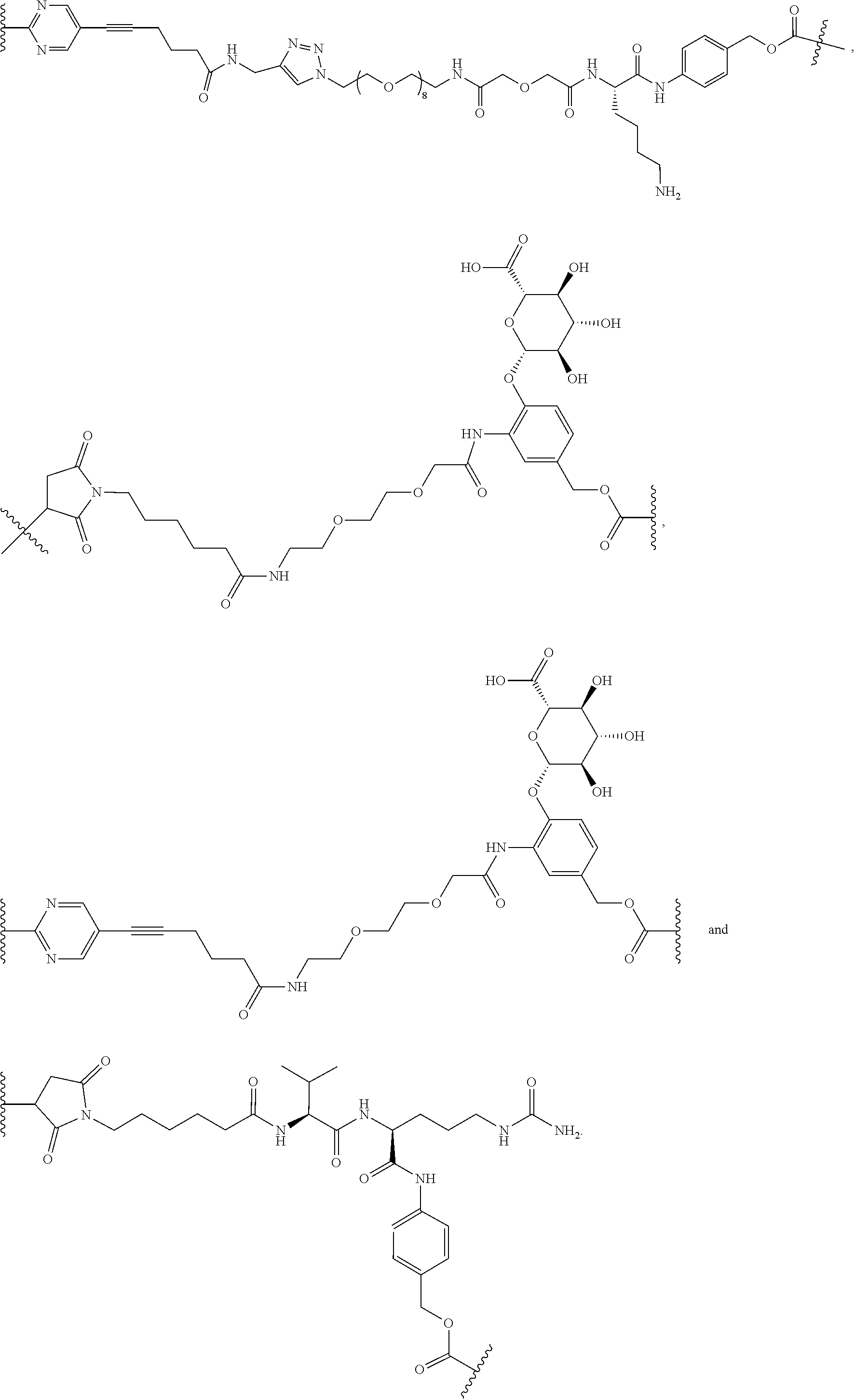

In some embodiments,

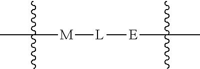

is selected from the following structures:

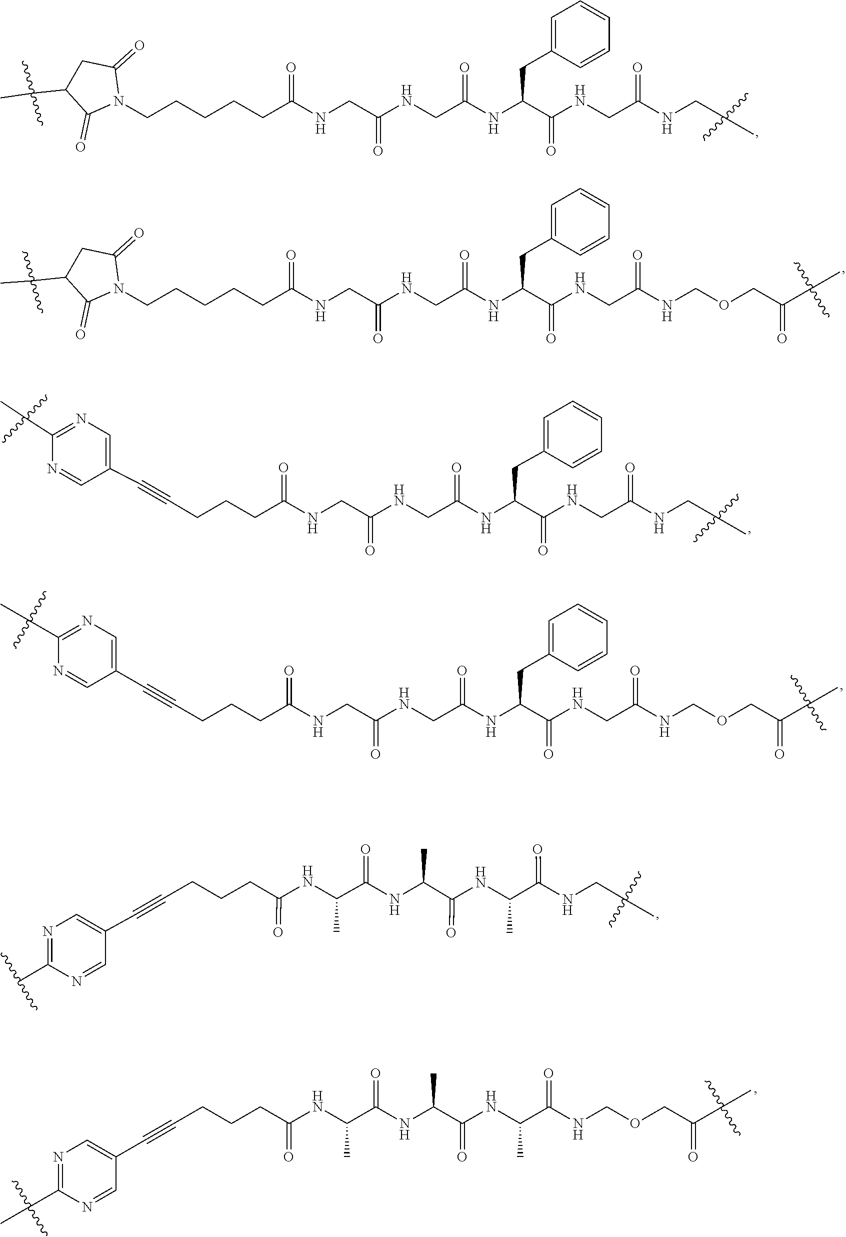 [SMILES: *CNC(=O)CNC(=O)[C@H](Cc1ccccc1)NC(=O)CNC(=O)CNC(=O)CCCCCN1C(=O)CC(*)C1=O ; *C(=O)COCNC(=O)CNC(=O)[C@H](Cc1ccccc1)NC(=O)CNC(=O)CNC(=O)CCCCCN1C(=O)CC(*)C1=O ; *CNC(=O)CNC(=O)[C@H](Cc1ccccc1)NC(=O)CNC(=O)CNC(=O)CCCC#Cc1cnc(*)nc1 ; *C(=O)COCNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)COCNC(=O)[C@H](C)NC(=O)[C@H](C)NC(=O)[C@H](C)NC(=O)CCCC#Cc1cnc(*)nc1]

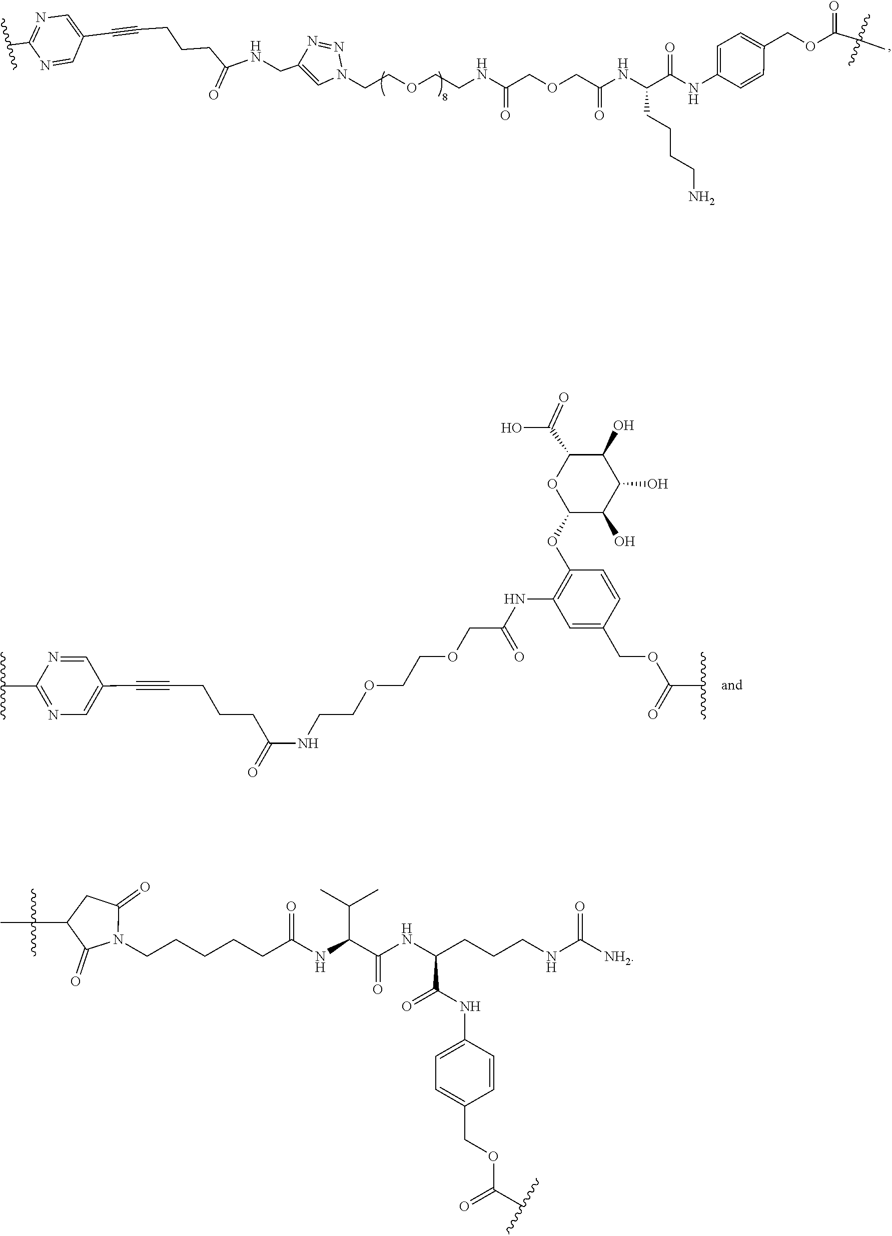

In some embodiments,

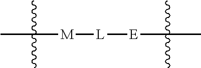

is selected from the following structures:

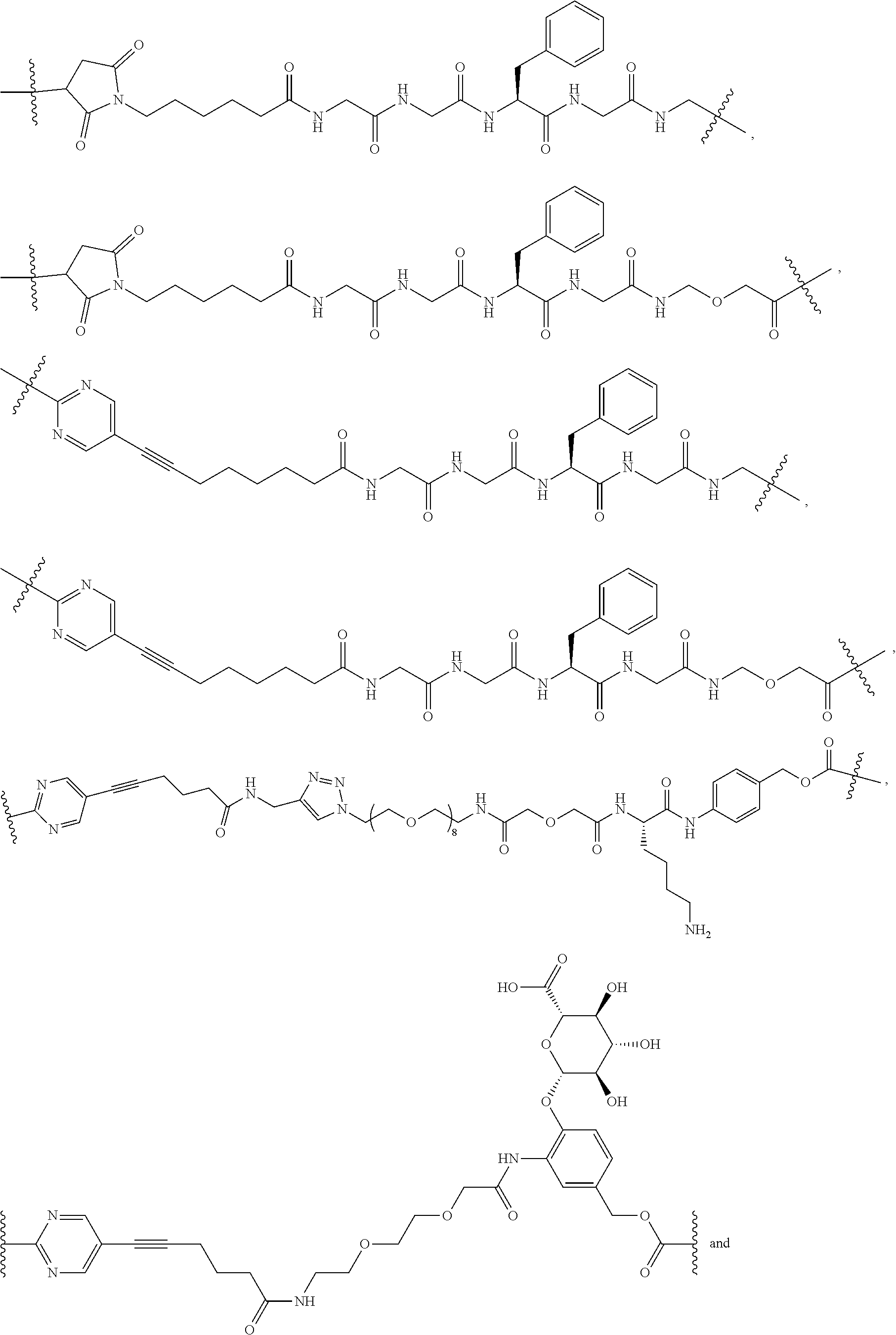

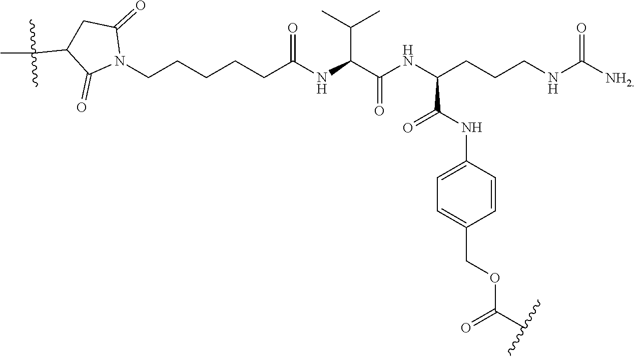 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1]

In some embodiments, the cytotoxic drug is selected from a tubulin inhibitor, a DNA intercalator, a DNA topoisomerase inhibitor and a RNA polymerase inhibitor. In some embodiments, the tubulin inhibitor is an auristatin compound or a maytansinoid compound. In some embodiments, the DNA intercalator is pyrrolobenzodiazepine (PBD). In some embodiments, the DNA topoisomerase inhibitor is a topoisomerase I inhibitor (for example, camptothecin, hydroxycamptothecin, 9-aminocamptothecin, SN-38, irinotecan, topotecan, belotecan, or rubitecan) or a topoisomerase II inhibitor (for example, doxorubicin, PNU-159682, duocarmycin, daunorubicin, mitoxantrone, podophyllotoxin, or etoposide). In some embodiments, the RNA polymerase inhibitor is a-amanitin or a pharmaceutically acceptable salt, ester or analog thereof.

The cytotoxic drug disclosed in the present application usually contains a variety of functional groups, for example hydroxyl (—OH), carboxyl (—COOH), sulfhydryl (—SH), primary amine (—NH₂), secondary amine (—NR_{A}H) or a tertiary amine group (—NR_{B}R_{C}), and R_{A}, R_{B} and R_{C} herein only represent non-hydrogen substitutes on N; and the cytotoxic drug can be linked to the linker in the conjugate through these functional groups.

In some embodiments, the cytotoxic drug is linked to E in the antibody-drug conjugate through —OH, —SH, primary amine group, secondary amine group or tertiary amine group thereon.

In some embodiments, the cytotoxic drug is

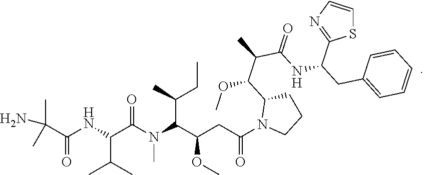

In some embodiments, the cytotoxic drug is selected from the following formulas I and II:

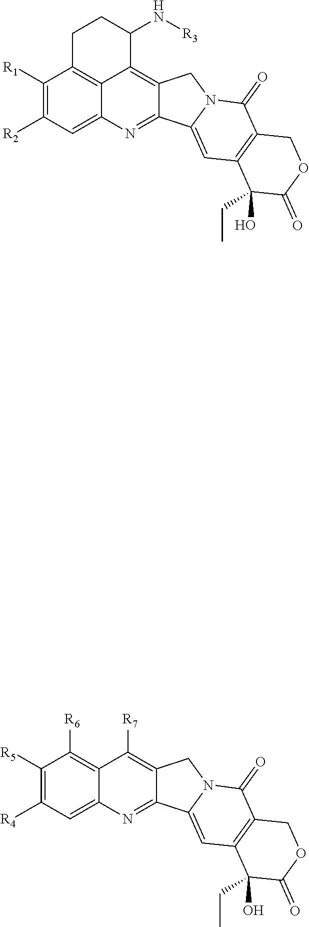

wherein R₁ and R₂ are each independently selected from C₁₋₆ alkyl and halogen;
R₃ is selected from H and —CO—CH₂OH;
R₄ and R₅ are each independently selected from H, halogen and hydroxyl; or R₄ and R₅ are linked to associated carbon atoms to form 5-6 membered oxygen-containing heterocyclic ring;
R₆ is selected from hydrogen and —C₁₋₄ alkylene-NRₐR_{b};
R₇ is selected from hydrogen, C₁₋₆ alkyl and —C₁₋₄ alkylene-NRₐR_{b};
wherein Rₐ and R_{b} are each independently selected from H, C₁₋₆ alkyl, —SO₂—C₁₋₆ alkyl and —CO—C₁₋₆ alkyl at each occurrence.

In some embodiments, the cytotoxic drug is selected from the following formulas I and II:

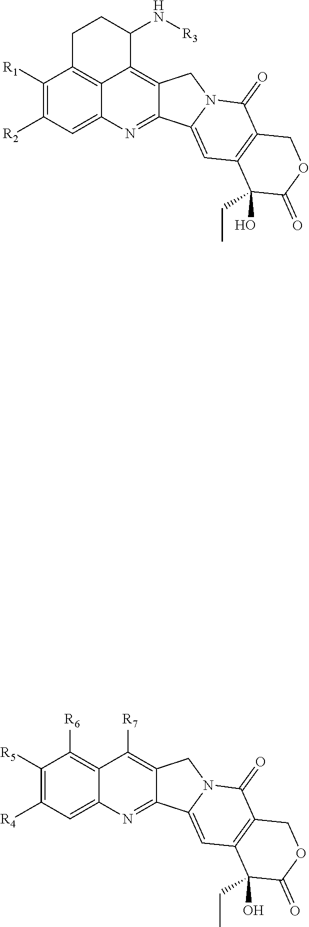

wherein R₁ and R₂ are each independently selected from C₁₋₆ alkyl and halogen;
R₃ is selected from H and —CO—CH₂OH;
R₄ and R₅ are each independently selected from H, halogen and hydroxyl; or R₄ and R₅ are linked to associated carbon atoms to form 5-6 membered oxygen-containing heterocyclic ring;
R₆ is selected from hydrogen and —C₁₋₄ alkylene-NRₐR_{b};
R₇ is selected from C₁₋₆ alkyl and —C₁₋₄ alkylene-NRₐR_{b};
wherein Rₐ and R_{b} are each independently selected from H, C₁₋₆ alkyl, —SO₂—C₁₋₆ alkyl and —CO—C₁₋₆ alkyl at each occurrence.

In some embodiments, the cytotoxic drug is selected from the following compounds:

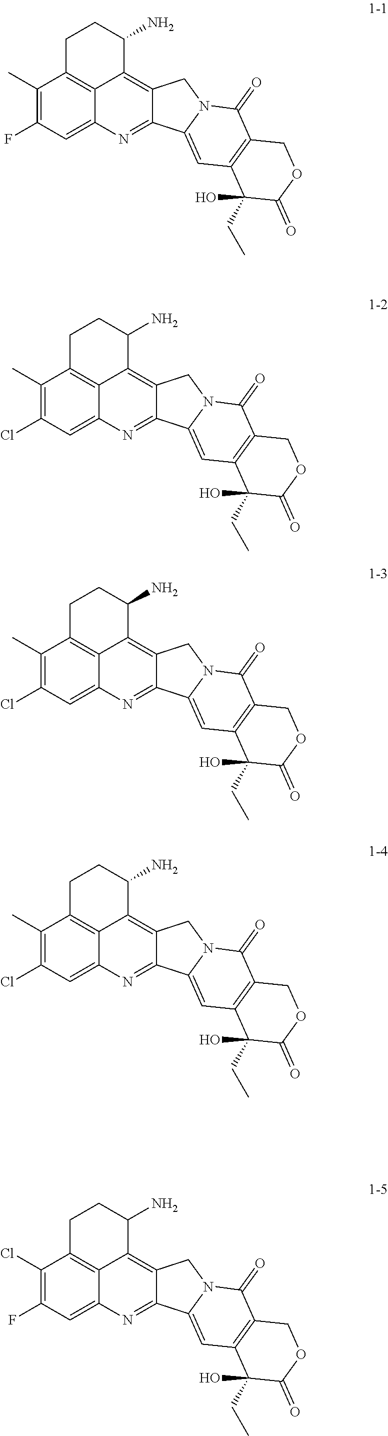 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

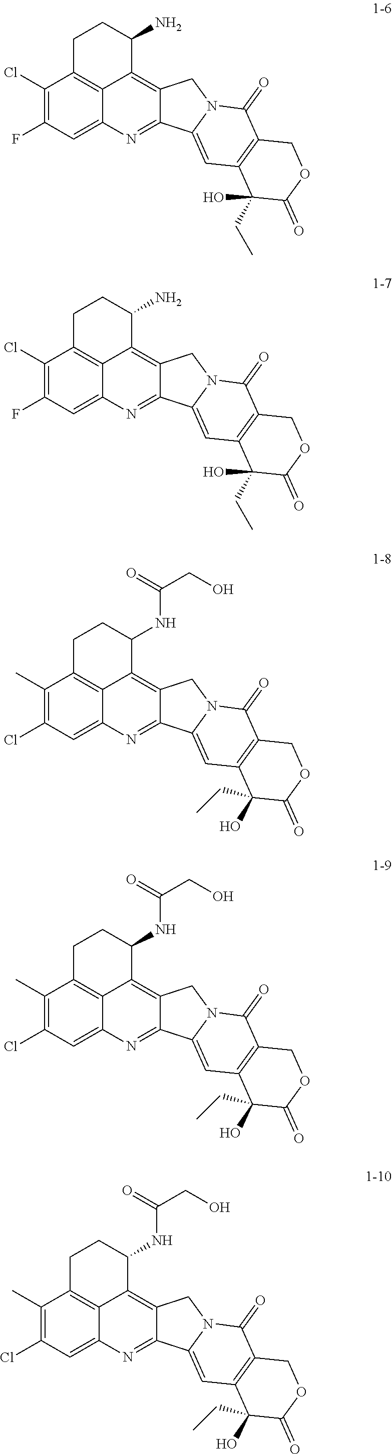 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3]

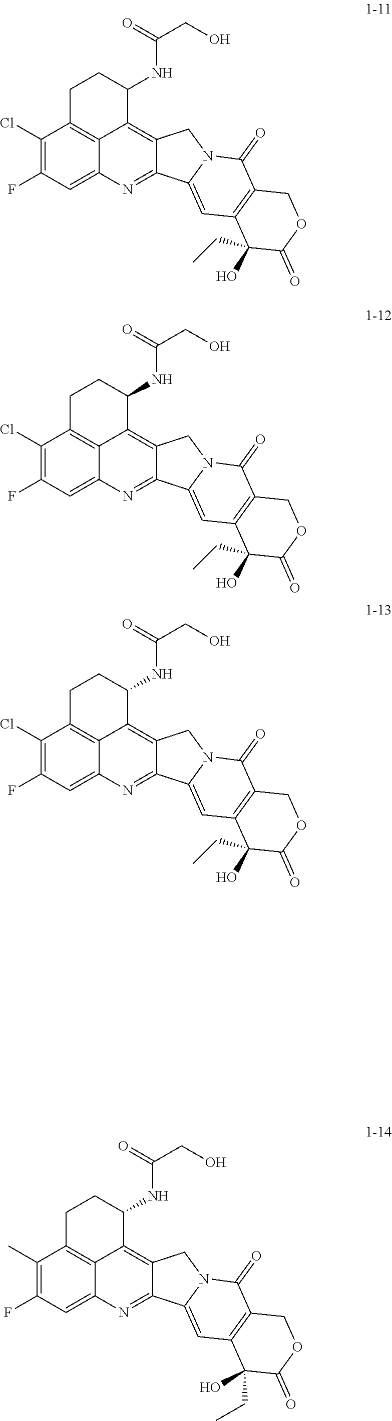 [SMILES: CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3]

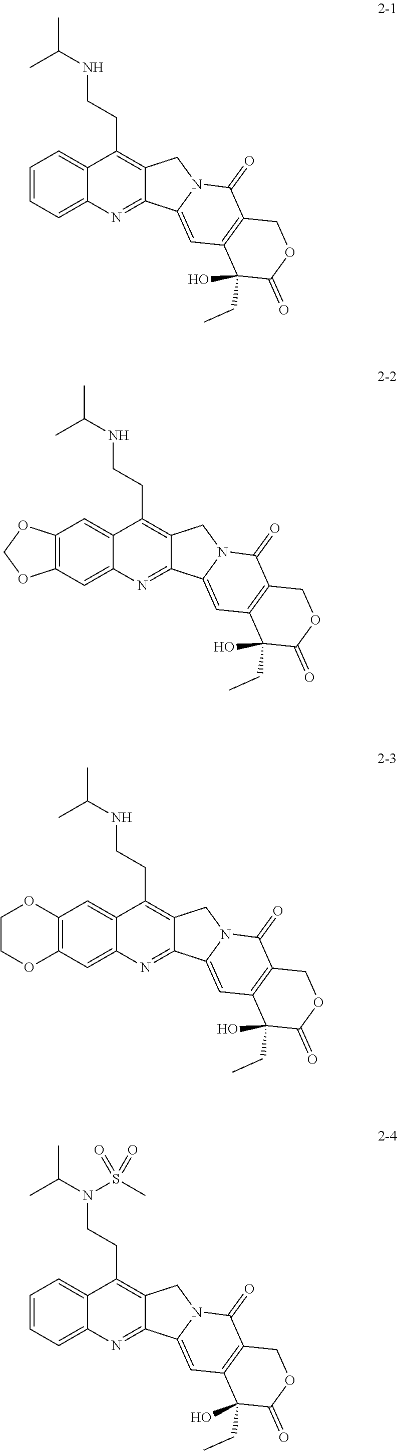 [SMILES: CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3 ; CC[C@@]1(O)C(=O)OCc2c1cc1n(c2=O)Cc2c-1nc1ccccc1c2CCN(C(C)C)S(C)(=O)=O]

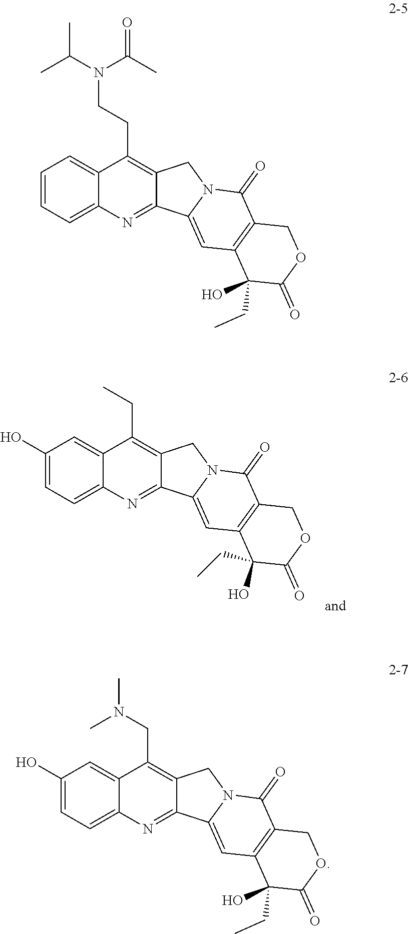 [SMILES: CC[C@@]1(O)C(=O)OCc2c1cc1n(c2=O)Cc2c-1nc1ccccc1c2CCN(C(C)=O)C(C)C ; CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2c-1nc1ccc(O)cc1c2CN(C)C]

In some embodiments, the cytotoxic drug is selected from the following compounds:

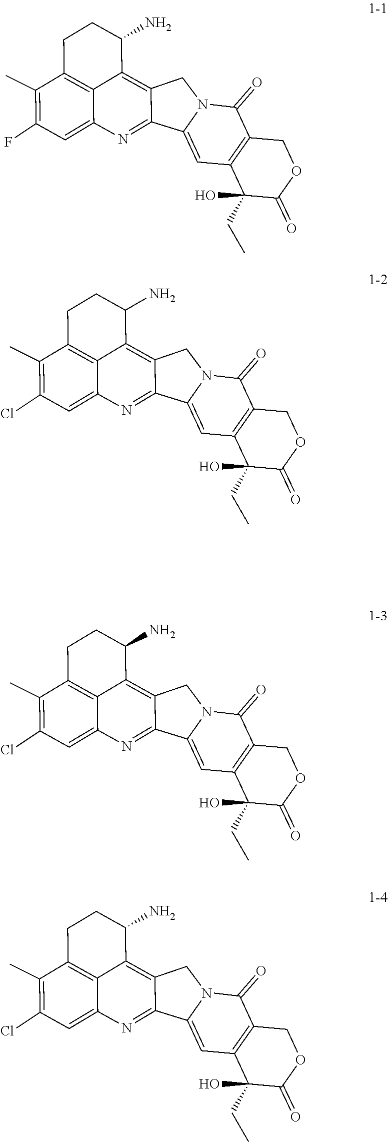 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3]

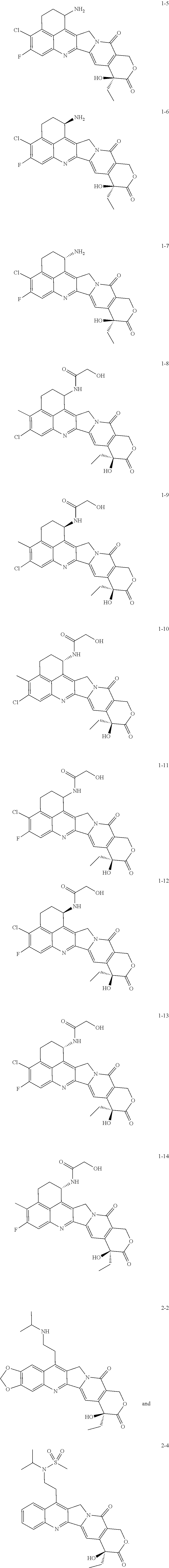

The corresponding fragment of the cytotoxic drug obtained after the cytotoxic drug is linked to the linker is D in the formula Ab-[M-L-E-D]ₓ according to the present application. In some embodiments, D is a monovalent structure obtained by the loss of one H from —OH, —NH₂, or secondary amine groups on the cytotoxic drug.

In some embodiments, D is selected from the following structures:

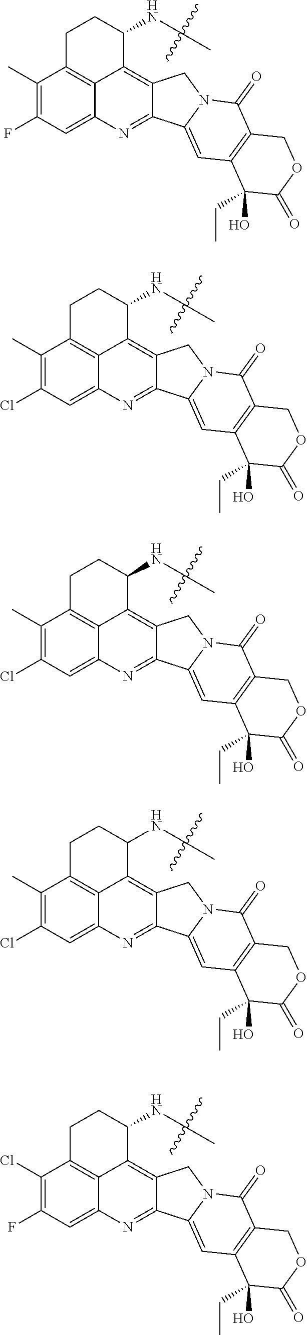 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(Cl)cc3nc4c(c1c23)Cn1c-4cc2c(c1=O)COC(=O)[C@]2(O)CC ; *N[C@@H]1CCc2c(C)c(Cl)cc3nc4c(c1c23)Cn1c-4cc2c(c1=O)COC(=O)[C@]2(O)CC ; *NC1CCc2c(C)c(Cl)cc3nc4c(c1c23)Cn1c-4cc2c(c1=O)COC(=O)[C@]2(O)CC ; *N[C@H]1CCc2c(Cl)c(F)cc3nc4c(c1c23)Cn1c-4cc2c(c1=O)COC(=O)[C@]2(O)CC]

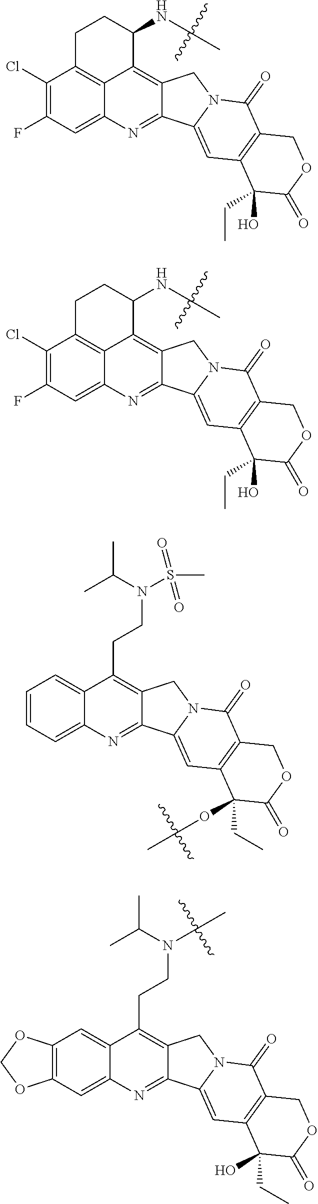 [SMILES: *N[C@@H]1CCc2c(Cl)c(F)cc3nc4c(c1c23)Cn1c-4cc2c(c1=O)COC(=O)[C@]2(O)CC ; *NC1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O ; *N(CCc1c2c(nc3cc4c(cc13)OCO4)-c1cc3c(c(=O)n1C2)COC(=O)[C@]3(O)CC)C(C)C]

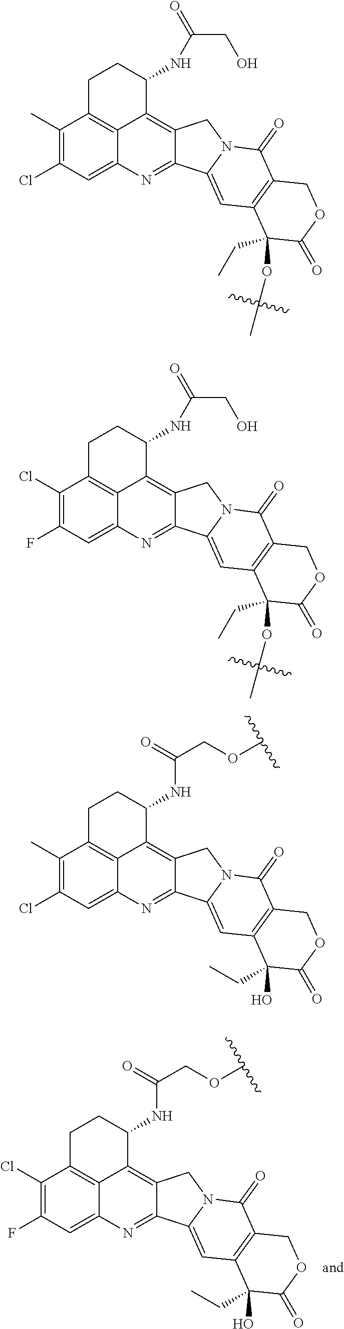 [SMILES: *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3 ; *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC]

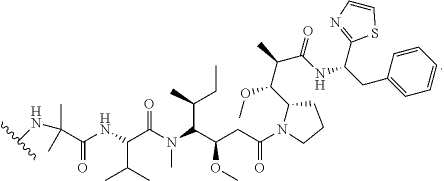

In some embodiments, the antibody-drug conjugate is selected from ADC A-01 to ADC A-26, ADC B-01 to ADC B-06, and ADC C-01 shown as follows:

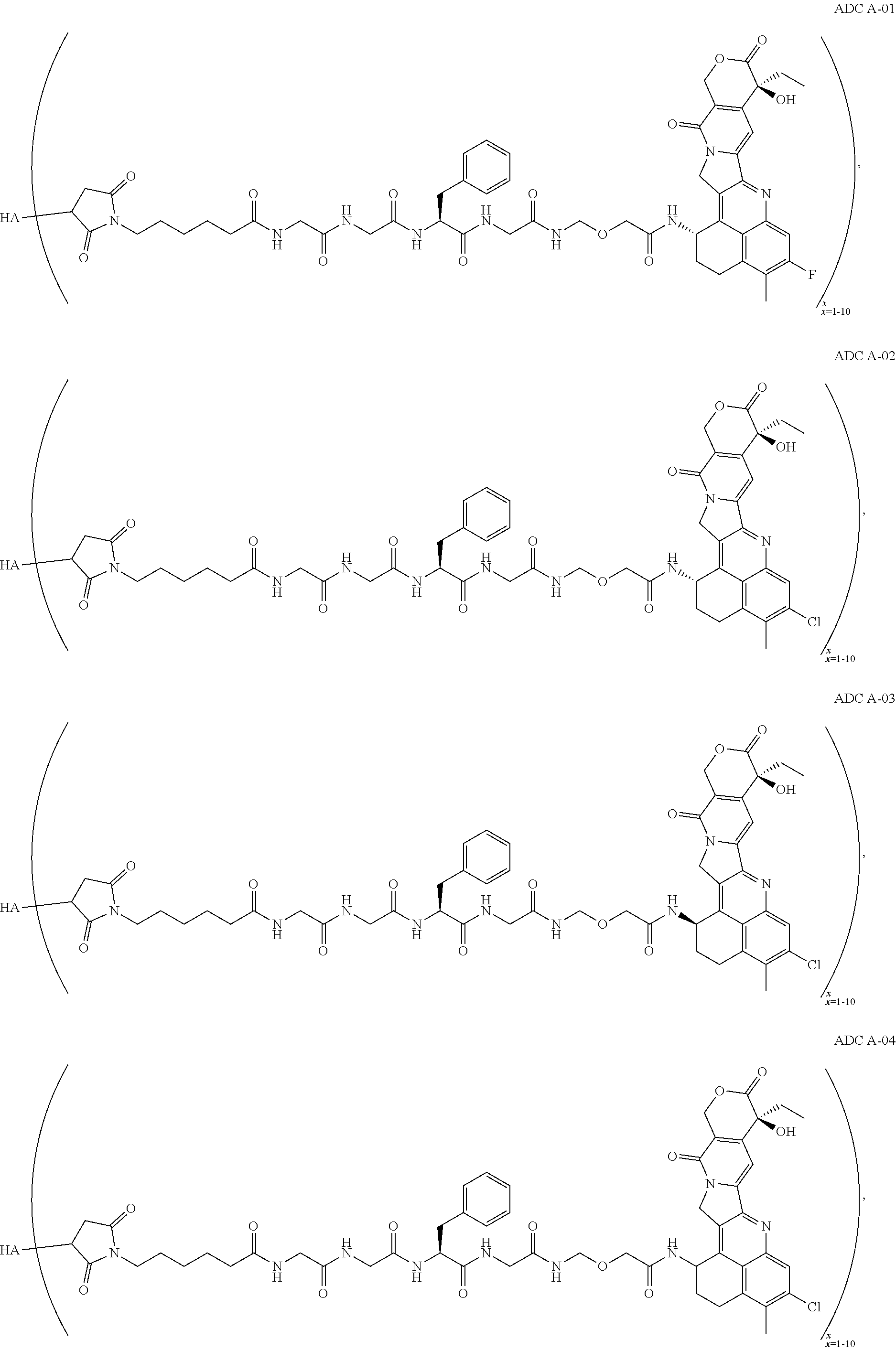

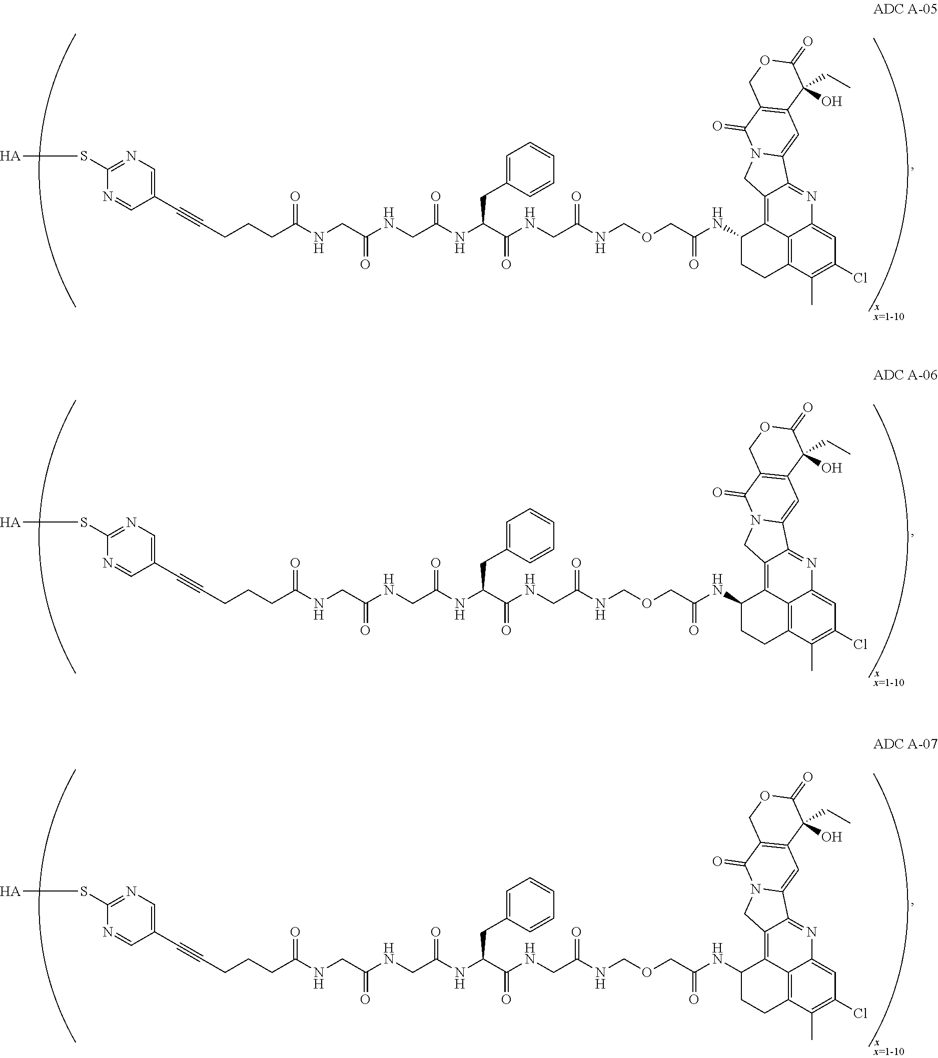

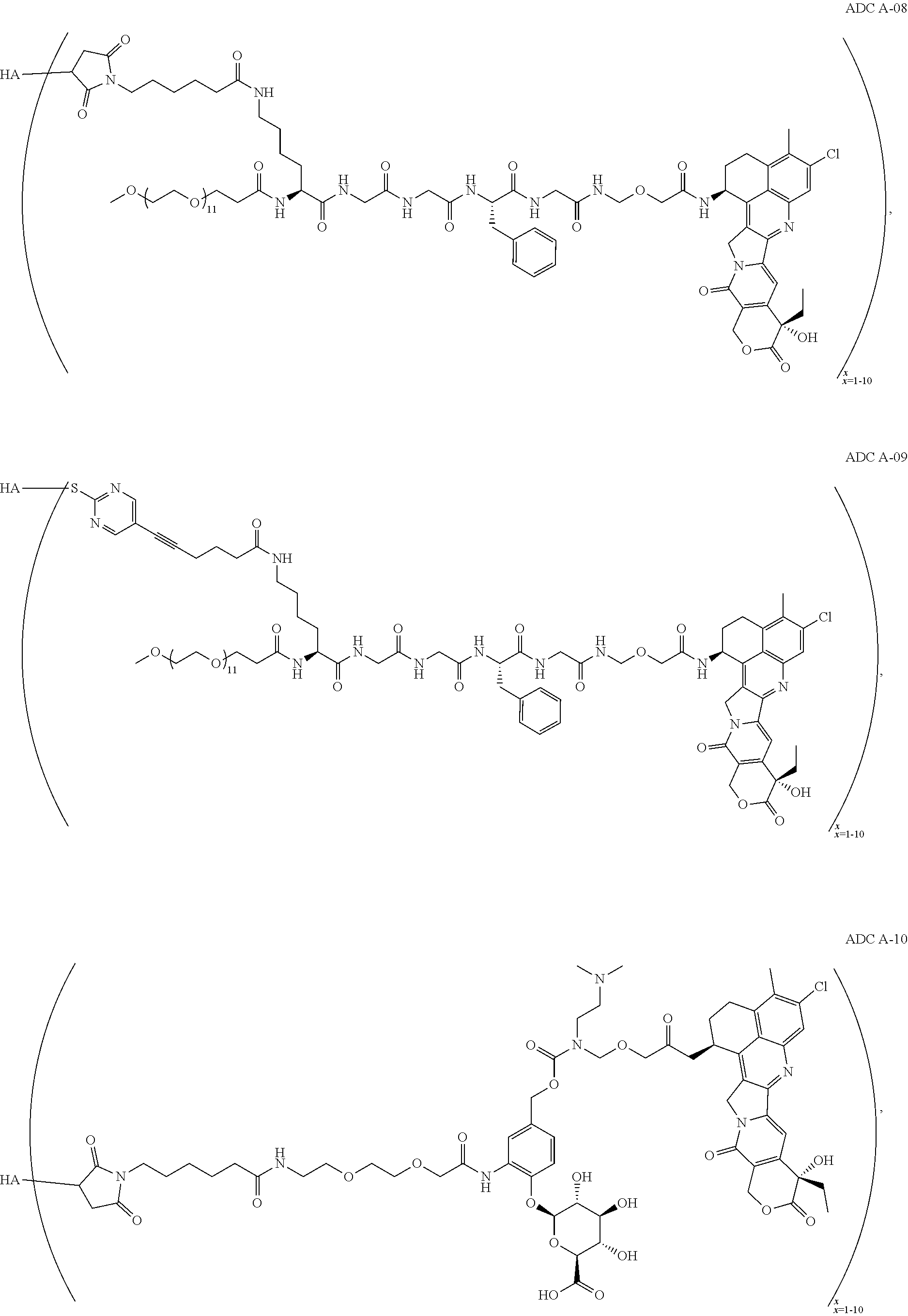

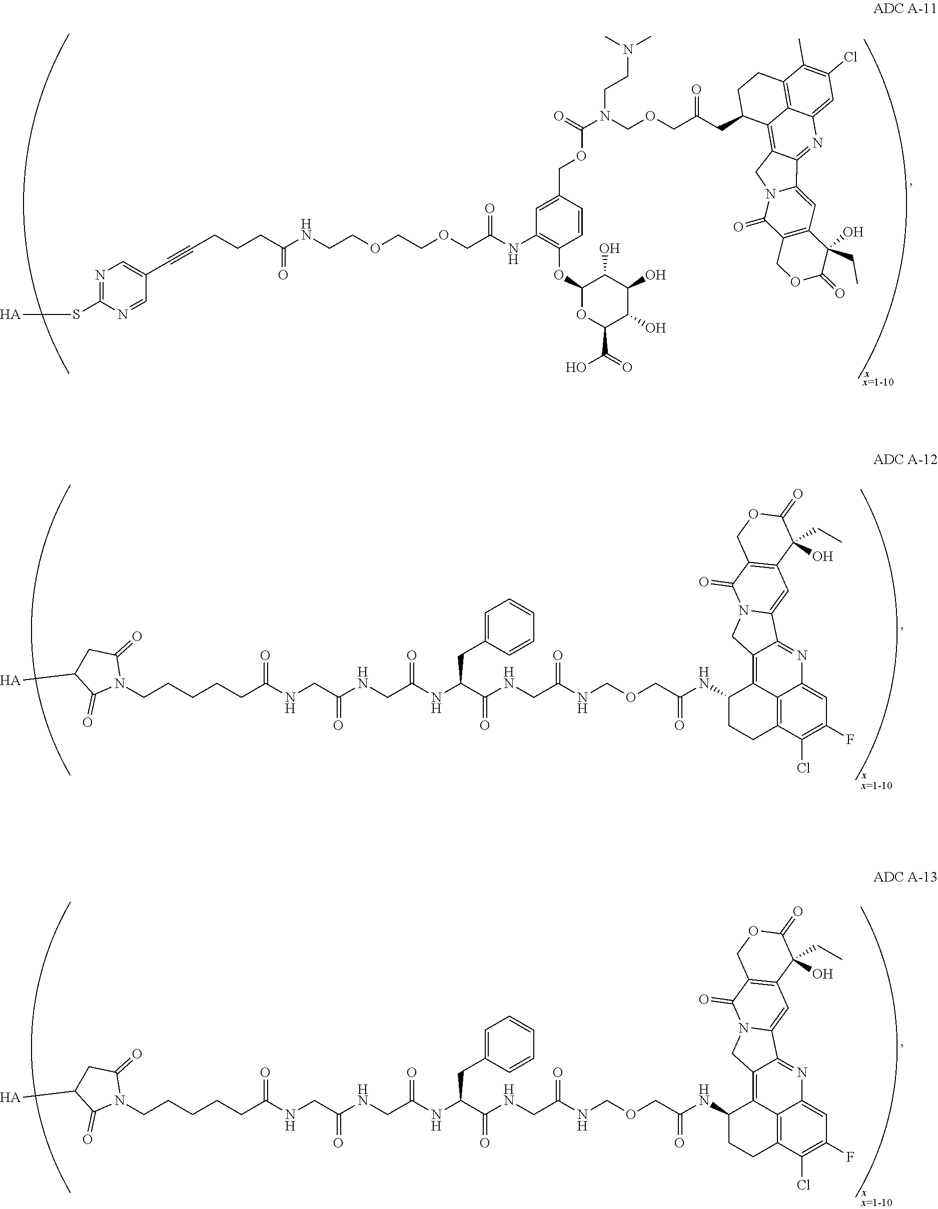

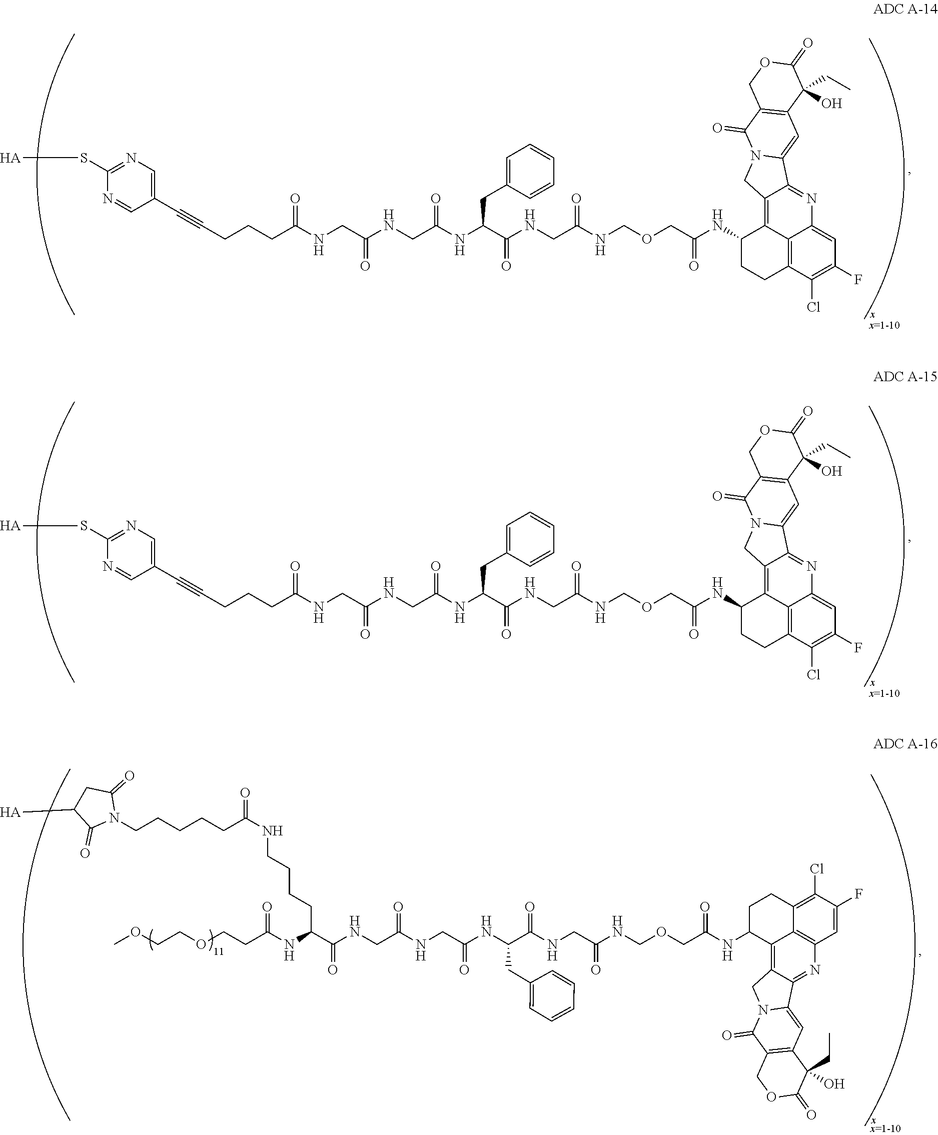

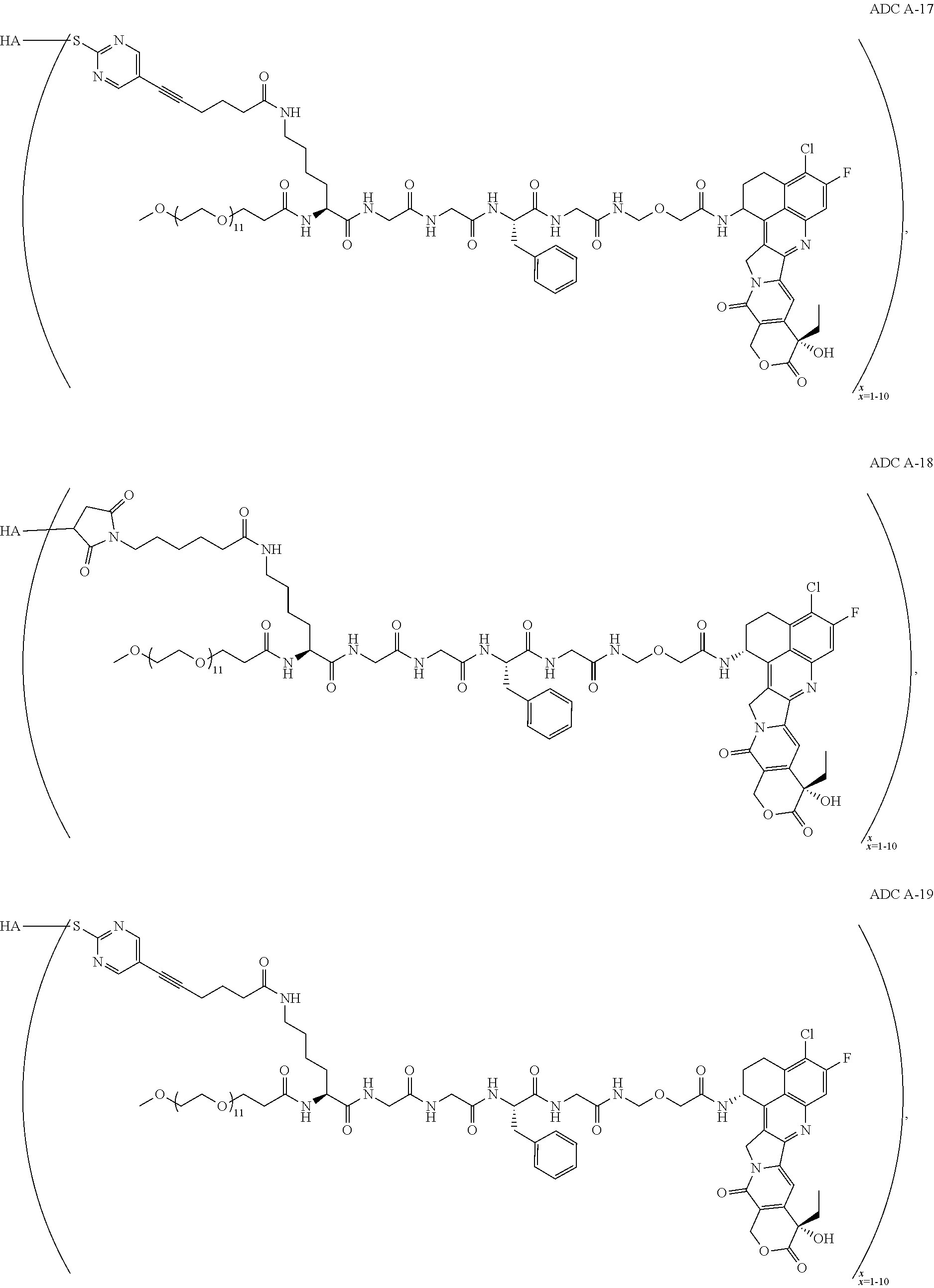

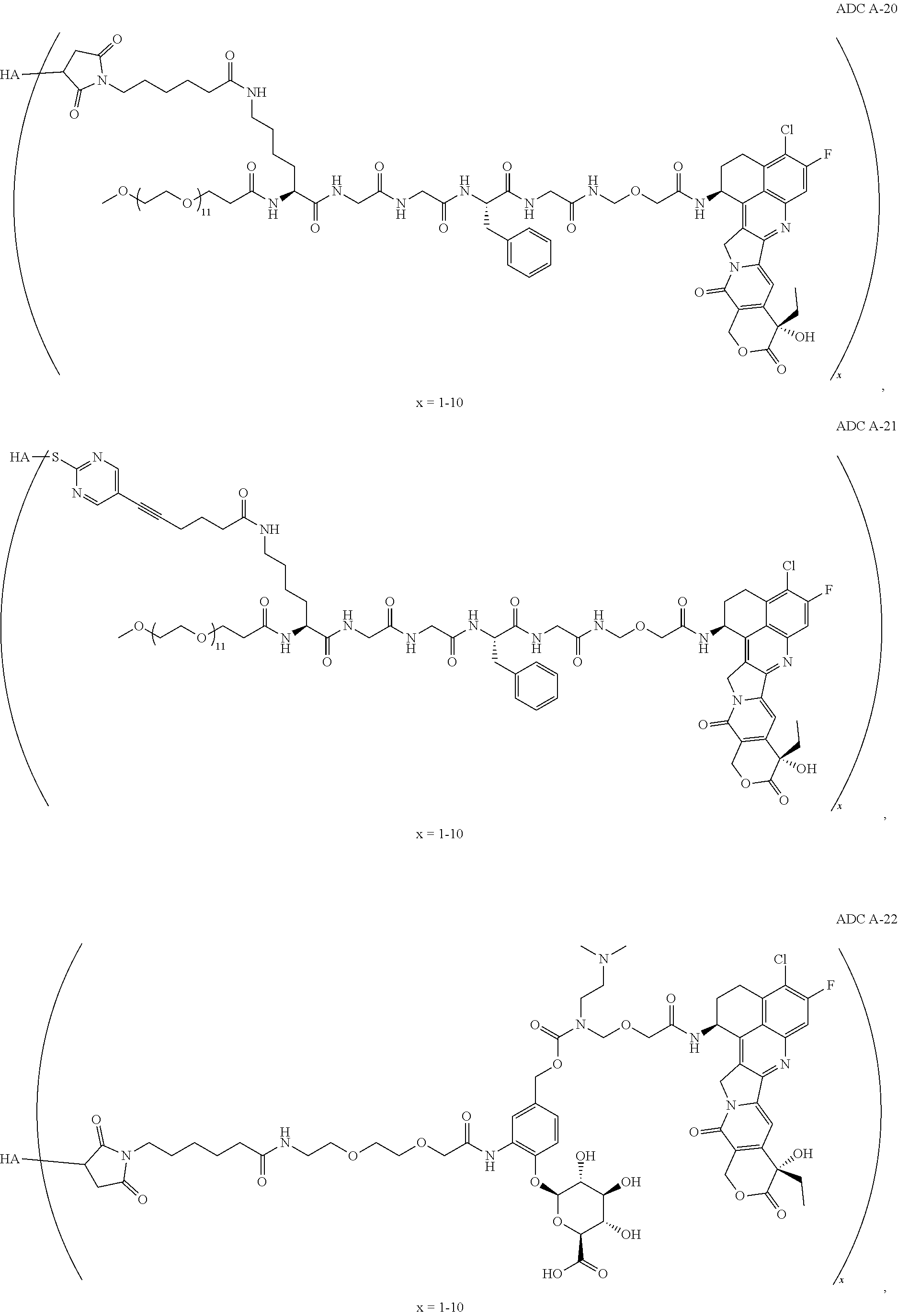

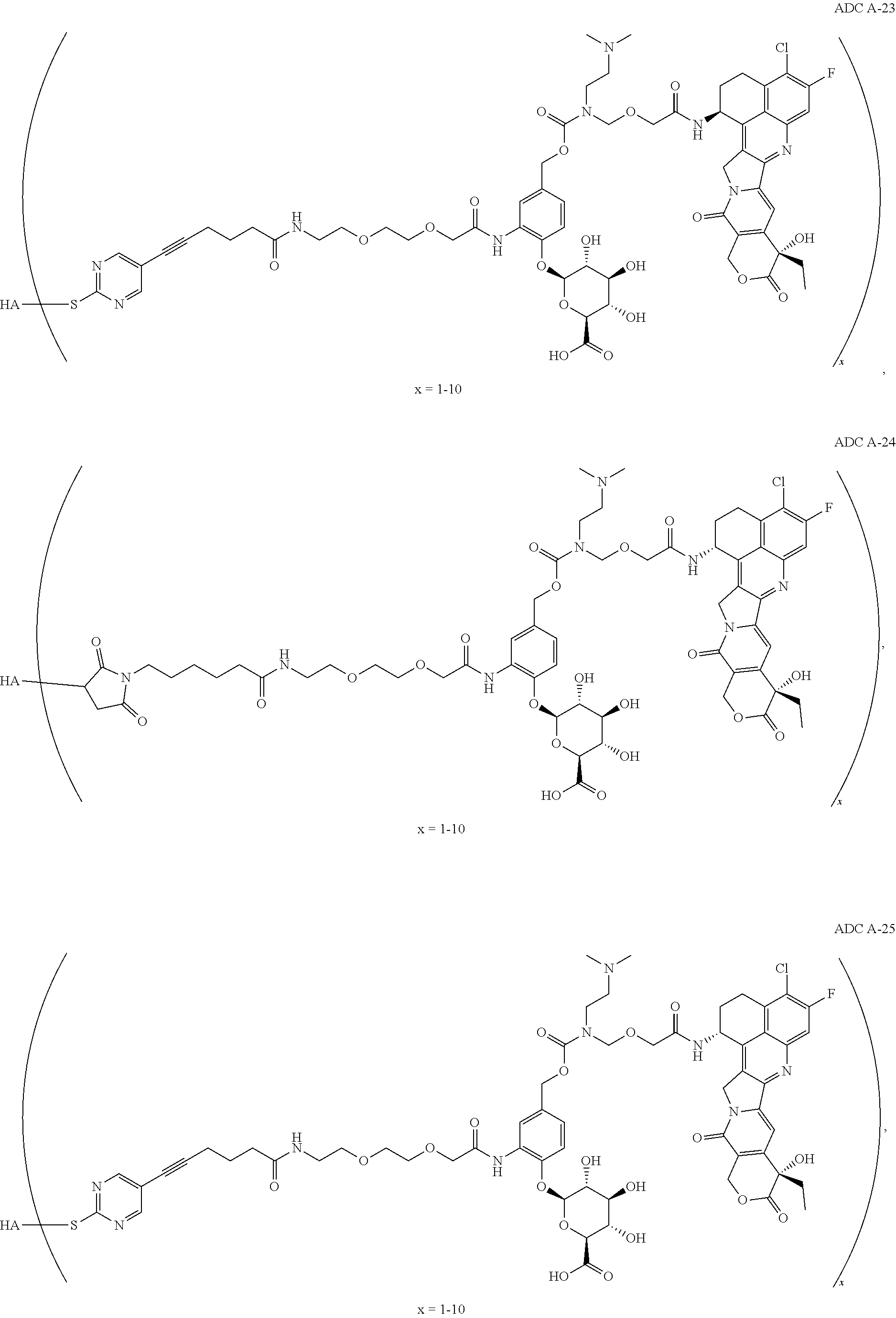

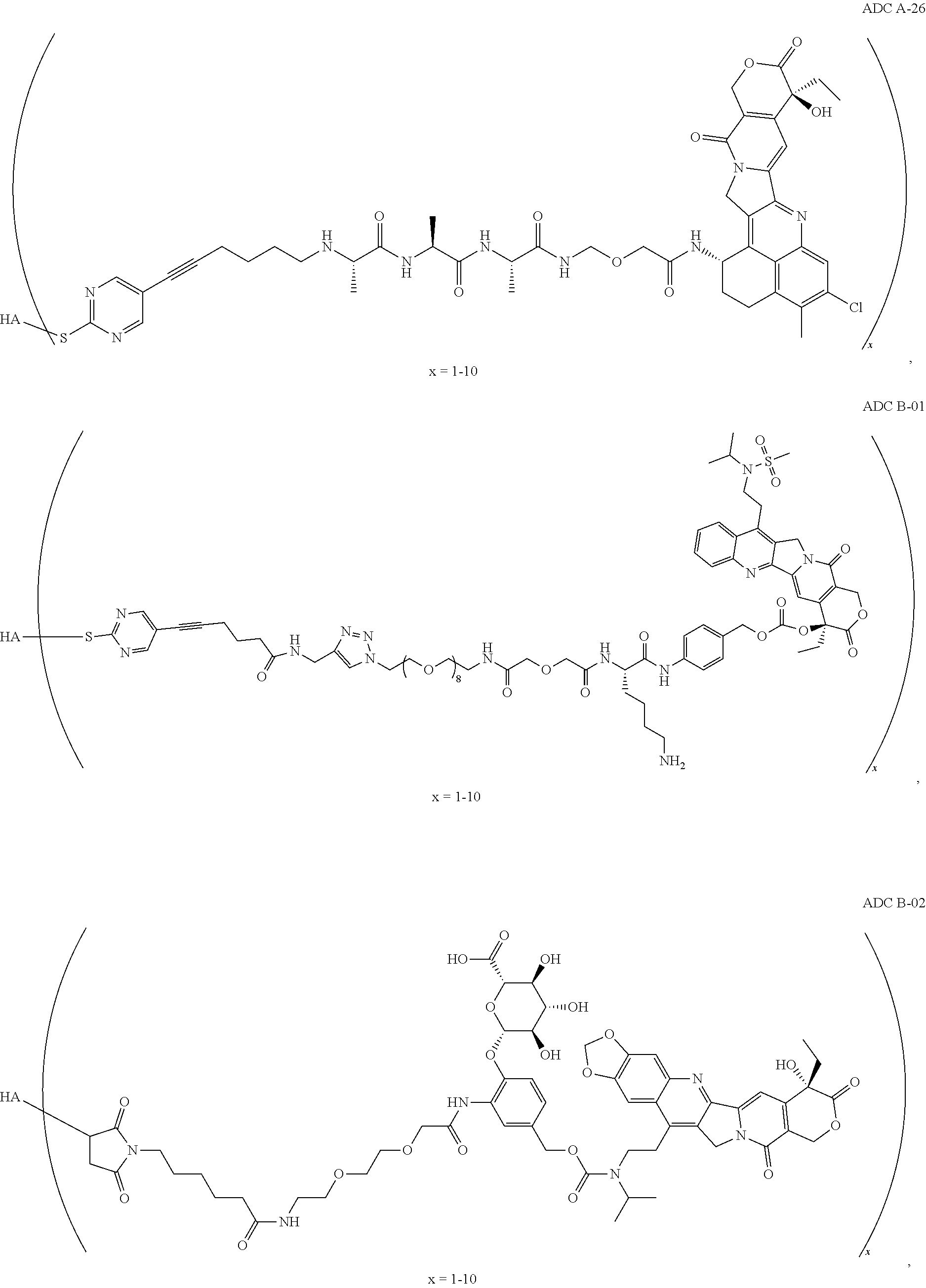

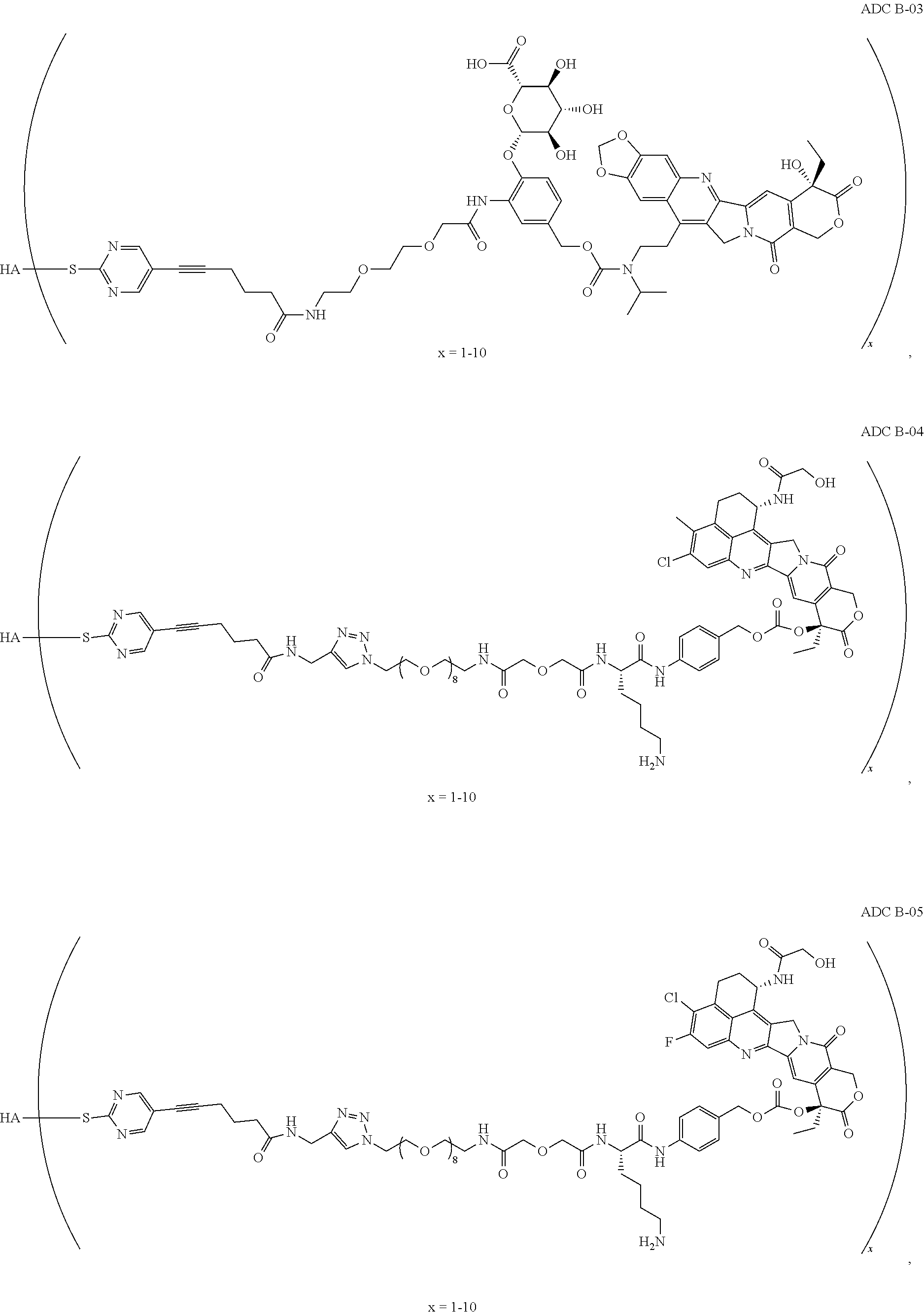

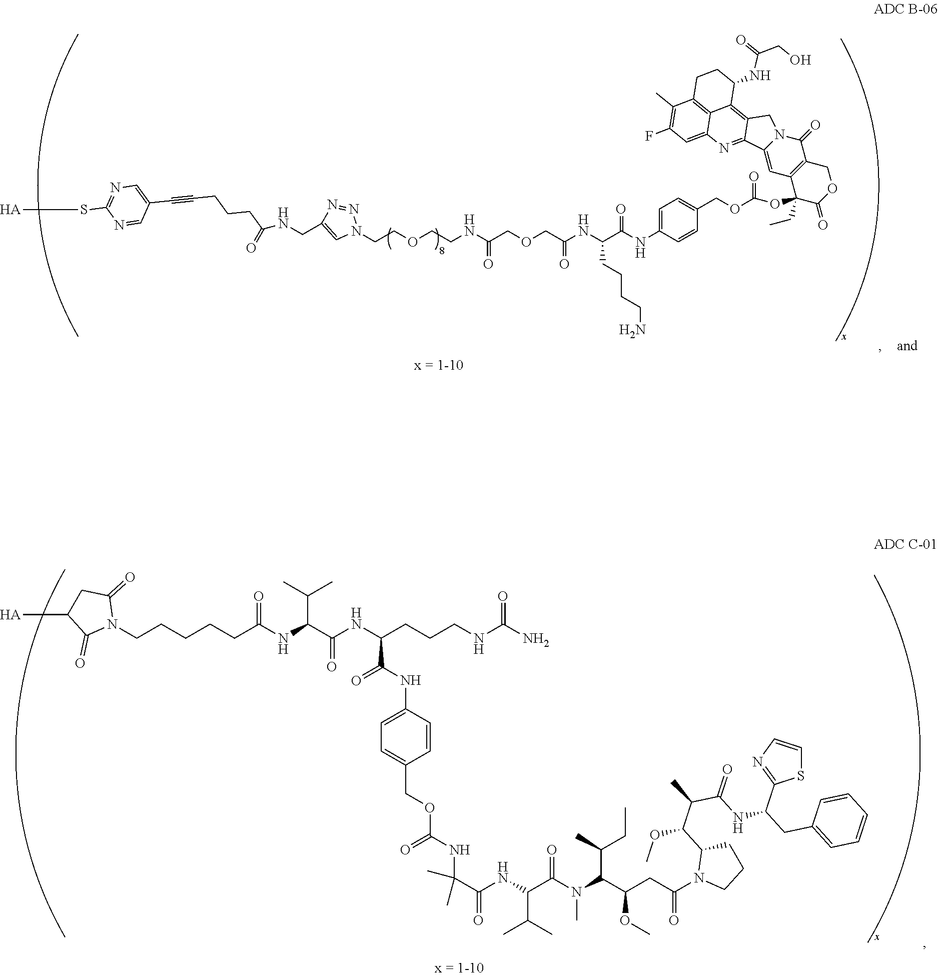

wherein HA is any one of the antibodies disclosed herein and

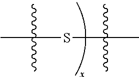 [SMILES: *S*]

represents linkage formed from a sulfhydryl in the antibody or antigen-binding fragment and the linker.

In some embodiments, the antibody-drug conjugate is selected from ADC A-01 to ADC A-25, ADC B-01 to ADC B-06, and ADC C-01 shown as follows:

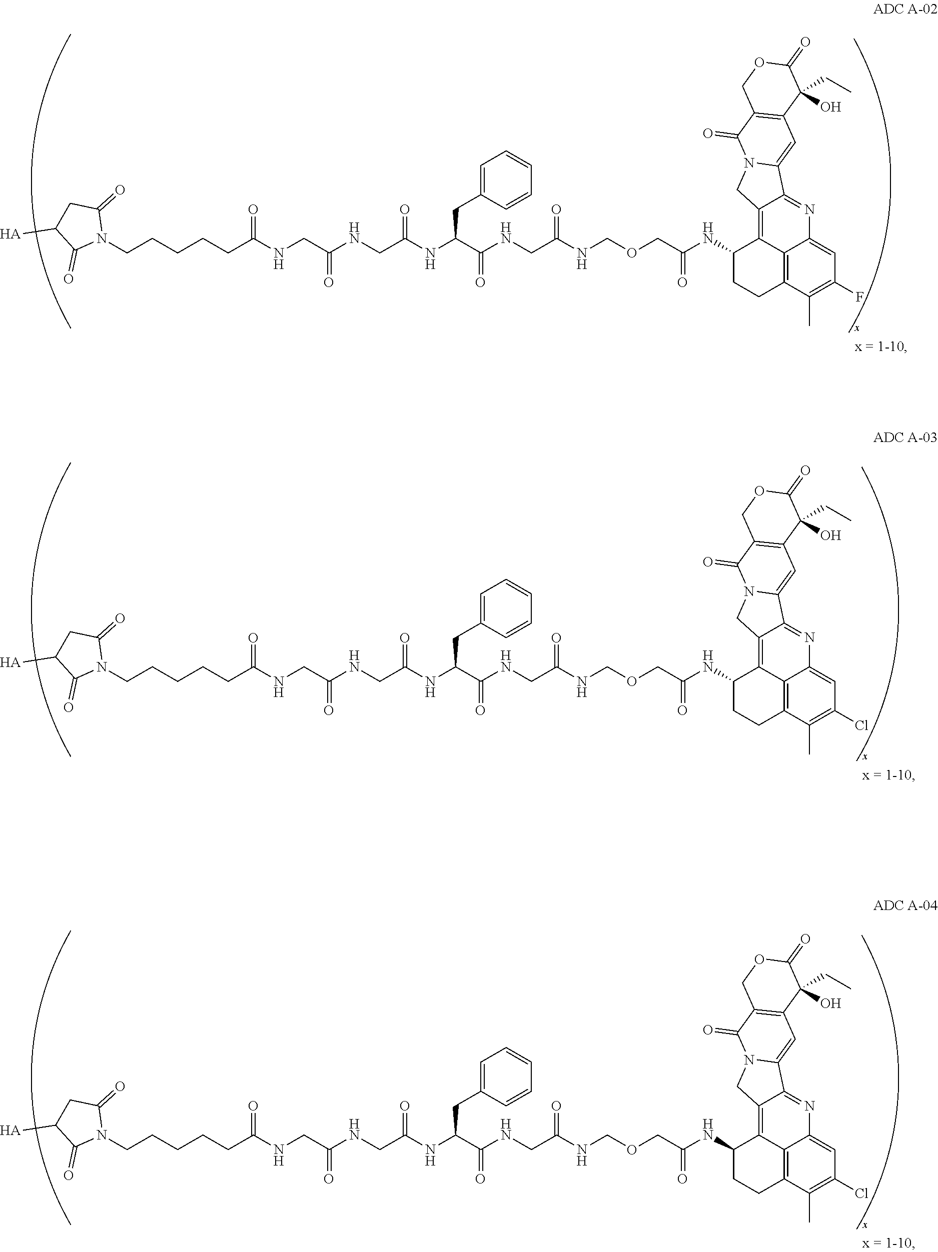

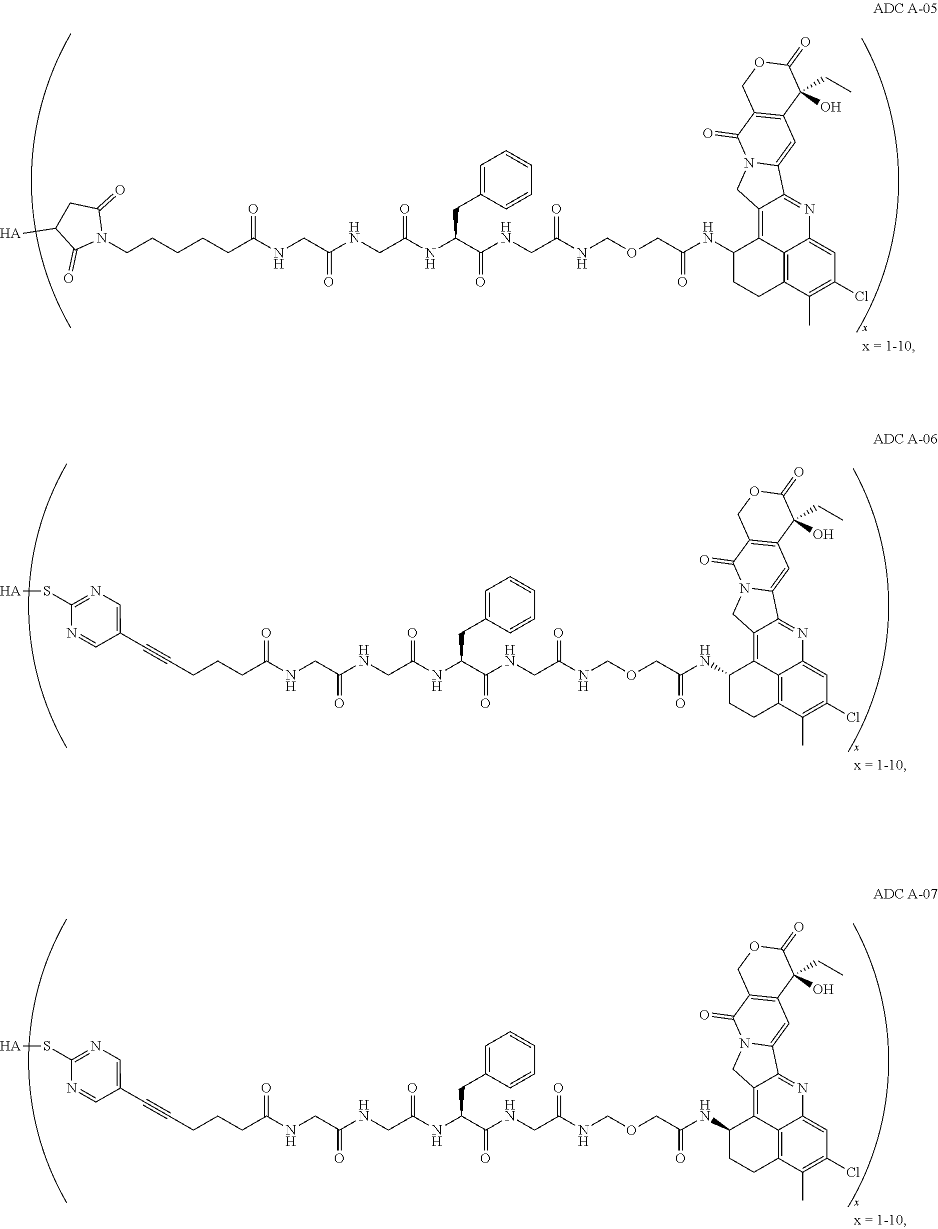

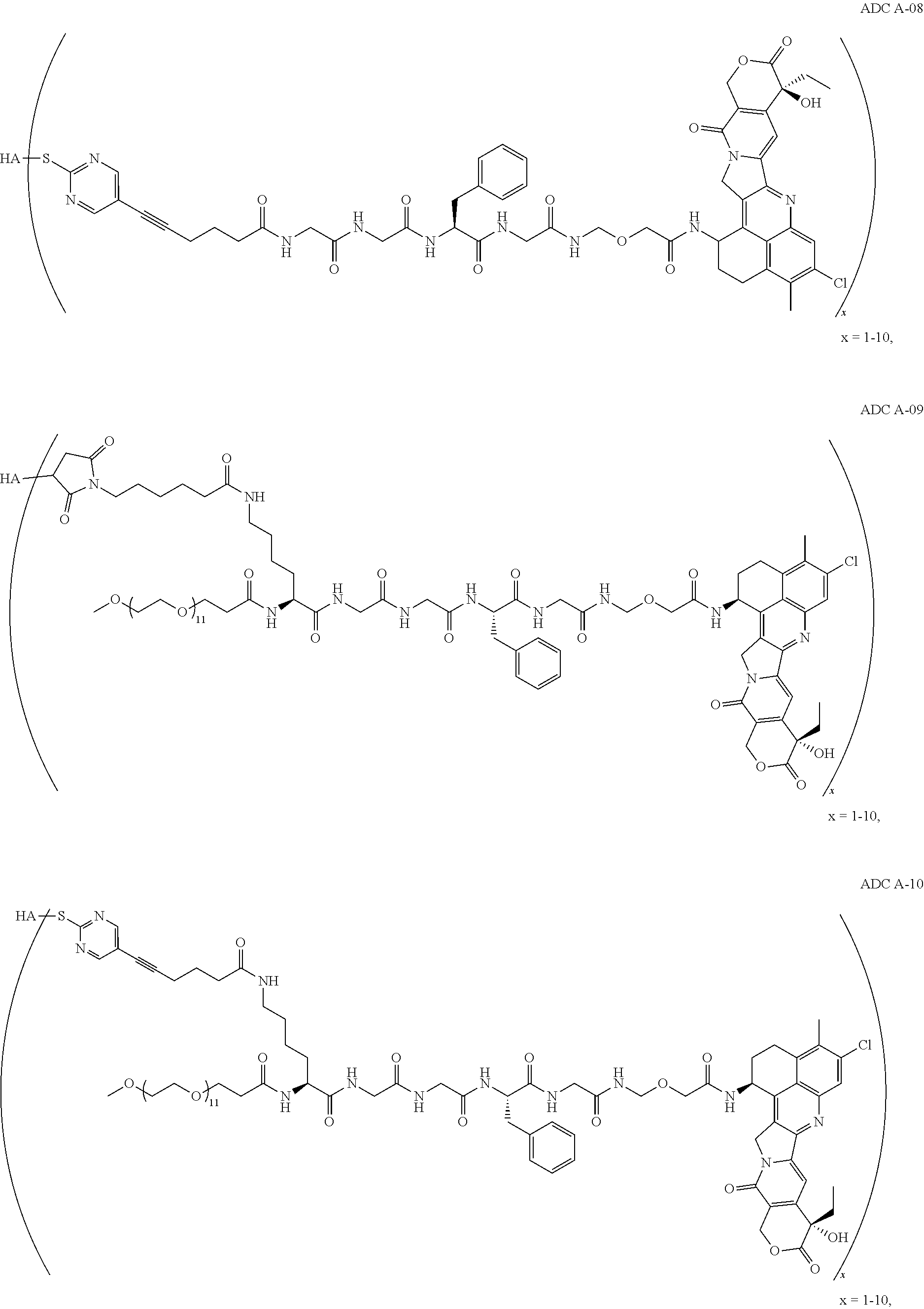

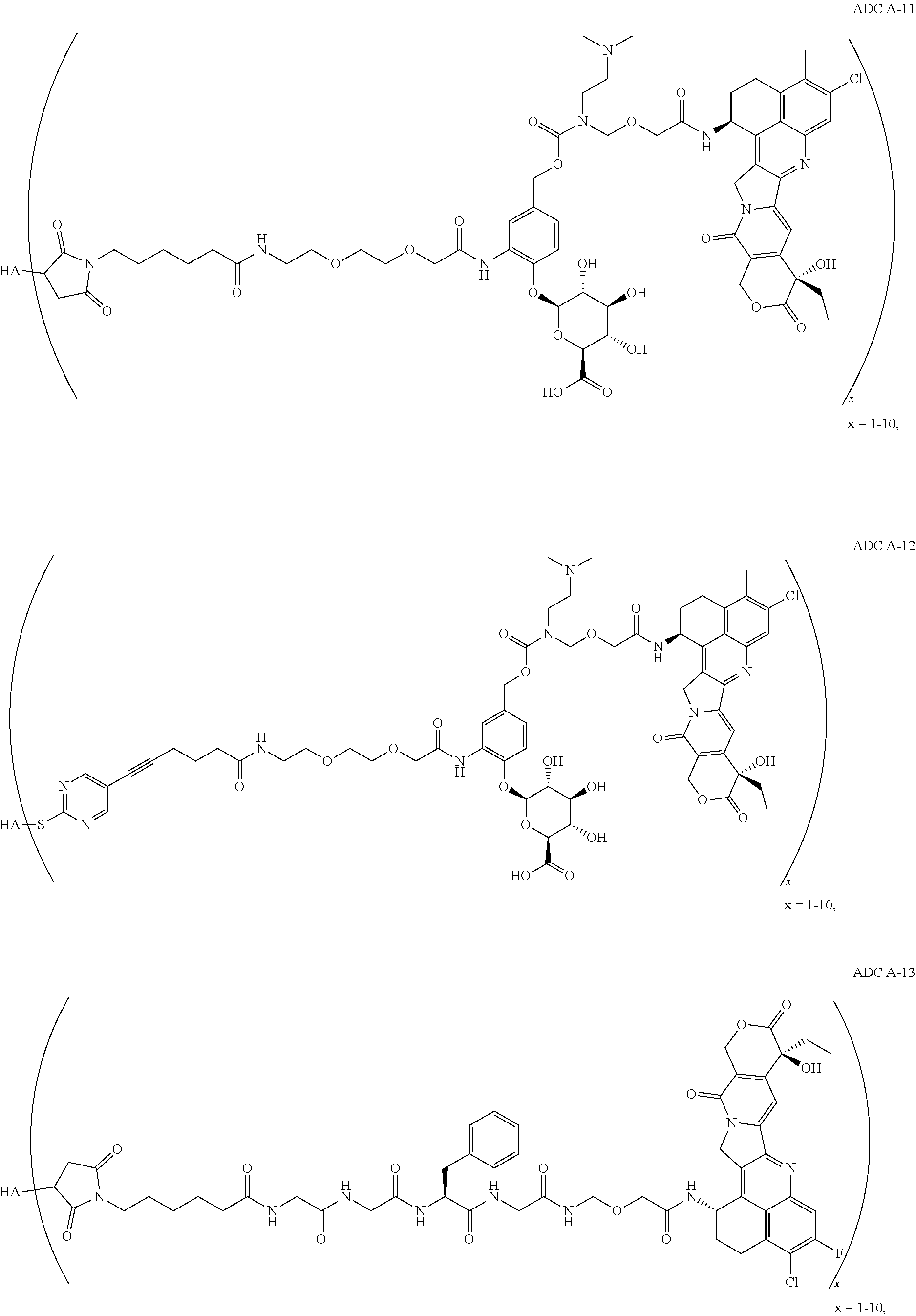

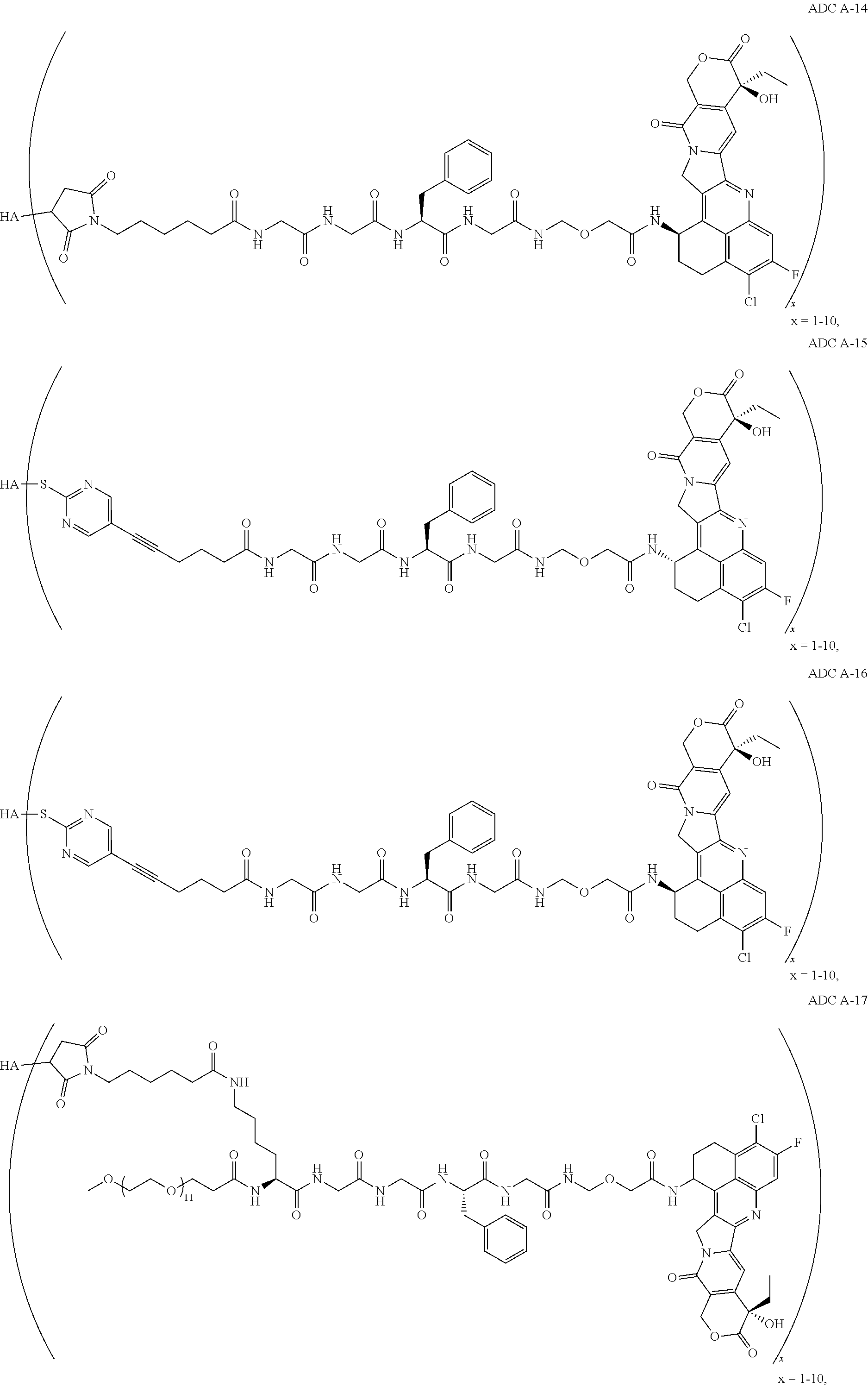

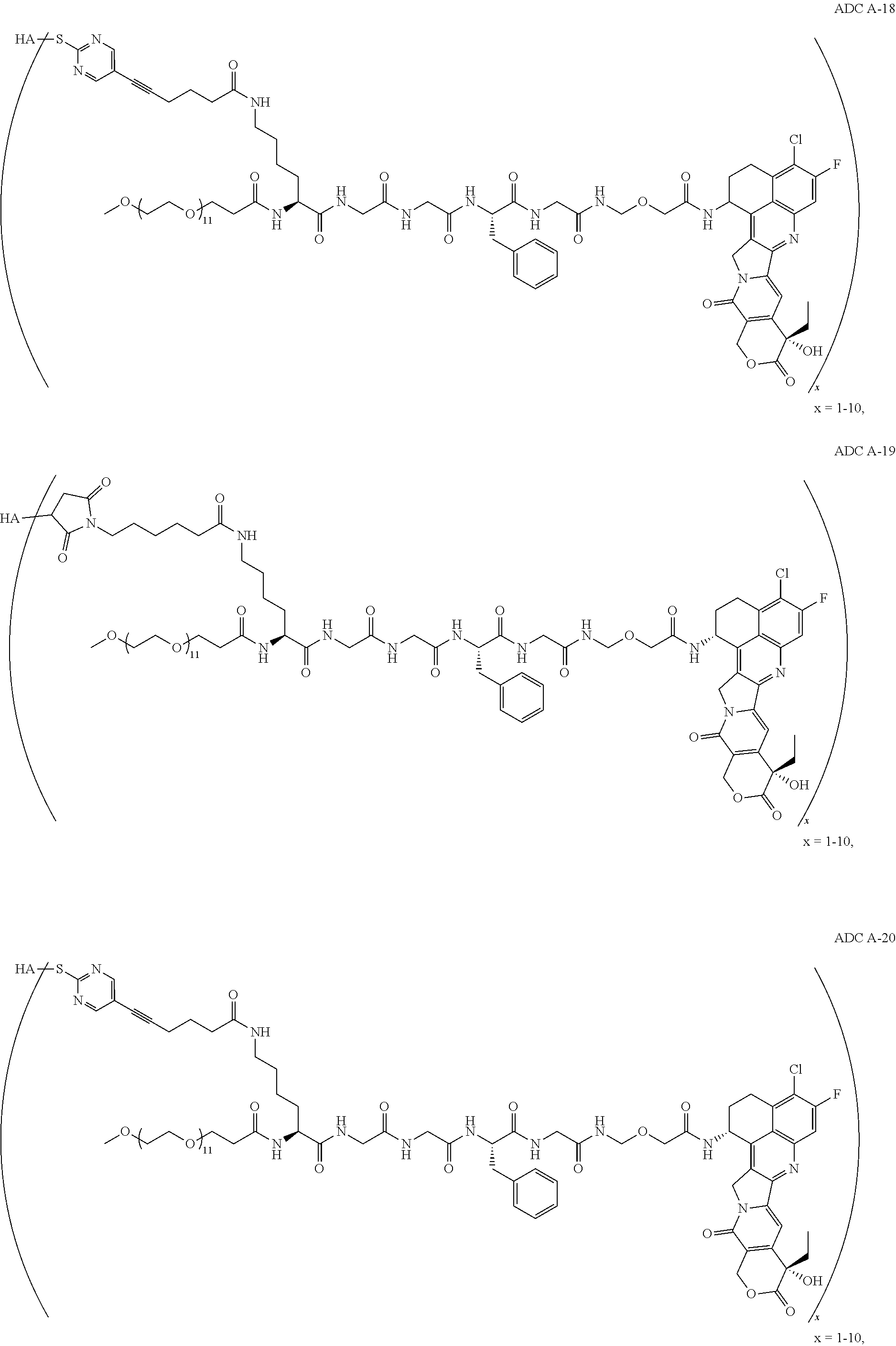

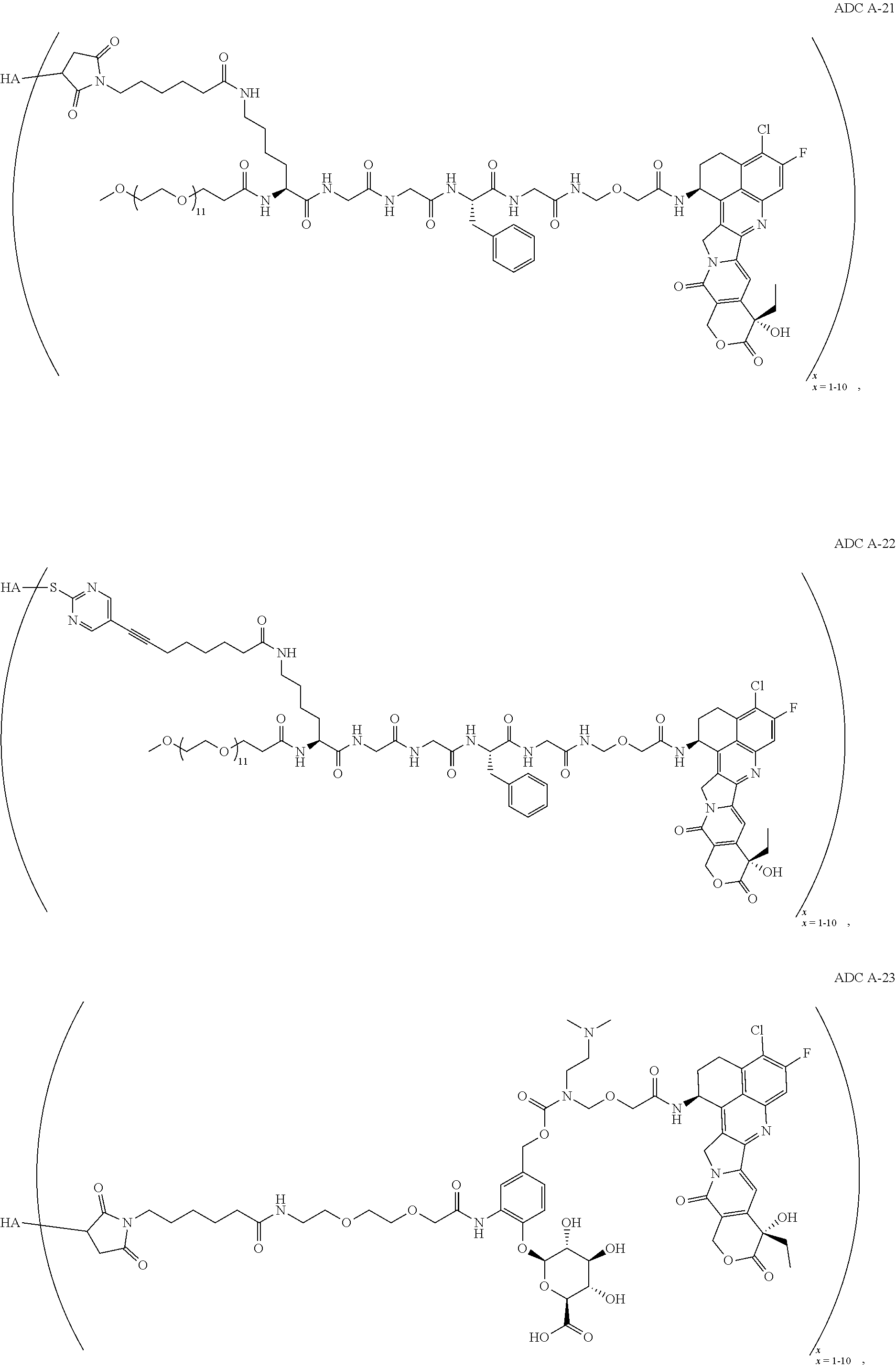

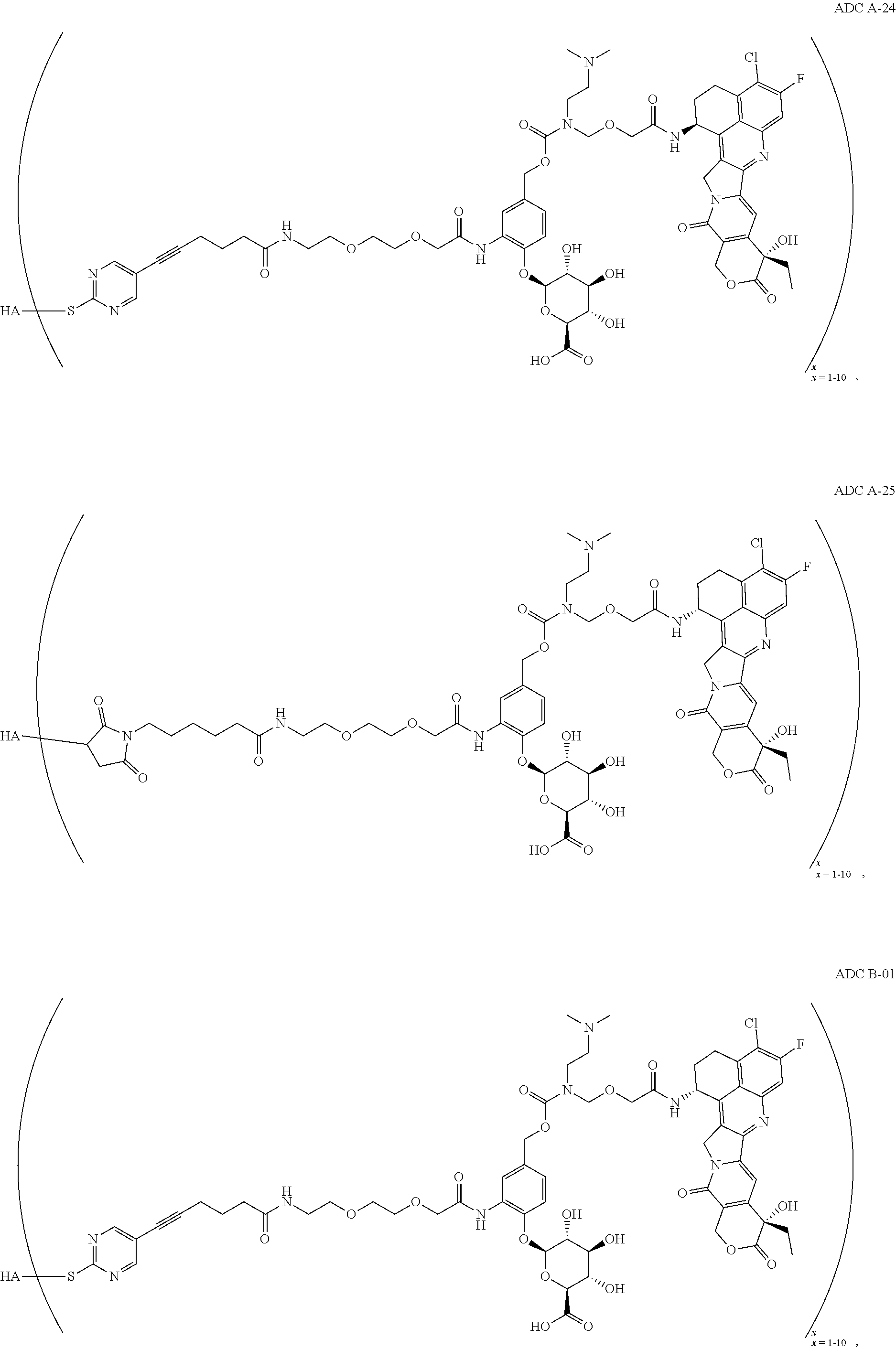

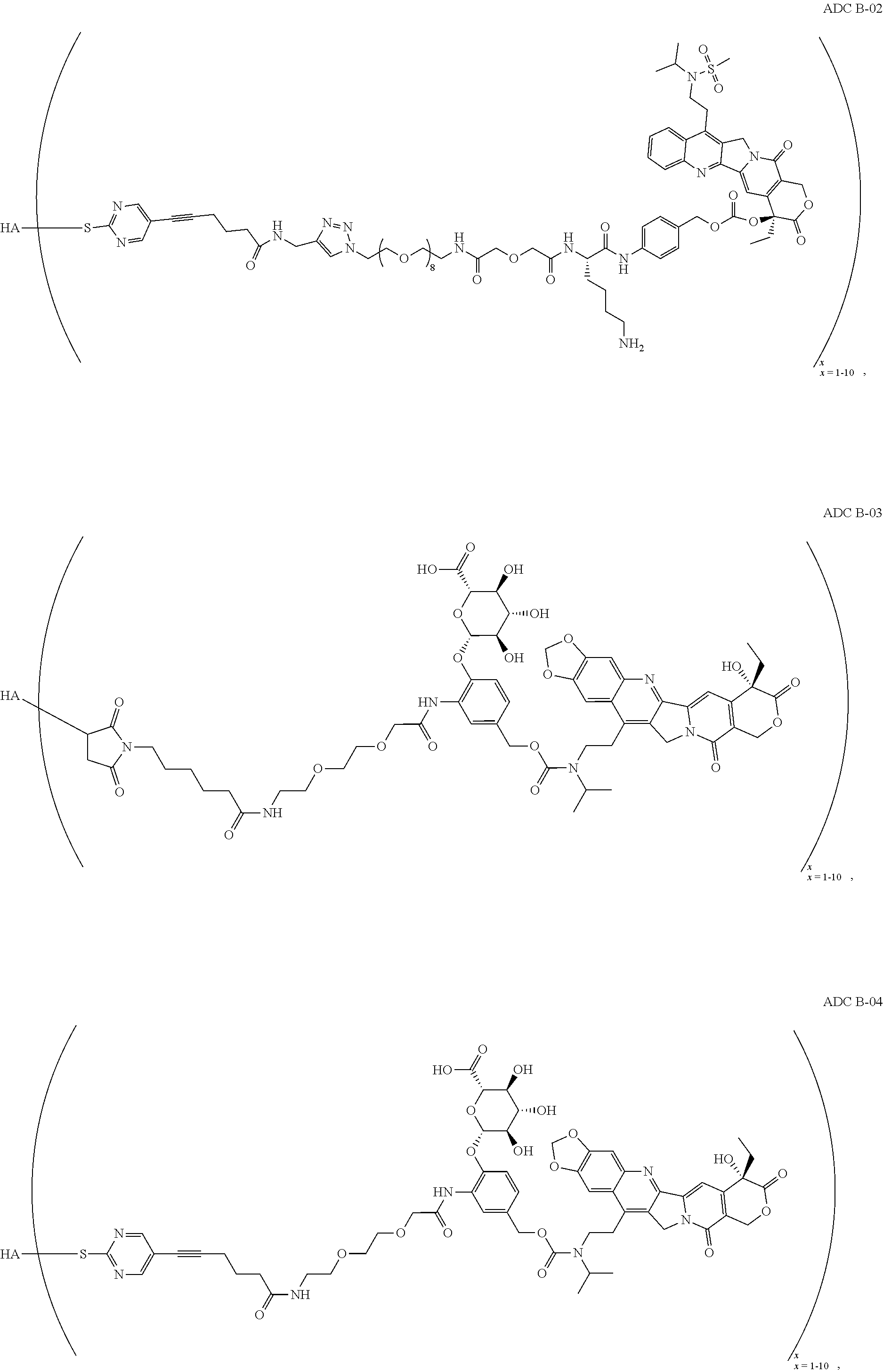

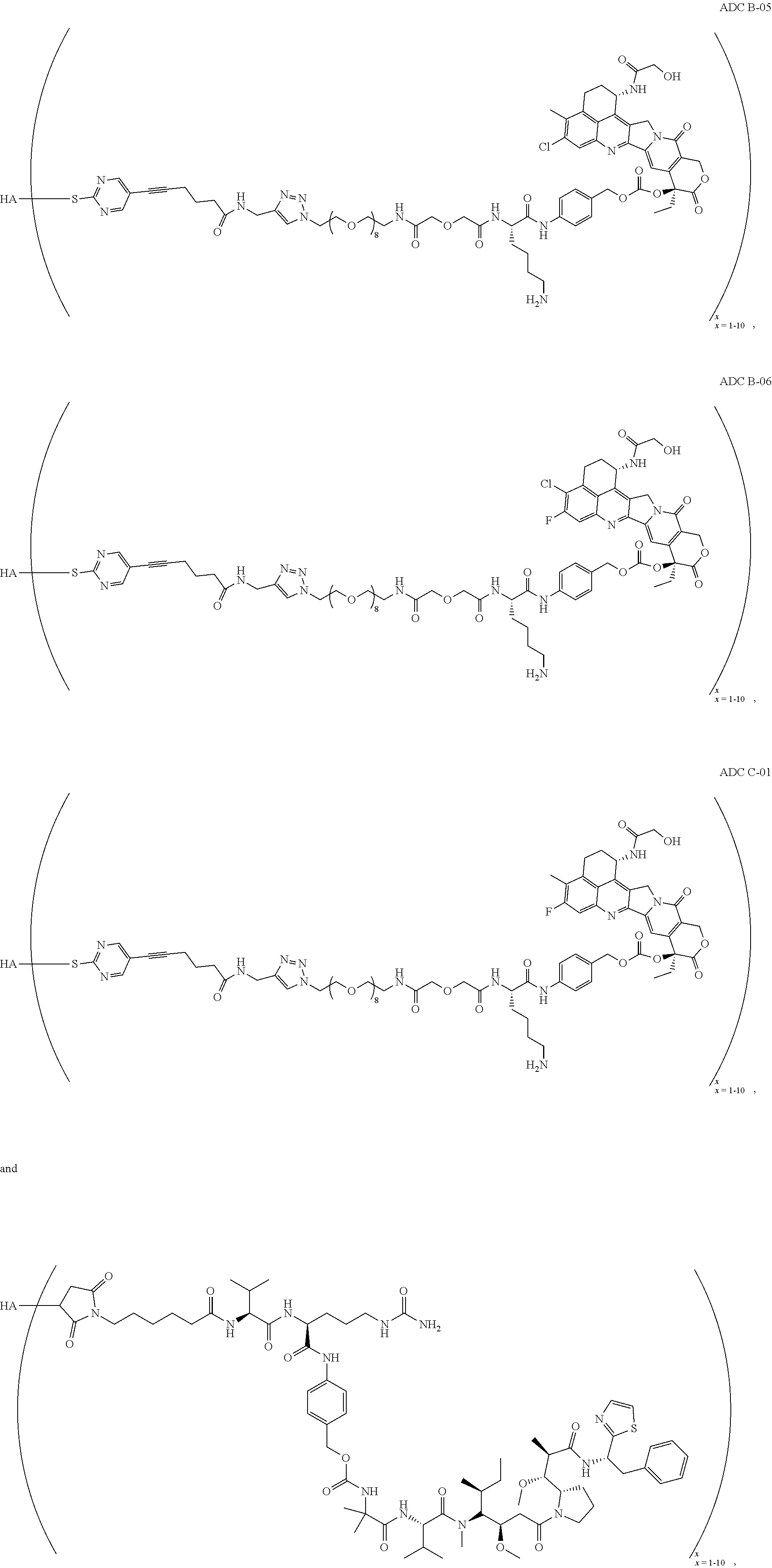

wherein HA is any one of the antibodies disclosed herein and

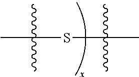 [SMILES: *S*]

represents a specific linking mode of sulfhydryl in the antibody or antigen-binding fragment and the linker.

In certain embodiments, the specific linking mode is a linkage or bond formed from a sulfhydryl in the antibody or antigen-binding fragment and the linker.

In a further embodiment, HA is an antibody comprising:
(1a) a heavy chain variable region (VH) comprising the following 3 CDRs: CDR-H1 having the amino acid sequence as set forth in SEQ ID NO: 11, CDR-H2 having the amino acid sequence as set forth in SEQ ID NO: 12, and CDR-H3 having the amino acid sequence as set forth in SEQ ID NO: 13; and/or, a light chain variable region (VL) comprising the following 3 CDRs: CDR-L1 having the amino acid sequence as set forth in SEQ ID NO: 14, CDR-L2 having the amino acid sequence as set forth in SEQ ID NO: 15, and CDR-L3 having the amino acid sequence as set forth in SEQ ID NO: 16;
(1b) a heavy chain variable region (VH) comprising the following 3 CDRs: CDR-H1 having the amino acid sequence as set forth in SEQ ID NO: 27, CDR-H2 having the amino acid sequence as set forth in SEQ ID NO: 28, CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, and CDR-L3 having the amino acid sequence as set forth in SEQ ID NO: 32;
(2a) a heavy chain variable region (VH) comprising the following 3 CDRs: CDR-H1 having the amino acid sequence as set forth in SEQ ID NO: 17, CDR-H2 having the amino acid sequence as set forth in SEQ ID NO: 18 or 19, CDR-H3 having the amino acid sequence as set forth in SEQ ID NO: 13; and/or, a light chain variable region (VL) comprising the following 3 CDRs: CDR-L1 having the amino acid sequence as set forth in SEQ ID NO: 14, CDR-L2 having the amino acid sequence as set forth in SEQ ID NO: 15, and CDR-L3 having the amino acid sequence as set forth in SEQ ID NO: 16;
(2b) a heavy chain variable region (VH) comprising the following 3 CDRs: CDR-H1 having the amino acid sequence as set forth SEQ ID NO: 33, CDR-H2 having the amino acid sequence as set forth in SEQ ID NO: 34 or 35, and CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, and CDR-L3 having the amino acid sequence as set forth in SEQ ID NO: 32;
(3a) a heavy chain variable region (VH) comprising the following 3 CDRs: CDR-H1 having the amino acid sequence as set forth in SEQ ID NO: 20, CDR-H2 having the amino acid sequence as set forth in SEQ ID NO: 21, and CDR-H3 having the amino acid sequence as set forth in SEQ ID NO: 22; and/or, a light chain variable region (VL) comprising the following 3 CDRs: CDR-L1 having the amino acid sequence as set forth in SEQ ID NO: 23, CDR-L2 having the amino acid sequence as set forth in SEQ ID NO: 24, and CDR-L3 having the amino acid sequence as set forth in SEQ ID NO: 16;
(3b) a heavy chain variable region (VH) comprising the following 3 CDRs: CDR-H1 having the amino acid sequence as set forth in SEQ ID NO: 36, CDR-H2 having the amino acid sequence as set forth in SEQ ID NO: 37, and CDR-H3 having the amino acid sequence as set forth in SEQ ID NO: 38; and/or, a light chain variable region (VL) comprising the following 3 CDRs: CDR-L1 having the amino acid sequence as set forth in SEQ ID NO: 39, CDR-L2 having the amino acid sequence as set forth in SEQ ID NO: 40, and CDR-L3 having the amino acid sequence as set forth in SEQ ID NO: 32;
(4a) a heavy chain variable region (VH) comprising the following 3 CDRs: CDR-H1 having the amino acid sequence as set forth in SEQ ID NO: 25, CDR-H2 having the amino acid sequence as set forth in SEQ ID NO: 26, and CDR-H3 having the amino acid sequence as set forth in SEQ ID NO: 13; and/or, a light chain variable region (VL) comprising the following 3 CDRs: CDR-L1 having the amino acid sequence as set forth in SEQ ID NO: 14, CDR-L2 having the amino acid sequence as set forth in SEQ ID NO: 15, and CDR-L3 having the amino acid sequence as set forth in SEQ ID NO: 16; or,
(4b) a heavy chain variable region (VH) comprising the following 3 CDRs: CDR-H1 having the amino acid sequence as set forth in SEQ ID NO: 41, CDR-H2 having the amino acid sequence as set forth in SEQ ID NO: 42, and CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, and CDR-L3 having the amino acid sequence as set forth in SEQ ID NO: 32.

In a further embodiment, HA comprises: a VH having the amino acid sequence as set forth in SEQ ID NO: 1, and/or, a VL having the amino acid sequence as set forth in SEQ ID NO: 2.

In a further embodiment, HA comprises: a VH having the amino acid sequence as set forth in SEQ ID NO: 3, and/or, a VL having the amino acid sequence as set forth in SEQ ID NO: 4.

In a further embodiment, HA comprises: a VH having the amino acid sequence as set forth in SEQ ID NO: 5, and/or, a VL having the amino acid sequence as set forth in SEQ ID NO: 6.

In a further embodiment, HA comprises: a VH having the amino acid sequence as set forth in SEQ ID NO: 7, and/or, a VL having the amino acid sequence as set forth in SEQ ID NO: 8.

In a further embodiment, HA comprises: a VH having the amino acid sequence as set forth in SEQ ID NO: 9, and/or, a VL having the amino acid sequence as set forth in SEQ ID NO: 10.

In a further embodiment, HA comprises: (1) a heavy chain comprising a VH having the amino acid sequence as set forth in SEQ ID NO: 1 and a heavy chain constant region (CH) having the amino acid sequence as set forth in SEQ ID NO: 43, and, a light chain comprising a VL having the amino acid sequence as set forth in SEQ ID NO: 2 and a light chain constant region (CL) having the amino acid sequence as set forth in SEQ ID NO: 44.

In a further embodiment, HA comprises: (2) a heavy chain comprising a VH having the amino acid sequence as set forth in SEQ ID NO: 3 and a heavy chain constant region (CH) having the amino acid sequence as set forth in SEQ ID NO: 43 or 45, and, a light chain comprising a VL having the amino acid sequence as set forth in SEQ ID NO: 4 and a light chain constant region (CL) having the amino acid sequence as set forth in SEQ ID NO: 44.

In a further embodiment, HA comprises: (3) a heavy chain comprising a VH having the amino acid sequence as set forth in SEQ ID NO: 5 and heavy chain constant region (CH) having the amino acid sequence as set forth in SEQ ID NO: 43, and, a light chain comprising a VL having the amino acid sequence as set forth in SEQ ID NO: 6 and light chain constant region (CL) having the amino acid sequence as set forth in SEQ ID NO: 44.

In a further embodiment, HA comprises: (4) a heavy chain comprising a VH having the amino acid sequence as set forth in SEQ ID NO: 7 and heavy chain constant region (CH) having the amino acid sequence as set forth in SEQ ID NO: 43, and, a light chain comprising a VL having the amino acid sequence as set forth in sequence SEQ ID NO: 8 and light chain constant region (CL) having the amino acid sequence as set forth in SEQ ID NO: 44.

In a further embodiment, HA comprises: (5) a heavy chain comprising a VH having the amino acid sequence as set forth in SEQ ID NO: 9 and heavy chain constant region (CH) having the amino acid sequence as set forth in SEQ ID NO: 43, and, a light chain comprising a VL sequence having the amino acid sequence as set forth in SEQ ID NO: 10 and light chain constant region (CL) having the amino acid sequence as set forth in SEQ ID NO: 44.

In a further embodiment, HA comprises: a heavy chain (HC) having the amino acid sequence as set forth in SEQ ID NO: 46, and comprises a light chain (LC) having the amino acid sequence as set forth in SEQ ID NO: 47.

In some embodiments, the antibody or antigen-binding fragment thereof further comprises:
(a) a human immunoglobulin heavy chain constant region (CH) or a variant thereof, wherein the variant has replacement, deletion or addition of one or more amino acids (for example, replacement, deletion or addition of up to 20, up to 15, up to 10 or up to 5 amino acids; for example, replacement, deletion or addition of 1, 2, 3, 4 or 5 amino acids) as compared to a wild-type sequence from which the variant is derived; and
(b) a human immunoglobulin light chain constant (CL) or a variant, wherein the variant has replacement, deletion or addition of one or more amino acids (for example replacement, deletion or addition of up to 20, up to 15, up to 10 or up to 5 amino acids; for example, replacement, deletion or addition of 1, 2, 3, 4 or 5 amino acids) as compared to the wild-type sequence from which the variant is derived.

In one embodiment, HA in each antibody-drug conjugate represents antibodies or antigen-binding fragments of VH shown as SEQ ID NO: 1, 3, 5, 7 or 9 and VL shown as SEQ ID NO: 2, 4, 6, 8 or 10, for example the antibodies or antigen-binding fragments of VH shown as SEQ ID NO: 3 and CH shown as SEQ ID NO: 43 or 45, VL shown as SEQ ID NO: 4 and CL shown as SEQ ID NO: 44; and

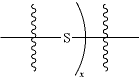 [SMILES: *S*]

represents linkage formed from a sulfhydryl in the antibody or antigen-binding fragment and the linker.

In particular embodiments of HA or antibody-drug conjugate disclosed herein, the heavy chain constant domains may comprise a C-terminal lysine or lack either a C-terminal lysine or a C-terminal glycine-lysine dipeptide. In some embodiments of the antibody or antigen binding fragment thereof, the N-terminal amino acid of the antibody or antigen binding fragment thereof may undergo cyclization to pyroglutamate.

In some embodiments of the antibody or antigen binding fragment thereof, the N-terminal amino acid of the antibody or antigen binding fragment thereof may undergo cyclization to pyroglutamatic acid.

Thus, a composition may comprise a population of antibody-drug conjugate species wherein each species may independently comprise a C-terminal lysine, lack a C-terminal lysine, lack a C-terminal glycine-lysine and/or comprise an N-terminal glutamine or glutamic acid or cyclization of the N-terminal amino acid to pyroglutamate.

In certain embodiments, the composition may comprise a population of antibody-drug conjugate species wherein each species may independently comprise a C-terminal lysine, lack a C-terminal lysine, lack a C-terminal glycine-lysine and/or comprise an N-terminal glutamine or glutamic acid or cyclization of the N-terminal amino acid to pyroglutamatic acid.

Therefore, in particular embodiments, the present invention further provides compositions comprising an ADC disclosed herein wherein the predominant ADC species in the composition comprises (i) antibodies in which the heavy chain C-terminus lacks a lysine residue; (ii) antibodies in which the heavy chain N-terminus is glutamine, glutamic acid, or pyroglutamate; or, (iii) antibodies in which the heavy chain C-terminus lacks a lysine residue and the heavy chain N-terminus is glutamine, glutamic acid, or pyroglutamate.

In particular embodiments, the present invention further provides compositions comprising an ADC disclosed herein wherein the predominant ADC species in the composition comprises (i) antibodies in which the heavy chain C-terminus lacks a lysine residue; (ii) antibodies in which the heavy chain N-terminus is glutamine, glutamic acid, or pyroglutamic acid; or, (iii) antibodies in which the heavy chain C-terminus lacks a lysine residue and the heavy chain N-terminus is glutamine, glutamic acid, or pyroglutamic acid.

As known to those skilled in the art, pyroglutamic acid is the conjugate acid of pyroglutamate, and is in equilibrium with pyroglutamate in solution.

### Compositions

In another aspect, the present application provides a composition of antibody-drug conjugates (ADCs) as described herein. Such composition may comprise a plurality of ADCs as described herein, wherein each ADC comprises a drug-linker as described herein. Therefore, a composition may be characterized by a “drug-to-antibody” ratio (DAR) ranging from about 1 to about 10. Methods to determine DAR are well known to the skilled person and include methods using Reverse Phase Chromatography, or HPLC-MS.

In some embodiments, the DAR value (drug-antibody conjugation ratio) of the composition of drug-antibody conjugate is 1-10, for example: 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10, preferably 1-8, preferably 3-9, for example, 3.0-3.5, 3.0-4.0, 3.0-4.5, 3.0-5.0, 3.0-5.5, 3.0-6.0, 3.5-4.0, 3.5-4.5, 3.5-5.0, 3.5-5.5, 3.5-6.0, 3.5-6.5, 3.5-7.0, 3.5-7.5, 3.5-8.0, 4.0-4.5, 4.0-5.0, 4.0-5.5, 4.0-6.0, 4.0-6.5, 4.0-7.0, 4.0-7.5, 4.0-8.0, 4.5-5.0, 4.5-5.5, 4.5-6.0, 4.5-6.5, 4.5-7.0, 4.5-7.5, 4.5-8.0, 5.0-5.5, 5.0-6.0, 5.0-6.5, 5.0-7.0, 5.0-7.5, 5.0-8.0, 5.5-6.0, 5.5-6.5, 5.5-7.0, 5.5-7.5, 5.5-8.0, 6.0-6.5, 6.0-7.0, 6.0-7.5, 6.0-8.5, 6.5-7.0, 6.5-7.5, 6.5-8.5, 7.0-7.5, 7.0-9.0 or 7.5-9.0.

In one embodiment, the DAR value is 8. In one embodiment, the DAR value is 7 to 9. In one embodiment, the DAR value is 7 to 8. In other embodiments, the DAR value of a composition of antibody drug conjugate comprising ADC A-05 is about 8. In other embodiments, the DAR value of a composition of antibody drug conjugate comprising ADC A-05 is 7 to 9. In other embodiments, the DAR value of a composition of antibody drug conjugate comprising ADC A-05 is 7 to 8. In other embodiments, the DAR value of a composition of antibody drug conjugate comprising ADC B-01 is 7 to 8. In other embodiments, the DAR value of a composition of antibody drug conjugate comprising ADC C-01 is 2 to 4. In other embodiments, the DAR value of a composition of antibody drug conjugate comprising ADC B-04 is 7 to 9. In other embodiments, the DAR value of a composition of antibody drug conjugate comprising ADC A-26 is 7 to 9.

### Drug-Linker

Those skilled in the art should be understood that the antibody-drug conjugate described in the present application can be prepared in a modularized manner. For example, the “drug-linker” in a free form (which can be understood as M′-L-E-D, wherein M′ is a structural form before covalent linkage of M to the antibody or antigen binding fragment thereof) is obtained, and then the ‘drug-linker’ in the free form is covalently linked to the antibody or antigen binding fragment thereof to obtain the antibody-drug conjugate according to the present application. Correspondingly, M′ in the free form of ‘drug-linker’ is linked to one or more sulfhydryl (—SH), amino (—NH₂) or carboxyl (—COOH) on the antibody or antigen binding fragment thereof through a substitution reaction (for example, removal of —SO₂Me or —Br and the like structures thereon) or through an addition reaction and other manners.

In another aspect, the present invention provides a drug-linker having a structure shown as the formula M′-L-E-D, where

M′ is

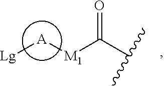

and Lg is a leaving group for a nucleophilic substitution reaction (for example, halogen, methanesulfonyl, fluorophenol, or

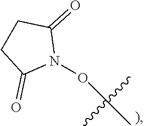 [SMILES: *ON1C(=O)CCC1=O]

or hydroxyl (—OH), sulfhydryl (—SH) or amino (—NH₂); or, Lg forms an unsaturated double bond with an adjacent atom on ring A; ring A is a 5-6 membered aliphatic heterocyclic ring, or a 5-20 membered aromatic ring system, and the aliphatic heterocyclic ring and the aromatic ring system are optionally substituted by one or more groups selected from oxo (═O), halogen, cyano, amino, carboxyl, sulfhydryl and C₁₋₆ alkyl; and M₁ is selected from a single bond, C₁₋₂₀ alkylene, C₂₋₂₀ alkenylene and C₂₋₂₀ alkynylene; and

L, E and D structures are defined according to any one of above antibody-drug conjugate.

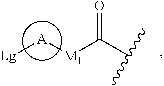

In some embodiments, M′ is Lg is methanesulfonyl, or Lg and adjacent atoms on the ring A form carbon-carbon double bonds; the ring A is a 5-membered alicyclic heterocyclic ring, a 6-membered heteroaromatic ring, or a polycyclic ring formed by linking more than one 6-membered heteroaromatic ring and a benzene ring through a single bond, and the alicyclic heterocyclic ring is optionally substituted by one or more groups selected from oxo (═O), halogen and C₁₋₄ alkyl; and M₁ is selected from a single bond, C₃₋₁₀ alkylene, C₃₋₁₀ alkenylene and C₃₋₁₀ alkynylene.

In some embodiments, M′ is

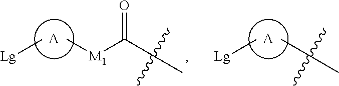

is selected from

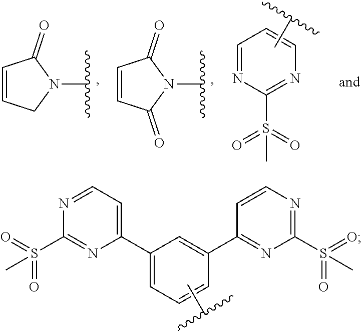

and M₁ is selected from a single bond, C₅₋₈ alkylene, C₅₋₈ alkenylene and C₅₋₈ alkynylene.

In some embodiments, M′ is selected from

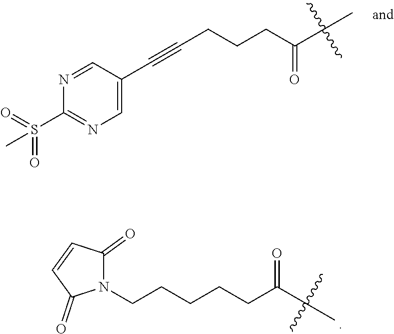 [SMILES: *C(=O)CCCC#Cc1cnc(S(C)(=O)=O)nc1 ; *C(=O)CCCCCN1C(=O)C=CC1=O]

In some embodiments, M′ is

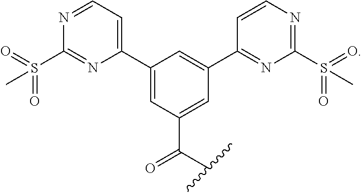 [SMILES: *C(=O)c1cc(-c2ccnc(S(C)(=O)=O)n2)cc(-c2ccnc(S(C)(=O)=O)n2)c1]

In some embodiments, the “drug-linker” in free form is selected from A-01 to A-26, B-01 to B-06, and C-01 shown as follows:

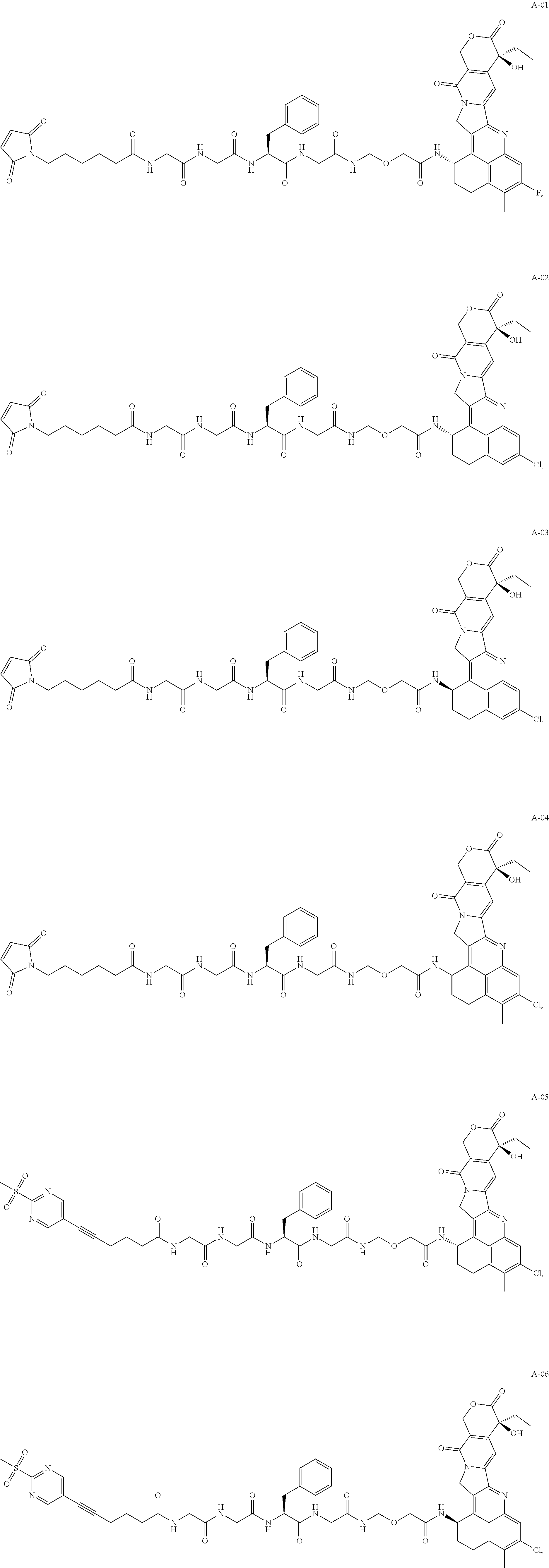

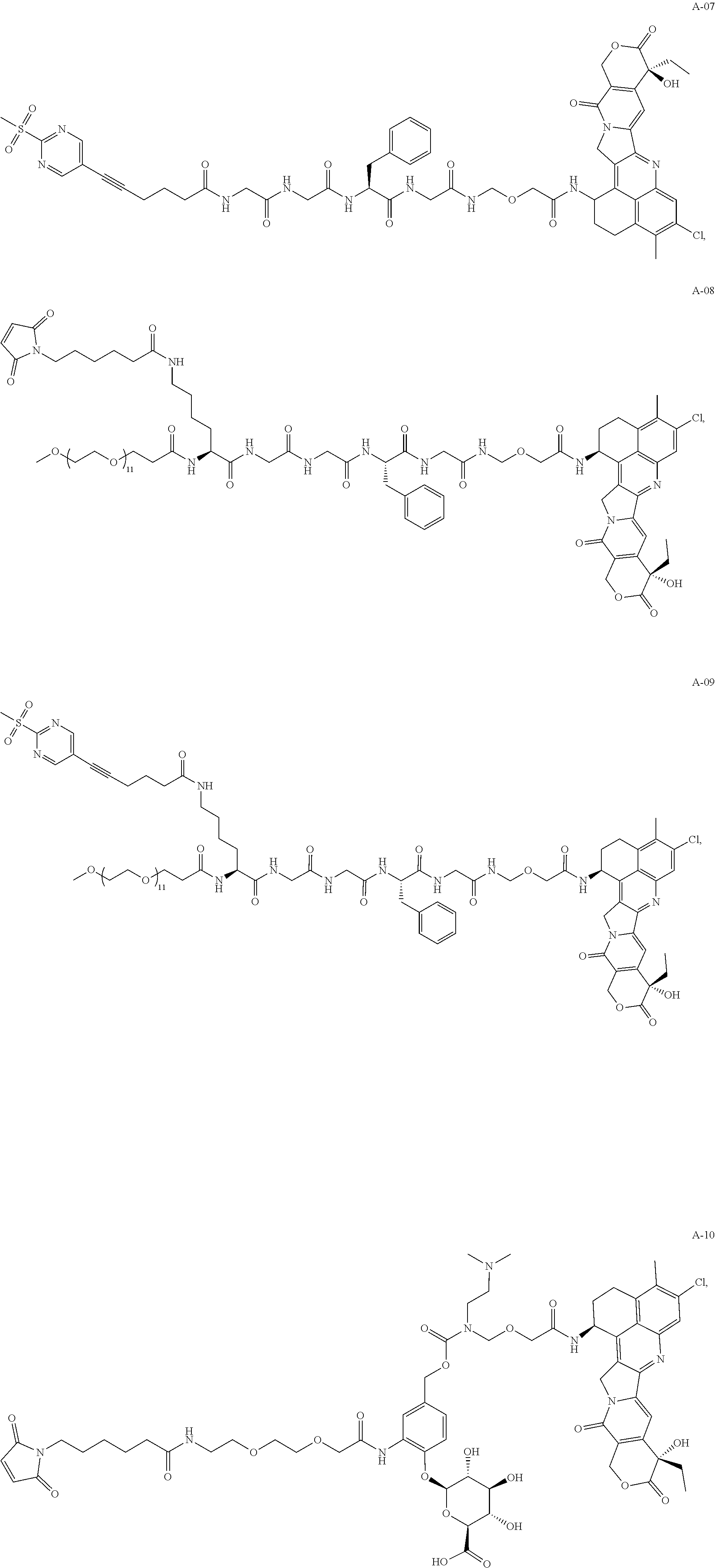

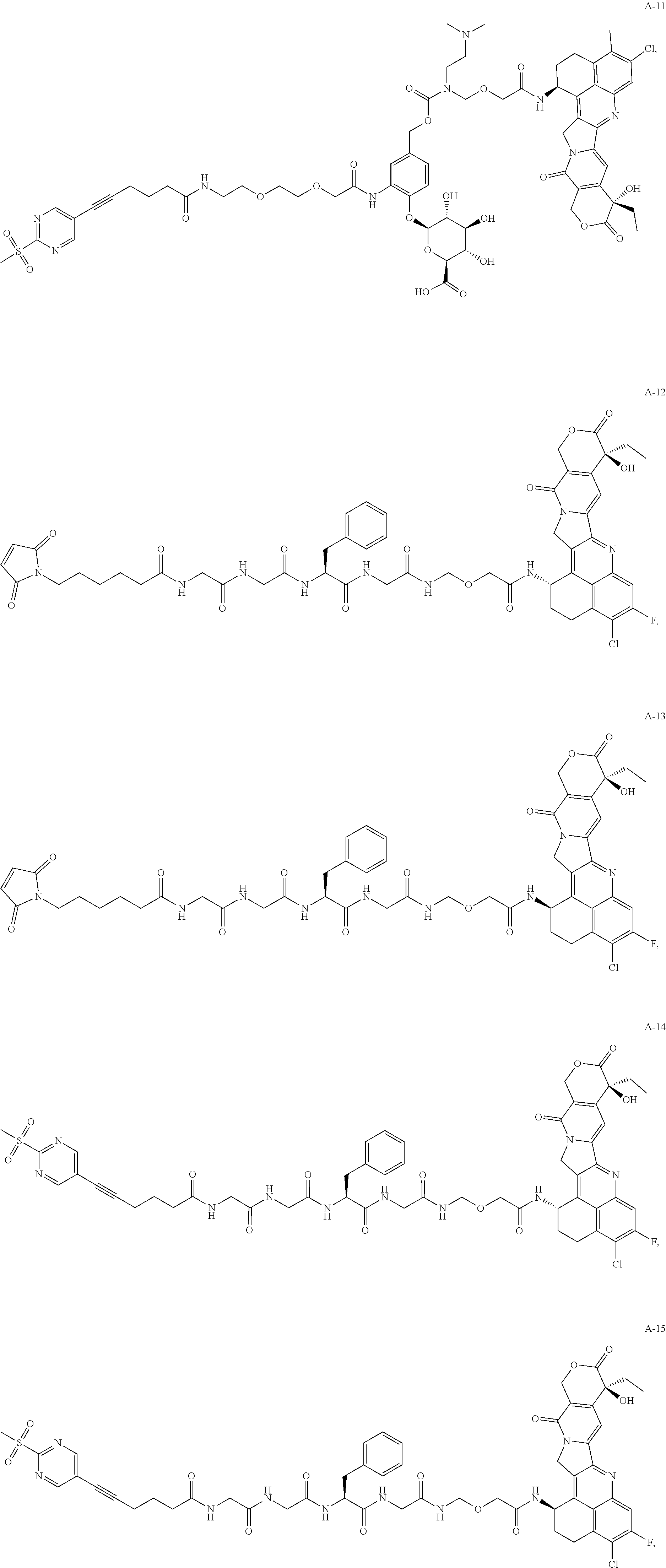

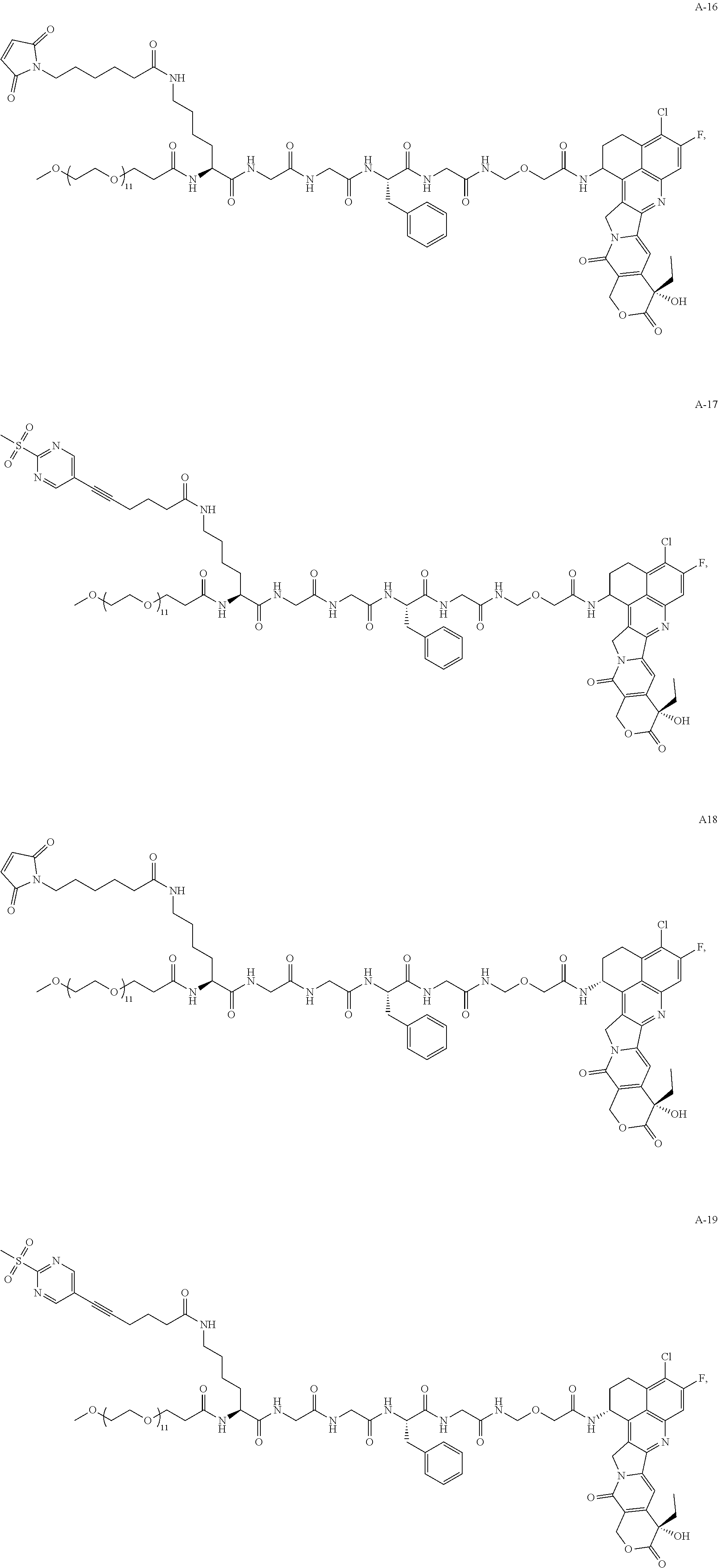

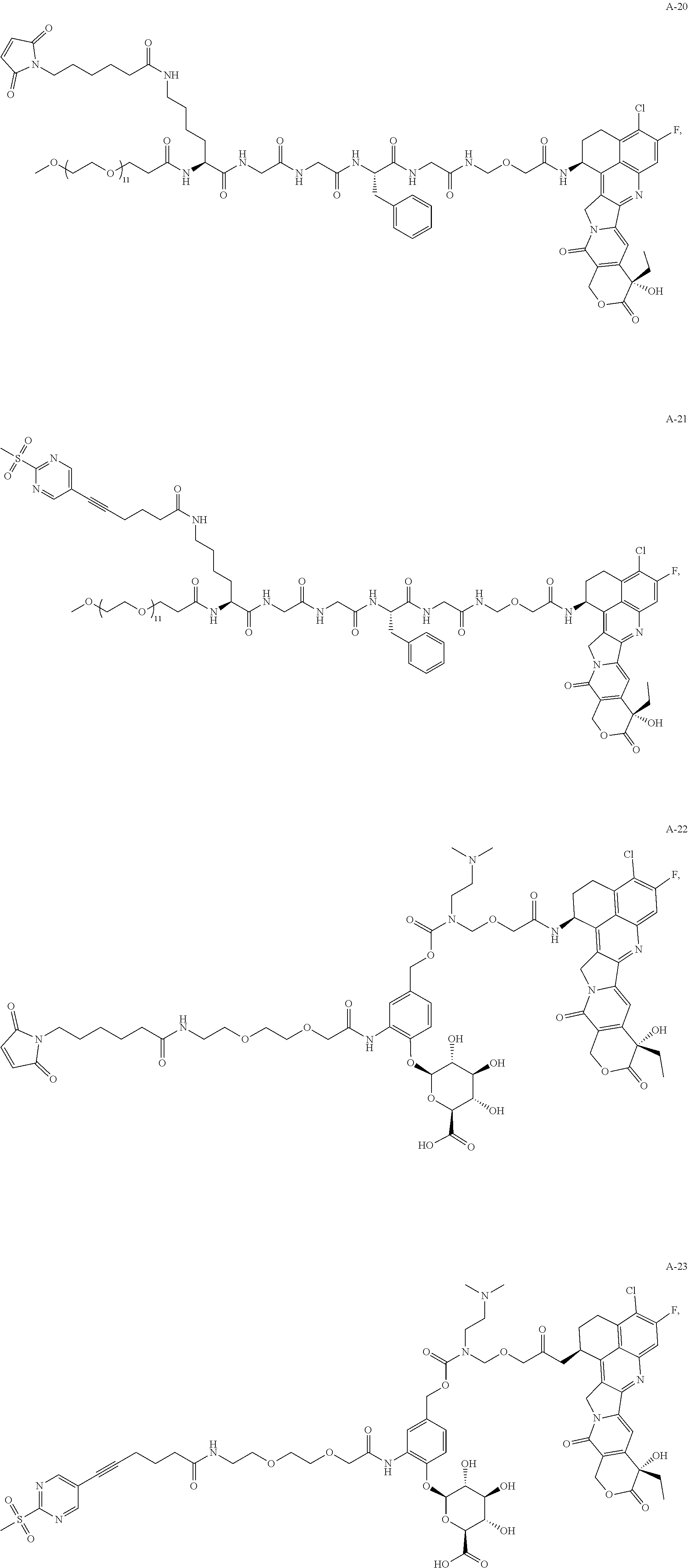

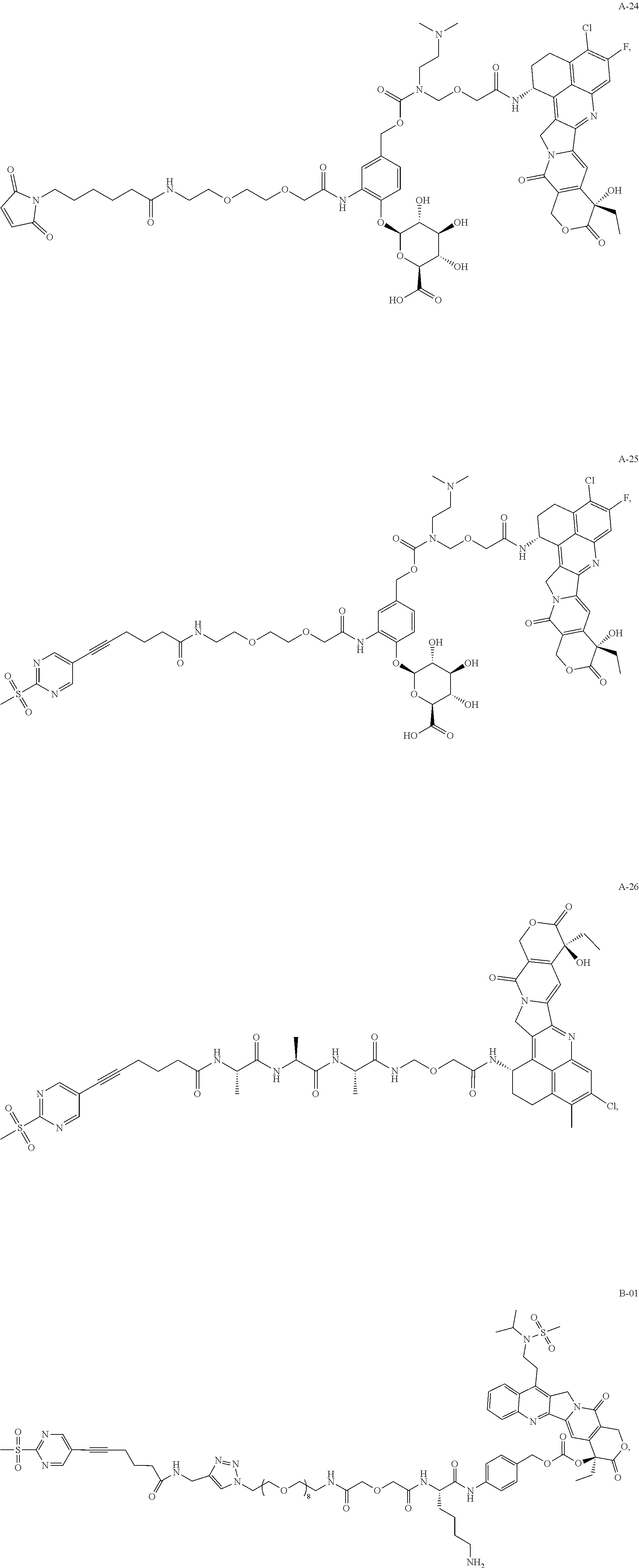

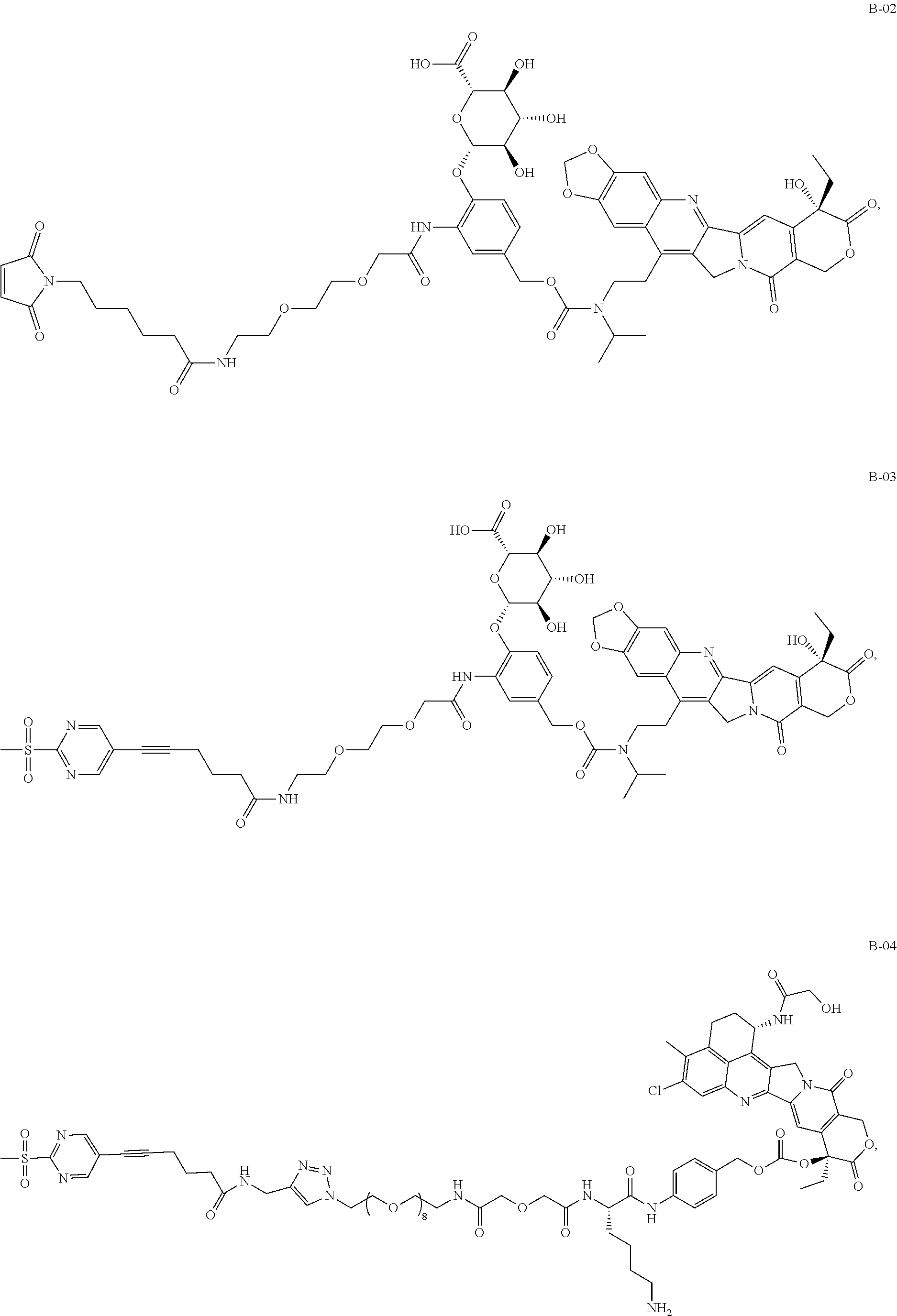

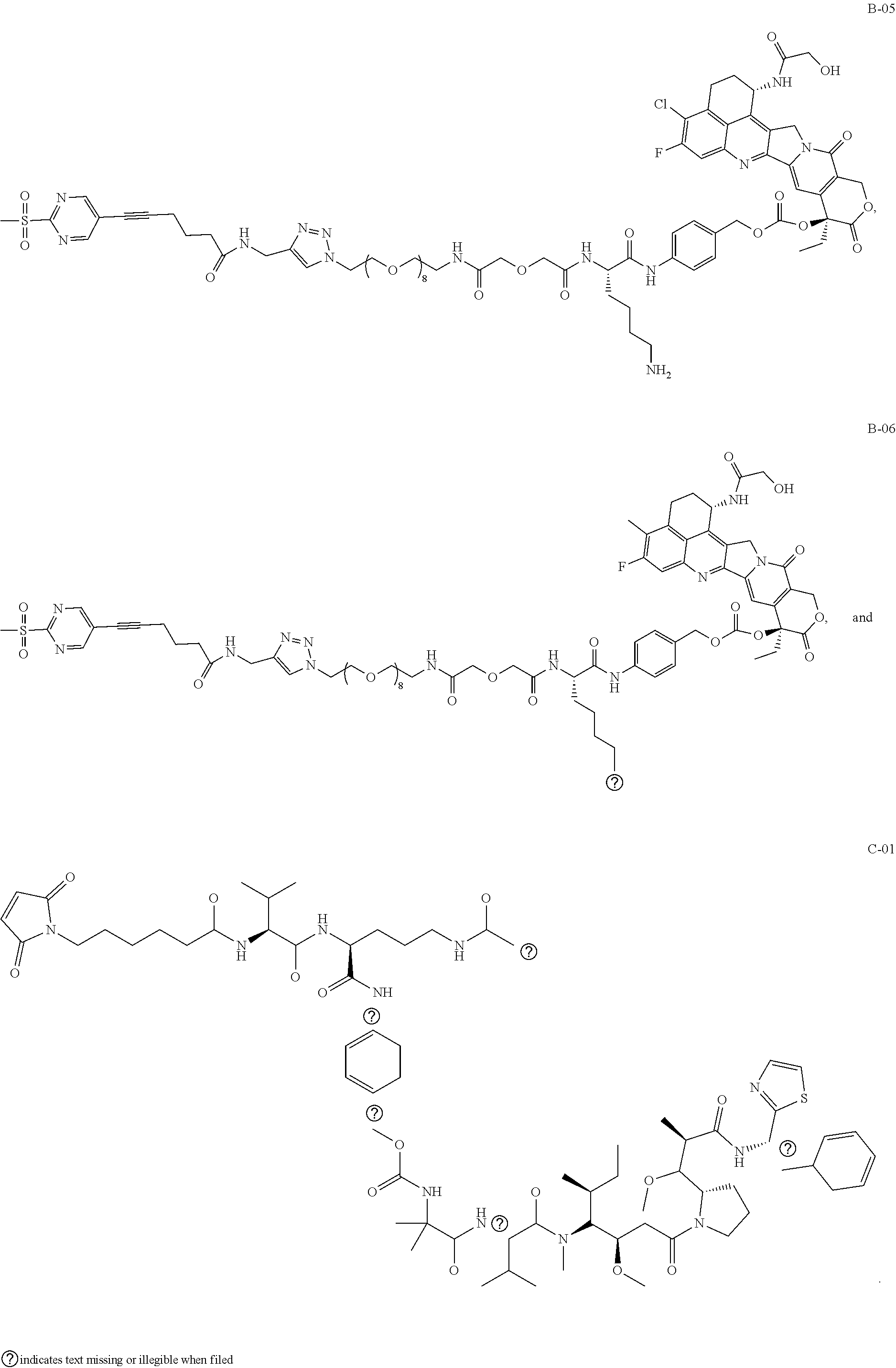

In some embodiments, the “drug-linker” in free form is selected from A-01 to A-25, B-01 to B-06, and C-01 shown as follows:

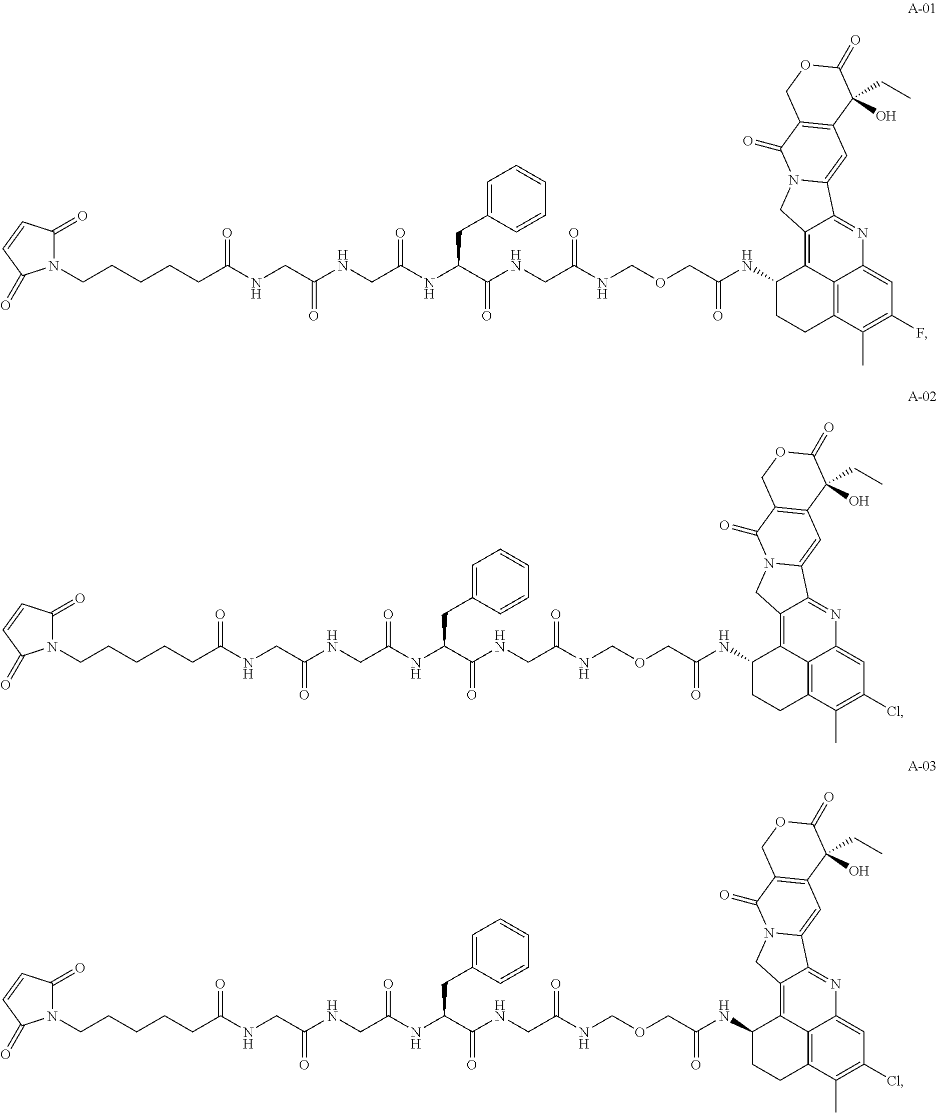

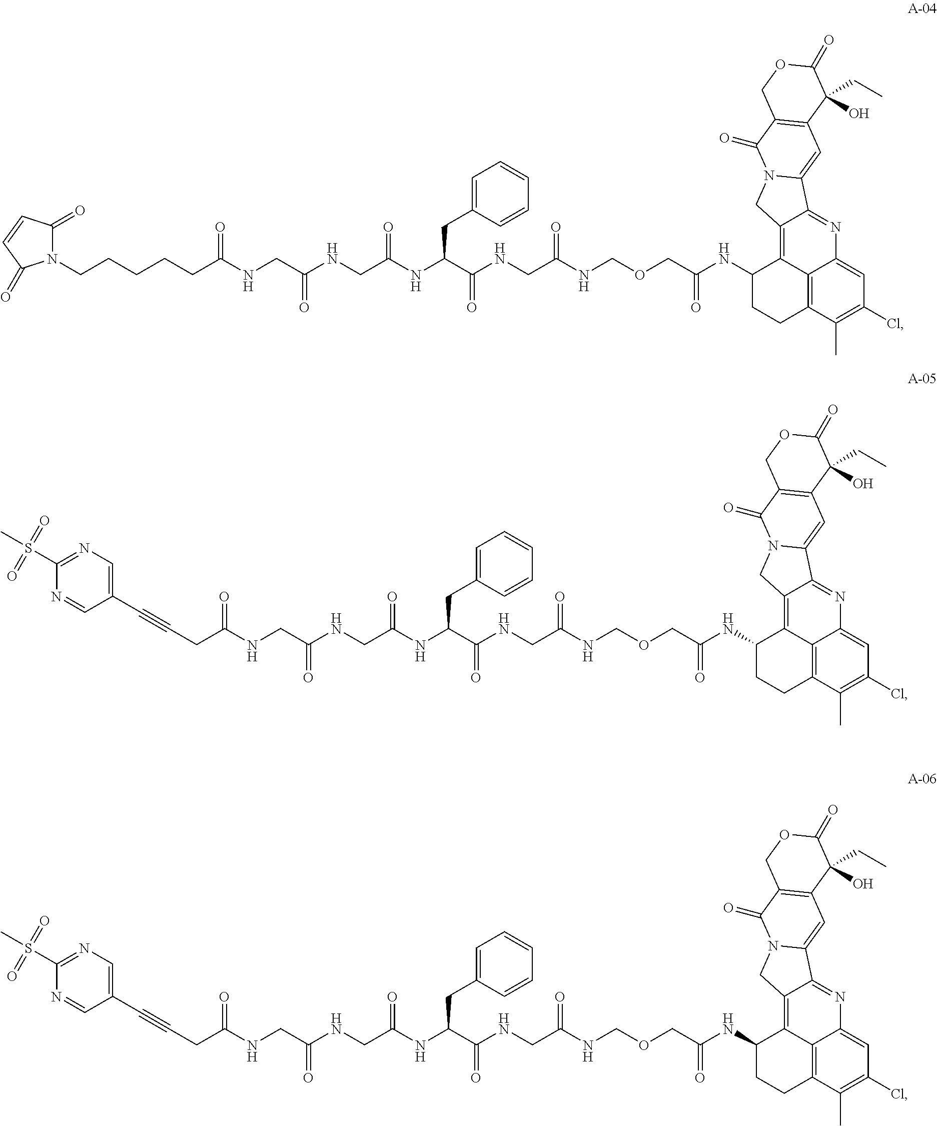

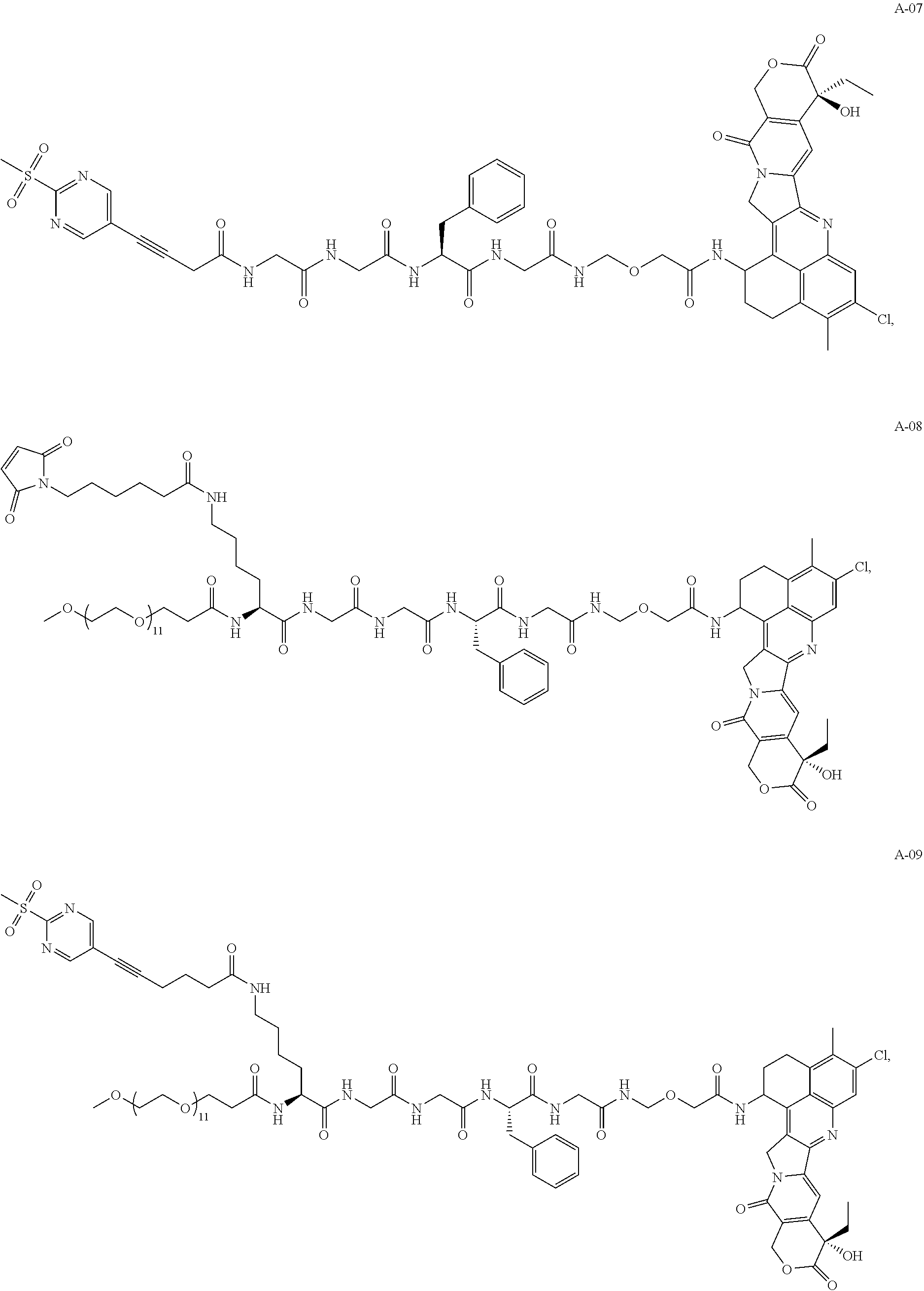

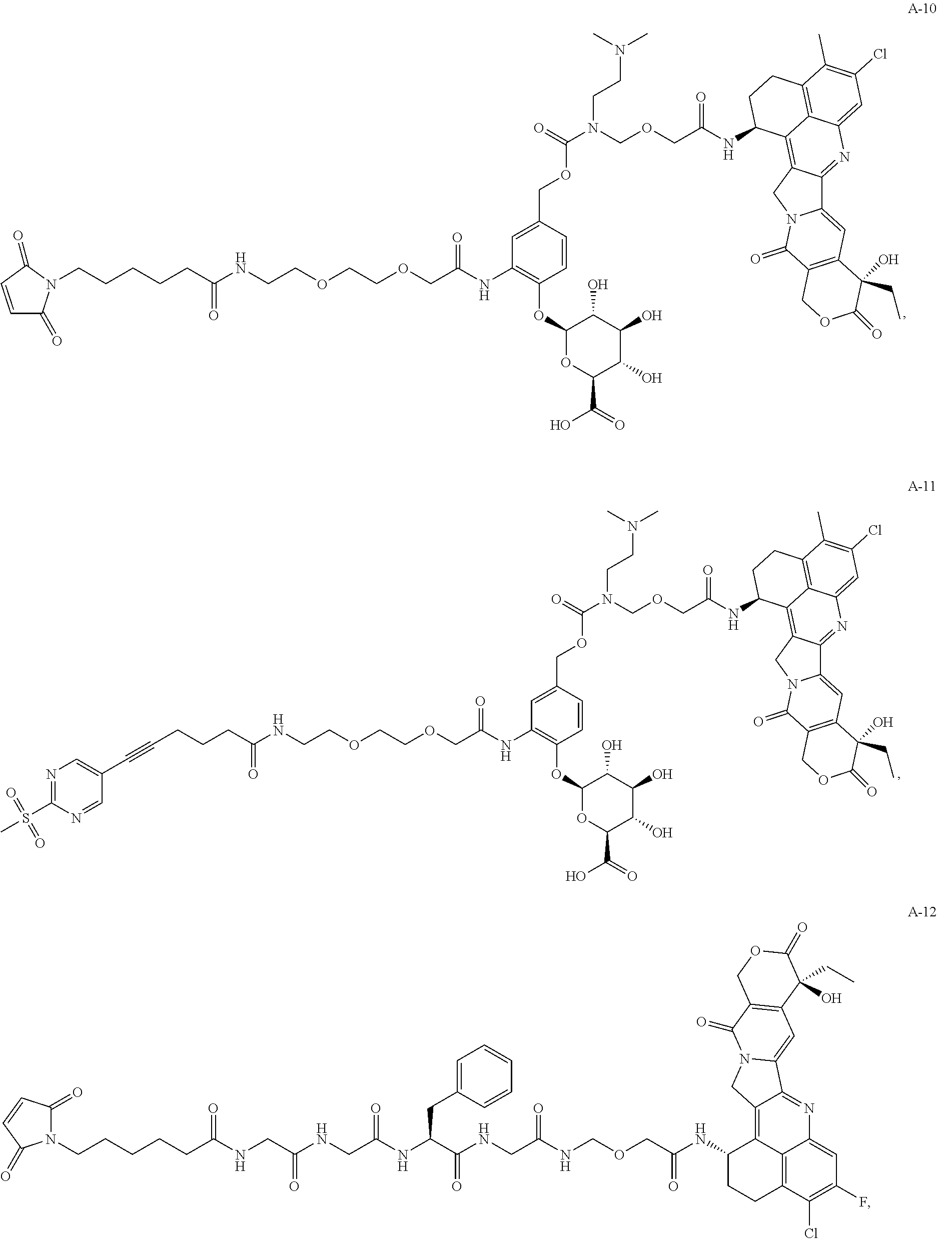

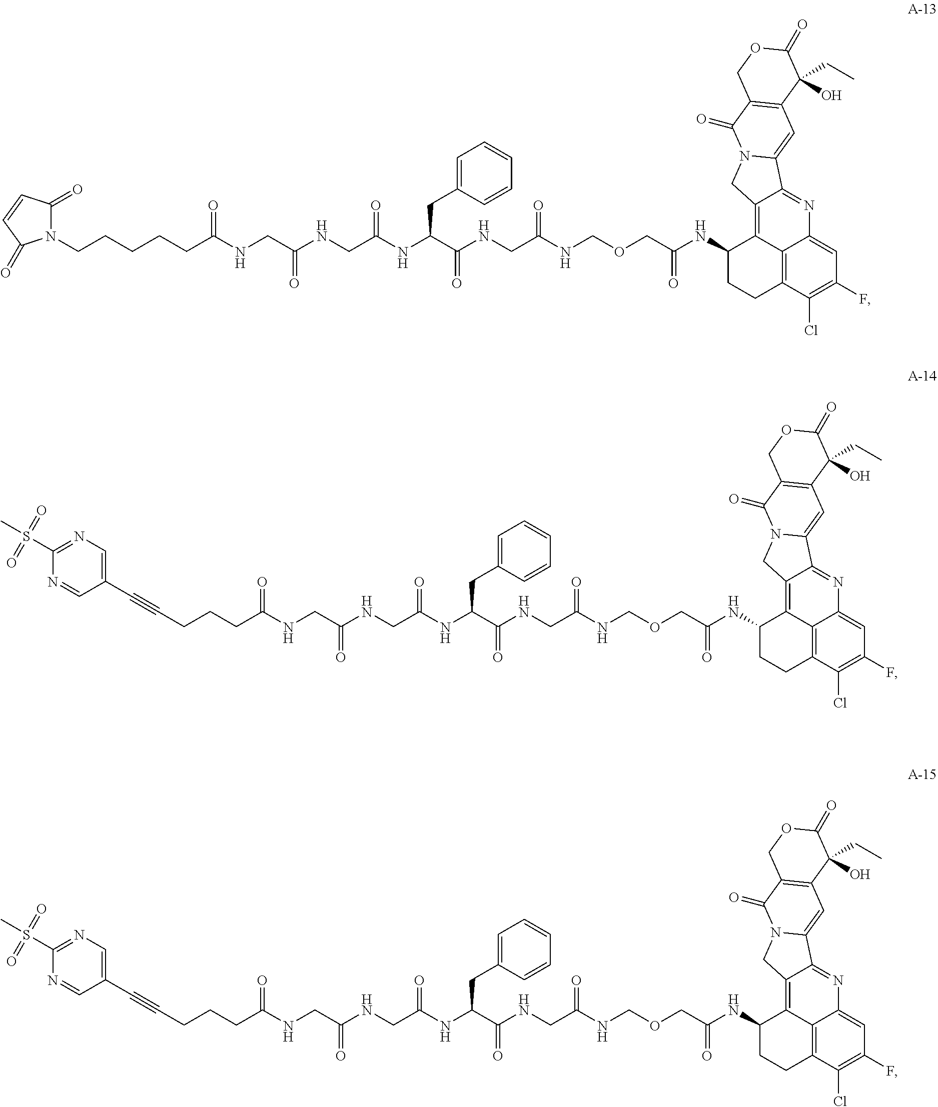

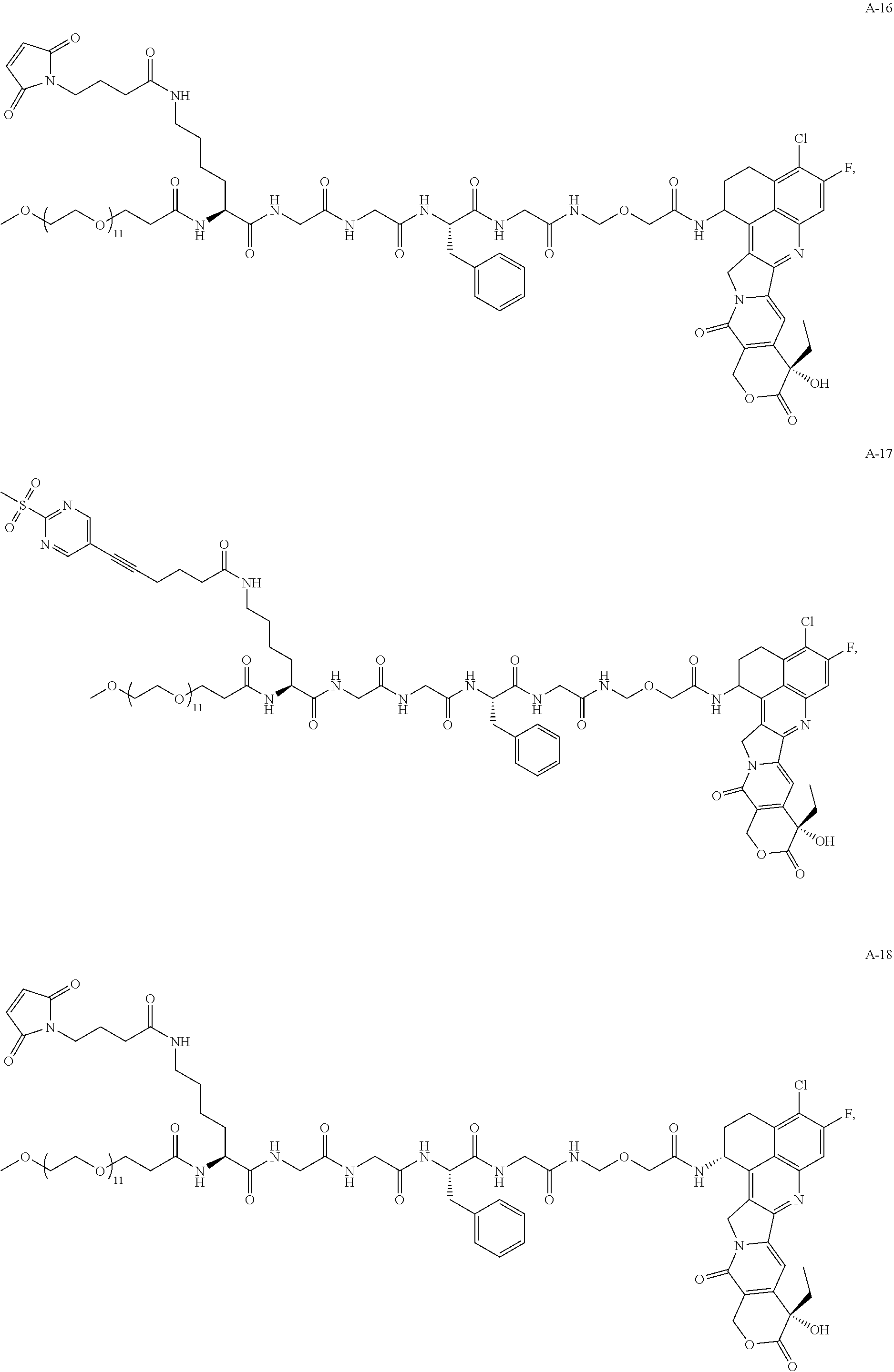

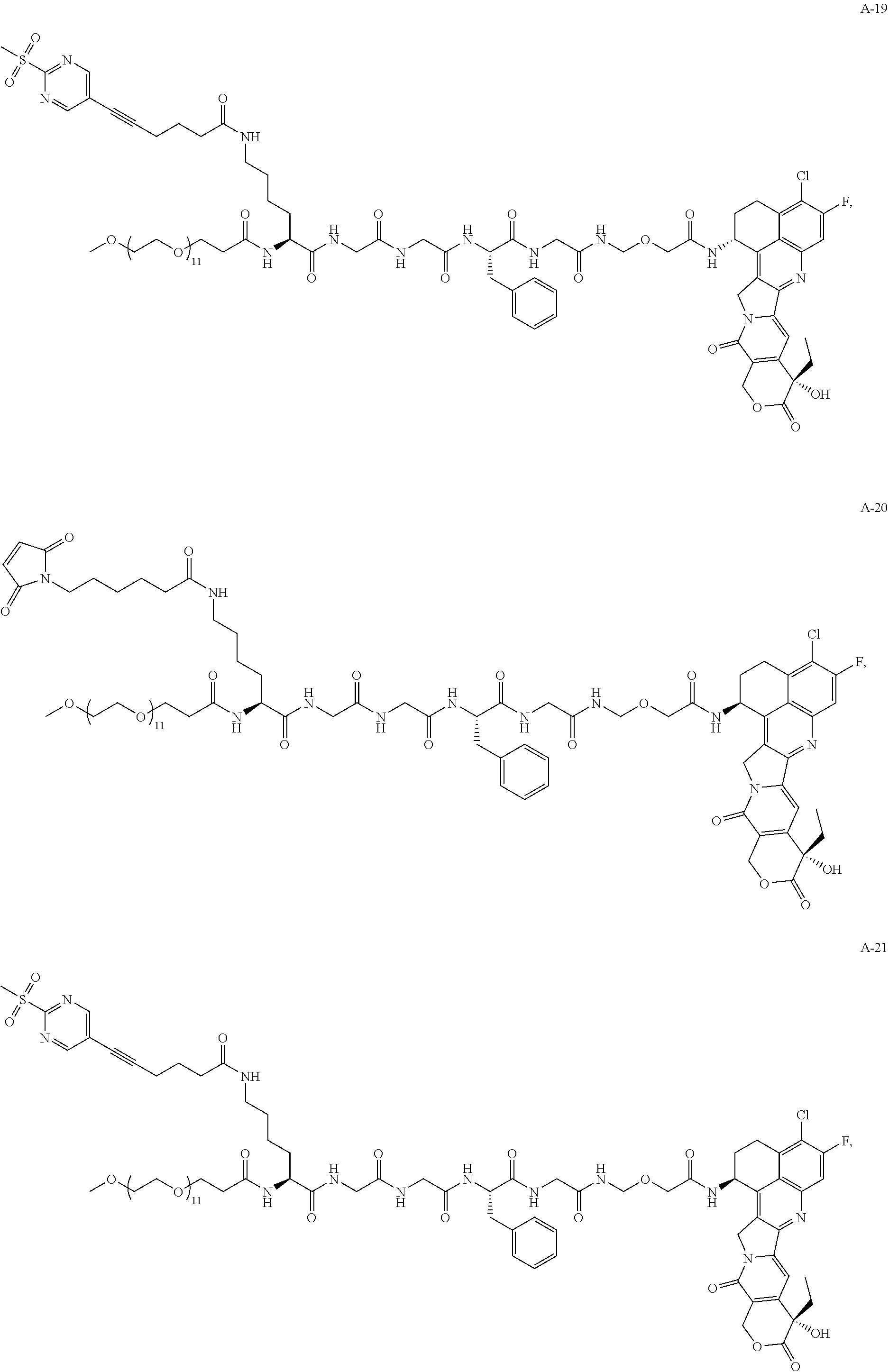

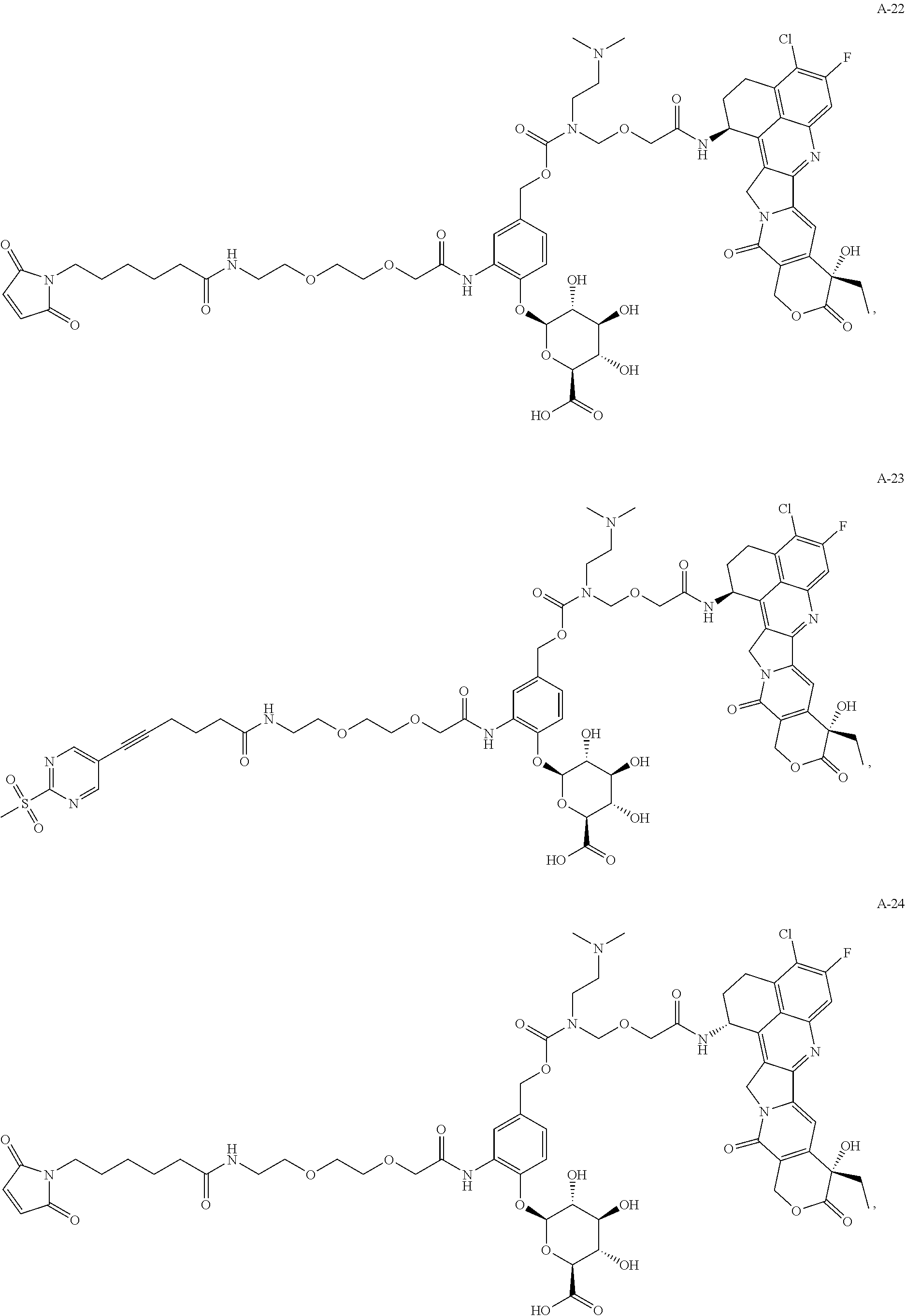

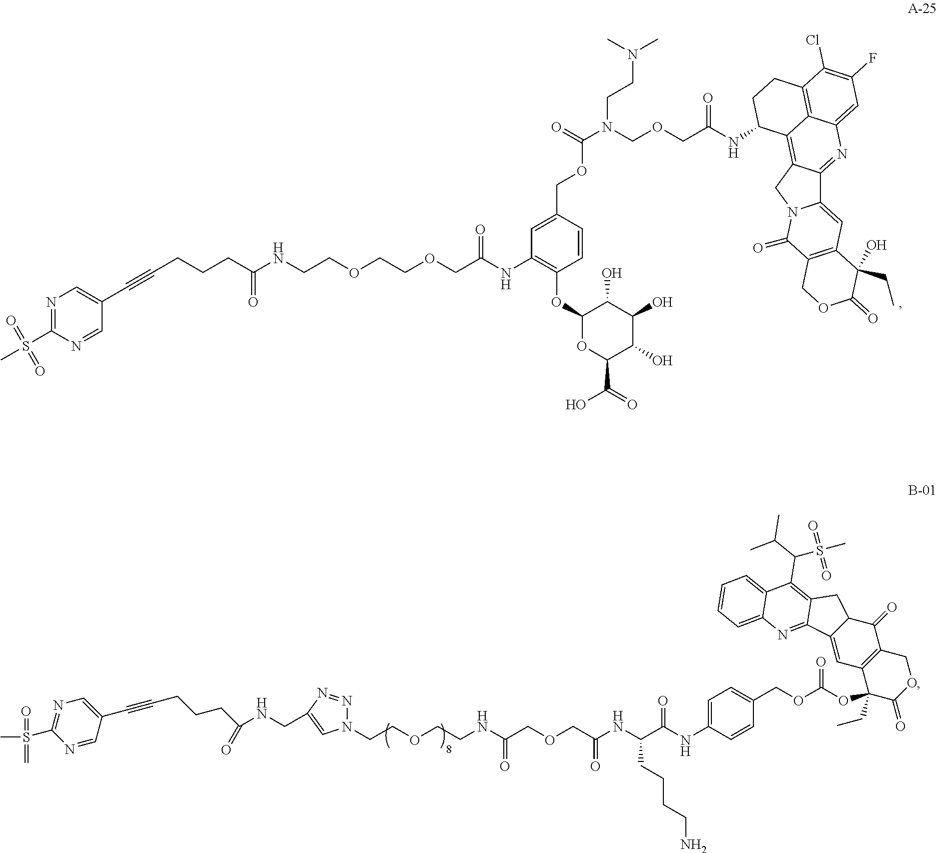

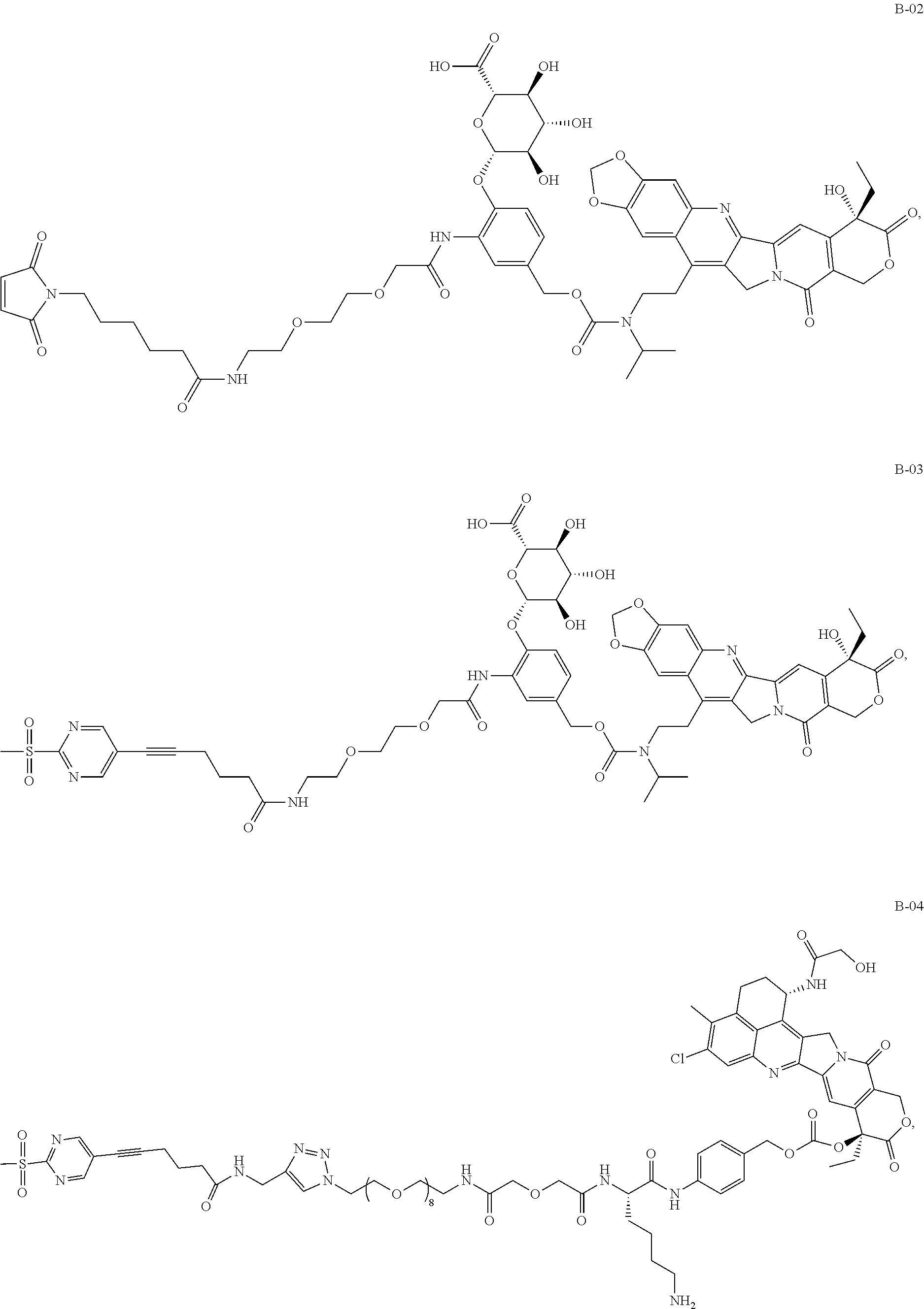

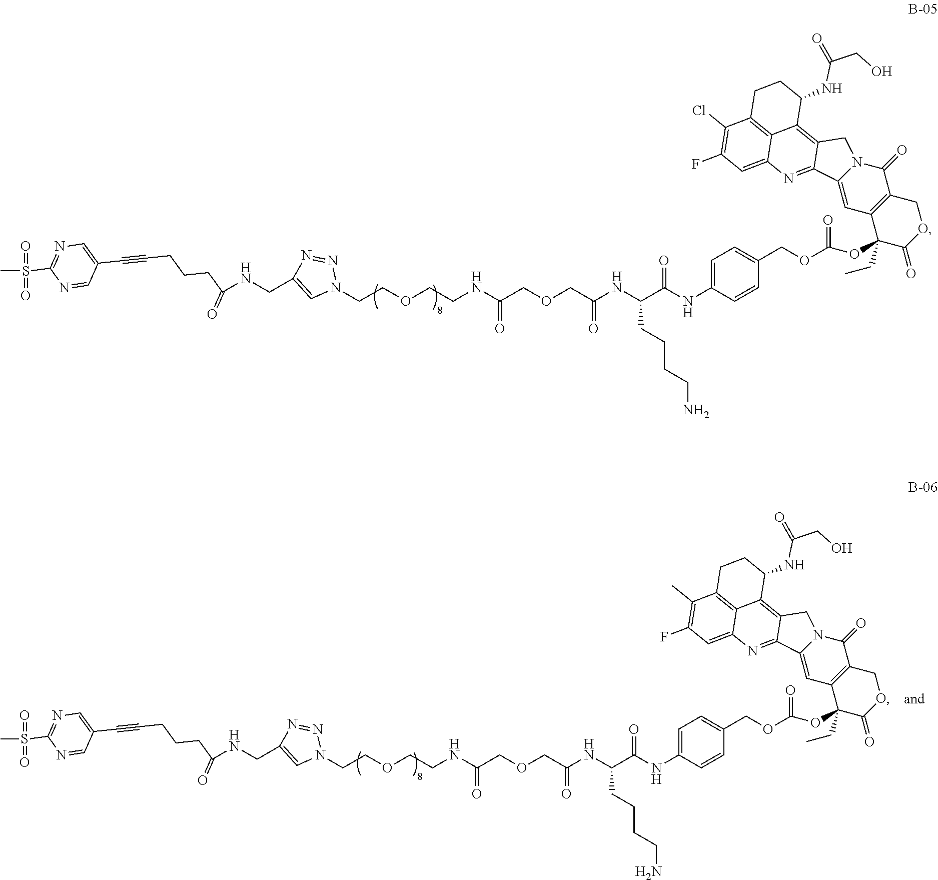

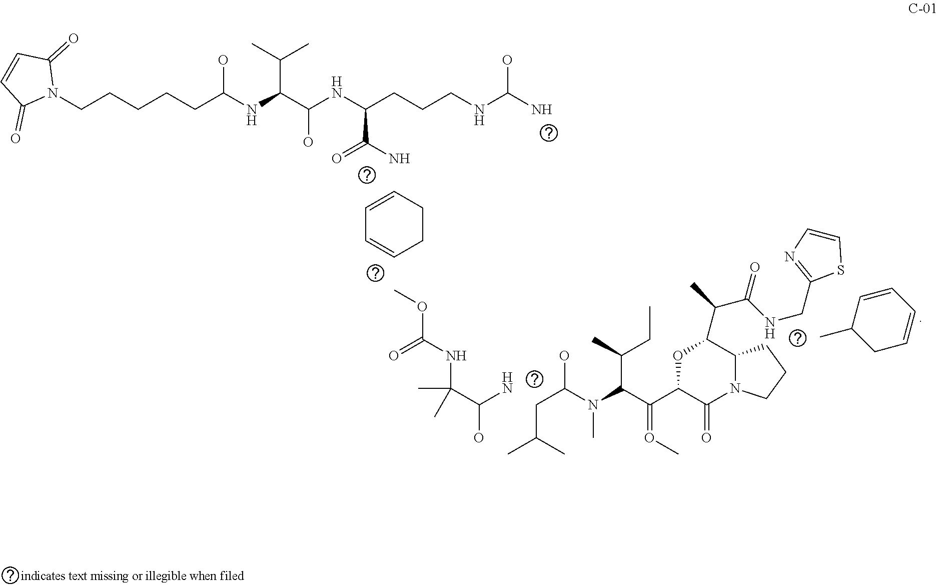

### Pharmaceutical Compositions

In another aspect, the present application provides a pharmaceutical composition, comprising the antibody-drug conjugate according to any one of the foregoing, optionally the drug-linker according to any one of the foregoing, and one or more pharmaceutic adjuvant(s).

The antibody-drug conjugate described herein is typically formulated in a unit injectable form together with a pharmaceutically acceptable parenteral vehicle for parenteral use, for example bolus injection, intravenous injection, intratumoral injection and the like. Optionally, the antibody-drug conjugate with desired purity is mixed with a pharmaceutically acceptable diluent, carrier, excipient or stabilizer in the form of lyophilizate or solution (Remington's Pharmaceutical Sciences (1980) 16^{th} edition, Osol, A. Ed.). The antibody-drug conjugate, or the pharmaceutical composition comprising the antibody-drug conjugate described herein may be administered by any route appropriate for the individual to be treated.

### Use

The antibody-drug conjugate or the pharmaceutical composition thereof described herein may be used for treating various diseases or disorders, for example cancers with high expression of PTK7, including solid tumors or hematological malignancies, for example lung cancer, breast cancer, epidermal cancer (for example, squamous cell carcinoma of skin, and oral squamous cell carcinoma), ovarian cancer, esophageal cancer (for example, esophageal squamous cell carcinoma) and the like.

Therefore, the present application provides use of the antibody-drug conjugate, drug-linker, or pharmaceutical composition comprising the same in preparation of a drug for treating cancer with high expression of PTK7.

Meanwhile, the present application also provides a method for treating cancers with high expression of PTK7, which includes steps of: administering an effective amount of the antibody-drug conjugate, drug linker, or pharmaceutical composition comprising the same according to any one of the foregoing to a subject in need. In particular embodiments, the cancer comprises solid tumors or hematological malignancies. In a further embodiment, the cancer is lung cancer, breast cancer, epidermal cancer (for example, squamous cell carcinoma of skin, and oral squamous cell carcinoma), ovarian cancer, esophageal cancer (for example, esophageal squamous cell carcinoma) and the like.

### BRIEF DESCRIPTION OF THE DRAWINGS

FIG. 1A: Detection of efficacy of different antibody-drug conjugates in an NCI-H358 model.

FIG. 1B: Body weight detection of different antibody-drug conjugates in an NCI-H358 model.

FIG. 2A: Efficacy detection of different antibody-drug conjugates in an A431 model.

FIG. 2B: Body weight detection of different antibody-drug conjugates in an A431 model.

FIG. 3A: Detection of binding of an anti-human PTK7 antibody-drug conjugate to OVCAR3 cells.

FIG. 3B: Detection of binding of an anti-human PTK7 antibody-drug conjugate to NCI-H358 cells.

FIG. 3C: Detection of binding of an anti-human PTK7 antibody-drug conjugate to HCC1806 cells.

FIG. 3D: Detection of binding of an anti-human PTK7 antibody-drug conjugate to A431 cells.

FIG. 3E: Detection of binding of anti-human PTK7 antibody-drug conjugates to NCI-H520 cells.

FIG. 3F: detection of binding of anti-human PTK7 antibody-drug conjugates to DU4475 cells.

FIG. 4A: pHrodo detection of endocytosis of antibody-drug conjugates in NCI-H358.

FIG. 4B: pHrodo detection of endocytosis of antibody-drug conjugates in HCC1806.

FIG. 4C: pHrodo detection of endocytosis of antibody-drug conjugates in OVCAR3.

FIG. 4D: pHrodo detection of endocytosis of antibody-drug conjugates in NCI-H520.

FIG. 4E: pHrodo detection of endocytosis of antibody-drug conjugates in DU4475.

FIG. 5A: Detection of anti-human PTK7-conjugated drug in killing of FADU cells.

FIG. 5B: Detection of anti-human PTK7-conjugated drug in killing of HCC1806 cells.

FIG. 5C: Detection of anti-human PTK7-conjugated drug in killing of DU4475 cells.

FIG. 6A: Detection of efficacy of an antibody-drug conjugate in an HCC1806 model.

FIG. 6B: Detection of body weight detection of an antibody-drug conjugate in an HCC1806 model.

FIG. 7A: Detection of efficacy of an antibody-drug conjugate in an OVCAR3 model.

FIG. 7B: Detection of body weight of an antibody-drug conjugate in an OVCAR3 model.

FIG. 8A: Detection of efficacy of an antibody-drug conjugate in an NCI-H146 model.

FIG. 8B: Detection of body weight of an antibody-drug conjugate in an NCI-H146 model.

### DETAILED DESCRIPTION

### Definitions

Unless otherwise defined hereinafter, all technical and scientific terms as used herein have the same meaning as commonly understood by those skilled in the art. References to techniques used herein are intended to refer to techniques commonly understood in the art, including those variations of the techniques or replacements of equivalent techniques that are obvious to those skilled in the art. Moreover, the laboratory operation steps such as genomics, nucleic acid chemistry, and molecular biology used herein are all routine steps widely used in the respective fields. While the following terms are believed to be well understood by those skilled in the art, the following definitions are set forth to better explain the present invention.

The term “antibody” generally refers to immunoglobulin molecules that are composed of two pairs of polypeptide chains (each pair having one light chain (LC) and one heavy chain (HC)). The light chain of the antibody can be classified as kappa (κ) and lambda (λ) light chains. The heavy chain can be classified as μ, δ, γ, α or ε, and the isotypes of the antibody are defined as IgM, IgD, IgG, IgA, and IgE, respectively. In the light chain and heavy chain, the variable regions and the constant regions are linked by a “J” region having about 12 or more amino acids, and the heavy chain further comprises a “D” region having about 3 or more amino acids. Each heavy chain is comprised of the heavy chain variable region (VH) and the heavy chain constant region (CH). The heavy chain constant region is composed of 3 domains (CH1, CH2, and CH3). Each light chain comprises the light chain variable region (VL) and the light chain constant region (CL). The light chain constant region is composed of one domain CL. The constant domains do not directly participate in the binding between the antibody and the antigen, but show a variety of effector functions, such as mediating the binding of immunoglobulins to host tissues or factors, including various cells of the immune system (like effector cells) and a first component (C1q) of a classical complement system. The VH and VL regions may also be subdivided into regions with high variability (referred to as complementarity determining regions (CDRs)), with more conservative regions interspersed therebetween, referred to as framework regions (FRs). Each VH and each VL are composed of 3 CDRs and 4 FRs arranged in the following order: FR1, CDR1, FR2, CDR2, FR3, CDR3 and FR4 from the amino terminus to the carboxy terminus. The variable regions (VH and VL) of each heavy chain/light chain pair form antigen-binding sites, respectively. The allocation of amino acids in each region or domain is according to various numbering system known in the art.

The term “complementarity determining region” or “CDR” refers to amino acid residues responsible for antigen-binding in the variable region of the antibody. The variable regions of the heavy chain and the light chain each comprises three CDRs, named as CDR1, CDR2 and CDR3. The exact boundaries of these CDR can be defined according to various numbering systems known in the art, e.g., as defined according to a Kabat numbering system (Kabat et al., Sequences of Proteins of Immunological Interest, 5th Ed. Public Health Service, National Institutes of Health, Bethesda, Md., 1991), a Chothia numbering system (Chothia & Lesk (1987) J. Mol. Biol. 196:901-917; Chothia et al. (1989) Nature 342:878-883), an IMGT numbering system (Lefranc et al., Dev. Comparat. Immunol. 27:55-77, 2003), or an AbM numbering system (Martin A C R, Cheetham J C, Rees A R (1989) Modelling antibody hypervariable loops: A combined algorithm. Proc Natl Acad Sci USA 86:9268-9272). For a given antibody, those skilled in the art will easily identify the CDRs defined according to each numbering system. Moreover, the correspondence between different numbering systems is well-known to those skilled in the art (like, referring to Lefranc et al., Dev. Comparat. Immunol. 27:55-77, 2003).

In the present invention, the CDR in the antibody or antigen-binding fragment can be determined according to various numbering system known in the art, e.g., as determined according to the Kabat, Chothia, IMGT or AbM numbering system. In certain embodiments, the CDR contained in the antibody or antigen-binding fragment is defined according to the Chothia numbering system.

The following general rules disclosed in www.bioinf.org.uk: Prof. Andrew C. R. Martin's Group and reproduced below may be used to define the CDRs in an antibody sequence that includes those amino acids that specifically interact with the amino acids comprising the epitope in the antigen to which the antibody binds. There are rare examples where these generally constant features do not occur; however, the Cys residues are the most conserved feature.

| Loop | Kabat | AbM | Chothia^{l} | Contact² | IMGT |
|---|---|---|---|---|---|
| L1 | L24—L34 | L24—L34 | L24—L34 | L30—L36 | L27—L32 |
| L2 | L50—L56 | L50—L56 | L50—L56 | L46—L55 | L50—L52 |
| L3 | L89—L97 | L89—L97 | L89—L97 | L89—L96 | L89—L97 |
| H1 | H31—H35B | H26—H35B | H26—H32 . . . 34 | H30—H35B | H26—H35B |
| | (Kabat Numbering)³ | | | | |
| H1 | H31—H35 | H26—H35 | H26—H32 | H30—H35 | H26—H33 |
| | (Chothia Numbering) | | | | |
| H2 | H50—H65 | H50—H58 | H52—H56 | H47—H58 | H51—H56 |
| H3 | H95—H102 | H95—H102 | H95—H102 | H93—H101 | H93—H102 |

¹Some of these numbering schemes (particularly for Chothia loops) vary depending on the individual publication examined.
²Any of the numbering schemes can be used for these CDR definitions, except the Contact numbering scheme uses the Chothia or Martin (Enhanced Chothia) definition.
³The end of the Chothia CDR-H1 loop when numbered using the Kabat numbering convention varies between H32 and H34 depending on the length of the loop. (This is because the Kabat numbering scheme places the insertions at H35A and H35B.)
If neither H35A nor H35B is present, the loop ends at H32
If only H35A is present, the loop ends at H33
If both H35A and H35B are present, the loop ends at H34

The entire amino acid sequence of the V_{H} is commonly numbered according to Kabat while the three CDRs within the variable region may be defined according to any one of the aforementioned numbering schemes. In particular embodiments, the numbering of the amino acid positions in the V_{H} may be sequential beginning with amino acid position 1 and continuing sequentially to the end of the sequence or according to Kabat. Unless specified otherwise, the amino acid positions in the V_{H} and V_{L} herein are defined according to sequential numbering.

The numbering of the amino acid positions in the heavy chain constant domain may be sequential beginning with amino acid position 1 and continuing sequentially to the end of the sequence or according to Eu numbering. The IgG1 heavy chain constant domain amino acid sequence has 330 amino acids sequentially numbered 1 to 330. The corresponding sequence numbered according to Eu begins with position number 118 and ends with position number 447. Unless specified otherwise, the amino acid positions in the heavy and light chains herein are defined according to sequential numbering.

The term “framework region” or “FR” residue refers to those amino acid residues in the variable region of the antibody other than the CDR residues as defined above.

The term “antigen-binding fragment” of antibody refers to polypeptide of a fragment of the antibody, for example polypeptide of a fragment of a full-length antibody, which retains the capability of specifically binding to the same antigen to which the full-length antibody is bound, and/or competes for specific binding to the antigen with the full-length antibody, which is also referred to as the “antigen-binding fragment”. Generally, refer to Fundamental Immunology, Ch. 7 (Paul, W., ed., 2nd Edition, Raven Press, N.Y. (1989), which is incorporated herein by reference for all purposes in its entirety. The antigen-binding fragment of the antibody may be produced by a recombinant DNA technology or by enzymatic or chemical cleavage of the complete antibody. Non-limiting examples of the antigen-binding fragment include a Fab fragment, a Fab′ fragment, a F(ab)′₂ fragment, a F(ab)′₃ fragment, Fd, Fv, scFv, di-scFv, (scFv)₂, disulfide bond stabilized Fv protein (“dsFv”), a single-domain antibody (sdAb, nanobody) and similar polypeptides, which comprise at least a part of the antibody sufficient to enable antigen-specific binding ability to the polypeptide. Engineered variants of the antibodies are summarized in Holliger et al., 2005; Nat Biotechnol, 23:1126-1136.

The term “Fd” means an antibody fragment composed of VH and CH1 domains; the term “dAb fragment” means an antibody fragment composed of VH domains (Ward et al., Nature 341:544 546 (1989)); the term “Fab fragment” means an antibody fragment composed of VL, VH, CL, and CH1 domains; the term “F(ab′)₂ fragment” means an antibody fragment comprising two Fab fragments linked by a disulfide bridge on the hinge region; the term “Fab′ fragment” means a fragment obtained after reducing the disulfide bond linking the two heavy chain fragments in the F(ab′)₂ fragment, and the obtained fragment is composed of a complete Fd fragment (composed of VH and CH1 domains) of the light chain and the heavy chain.

The term “Fv” means an antibody fragment composed of VL and VH domains of a single arm of the antibody. The Fv fragment is generally considered to be a minimal antibody fragment that is capable of forming a complete antigen-binding site. It is generally considered that six CDRs enable the antigen-binding specificity to the antibody. However, one variable region (e.g., an Fd fragment, which contains only three CDRs specific to the antigen) also can be used for recognizing and binding to the antigen, although its affinity may be lower than the complete binding site.

The term “Fc” means an antibody fragment formed by binding second and third constant regions of the first heavy chain of the antibody to second and third constant regions of the second heavy chain through disulfide bonds. The Fc fragment of the antibody has a variety of different functions, but does not participate in antigen-binding.

The term “scFv” refers to a single polypeptide chain comprising VL and VH domains, where the VL and VH are linked by the linker (see, for example, Bird et al., Science 242:423-426 (1988); Huston et al., Proc. Natl. Acad. Sci. USA 85:5879-5883 (1988); and Pluckthun, The Pharmacology of Monoclonal Antibodies, Vol. 113, edited by Roseburg and Moore, Springer-Verlag, New York, Pages 269-315 (1994)). Such scFv molecules may have general structure: NH₂—VL-linker-VH—COOH or NH₂—VH-linker-VL-COOH. Suitable linker in the prior art comprises repeated GGGGS (SEQ ID NO: 55) amino acid sequences or variants thereof. For example, a linker having the amino acid sequence (GGGGS) 4 (SEQ ID NO: 56) can be used, and the variants thereof can also be used (Holliger, et al. (1993), Proc. Natl. Acad. Sci. USA 90:6444-6448). Other linkers applicable to the present invention are described by Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol. In some cases, there may be a disulfide bond between VL and VH of scFv. In certain embodiments, the VH and VL domains may be oppositely positioned to each other in any suitable arrangement. For example, scFv comprising NH₂—VH—VH—COOH and NH₂—VL-VL-COOH.

The term “single-domain antibody (sdAb)” has the meaning generally understood by those skilled in the art, which refers to an antibody fragment composed of a single monomer variable antibody domain (like a single heavy chain variable region), and such antibody fragment retains the capability of specifically binding the same antigen to which the full-length antibody binds (Holt, L. et al., Trends in Biotechnology, 21 (11): 484-490, 2003). The single-domain antibody is also called nanobody.

Each of the above antibody fragments retains the capability of specifically binding to the same antigen to which the full-length antibody binds, and/or competes with the full-length antibody for specific binding to the antigen.

Herein, unless otherwise specified, when referring to the term “antibody”, it includes not only the complete antibody, but also the antigen-binding fragment of the antibody.

The antigen-binding fragment of the antibody (like the antibody fragment above) can be obtained from a given antibody (like the antibody provided by the present invention) using conventional techniques known to those skilled in the art (like recombinant DNA techniques or enzymatic or chemical cleavage methods), and the antigen-binding fragment of the antibody is screened for specificity in the same manner as for the complete antibody.

The term “murine antibody” refers to an antibody obtained by the following methods: fusing B cells and myeloma cells of immunized mice, screening murine hybrid fusion cells that can both immortalize and secrete antibodies, and then performing screening, antibody preparation and antibody purification; or refers to the antibody secreted by plasma cells formed by the differentiation and proliferation of B cells after the antigen invades the mouse body.

The term “humanized antibody” refers to a genetically engineered non-human antibody of which the amino acid sequence has been modified to increase the sequence homology with a human antibody. Generally, all or part of the CDR regions of a humanized antibody are derived from a non-human antibody (donor antibody), and all or part of the non-CDR regions (for example, variable region FR and/or constant region) are derived from human Immunoglobulin (receptor antibody). The humanized antibody generally retains the expected properties of the donor antibody, including but not limited to, antigen specificity, affinity, reactivity, capability of enhancing immune cell activity, capability of enhancing immune response, and the like. The donor antibody can be mouse, rat, rabbit or non-human primate (like cynomolgus monkey) antibodies with desired properties (for example, antigen specificity, affinity, reactivity, capability of enhancing immune cell activity and/or capability of enhancing immune response).

As used herein, the term “identity” refers to the matching of sequences between two polypeptides or between two nucleic acids. When a position in two to-be-compared sequences is occupied by a same base or amino acid monomeric subunit (e.g., a position in each of two DNA molecules is occupied by adenine, or a position in each of the two polypeptides is occupied by lysine), the molecules are the same at that position. The “percentage identity” between the two sequences refers to a function obtained by the formula: the number of matched positions common to the two sequences/the number of to-be-compared positions×100. For example, if there are 6 matches in the 10 positions of the two sequences, the two sequences have 60% identity. For example, DNA sequences CTGACT and CAGGTT have 50% identity in total (3 matches in the total of 6 positions). Typically, comparison is performed when two sequences are aligned to achieve the maximum identity. Such alignment can be achieved by using, for example, a method proposed by Needleman et al. (1970) J. Mol. Biol. 48:443-453 and conveniently performed by a computer program, such as an Align program (DNAstar, Inc.). The percentage identity between two amino acid sequences can also be determined through E. Meyers and W. Miller (Comput. Appl Biosci., 4:11-17 (1988)) algorithms that are integrated into an ALIGN program (Version 2.0) by using a PAM120 weight residue table, a gap length penalty of 12, and a gap penalty of 4. In addition, the percentage identity between two amino acid sequences can be determined through Needleman and Wunsch (J Mol Biol. 48:444-453 (1970)) algorithms in a GAP program that is integrated into a GCG software package (available at www.gcg.com) by using a Blossum 62 matrix or PAM250 matrix and gap weight of 16, 14, 12, 10, 8, 6 or 4 and length weights of 1, 2, 3, 4, 5 or 6.

The term “conservative replacement” means amino acid replacement that does not adversely affect or change the intended properties of proteins/polypeptides comprising the amino acid sequence. For example, the conservative replacement can be introduced by standard techniques known in the art, for example site-directed mutagenesis and PCR-mediated mutagenesis. Conservative amino acid replacement includes replacement of amino acid residues with amino acid residues having similar side chains, e.g., replacement with residues that are physically or functionally similar to the corresponding amino acid residues (e.g., having similar size, shape, charge, chemical properties, including the capability of forming covalent or hydrogen bonds). Families of the amino acid residues having similar side chains have been defined in the art. These families include amino acids having basic side chains (for example, lysine, arginine, and histidine), acidic side chains (for example, aspartic acid, and glutamic acid), uncharged polar side chains (for example, glycine, asparagine, glutamine, serine, threonine, tyrosine, cysteine, and tryptophan), non-polar side chains (for example, alanine, valine, leucine, isoleucine, proline, phenylalanine, and methionine), β-branched side chains (for example, threonine, valine, and isoleucine), and aromatic side chains (for example, tyrosine, phenylalanine, tryptophan, and histidine). Thus, the corresponding amino acid residue is preferably replaced with another amino acid residue from the same side chain family. Methods for identifying amino acid conservative replacements are well-known in the art (see, for example, Brummell et al., Biochem. 32:1180-1187 (1993); Kobayashi et al. Protein Eng. 12 (10): 879-884 (1999); and Burks et al. Proc. Natl Acad. Set USA 94:412-417 (1997), which is incorporated herein by reference).

The compilation of the twenty conventional amino acids involved herein follows conventional usage. See, for example, Immunology-A Synthesis (2nd Edition, E. S. Golub and D. R. Gren, Eds., Sinauer Associates, Sunderland, Mass. (1991)), which is incorporated herein by reference. In the present invention, the amino acid is generally shown as a single letter or three letter abbreviation well known in the art. For example, alanine may be shown as A or Ala.

The term “pharmaceutically acceptable carrier and/or excipient” refers to a carrier and/or excipient that is pharmacologically and/or physiologically compatible with the subject and the active ingredient, It is well known in the art (see e.g. Remington's Pharmaceutical Sciences. Edited by Gennaro AR, 19th ed. Pennsylvania: Mack Publishing Company, 1995), and includes but is not limited to: pH adjusters, surfactants, adjuvants, ionic strength enhancers, diluents, agents to maintain osmotic pressure, agents to delay absorption, preservatives. For example, pH adjusting agents include, but are not limited to, phosphate buffers. Surfactants include but are not limited to cationic, anionic or nonionic surfactants such as Tween-80. Ionic strength enhancers include, but are not limited to, sodium chloride. Preservatives include, but are not limited to, various antibacterial and antifungal agents, such as parabens, chlorobutanol, phenol, sorbic acid, and the like. Agents to maintain osmotic pressure include, but are not limited to, sugars, NaCl, and the like. Agents that delay absorption include, but are not limited to, monostearates and gelatin.

Diluents include, but are not limited to, water, aqueous buffers (such as buffered saline), alcohols and polyols (such as glycerol), and the like. Preservatives include, but are not limited to, various antibacterial and antifungal agents, such as thimerosal, 2-phenoxyethanol, parabens, chlorobutanol, phenol, sorbic acid, and the like. Stabilizer has the meaning generally understood by those skilled in the art, and it can stabilize the desired activity of the active ingredient in the medicine, including but not limited to sodium glutamate, gelatin, SPGA, sugars (such as sorbitol, mannitol, starch, sucrose, lactose, dextran, or glucose), amino acids (such as glutamic acid, glycine), proteins (such as dry whey, albumin or casein) or their degradation products (such as lactalbumin hydrolyzate), etc.

The terms “including”, “comprising”, “having”, “containing” or “involving” and other variations thereof herein are inclusive or open-ended and do not exclude other unlisted elements or method steps.

The term “effective amount” refers to the amount sufficient to achieve a desired clinical effect in an individual being treated. For instance, this may be the amount necessary to alleviate any particular disease symptom or inhibit or reduce the severity of a disease, or its complications in an individual. Determining such an effective amount is well within the capability of those skilled in the art. For example, amounts effective for therapeutic use will depend on the severity of the disease to be treated, the general state of the patient's own immune system, the general condition of the patient such as age, weight and sex, the mode of administration of the drug, and other treatments administered concomitantly etc. A therapeutically effective amount of an ADC may vary according to the following factors: The severity of the disease to be treated, the general state of the patient's own immune system, the general condition of the patient such as age, weight and sex, the mode of administration of the drug, and other treatments administered at the same time, etc.

The term “treatment” refers to a method implemented to obtain a beneficial or desired clinical outcome in a subject (human or animal individual) that is exhibiting a disease symptom or diagnosed as having a disease (“in need thereof”). For the purposes of this invention, a beneficial or desired clinical outcome includes, but is not limited to, relief of symptoms, reduction of the extent of the disease, stabilization (i.e., no longer worsening) of the disease state, delay or slowing of the progression of the disease, amelioration or palliation of the disease status, and relief of symptoms (whether partial or total), whether detectable or not. Additionally, “treating” can also refer to prolonging survival as compared to expected survival if not receiving treatment.

As used herein, the term “subject” refers to a mammal, such as a primate mammal, such as a human. In certain embodiments, the subject (e.g., a human) has a tumor, or is at risk of having a disease as described above.

The terms “cancer” and “tumor” are used interchangeably and refer to a broad class of diseases characterized by the uncontrolled growth of abnormal cells in the body. Unregulated cell division may lead to the formation of malignant tumors or cells that invade adjacent tissues and may metastasize to distant sites in the body through the lymphatic system or bloodstream. Cancer includes benign and malignant cancers as well as dormant tumors or micrometastases. Cancer also includes hematological malignancies.

The term “hematologic malignancies” includes lymphomas, leukemias, myelomas or lymphoid malignancies, as well as splenic and lymph node neoplasms. Exemplary lymphomas include B-cell lymphoma and T-cell lymphoma. B-cell lymphomas, including, for example, Hodgkin's lymphoma. T-cell lymphomas, including, for example, cutaneous T-cell lymphomas. Hematological malignancies also include leukemias, such as secondary leukemia or acute lymphoblastic leukemia. Hematological malignancies also include myeloma (e.g., multiple myeloma) and other hematological and/or B- or T-cell-related cancers.

The term “alkyl” means a group obtained by the loss of one hydrogen atom from a straight or branched hydrocarbon group, for example “C₁₋₂₀ alkyl”, “C₁₋₁₀ alkyl”, “C₁₋₆ alkyl”, “C₁₋₄ alkyl”, and “C₁₋₃ alkyl”. Specific examples include but are not limited to: methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc.

The term “alkylene” means a group obtained by the loss of two hydrogen atoms from a straight or branched hydrocarbon group, for example “C₁₋₂₀ alkylene”, “C₁₋₁₀ alkylene”, “C₃₋₁₀ alkylene”, “C₅₋₈ alkylene”, “C₁₋₆ alkylene”, “C₁₋₄ alkylene”, and “C₁₋₃ alkylene”. Specific examples include but are not limited to: methylene, ethylene, 1,3-propylene, 1,4-butylene, 1,5-pentylene, 1,6-hexylene, or the like.

The term “alkenylene” refers to a divalent group obtained by losing two hydrogen atoms from a linear or branched hydrocarbon group containing at least one carbon-carbon double bond, including, for example, “C₂₋₂₀ alkenylene”, “C₃₋₁₀ alkenylene”, “C₅₋₈ alkenylene”, etc. Examples include, but are not limited to: ethenylene, 1-propenylene, 2-propenylene, 1-butenylene, 2-butenylene, 1,3-butadienylene, 1-pentenylene, 2-pentenylene, 3-pentenylene, 1,3-pentadienylene, 1,4-pentadienylene, 1-hexenylene, 2-hexenylene, 3-hexenylene, 1,4-hexadienylene and the like.

The term “alkynylene” refers to a divalent group obtained by the loss of two hydrogen atoms from a linear or branched hydrocarbon group containing at least one carbon-carbon triple bond, including, for example, “C₂₋₂₀ alkynylene”, “C₃₋₁₀ alkynylene”, and “C₅₋₈ alkynylene”. Examples include, but are not limited to: ethynylene, 1-propynylene, 2-propynylene, 1-butynylene, 2-butynylene, 1,3-butadiynylene, 1-pentynyl, 2-pentynyl, 3-pentynyl, 1,3-pentadiynylene, 1,4-pentadiynylene, 1-hexynylene, 2-hexynylene, 3-hexynylene, 1,4-hexadiynylene, etc.

The term “heteroalicyclic ring” refers to a saturated or partially saturated cyclic structure containing at least one ring member selected from N, O and S. Specific examples include, but are not limited to, 5-6 membered aliphatic heterocyclic rings, 5-6 membered nitrogen-containing aliphatic heterocyclic rings, 5-6 membered oxygen-containing aliphatic heterocyclic rings, etc., for example tetrahydrofuran, pyrrolidine, piperidine, tetrahydropyran, etc.

The term “heteroaromatic ring” refers to an aromatic ring structure containing at least one ring member selected from N, O and S. Specific examples include but are not limited to 5-6 membered aromatic heterocyclic rings, 5-6 membered nitrogen-containing aromatic heterocyclic rings, 5-6 membered oxygen-containing aromatic heterocyclic rings, etc., for example furan, thiophene, pyrrole, thiazole, isothiazole, thiadiazole, oxazole, isoxazole, oxadiazole, imidazole, pyrazole, 1,2,3-triazole, 1,2,4-triazole, 1,2,3-oxadiazole, 1,2,4-oxadiazole, 1,2,5-oxadiazole, 1,3,4-oxadiazole, pyridine, pyrimidine, pyridazine, pyrazine, 1,2,3-triazine, 1,3,5-triazine, 1,2,4,5-tetrazine, etc.

The term “aromatic ring system” refers to a monocyclic or polycyclic ring system comprising at least one aromatic ring (like benzene ring) or heteroaromatic ring (like pyrimidine ring), two or more aromatic rings and/or heteroaromatic aromatic rings can form fused rings or be connected by single bonds (for example dipyrimidinylphenyl, etc.), and the aromatic ring system can be divalent or higher valence state (like trivalent or tetravalent), for example 5-20 membered aromatic ring system.

As used herein, “Cit” is abbreviation for Citrulline.

As used herein, the term “about” or “approximately” when used in conjunction with a numerical variable generally means that the value of the variable is within experimental error (e.g., within a 95% confidence interval for the mean) or within ±10%.

It should be noted that if there is a discrepancy between a depicted structure and a name for that structure, the depicted structure is to be accorded more weight.

### Antibody-Drug Conjugate

In one aspect, the present application provides an antibody-drug conjugate having a structure shown as a formula Ab-[M-L-E-D]ₓ, where:
Ab is an antibody or antigen-binding fragment thereof that specifically binds to human tyrosine kinase 7 (PTK7);
M is a linker site linked to the antibody or antigen-binding fragment thereof;
L is a linker between M and E;
E is a structural fragment linking L and D;
D is a cytotoxic drug fragment; and
x is selected from 1 to 10.

In some embodiments, the antibody or antigen-binding fragment thereof comprises:
(1) the following heavy chain variable regions (VH) and/or light chain variable regions (VL), wherein CDRs are defined according to a Chothia numbering system:
(1a) a heavy chain variable region (VH) comprising the following 3 CDRs: CDR-H1 having the amino acid sequence as set forth in SEQ ID NO: 11 or a variant thereof, CDR-H2 having the amino acid sequence as set forth in SEQ ID NO: 12 or a variant thereof, and CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(1b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (1a) and (1b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example replacement, deletion or addition of 1, 2 or 3 amino acids) compared with the sequence from which the variants are derived; preferably, the replacement is conservative replacement;
   or,
(2) the following heavy chain variable region (VH) and/or light chain variable region (VL), wherein the CDRs are defined according to the Kabat numbering system:
(2a) a heavy chain variable region (VH) comprising the following 3 CDRs: CDR-H1 having the amino acid sequence as set forth in SEQ ID NO: 17 or a variant thereof, CDR-H2 having the amino acid sequence as set forth in SEQ ID NO: 18 or 19 or a variant thereof, CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(2b) a heavy chain variable region (VH) comprising the following 3 CDRs: CDR-H1 having the amino acid sequence as set forth in SEQ ID NO: 33 or a variant thereof, CDR-H2 having the amino acid sequence as set forth in SEQ ID NO: 34 or 35 or a variant thereof, and CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (2a) and (2b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement;
   or,
(3) the following heavy chain variable regions (VH) and/or light chain variable regions (VL), wherein the CDRs are defined according to the IMGT numbering system:
(3a) a heavy chain variable region (VH) comprising the following 3 CDRs: CDR-H1 having the amino acid sequence as set forth in SEQ ID NO: 20 or a variant thereof, CDR-H2 having the amino acid sequence as set forth in SEQ ID NO: 21 or a variant thereof, and CDR-H3 having the amino acid sequence as set forth in SEQ ID NO: 22 or a variant thereof; and/or, a light chain variable region (VL) comprising the following 3 CDRs: CDR-L1 having the amino acid sequence as set forth in SEQ ID NO: 23 or a variant thereof, CDR-L2 having the amino acid sequence as set forth in SEQ ID NO: 24 or a variant thereof, and CDR-L3 having the amino acid sequence as set forth in SEQ ID NO: 16 or a variant thereof; or, (3b) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, and CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 39 or a variant thereof, CDR-L2 having the amino acid sequence as set forth in SEQ ID NO: 40 or a variant thereof, and CDR-L3 having the amino acid sequence as set forth in SEQ ID NO: 32 or a variant thereof;
where the variants in any one of (3a) and (3b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement;
   or,
(4) the following heavy chain variable regions (VH) and/or light chain variable regions (VL), wherein the CDRs are defined according to the AbM numbering system:
(4a) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, and CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(4b) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, and CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (4a) and (4b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement.

In some embodiments, the antibody or antigen-binding fragment thereof comprises:
(1a) a heavy chain variable region (VH) comprising the following 3 CDRs: CDR-H1 having the amino acid sequence as set forth in SEQ ID NO: 11 or a variant thereof, CDR-H2 having the amino acid sequence as set forth in SEQ ID NO: 12 or a variant thereof, and CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(1b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (1a) and (1b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example replacement, deletion or addition of 1, 2 or 3 amino acids) compared with the sequence from which the variants are derived; preferably, the replacement is conservative replacement;
   or,
(2a) a heavy chain variable region (VH) comprising the following 3 CDRs: CDR-H1 having the amino acid sequence as set forth in SEQ ID NO: 17 or a variant thereof, CDR-H2 having the amino acid sequence as set forth in SEQ ID NO: 18 or 19 or a variant thereof, CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(2b) a heavy chain variable region (VH) comprising the following 3 CDRs: CDR-H1 having the amino acid sequence as set forth in SEQ ID NO: 33 or a variant thereof, CDR-H2 having the amino acid sequence as set forth in SEQ ID NO: 34 or 35 or a variant thereof, and CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (2a) and (2b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement;
   or,
(3a) a heavy chain variable region (VH) comprising the following 3 CDRs: CDR-H1 having the amino acid sequence as set forth in SEQ ID NO: 20 or a variant thereof, CDR-H2 having the amino acid sequence as set forth in SEQ ID NO: 21 or a variant thereof, and CDR-H3 having the amino acid sequence as set forth in SEQ ID NO: 22 or a variant thereof; and/or, a light chain variable region (VL) comprising the following 3 CDRs: CDR-L1 having the amino acid sequence as set forth in SEQ ID NO: 23 or a variant thereof, CDR-L2 having the amino acid sequence as set forth in SEQ ID NO: 24 or a variant thereof, and CDR-L3 having the amino acid sequence as set forth in SEQ ID NO: 16 or a variant thereof; or,
(3b) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, and CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 39 or a variant thereof, CDR-L2 having the amino acid sequence as set forth in SEQ ID NO: 40 or a variant thereof, and CDR-L3 having the amino acid sequence as set forth in SEQ ID NO: 32 or a variant thereof;
where the variants in any one of (3a) and (3b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement;
   or,
(4a) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, and CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(4b) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, and CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (4a) and (4b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement.

In some embodiments, the antibody or antigen-binding fragment thereof comprises:
(1a) a heavy chain variable region (VH) comprising the following 3 CDRs: CDR-H1 having the amino acid sequence as set forth in SEQ ID NO: 11, CDR-H2 having the amino acid sequence as set forth in SEQ ID NO: 12, and CDR-H3 having the amino acid sequence as set forth in SEQ ID NO: 13; and/or, a light chain variable region (VL) comprising the following 3 CDRs: CDR-L1 having the amino acid sequence as set forth in SEQ ID NO: 14, CDR-L2 having the amino acid sequence as set forth in SEQ ID NO: 15, and CDR-L3 having the amino acid sequence as set forth in SEQ ID NO: 16; or,
(1b) a heavy chain variable region (VH) comprising the following 3 CDRs: CDR-H1 having the amino acid sequence as set forth in SEQ ID NO: 27, CDR-H2 having the amino acid sequence as set forth in SEQ ID NO: 28, CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, and CDR-L3 having the amino acid sequence as set forth in SEQ ID NO: 32;
   or,
(2a) a heavy chain variable region (VH) comprising the following 3 CDRs: CDR-H1 having the amino acid sequence as set forth in SEQ ID NO: 17, CDR-H2 having the amino acid sequence as set forth in SEQ ID NO: 18 or 19, CDR-H3 having the amino acid sequence as set forth in SEQ ID NO: 13; and/or, a light chain variable region (VL) comprising the following 3 CDRs: CDR-L1 having the amino acid sequence as set forth in SEQ ID NO: 14, CDR-L2 having the amino acid sequence as set forth in SEQ ID NO: 15, and CDR-L3 having the amino acid sequence as set forth in SEQ ID NO: 16; or,
(2b) a heavy chain variable region (VH) comprising the following 3 CDRs: CDR-H1 having the amino acid sequence as set forth in SEQ ID NO: 33, CDR-H2 having the amino acid sequence as set forth in SEQ ID NO: 34 or 35, and CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, and CDR-L3 having the amino acid sequence as set forth in SEQ ID NO: 32;
   or,
(3a) a heavy chain variable region (VH) comprising the following 3 CDRs: CDR-H1 having the amino acid sequence as set forth in SEQ ID NO: 20, CDR-H2 having the amino acid sequence as set forth in SEQ ID NO: 21, and CDR-H3 having the amino acid sequence as set forth in SEQ ID NO: 22; and/or, a light chain variable region (VL) comprising the following 3 CDRs: CDR-L1 having the amino acid sequence as set forth in SEQ ID NO: 23, CDR-L2 having the amino acid sequence as set forth in SEQ ID NO: 24, and CDR-L3 having the amino acid sequence as set forth in SEQ ID NO: 16; or,
(3b) a heavy chain variable region (VH) comprising the following 3 CDRs: CDR-H1 having the amino acid sequence as set forth in SEQ ID NO: 36, CDR-H2 having the amino acid sequence as set forth in SEQ ID NO: 37, and CDR-H3 having the amino acid sequence as set forth in SEQ ID NO: 38; and/or, a light chain variable region (VL) comprising the following 3 CDRs: CDR-L1 having the amino acid sequence as set forth in SEQ ID NO: 39, CDR-L2 having the amino acid sequence as set forth in SEQ ID NO: 40, and CDR-L3 having the amino acid sequence as set forth in SEQ ID NO: 32;
   or,
(4a) a heavy chain variable region (VH) comprising the following 3 CDRs: CDR-H1 having the amino acid sequence as set forth in SEQ ID NO: 25, CDR-H2 having the amino acid sequence as set forth in SEQ ID NO: 26, and CDR-H3 having the amino acid sequence as set forth in SEQ ID NO: 13; and/or, a light chain variable region (VL) comprising the following 3 CDRs: CDR-L1 having the amino acid sequence as set forth in SEQ ID NO: 14, CDR-L2 having the amino acid sequence as set forth in SEQ ID NO: 15, and CDR-L3 having the amino acid sequence as set forth in SEQ ID NO: 16; or,
(4b) a heavy chain variable region (VH) comprising the following 3 CDRs: CDR-H1 having the amino acid sequence as set forth in SEQ ID NO: 41, CDR-H2 having the amino acid sequence as set forth in SEQ ID NO: 42, and CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, and CDR-L3 having the amino acid sequence as set forth in SEQ ID NO: 32.

In some embodiments, the antibody or antigen-binding fragment thereof comprises:
(1a) a heavy chain variable region (VH) comprising the following 3 CDRs: CDR-H1 having the amino acid sequence as set forth in SEQ ID NO: 11, CDR-H2 having the amino acid sequence as set forth in SEQ ID NO: 12, and CDR-H3 having the amino acid sequence as set forth in SEQ ID NO: 13; and/or, a light chain variable region (VL) comprising the following 3 CDRs: CDR-L1 having the amino acid sequence as set forth in SEQ ID NO: 14, CDR-L2 having the amino acid sequence as set forth in SEQ ID NO: 15, and CDR-L3 having the amino acid sequence as set forth in SEQ ID NO: 16.

In some embodiments, the antibody or antigen-binding fragment thereof comprises:
(1b) a heavy chain variable region (VH) comprising the following 3 CDRs: CDR-H1 having the amino acid sequence as set forth in SEQ ID NO: 27, CDR-H2 having the amino acid sequence as set forth in SEQ ID NO: 28, CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, and CDR-L3 having the amino acid sequence as set forth in SEQ ID NO: 32.

In some embodiments, the antibody or antigen-binding fragment thereof comprises:
(2a) a heavy chain variable region (VH) comprising the following 3 CDRs: CDR-H1 having the amino acid sequence as set forth in SEQ ID NO: 17, CDR-H2 having the amino acid sequence as set forth in SEQ ID NO: 18 or 19, CDR-H3 having the amino acid sequence as set forth in SEQ ID NO: 13; and/or, a light chain variable region (VL) comprising the following 3 CDRs: CDR-L1 having the amino acid sequence as set forth in SEQ ID NO: 14, CDR-L2 having the amino acid sequence as set forth in SEQ ID NO: 15, and CDR-L3 having the amino acid sequence as set forth in SEQ ID NO: 16.

In some embodiments, the antibody or antigen-binding fragment thereof comprises:
(2b) a heavy chain variable region (VH) comprising the following 3 CDRs: CDR-H1 having the amino acid sequence as set forth in SEQ ID NO: 33, CDR-H2 having the amino acid sequence as set forth in SEQ ID NO: 34 or 35, and CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, and CDR-L3 having the amino acid sequence as set forth in SEQ ID NO: 32.

In some embodiments, the antibody or antigen-binding fragment thereof comprises:
(3a) a heavy chain variable region (VH) comprising the following 3 CDRs: CDR-H1 having the amino acid sequence as set forth in SEQ ID NO: 20, CDR-H2 having the amino acid sequence as set forth in SEQ ID NO: 21, and CDR-H3 having the amino acid sequence as set forth in SEQ ID NO: 22; and/or, a light chain variable region (VL) comprising the following 3 CDRs: CDR-L1 having the amino acid sequence as set forth in SEQ ID NO: 23, CDR-L2 having the amino acid sequence as set forth in SEQ ID NO: 24, and CDR-L3 having the amino acid sequence as set forth in SEQ ID NO: 16.

In some embodiments, the antibody or antigen-binding fragment thereof comprises:
(3b) a heavy chain variable region (VH) comprising the following 3 CDRs: CDR-H1 having the amino acid sequence as set forth in SEQ ID NO: 36, CDR-H2 having the amino acid sequence as set forth in SEQ ID NO: 37, and CDR-H3 having the amino acid sequence as set forth in SEQ ID NO: 38; and/or, a light chain variable region (VL) comprising the following 3 CDRs: CDR-L1 having the amino acid sequence as set forth in SEQ ID NO: 39, CDR-L2 having the amino acid sequence as set forth in SEQ ID NO: 40, and CDR-L3 having the amino acid sequence as set forth in SEQ ID NO: 32.

In some embodiments, the antibody or antigen-binding fragment thereof comprises:
(4a) a heavy chain variable region (VH) comprising the following 3 CDRs: CDR-H1 having the amino acid sequence as set forth in SEQ ID NO: 25, CDR-H2 having the amino acid sequence as set forth in SEQ ID NO: 26, and CDR-H3 having the amino acid sequence as set forth in SEQ ID NO: 13; and/or, a light chain variable region (VL) comprising the following 3 CDRs: CDR-L1 having the amino acid sequence as set forth in SEQ ID NO: 14, CDR-L2 having the amino acid sequence as set forth in SEQ ID NO: 15, and CDR-L3 having the amino acid sequence as set forth in SEQ ID NO: 16.

In some embodiments, the antibody or antigen-binding fragment thereof comprises:
(4b) a heavy chain variable region (VH) comprising the following 3 CDRs: CDR-H1 having the amino acid sequence as set forth in SEQ ID NO: 41, CDR-H2 having the amino acid sequence as set forth in SEQ ID NO: 42, and CDR-H3 having the amino acid sequence as set forth in SEQ ID NO: 29; and/or, a light chain variable region (VL) comprising the following 3 CDRs: CDR-L1 having the amino acid sequence as set forth in SEQ ID NO: 30, CDR-L2 having the amino acid sequence as set forth in SEQ ID NO: 31, and CDR-L3 having the amino acid sequence as set forth in SEQ ID NO: 32.

In some embodiments, the antibody or antigen-binding fragment thereof comprises:
(a) a VH having the amino acid sequence as set forth in SEQ ID NO: 1 or a variant thereof, and/or, a VL having the amino acid sequence as set forth in SEQ ID NO: 2 or a variant thereof;
(b) a VH having the amino acid sequence as set forth in SEQ ID NO: 3 or a variant thereof, and/or, a VL having the amino acid sequence as set forth in SEQ ID NO: 4 or a variant thereof;
(c) a VH having the amino acid sequence as set forth in SEQ ID NO: 5 or a variant thereof, and/or, a VL having the amino acid sequence as set forth in SEQ ID NO: 6 or a variant thereof;
(d) a VH having the amino acid sequence as set forth in SEQ ID NO: 7 or a variant thereof, and/or, a VL having the amino acid sequence as set forth in SEQ ID NO: 8 or a variant thereof; or
(e) a VH having the amino acid sequence as set forth in SEQ ID NO: 9 or a variant thereof, and/or, a VL having the amino acid sequence as set forth in SEQ ID NO: 10 or a variant thereof;
wherein the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (for example replacement, deletion or addition of 1, 2, 3, 4 or 5 amino acids) as compared to the sequence from which the variants are derived; and preferably, the replacement is conservative replacement.

In some embodiments, the antibody or antigen-binding fragment thereof comprises:
(a) a VH having the amino acid sequence as set forth in SEQ ID NO: 1, and/or, a VL having the amino acid sequence as set forth in SEQ ID NO: 2;
(b) a VH having the amino acid sequence as set forth in SEQ ID NO: 3, and/or, a VL having the amino acid sequence as set forth in SEQ ID NO: 4;
(c) a VH having the amino acid sequence as set forth in SEQ ID NO: 5, and/or, a VL having the amino acid sequence as set forth in SEQ ID NO: 6;
(d) a VH having the amino acid sequence as set forth in SEQ ID NO: 7, and/or, a VL having the amino acid sequence as set forth in SEQ ID NO: 8; or
(e) a VH having the amino acid sequence as set forth in SEQ ID NO: 9, and/or, a VL having the amino acid sequence as set forth in SEQ ID NO: 10.

In some embodiments, the antibody or antigen-binding fragment thereof comprises:
(a) a VH having the amino acid sequence as set forth in SEQ ID NO: 1, and/or, a VL having the amino acid sequence as set forth in SEQ ID NO: 2.

In some embodiments, the antibody or antigen-binding fragment thereof comprises:
(b) a VH having the amino acid sequence as set forth in SEQ ID NO: 3, and/or, a VL having the amino acid sequence as set forth in SEQ ID NO: 4.

In some embodiments, the antibody or antigen-binding fragment thereof comprises:
(c) a VH having the amino acid sequence as set forth in SEQ ID NO: 5, and/or, a VL having the amino acid sequence as set forth in SEQ ID NO: 6.

In some embodiments, the antibody or antigen-binding fragment thereof comprises:
(d) a VH having the amino acid sequence as set forth in SEQ ID NO: 7, and/or, a VL having the amino acid sequence as set forth in SEQ ID NO: 8.

In some embodiments, the antibody or antigen-binding fragment thereof comprises:
(e) a VH having the amino acid sequence as set forth in SEQ ID NO: 9, and/or, a VL having the amino acid sequence as set forth in SEQ ID NO: 10.

In some embodiments, the antibody or antigen-binding fragment thereof comprises: a heavy chain variable region (VH) comprising a CDR-H1, a CDR-H2, and a CDR-H3 comprising amino acid sequences of the CDR-H1, the CDR-H2, and the CDR-H3 as set forth in the VH having the amino acid sequence of SEQ ID NO: 1, respectively; and a light chain variable region (VL) comprising a CDR-L1, a CDR-L2, and a CDR-L3 comprising amino acid sequences of the CDR-L1, the CDR-L2, and the CDR-L3 as set forth in the VL having the amino acid sequence of SEQ ID NO: 2, respectively.

In some embodiments, the antibody or antigen-binding fragment thereof comprises: a heavy chain variable region (VH) comprising a CDR-H1, a CDR-H2, and a CDR-H3 comprising amino acid sequences of the CDR-H1, the CDR-H2, and the CDR-H3 as set forth in the VH having the amino acid sequence of SEQ ID NO: 3, respectively; and a light chain variable region (VL) comprising a CDR-L1, a CDR-L2, and a CDR-L3 comprising amino acid sequences of the CDR-L1, the CDR-L2, and the CDR-L3 as set forth in the VL having the amino acid sequence of SEQ ID NO: 4, respectively.

In some embodiments, the antibody or antigen-binding fragment thereof comprises: a heavy chain variable region (VH) comprising a CDR-H1, a CDR-H2, and a CDR-H3 comprising amino acid sequences of the CDR-H1, the CDR-H2, and the CDR-H3 as set forth in the VH having the amino acid sequence of SEQ ID NO: 5, respectively; and a light chain variable region (VL) comprising a CDR-L1, a CDR-L2, and a CDR-L3 comprising amino acid sequences of the CDR-L1, the CDR-L2, and the CDR-L3 as set forth in the VL having the amino acid sequence of SEQ ID NO: 6, respectively.

In some embodiments, the antibody or antigen-binding fragment thereof comprises: a heavy chain variable region (VH) comprising a CDR-H1, a CDR-H2, and a CDR-H3 comprising amino acid sequences of the CDR-H1, the CDR-H2, and the CDR-H3 as set forth in the VH having the amino acid sequence of SEQ ID NO: 7, respectively; and a light chain variable region (VL) comprising a CDR-L1, a CDR-L2, and a CDR-L3 comprising amino acid sequences of the CDR-L1, the CDR-L2, and the CDR-L3 as set forth in the VL having the amino acid sequence of SEQ ID NO: 8, respectively.

In some embodiments, the antibody or antigen-binding fragment thereof comprises: a heavy chain variable region (VH) comprising a CDR-H1, a CDR-H2, and a CDR-H3 comprising amino acid sequences of the CDR-H1, the CDR-H2, and the CDR-H3 as set forth in the VH having the amino acid sequence as set forth in SEQ ID NO: 9, respectively; and a light chain variable region (VL) comprising a CDR-L1, a CDR-L2, and a CDR-L3 comprising amino acid sequences of the CDR-L1, the CDR-L2, and the CDR-L3 as set forth in the VL having the amino acid sequence of SEQ ID NO: 10, respectively.

In some embodiments, the antibody or antigen-binding fragment thereof further comprises:
(a) a human immunoglobulin heavy chain constant region (CH) or a variant thereof, wherein the variant has replacement, deletion or addition of one or more amino acids (for example, replacement, deletion or addition of up to 20, up to 15, up to 10 or up to 5 amino acids; for example, replacement, deletion or addition of 1, 2, 3, 4 or 5 amino acids) as compared to a wild-type sequence from which the variant is derived; and
(b) a human immunoglobulin light chain constant (CL) or a variant, wherein the variant has replacement, deletion or addition of one or more amino acids (for example replacement, deletion or addition of up to 20, up to 15, up to 10 or up to 5 amino acids; for example, replacement, deletion or addition of 1, 2, 3, 4 or 5 amino acids) as compared to the wild-type sequence from which the variant is derived.

In some embodiments, the heavy chain constant region is an IgG heavy chain constant region, for example an IgG1, IgG2, IgG3 or IgG4 heavy chain constant region, for example a human IgG1 heavy chain constant region or a human IgG4 heavy chain constant region. In some embodiments, the antibody or antigen-binding fragment thereof comprises a heavy chain constant region (CH) shown as SEQ ID NO: 43 or a variant thereof, and the variant has conservative replacement of up to 20 amino acids (for example, conservative replacement of up to 15, up to 10 or up to 5 amino acids; for example, conservative replacement of 1, 2, 3, 4 or 5 amino acids) as compared to SEQ ID NO: 43.

In some embodiments, the light chain constant region is a k light chain constant region. In some embodiments, the antibody or antigen-binding region thereof comprises a light chain constant region (CL) shown as SEQ ID NO: 44 or a variant thereof, and the variant has conservative replacement of up to 20 amino acids (for example, conservative replacement of up to 15, up to 10 or up to 5 amino acids; for example, conservative replacement of 1, 2, 3, 4 or 5 amino acids) as compared to SEQ ID NO: 44.

In some embodiments, the antibody or antigen-binding fragment thereof comprises a heavy chain constant region (CH) shown as SEQ ID NO: 43 or 45, and a light chain constant region (CL) shown as SEQ ID NO: 44.

In some embodiments, the antibody or antigen-binding fragment thereof comprises:
(1) a heavy chain comprising a VH having the amino acid sequence as set forth in SEQ ID NO: 1 and a heavy chain constant region (CH) shown as SEQ ID NO: 43, and, a light chain comprising a VL having the amino acid sequence as set forth in SEQ ID NO: 2 and a light chain constant region (CL) having the amino acid sequence as set forth in SEQ ID NO: 44;
(2) a heavy chain comprising a VH having the amino acid sequence as set forth in SEQ ID NO: 3 and a heavy chain constant region (CH) having the amino acid sequence as set forth in SEQ ID NO: 43 or 45, and, a light chain comprising a VL having the amino acid sequence as set forth in SEQ ID NO: 4 and a light chain constant region (CL) having the amino acid sequence as set forth in SEQ ID NO: 44;
(3) a heavy chain comprising a VH having the amino acid sequence as set forth in SEQ ID NO: 5 and a heavy chain constant region (CH) having the amino acid sequence as set forth in SEQ ID NO: 43, and, a light chain comprising a VL having the amino acid sequence as set forth in SEQ ID NO: 6 and a light chain constant region (CL) having the amino acid sequence as set forth in SEQ ID NO: 44;
(4) a heavy chain comprising a VH having the amino acid sequence as set forth in SEQ ID NO: 7 and a heavy chain constant region (CH) having the amino acid sequence as set forth in SEQ ID NO: 43, and, a light chain comprising a VL having the amino acid sequence as set forth in SEQ ID NO: 8 and a light chain constant region (CL) having the amino acid sequence as set forth in SEQ ID NO: 44; or
(5) a heavy chain comprising a VH having the amino acid sequence as set forth in SEQ ID NO: 9 and a heavy chain constant region (CH) having the amino acid sequence as set forth in SEQ ID NO: 43, and, a light chain comprising a VL sequence having the amino acid sequence as set forth in SEQ ID NO: 10 and a light chain constant region (CL) having the amino acid sequence as set forth in SEQ ID NO: 44.

In some embodiments, the antibody or antigen-binding fragment thereof comprises:
(1) a heavy chain comprising a VH having the amino acid sequence as set forth in SEQ ID NO: 1 and heavy chain constant region (CH) having the amino acid sequence as set forth in SEQ ID NO: 43, and, a light chain comprising a VL having the amino acid sequence as set forth in SEQ ID NO: 2 and light chain constant region (CL) having the amino acid sequence as set forth in SEQ ID NO: 44.

In some embodiments, the antibody or antigen-binding fragment thereof comprises:
(2) a heavy chain comprising a VH having the amino acid sequence as set forth in SEQ ID NO: 3 and heavy chain constant region (CH) having the amino acid sequence as set forth in SEQ ID NO: 43 or 45, and, a light chain comprising a VL having the amino acid sequence as set forth in SEQ ID NO: 4 and light chain constant region (CL) having the amino acid sequence as set forth in SEQ ID NO: 44.

In some embodiments, the antibody or antigen-binding fragment thereof comprises:
(3) a heavy chain comprising a VH having the amino acid sequence as set forth in SEQ ID NO: 5 and heavy chain constant region (CH) having the amino acid sequence as set forth in SEQ ID NO: 43, and, a light chain comprising a VL having the amino acid sequence as set forth in SEQ ID NO: 6 and light chain constant region (CL) having the amino acid sequence as set forth in SEQ ID NO: 44.

In some embodiments, the antibody or antigen-binding fragment thereof comprises:
(4) a heavy chain comprising a VH having the amino acid sequence as set forth in SEQ ID NO: 7 and heavy chain constant region (CH) having the amino acid sequence as set forth in SEQ ID NO: 43, and, a light chain comprising a VL shown as SEQ ID NO: 8 and light chain constant region (CL) shown as SEQ ID NO: 44.

In some embodiments, the antibody or antigen-binding fragment thereof comprises:
(5) a heavy chain comprising a VH shown as SEQ ID NO: 9 and heavy chain constant region (CH) shown as SEQ ID NO: 43, and, a light chain comprising a VL shown as SEQ ID NO: 10 and light chain constant region (CL) shown as SEQ ID NO: 44.

In some embodiments, the antibody or antigen-binding fragment thereof comprises a heavy chain (HC) having the amino acid sequence as set forth in SEQ ID NO: 46, and a light chain (LC) having the amino acid sequence as set forth in SEQ ID NO: 47.

In certain embodiments, the antibody or antigen-binding fragment thereof comprises a heavy chain (HC) having the amino acid sequence as set forth in SEQ ID NO: 52, and a light chain (LC) having the amino acid sequence as set forth in SEQ ID NO: 47.

In certain embodiments, the antibody or antigen-binding fragment thereof comprises a heavy chain (HC) having the amino acid sequence as set forth in SEQ ID NO: 53, and a light chain (LC) having the amino acid sequence as set forth in SEQ ID NO: 47.

In certain embodiments, the antibody or antigen-binding fragment thereof comprises a heavy chain (HC) having the amino acid sequence as set forth in SEQ ID NO: 54, and a light chain (LC) having the amino acid sequence as set forth in SEQ ID NO: 47.

In certain embodiments of antibody or antibody-drug conjugate disclosed herein, the heavy chain constant domains may comprise a C-terminal lysine or lack either a C-terminal lysine or a C-terminal glycine-lysine dipeptide. In certain embodiments of the antibody or antibody-drug conjugate disclosed herein, the N-terminal amino acid of the antibody variable domains may undergo cyclization to pyroglutamate.

In certain embodiments of the antibody or antibody-drug conjugate disclosed herein, the N-terminal amino acid of the antibody variable domains may undergo cyclization to pyroglutamic acid.

In certain embodiments, the antibody or antigen-binding fragment disclosed herein includes the antibody or antigen-binding fragment that specifically binds the antigen, and may include post-translational modifications thereof (e.g., C-terminal Lysine clipping in the heavy chain, conversion of glutamine or glutamic acid to pyroglutamate or pyroglutamic acid) which may occur when recombinantly expressed in host cells (e.g., CHO cells), or during purification/storage.

Thus, a composition may comprise a population of antibody-drug conjugate species wherein each species may independently comprise a C-terminal lysine, lack a C-terminal lysine, lack a C-terminal glycine-lysine and/or comprise an N-terminal glutamine or glutamic acid or cyclization of the N-terminal amino acid to pyroglutamate.

In certain embodiments, the composition may comprise a population of antibody-drug conjugate species wherein each species may independently comprise a C-terminal lysine, lack a C-terminal lysine, lack a C-terminal glycine-lysine and/or comprise an N-terminal glutamine or glutamic acid or cyclization of the N-terminal amino acid to pyroglutamic acid.

Therefore, in particular embodiments, the present invention further provides compositions comprising an ADC disclosed herein wherein the predominant ADC species in the composition comprises (i) antibodies in which the heavy chain C-terminus lacks a lysine residue; (ii) antibodies in which the heavy chain N-terminus is glutamine, glutamic acid, pyroglutamate or pyroglutamic acid; or, (iii) antibodies in which the heavy chain C-terminus lacks a lysine residue and the heavy chain N-terminus is glutamine, glutamic acid, pyroglutamate or pyroglutamic acid.

Therefore, in particular embodiments, the present invention further provides compositions comprising an ADC disclosed herein wherein the predominant ADC species in the composition comprises antibodies in which the heavy chain C-terminus lacks a lysine residue and the heavy chain N-terminus is pyroglutamate or pyroglutamic acid.

In certain embodiments, the antibody or antigen-binding fragment thereof according to the present invention comprises a heavy chain comprising a VH having the amino acid sequence selected from the group consisting of SEQ ID NOs: 1, 3, 5, 7, and 9.

In certain embodiments, the antibody or antigen-binding fragment thereof according to the present invention comprises a heavy chain comprising a VH having the amino acid sequence set forth in SEQ ID NO: 1.

In certain embodiments, the antibody or antigen-binding fragment thereof according to the present invention comprises a heavy chain comprising a VH having the amino acid sequence set forth in SEQ ID NO: 3.

In certain embodiments, the antibody or antigen-binding fragment thereof according to the present invention comprises a heavy chain comprising a VH having the amino acid sequence set forth in SEQ ID NO: 5.

In certain embodiments, the antibody or antigen-binding fragment thereof according to the present invention comprises a heavy chain comprising a VH having the amino acid sequence set forth in SEQ ID NO: 7.

In certain embodiments, the antibody or antigen-binding fragment thereof according to the present invention comprises a heavy chain comprising a VH having the amino acid sequence set forth in SEQ ID NO: 9.

In certain embodiments, the antibody or antigen-binding fragment thereof according to the present invention comprises a heavy chain comprising a VH having the amino acid sequence selected from the group consisting of SEQ ID NOs: 1, 3, 5, 7 and 9, wherein the N-terminal glutamine or glutamic acid of the antibody heavy chain variable region has undergone cyclization to pyroglutamate.

In certain embodiments, the antibody or antigen-binding fragment thereof according to the present invention comprises a heavy chain comprising a VH having the amino acid sequence selected from the group consisting of SEQ ID NOs: 1, 3, 5, 7 and 9, wherein the N-terminal glutamine or glutamic acid of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 1, wherein the N-terminal glutamic acid of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 1, wherein the N-terminal glutamic acid of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 3, wherein the N-terminal glutamic acid of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 3, wherein the N-terminal glutamic acid of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 5, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 5, wherein the N-terminal glutamic acid of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 7, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 7, wherein the N-terminal glutamic acid of the antibody heavy chain variable region has undergone cyclization to pyroglutamic acid.

In certain embodiments, the antibody or antigen-binding fragment thereof according to the present invention comprises a heavy chain comprising a VH having the amino acid sequence set forth in SEQ ID NO: 9, wherein the N-terminal glutamine of the antibody heavy chain variable region has undergone cyclization to pyroglutamate.

In certain embodiments, the antibody or antigen-binding fragment thereof according to the present invention comprises a heavy chain comprising a VH having the amino acid sequence set forth in SEQ ID NO: 9, wherein the N-terminal glutamic acid of the antibody heavy chain variable region has undergone cyclization to pyroglutamic acid.

In certain embodiments, the antibody or antigen-binding fragment thereof according to the present invention comprises a light chain comprising the VL having the amino acid sequence set forth in SEQ ID NO: 8, wherein the N-terminal glutamic acid of the antibody light chain variable region has undergone cyclization to pyroglutamate.

In certain embodiments, the antibody or antigen-binding fragment thereof according to the present invention comprises a light chain comprising the VL having the amino acid sequence set forth in SEQ ID NO: 8, wherein the N-terminal glutamic acid of the antibody light chain variable region has undergone cyclization to pyroglutamic acid.

In certain embodiments, the antibody or antigen-binding fragment thereof according to the present invention comprises: a heavy chain comprising a VH having the amino acid sequence set forth in SEQ ID NO: 7 and/or a light chain comprising a VL having the amino acid sequence set forth in SEQ ID NO: 8, and wherein and the N-terminal glutamine of SEQ ID NO: 7 and/or the N-terminal glutamic acid of SEQ ID NO: 8 has undergone cyclization to pyroglutamate.

In certain embodiments, the antibody or antigen-binding fragment thereof according to the present invention comprises: a heavy chain comprising a VH having the amino acid sequence set forth in SEQ ID NO: 7 and/or a light chain comprising a VL having the amino acid sequence set forth in SEQ ID NO: 8, and wherein and the N-terminal glutamine of SEQ ID NO: 7 and/or the N-terminal glutamic acid of SEQ ID NO: 8 has undergone cyclization to pyroglutamic acid.

In certain embodiments, the antibody or antigen binding fragment comprises:
(a) a heavy chain consisting of the sequence shown as SEQ ID NO: 52 and a light chain consisting of the sequence shown as SEQ ID NO: 47;
(b) a heavy chain consisting of the sequence shown as SEQ ID NO: 53 and a light chain consisting of the sequence shown as SEQ ID NO: 47; or
(c) a heavy chain consisting of the sequence shown as SEQ ID NO: 54 and a light chain consisting of the sequence shown as SEQ ID NO: 47.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises:
(a) a VH having the amino acid sequence set forth in SEQ ID NO: 1 or a variant thereof and/or a VL having the amino acid sequence set forth in SEQ ID NO: 2 or a variant thereof;
(b) a VH having the amino acid sequence set forth in SEQ ID NO: 3 or a variant thereof and/or a VL having the amino acid sequence set forth in SEQ ID NO: 4 or a variant thereof;
(c) a VH having the amino acid sequence set forth in SEQ ID NO: 5 or a variant thereof and/or a VL having the amino acid sequence set forth in SEQ ID NO: 6 or a variant thereof;
(d) a VH having the amino acid sequence set forth in SEQ ID NO: 7 or a variant thereof and/or a VL having the amino acid sequence set forth in SEQ ID NO: 8 or a variant thereof; or
(e) a VH having the amino acid sequence set forth in SEQ ID NO: 9 or a variant thereof and/or a VL having the amino acid sequence set forth in SEQ ID NO: 10 or a variant thereof;
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived.

In certain embodiments, the replacement is conservative replacement. In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises:
(a) a VH having the amino acid sequence set forth in SEQ ID NO: 1 and/or a VL having the amino acid sequence set forth in SEQ ID NO: 2;
(b) a VH having the amino acid sequence set forth in SEQ ID NO: 3 and/or a VL having the amino acid sequence set forth in SEQ ID NO: 4;
(c) a VH having the amino acid sequence set forth in SEQ ID NO: 5 and/or a VL having the amino acid sequence set forth in SEQ ID NO: 6;
(d) a VH having the amino acid sequence set forth in SEQ ID NO: 7 and/or a VL having the amino acid sequence set forth in SEQ ID NO: 8; or
(e) a VH having the amino acid sequence set forth in SEQ ID NO: 9 and/or a VL having the amino acid sequence set forth in SEQ ID NO: 10.

In certain embodiments, the antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises:
(1) a heavy chain comprising VH sequence shown as SEQ ID NO: 1 and heavy chain constant region (CH) shown as SEQ ID NO: 43, and a light chain comprising VL sequence shown as SEQ ID NO: 2 and light chain constant region (CL) shown as SEQ ID NO: 44;
(2) a heavy chain comprising VH sequence shown as SEQ ID NO: 3 and heavy chain constant region (CH) shown as SEQ ID NO: 43 or 45, and a light chain comprising VL sequence shown as SEQ ID NO: 4 and light chain constant region (CL) shown as SEQ ID NO: 44;
(3) a heavy chain comprising VH sequence shown as SEQ ID NO: 5 and heavy chain constant region (CH) shown as SEQ ID NO: 43, and a light chain comprising VL sequence shown as SEQ ID NO: 6 and light chain constant region (CL) shown as SEQ ID NO: 44;
(4) a heavy chain comprising VH sequence shown as SEQ ID NO: 7 and heavy chain constant region (CH) shown as SEQ ID NO: 43, and a light chain comprising VL sequence shown as SEQ ID NO: 8 and light chain constant region (CL) shown as SEQ ID NO: 44;
(5) a heavy chain comprising VH sequence shown as SEQ ID NO: 9 and heavy chain constant region (CH) shown as SEQ ID NO: 43, and a light chain comprising VL sequence shown as SEQ ID NO: 10 and light chain constant region (CL) shown as SEQ ID NO: 44;
(6) a heavy chain (HC) having the amino acid sequence as set forth in SEQ ID NO: 46 and a light chain (LC) having the amino acid sequence as set forth in SEQ ID NO: 47;
(7) a VH having the amino acid sequence set forth in SEQ ID NO: 1 and a VL having the amino acid sequence set forth in SEQ ID NO: 2;
(8) a VH having the amino acid sequence set forth in SEQ ID NO: 3 and a VL having the amino acid sequence set forth in SEQ ID NO: 4;
(9) a VH having the amino acid sequence set forth in SEQ ID NO: 5 and a VL having the amino acid sequence set forth in SEQ ID NO: 6;
(10) a VH having the amino acid sequence set forth in SEQ ID NO: 7 and a VL having the amino acid sequence set forth in SEQ ID NO: 8; or
(11) a VH having the amino acid sequence set forth in SEQ ID NO: 9 and a VL having the amino acid sequence set forth in SEQ ID NO: 10.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (a) a VH having the amino acid sequence set forth in SEQ ID NO: 1 or a variant thereof and/or a VL having the amino acid sequence set forth in SEQ ID NO: 2 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (b) a VH having the amino acid sequence set forth in SEQ ID NO: 3 or a variant thereof and/or a VL having the amino acid sequence set forth in SEQ ID NO: 4 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (c) a VH having the amino acid sequence set forth in SEQ ID NO: 5 or a variant thereof and/or a VL having the amino acid sequence set forth in SEQ ID NO: 6 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (d) a VH having the amino acid sequence set forth in SEQ ID NO: 7 or a variant thereof and/or a VL having the amino acid sequence set forth in SEQ ID NO: 8 or a variant thereof.

In certain embodiments, the antibody or antigen-binding fragment thereof according to the present invention is obtainable by expressing the nucleic acid molecules encoding the heavy chains and the light chains of the antibody or antigen-binding fragment thereof in a host cell, wherein the antibody or antigen-binding fragment thereof comprises: (e) a VH having the amino acid sequence set forth in SEQ ID NO: 9 or a variant thereof and/or a VL having the amino acid sequence set forth in SEQ ID NO: 10 or a variant thereof.

In particular embodiments of the antibody, the heavy chain constant domains as disclosed herein may comprise a C-terminal lysine or lack either a C-terminal lysine or a C-terminal glycine-lysine dipeptide. In some embodiments of the antibody or antigen binding fragment thereof, the N-terminal amino acid of the antibody or antigen binding fragment thereof may undergo cyclization to pyroglutamate.

In some embodiments of the antibody or antigen binding fragment thereof, the N-terminal amino acid of the antibody or antigen binding fragment thereof may undergo cyclization to pyroglutamic acid.

Thus, in a composition comprising a particular antibody disclosed herein, the composition may comprise a population of antibody species wherein each species may independently comprise a C-terminal lysine, lack a C-terminal lysine, lack a C-terminal glycine-lysine and/or comprise an N-terminal glutamine or glutamic acid or cyclization of the N-terminal amino acid to pyroglutamate.

In certain embodiment, in a composition comprising a particular antibody disclosed herein, the composition may comprise a population of antibody species wherein each species may independently comprise a C-terminal lysine, lack a C-terminal lysine, lack a C-terminal glycine-lysine and/or comprise an N-terminal glutamine or glutamic acid or cyclization of the N-terminal amino acid to pyroglutamic acid.

In the antibody-drug conjugate, the cytotoxic drug may be linked to the antibody or antigen-binding fragment through the linker (like “M-L-E” fragment shown in the present application).

In some embodiments, M is

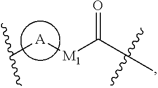

wherein ring A is a 5-6 membered aliphatic heterocyclic ring, or 5-20 membered aromatic ring system, and the aliphatic heterocyclic ring and aromatic ring systems are optionally substituted by one or more members selected from the group consisting of oxo (═O), halogen, cyano, amino, carboxyl, mercapto and C₁₋₆ alkyl; and M₁ is selected from a single bond, C₁₋₂₀ alkylene, C₂₋₂₀ alkenylene and C₂₋₂₀ alkynylene.

In some embodiments, M

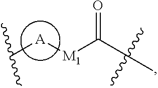

is wherein ring A is a 5-membered aliphatic heterocyclic ring, a 6-membered aromatic heterocyclic ring, or a polycyclic ring formed by linking more than one 6-membered aromatic heterocyclic ring and a benzene ring through a single bond, and the aliphatic heterocyclic ring is optionally substituted by one or more members selected from the group consisting of oxo (═O), halogen and C₁₋₄ alkyl; and M₁ is selected from a single bond, C₃₋₁₀ alkylene, C₃₋₁₀ alkenylene and C₃₋₁₀ alkynylene.

In some embodiments, M

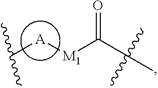

is wherein ring A is selected from

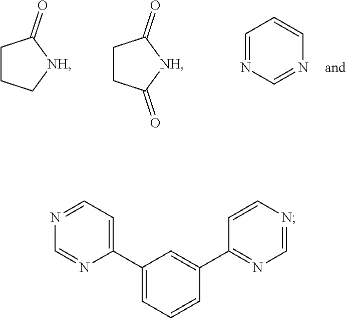 [SMILES: O=C1CCCN1 ; O=C1CCC(=O)N1 ; c1cncnc1 ; c1cc(-c2ccncn2)cc(-c2ccncn2)c1]

and M₁ is selected from a single bond, C₅₋₈ alkylene, C₅₋₈ alkenylene and C₅₋₈ alkynylene.

In some embodiments, M is selected from the following structures:

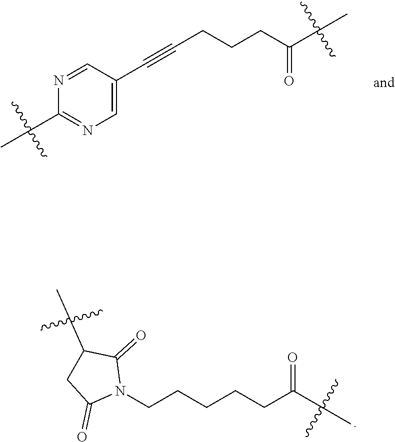 [SMILES: *C(=O)CCCC#Cc1cnc(*)nc1 ; *C(=O)CCCCCN1C(=O)CC(*)C1=O]

In some embodiments, M is selected from the following structures:

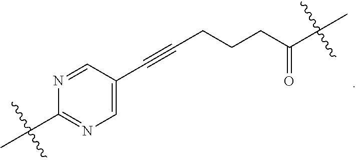 [SMILES: *C(=O)CCCC#Cc1cnc(*)nc1]

In some embodiments, L is selected from a structure comprising one or more of C₁₋₆ alkylene, —N(R′)—, carbonyl, —O—, Val, Cit, Phe, Lys, Lys(COCH₂CH₂(OCH₂CH₂)ₛOCH₃), D-Val, Leu, Gly, Ala, Asn, Val-Cit, Val-Ala, Val-Lys, Val-Lys(Ac), Phe-Lys, Phe-Lys(Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly (SEQ ID NO: 48), Gly-Phe-Leu-Gly (SEQ ID NO: 49), Gly-Gly-Val-Ala (SEQ ID NO: 50), Gly-Gly-Gly-Gly-Gly (SEQ ID NO: 51),

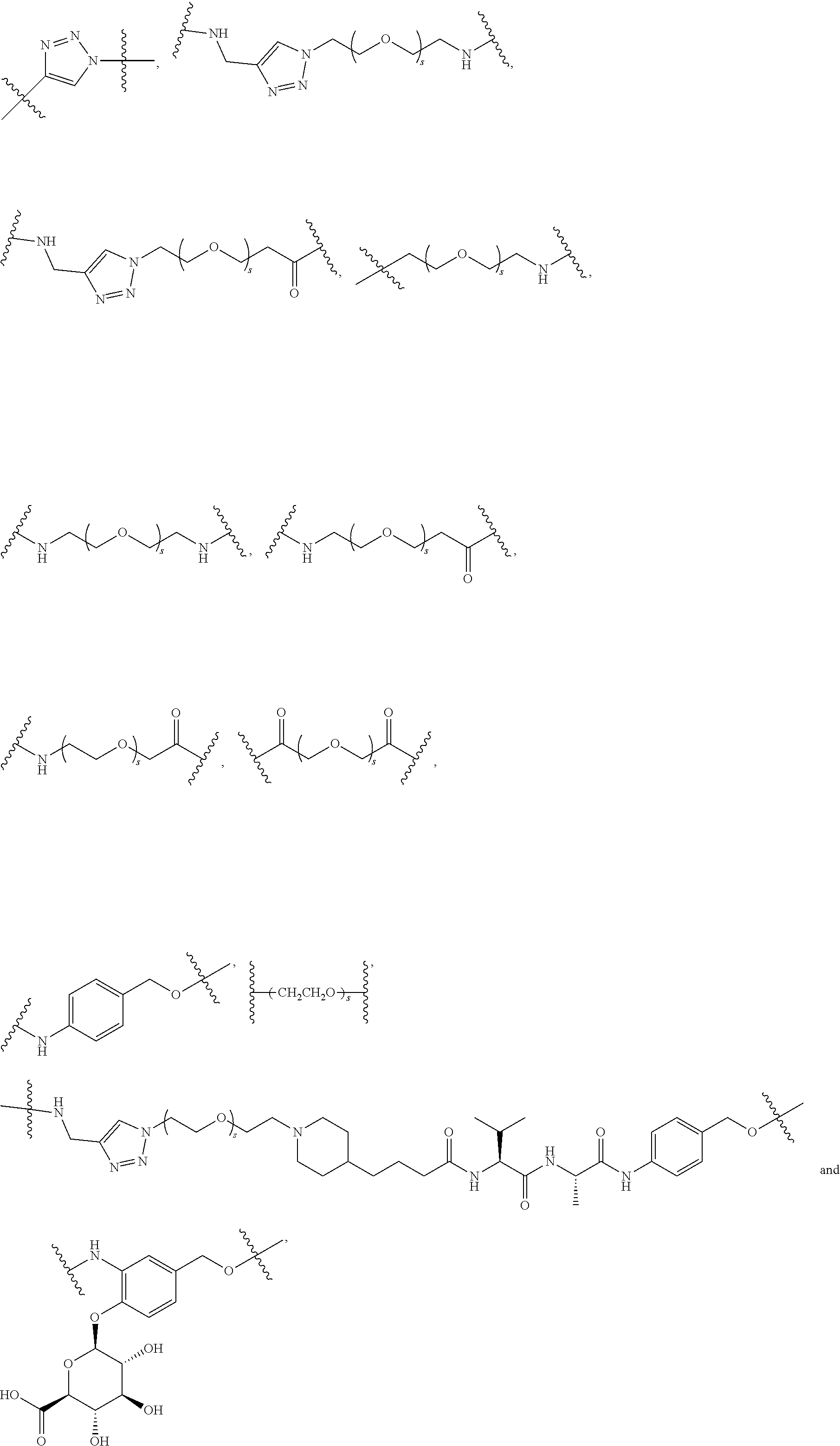

wherein R′ represents hydrogen, C₁₋₆ alkyl or alkyl containing —(CH₂CH₂O)r-; r is selected from an integer from 1-10; and s is selected from an integer from 1-20.

In some embodiments, L is selected from a structure comprising one or more of C₁₋₆ alkylene, —NH—, Val, Cit, Phe, Lys, Lys(COCH₂CH₂(OCH₂CH₂)ₛOCH₃), Gly, Val-Cit, Gly-Gly-Phe-Gly (SEQ ID NO: 48),

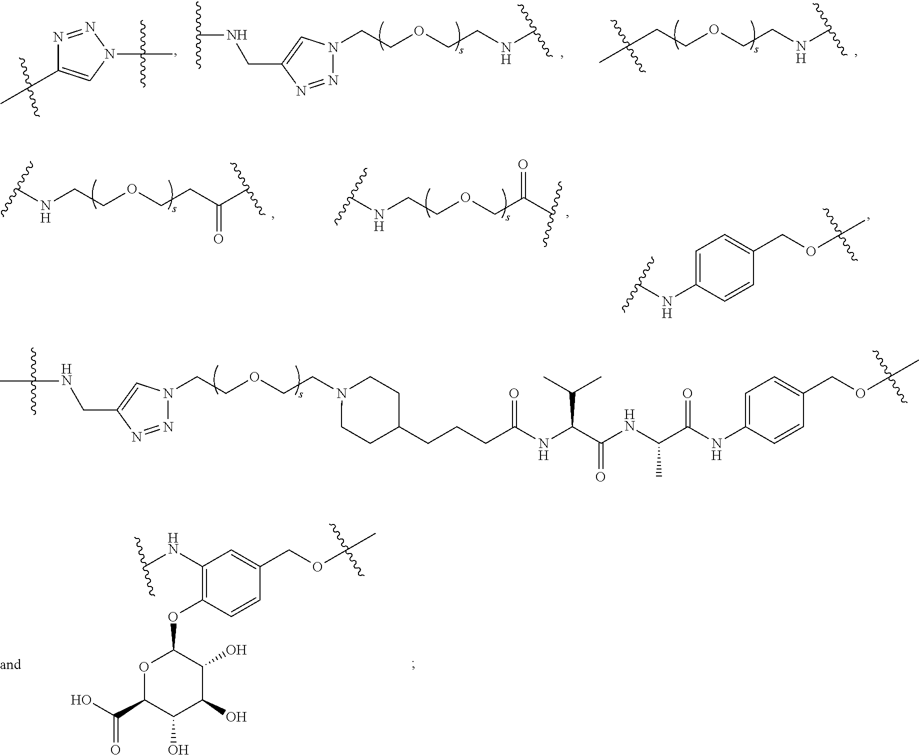

where s is selected from an integer from 1-20.

In some embodiments, L is selected from the following structures:

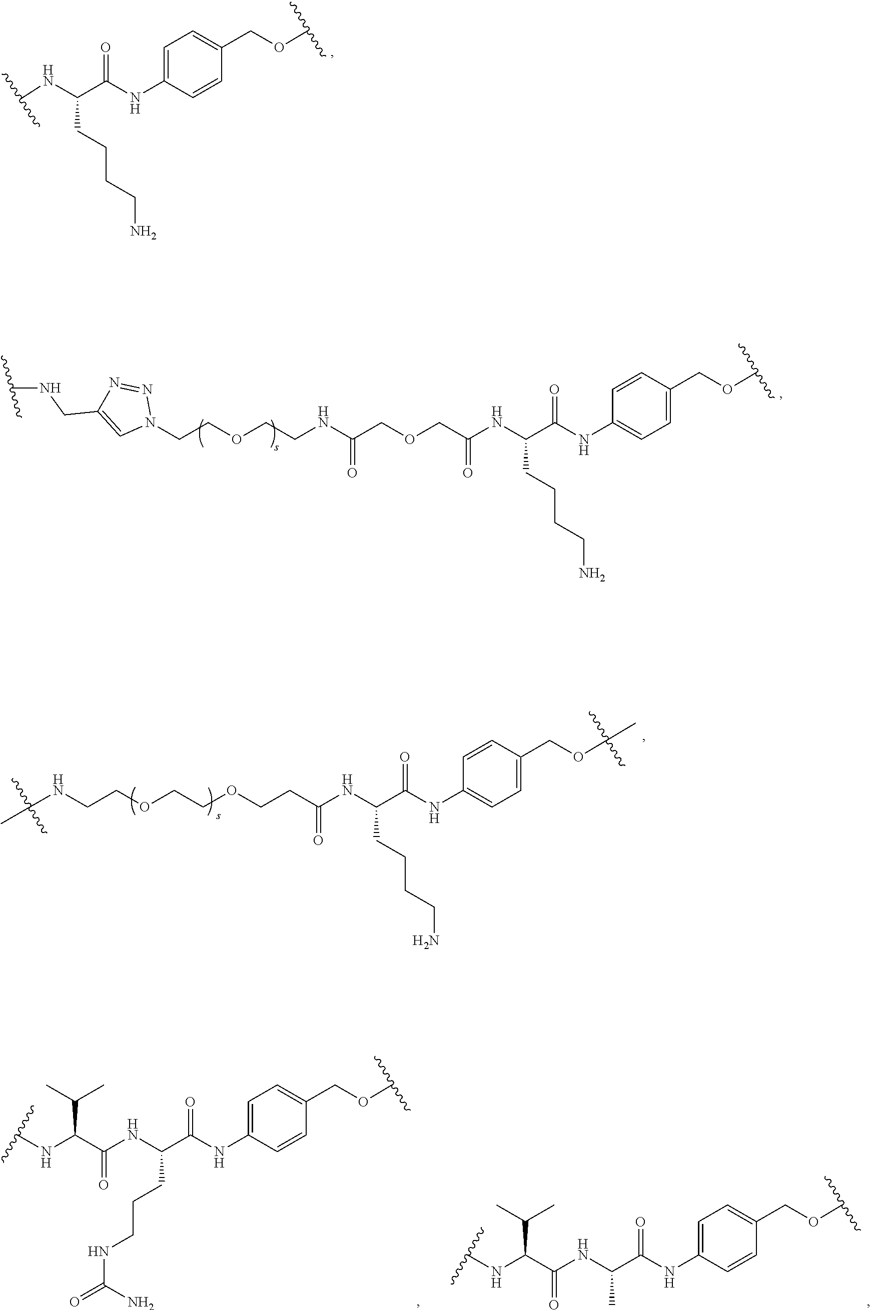

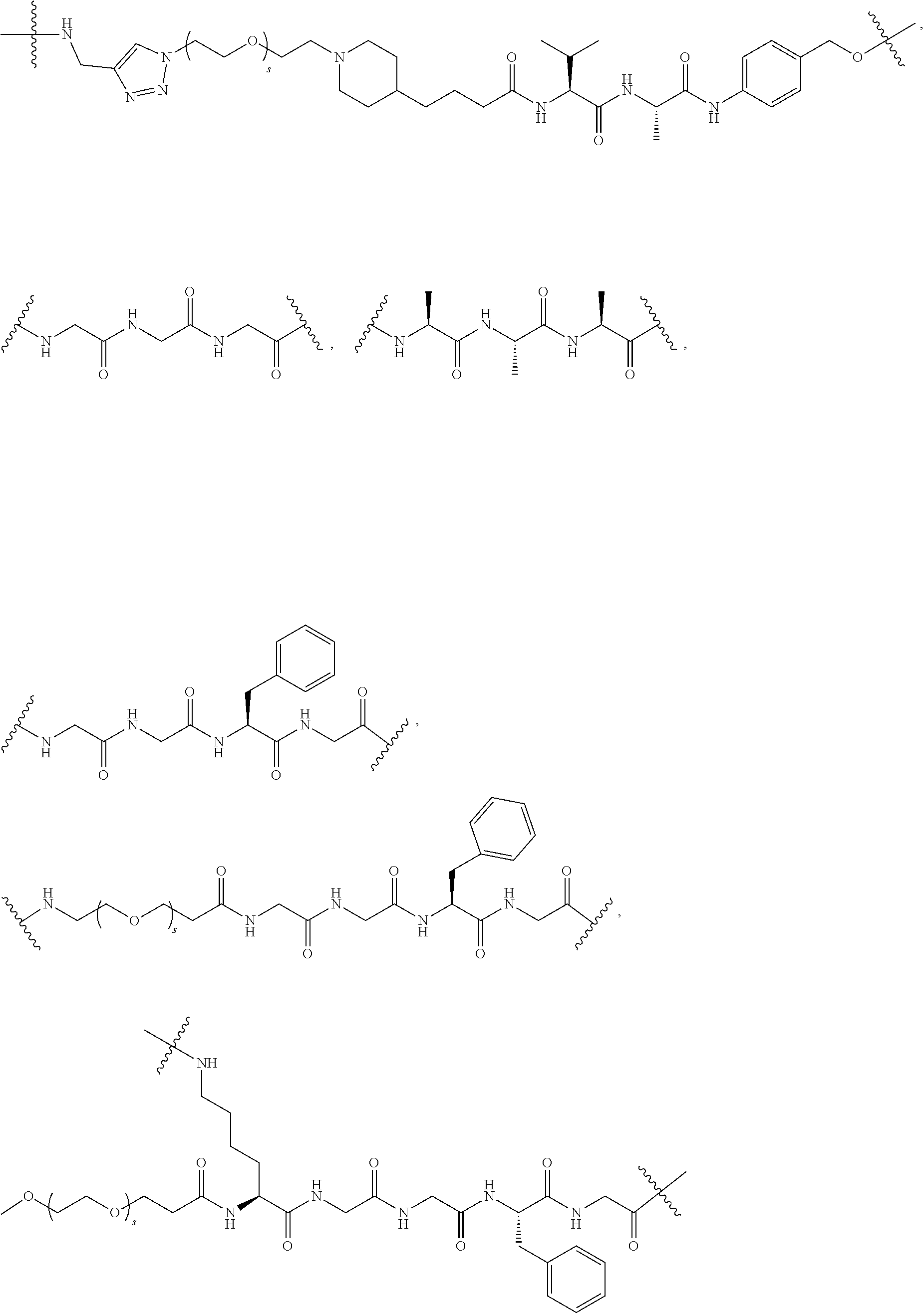

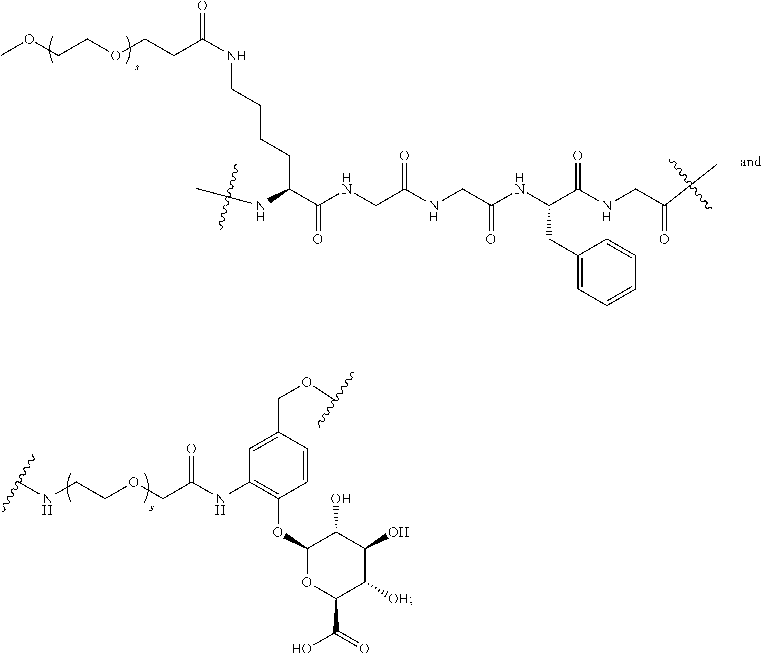

where s is selected from an integer from 1-20.

In some embodiments, L is selected from the following structures:

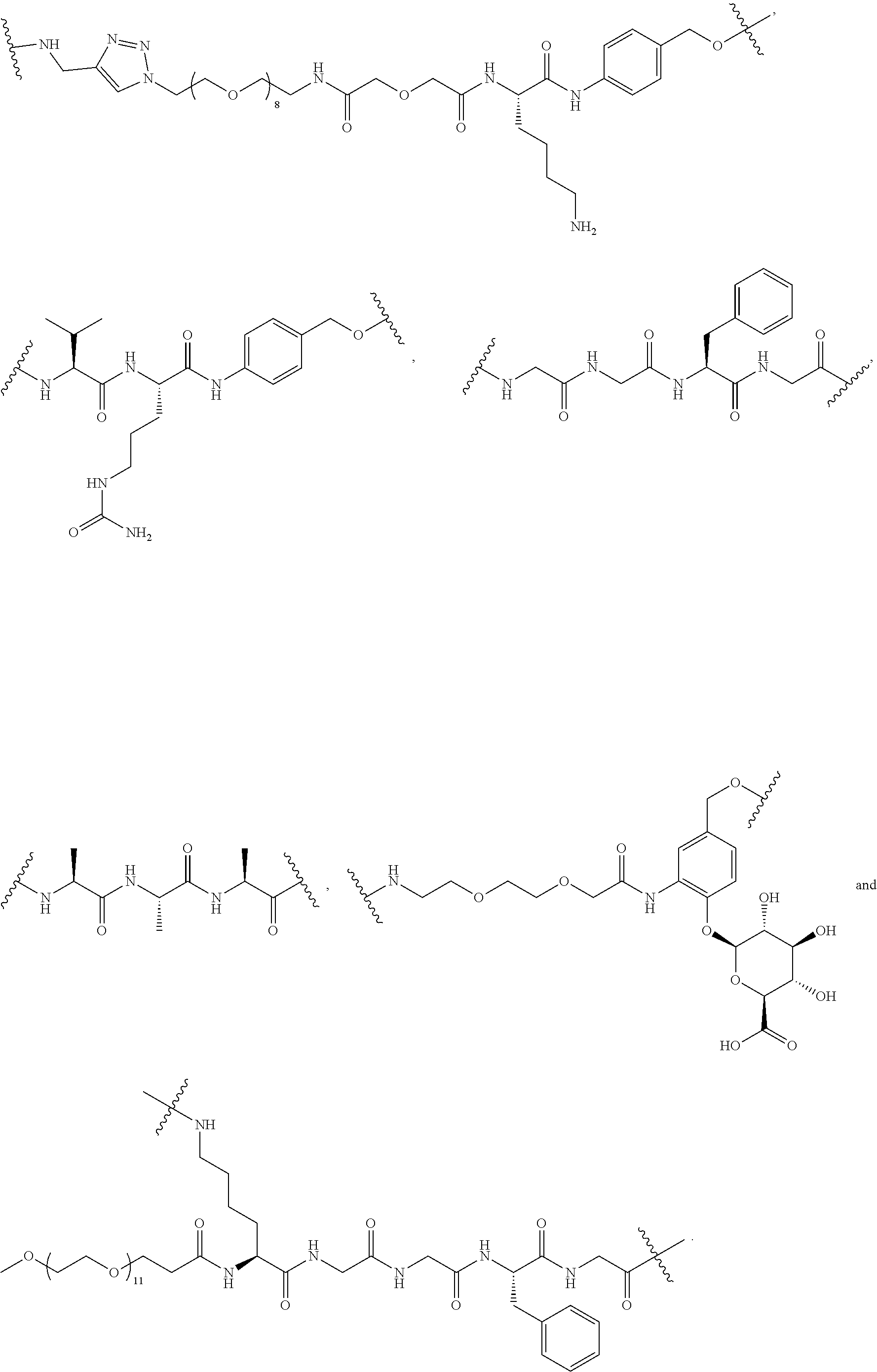

In some embodiments, L is selected from the following structures:

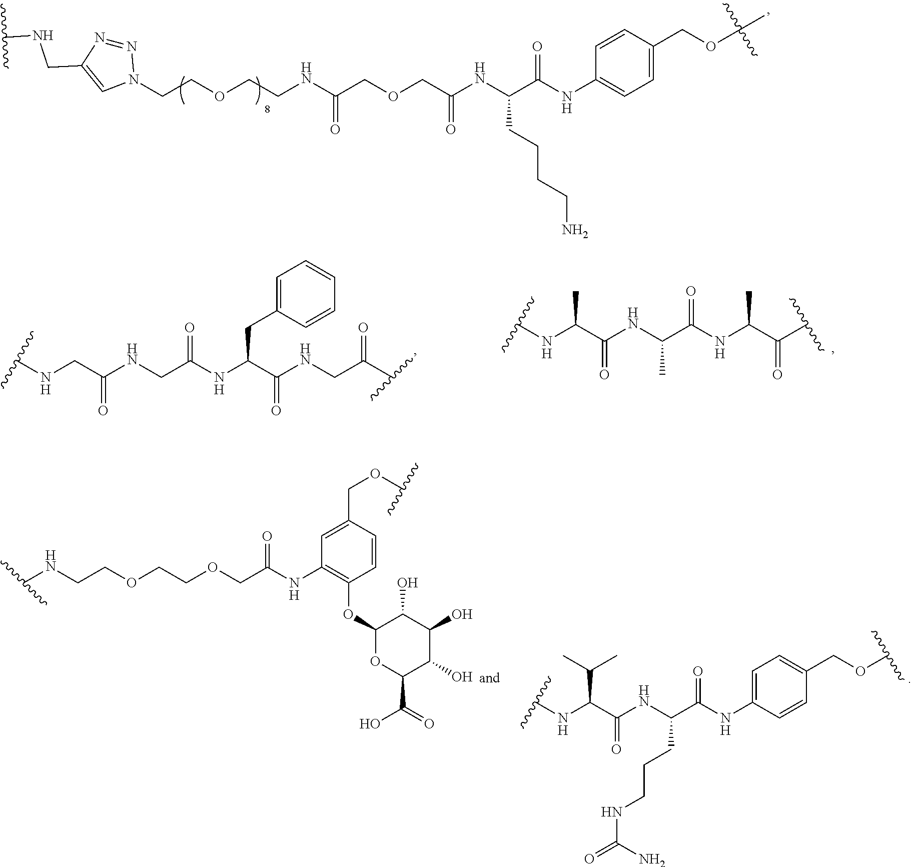

In some embodiments, L is selected from the following structures:

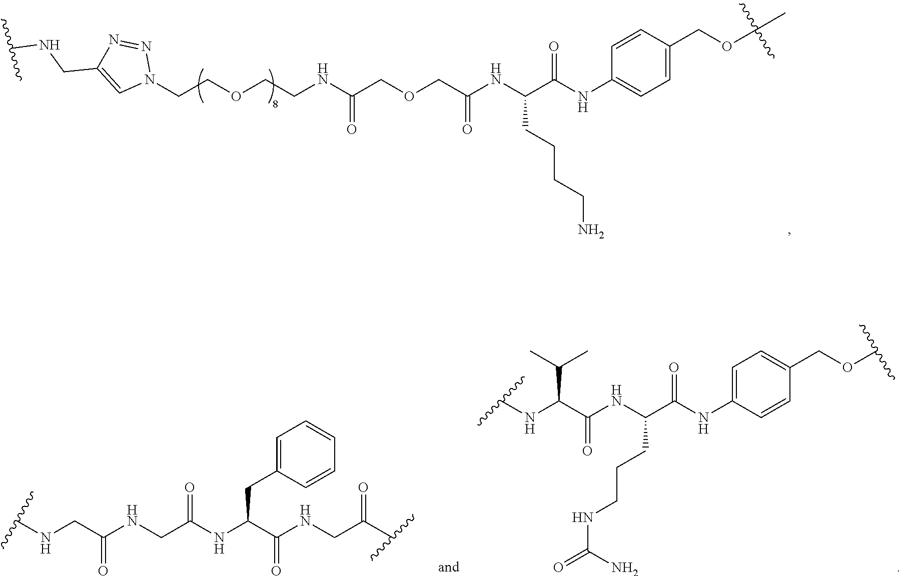

In some embodiments, E is a single bond, —NH—CH₂—, —NH—CH₂—O—CH₂—CO—,

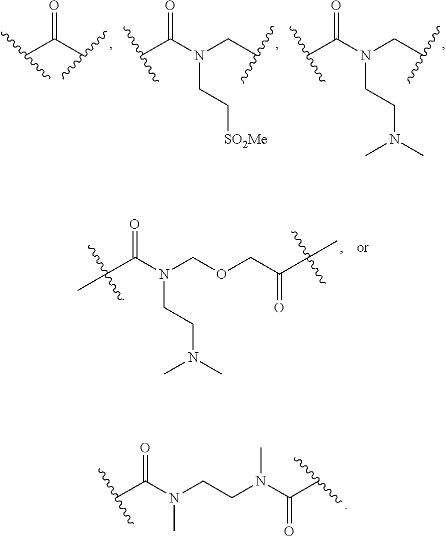

In some embodiments, E is a single bond, —NH—CH₂—, —NH—CH₂—O—CH₂—CO—,

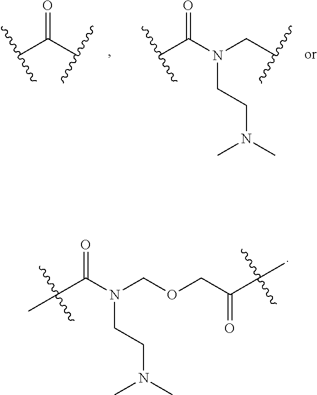

In some embodiments, E is —NH—CH₂—,

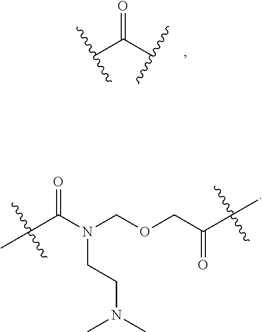

In some embodiments, E is —NH—CH₂— or

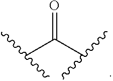 [SMILES: *C(*)=O]

In some embodiments, M is selected from the following structures:

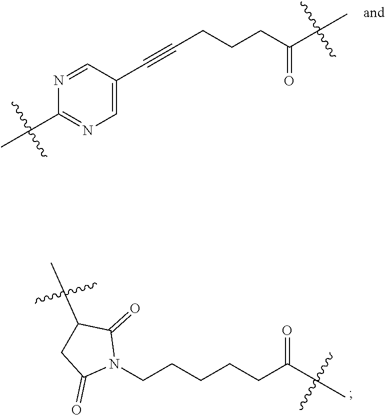 [SMILES: *C(=O)CCCC#Cc1cnc(*)nc1 ; *C(=O)CCCCCN1C(=O)CC(*)C1=O]

L is selected from the following structures:

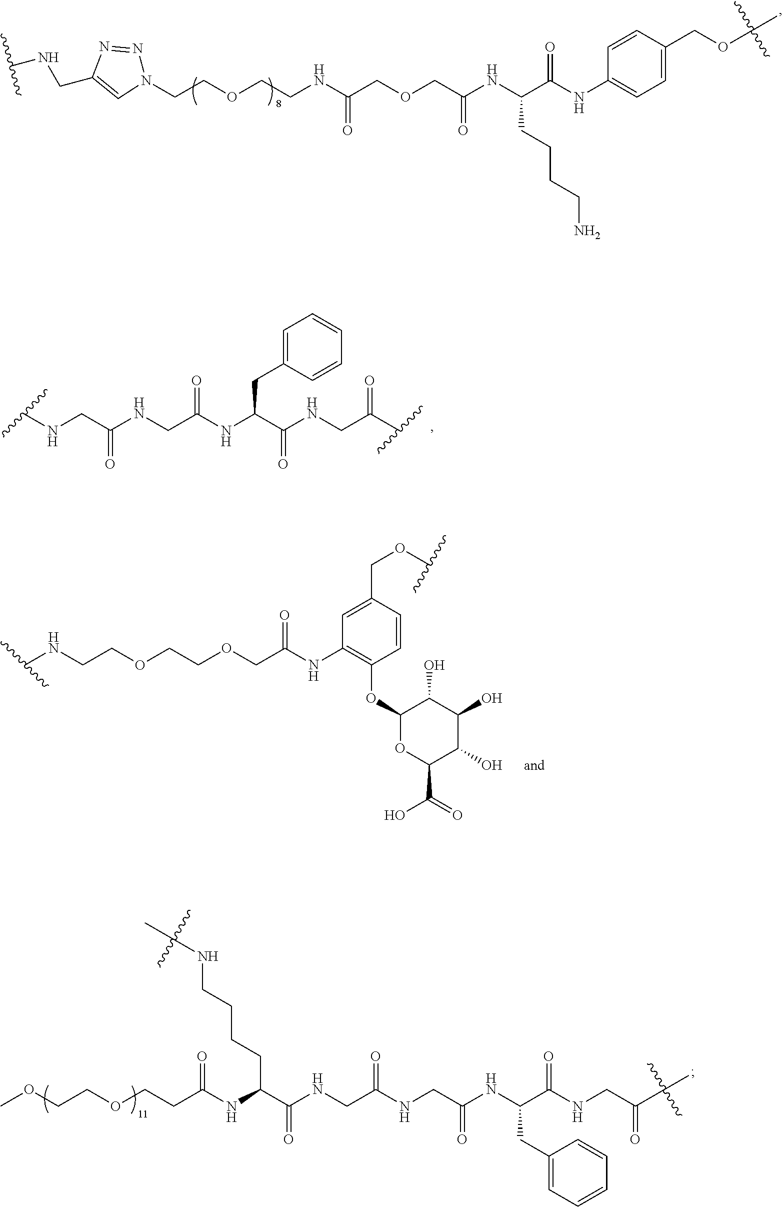

E is —NH—CH₂,

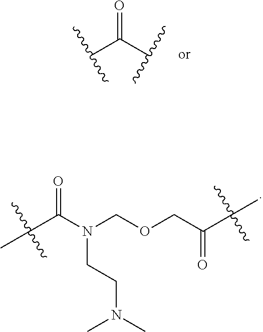

In some embodiments,

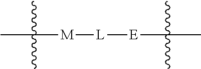

is selected from the following structures:

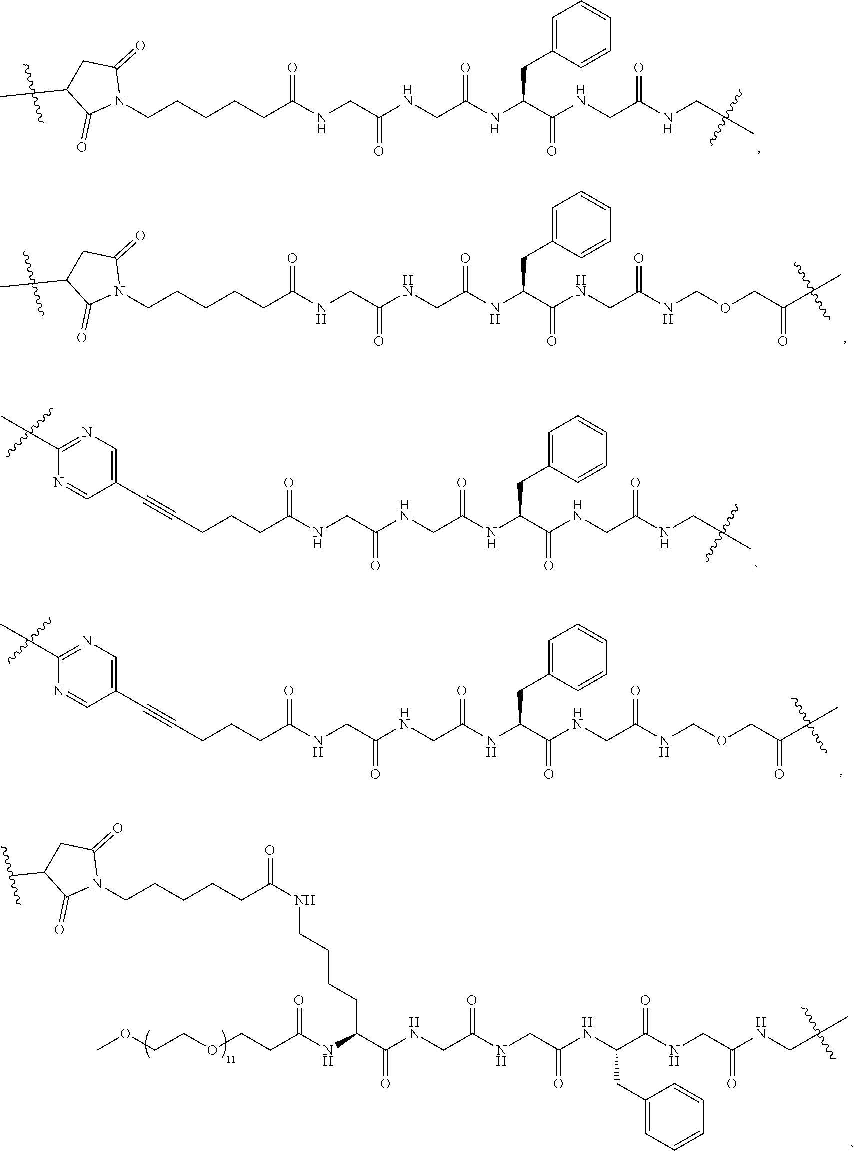

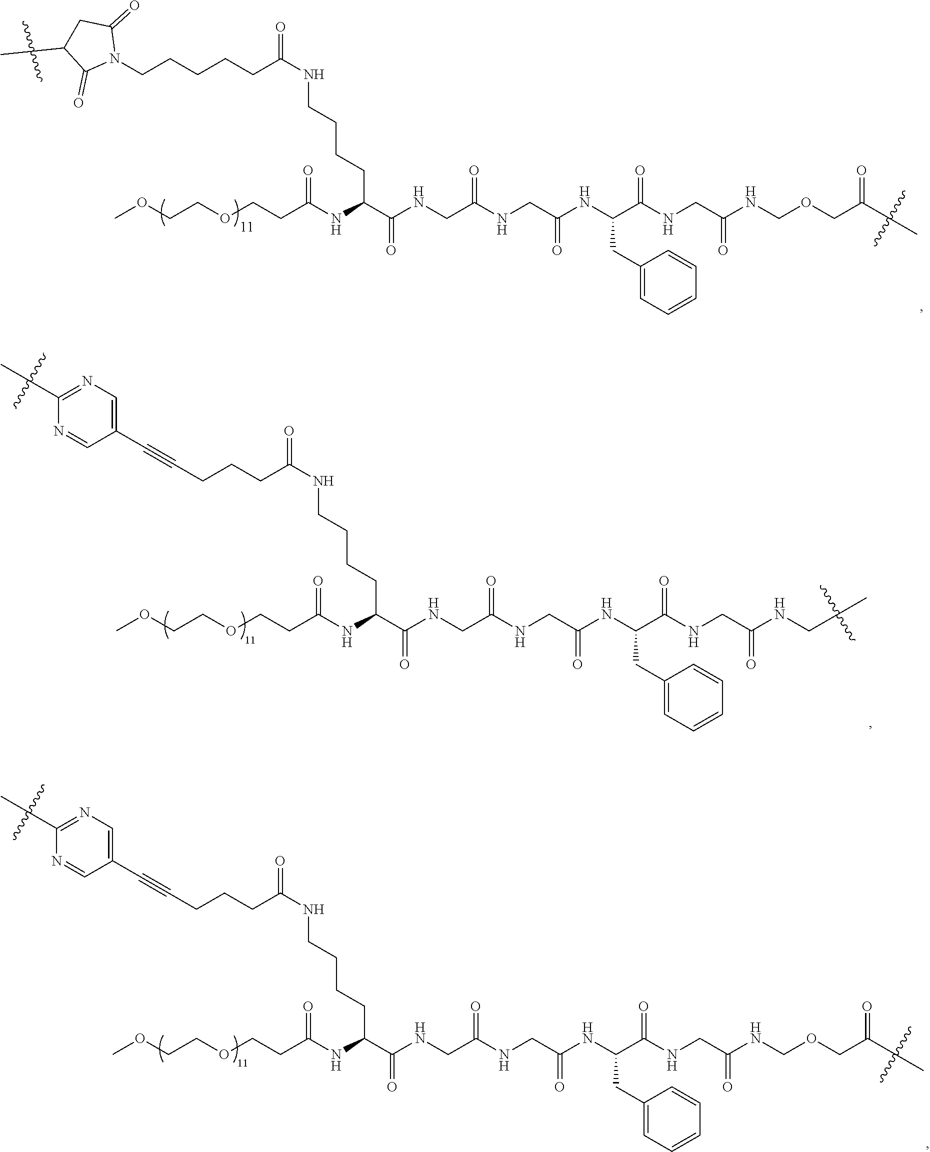

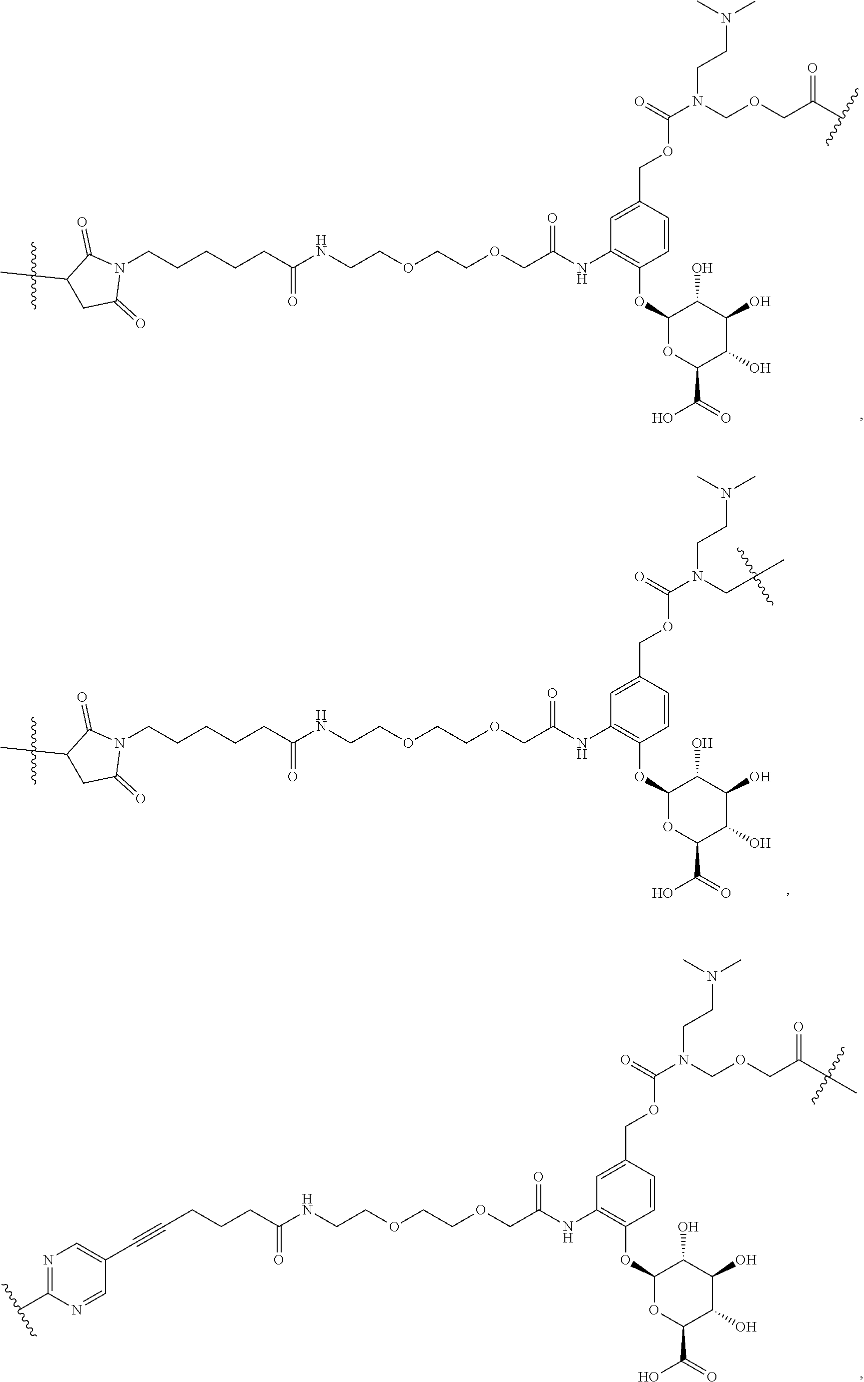 [SMILES: *C(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1 ; *CN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1 ; *C(=O)COCN(CCN(C)C)C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1]

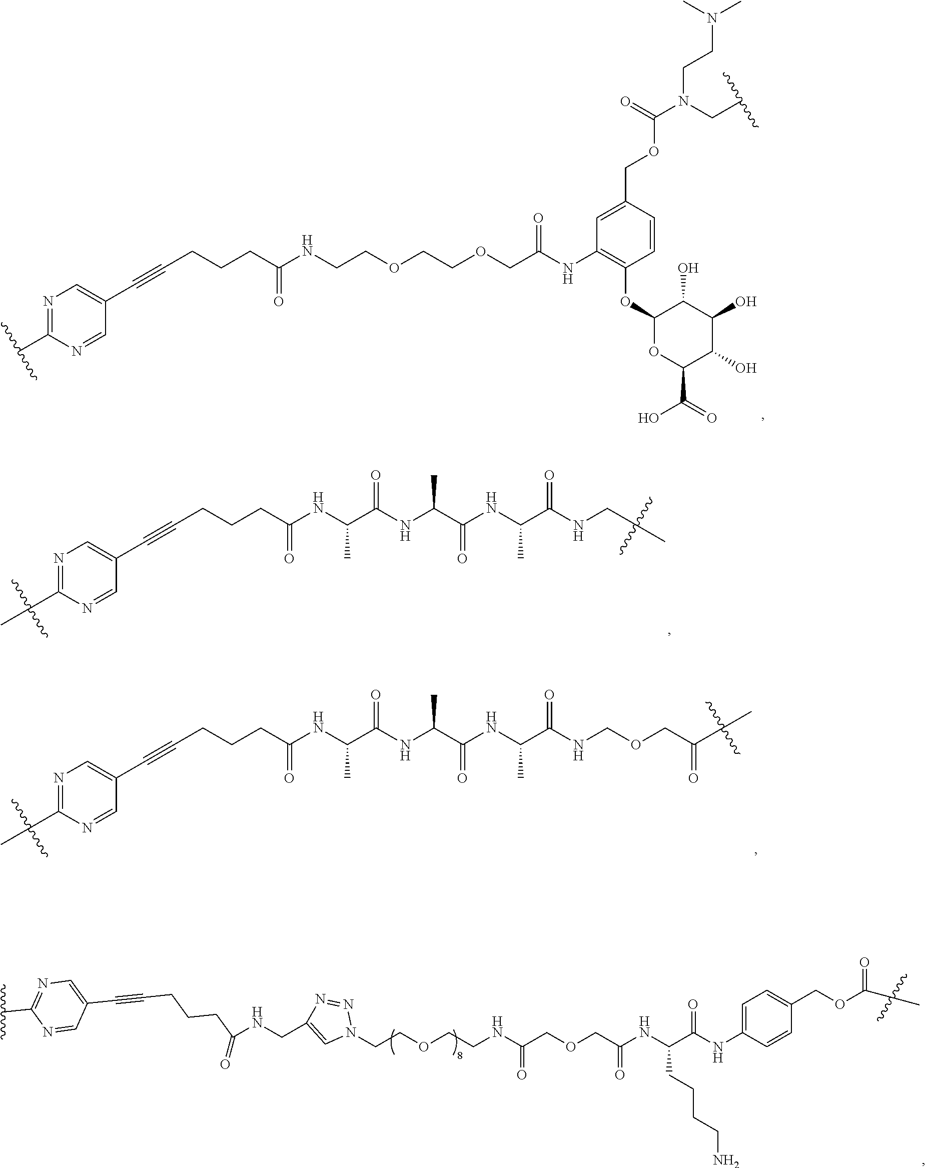

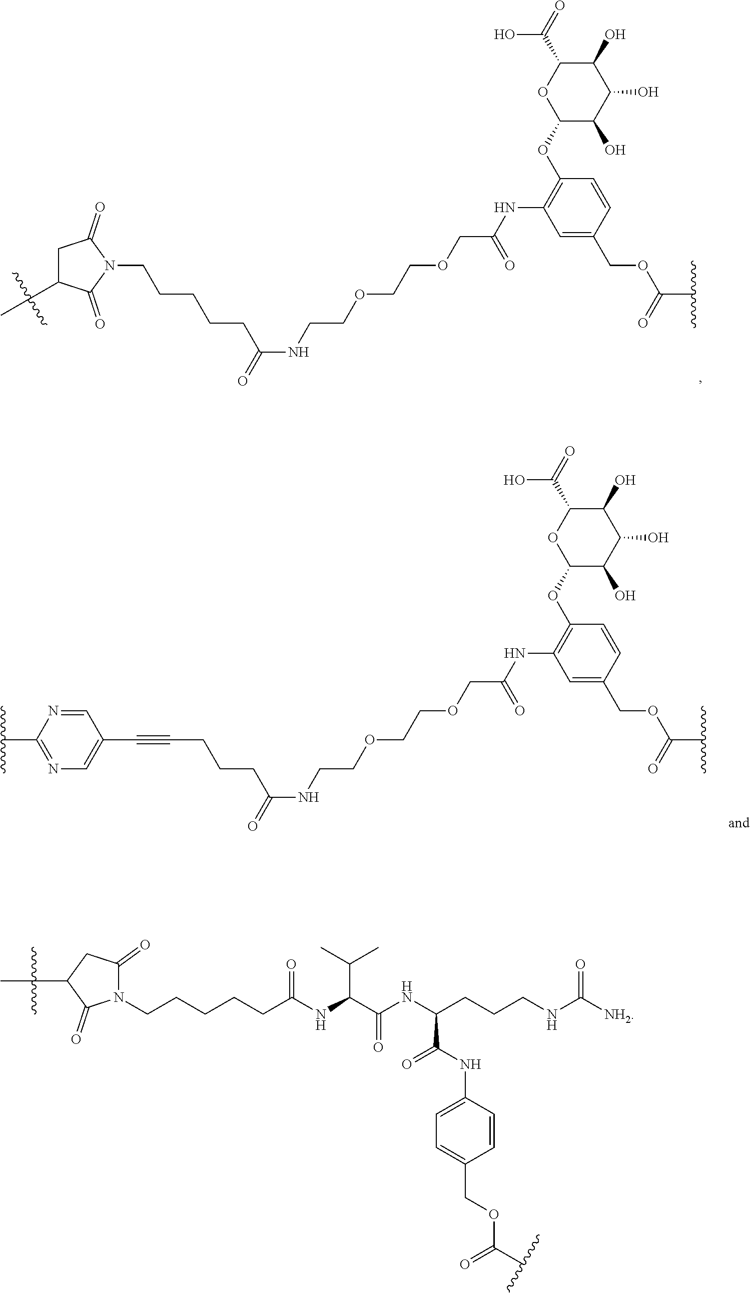 [SMILES: *C(=O)OCc1ccc(O[C@@H]2O[C@H](C(=O)O)[C@@H](O)[C@H](O)[C@H]2O)c(NC(=O)COCCOCCNC(=O)CCCCCN2C(=O)CC(*)C2=O)c1 ; *C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1 ; *C(=O)OCc1ccc(NC(=O)[C@H](CCCNC(N)=O)NC(=O)[C@@H](NC(=O)CCCCCN2C(=O)CC(*)C2=O)C(C)C)cc1]

In some embodiments,

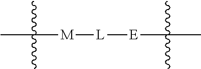

is selected from the following structures:

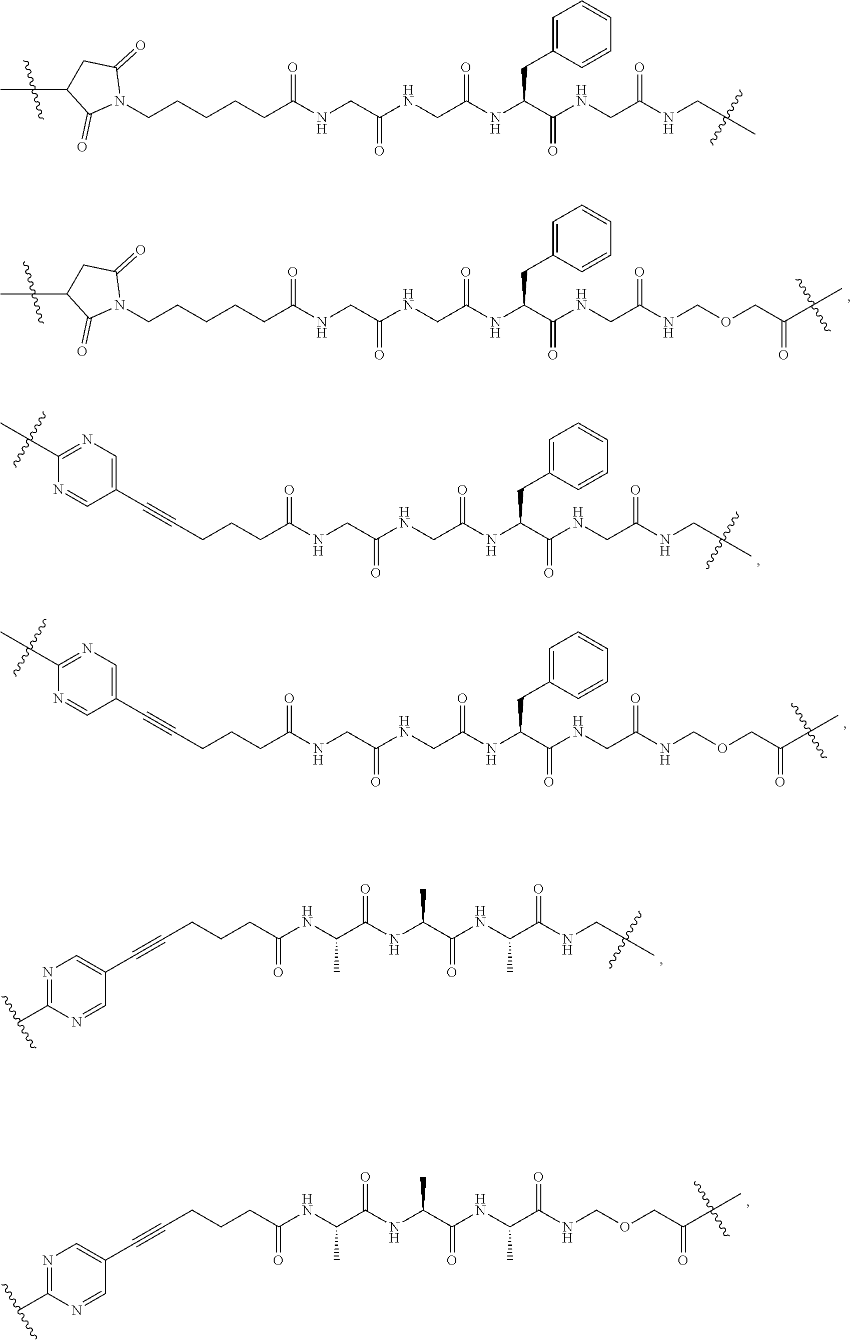 [SMILES: *CNC(=O)CNC(=O)[C@H](Cc1ccccc1)NC(=O)CNC(=O)CNC(=O)CCCCCN1C(=O)CC(*)C1=O ; *C(=O)COCNC(=O)CNC(=O)[C@H](Cc1ccccc1)NC(=O)CNC(=O)CNC(=O)CCCCCN1C(=O)CC(*)C1=O ; *CNC(=O)CNC(=O)[C@H](Cc1ccccc1)NC(=O)CNC(=O)CNC(=O)CCCC#Cc1cnc(*)nc1 ; *C(=O)COCNC(=O)CNC(=O)[C@H](Cc1ccccc1)NC(=O)CNC(=O)CNC(=O)CCCC#Cc1cnc(*)nc1 ; *CNC(=O)[C@H](C)NC(=O)[C@H](C)NC(=O)[C@H](C)NC(=O)CCCC#Cc1cnc(*)nc1 ; *C(=O)COCNC(=O)[C@H](C)NC(=O)[C@H](C)NC(=O)[C@H](C)NC(=O)CCCC#Cc1cnc(*)nc1]

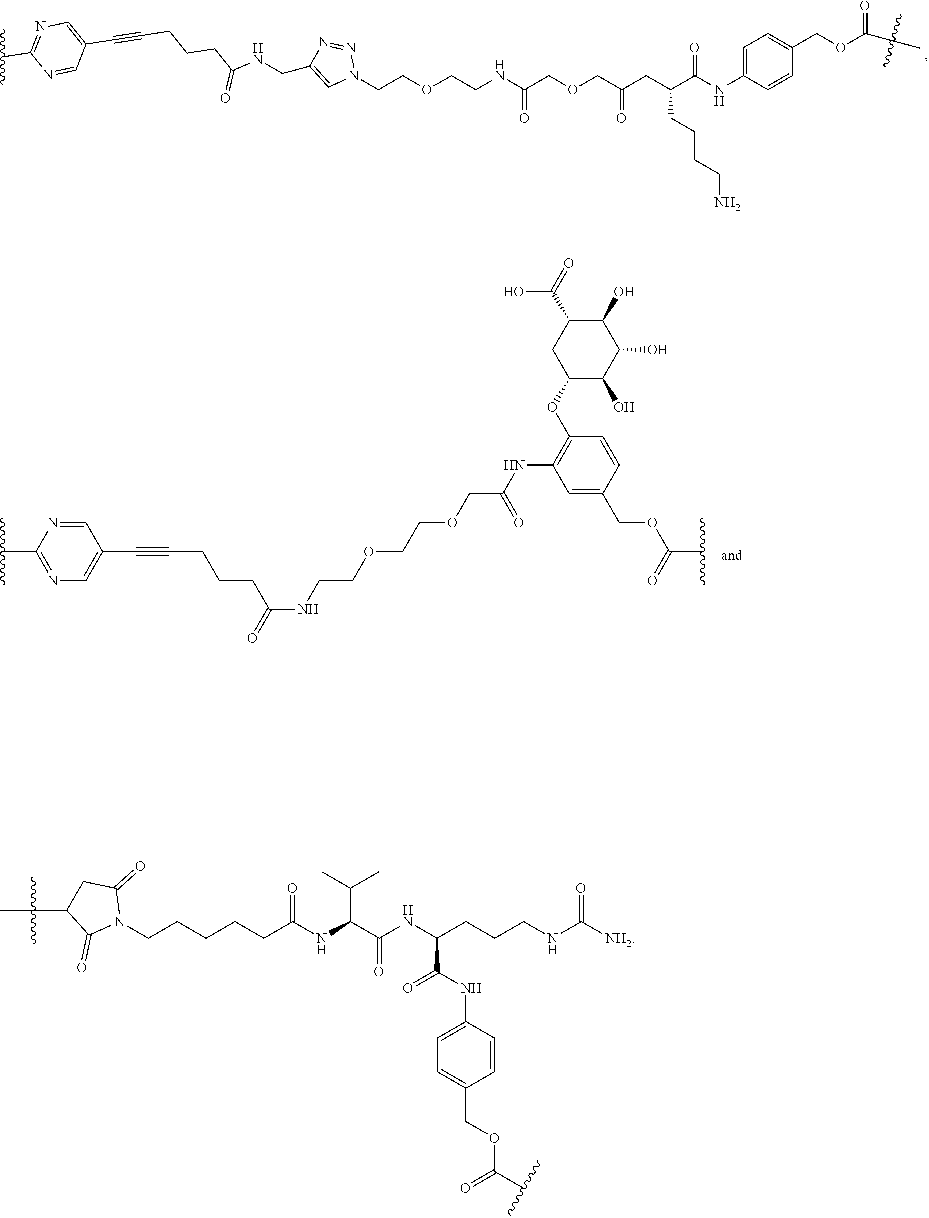 [SMILES: *C(=O)OCc1ccc(NC(=O)[C@H](CCCCN)CC(=O)COCC(=O)NCCOCCn2cc(CNC(=O)CCCC#Cc3cnc(*)nc3)nn2)cc1 ; *C(=O)OCc1ccc(O[C@@H]2C[C@H](C(=O)O)[C@@H](O)[C@H](O)[C@H]2O)c(NC(=O)COCCOCCNC(=O)CCCC#Cc2cnc(*)nc2)c1 ; *C(=O)OCc1ccc(NC(=O)[C@H](CCCNC(N)=O)NC(=O)[C@@H](NC(=O)CCCCCN2C(=O)CC(*)C2=O)C(C)C)cc1]

In some embodiments,

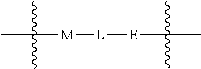

is selected from the following structures:

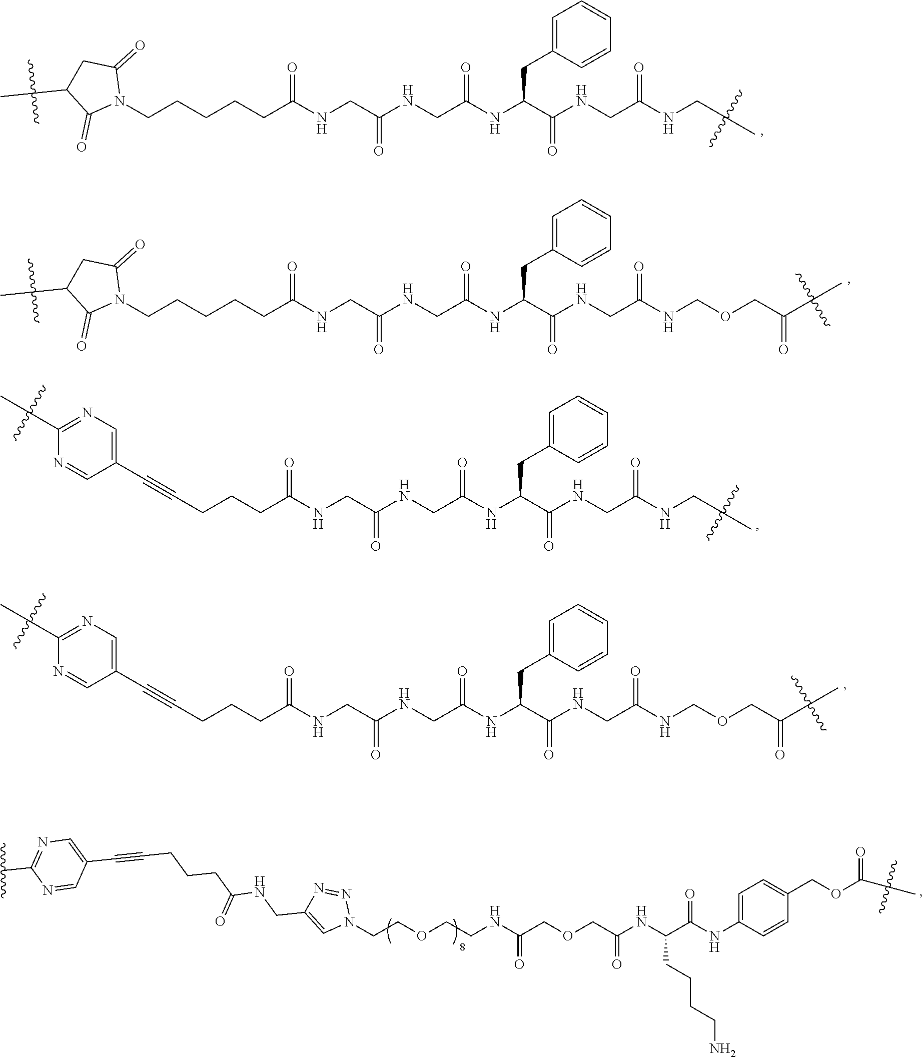

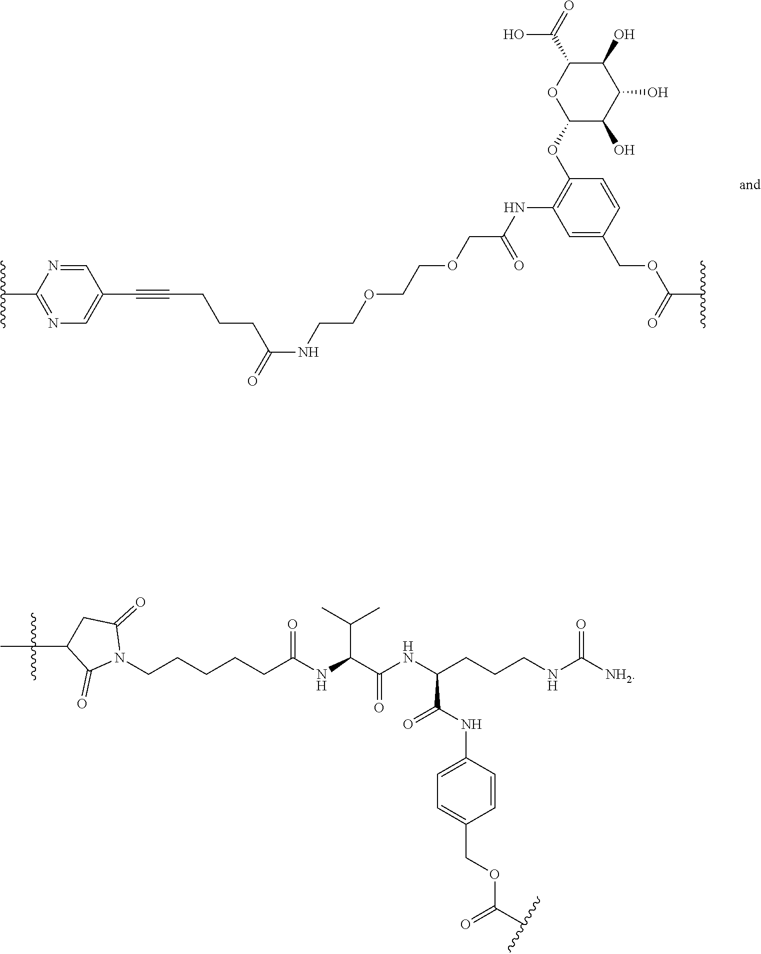 [SMILES: *C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1 ; *C(=O)OCc1ccc(NC(=O)[C@H](CCCNC(N)=O)NC(=O)[C@@H](NC(=O)CCCCCN2C(=O)CC(*)C2=O)C(C)C)cc1]

In some embodiments, the cytotoxic drug is selected from a tubulin inhibitor, a DNA intercalator, a DNA topoisomerase inhibitor and a RNA polymerase inhibitor. In some embodiments, the tubulin inhibitor is an auristatin compound or a maytansinoid compound. In some embodiments, the DNA intercalator is pyrrolobenzodiazepine (PBD). In some embodiments, the DNA topoisomerase inhibitor is a topoisomerase I inhibitor (for example, camptothecin, hydroxycamptothecin, 9-aminocamptothecin, SN-38, irinotecan, topotecan, belotecan, or rubitecan) or a topoisomerase II inhibitor (for example, doxorubicin, PNU-159682, duocarmycin, daunorubicin, mitoxantrone, podophyllotoxin, or etoposide). In some embodiments, the RNA polymerase inhibitor is a-amanitin or a pharmaceutically acceptable salt, ester or analog thereof.

The cytotoxic drug disclosed in the present application usually contains a variety of functional groups, for example hydroxyl (—OH), carboxyl (—COOH), sulfhydryl (—SH), primary amine (—NH₂), secondary amine (—NR_{A}H) or a tertiary amine group (—NR_{B}R_{C}), and R_{A}, R_{B} and R_{C} herein only represent non-hydrogen substitutes on N; and the cytotoxic drug can be linked to the linker in the conjugate through these functional groups.

In some embodiments, the cytotoxic drug is linked to E in the antibody-drug conjugate through —OH, —SH, primary amine group, secondary amine group or tertiary amine group thereon.

In some embodiments, the cytotoxic drug is

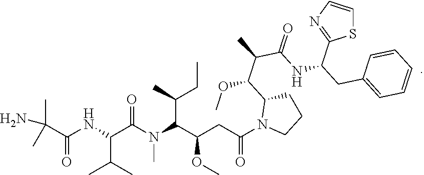

In some embodiments, the cytotoxic drug is selected from the following formulas I and II:

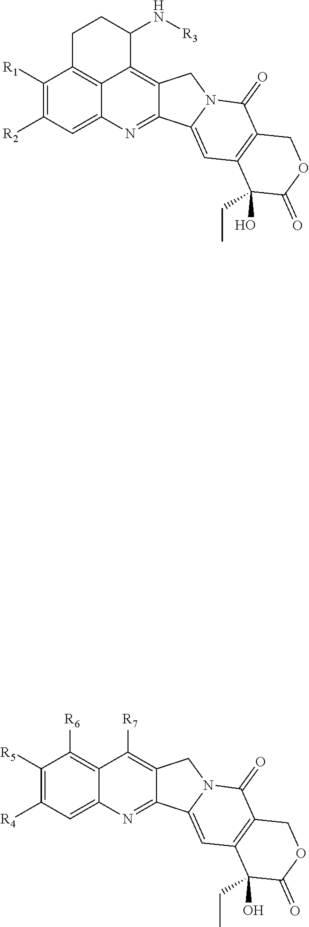

wherein
R₁ and R₂ are each independently selected from C₁₋₆ alkyl and halogen;
R₃ is selected from H and —CO—CH₂OH;
R₄ and R₅ are each independently selected from H, halogen and hydroxyl; or R₄ and R₅ are linked to associated carbon atoms to form 5-6 membered oxygen-containing heterocyclic ring;
R₆ is selected from hydrogen and —C₁₋₄ alkylene-NRₐR_{b};
R₇ is selected from hydrogen, C₁₋₆ alkyl and —C₁₋₄ alkylene-NRₐR_{b}; and wherein Rₐ and R_{b} are each independently selected from H, C₁₋₆ alkyl, —SO₂-C₁₋₆ alkyl and —CO—C₁₋₆ alkyl at each occurrence.

In some embodiments, the cytotoxic drug is selected from the following compounds:

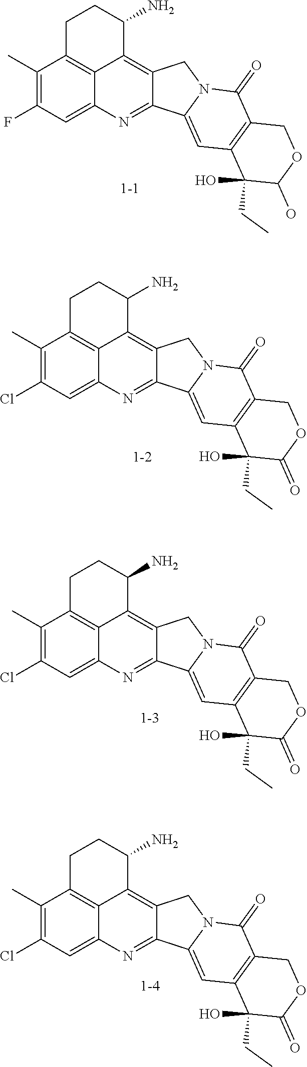

[chemical structure]

In some embodiments, the cytotoxic drug is selected from the following compounds:

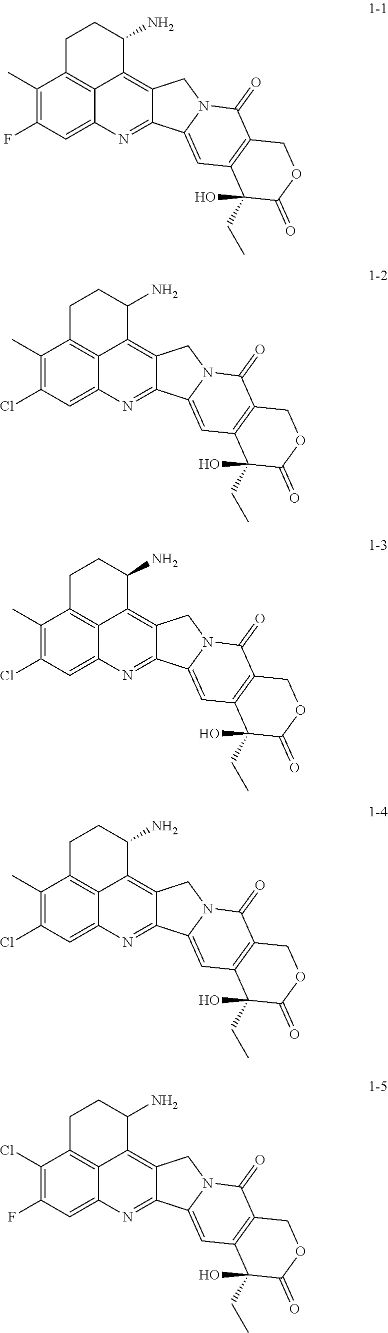 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3]

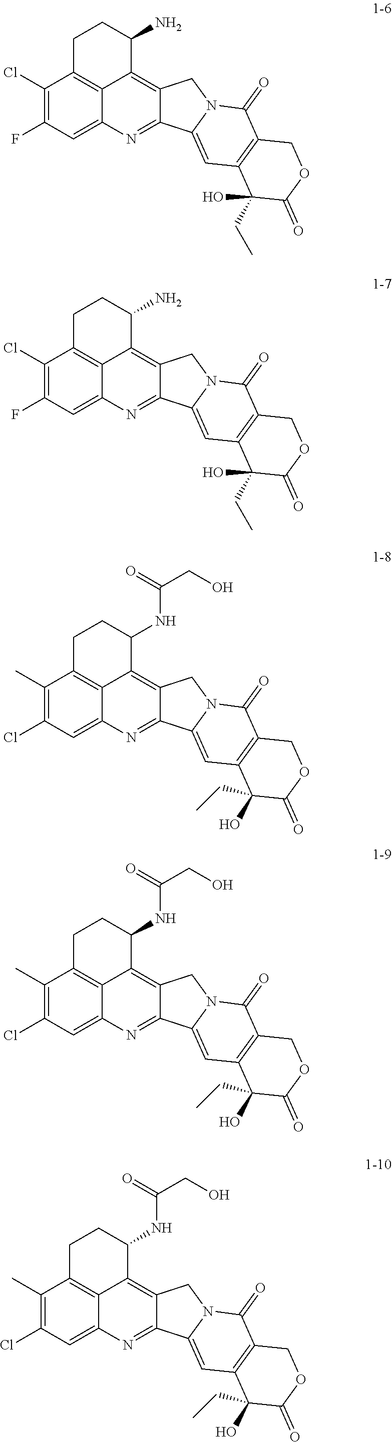 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3]

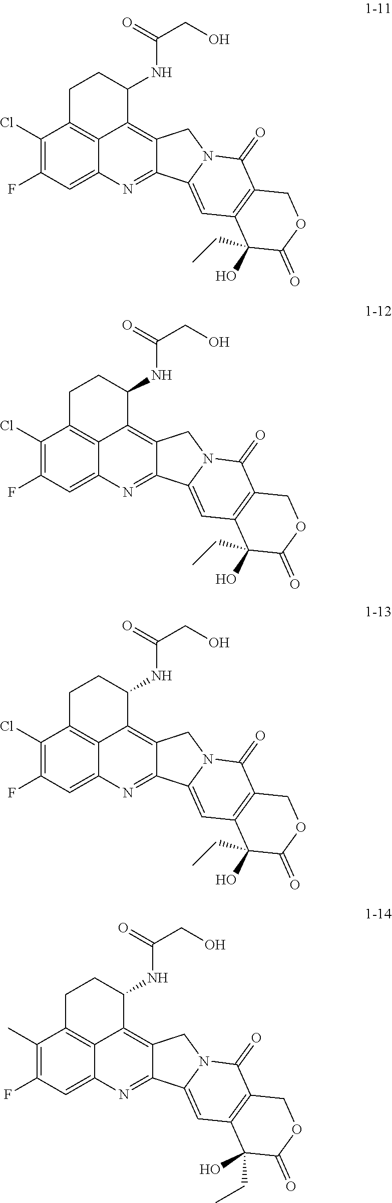 [SMILES: CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3]

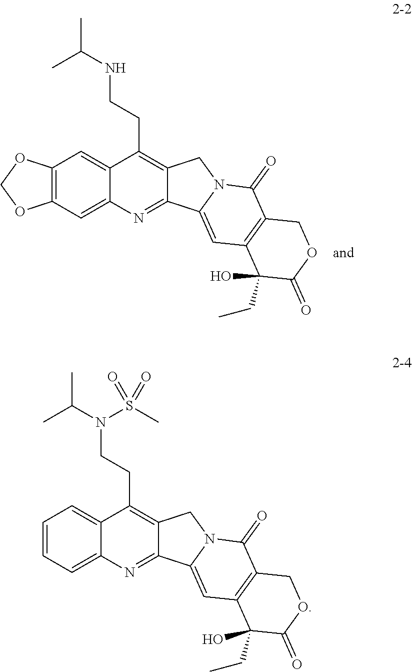 [SMILES: CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1ccccc1c2CCN(C(C)C)S(C)(=O)=O]

The corresponding fragment of the cytotoxic drug obtained after the cytotoxic drug is linked to the linker is D in the formula Ab-[M-L-E-D]ₓ according to the present application. In some embodiments, D is a monovalent structure obtained by the loss of one H from —OH, —NH₂, or secondary amine groups on the cytotoxic drug.

In some embodiments, D is selected from the following structures:

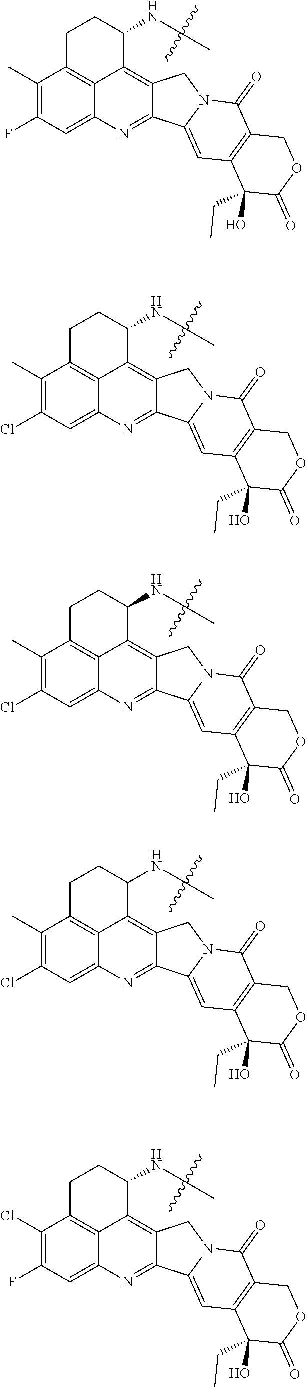 [SMILES: *N[C@H]1CCc2c(C)c(F)cc3nc4c(c1c23)Cn1c-4cc2c(c1=O)COC(=O)[C@]2(O)CC ; *N[C@H]1CCc2c(C)c(Cl)cc3nc4c(c1c23)Cn1c-4cc2c(c1=O)COC(=O)[C@]2(O)CC ; *N[C@@H]1CCc2c(C)c(Cl)cc3nc4c(c1c23)Cn1c-4cc2c(c1=O)COC(=O)[C@]2(O)CC ; *NC1CCc2c(C)c(Cl)cc3nc4c(c1c23)Cn1c-4cc2c(c1=O)COC(=O)[C@]2(O)CC ; *N[C@H]1CCc2c(Cl)c(F)cc3nc4c(c1c23)Cn1c-4cc2c(c1=O)COC(=O)[C@]2(O)CC]

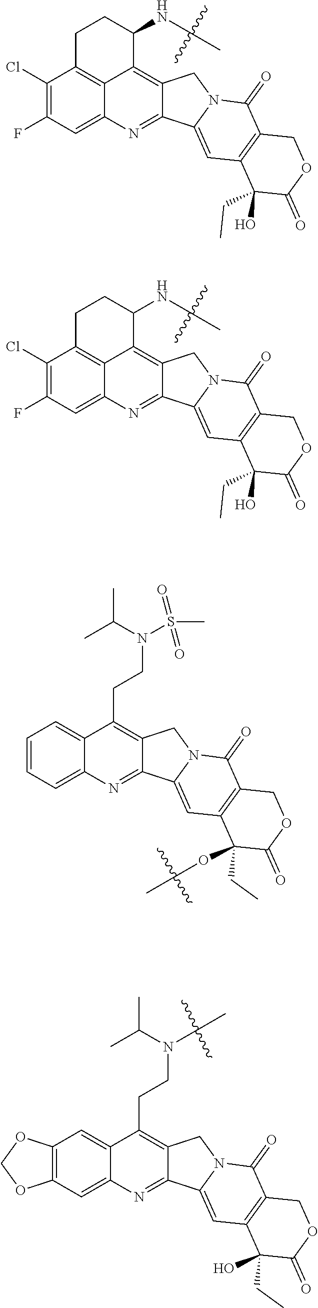 [SMILES: *N[C@@H]1CCc2c(Cl)c(F)cc3nc4c(c1c23)Cn1c-4cc2c(c1=O)COC(=O)[C@]2(O)CC ; *NC1CCc2c(Cl)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1ccccc1c2CCN(C(C)C)S(C)(=O)=O ; *N(CCc1c2c(nc3cc4c(cc13)OCO4)-c1cc3c(c(=O)n1C2)COC(=O)[C@]3(O)CC)C(C)C]

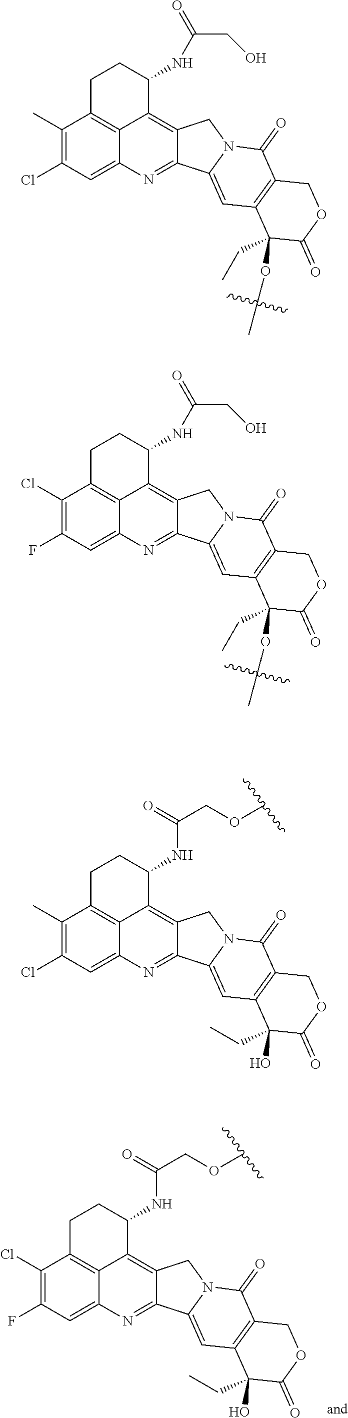 [SMILES: *O[C@]1(CC)C(=O)OCc2c1cc1n(c2=O)Cc2c-1nc1cc(Cl)c(C)c3c1c2[C@@H](NC(=O)CO)CC3 ; *O[C@]1(CC)C(=O)OCc2c1cc1n(c2=O)Cc2c-1nc1cc(F)c(Cl)c3c1c2[C@@H](NC(=O)CO)CC3 ; *OCC(=O)N[C@H]1CCc2c(C)c(Cl)cc3nc4c(c1c23)Cn1c-4cc2c(c1=O)COC(=O)[C@]2(O)CC ; *OCC(=O)N[C@H]1CCc2c(Cl)c(F)cc3nc4c(c1c23)Cn1c-4cc2c(c1=O)COC(=O)[C@]2(O)CC]

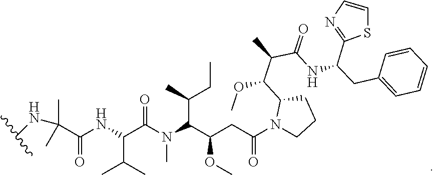

In some embodiments, the antibody-drug conjugate is selected from ADC A-01 to ADC A-26, ADC B-01 to ADC B-06, and ADC C-01 shown as follows:

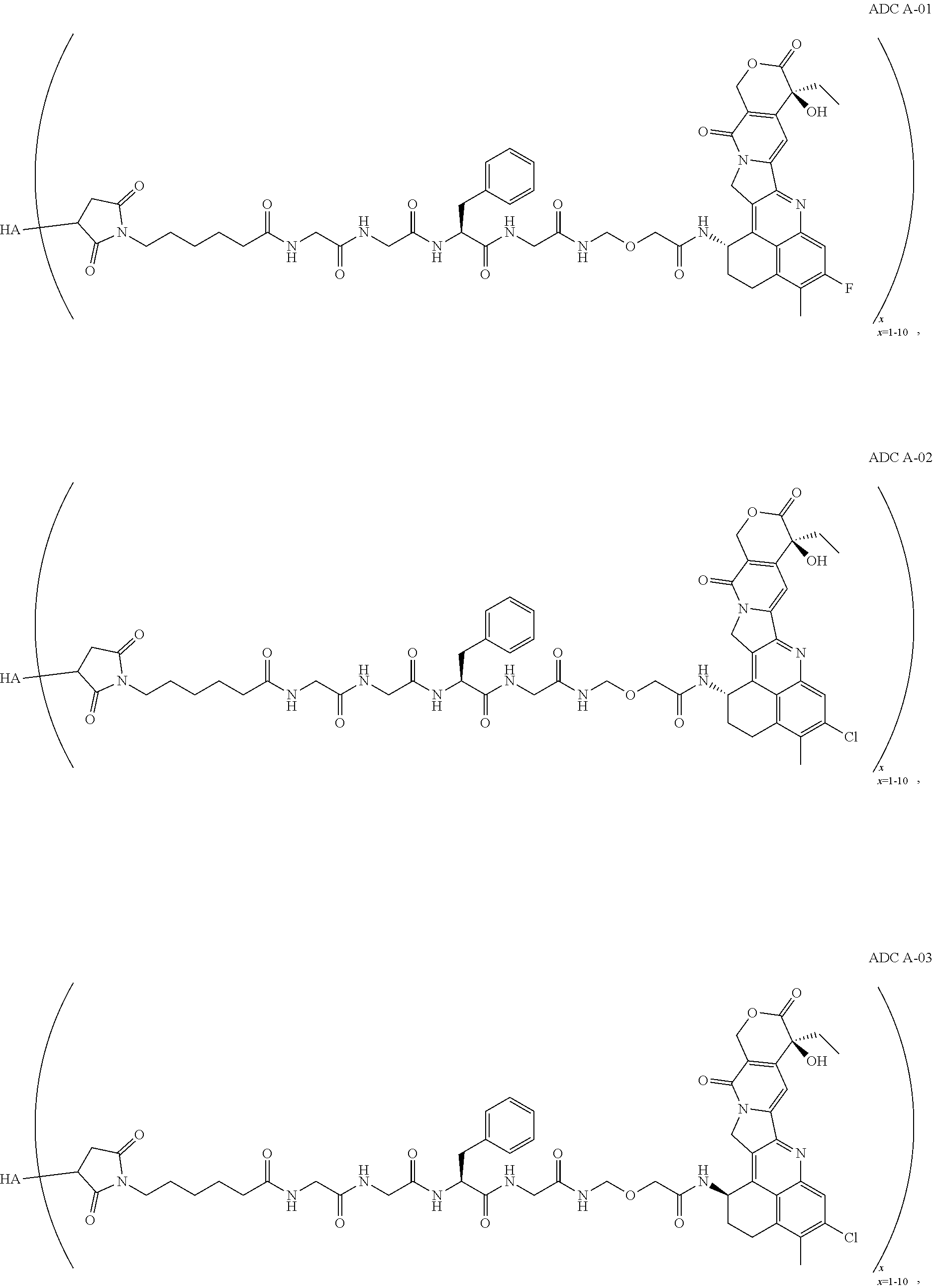

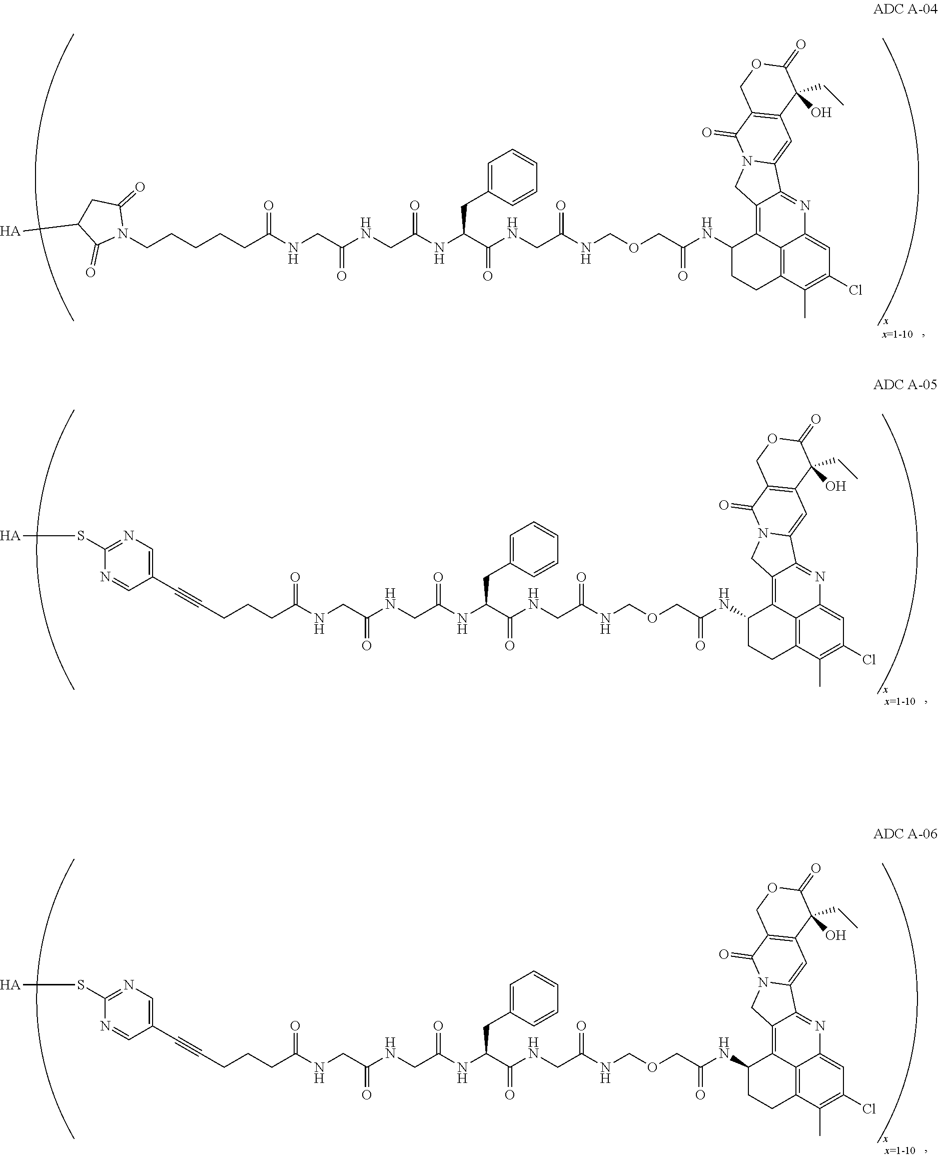

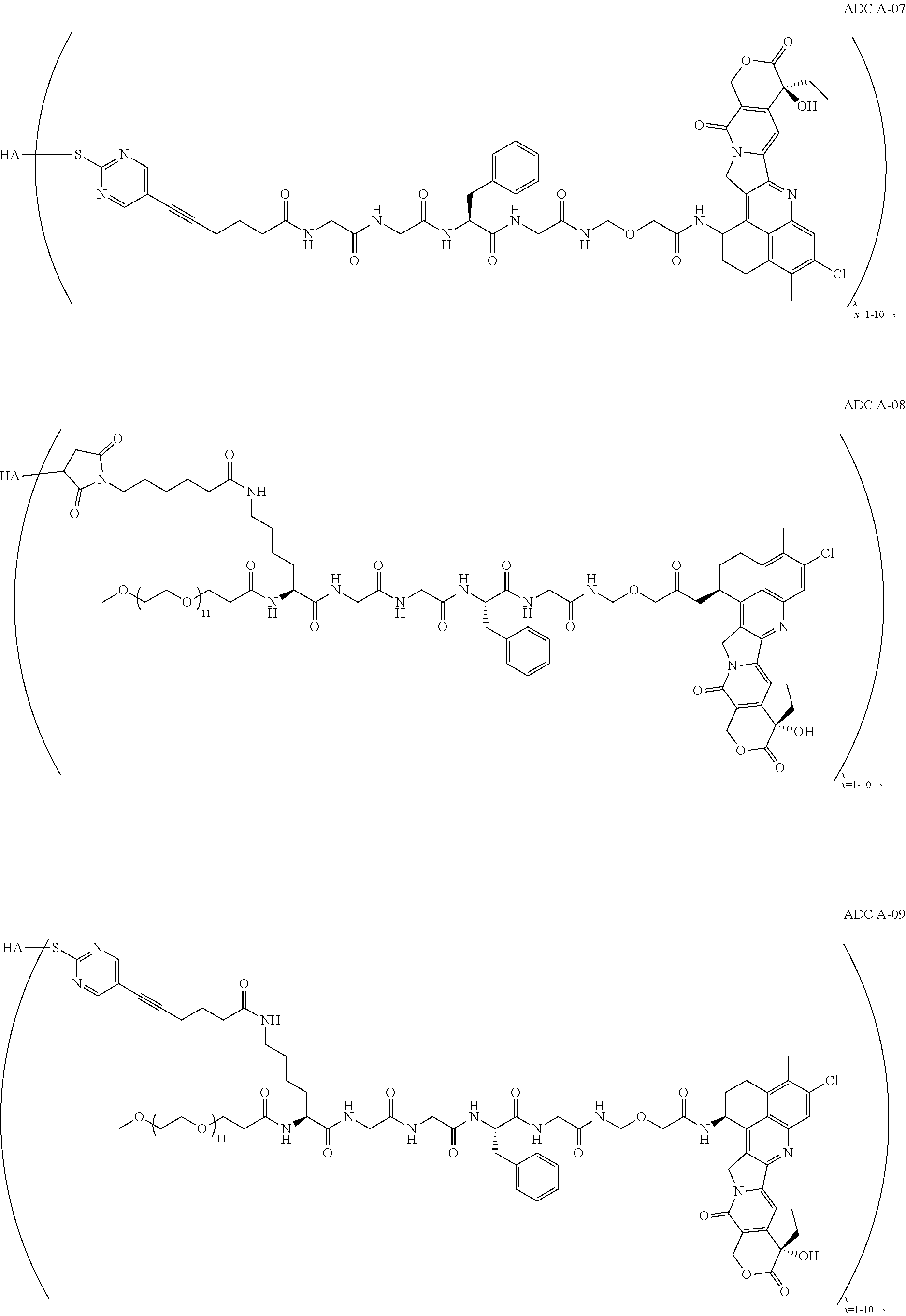

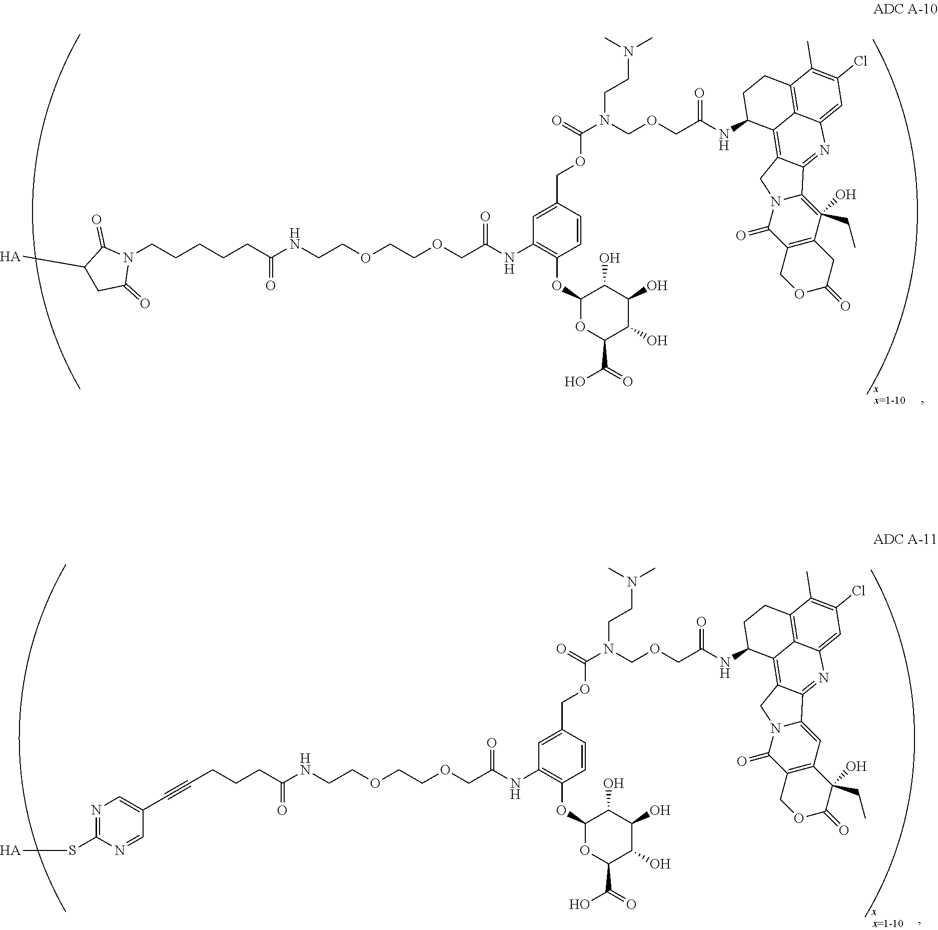

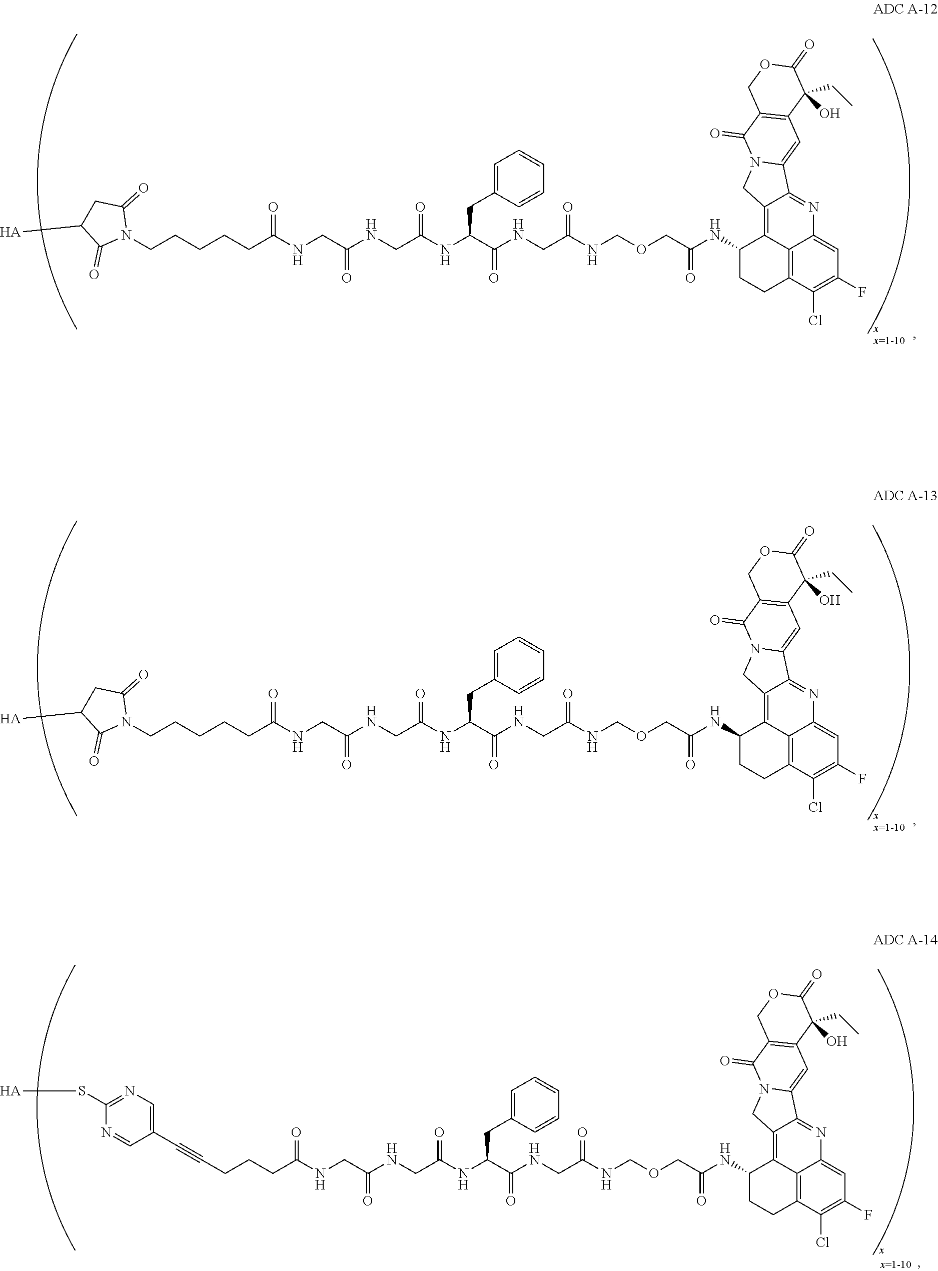

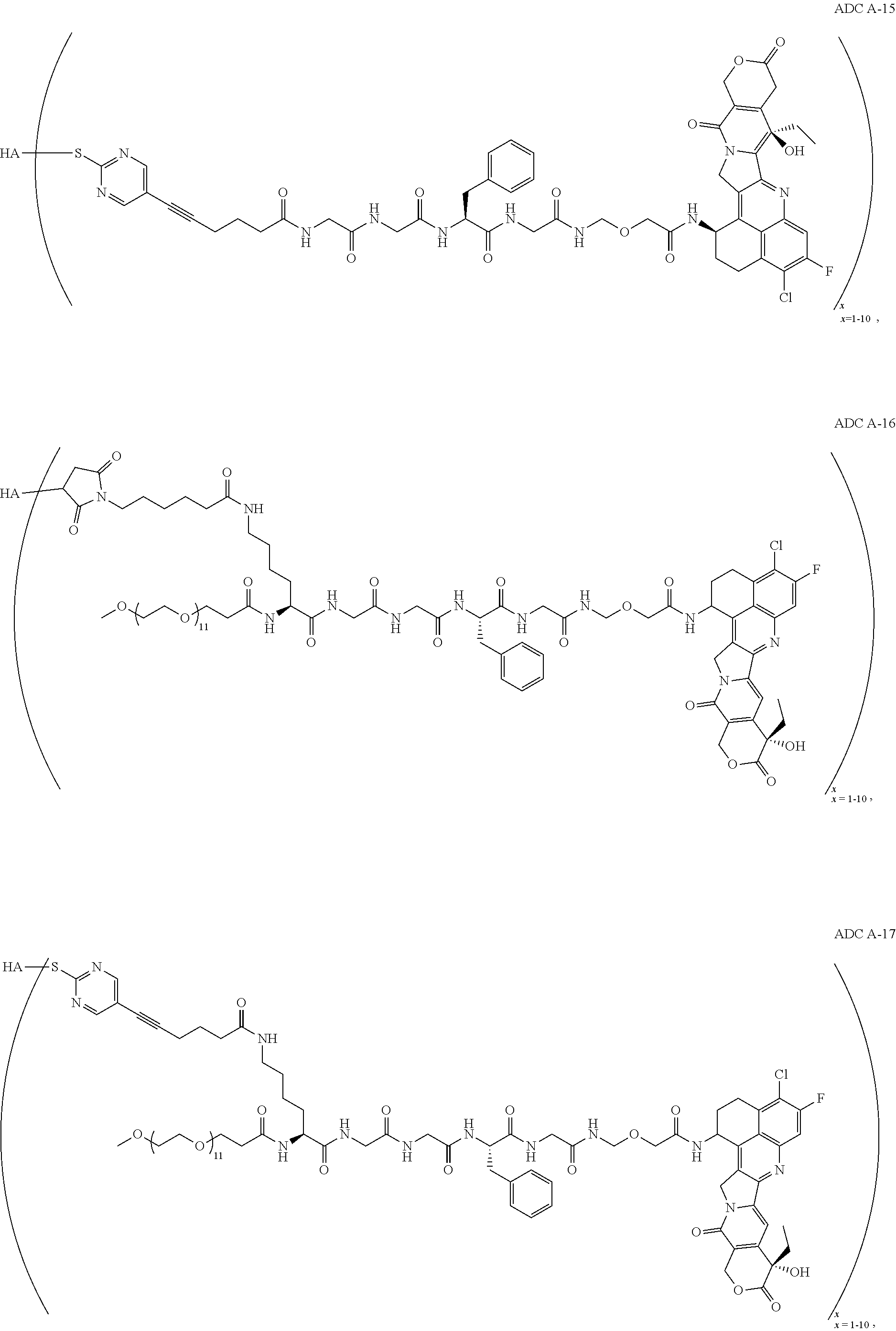

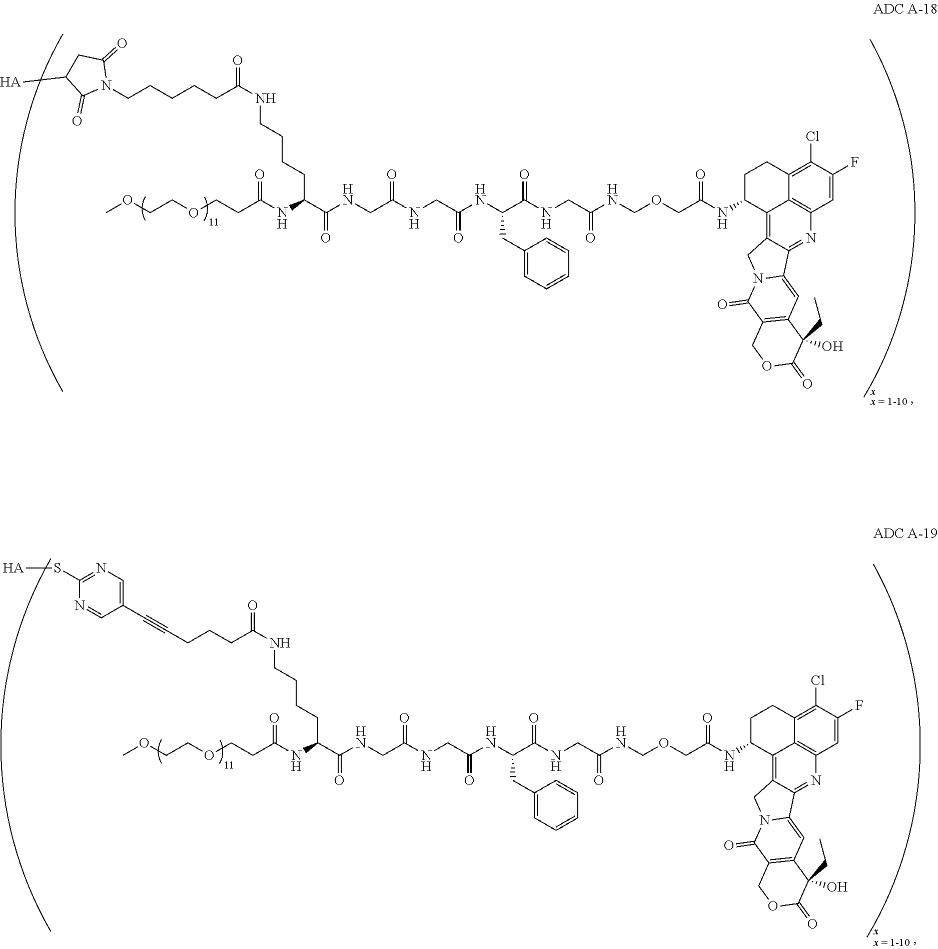

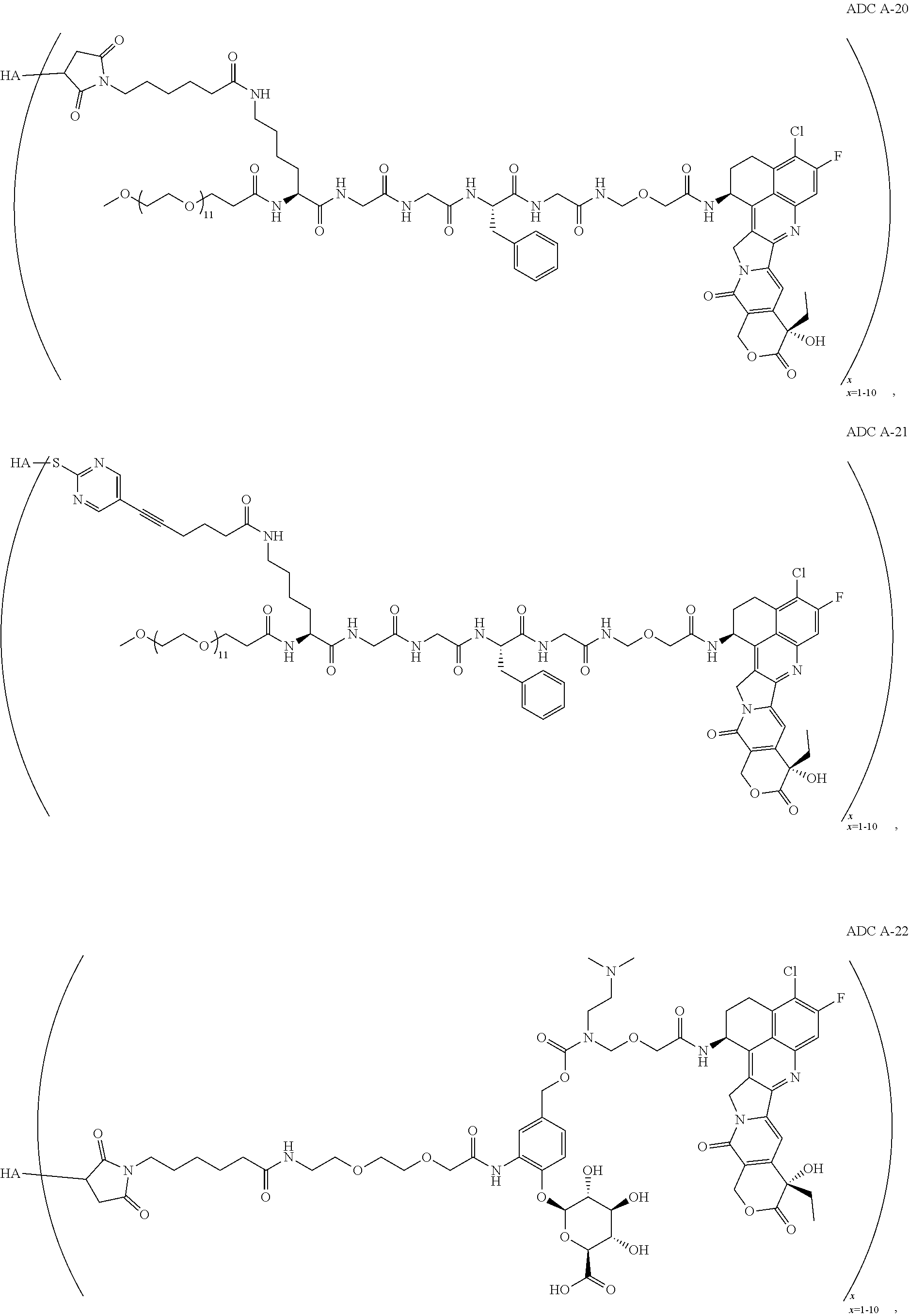

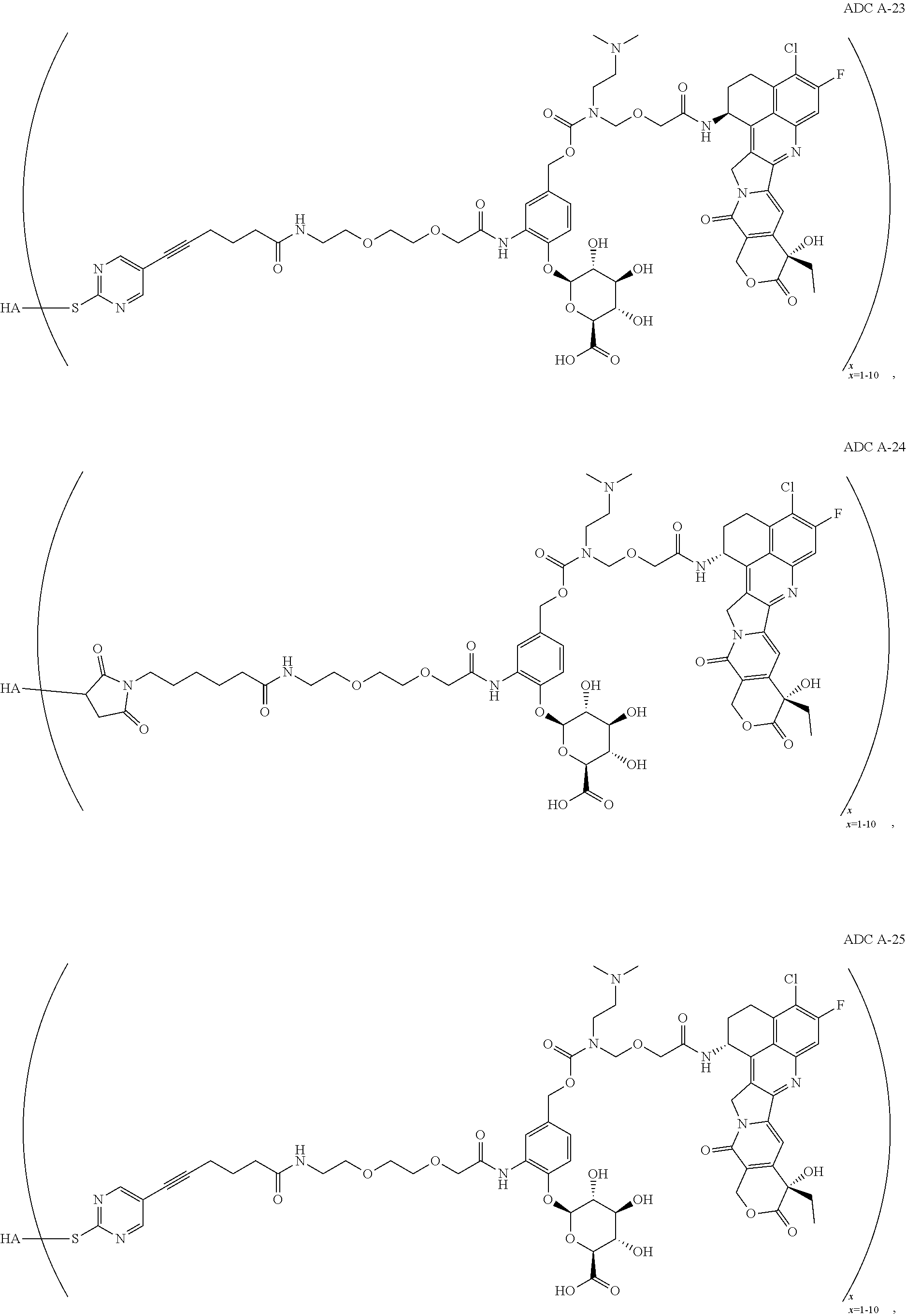

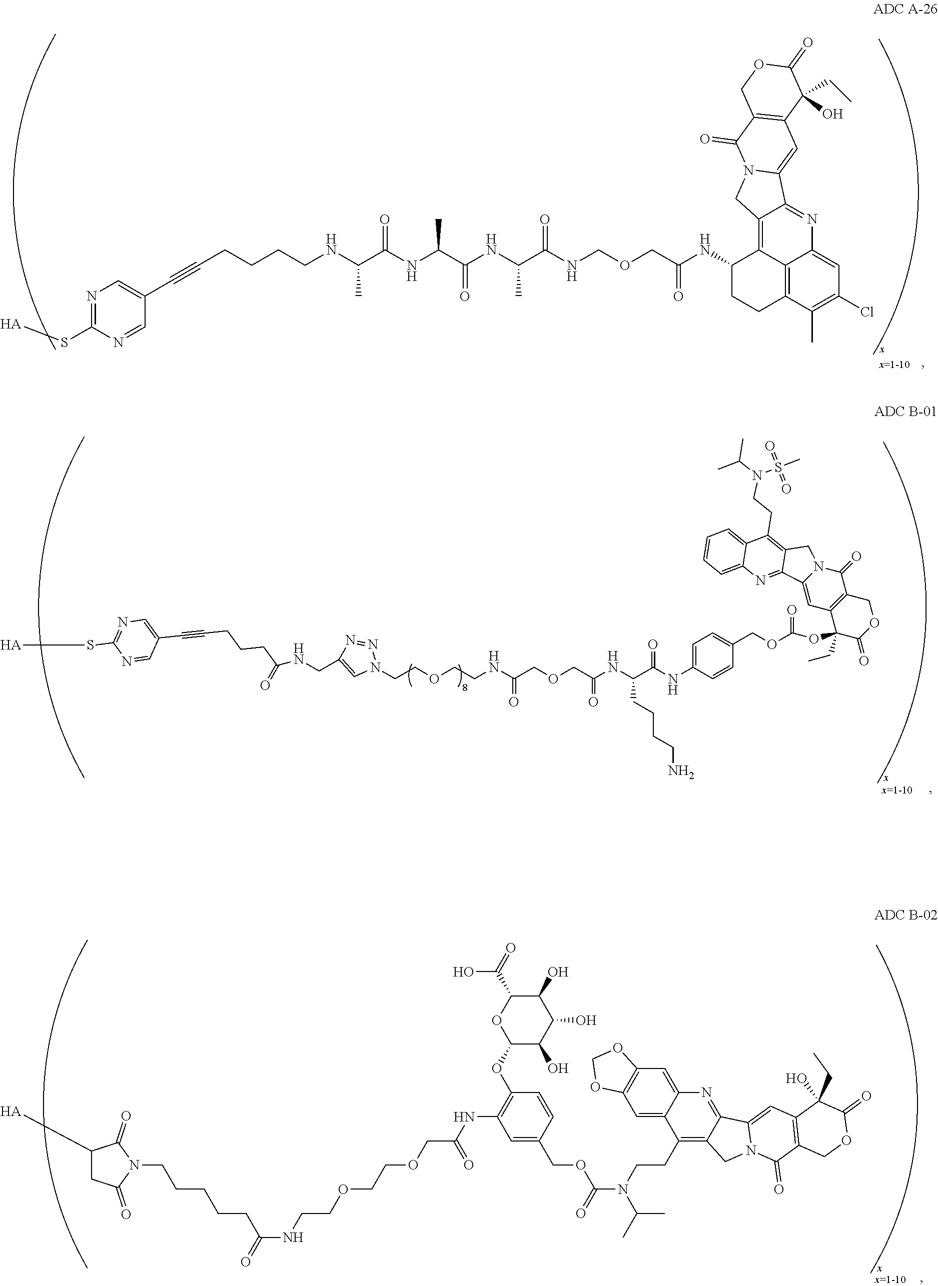

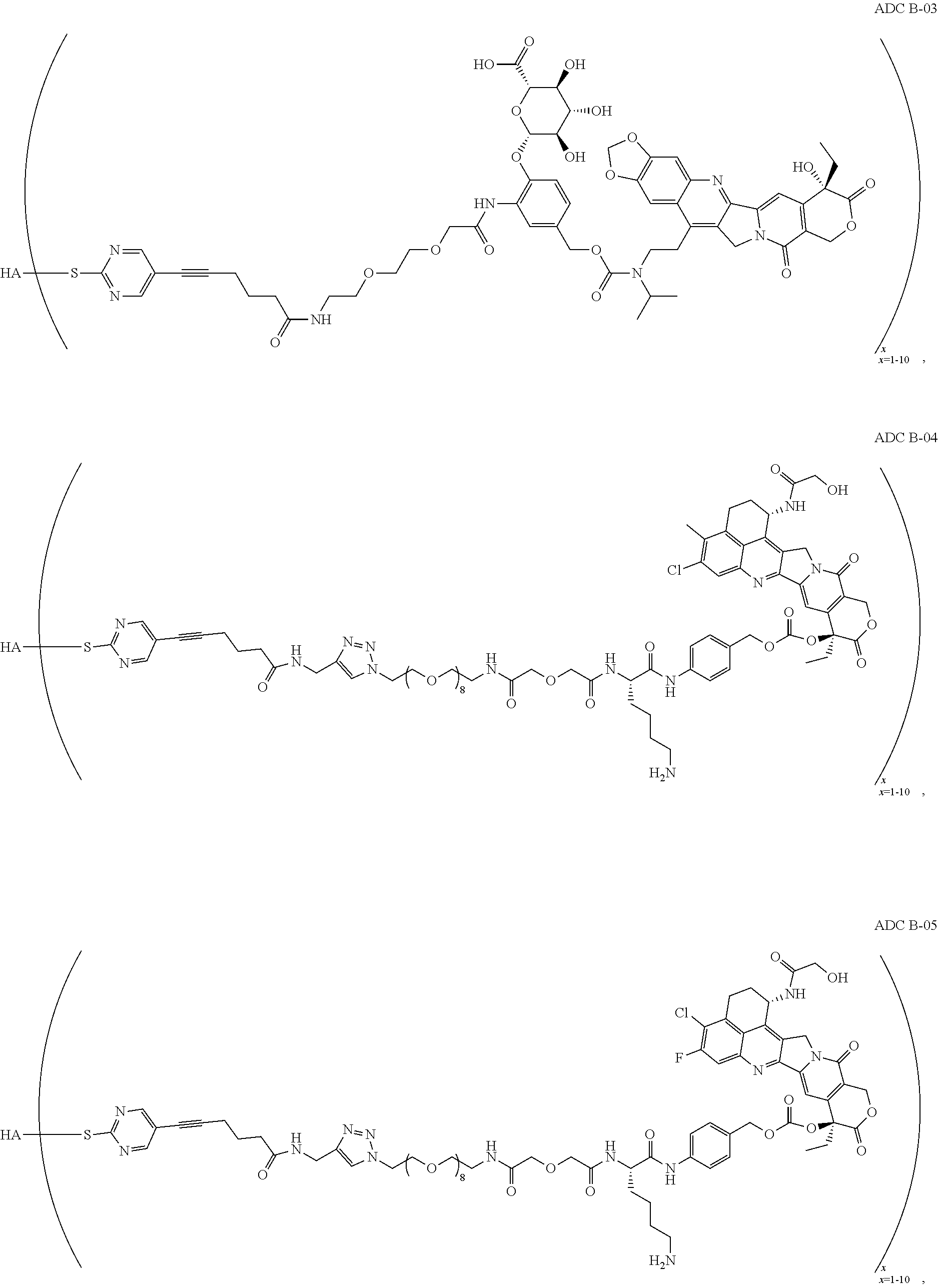

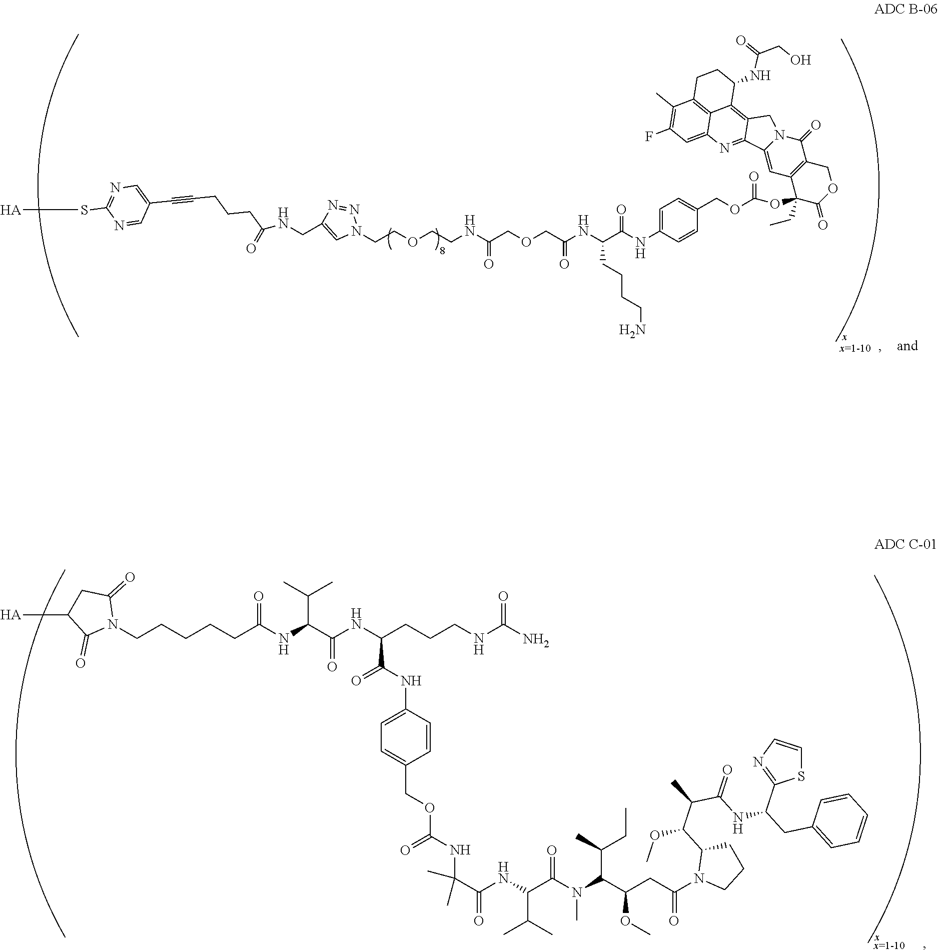

wherein HA in each antibody-drug conjugate represents antibodies or antigen-binding fragments of VH shown as SEQ ID NO: 1, 3, 5, 7 or 9 and VL shown as SEQ ID NO: 2, 4, 6, 8 or 10, for example the antibodies or antigen-binding fragments of VH shown as SEQ ID NO: 3 and CH shown as SEQ ID NO: 43 or 45, VL shown as SEQ ID NO: 4 and CL shown as SEQ ID NO: 44;

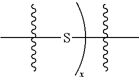 [SMILES: *S*]

represents linkage formed from a sulfhydryl in the antibody or antigen-binding fragment and the linker; wherein
HA is linked via the sulfhydryls in the antibody or antigen-binding fragment to form the antibody-drug conjugate.

In some embodiments, the antibody-drug conjugate is selected from:

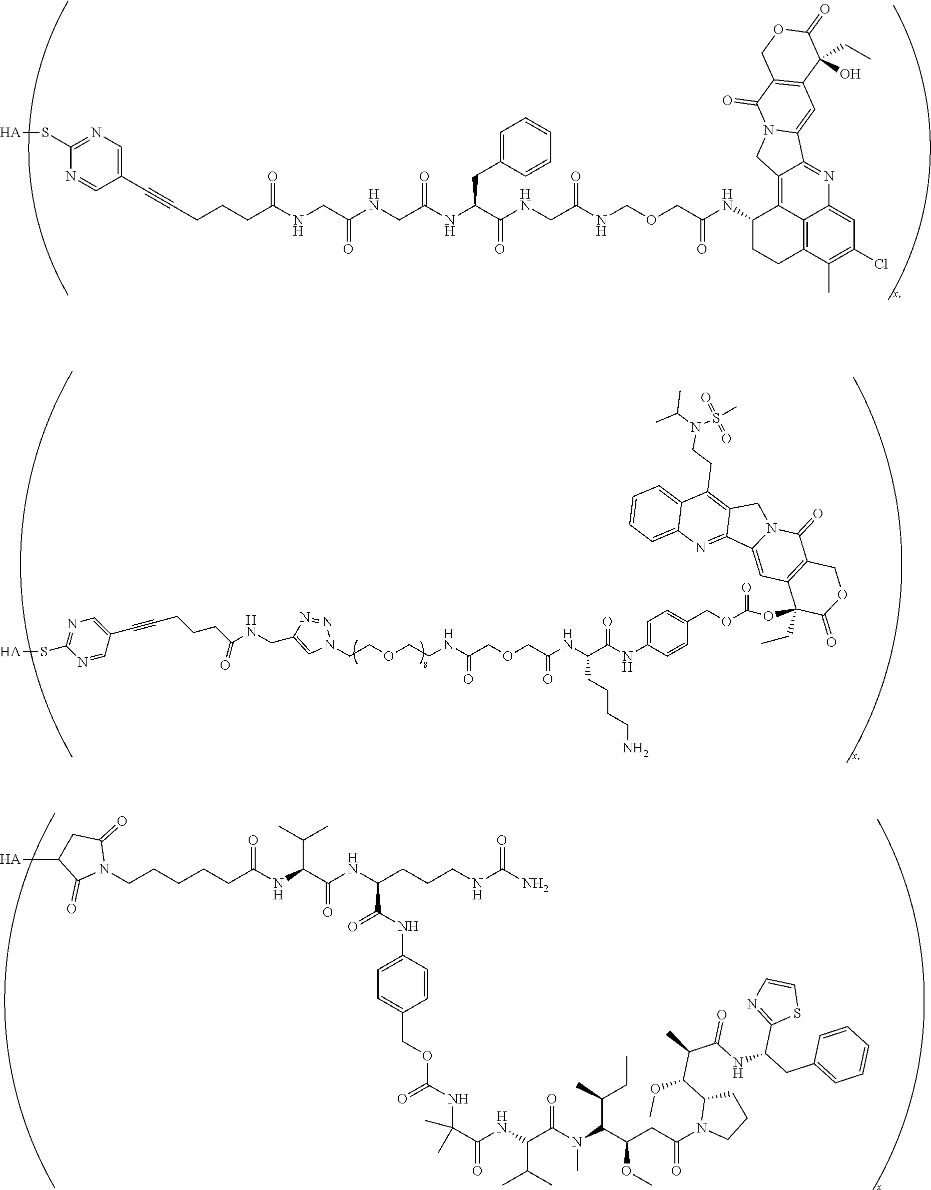

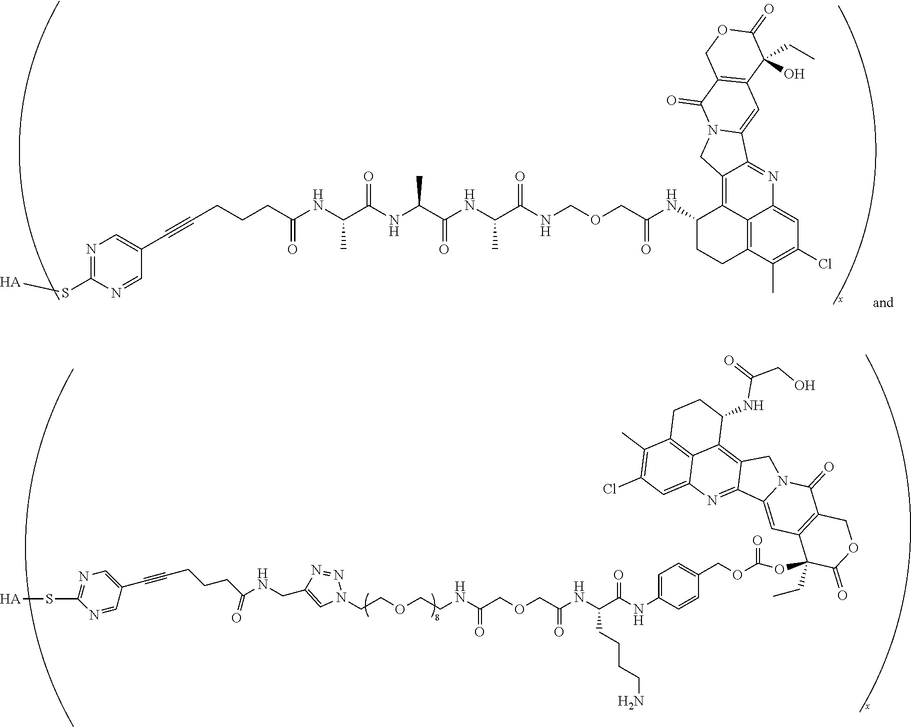

wherein the HA in each antibody-drug conjugate is selected from:
(1) an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44;
(2) an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44;
(3) an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44;
(4) an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44; and
(5) an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44,

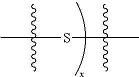 [SMILES: *S*]

represents linkage formed from a sulfhydryl in the antibody or antigen-binding fragment and the linker, wherein
HA is linked via the sulfhydryls in the antibody or antigen-binding fragment to form the antibody-drug conjugate,
and x is 3 to 9.

In some embodiments, the antibody-drug conjugate is selected from:

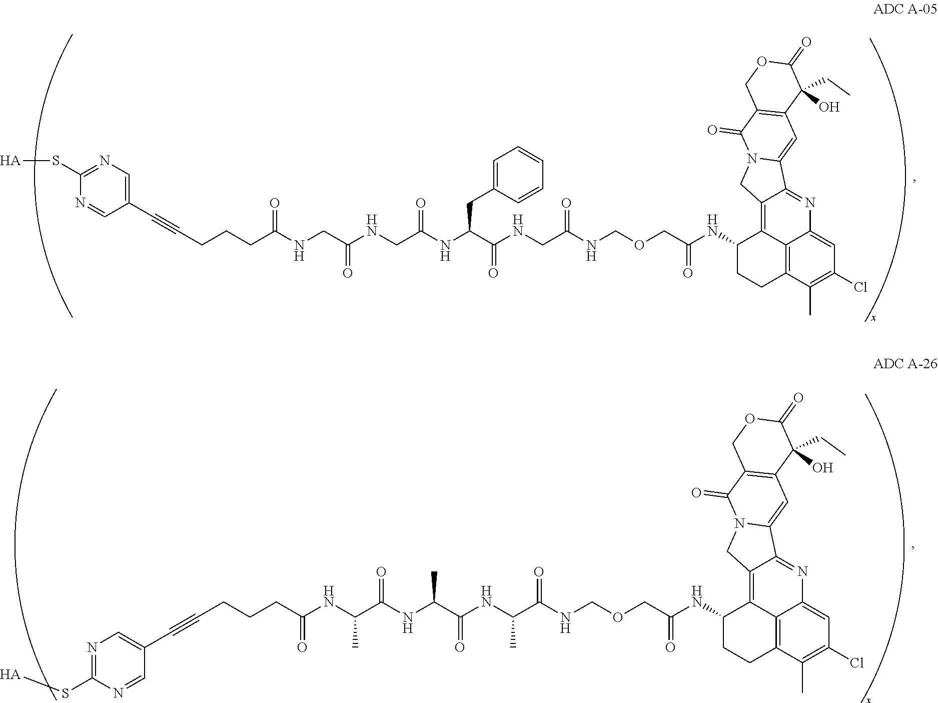

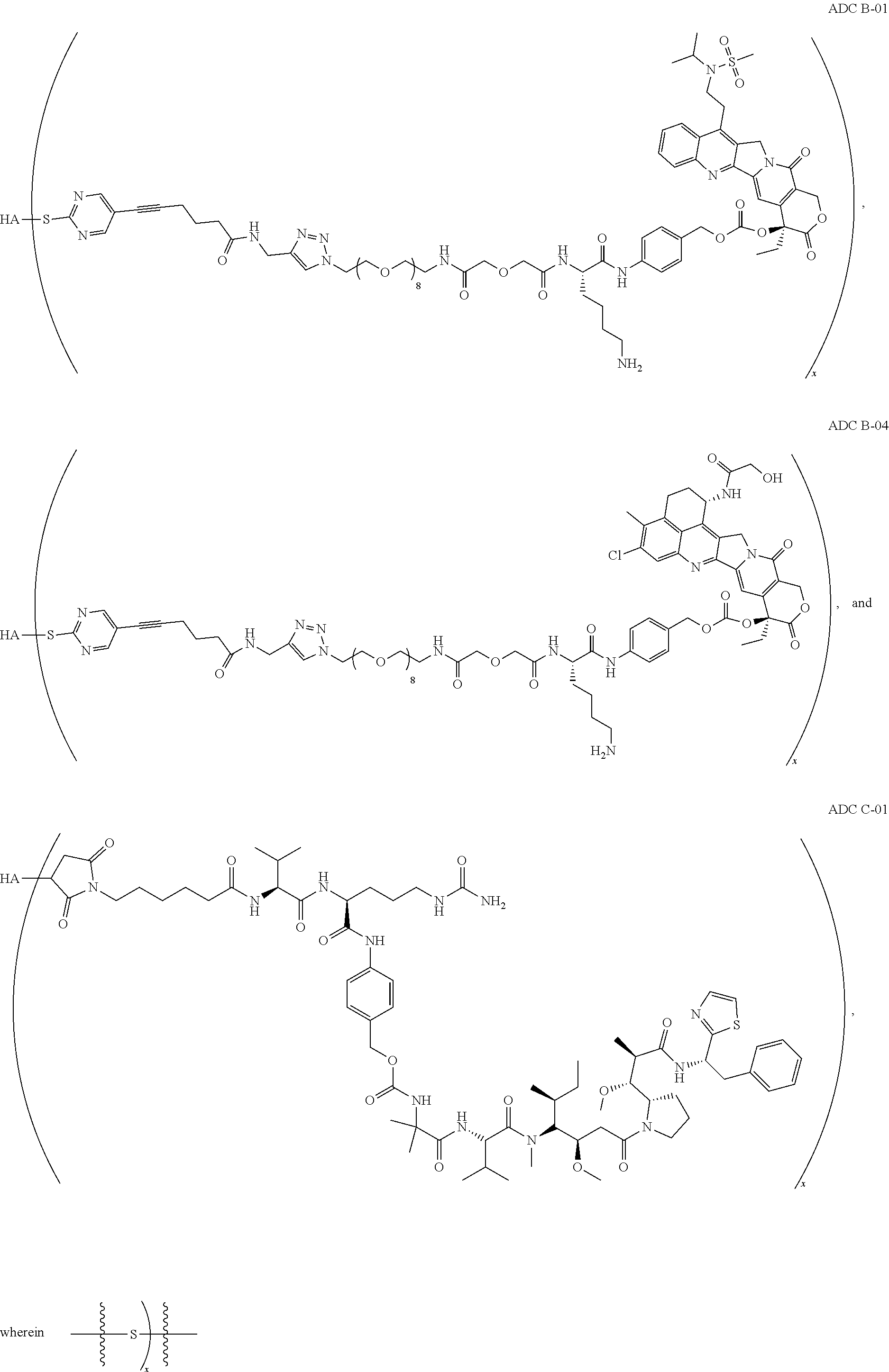

represents linkage formed from a sulfhydryl in the antibody or antigen-binding fragment and the linker, wherein
HA is linked via the sulfhydryls in the antibody or antigen-binding fragment to form the antibody-drug conjugate,
x is 2 to 8, 3 to 4, or 7 to 8, and HA in each antibody-drug conjugate is selected from an antibody or antigen binding fragment comprising:
(1) the following heavy chain variable regions (VH) and/or light chain variable regions (VL), wherein CDRs are defined according to a Chothia numbering system:
(1a) a heavy chain variable region (VH) comprising the following 3 CDRs: CDR-H1 having the amino acid sequence as set forth in SEQ ID NO: 11 or a variant thereof, CDR-H2 having the amino acid sequence as set forth in SEQ ID NO: 12 or a variant thereof, and CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(1b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (1a) and (1b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example replacement, deletion or addition of 1, 2 or 3 amino acids) compared with the sequence from which the variants are derived; preferably, the replacement is conservative replacement;
   or,
(2) the following heavy chain variable region (VH) and/or light chain variable region (VL), wherein the CDRs are defined according to the Kabat numbering system:
(2a) a heavy chain variable region (VH) comprising the following 3 CDRs: CDR-H1 having the amino acid sequence as set forth in SEQ ID NO: 17 or a variant thereof, CDR-H2 having the amino acid sequence as set forth in SEQ ID NO: 18 or 19 or a variant thereof, CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(2b) a heavy chain variable region (VH) comprising the following 3 CDRs: CDR-H1 having the amino acid sequence as set forth in SEQ ID NO: 33 or a variant thereof, CDR-H2 having the amino acid sequence as set forth in SEQ ID NO: 34 or 35 or a variant thereof, and CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (2a) and (2b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement;
   or,
(3) the following heavy chain variable regions (VH) and/or light chain variable regions (VL), wherein the CDRs are defined according to the IMGT numbering system:
(3a) a heavy chain variable region (VH) comprising the following 3 CDRs: CDR-H1 having the amino acid sequence as set forth in SEQ ID NO: 20 or a variant thereof, CDR-H2 having the amino acid sequence as set forth in SEQ ID NO: 21 or a variant thereof, and CDR-H3 having the amino acid sequence as set forth in SEQ ID NO: 22 or a variant thereof; and/or, a light chain variable region (VL) comprising the following 3 CDRs: CDR-L1 having the amino acid sequence as set forth in SEQ ID NO: 23 or a variant thereof, CDR-L2 having the amino acid sequence as set forth in SEQ ID NO: 24 or a variant thereof, and CDR-L3 having the amino acid sequence as set forth in SEQ ID NO: 16 or a variant thereof; or,
(3b) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, and CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 39 or a variant thereof, CDR-L2 having the amino acid sequence as set forth in SEQ ID NO: 40 or a variant thereof, and CDR-L3 having the amino acid sequence as set forth in SEQ ID NO: 32 or a variant thereof;
where the variants in any one of (3a) and (3b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement;
   or,
(4) the following heavy chain variable regions (VH) and/or light chain variable regions (VL), wherein the CDRs are defined according to the AbM numbering system:
(4a) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, and CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(4b) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, and CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (4a) and (4b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement.

In a further embodiment, the HA in each antibody-drug conjugate is selected from an antibody or antigen binding fragment comprising:
(a) a VH having the amino acid sequence as set forth in SEQ ID NO: 1 or a variant thereof, and/or, a VL having the amino acid sequence as set forth in SEQ ID NO: 2 or a variant thereof;
(b) a VH having the amino acid sequence as set forth in SEQ ID NO: 3 or a variant thereof, and/or, a VL having the amino acid sequence as set forth in SEQ ID NO: 4 or a variant thereof;
(c) a VH having the amino acid sequence as set forth in SEQ ID NO: 5 or a variant thereof, and/or, a VL having the amino acid sequence as set forth in SEQ ID NO: 6 or a variant thereof;
(d) a VH having the amino acid sequence as set forth in SEQ ID NO: 7 or a variant thereof, and/or, a VL having the amino acid sequence as set forth in SEQ ID NO: 8 or a variant thereof; and
(e) a VH having the amino acid sequence as set forth in SEQ ID NO: 9 or a variant thereof, and/or, a VL having the amino acid sequence as set forth in SEQ ID NO: 10 or a variant thereof;
wherein the variants described in any item of (a), (b), (c), (d) and (e) has the amino acid sequence identity of at least 70%, at least 80%, at least 85%, at least 90%, at least 91%, at least 92%, at least 93%, at least 94%, at least 95%, at least 96%, at least 97%, at least 98%, at least 99% or 100% as compared to the sequence from which the variants are derived, or are subjected to replacement, deletion or addition of one or several amino acids (for example replacement, deletion or addition of 1, 2, 3, 4 or 5 amino acids) as compared to the sequence from which the variants are derived; and preferably, the replacement is conservative replacement.

In a further embodiment, the HA in each antibody-drug conjugate is selected from an antibody or antigen binding fragment comprising:
(a) a VH having the amino acid sequence as set forth in SEQ ID NO: 1, and/or, a VL having the amino acid sequence as set forth in SEQ ID NO: 2;
(b) a VH having the amino acid sequence as set forth in SEQ ID NO: 3, and/or, a VL having the amino acid sequence as set forth in SEQ ID NO: 4;
(c) a VH having the amino acid sequence as set forth in SEQ ID NO: 5, and/or, a VL having the amino acid sequence as set forth in SEQ ID NO: 6;
(d) a VH having the amino acid sequence as set forth in SEQ ID NO: 7, and/or, a VL having the amino acid sequence as set forth in SEQ ID NO: 8; and
(e) a VH having the amino acid sequence as set forth in SEQ ID NO: 9, and/or, a VL having the amino acid sequence as set forth in SEQ ID NO: 10.

In a further embodiment, the HA in each antibody-drug conjugate is an antibody or antigen binding fragment comprising a heavy chain comprising a VH having the amino acid sequence selected from the group consisting of SEQ ID NOs: 1, 3, 5, 7, and 9.

In a further embodiment, the HA in each antibody-drug conjugate is an antibody or antigen binding fragment comprising a heavy chain comprising a VH having the amino acid sequence set forth in SEQ ID NO: 1.

In a further embodiment, the HA in each antibody-drug conjugate is an antibody or antigen binding fragment comprising a heavy chain comprising a VH having the amino acid sequence set forth in SEQ ID NO: 3.

In a further embodiment, the HA in each antibody-drug conjugate is an antibody or antigen binding fragment comprising a heavy chain comprising a VH having the amino acid sequence set forth in SEQ ID NO: 5.

In a further embodiment, the HA in each antibody-drug conjugate is an antibody or antigen binding fragment comprising a heavy chain comprising a VH having the amino acid sequence set forth in SEQ ID NO: 7.

In a further embodiment, the HA in each antibody-drug conjugate is an antibody or antigen binding fragment comprising a heavy chain comprising a VH having the amino acid sequence set forth in SEQ ID NO: 9.

In a further embodiment, the HA in each antibody-drug conjugate is an antibody or antigen binding fragment comprising a heavy chain comprising a VH having the amino acid sequence selected from the group consisting of SEQ ID NOs: 1, 3, 5, 7 and 9, wherein the N-terminal glutamine or glutamic acid of the antibody variable region has undergone cyclization to pyroglutamate.

In a further embodiment, the HA in each antibody-drug conjugate is an antibody or antigen binding fragment comprising a heavy chain comprising a VH having the amino acid sequence selected from the group consisting of SEQ ID NOs: 1, 3, 5, 7 and 9, wherein the N-terminal glutamine or glutamic acid of the antibody variable region has undergone cyclization to pyroglutamic acid.

In a further embodiment, the HA in each antibody-drug conjugate is an antibody or antigen binding fragment comprising a heavy chain comprising a VH having the amino acid sequence set forth in SEQ ID NO: 1, wherein the N-terminal glutamic acid of the antibody heavy chain variable region has undergone cyclization to pyroglutamate.

In a further embodiment, the HA in each antibody-drug conjugate is an antibody or antigen binding fragment comprising a heavy chain comprising a VH having the amino acid sequence set forth in SEQ ID NO: 1, wherein the N-terminal glutamic acid of the antibody heavy chain variable region has undergone cyclization to pyroglutamic acid.

In a further embodiment, the HA in each antibody-drug conjugate is an antibody or antigen binding fragment comprising a heavy chain comprising a VH having the amino acid sequence set forth in SEQ ID NO: 3, wherein the N-terminal glutamic acid of the antibody heavy chain variable region has undergone cyclization to pyroglutamate.

In a further embodiment, the HA in each antibody-drug conjugate is an antibody or antigen binding fragment comprising a heavy chain comprising a VH having the amino acid sequence set forth in SEQ ID NO: 3, wherein the N-terminal glutamic acid of the antibody heavy chain variable region has undergone cyclization to pyroglutamic acid.

In a further embodiment, the HA in each antibody-drug conjugate is an antibody or antigen binding fragment comprising a heavy chain comprising a VH having the amino acid sequence set forth in SEQ ID NO: 5, wherein the N-terminal glutamine of the antibody heavy chain variable region has undergone cyclization to pyroglutamate.

In a further embodiment, the HA in each antibody-drug conjugate is an antibody or antigen binding fragment comprising a heavy chain comprising a VH having the amino acid sequence set forth in SEQ ID NO: 5, wherein the N-terminal glutamine of the antibody heavy chain variable region has undergone cyclization to pyroglutamic acid.

In a further embodiment, the HA in each antibody-drug conjugate is an antibody or antigen binding fragment comprising a heavy chain comprising a VH having the amino acid sequence set forth in SEQ ID NO: 7, wherein the N-terminal glutamine of the antibody heavy chain variable region has undergone cyclization to pyroglutamate.

In a further embodiment, the HA in each antibody-drug conjugate is an antibody or antigen binding fragment comprising a heavy chain comprising a VH having the amino acid sequence set forth in SEQ ID NO: 7, wherein the N-terminal glutamine of the antibody heavy chain variable region has undergone cyclization to pyroglutamic acid.

In a further embodiment, the HA in each antibody-drug conjugate is an antibody or antigen binding fragment comprising a heavy chain comprising a VH having the amino acid sequence set forth in SEQ ID NO: 9, wherein the N-terminal glutamine of the antibody heavy chain variable region has undergone cyclization to pyroglutamate.

In a further embodiment, the HA in each antibody-drug conjugate is an antibody or antigen binding fragment comprising a heavy chain comprising a VH having the amino acid sequence set forth in SEQ ID NO: 9, wherein the N-terminal glutamine of the antibody heavy chain variable region has undergone cyclization to pyroglutamic acid.

In a further embodiment, the HA in each antibody-drug conjugate is an antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises:
(a) a VH having the amino acid sequence set forth in SEQ ID NO: 1 or a variant thereof and/or a VL having the amino acid sequence set forth in SEQ ID NO: 2 or a variant thereof;
(b) a VH having the amino acid sequence set forth in SEQ ID NO: 3 or a variant thereof and/or a VL having the amino acid sequence set forth in SEQ ID NO: 4 or a variant thereof;
(c) a VH having the amino acid sequence set forth in SEQ ID NO: 5 or a variant thereof and/or a VL having the amino acid sequence set forth in SEQ ID NO: 6 or a variant thereof;
(d) a VH having the amino acid sequence set forth in SEQ ID NO: 7 or a variant thereof and/or a VL having the amino acid sequence set forth in SEQ ID NO: 8 or a variant thereof; or
(e) a VH having the amino acid sequence set forth in SEQ ID NO: 9 or a variant thereof and/or a VL having the amino acid sequence set forth in SEQ ID NO: 10 or a variant thereof;
wherein the variants have at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids (such as replacement, deletion or addition of 1, 2, 3, 4 or 5 amino acids) as compared to the sequence from which the variants are derived.

In a further embodiment, the HA in each antibody-drug conjugate is an antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises:
(a) a VH having the amino acid sequence set forth in SEQ ID NO: 1 and/or a VL having the amino acid sequence set forth in SEQ ID NO: 2;
(b) a VH having the amino acid sequence set forth in SEQ ID NO: 3 and/or a VL having the amino acid sequence set forth in SEQ ID NO: 4;
(c) a VH having the amino acid sequence set forth in SEQ ID NO: 5 and/or a VL having the amino acid sequence set forth in SEQ ID NO: 6;
(d) a VH having the amino acid sequence set forth in SEQ ID NO: 7 and/or a VL having the amino acid sequence set forth in SEQ ID NO: 8; or
(e) a VH having the amino acid sequence set forth in SEQ ID NO: 9 and/or a VL having the amino acid sequence set forth in SEQ ID NO: 10.

In a further embodiment, the HA in each antibody-drug conjugate is an antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises: (a) a VH having the amino acid sequence set forth in SEQ ID NO: 1 or a variant thereof and/or a VL having the amino acid sequence set forth in SEQ ID NO: 2 or a variant thereof.

In a further embodiment, the HA in each antibody-drug conjugate is an antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises: (b) a VH having the amino acid sequence set forth in SEQ ID NO: 3 or a variant thereof and/or a VL having the amino acid sequence set forth in SEQ ID NO: 4 or a variant thereof.

In a further embodiment, the HA in each antibody-drug conjugate is an antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises: (c) a VH having the amino acid sequence set forth in SEQ ID NO: 5 or a variant thereof and/or a VL having the amino acid sequence set forth in SEQ ID NO: 6 or a variant thereof.

In a further embodiment, the HA in each antibody-drug conjugate is an antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises: (d) a VH having the amino acid sequence set forth in SEQ ID NO: 7 or a variant thereof and/or a VL having the amino acid sequence set forth in SEQ ID NO: 8 or a variant thereof.

In a further embodiment, the HA in each antibody-drug conjugate is an antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises: (e) a VH having the amino acid sequence set forth in SEQ ID NO: 9 or a variant thereof and/or a VL having the amino acid sequence set forth in SEQ ID NO: 10 or a variant thereof.

In a further embodiment, the antibody-drug conjugate is selected from:

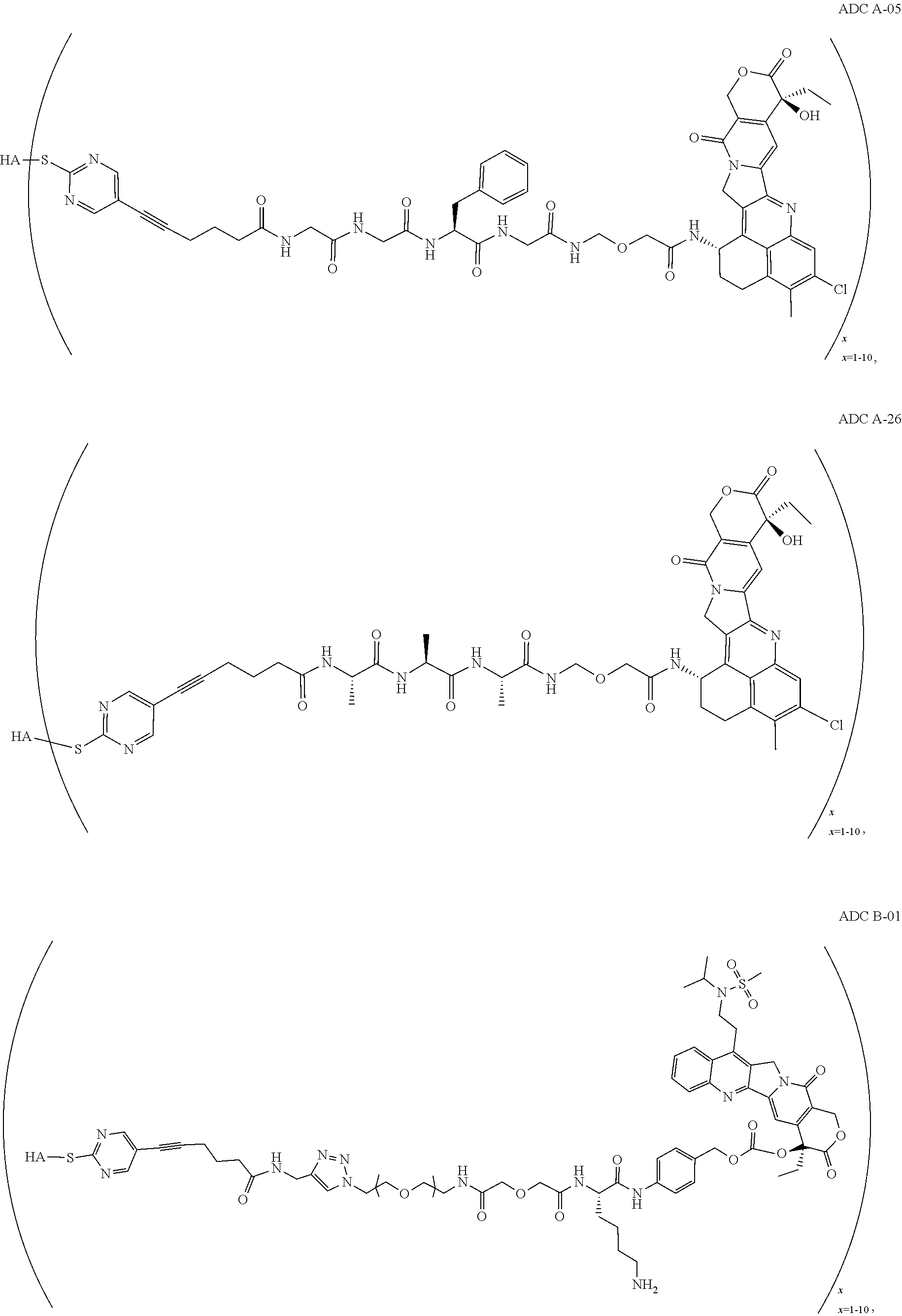

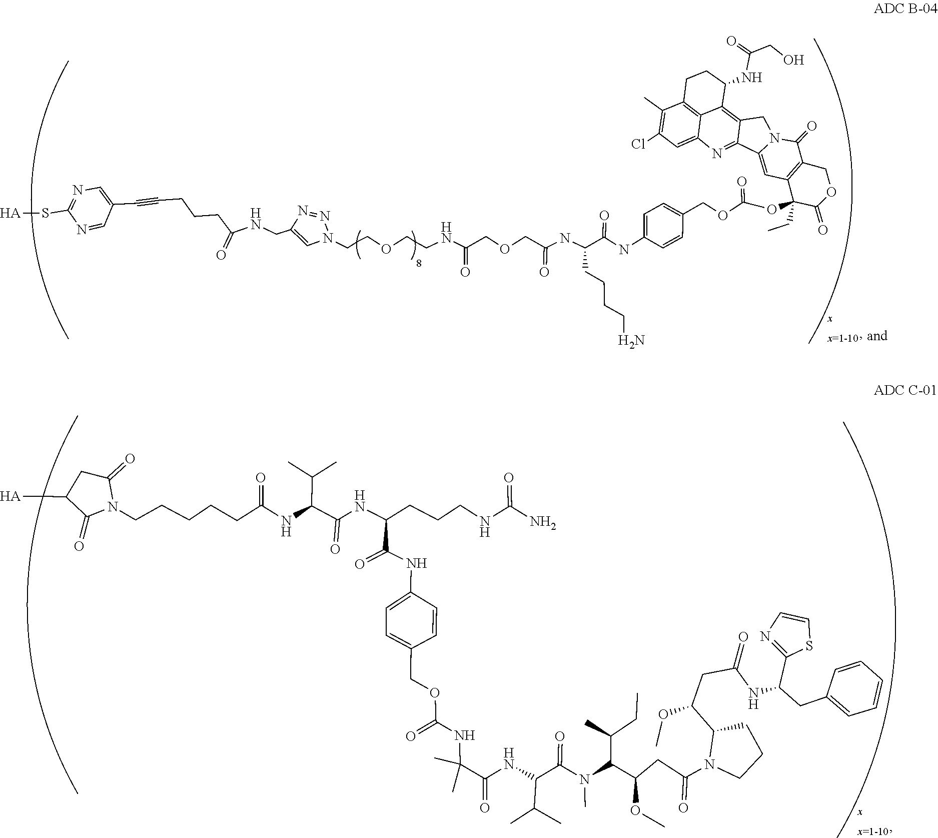

wherein
the HA in each antibody-drug conjugate is selected from:
(1) an antibody or antigen-binding fragment thereof comprising a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44;
(2) an antibody or antigen-binding fragment thereof comprising a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44;
(3) an antibody or antigen-binding fragment thereof comprising a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44;
(4) an antibody or antigen-binding fragment thereof comprising a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44; or
(5) an antibody or antigen-binding fragment thereof comprising a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44,

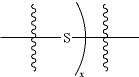 [SMILES: *S*]

represents linkage formed from a sulfhydryl in the antibody or antigen-binding fragment and the linker, wherein
HA is linked via the sulfhydryls in the antibody or antigen-binding fragment to form the antibody-drug conjugate.

In a further embodiment, the antibody-drug conjugate is:

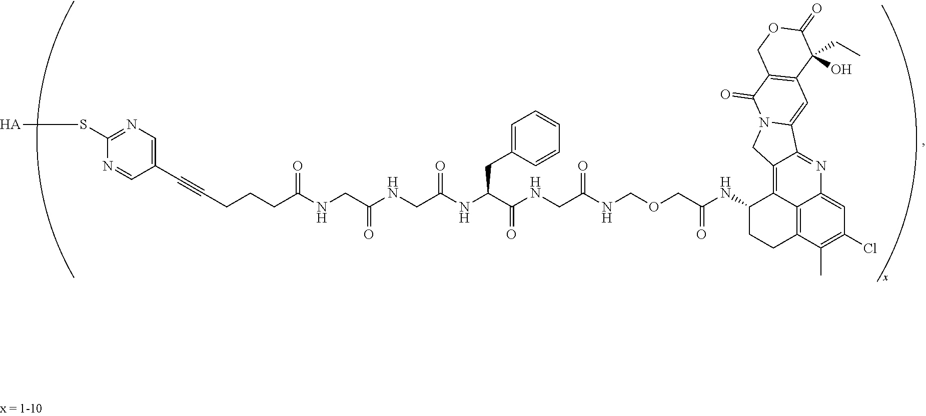

wherein the HA in each antibody-drug conjugate is selected from:
(1) an antibody or antigen-binding fragment thereof comprising a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44;
(2) an antibody or antigen-binding fragment thereof comprising a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44;
(3) an antibody or antigen-binding fragment thereof comprising a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44;
(4) an antibody or antigen-binding fragment thereof comprising a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44; and
(5) an antibody or antigen-binding fragment thereof comprising a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44,

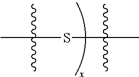 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

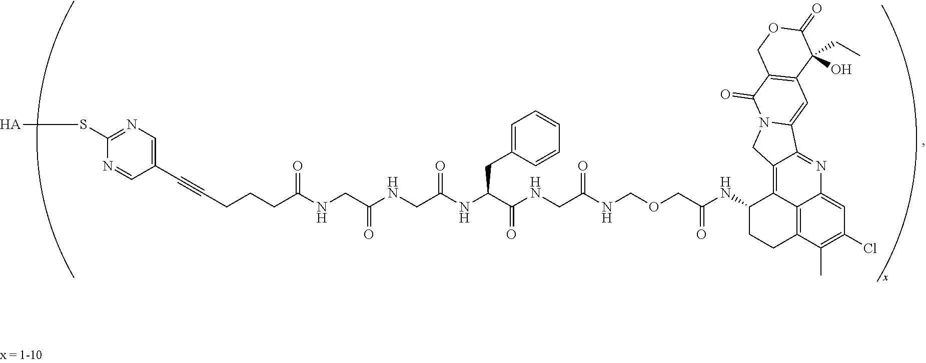

wherein the HA in the antibody-drug conjugate is an antibody or antigen binding fragment comprising: (1) a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44,

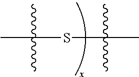 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

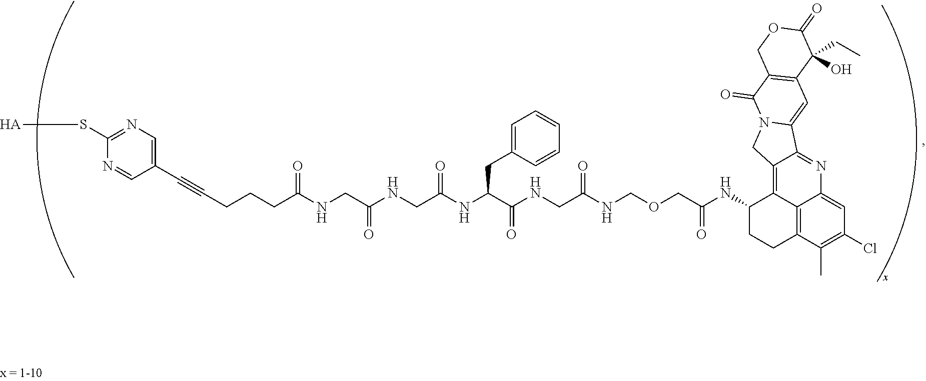

wherein the HA in the antibody-drug conjugate is an antibody or antigen binding fragment comprising: (2) a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44,

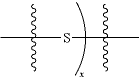 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

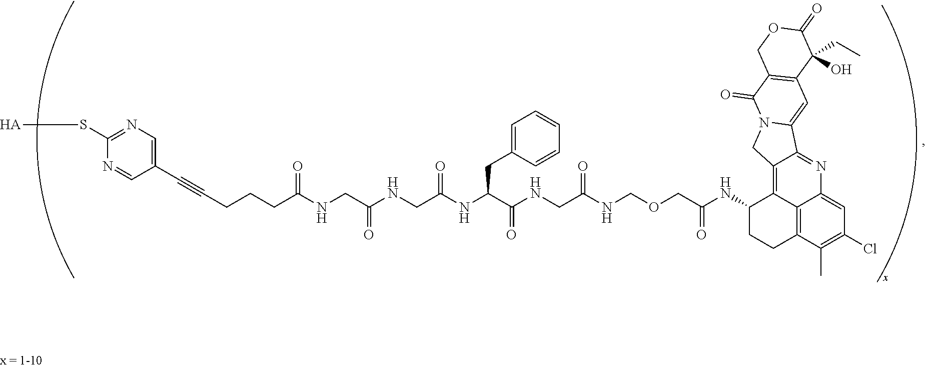

wherein the HA in the antibody-drug conjugate is an antibody or antigen binding fragment comprising: (3) a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44,

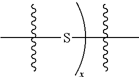 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

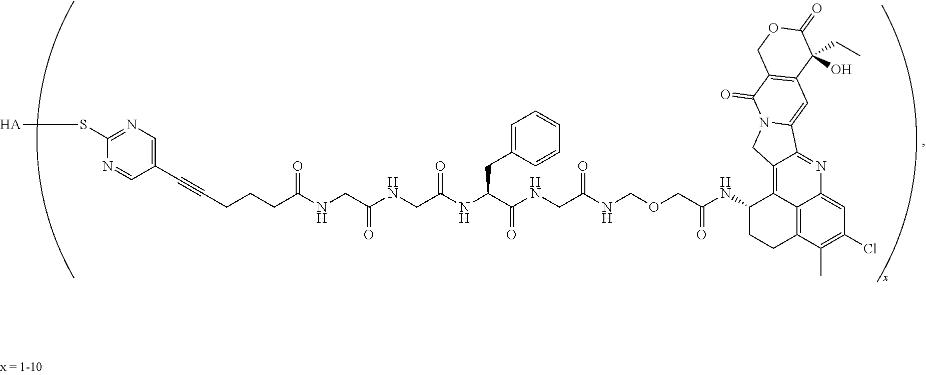

wherein the HA in the antibody-drug conjugate is an antibody or antigen binding fragment comprising: (4) a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44,

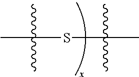 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

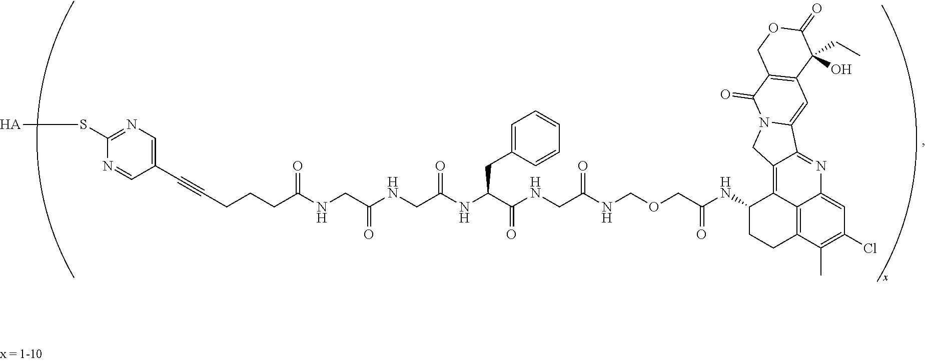

wherein the HA in the antibody-drug conjugate is an antibody or antigen binding fragment comprising: (5) a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44,

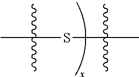 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

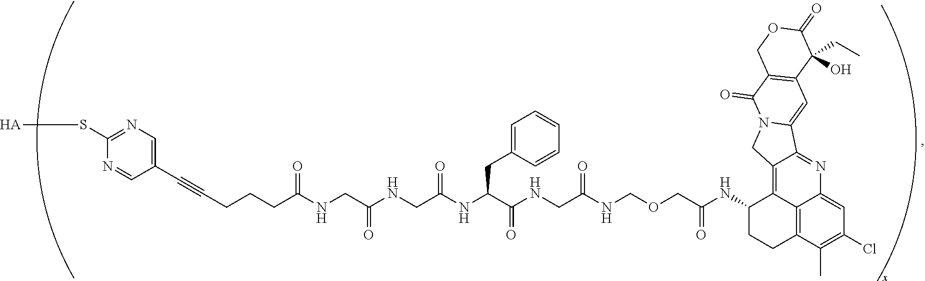

wherein the HA in the antibody-drug conjugate is an antibody or antigen-binding fragment thereof comprising: a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a heavy chain constant region (CH) having the amino acid sequence as set forth in SEQ ID NO: 43 or 45, and a light chain constant region (CL) having the amino acid sequence as set forth in SEQ ID NO: 44;
x is 3 to 4 or 7 to 9

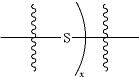 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

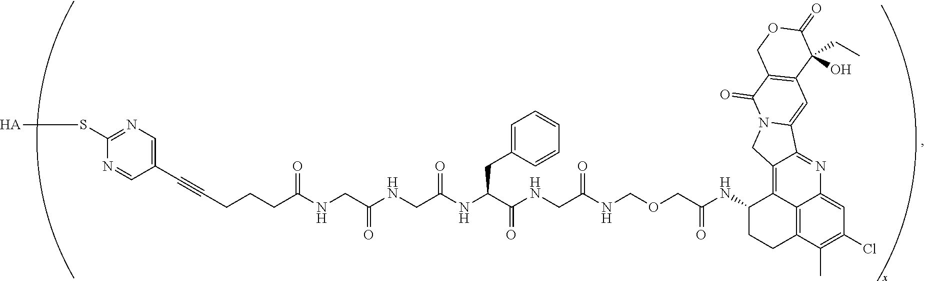

wherein the HA in the antibody-drug conjugate is an antibody or antigen-binding fragment thereof comprising: a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 44; and x is 7 to 9,

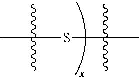 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

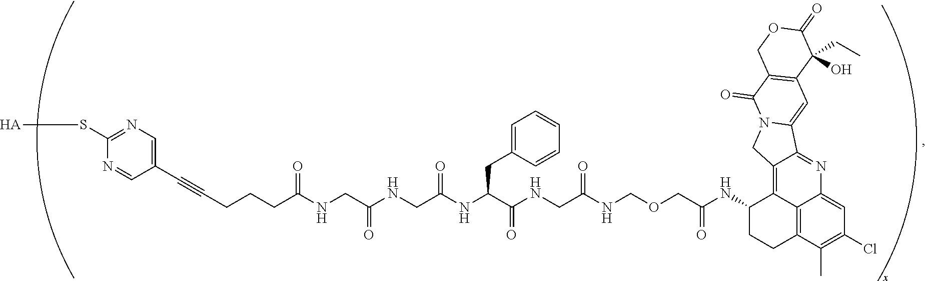

wherein the HA in the antibody-drug conjugate is an antibody or antigen-binding fragment thereof comprising: a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 44; and x is 8 to 9,

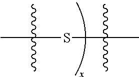 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

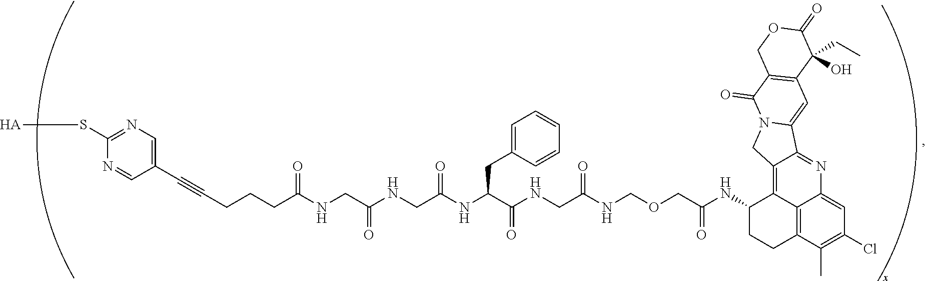

wherein the HA in the antibody-drug conjugate is an antibody or antigen-binding fragment thereof comprising: a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 44; and x is 8,

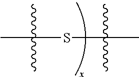 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

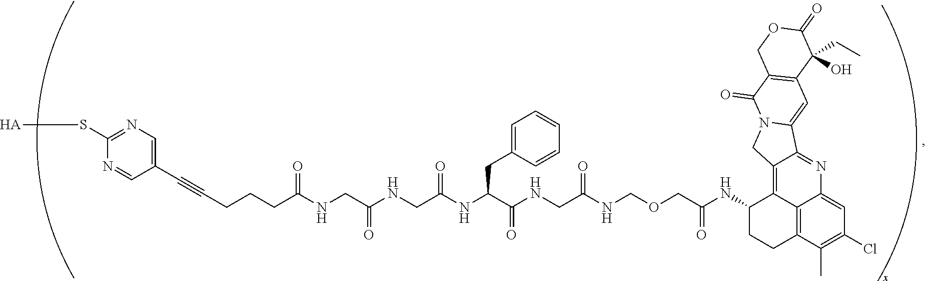

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 3 to 4 or 7 to 9,

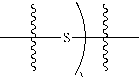 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

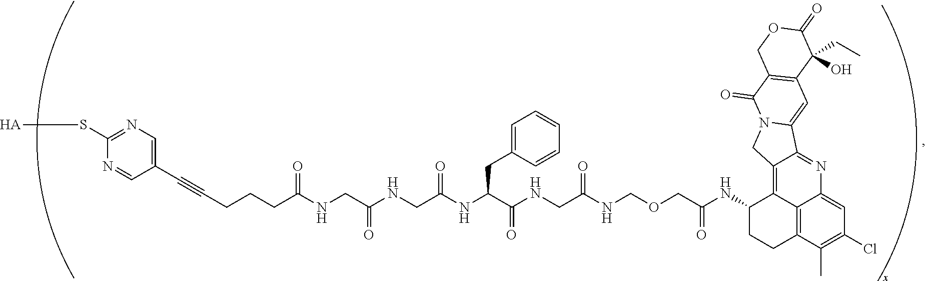

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 8 to 9,

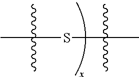 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

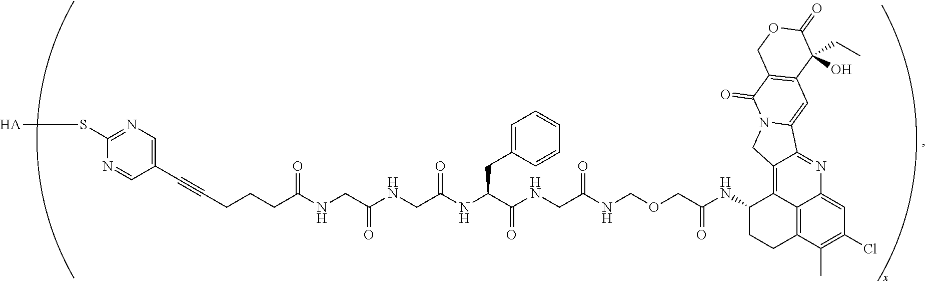

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 7 to 8,

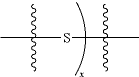 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

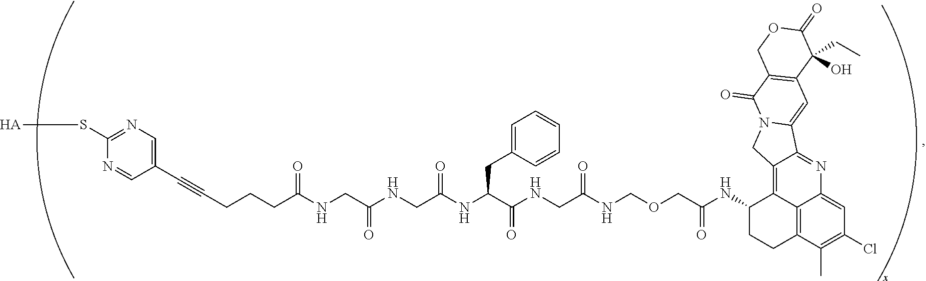

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47;
x is 7 to 8,

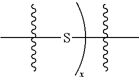 [SMILES: *S*]

represents linkage formed from a sulfhydryl in the antibody or antigen-binding fragment and the linker, and
HA is linked via the sulfhydryls of the cysteines in the antibody or antigen-binding fragment to form the antibody-drug conjugate.

In a further embodiment, the antibody-drug conjugate is:

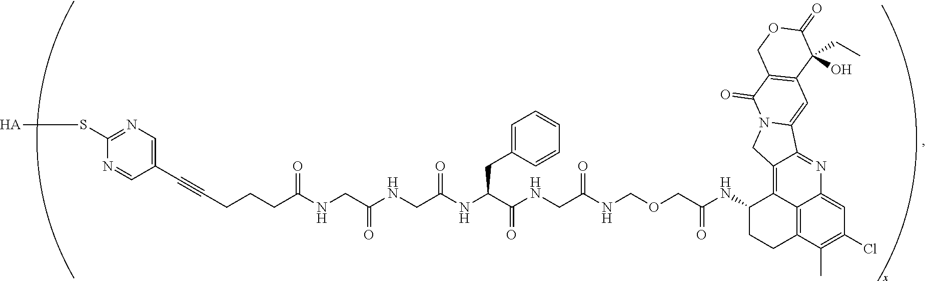

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 8,

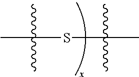 [SMILES: *S*]

represents linkage formed from a sulfhydryl in the antibody or antigen-binding fragment and the linker, and
HA is linked via the sulfhydryls of the cysteines in the antibody or antigen-binding fragment to form the antibody-drug conjugate.

In a further embodiment, the antibody-drug conjugate is:

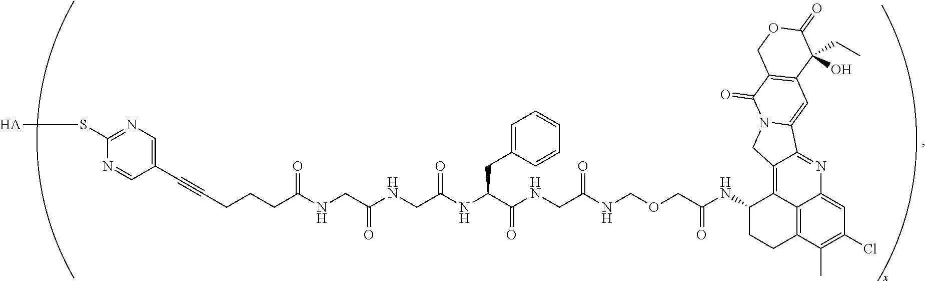

wherein HA is an antibody comprising a heavy chain (HC) consisting of the amino acid sequence as set forth in SEQ ID NO: 52, 53 or 54 and a light chain (LC) consisting of the amino acid sequence as set forth in SEQ ID NO: 47;

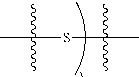 [SMILES: *S*]

represents linkage of formed from a sulfhydryl in the antibody or antigen-binding fragment and the linker,
x is 7 to 8, and
HA is linked via the sulfhydryls of the cysteines in the antibody or antigen-binding fragment to form the antibody-drug conjugate.

In a further embodiment, the antibody-drug conjugate is:

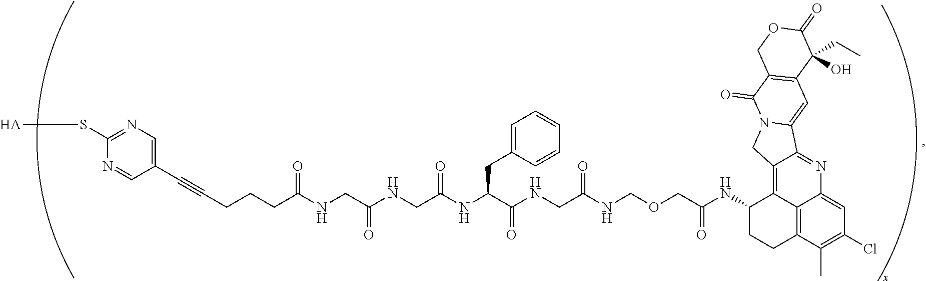

wherein HA is an antibody consisting of two heavy chains and two light chains, each heavy chain (HC) consists of the amino acid sequence as set forth in SEQ ID NO: 52 and a each light chain (LC) consists of the amino acid sequence as set forth in SEQ ID NO: 47;

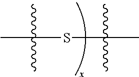 [SMILES: *S*]

represents linkage formed from a sulfhydryl in the antibody or antigen-binding fragment and the linker,
x is 7 to 8, and
HA is linked via the sulfhydryls of the cysteines in the antibody or antigen-binding fragment to form the antibody-drug conjugate.

In a further embodiment, the antibody-drug conjugate is:

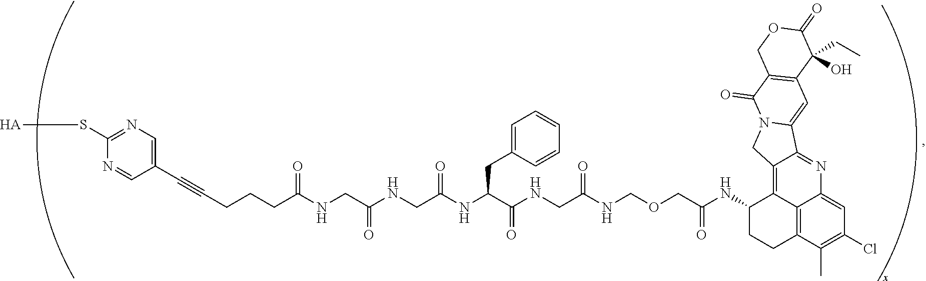

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47;

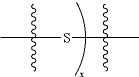 [SMILES: *S*]

represents linkage formed from a sulfhydryl in the antibody or antigen-binding fragment and the linker; wherein
HA is linked via the sulfhydryls of the cysteines in the antibody or antigen-binding fragment to form the antibody-drug conjugate;
and x is 8.

In a further embodiment, the antibody-drug conjugate is:

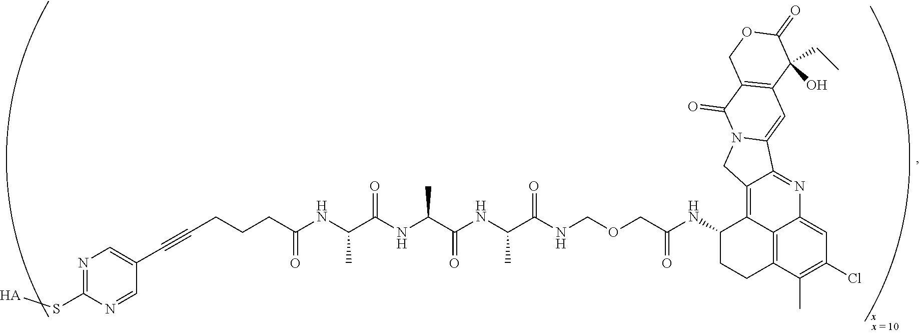

wherein the HA in each antibody-drug conjugate is selected from:
(1) an antibody or antigen-binding fragment thereof comprising a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44;
(2) an antibody or antigen-binding fragment thereof comprising a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44;
(3) an antibody or antigen-binding fragment thereof comprising a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44;
(4) an antibody or antigen-binding fragment thereof comprising a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44; and
(5) an antibody or antigen-binding fragment thereof comprising a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44,

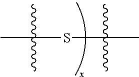 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

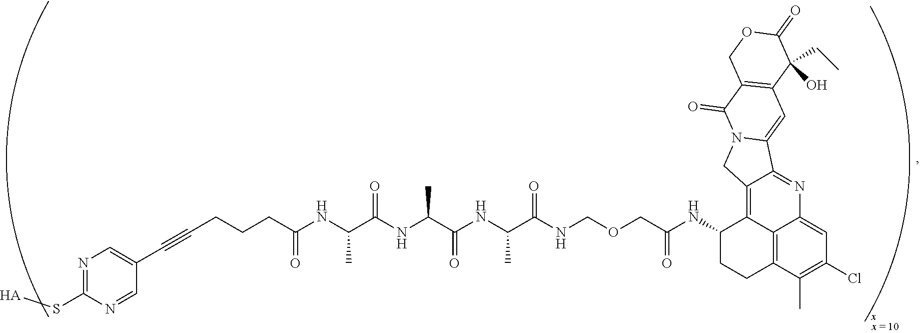

wherein the HA in the antibody-drug conjugate is an antibody or antigen binding fragment comprising: (1) a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44,

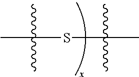 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

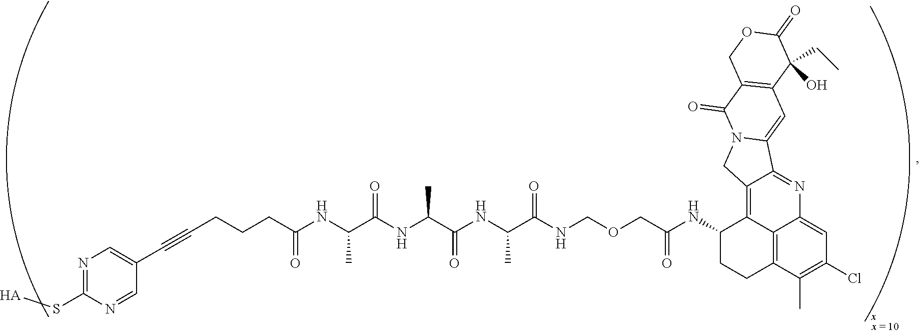

wherein the HA in the antibody-drug conjugate is an antibody or antigen binding fragment comprising: (2) a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44,

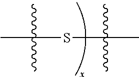 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

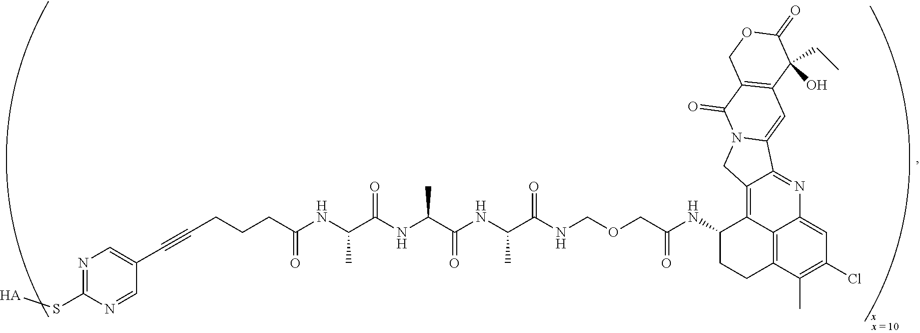

wherein the HA in the antibody-drug conjugate is an antibody or antigen binding fragment comprising: (3) a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44,

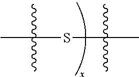 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

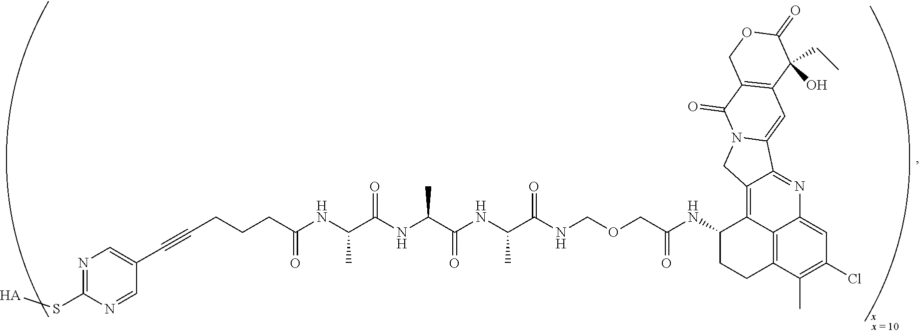

wherein the HA in the antibody-drug conjugate is an antibody or antigen binding fragment comprising: (4) a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44,

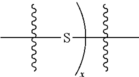 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

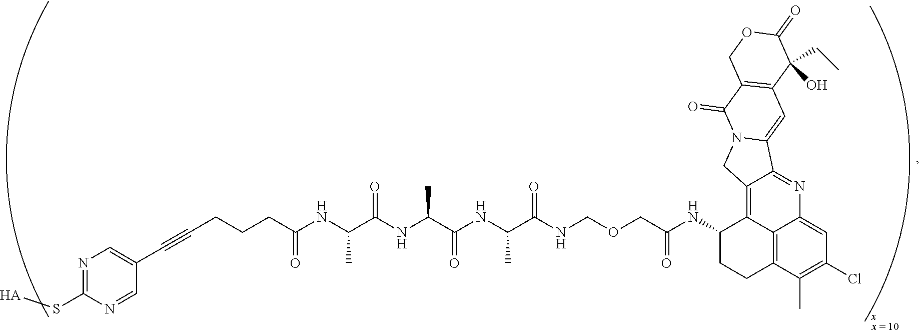

wherein the HA in the antibody-drug conjugate is an antibody or antigen binding fragment comprising: (5) a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44,

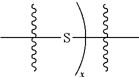 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

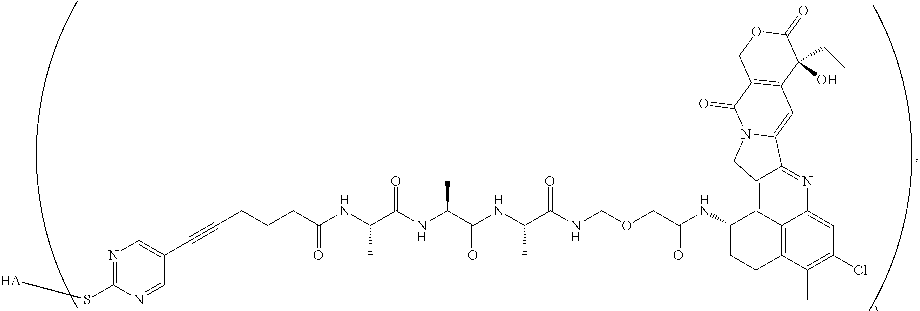

wherein the HA in the antibody-drug conjugate is an antibody or antigen-binding fragment thereof comprising: a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a heavy chain constant region (CH) having the amino acid sequence as set forth in SEQ ID NO: 43 or 45, and a light chain constant region (CL) having the amino acid sequence as set forth in SEQ ID NO: 44;
x is 7 to 9,

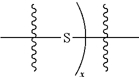 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

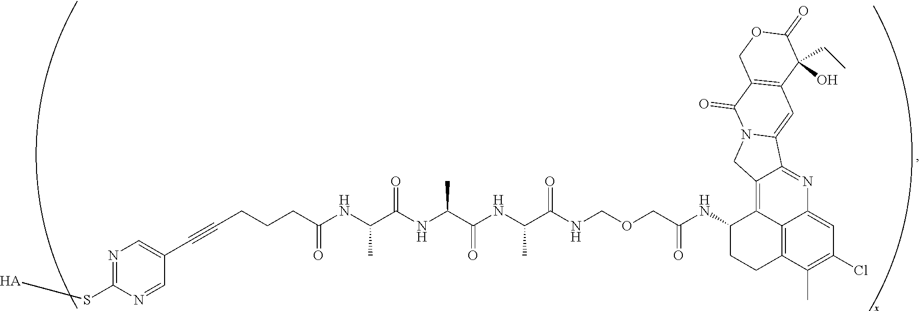

wherein the HA in the antibody-drug conjugate is an antibody or antigen-binding fragment thereof comprising: a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 44;
x is 7 to 8,

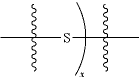 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

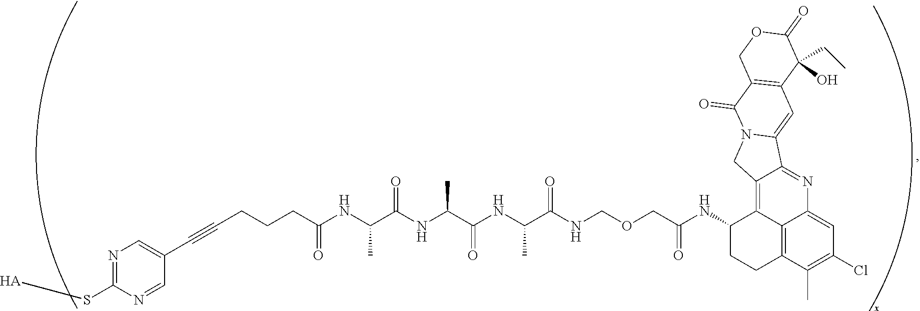

wherein the HA in the antibody-drug conjugate is an antibody or antigen-binding fragment thereof comprising: a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 44;
x is 8,

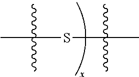 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

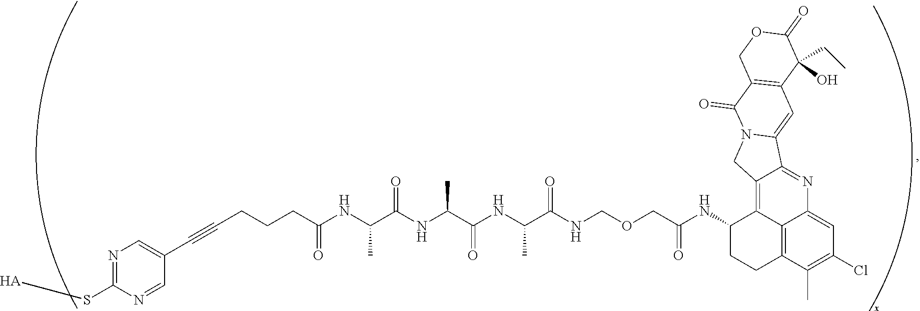

wherein HA in the antibody-drug conjugate is an antibody or antigen-binding fragment thereof comprising: a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 44;
x is 7 to 9,

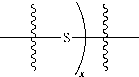 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

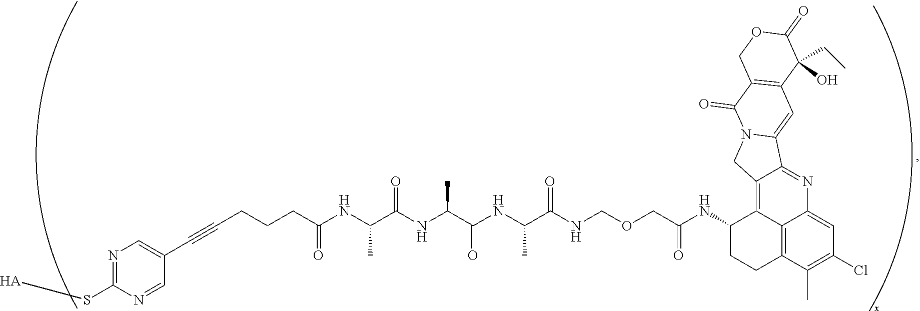

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47;
x is 7 to 8,

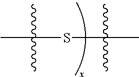 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

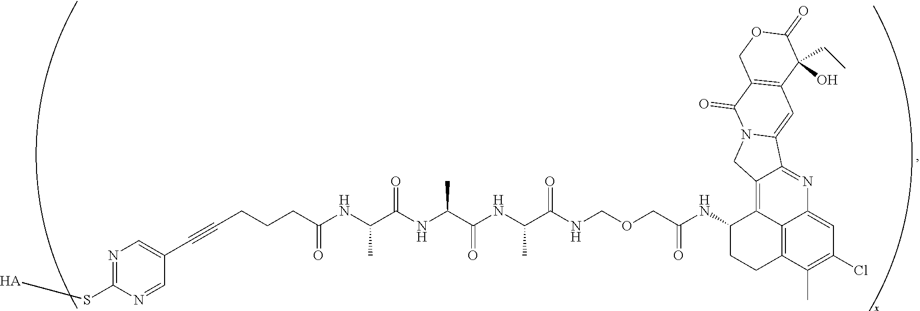

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47;
x is 8,

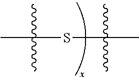 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

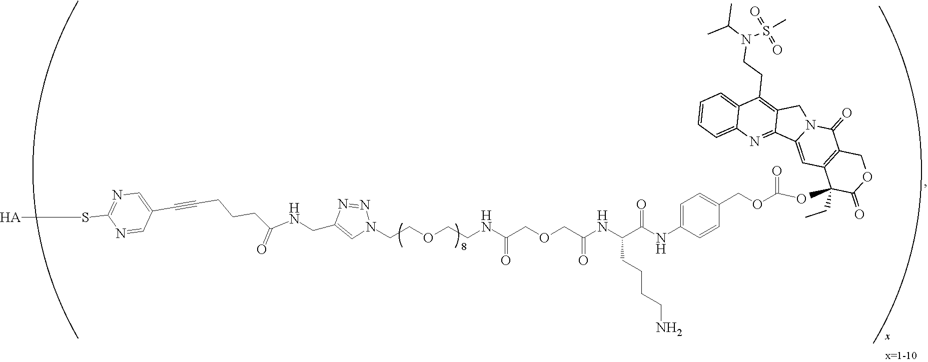

wherein the HA in the antibody-drug conjugate is selected from:
(1) an antibody or antigen-binding fragment thereof comprising a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44;
(2) an antibody or antigen-binding fragment thereof comprising a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44;
(3) an antibody or antigen-binding fragment thereof comprising a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44;
(4) an antibody or antigen-binding fragment thereof comprising a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44; and
(5) an antibody or antigen-binding fragment thereof comprising a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44.

In a further embodiment, the antibody-drug conjugate is:

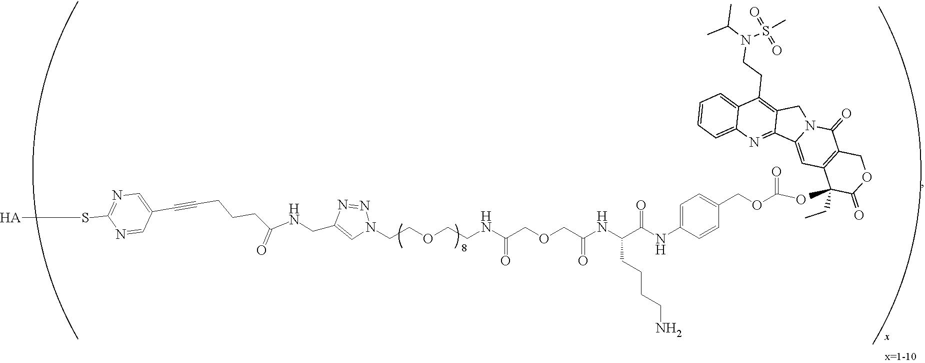

wherein the HA in the antibody-drug conjugate is an antibody or antigen binding fragment comprising: (1) a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44,

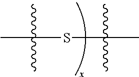 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

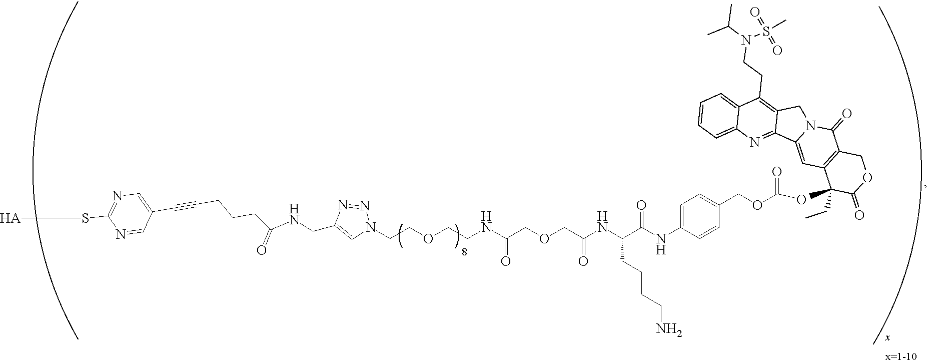

wherein the HA in the antibody-drug conjugate is an antibody or antigen binding fragment comprising: (2) a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44,

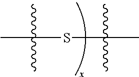 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

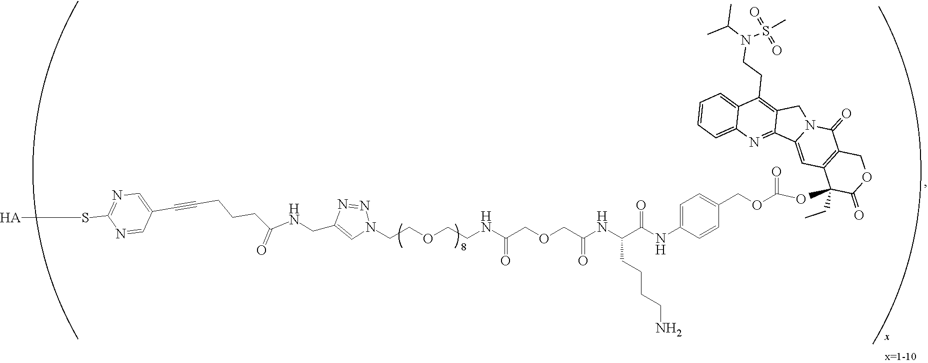

wherein the HA in the antibody-drug conjugate is an antibody or antigen binding fragment comprising: (3) a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44,

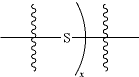 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

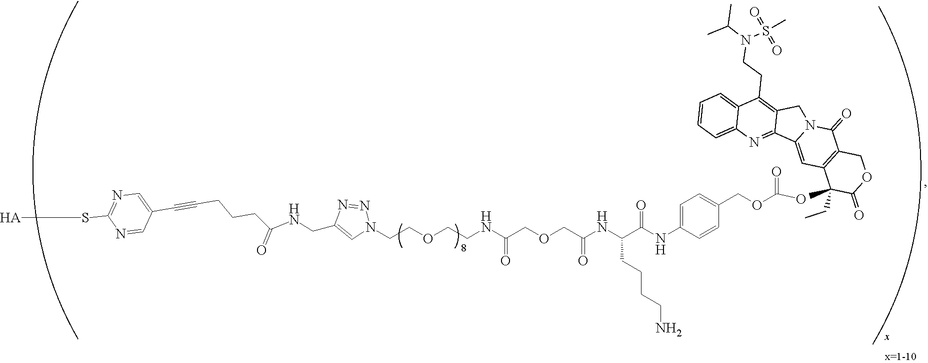

wherein the HA in the antibody-drug conjugate is an antibody or antigen binding fragment comprising: (4) a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44,

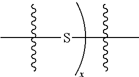 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

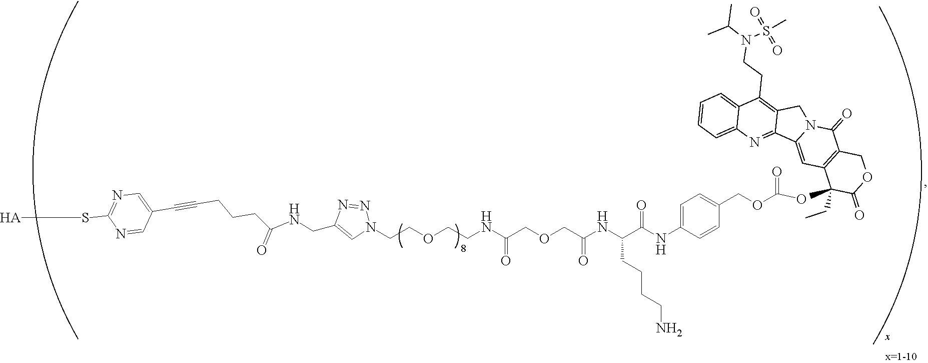

wherein the HA in the antibody-drug conjugate is an antibody or antigen binding fragment comprising: (5) a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44,

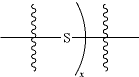 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

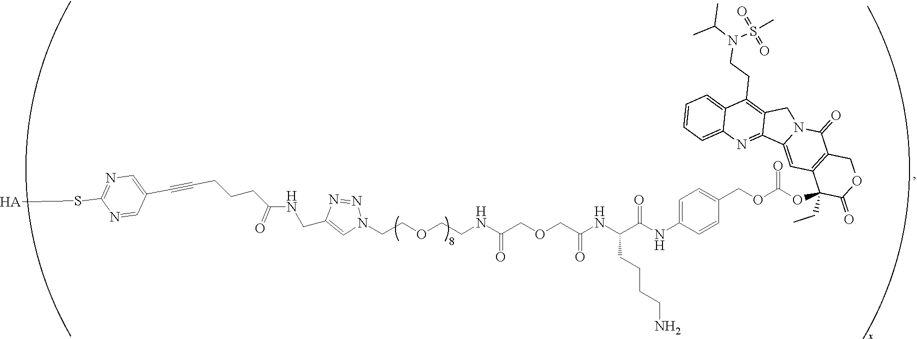

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 7 to 8,

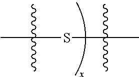 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

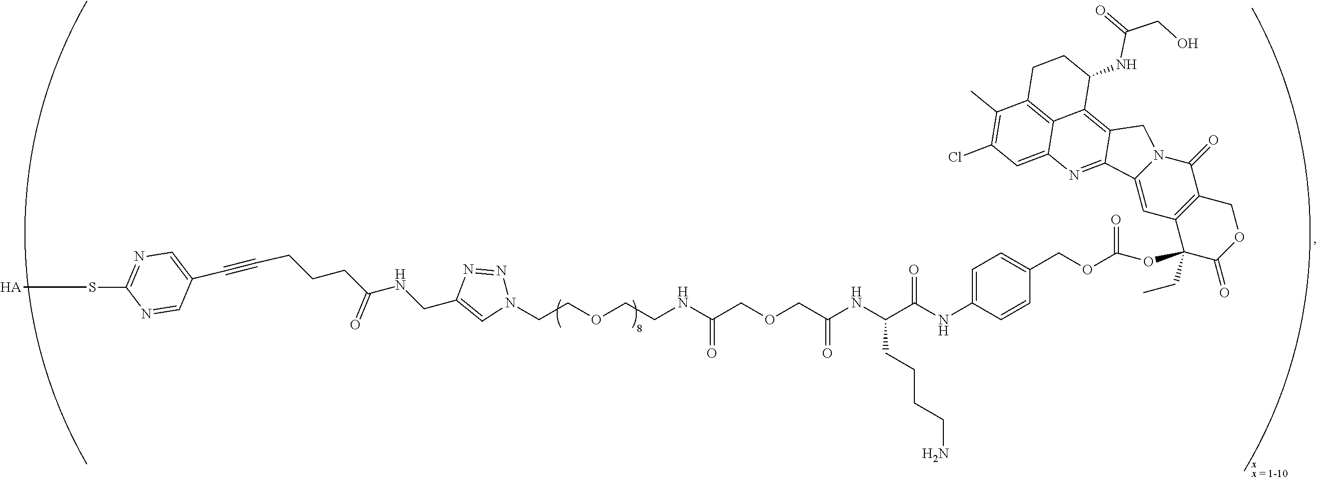

wherein the HA in the antibody-drug conjugate is selected from:
(1) an antibody or antigen-binding fragment thereof comprising a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44;
(2) an antibody or antigen-binding fragment thereof comprising a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44;
(3) an antibody or antigen-binding fragment thereof comprising a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44;
(4) an antibody or antigen-binding fragment thereof comprising a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44; and
(5) an antibody or antigen-binding fragment thereof comprising a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44,

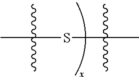 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

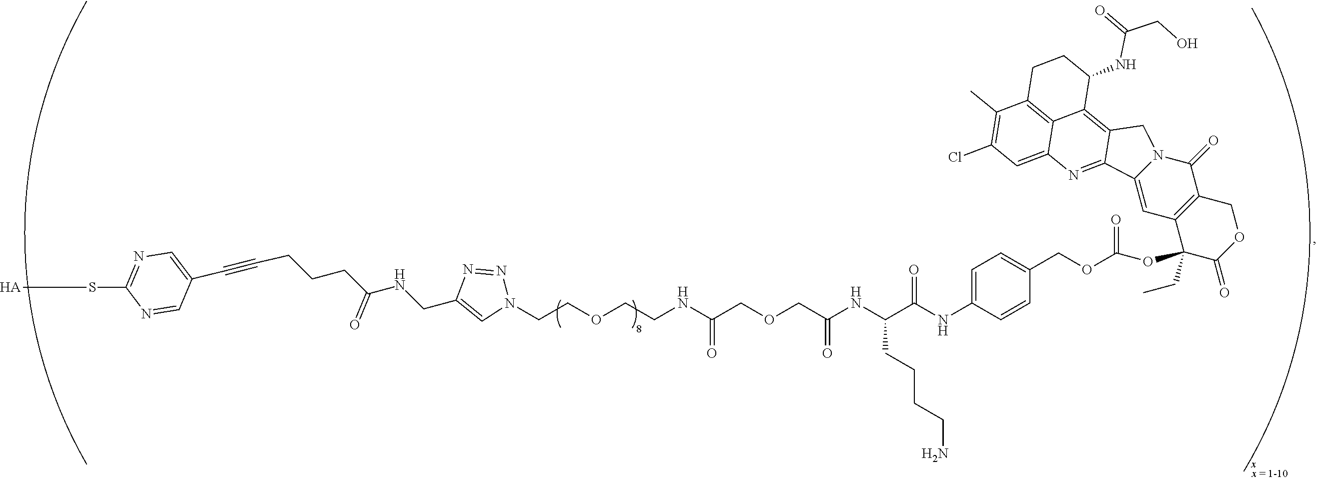

wherein the HA in the antibody-drug conjugate is an antibody or antigen binding fragment comprising: (1) a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44,

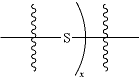 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

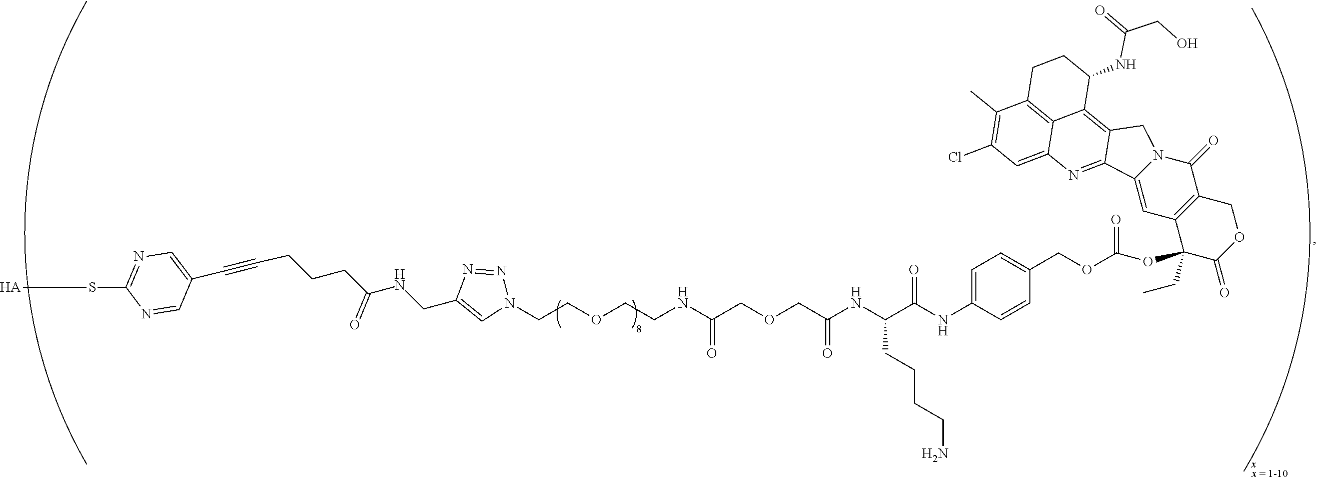

wherein the HA in the antibody-drug conjugate is an antibody or antigen binding fragment comprising: (2) a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44,

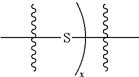 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

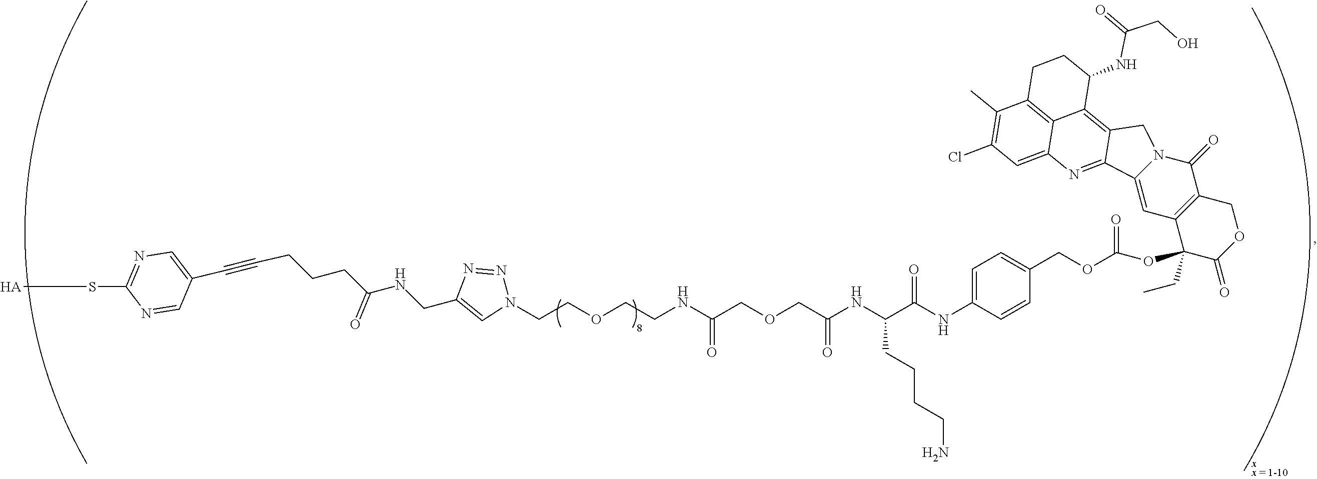

wherein the HA in the antibody-drug conjugate is an antibody or antigen binding fragment comprising: (3) a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44,

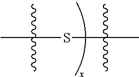 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

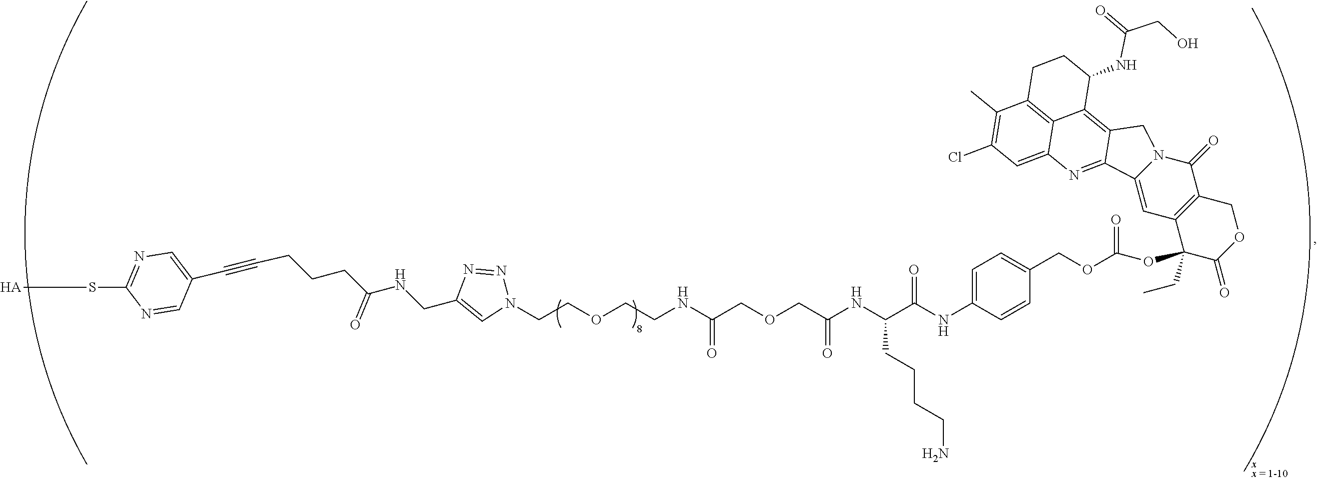

wherein the HA in the antibody-drug conjugate is an antibody or antigen binding fragment comprising: (4) a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44,

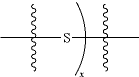 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

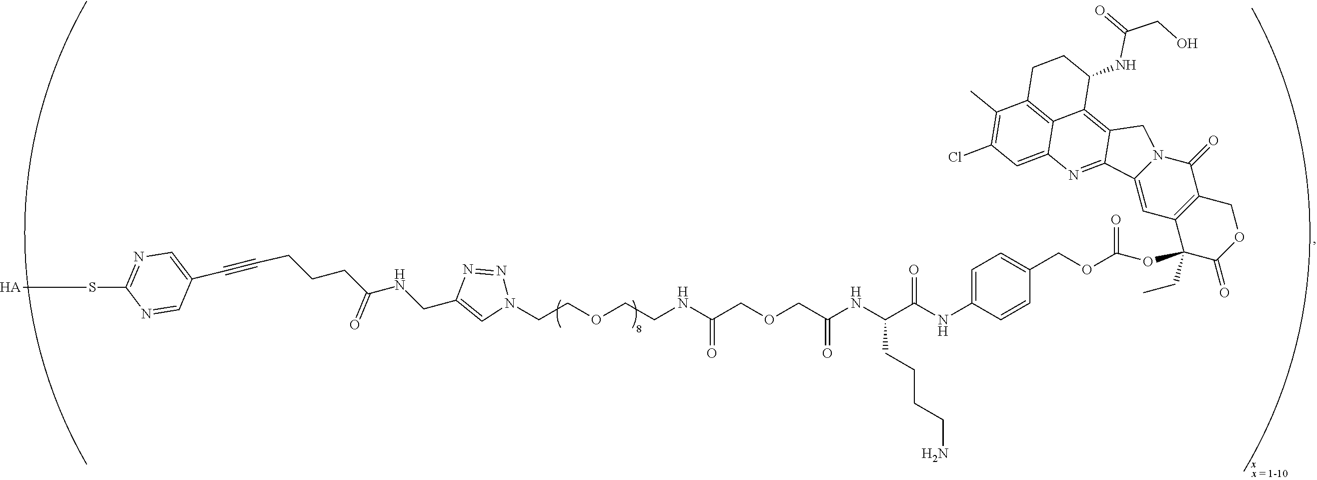

wherein the HA in the antibody-drug conjugate is an antibody or antigen binding fragment comprising: (5) a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44,

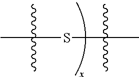 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

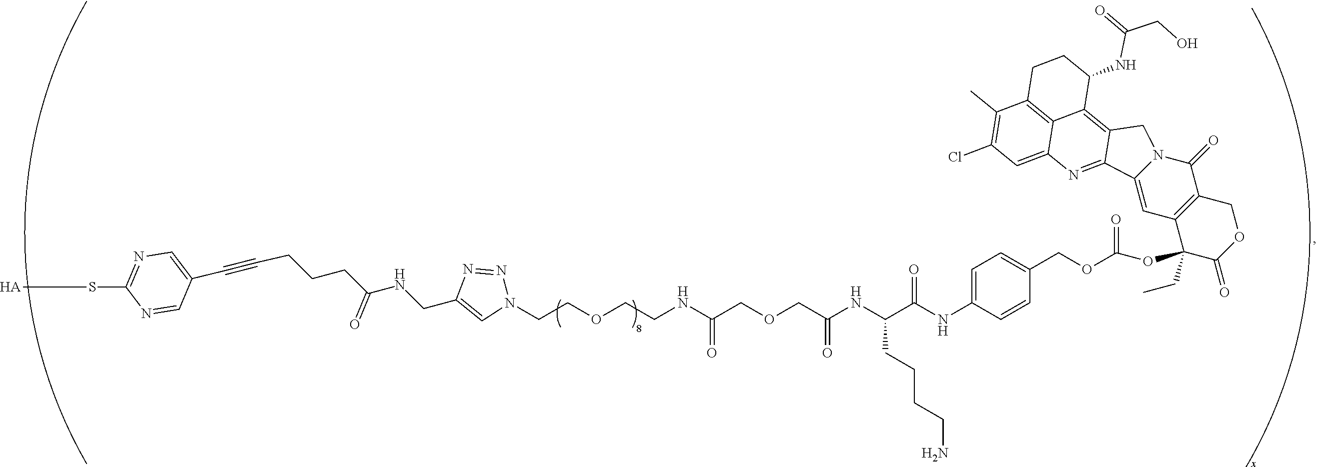

wherein HA in the antibody-drug conjugate is an antibody or antigen-binding fragment thereof comprising: a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 44;
x is 7 to 9,

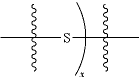 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

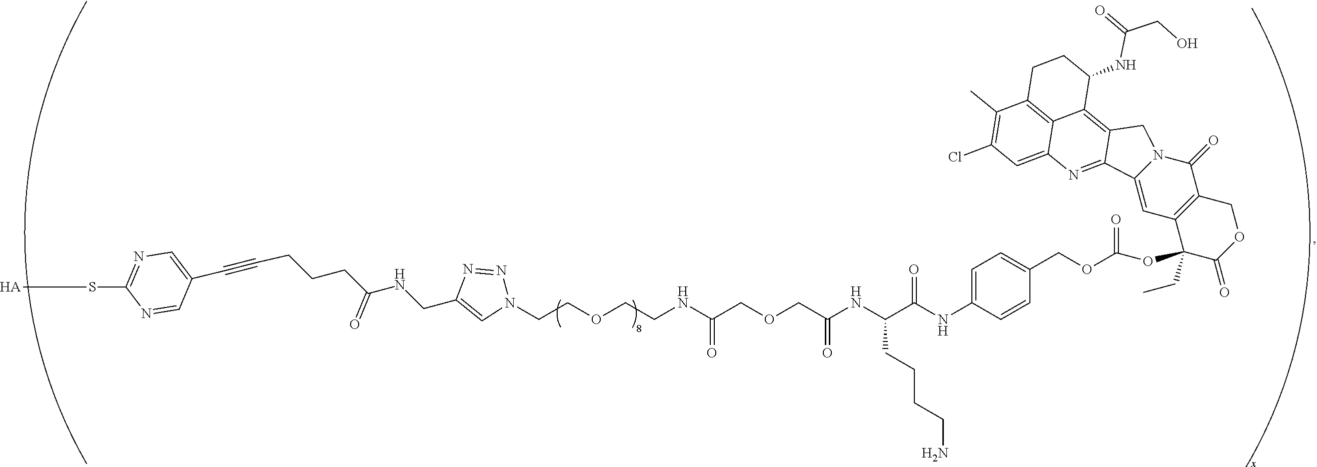

wherein HA in the antibody-drug conjugate is an antibody or antigen-binding fragment thereof comprising: a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 47;
x is 7 to 9,

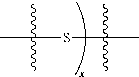 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

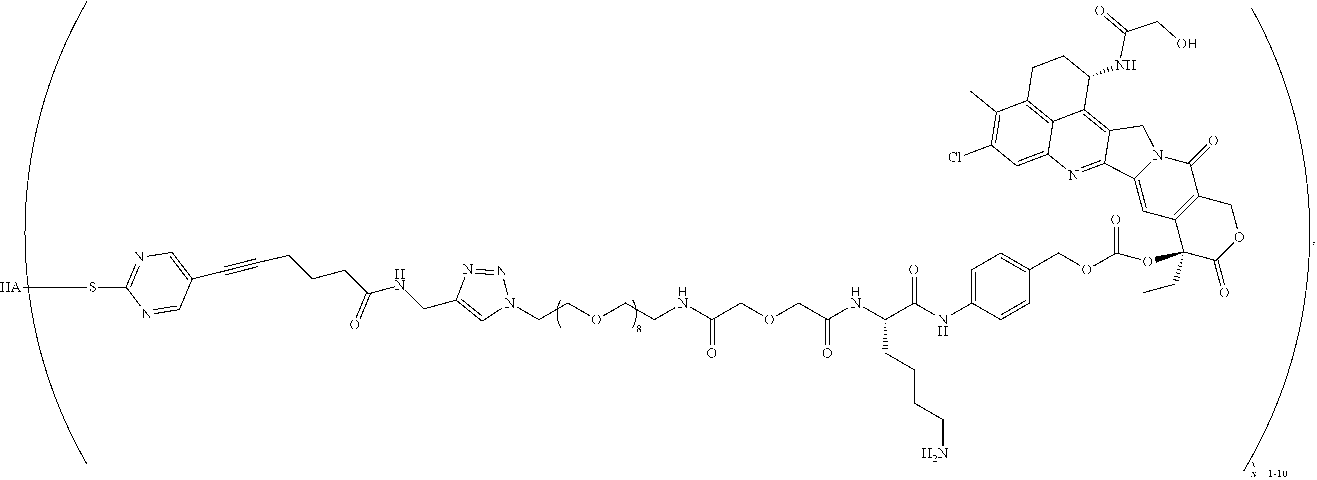

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47;
x is 7 to 8,

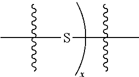 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

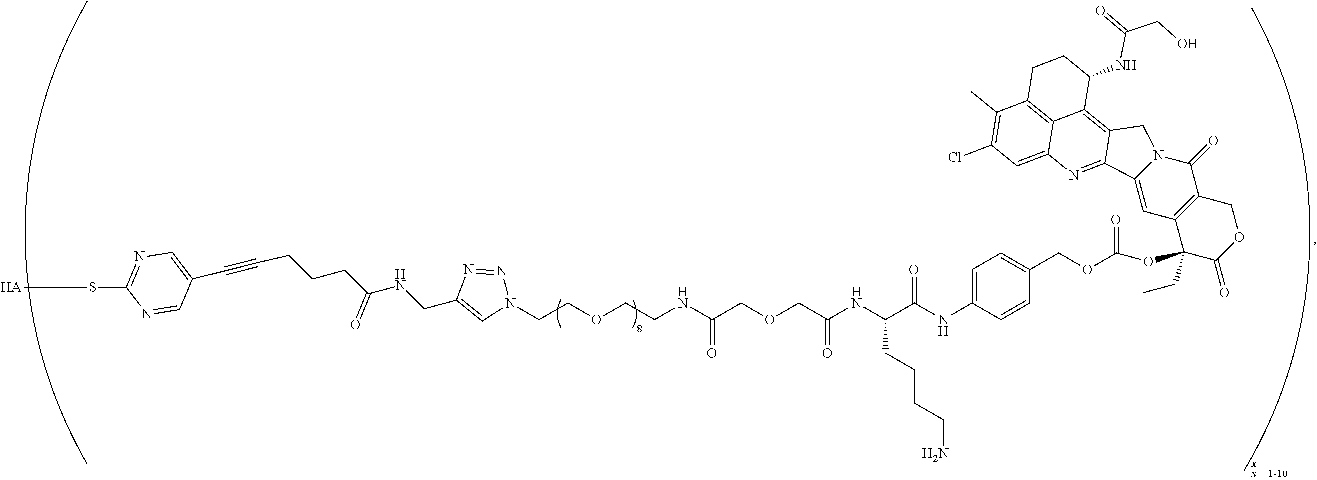

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47;
x is 8 to 9,

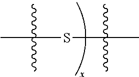 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

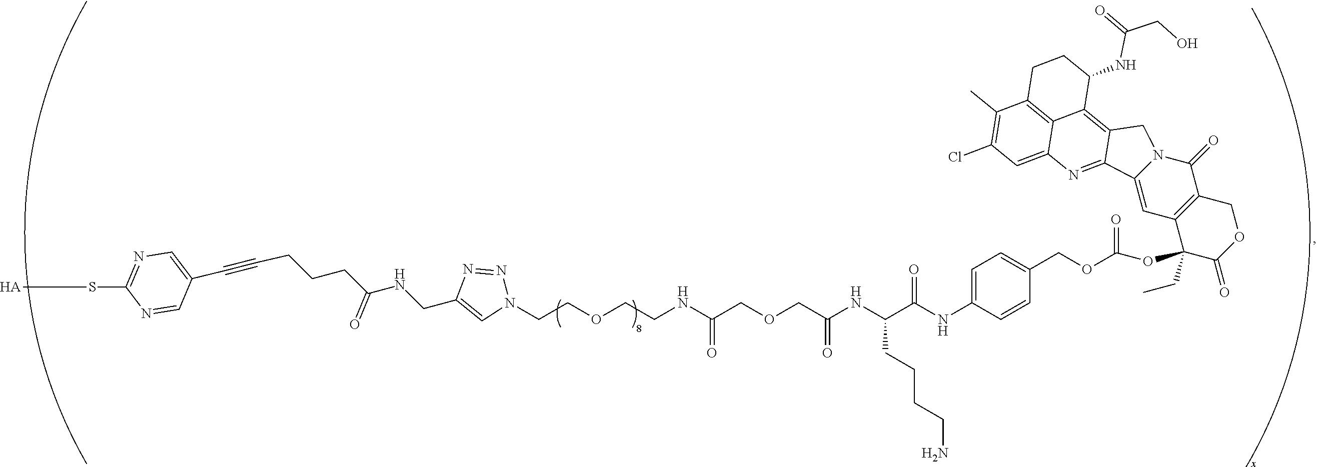

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47;
x is 8,

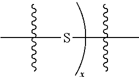 [SMILES: *S*]

represents linkage formed from a sulfhydryl in the antibody or antigen-binding fragment and the linker, and
HA is linked via the sulfhydryls in the antibody or antigen-binding fragment to form the antibody-drug conjugate.

In a further embodiment, the antibody-drug conjugate is:

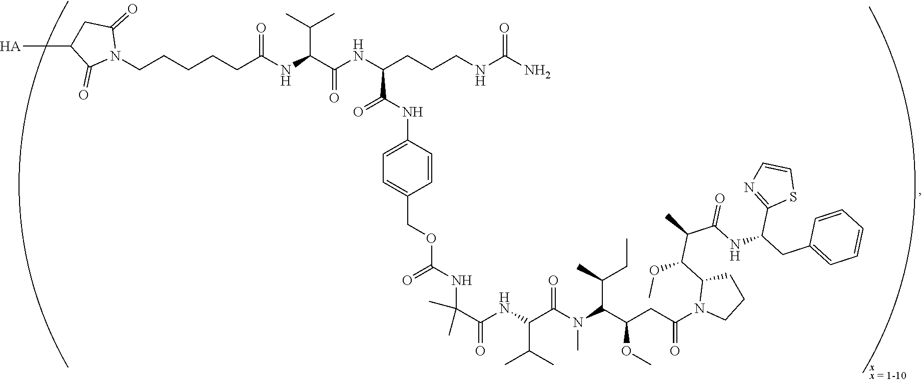

wherein the HA in the antibody-drug conjugate is selected from:
(1) an antibody or antigen-binding fragment thereof comprising a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44;
(2) an antibody or antigen-binding fragment thereof comprising a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44;
(3) an antibody or antigen-binding fragment thereof comprising a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44;
(4) an antibody or antigen-binding fragment thereof comprising a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44; and
(5) an antibody or antigen-binding fragment thereof comprising a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44.

In a further embodiment, the antibody-drug conjugate is:

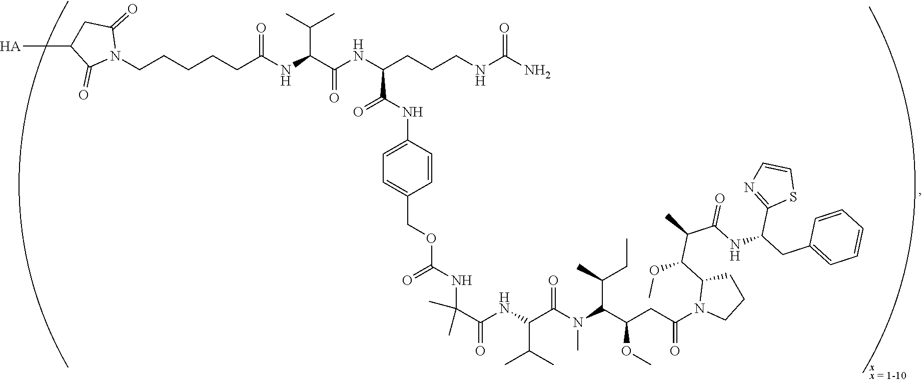

wherein the HA in the antibody-drug conjugate is an antibody or antigen-binding fragment thereof comprising (1) a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44.

In a further embodiment, the antibody-drug conjugate is:

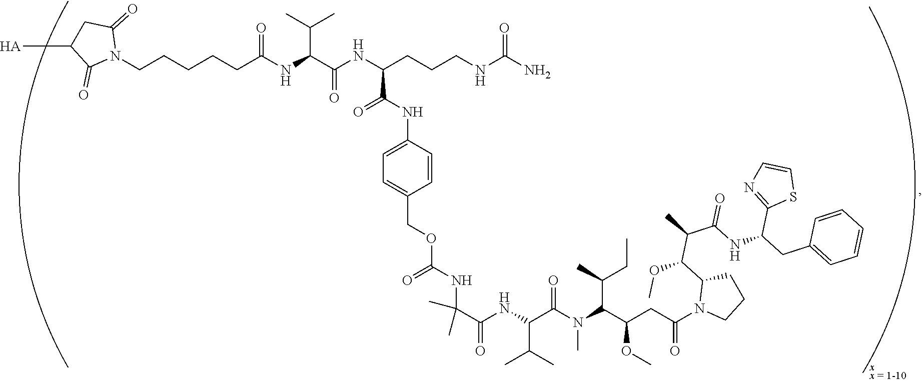

wherein the HA in the antibody-drug conjugate is an antibody or antigen-binding fragment thereof comprising (2) a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44.

In a further embodiment, the antibody-drug conjugate is:

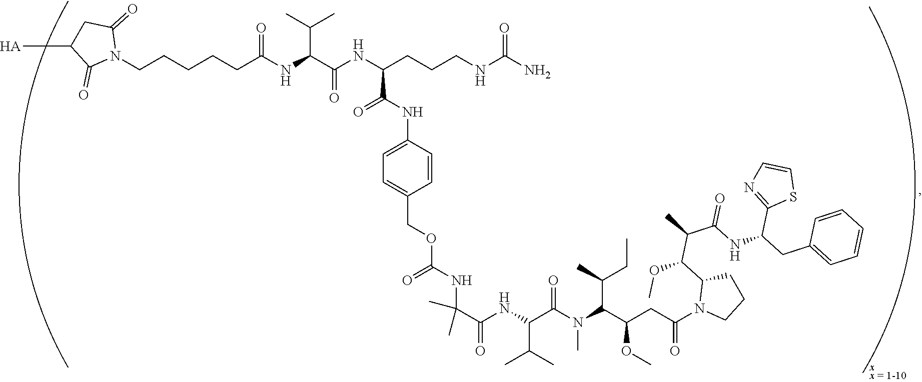

wherein the HA in the antibody-drug conjugate is an antibody or antigen-binding fragment thereof comprising (3) a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44.

In a further embodiment, the antibody-drug conjugate is:

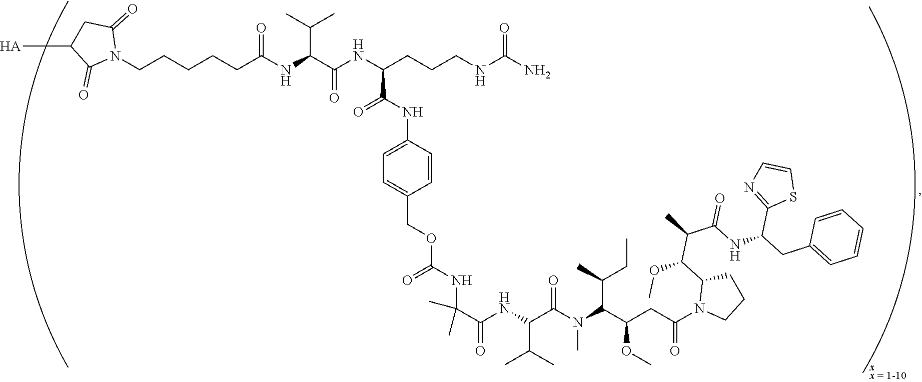

wherein the HA in the antibody-drug conjugate is an antibody or antigen-binding fragment thereof comprising (4) a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44.

In a further embodiment, the antibody-drug conjugate is:

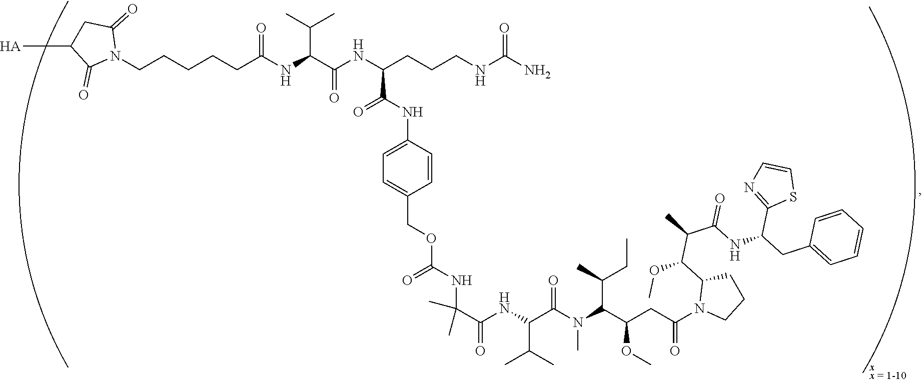

wherein the HA in the antibody-drug conjugate is an antibody or antigen-binding fragment thereof comprising (5) a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44.

In a further embodiment, the antibody-drug conjugate is:

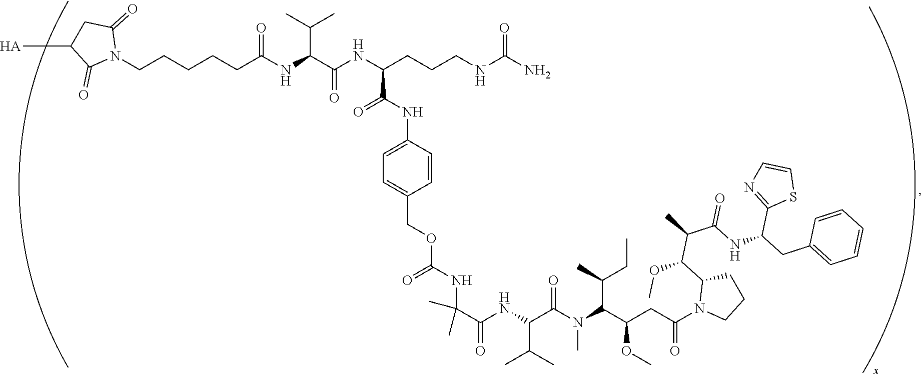

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 2 to 5 or 3 to 4.

In some embodiments, the antibody-drug conjugate (ADC) has an x of 1 to 10, for example, 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10.

In some embodiments, the antibody-drug conjugate (ADC) has an x of 1, 2, 3, 4, 5, 6, 7, 8, 9, or 10.

In some embodiments, the antibody disclosed herein is genetically engineered to comprise one or more cysteine or lysine residue or non-canonical amino acid substitutions of amino acids at defined locations within the antibody.

In some embodiments, the antibody disclosed herein is genetically engineered to comprise one or more cysteine or non-canonical amino acid substitutions of amino acids at defined locations within the antibody. These cysteine residues or non-canonical amino acid residues may then be conjugated to drug-linker via the sulfhydryl group of the cysteine residues or the reactive group of the non-canonical amino acid. Thus, the antibody-drug conjugate of the present invention may comprise one or more substitutions of an amino acid in the heavy chain or light chain of the antibody with a cysteine residue or non-canonical amino acid residue, which is then conjugated to a drug-linker disclosed herein. In particular embodiments, the amino acid positions that may be substituted are selected from positions 152, 153, 171, 172, 173, and 375 of the heavy chain constant domain (numbering according to Eu numbering scheme) and positions 165 and 168 of the light chain constant domain (numbering beginning with amino acid 1 at N-terminus). In particular embodiments, cysteine may be substituted for the amino acid at one or more of the positions 152, 153, 171, 172, 173, and 375 of the heavy chain constant domain (numbering according to Eu numbering scheme) and positions 165 and 168 of the light chain constant domain (numbering beginning with amino acid 1 at N-terminus). In particular embodiments, the antibody-drug conjugate comprises an S375C amino acid substitution that is conjugated to a drug-linker disclosed herein. In particular embodiments, the antibody comprises an S375C amino acid substitution and an E152C amino acid substitution, each conjugated to a drug-linker disclosed herein. In particular embodiments, the antibody comprises an S375C amino acid substitution and an S168C amino acid substitution, each conjugated to a drug-linker disclosed herein. In some embodiments, as explained herein, a composition of the ADC has a DAR value (drug-to-antibody ratio) of 1 to 10, for example: 1 to 2, 1 to 3, 1 to 4, 1 to 5, 1 to 6, 1 to 7, 1 to 8, 1 to 9, 1 to 10, 2 to 3, 2 to 4, 2 to 5, 2 to 6, 2 to 7, 2 to 8, 2 to 9, 2 to 10, 3 to 4, 3 to 5, 3 to 6, 3 to 7, 3 to 8, 3 to 9, 3 to 10, 4 to 5, 4 to 6, 4 to 7, 4 to 8, 4 to 9, 4 to 10, 5 to 6, 5 to 7, 5 to 8, 5 to 9, 5 to 10, 6 to 7, 6 to 8, 6 to 9, 6 to 10, 7 to 8, 7 to 9, 7 to 10, 8 to 9, 8 to 10, or 9 to 10, preferably 3 to 9, for example, 3.0 to 3.5, 3.0 to 4.0, 3.0 to 4.5, 3.0 to 5.0, 3.0 to 5.5, 3.0 to 6.0, 3.5 to 4.0, 3.5 to 4.5, 3.5 to 5.0, 3.5 to 5.5, 3.5 to 6.0, 3.5 to 6.5, 3.5 to 7.0, 3.5 to 7.5, 3.5 to 8.0, 4.0 to 4.5, 4.0 to 5.0, 4.0 to 5.5, 4.0 to 6.0, 4.0 to 6.5, 4.0 to 7.0, 4.0 to 7.5, 4.0 to 8.0, 4.5 to 5.0, 4.5 to 5.5, 4.5 to 6.0, 4.5 to 6.5, 4.5 to 7.0, 4.5 to 7.5, 4.5 to 8.0, 5.0 to 5.5, 5.0 to 6.0, 5.0 to 6.5, 5.0 to 7.0, 5.0 to 7.5, 5.0 to 8.0, 5.5 to 6.0, 5.5 to 6.5, 5.5 to 7.0, 5.5 to 7.5, 5.5 to 8.0, 6.0 to 6.5, 6.0 to 7.0, 6.0 to 7.5, 6.0 to 8.5, 6.5 to 7.0, 6.5 to 7.5, 6.5 to 8.5, 7.0 to 7.5, 7.0 to 9.0 or 7.5 to 9.0.

### Drug-Linker

Those skilled in the art should be understood that the antibody-drug conjugate described in the present application can be prepared in a modularized manner. For example, the “drug-linker” in a free form (which can be understood as M′-L-E-D, wherein M′ is a structural form before covalent linkage of M to the antibody or antigen binding fragment thereof) is obtained, and then the ‘drug-linker’ in the free form is covalently linked to the antibody or antigen binding fragment thereof to obtain the antibody-drug conjugate according to the present application. Correspondingly, M′ in the free form of ‘drug-linker’ is linked to one or more sulfhydryl (—SH), amino (—NH₂) or carboxyl (—COOH) on the antibody or antigen binding fragment thereof through a substitution reaction (for example, removal of —SO₂Me or —Br and the like structures thereon) or through an addition reaction and other manners.

In another aspect, the present invention provides a drug-linker having a structure shown as the formula M′-L-E-D, where
M′ is

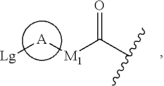

and Lg is a leaving group for a nucleophilic substitution reaction (for example, halogen, methanesulfonyl, fluorophenol, or

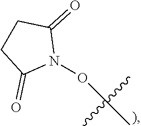 [SMILES: *ON1C(=O)CCC1=O]

or hydroxyl (—OH), sulfhydryl (—SH) or amino (—NH₂); or, Lg forms an unsaturated double bond with an adjacent atom on ring A; ring A is a 5-6 membered aliphatic heterocyclic ring, or a 5-20 membered aromatic ring system, and the aliphatic heterocyclic ring and the aromatic ring system are optionally substituted by one or more groups selected from oxo (═O), halogen, cyano, amino, carboxyl, sulfhydryl and C₁₋₆ alkyl; and M₁ is selected from a single bond, C₁₋₂₀ alkylene, C₂₋₂₀ alkenylene and C₂₋₂₀ alkynylene; and
L, E and D structures are defined according to any one of above antibody-drug conjugate.

In some embodiments, M′ is

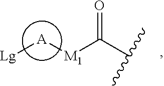

Lg is methanesulfonyl, or Lg and adjacent atoms on the ring A form carbon-carbon double bonds; the ring A is a 5-membered alicyclic heterocyclic ring, a 6-membered heteroaromatic ring, or a polycyclic ring formed by linking more than one 6-membered heteroaromatic ring and a benzene ring through a single bond, and the alicyclic heterocyclic ring is optionally substituted by one or more groups selected from oxo (═O), halogen and C₁₋₄ alkyl; and M₁ is selected from a single bond, C₃₋₁₀ alkylene, C₃₋₁₀ alkenylene and C₃₋₁₀ alkynylene.

In some embodiments, M′ is

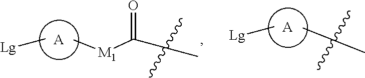

is selected from

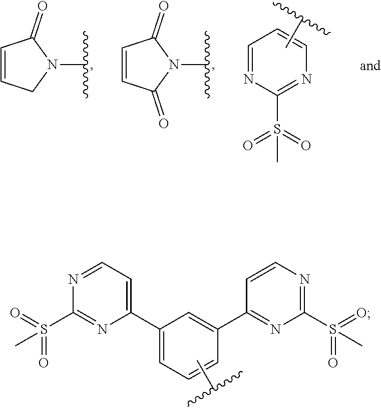

and M₁ is selected from a single bond, C₅₋₈ alkylene, C₅₋₈ alkenylene and C₅₋₈ alkynylene.

In some embodiments, M′ is selected from

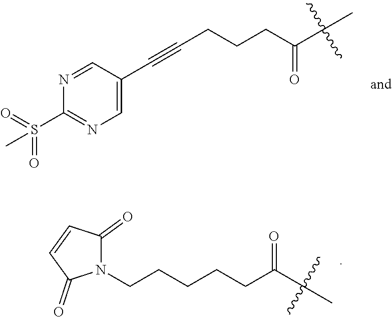 [SMILES: *C(=O)CCCC#Cc1cnc(S(C)(=O)=O)nc1 ; "and" ; *C(=O)CCCCCN1C(=O)C=CC1=O ; "."]

In some embodiments, M′ is

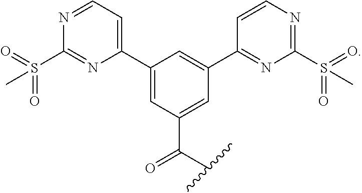 [SMILES: *C(=O)c1cc(-c2ccnc(S(C)(=O)=O)n2)cc(-c2ccnc(S(C)(=O)=O)n2)c1]

In some embodiments, the “drug-linker” in free form is selected from A-01 to A-26, B-01 to B-06, and C-01 shown as follows:

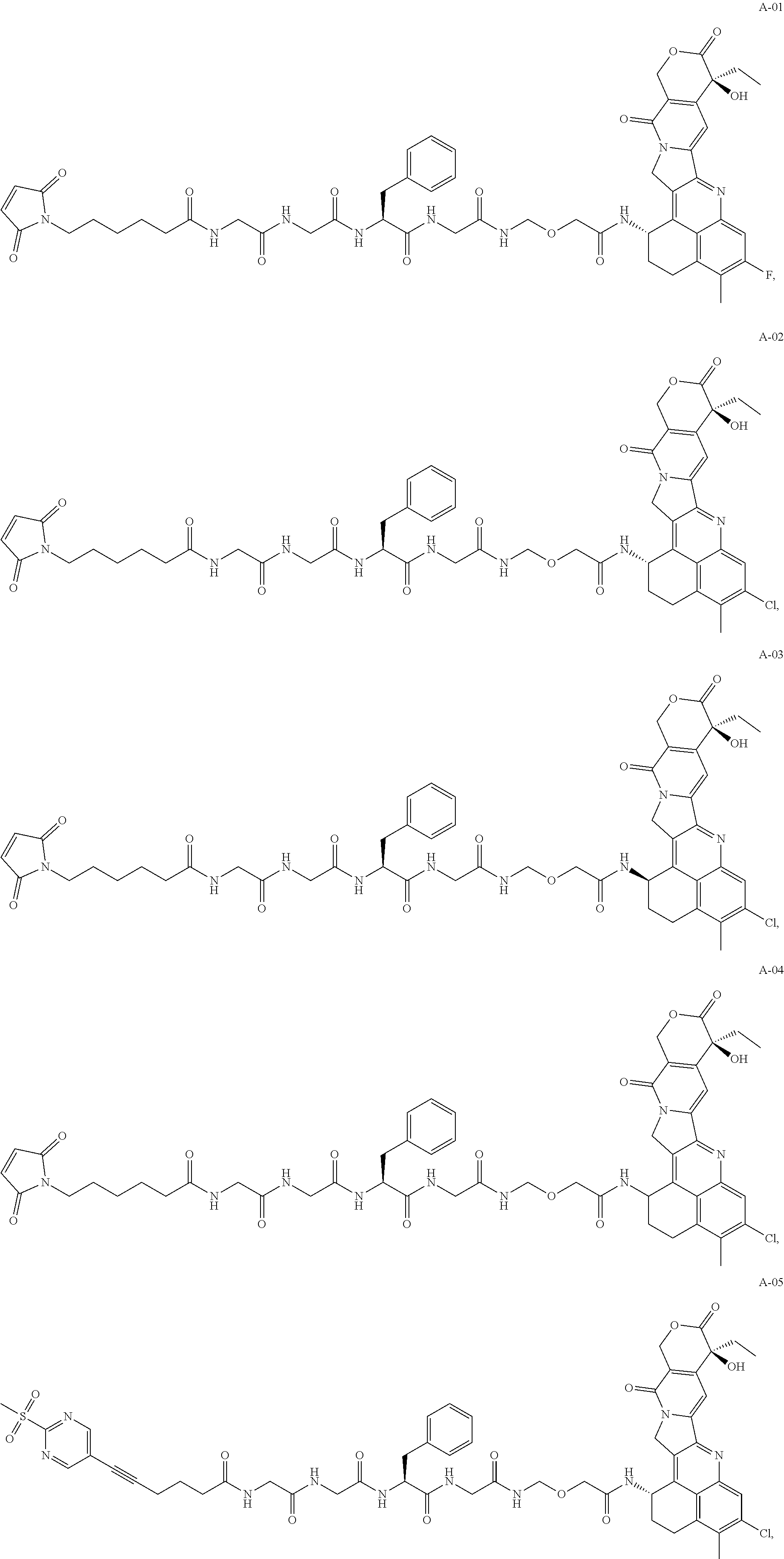

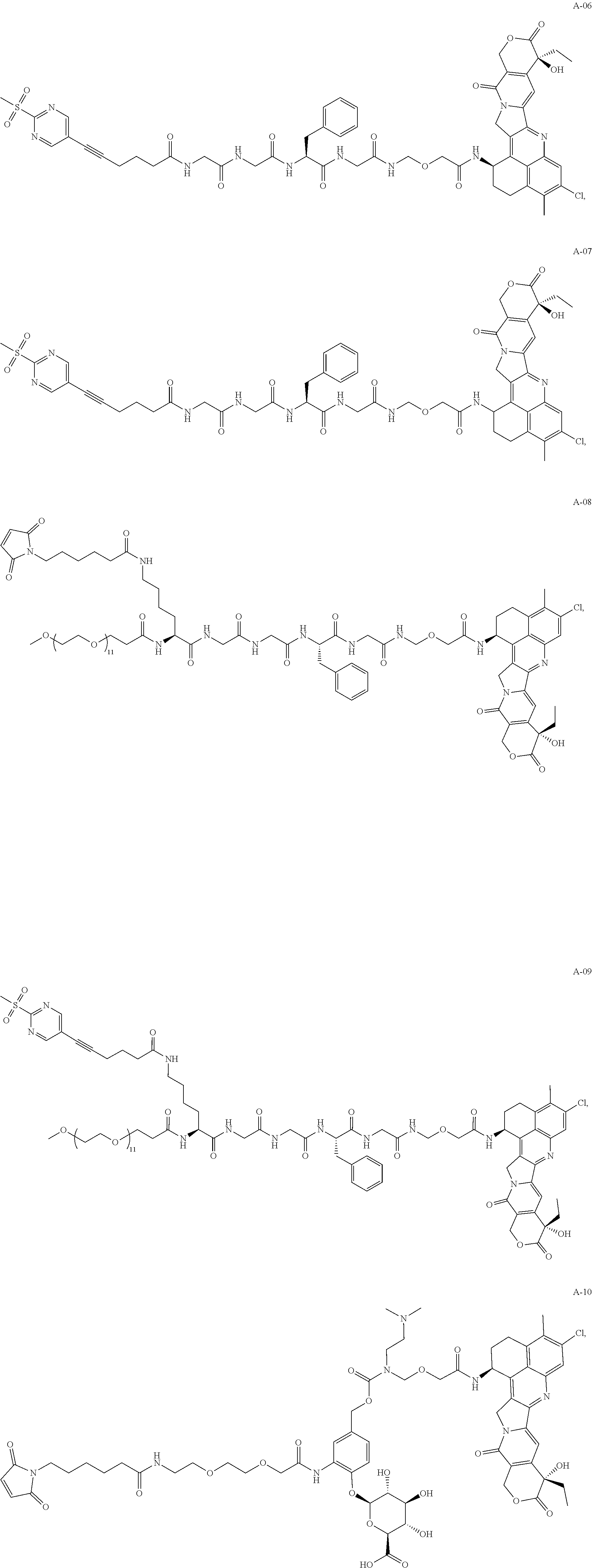

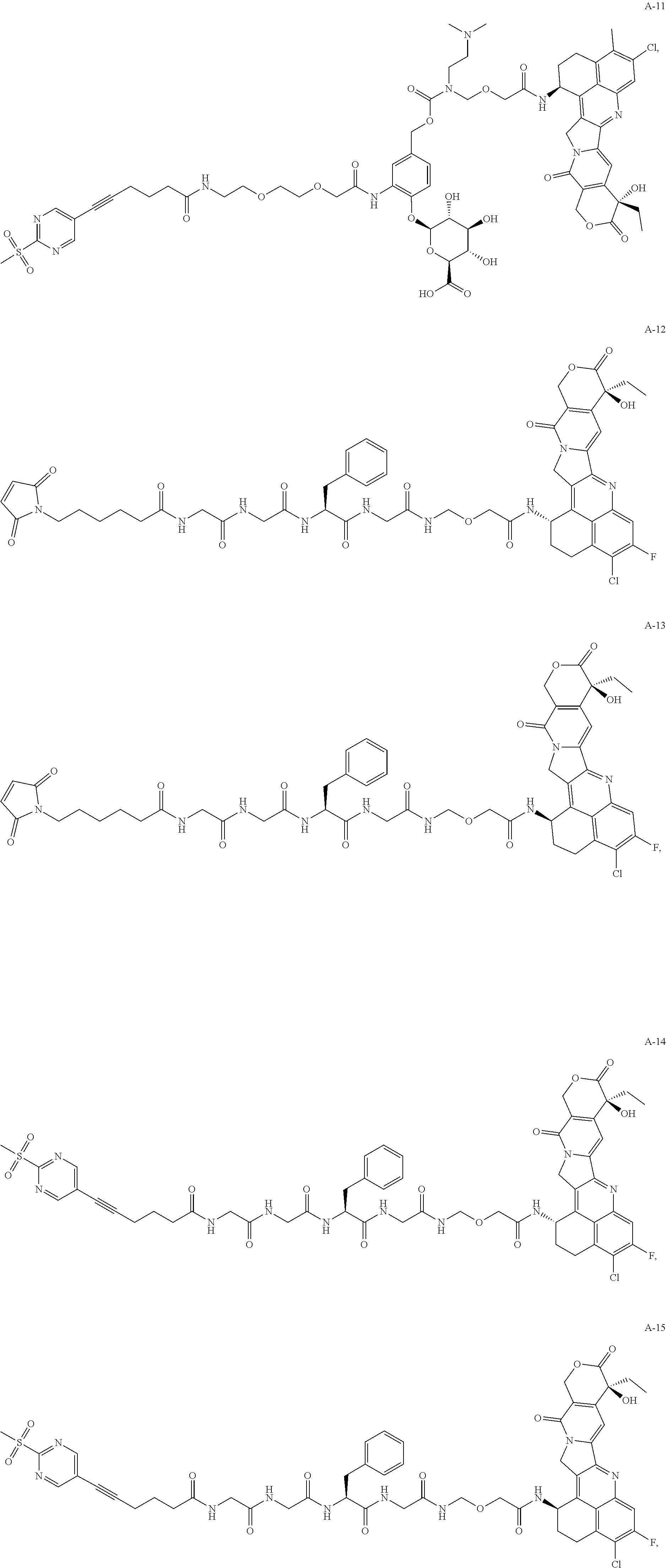

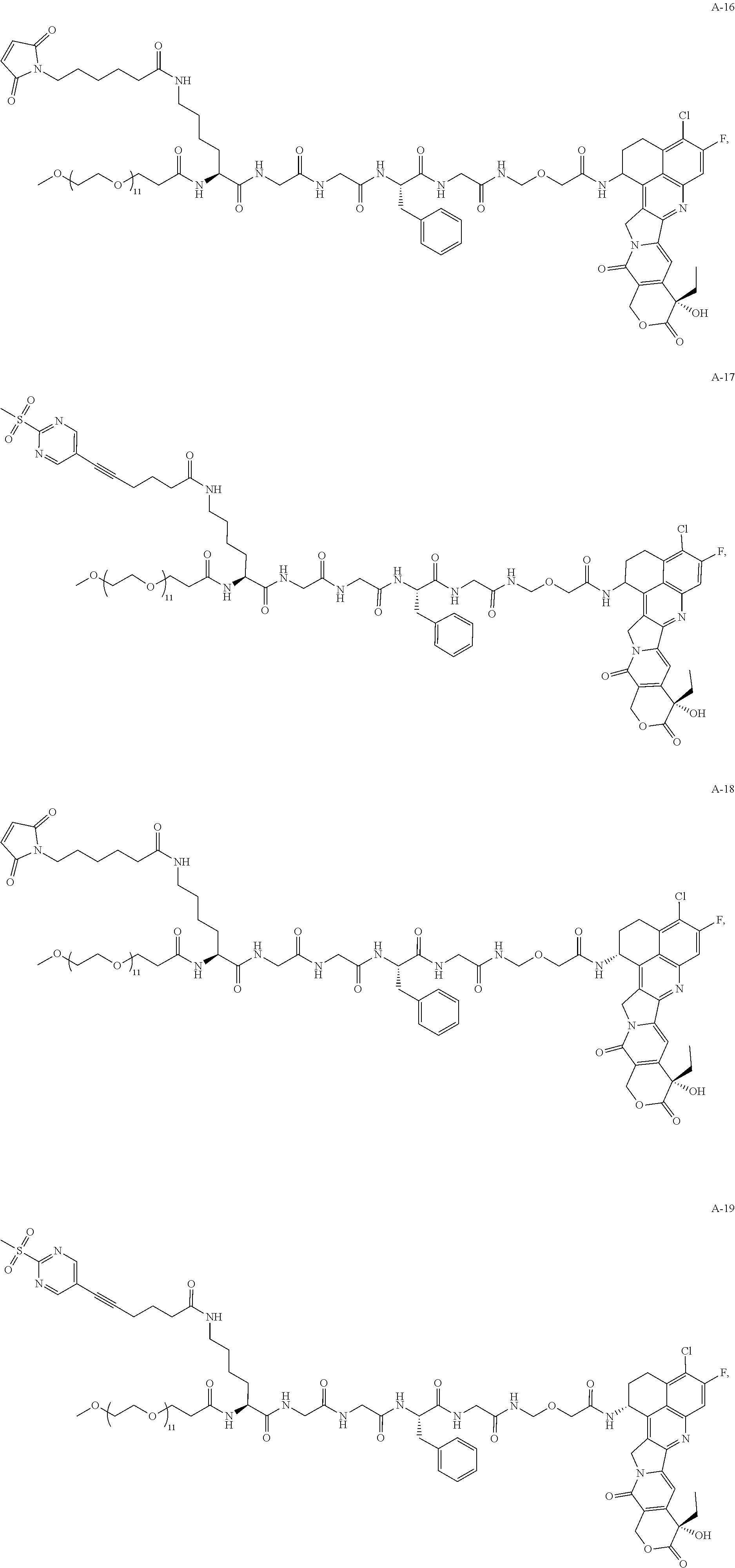

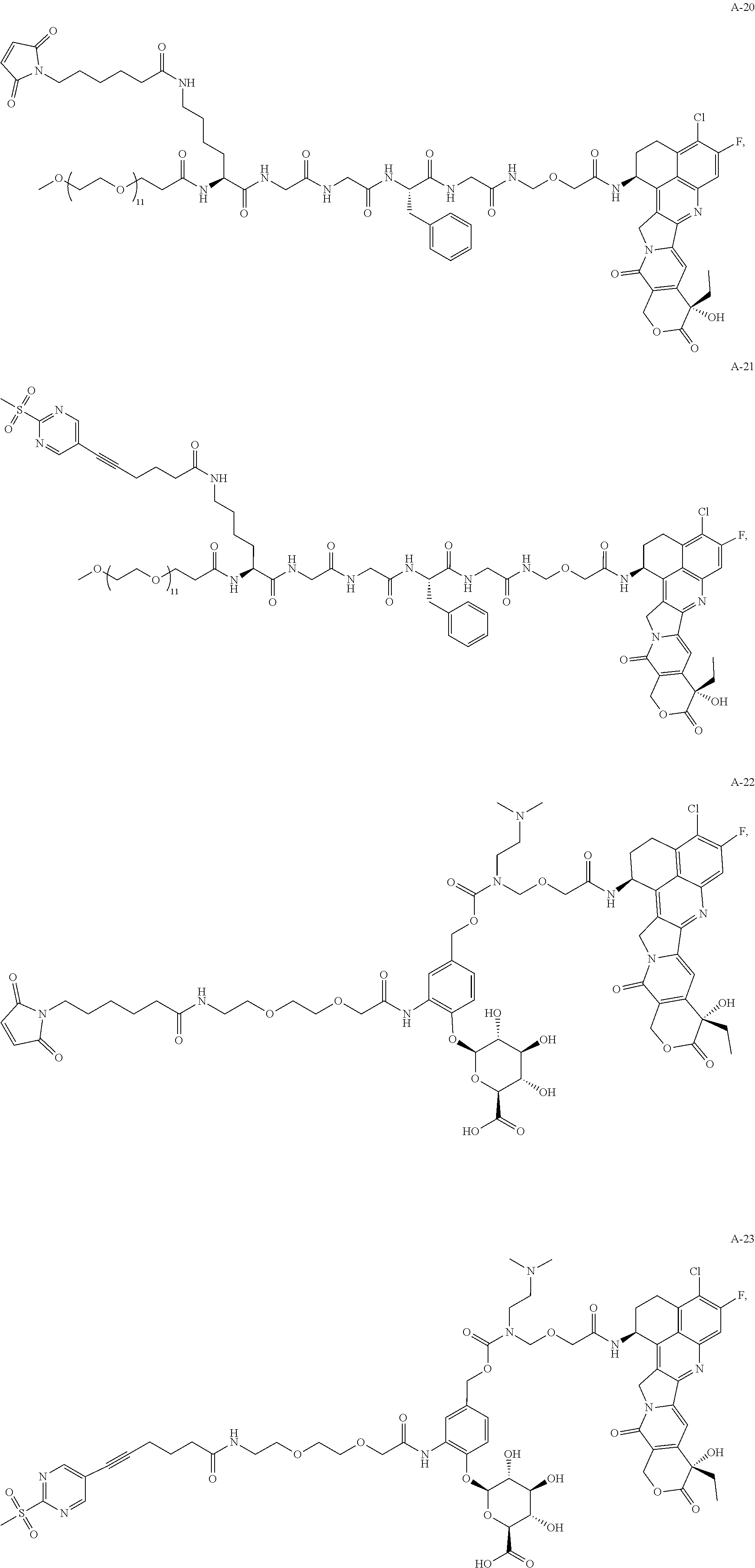

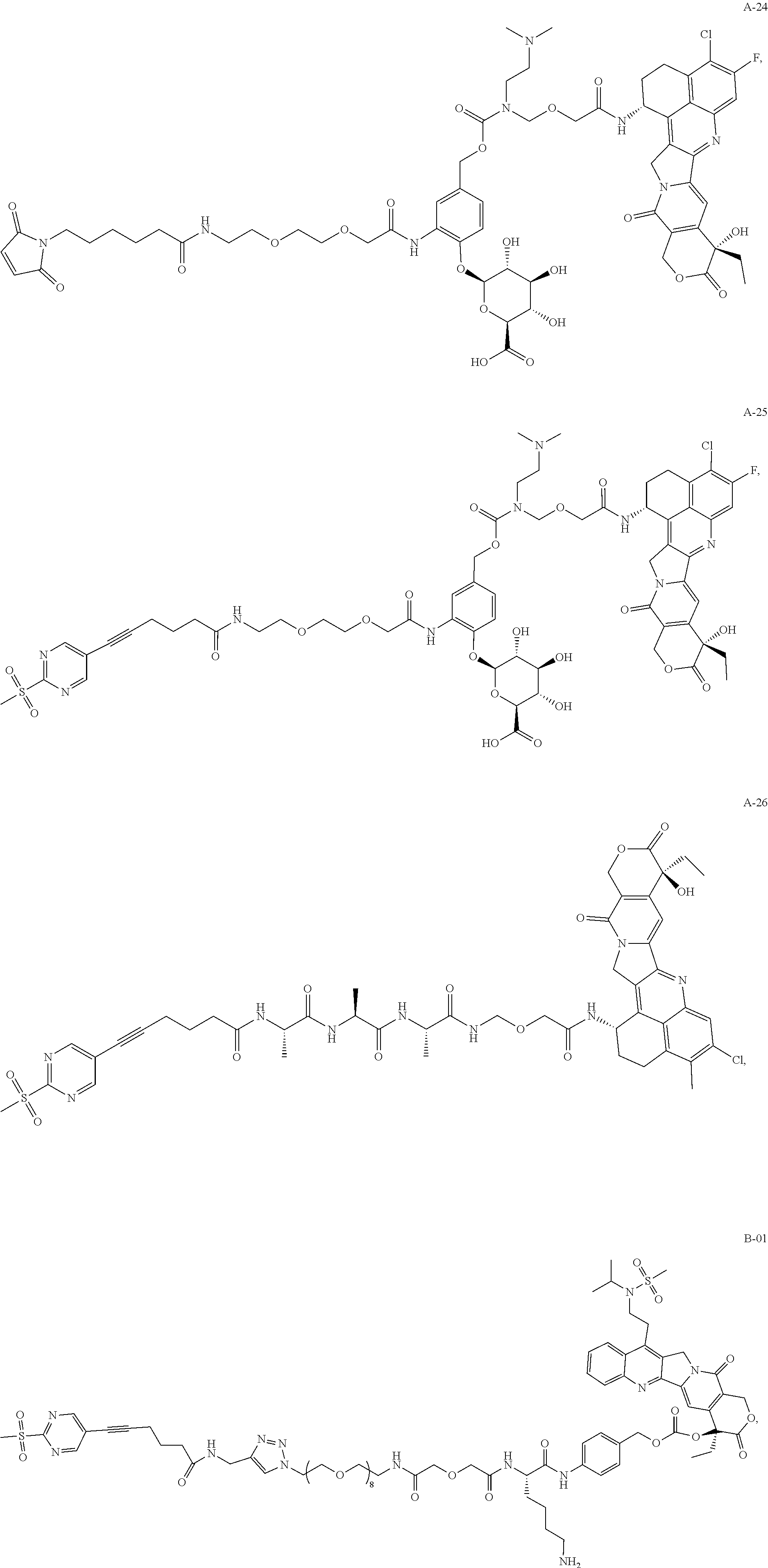

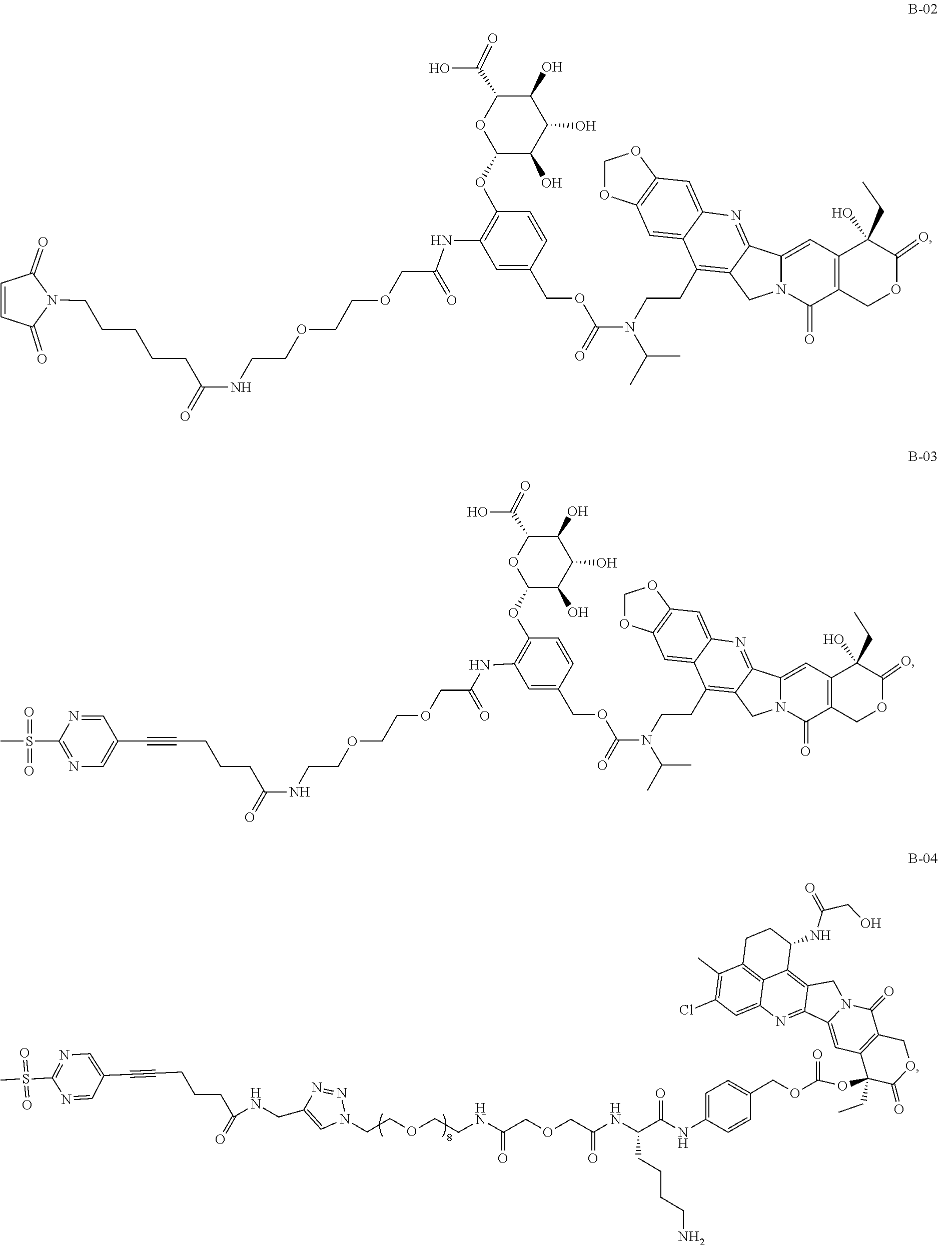

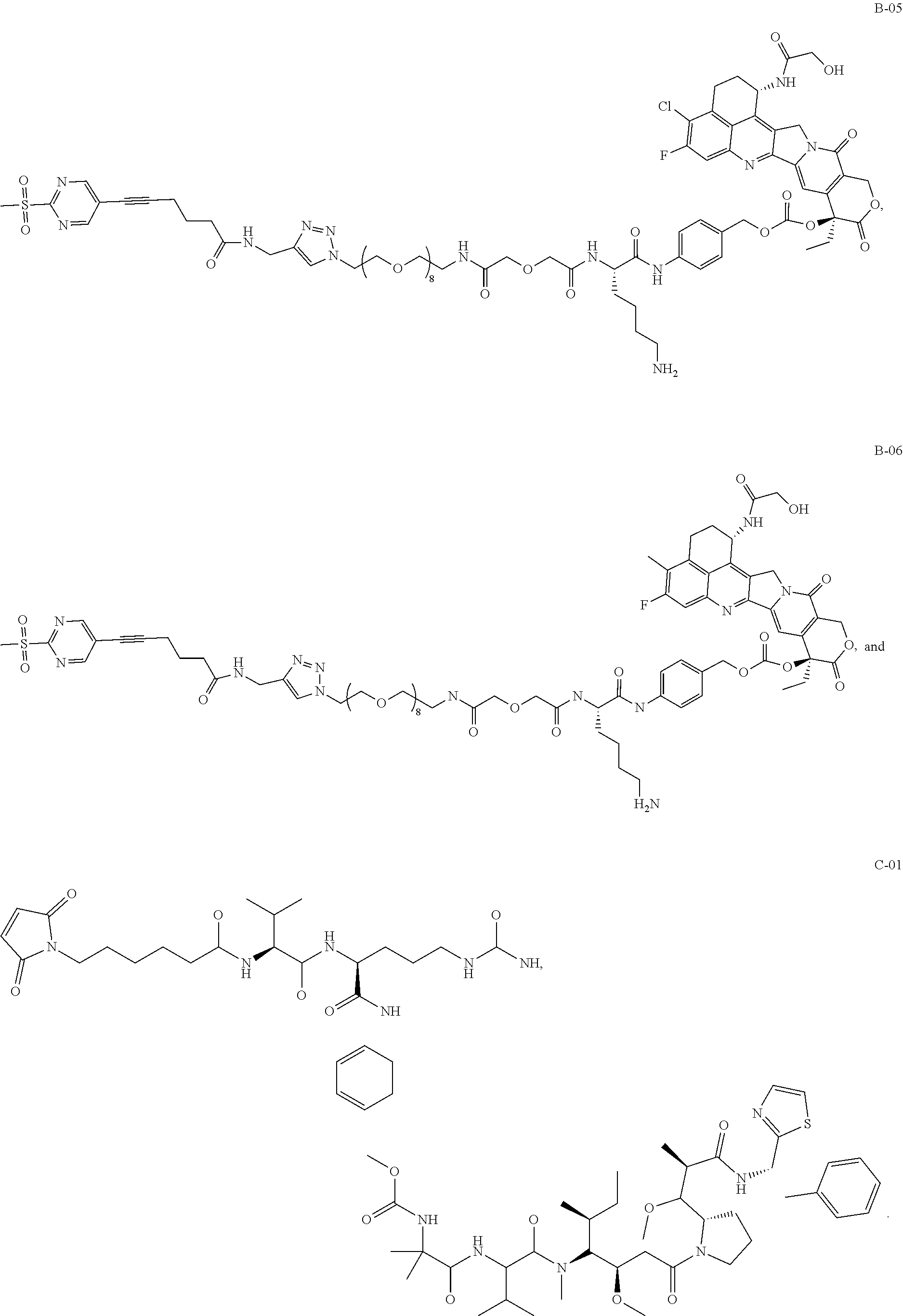

The present invention further provides an ADC comprising an antibody that binds PTK7 conjugated via a cysteine or lysine or a non-canonical amino acid substitution of an amino acid within the antibody residue to a drug-linker selected from the group consisting of A-01, A-02, A-03, A-04, A-05, A-06, A-07, A-08, A-09, A-10,A-12, A13, A-14, A15, A-16, A-17, A-18, A-19, A-20, A-21, A-22, A-23, A-24, A-25, A-26, B-01, B-02, B-03, B-04, B-05, B-06 and C-01.

The present invention further provides an ADC comprising an antibody that binds PTK7 conjugated via a cysteine or a non-canonical amino acid substitution of an amino acid within the antibody residue to a drug-linker selected from the group consisting of A-01, A-02, A-03, A-04, A-05, A-06, A-07, A-08, A-09, A-10, A-12, A13, A-14, A15, A-16, A-17, A-18, A-19, A-20, A-21, A-22, A-23, A-24, A-25, A-26, B-01, B-02, B-03, B-04, B-05, B-06 and C-01.

In a further embodiment of the ADC, the antibody is an antibody or antigen binding fragment selected from the group consisting of:
(1) the following heavy chain variable regions (VH) and/or light chain variable regions (VL), wherein CDRs are defined according to a Chothia numbering system:
(1a) a heavy chain variable region (VH) comprising the following 3 CDRs: CDR-H1 having the amino acid sequence as set forth in SEQ ID NO: 11 or a variant thereof, CDR-H2 having the amino acid sequence as set forth in SEQ ID NO: 12 or a variant thereof, and CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(1b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (1a) and (1b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example replacement, deletion or addition of 1, 2 or 3 amino acids) compared with the sequence from which the variants are derived; preferably, the replacement is conservative replacement;
   or,
(2) the following heavy chain variable region (VH) and/or light chain variable region (VL), wherein the CDRs are defined according to the Kabat numbering system:
(2a) a heavy chain variable region (VH) comprising the following 3 CDRs: CDR-H1 having the amino acid sequence as set forth in SEQ ID NO: 17 or a variant thereof, CDR-H2 having the amino acid sequence as set forth in SEQ ID NO: 18 or 19 or a variant thereof, CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(2b) a heavy chain variable region (VH) comprising the following 3 CDRs: CDR-H1 having the amino acid sequence as set forth in SEQ ID NO: 33 or a variant thereof, CDR-H2 having the amino acid sequence as set forth in SEQ ID NO: 34 or 35 or a variant thereof, and CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (2a) and (2b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement;
   or,
(3) the following heavy chain variable regions (VH) and/or light chain variable regions (VL), wherein the CDRs are defined according to the IMGT numbering system:
(3a) a heavy chain variable region (VH) comprising the following 3 CDRs: CDR-H1 having the amino acid sequence as set forth in SEQ ID NO: 20 or a variant thereof, CDR-H2 having the amino acid sequence as set forth in SEQ ID NO: 21 or a variant thereof, and CDR-H3 having the amino acid sequence as set forth in SEQ ID NO: 22 or a variant thereof; and/or, a light chain variable region (VL) comprising the following 3 CDRs: CDR-L1 having the amino acid sequence as set forth in SEQ ID NO: 23 or a variant thereof, CDR-L2 having the amino acid sequence as set forth in SEQ ID NO: 24 or a variant thereof, and CDR-L3 having the amino acid sequence as set forth in SEQ ID NO: 16 or a variant thereof; or,
(3b) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, and CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 39 or a variant thereof, CDR-L2 having the amino acid sequence as set forth in SEQ ID NO: 40 or a variant thereof, and CDR-L3 having the amino acid sequence as set forth in SEQ ID NO: 32 or a variant thereof;
where the variants in any one of (3a) and (3b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement;
   and,
(4) the following heavy chain variable regions (VH) and/or light chain variable regions (VL), wherein the CDRs are defined according to the AbM numbering system:
(4a) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, and CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(4b) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, and CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (4a) and (4b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids (for example, replacement, deletion or addition of 1, 2 or 3 amino acids) compared with the sequence from which the variants are derived; preferably, the replacement is conservative replacement.

In a further embodiment of the ADC, the antibody or antigen binding fragment is selected from the group consisting of:
(a) a VH having the amino acid sequence as set forth in SEQ ID NO: 1 or a variant thereof, and/or, a VL having the amino acid sequence as set forth in SEQ ID NO: 2 or a variant thereof;
(b) a VH having the amino acid sequence as set forth in SEQ ID NO: 3 or a variant thereof, and/or, a VL having the amino acid sequence as set forth in SEQ ID NO: 4 or a variant thereof;
(c) a VH having the amino acid sequence as set forth in SEQ ID NO: 5 or a variant thereof, and/or, a VL having the amino acid sequence as set forth in SEQ ID NO: 6 or a variant thereof;
(d) a VH having the amino acid sequence as set forth in SEQ ID NO: 7 or a variant thereof, and/or, a VL having the amino acid sequence as set forth in SEQ ID NO: 8 or a variant thereof; and
(e) a VH having the amino acid sequence as set forth in SEQ ID NO: 9 or a variant thereof, and/or, a VL having the amino acid sequence as set forth in SEQ ID NO: 10 or a variant thereof. In a further embodiment of the ADC, the antibody or antigen binding fragment is selected from the group consisting of
(1) an antibody or antigen-binding fragment thereof comprising a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44;
(2) an antibody or antigen-binding fragment thereof comprising a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44;
(3) an antibody or antigen-binding fragment thereof comprising a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44;
(4) an antibody or antigen-binding fragment thereof comprising a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44; and
(5) an antibody or antigen-binding fragment thereof comprising a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44.

In a further embodiment of the ADC, the antibody or antigen binding fragment comprises (1) a VH having the amino acid sequence as set forth in SEQ ID NO: 1, and a VL having the amino acid sequence as set forth in SEQ ID NO: 2, for example an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44.

In a further embodiment of the ADC, the antibody or antigen binding fragment comprises (2) a VH having the amino acid sequence as set forth in SEQ ID NO: 3, and a VL having the amino acid sequence as set forth in SEQ ID NO: 4, for example an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44.

In a further embodiment of the ADC, the antibody or antigen binding fragment comprises (3) a VH having the amino acid sequence as set forth in SEQ ID NO: 5, and a VL having the amino acid sequence as set forth in SEQ ID NO: 6, for example an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44.

In a further embodiment of the ADC, the antibody or antigen binding fragment comprises (4) a VH having the amino acid sequence as set forth in SEQ ID NO: 7, and a VL having the amino acid sequence as set forth in SEQ ID NO: 8, for example an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44.

In a further embodiment of the ADC, the antibody or antigen binding fragment comprises (5) a VH having the amino acid sequence as set forth in SEQ ID NO: 9, and a VL having the amino acid sequence as set forth in SEQ ID NO: 10, for example an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44. In a further embodiment, the antibody comprises a heavy chain (HC) having the amino acid sequence as set forth in SEQ ID NO: 46, and a light chain (LC) having the amino acid sequence as set forth in SEQ ID NO: 47.

### Antibody Preparation

The antibodies described herein can be prepared by various methods known in the art, for example, by genetic engineering and recombination techniques. For example, DNA molecules encoding the heavy and light chain genes of the antibodies of the present invention are obtained by chemical synthesis or PCR amplification. The resulting DNA molecule is inserted into an expression vector, which is then transfected into a host cell. Then, the transfected host cells are cultured under specific conditions, and express the antibody of the present invention.

### Conjugation

In another aspect, the present application provides a method for conjugating the drug-linkers described herein to the antibodies described herein to make the antibody-drug conjugates (ADCs) described herein.

In certain embodiments, the antibody described herein is conjugated to a drug-linker described herein via conjugation to a lysine in the antibody.

In certain embodiments, the antibody described herein is conjugated to a drug-linker described herein via conjugation to a cysteine in the antibody. In certain embodiments, the cysteines are from reduced intrachain disulfide bonds in the antibody. In certain embodiments, the cysteines are from reduced interchain disulfide bonds in the antibody.

In some embodiments, the antibody is conjugated to a drug-linker via conjugation to reduced interchain disulfide bonds in the antibody. For example, an IgG1 antibody consists of four polypeptide chains, two heavy chains comprising VH, CH1 and Fc (e.g., hinge, CH2 and CH3) domains, and two light chains comprising VL and CL domains, connected by interchain cysteine disulfide (—S—S—) bonds (e.g., two heavy chain-light chain interchain disulfide bonds and two hinge heavy chain-heavy chain interchain disulfide bonds). In certain embodiments, when these disulfide bonds are broken under reducing conditions, eight (8) reactive cysteine sulfhydryl moieties are produced. In certain embodiments, each of the eight reactive cysteine sulfhydryl moieties is a point of attachment for a linker-drug, such that a maximum of eight (x=8) drug-linkers may be attached to the reduced antibody. In certain embodiments, any one of the four disulfide bonds is broken under reducing conditions, two (2) reactive cysteine sulfhydryl moieties are produced. In further embodiments, each of the two reactive cysteine sulfhydryl moieties is a point of attachment for a linker-drug, such that two (x=2) linker-drugs may be attached to the reduced antibody. In certain embodiments, any two of the four disulfide bonds are broken under reducing conditions, four (4) reactive cysteine sulfhydryl moieties are produced. In further embodiments, each of the four reactive cysteine sulfhydryl moieties is a point of attachment for a linker-drug, such that four (x=4) linker-drugs may be attached to the reduced antibody. In certain embodiments, any three of the four disulfide bonds are broken under reducing conditions, six (6) reactive cysteine sulfhydryl moieties are produced. In further embodiments, each of the six reactive cysteine sulfhydryl moieties is a point of attachment for a linker-drug, such that six (x=6) linker-drugs may be attached to the reduced antibody.

In some embodiments, the interchain disulfide bond is between two cysteine residues, which are broken under reducing conditions, resulting in two reactive cysteine sulfhydryl moieties. In further embodiments, the interchain disulfide bridge in the antibody is between a heavy chain and a light chain, such as between C220 of a heavy chain and C214 of a kappa light chain according to the EU numbering, or between C220 of a heavy chain according to the EU numbering and C214 of a lambda light chain according to the Kabat numbering. Additionally, or alternatively, the interchain disulfide bridge in the antibody is between two heavy chains, such as between C226 and/or C229 of a first heavy chain and C226 and/or C229 of a second heavy chain according to the EU numbering. In some embodiments, the cysteine residues are in the hinge region of the antibody. In some embodiments, the cysteine residue is at any one or more of positions 220, 226, or 229 in the heavy chain according to EU numbering (also referred to herein as C220, C226 or C229, respectively). In some embodiments, the cysteine residue is at position 214 in the light chain according to EU and/or Kabat numbering (also referred to herein as C214, such as position 214 in the kappa light chain according to EU and Kabat numbering or position 214 in the lambda light chain according to the Kabat numbering). In one embodiment, the cysteine residues are at each of positions 220, 226, and 229 in the heavy chain according to the EU numbering and position 214 in the light chain, according to EU or Kabat numbering. In one embodiment, the cysteine residues are at each of positions 220, 226, and 229 in the heavy chain according to the EU numbering and position 214 in the kappa light chain, according to EU and Kabat numbering. In one embodiment, the cysteine residues are at each of positions 220, 226, and 229 in the heavy chain according to the EU numbering and position 214 in the lambda light chain, according to Kabat numbering. In one embodiment, the cysteine residues are at any one or more of the following positions:
(i) any one, or any two, or any three of positions 220, 226, and 229 in a first heavy chain according to the EU numbering;
(ii) any one, or any two, or any three of positions 220, 226, and 229 in a second heavy chain according to the EU numbering;
(iii) position 214 in a first light chain according to the Kabat numbering; and/or
(iv) position 214 in a second light chain according to the Kabat numbering.

As used herein, C220, C226, and C229 refer to amino acid residues (cysteine, Cys, C) of an immunoglobulin identified according to the EU numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGHGnber.html.

As used herein, the cysteine residue at position 214 of kappa light chain refer to amino acid residues (cysteine, Cys, C) of an immunoglobulin identified according to the Kabat numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGKCnber.html.

As used herein, the cysteine residue at position 214 of lambda light chain refer to amino acid residues (cysteine, Cys, C) of an immunoglobulin identified according to the Kabat numbering. As it would be understood by one of skill in the art, such numberings accordingly represent amino acid residues of a polypeptide aligned to those identified in an immunoglobulin, such as the one shown in www.imgt.org/IMGTScientificChart/Numbering/Hu_IGLCnber.html.

In certain embodiments, the antibodies described herein comprise four interchain disulfide bonds in the hinge region which may be reduced, thereby breaking the bond, and revealing a reactive sulfhydryl moiety that may be conjugated with a maleimide moiety on a drug-linker, such as the maleimide moiety on drug-linkers described herein.

In certain embodiments, the antibodies described herein comprise lysine residues wherein the reactive amine side chains of lysine residues may be conjugated with a drug-linker, such as a maleimide moiety on drug-linkers described herein.

In one embodiment, the present disclosure provides a method of making an ADC described herein, comprising the steps of:
a) providing a solution comprising the antibody;
b) contacting the solution of a) with a reducing agent;
c) contacting the solution of b) with a solution comprising a drug-linker or a salt thereof, as described herein,
to make the ADC.

In one embodiment, the reducing agent is tris(2 carboxyethyl)phosphine (TCEP).

### Compositions

In another aspect, the present application provides a composition of antibody-drug conjugates (ADCs) as described herein. Such composition may comprise a plurality of ADCs as described herein, wherein each ADC comprises a drug-linker as described herein, wherein x is independently 1, 2, 3, 4, 5, 6, 7, 8, 9 or 10. In other words, each antibody molecule in the composition may be conjugated to 1, 2, 3, 4, 5, 6, 7, 8, 9 or 10 drug-linkers. Therefore, a composition may be characterized by a “drug-to-antibody” ratio (DAR) ranging from about 1 to about 10. Methods to determine DAR are well known to the skilled person and include methods using Reverse Phase Chromatography, or HPLC-MS.

For example, in any embodiment, an ADC composition described herein has a DAR of about 1 to about 10 or any subrange there between, for example: about 1 to 2, about 1 to 3, about 1 to 4, about 1 to 5, about 1 to 6, about 1 to 7, about 1 to 8, about 1 to 9, about 1 to 10, about 2 to 3, about 2 to 4, about 2 to 5, about 2 to 6, about 2 to 7, about 2 to 8, about 2 to 9, about 2 to 10, about 3 to 4, about 3 to 5, about 3 to 6, about 3 to 7, about 3 to 8, about 3 to 9, about 3 to 10, about 4 to 5, about 4 to 6, about 4 to 7, about 4 to 8, about 4 to 9, about 4 to 10, about 5 to 6, about 5 to 7, about 5 to 8, about 5 to 9, about 5 to 10, about 6 to 7, about 6 to 8, about 6 to 9, about 6 to 10, about 7 to 8, about 7 to 9, about 7 to 10, about 8 to 9, about 8 to 10, or about 9 to 10.

In certain embodiments, an ADC composition described herein has a DAR of about 3 to 9, for example, about 3.0 to 3.5, about 3.0 to 4.0, about 3.0 to 4.5, about 3.0 to 5.0, about 3.0 to 5.5, about 3.0 to 6.0, about 3.5 to 4.0, about 3.5 to 4.5, about 3.5 to 5.0, about 3.5 to 5.5, about 3.5 to 6.0, about 3.5 to 6.5, about 3.5 to 7.0, about 3.5 to 7.5, about 3.5 to 8.0, about 4.0 to 4.5, about 4.0 to 5.0, about 4.0 to 5.5, about 4.0 to 6.0, about 4.0 to 6.5, about 4.0 to 7.0, about 4.0 to 7.5, about 4.0 to 8.0, about 4.5 to 5.0, about 4.5 to 5.5, about 4.5 to 6.0, about 4.5 to 6.5, about 4.5 to 7.0, about 4.5 to 7.5, about 4.5 to 8.0, about 5.0 to 5.5, about 5.0 to 6.0, about 5.0 to 6.5, about 5.0 to 7.0, about 5.0 to 7.5, about 5.0 to 8.0, about 5.5 to 6.0, about 5.5 to 6.5, about 5.5 to 7.0, about 5.5 to 7.5, about 5.5 to 8.0, about 6.0 to 6.5, about 6.0 to 7.0, about 6.0 to 7.5, about 6.0 to 8.5, about 6.5 to 7.0, about 6.5 to 7.5, about 6.5 to 8.5, about 7.0 to 7.5, about 7.0 to 9.0 or about 7.5 to 9.0.

### Pharmaceutical Compositions

In another aspect, the present application provides a pharmaceutical composition, which comprises the antibody-drug conjugate (ADC) described in any one of the foregoing embodiments and optionally the drug-linker described in any one of the foregoing embodiments, and one or more pharmaceutically acceptable auxiliaries.

In one embodiment, the pharmaceutical compositions provided herein comprises an antibody-drug conjugate (ADC) and one or more pharmaceutically acceptable auxiliaries, wherein the ADC is:

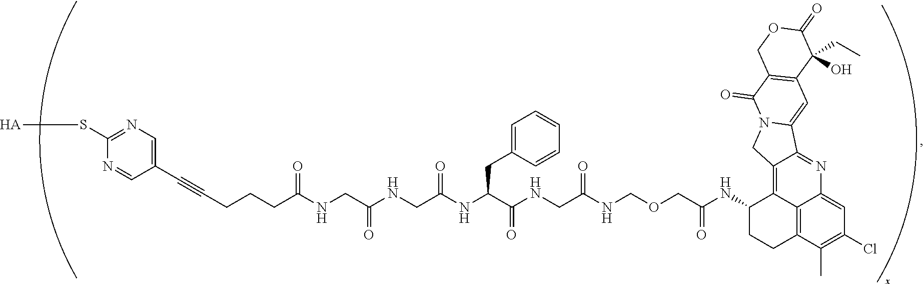

HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 8 to 9.

In one embodiment, the pharmaceutical compositions provided herein comprises an antibody-drug conjugate (ADC) and one or more pharmaceutically acceptable auxiliaries, wherein the ADC is:

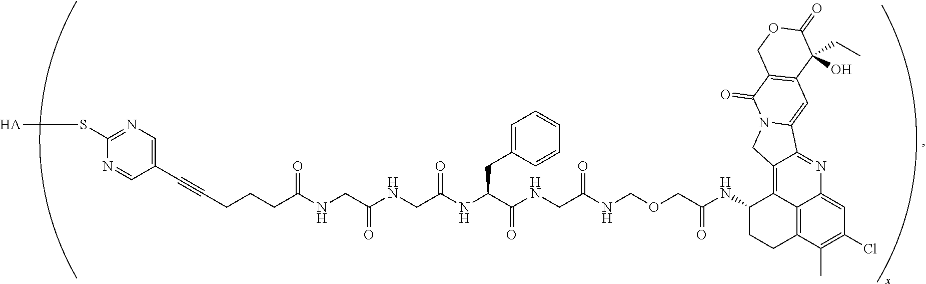

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 8.

In one embodiment, the pharmaceutical compositions provided herein comprises an antibody-drug conjugate (ADC) and one or more pharmaceutically acceptable auxiliaries, wherein the ADC is:

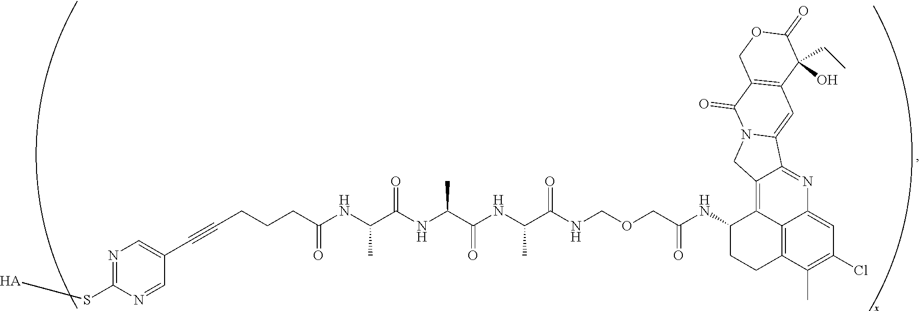

HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 7 to 8.

In one embodiment, the pharmaceutical compositions provided herein comprises an antibody-drug conjugate (ADC) and one or more pharmaceutically acceptable auxiliaries, wherein the ADC is:

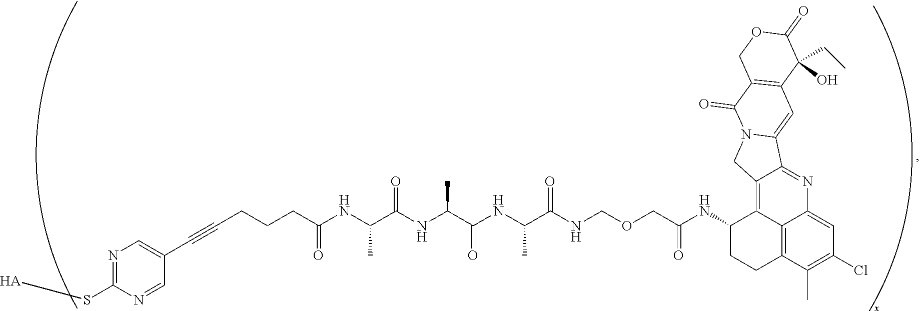

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 8.

In one embodiment, the pharmaceutical compositions provided herein comprises an antibody-drug conjugate (ADC) and one or more pharmaceutically acceptable auxiliaries, wherein the ADC is:

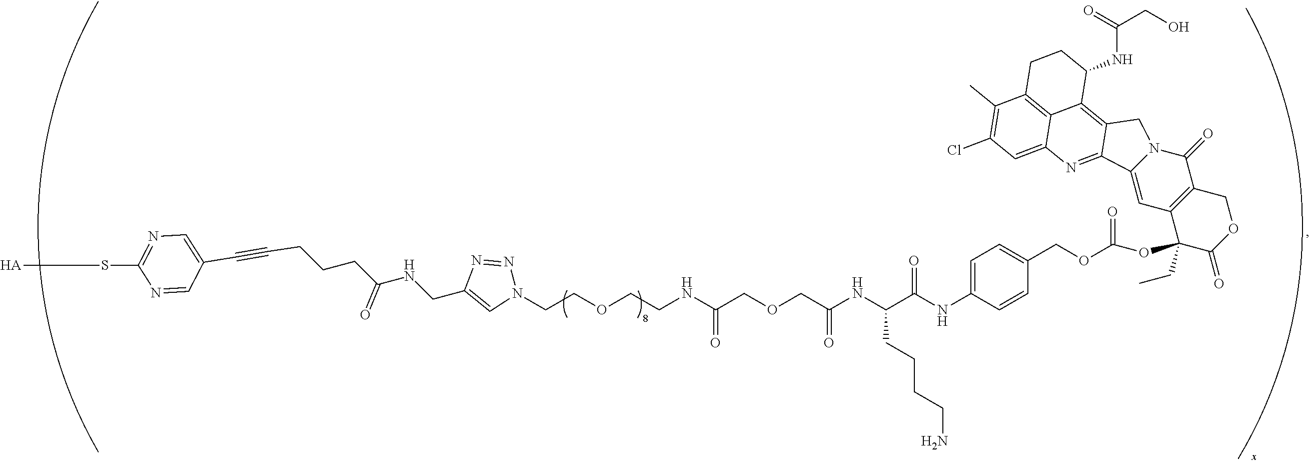

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 7 to 8.

In one embodiment, the pharmaceutical compositions provided herein comprises an antibody-drug conjugate (ADC) and one or more pharmaceutically acceptable auxiliaries, wherein the ADC is:

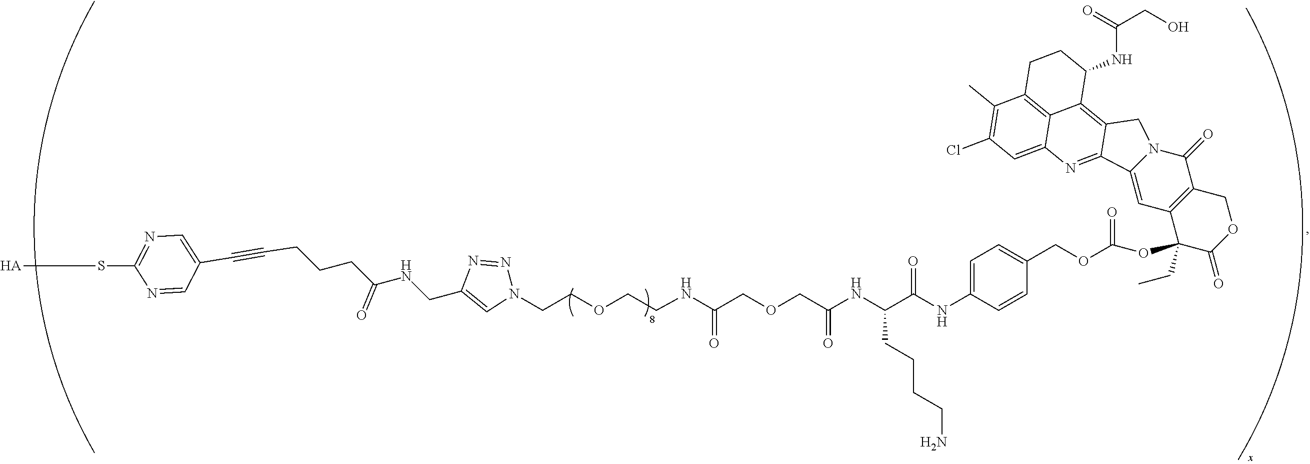

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 8.

In one embodiment, the pharmaceutical compositions provided herein comprises an antibody-drug conjugate (ADC) and one or more pharmaceutically acceptable auxiliaries, wherein the ADC is:
In one embodiment, the pharmaceutical compositions provided herein comprises an antibody-drug conjugate (ADC) and one or more pharmaceutically acceptable auxiliaries, wherein the ADC is:

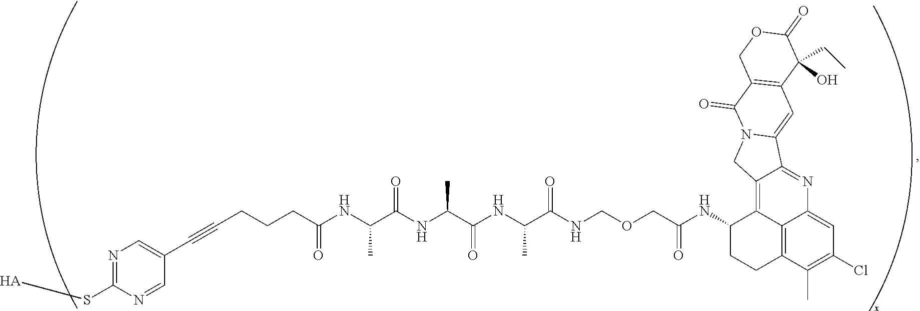

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 8 to 9.

In one embodiment, the pharmaceutical compositions provided herein comprises an antibody-drug conjugate (ADC) and one or more pharmaceutically acceptable auxiliaries, wherein the ADC is:

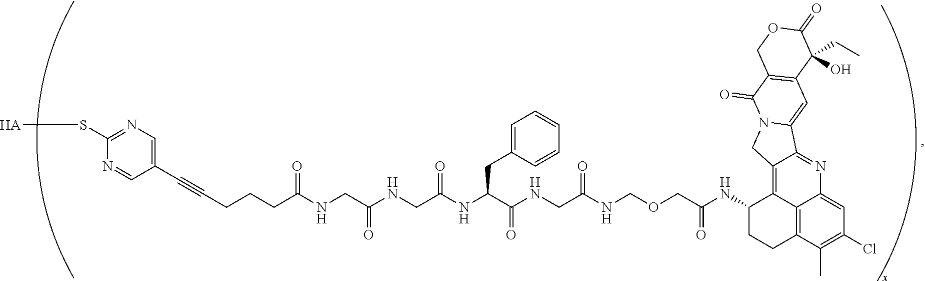

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 8.

In one embodiment, the pharmaceutical compositions provided herein comprises an antibody-drug conjugate (ADC) and one or more pharmaceutically acceptable auxiliaries, wherein the ADC is:

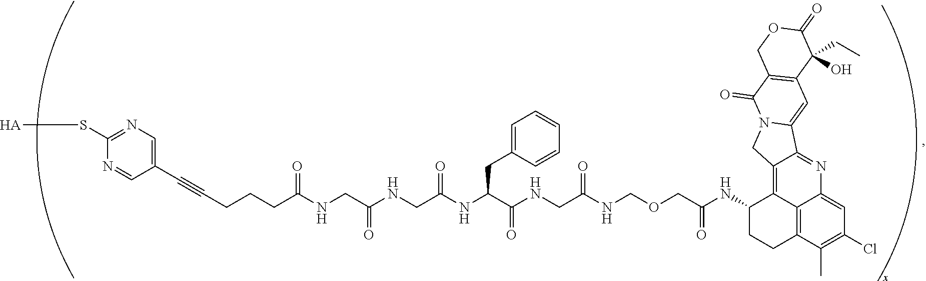

wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; and x is 8 to 9.

Pharmaceutically acceptable auxiliaries include, for example, a pharmaceutically acceptable carrier and/or excipient. Pharmaceutically acceptable auxiliaries further includes salts and solvates.

The ADC described herein is typically formulated with a pharmaceutically acceptable parenteral vehicle to form a unit injectable form for parenteral application, such as bolus injection, intravenous injection, intratumoral injection, and the like. Optionally, the antibody-drug conjugate having the desired purity is mixed with a pharmaceutically acceptable diluents, carriers, excipients or stabilizers in the form of lyophilizate or solution (Remington's Pharmaceutical Sciences (1980) 16^{th} edition, Osol, A. Ed.). The antibody-drug conjugate described herein, or the pharmaceutical composition comprising the antibody-drug conjugate, can be administered via any route appropriate for the individual to be treated.

The ADC and pharmaceutical compositions described herein can be formulated into any dosage form known in the medical field, for example, tablets, pills, suspensions, emulsions, solutions, gels, capsules, powders, granules, elixirs, lozenges, suppositories, injections (including injections, sterile powders for injections and concentrated solutions for injections), inhalants, sprays, etc. The preferred dosage form depends on the intended mode of administration and therapeutic use. Pharmaceutical compositions of the invention should be sterile and stable under the conditions of manufacture and storage. A preferred dosage form is injection. Such injections can be sterile injectable solutions. For example, sterile injectable solutions can be prepared by: The necessary dose of the antibody of the present invention is incorporated in an appropriate solvent, and optionally, other desired ingredients (including but not limited to, pH adjusters, surfactants, adjuvants, ionic strength enhancers, etc. osmotic agent, preservative, diluent, or any combination thereof), followed by filter sterilization. In addition, sterile injectable solutions can be prepared as sterile lyophilized powder (e.g., by vacuum drying or freeze-drying) for ease of storage and use. Such sterile lyophilized powder can be dispersed in a suitable carrier, such as sterile pyrogen-free water, before use.

Furthermore, the ADCs described herein may be presented in pharmaceutical compositions in unit dosage form for ease of administration by any suitable method known in the art, including but not limited to, oral, oral, sublingual, ophthalmic, topical, parenteral, rectal, intrathecal, intracytoplasmic reticulum, inguinal, intravesical, topical (e.g., powder, ointment, or drops), or nasal routes. However, for many therapeutic uses, the preferred route/mode of administration is parenteral (e.g., intravenous, subcutaneous, intraperitoneal, intramuscular). The skilled artisan will understand that the route and/or manner of administration will vary depending on the intended purpose. In some preferred embodiments, the ADCs and pharmaceutical compositions described herein is administered by intravenous infusion or injection.

In certain embodiments, the pharmaceutical composition may also comprise additional pharmaceutically active agents. In certain embodiments, the additional pharmaceutically active agent is a drug having antineoplastic activity. In certain embodiments, the additional pharmaceutically active agent is selected from the group consisting of EGFR inhibitors, HER2 inhibitors, HER3 inhibitors, HER4 inhibitors, IGFR-1 inhibitors, mTOR inhibitors, PI3 kinase inhibitors, PTK7 inhibitors, c-met or VEGF inhibitors, chemotherapy drugs, or any combination thereof. In certain embodiments, the ADCs described herein and the additional pharmaceutically active agent are provided as separate components or as admixed components. Accordingly, the antibody or antigen-binding fragment thereof of the invention and the additional pharmaceutically active agent may be administered simultaneously, separately or sequentially.

### Methods of Use

The antibody-drug conjugate described herein, drug-linker described herein, or pharmaceutical compositions thereof can be used for treating various diseases or conditions, such as cancers with high expression of PTK7, including solid tumors or hematological malignancies, such as, lung cancer, breast cancer, epidermal cancer (for example, squamous cell carcinoma of skin, and oral squamous cell carcinoma), ovarian cancer, or esophageal cancer (for example, esophageal squamous cell carcinoma).

Therefore, the present application provides use of the antibody-drug conjugate (ADC), the drug-linker, or the pharmaceutical composition containing the same as described in any one of foregoing embodiments, in the manufacture of a medicament for the treatment of a cancer with high expression of PTK7.

At the same time, the present application also provides a method for treating a cancer with high expression of PTK7, which comprises a step of administering to a subject in need thereof a therapeutically effective amount of the antibody-drug conjugate (ADC), the drug-linker, or the pharmaceutical composition comprising the same as described in any one of foregoing embodiments.

In certain embodiments, the antibody-drug conjugate (ADC), drug-linker, or pharmaceutical composition is sufficient (e.g., in a subject) to:
(1) Inhibit the proliferation of cells (such as tumor cells);
(2) inhibit tumor growth;
(3) Induce and/or increase antibody-dependent cytotoxic activity;
(4) Inhibit PTK7-mediated signal transduction;
(5) Prevention and/or treatment of PTK7-mediated diseases/disorders; or
(6) Any combination of the above (1)-(5).

In certain embodiments, the PTK7-mediated disease/disorder is a tumor, e.g., a PTK7-expressing tumor. In certain embodiments, the tumor is selected from lung cancer, breast cancer, epidermal cancer (for example, squamous cell carcinoma of skin, and oral squamous cell carcinoma), ovarian cancer, or esophageal cancer (for example, esophageal squamous cell carcinoma), or any combination thereof.

### EXAMPLES

The following descriptions of specific embodiments will further illustrate the present invention, but this is not a limitation to the present invention. Those skilled in the art can make various modifications or improvements according to the teaching of the present invention without departing from the basic idea and scope of the present invention.

Information about the sequences involved in the present invention is described in the following table:

| Sequence | | |
|---|---|---|
| No. | Sequence name | Sequence information |
| 1 | 101A6 VH | EVKLVESGGGLVQPGGSLSLSCAASGFTFTDYYLS |
| | amino acid | WVRQPPGKALEWLALIRNKANGYTTEYSASVKG |
| | sequence | RFTISRDSSQSILYLQMDALRAEDSATYYCARDTL |
| | | AAYWGQGTSVTVSS |
| 2 | 101A6 VL | DIVMTQSHKFMSTSVGDRVSFTCKASQDVGSAVI |
| | amino acid | WYQQKPGQSPKLLIYWASTRHTGVPDRFTGSGSG |
| | sequence | TDFTLTISNVQSEDLADYFCQQYSSYPLTFGAGTK |
| | | LEIK |
| 3 | 101A6HZ/ | EVQLVESGGGLVQPGGSLRLSCAASGFTFTDYYL |
| | 101A6HZm VH | SWVRQAPGKGLEWLALIRNKANGYTTEYAASVK |
| | amino acid | GRFTISRDSSKNSLYLQMNSLKTEDTAVYYCARD |
| | sequence | TLAAYWGQGTLVTVSS |
| 4 | 101A6HZ/ | DIVMTQSPDSLAVSLGERATINCKASQDVGSAVI |
| | 101A6HZm VL | WYQQKPGQSPKLLIYWASTRHTGVPDRFSGSGSG |
| | amino acid | TDFTLTISSLQAEDVAVYYCQQYSSYPLTFGQGTK |
| | sequence | LEIK |
| 5 | 64A10 VH | QVQLKESGPVLVAPSQSLSITCTVSGFSLTSYGVY |
| | amino acid | WVRQPPGKGPEWLGVIWTSGNTNYNSALMSRLSI |
| | sequence | SKDNSKSQVFLKMNSLQTDDTAMYYCVKHDGG |
| | | NYVGVMNYWGQGTSVTVSS |
| 6 | 64A10 VL | DIQMTQSPATLSVTPGDRVSLSCRASQSISDYLHW |
| | amino acid | YQQKSQESPRLLIKYASQSISGIPSRFSGSGSGSDFT |
| | sequence | LSINSVEPEDVGVYYCQNGHSFPPTFGGGTKLEIK |
| 7 | 64A10HZ VH | QVQLQESGPGLVKPSETLSLTCTVSGFSLTSYGVY |
| | amino acid | WVRQPPGKGPEWLGVIWTSGNTNYNPSLKSRLTI |
| | sequence | SKDNSKNQVSLKLSSVTAADTAVYYCVKHDGGN |
| | | YVGVMNYWGQGTLVTVSS |
| 8 | 64A10HZ VL | EIVMTQSPDFQSVTPKEKVTITCRASQSISDYLHW |
| | amino acid | YQQKPDQSPKLLIKYASQSISGVPSRFSGSGSGSDF |
| | sequence | TLTINSLEAEDAATYYCQNGHSFPPTFGQGTKLEI |
| | | K |
| 9 | 64A10HZ05 VH | QVQLQESGPGLVKPSETLSLTCTVSGFSLTSYGVY |
| | amino acid | WVRQPPGKGLEWLGVIWTSGNTNYNPSLKSRLTI |
| | sequence | SKDNSKNQVSLKLSSVTAADTAVYYCVKHDGGN |
| | | YVGVMNYWGQGTLVTVSS |
| 10 | 64A10HZ05 VL | DIQMTQSPSSLSASVGDRVTITCRASQSISDYLHW |
| | amino acid | YQQKPGKAPKLLIKYASQSISGVPSRFSGSGSGSD |
| | sequence | FTLTISSLQPEDFATYYCQNGHSFPPTFGGGTKVEI |
| | | K |
| 11 | 101A6/101A6HZ/ | GFTFTDY |
| | 101A6HZm | |
| | Chothia CDR | |
| | H1 | |
| 12 | 101A6/101A6HZ/ | RNKANGYT |
| | 101A6HZm | |
| | Chothia CDR | |
| | H2 | |
| 13 | 101A6/101A6HZ/ | DTLAAY |
| | 101A6HZm | |
| | Chothia/kabat/ | |
| | AbM CDR H3 | |
| 14 | 101A6/101A6HZ/ | KASQDVGSAVI |
| | 101A6HZm | |
| | Chothia/Kabat/ | |
| | AbM CDR L1 | |
| 15 | 101A6/101A6HZ/ | WASTRHT |
| | 101A6HZm | |
| | Chothia/Kabat/ | |
| | AbM CDR L2 | |
| 16 | 101A6/101A6HZ/ | QQYSSYPLT |
| | 101A6HZm | |
| | Chothia/Kabat/ | |
| | IMGT/AbM | |
| | CDR L3 | |
| 17 | 101A6/101A6HZ/ | DYYLS |
| | 101A6HZm | |
| | Kabat CDR H1 | |
| 18 | 101A6HZ/ | LIRNKANGYTTEYAASVKG |
| | 101A6HZm Kabat | |
| | CDR H2 | |
| 19 | 101A6 Kabat | LIRNKANGYTTEYSASVKG |
| | CDR H2 | |
| 20 | 101A6/101A6HZ/ | GFTFTDYY |
| | 101A6HZm | |
| | IMGT CDR H1 | |
| 21 | 101A6/101A6HZ/ | IRNKANGYTT |
| | 101A6HZm | |
| | IMGT CDR H2 | |
| 22 | 101A6/101A6HZ/ | ARDTLAAY |
| | 101A6HZm | |
| | IMGT CDR H3 | |
| 23 | 101A6/101A6HZ/ | QDVGSA |
| | 101A6HZm | |
| | IMGT CDR L1 | |
| 24 | 101A6/101A6HZ/ | WAS |
| | 101A6HZm | |
| | IMGT CDR L2 | |
| 25 | 101A6/101A6HZ/ | GFTFTDYYLS |
| | 101A6HZm | |
| | AbM CDR H1 | |
| 26 | 101A6/101A6HZ/ | LIRNKANGYTTE |
| | 101A6HZm | |
| | AbM CDR H2 | |
| 27 | 64A10/64A10HZ/ | GFSLTSY |
| | 64A10HZ05 | |
| | Chothia CDR | |
| | H1 | |
| 28 | 64A10/64A10HZ/ | WTSGN |
| | 64A10HZ05 | |
| | Chothia CDR | |
| | H2 | |
| 29 | 64A10/64A10HZ/ | HDGGNYVGVMNY |
| | 64A10HZ05 | |
| | Chothia/Kabat/ | |
| | AbM CDR H3 | |
| 30 | 64A10/64A10HZ/ | RASQSISDYLH |
| | 64A10HZ05 | |
| | Chothia/Kabat/ | |
| | AbM CDR L1 | |
| 31 | 64A10/64A10HZ/ | YASQSIS |
| | 64A10HZ05 | |
| | Chothia/Kabat/ | |
| | AbM CDR L2 | |
| 32 | 64A10/64A10HZ/ | QNGHSFPPT |
| | 64A10HZ05 | |
| | Chothia/Kabat/ | |
| | IMGT/AbM | |
| | CDR L3 | |
| 33 | 64A10/64A10HZ/ | SYGVY |
| | 64A10HZ05 | |
| | Kabat CDR H1 | |
| 34 | 64A10HZ/ | VIWTSGNTNYNPSLKS |
| | 64A10HZ05 Kabat | |
| | CDR H2 | |
| 35 | 64A10 Kabat | VIWTSGNTNYNSALMS |
| | CDR H2 | |
| 36 | 64A10/64A10HZ/ | GFSLTSYG |
| | 64A10HZ05 | |
| | IMGT CDR H1 | |
| 37 | 64A10/64A10HZ/ | IWTSGNT |
| | 64A10HZ05 | |
| | IMGT CDR H2 | |
| 38 | 64A10/64A10HZ/ | VKHDGGNYVGVMNY |
| | 64A10HZ05 | |
| | IMGT CDR H3 | |
| 39 | 64A10/64A10HZ/ | QSISDY |
| | 64A10HZ05 | |
| | IMGT CDR L1 | |
| 40 | 64A10/64A10HZ/ | YAS |
| | 64A10HZ05 | |
| | IMGT CDR L2 | |
| 41 | 64A10/64A10HZ/ | GFSLTSYGVY |
| | 64A10HZ05 | |
| | AbM CDR H1 | |
| 42 | 64A10/64A10HZ/ | VIWTSGNTN |
| | 64A10HZ05 | |
| | AbM CDR H2 | |
| 43 | Human heavy | ASTKGPSVFPLAPSSKSTSGGTAALGCLVKDYFPE |
| | chain constant | PVTVSWNSGALTSGVHTFPAVLQSSGLYSLSSVV |
| | region IgG1 | TVPSSSLGTQTYICNVNHKPSNTKVDKKVEPKSC |
| | | DKTHTCPPCPAPELLGGPSVFLFPPKPKDTLMISRT |
| | | PEVTCVVVDVSHEDPEVKFNWYVDGVEVHNAKT |
| | | KPREEQYNSTYRVVSVLTVLHQDWLNGKEYKCK |
| | | VSNKALPAPIEKTISKAKGQPREPQVYTLPPSRDEL |
| | | TKNQVSLTCLVKGFYPSDIAVEWESNGQPENNYK |
| | | TTPPVLDSDGSFFLYSKLTVDKSRWQQGNVFSCS |
| | | VMHEALHNHYTQKSLSLSPGK |
| 44 | Human light | RTVAAPSVFIFPPSDEQLKSGTASVVCLLNNFYPR |
| | chain constant | EAKVQWKVDNALQSGNSQESVTEQDSKDSTYSL |
| | region CL | SSTLTLSKADYEKHKVYACEVTHQGLSSPVTKSF |
| | | NRGEC |
| 45 | Mutant human | ASTKGPSVFPLAPSSKSTSGGTAALGCLVKDYFPE |
| | heavy chain | PVTVSWNSGALTSGVHTFPAVLQSSGLYSLSSVV |
| | constant region | TVPSSSLGTQTYICNVNHKPSNTKVDKKVEPKSC |
| | IgG1 | DKTHTCPPCPAPEAAGAPSVFLFPPKPKDTLMISR |
| | | TPEVTCVVVDVSHEDPEVKFNWYVDGVEVHNAK |
| | | TKPREEQYNSTYRVVSVLTVLHQDWLNGKEYKC |
| | | KVSNKALPAPIEKTISKAKGQPREPQVYTLPPSRD |
| | | ELTKNQVSLTCLVKGFYPSDIAVEWESNGQPENN |
| | | YKTTPPVLDSDGSFFLYSKLTVDKSRWQQGNVFS |
| | | CSVMHEALHNHYTQKSLSLSPGK |
| 46 | 101A6HZm HC | EVQLVESGGGLVQPGGSLRLSCAASGFTFTDYYL |
| | | SWVRQAPGKGLEWLALIRNKANGYTTEYAASVK |
| | | GRFTISRDSSKNSLYLQMNSLKTEDTAVYYCARD |
| | | TLAAYWGQGTLVTVSSASTKGPSVFPLAPSSKSTS |
| | | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPEAAGAPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| 47 | 101A6HZ/ | DIVMTQSPDSLAVSLGERATINCKASQDVGSAVI |
| | 101A6HZm LC | WYQQKPGQSPKLLIYWASTRHTGVPDRFSGSGSG |
| | | TDFTLTISSLQAEDVAVYYCQQYSSYPLTFGQGTK |
| | | LEIKRTVAAPSVFIFPPSDEQLKSGTASVVCLLNNF |
| | | YPREAKVQWKVDNALQSGNSQESVTEQDSKDST |
| | | YSLSSTLTLSKADYEKHKVYACEVTHQGLSSPVT |
| | | KSFNRGEC |
| 48 | Linker | GGFG |
| 49 | Linker | GFLG |
| 50 | Linker | GGVA |
| 51 | Linker | GGGGG |
| 52 | 101A6HZm HC | XVQLVESGGGLVQPGGSLRLSCAASGFTFTDYYL |
| | heavy chain | SWVRQAPGKGLEWLALIRNKANGYTTEYAASVK |
| | amino acid | GRFTISRDSSKNSLYLQMNSLKTEDTAVYYCARD |
| | sequence | TLAAYWGQGTLVTVSSASTKGPSVFPLAPSSKSTS |
| | (Pyroglutamate | GGTAALGCLVKDYFPEPVTVSWNSGALTSGVHTF |
| | or Pyroglutamic | PAVLQSSGLYSLSSVVTVPSSSLGTQTYICNVNHK |
| | acid at N- | PSNTKVDKKVEPKSCDKTHTCPPCPAPEAAGAPS |
| | terminus; no C- | VFLFPPKPKDTLMISRTPEVTCVVVDVSHEDPEVK |
| | terminal lysine) | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PG |
| | | X is pyroglutamate or pyroglutamic acid |
| 53 | 101A6HZm HC | EVQLVESGGGLVQPGGSLRLSCAASGFTFTDYYL |
| | heavy chain | SWVRQAPGKGLEWLALIRNKANGYTTEYAASVK |
| | amino acid | GRFTISRDSSKNSLYLQMNSLKTEDTAVYYCARD |
| | sequence (no C- | TLAAYWGQGTLVTVSSASTKGPSVFPLAPSSKSTS |
| | terminal lysine) | GGTAALGCLVKDYFPEPVTVSWNSGALTSGVHTF |
| | | PAVLQSSGLYSLSSVVTVPSSSLGTQTYICNVNHK |
| | | PSNTKVDKKVEPKSCDKTHTCPPCPAPEAAGAPS |
| | | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PG |
| 54 | 101A6HZm HC | XVQLVESGGGLVQPGGSLRLSCAASGFTFTDYYL |
| | heavy chain | SWVRQAPGKGLEWLALIRNKANGYTTEYAASVK |
| | amino acid | GRFTISRDSSKNSLYLQMNSLKTEDTAVYYCARD |
| | sequence | TLAAYWGQGTLVTVSSASTKGPSVFPLAPSSKSTS |
| | (Pyroglutamate | GGTAALGCLVKDYFPEPVTVSWNSGALTSGVHTF |
| | or Pyroglutamic | PAVLQSSGLYSLSSVVTVPSSSLGTQTYICNVNHK |
| | acid at N- | PSNTKVDKKVEPKSCDKTHTCPPCPAPEAAGAPS |
| | terminus) | VFLFPPKPKDTLMISRTPEVTCVVVDVSHEDPEVK |
| | | FNWYVDGVEVHNAKTKPREEQYNSTYRVVSVLT |
| | | VLHQDWLNGKEYKCKVSNKALPAPIEKTISKAKG |
| | | QPREPQVYTLPPSRDELTKNQVSLTCLVKGFYPSD |
| | | IAVEWESNGQPENNYKTTPPVLDSDGSFFLYSKLT |
| | | VDKSRWQQGNVFSCSVMHEALHNHYTQKSLSLS |
| | | PGK |
| | | X is pyroglutamate or pyroglutamic acid |
| 55 | Linker | GGGGS |
| 56 | Linker | GGGGSGGGGSGGGGSGGGGS |

Abbreviations herein have the following meanings:

| Abbreviation | Meaning | Abbreviation | Meaning |
|---|---|---|---|
| HATU | N,N,N′,N′-tetramethyl-O-(7-azabenzotriazol- | NBS | N-Bromosuccinimide |
| | 1-yl)urea hexafluorophosphate | | |
| DIPEA | Diisopropylethylamine | THF | Tetrahydrofuran |
| HPLC | High-performance liquid chromatography | DMSO | Dimethyl sulfoxide |
| TFA | Trifluoroacetate | DMF | N,N-dimethylformamide |
| Pd/C | Palladium on carbon | PyroE | Pyroglutamate |

The structures of the compounds described in the following Examples were determined by nuclear magnetic resonance (¹H NMR) or mass spectrometry (MS).

The determination of nuclear magnetic resonance (¹H NMR) was carried out through a Bruker 400 MHz nuclear magnetic resonance instrument; a deuterated reagent was hexadeuteriodimethylsulfoxide (DMSO-d6); and an internal standard was tetramethylsilane (TMS).

Abbreviations in nuclear magnetic resonance (NMR) spectra used in Examples are shown below.
s: singlet, d: doublet, t: triplet, q: quartet, m: multiplet, br: broad, J: coupling constant, Hz: Hertz, and DMSO-d6: deuterated dimethylsulfoxide. 8 value was expressed in ppm.

The determination of mass spectrum (MS) was carried out using Agilent (ESI) mass spectrometer, with model of Agilent 6120B.

### Example 1 N—((S)-10-benzyl-1-((1S,9S)-9-ethyl-5-fluoro-9-hydroxyl-4-methyl-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentaoxo-3-oxa-5,8,11,14-tetraazahexadecan-16-yl-6-(2,5-dioxo-2,5-dihydro-1-H-pyrrol-1-yl)hexanamide (A-01)

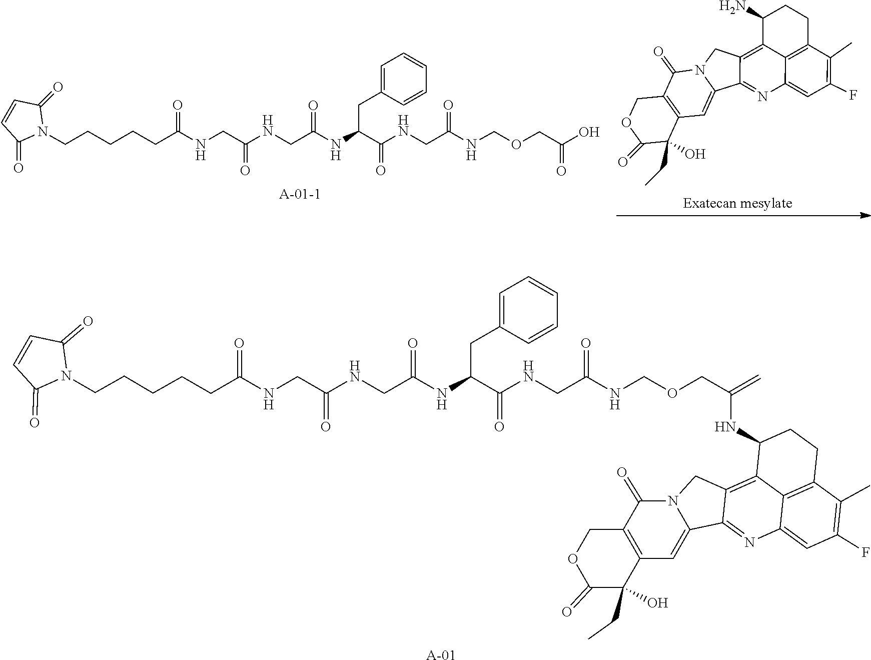 [SMILES: O=C(O)COCNC(=O)CNC(=O)[C@H](Cc1ccccc1)NC(=O)CNC(=O)CNC(=O)CCCCCN1C(=O)C=CC1=O ; CC[C@@]1(O)C(=O)OCc2c1cc1n(c2=O)Cc2c-1nc1cc(F)c(C)c3c1c2[C@@H](N)CC3 ; C=C(COCNC(=O)CNC(=O)[C@H](Cc1ccccc1)NC(=O)CNC(=O)CNC(=O)CCCCCN1C(=O)C=CC1=O)N[C@H]1CCc2c(C)c(F)cc3nc4c(c1c23)Cn1c-4cc2c(c1=O)COC(=O)[C@]2(O)CC ; "A-01"]

A compound A-01-1 (0.40 g, 640.59 μmol, its synthesis is referred to Patent CN 111936169A) and exatecan mesylate (0.37 g, 704.65 μmol) were dissolved in DMF (8 mL); and HATU (0.32 g, 832.77 μmol) and DIPEA (0.25 g, 1.92 mmol) were added to react at 25 °C for 4 h. DIPEA was removed under reduced pressure, freeze-drying was carried out with water to remove most of DMF to obtain a crude product, and the crude product was purified by preparative high-performance liquid chromatography (conditions as follows) to obtain 273 mg of the title compound.

Chromatographic column: Waters XBridge Prep
C18 OBD 45 mm × 450 mm × 8.0 μm
Mobile phase A: acetonitrile; mobile
phase B: water (0.05% trifluoroacetic acid)

| Time | Mobile phase A | Mobile phase B | Flow rate |
|---|---|---|---|
| [min] | [%] | [%] | [mL/min] |
| 0.00 | 30 | 70 | 70 |
| 5.00 | 30 | 70 | 70 |
| 60.00 | 70 | 30 | 70 |

The structural characterization data are as follows:

ESI-MS (m/z): 1034.4 [M+H]⁺.

### Example 2 Synthesis of N-((1S,9S)-5-chloro-9-ethyl-9-hydroxyl-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide and N-((1R,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide (1-8-A and 1-8-B)

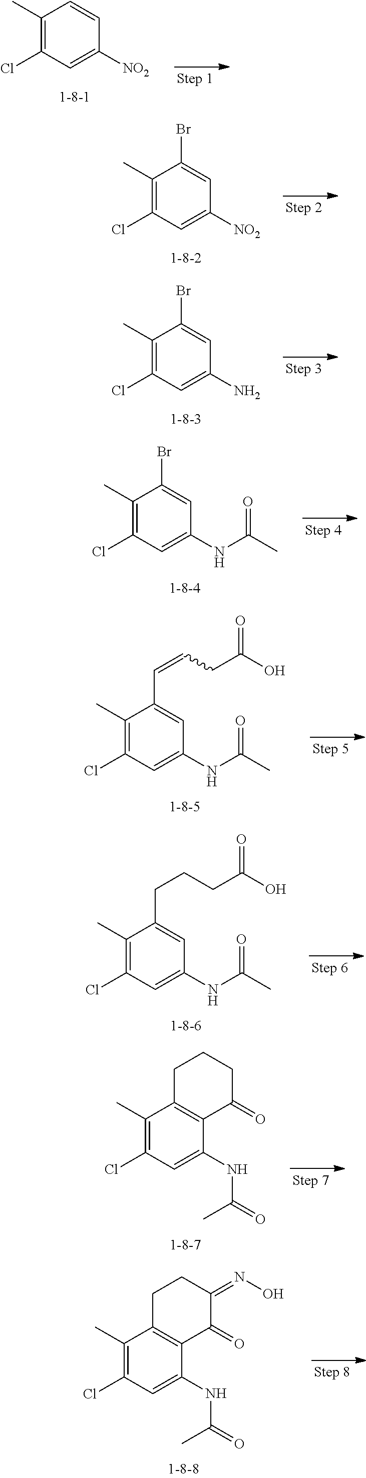

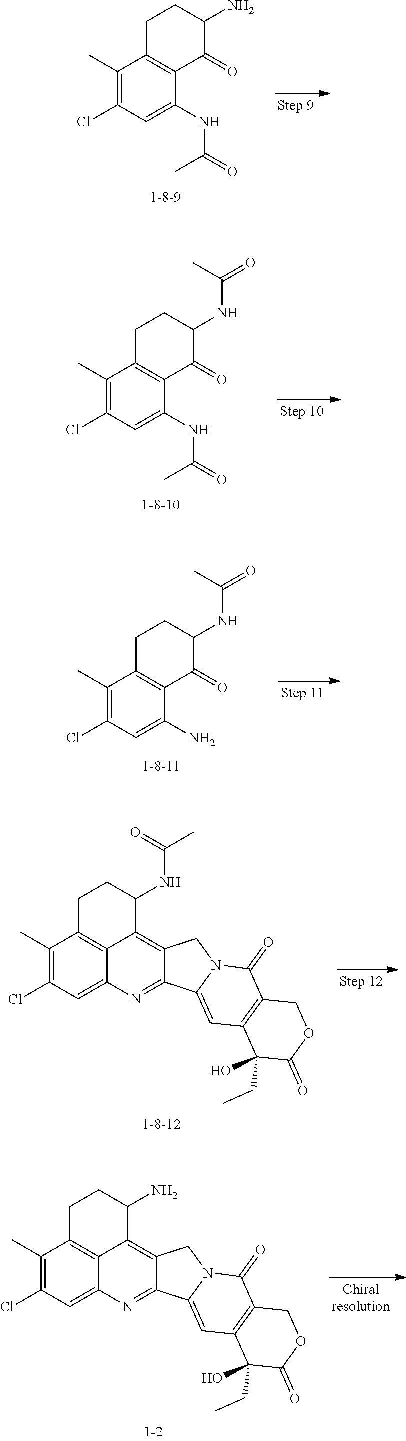 [SMILES: CC(=O)Nc1cc(Cl)c(C)c2c1C(=O)C(N)CC2 ; CC(=O)Nc1cc(Cl)c(C)c2c1C(=O)C(NC(C)=O)CC2 ; CC(=O)NC1CCc2c(C)c(Cl)cc(N)c2C1=O ; CC[C@@]1(O)C(=O)OCc2c1cc1n(c2=O)Cc2c-1nc1cc(Cl)c(C)c3c1c2C(NC(C)=O)CC3 ; CC[C@@]1(O)C(=O)OCc2c1cc1n(c2=O)Cc2c-1nc1cc(Cl)c(C)c3c1c2C(N)CC3]

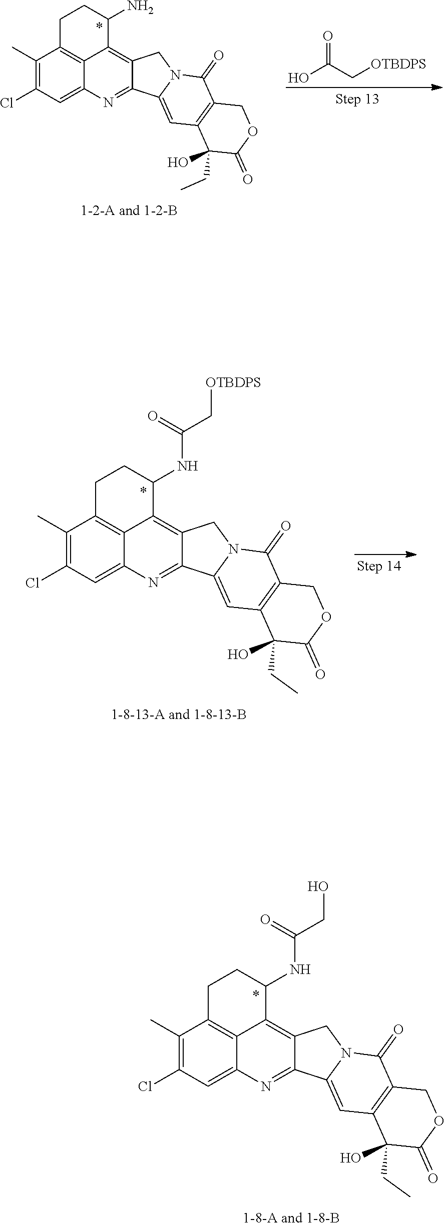

* represents that the atoms at this position are R or S in chirality

### Step 1: Synthesis of 1-chloro-3-bromo-2-methyl-5-nitrobenzene (1-8-2)

The compound 1-8-1 (5.00 g, 29.14 mmol) was dissolved in n-heptane (25 mL) at temperature below 25 °C; concentrated sulfuric acid (25 mL) was added and heated to 50 °C; NBS was added in batches at 50 °C (6.22 g, 34.97 mmol), and the temperature was kept at 50 °C for reaction for 2 h; and the reaction was monitored by thin-layer chromatography (ethyl acetate: petroleum ether=1:10). The reaction solution was cooled to room temperature, then added dropwise to ice water, and extracted with toluene; organic phases were combined, washed with sodium sulfite solution, water, and saturated brine respectively, dried over anhydrous sodium sulfate, and concentrated under reduced pressure; and the crude product was purified by preparative high-performance liquid chromatography (the conditions shown as follows), and the resulting solution was freeze-dried to obtain 4.88 g of the title compound.

Chromatographic column: C18 ODS 45
mm × 450 mm × 8.0 μm
Mobile phase A: acetonitrile; mobile
phase B: water (0.05% formic acid)

| Time | Mobile phase A | Mobile phase B | Flow rate |
|---|---|---|---|
| [min] | [%] | [%] | [mL/min] |
| 0 | 60 | 40 | 60 |
| 10 | 60 | 40 | 60 |
| 40 | 100 | 0 | 60 |

### Step 2: Synthesis of 3-chloro-5-bromo-4-methylaniline (1-8-3)

The compound 1-8-2 (4.88 g, 19.48 mmol) was dissolved in ethyl acetate (100 mL) at 25 °C, and platinum on carbon (2.00 g, 19.48 mmol, 5% of content) was added. The reaction was carried out at 60 °C for 4 h under hydrogen protection after hydrogen replacement, and the reaction was monitored by high-performance liquid chromatography-mass spectrometry. The reaction solution was filtered and concentrated to obtain 3.68 g of the crude product of the title compound, which was directly used in the next reaction without further purification.

### Step 3: Synthesis of N-(3-chloro-5-bromo-4-methylphenyl)acetamide (1-8-4)

The compound 1-8-3 (3.63 g, 14.82 mmol) was dissolved in ethyl acetate (70 mL) at 20 °C; triethylamine (4.50 g, 44.45 mmol) and acetic anhydride (2.27 g, 22.23 mmol) were added, the reaction was kept at 20 °C for 20 h, and was monitored by high-performance liquid chromatography-mass spectrometry. Water was added to the reaction liquid; extraction was carried out with ethyl acetate; the organic phases were combined, dried over anhydrous sodium sulfate, and concentrated under reduced pressure to obtain a crude product, which was slurried in a mixture of ethyl acetate:petroleum ether=1:5 to obtain 2.86 g of the title compound.

### Step 4: Synthesis of 4-(5-acetamido-3-chloro-2-methylphenyl)but-3-enoic acid (1-8-5)

The compound 1-8-4 (1.80 g, 6.86 mmol) was dissolved in THF (20 mL) and water (5 mL) at 20 °C; vinylacetic acid (708.31 mg, 8.23 mmol), DIPEA (1.95 g, 15.08 mmol) and tris(o-methylphenyl) phosphorus (62.60 mg, 0.20 mmol) were added; and the reaction system was flushed with nitrogen and then heated to 70 °C for 5 h; and the reaction was monitored by HPLC-MS. 1 N of sodium hydroxide solution was added to the reaction solution to adjust pH=8; ethyl acetate was added for extraction; the aqueous phase was adjusted with 1 N of hydrochloric acid until reaching pH=3, and then extracted with ethyl acetate; and the organic phases were combined, dried over anhydrous sodium sulfate, and concentrated under reduced pressure to obtain 0.82 g of the title compound, which was directly used in the next reaction.

### Step 5: Synthesis of 4-(5-acetamido-3-chloro-2-methylphenyl)butyric acid (1-8-6)

The compound 1-8-5 (2.60 g, 9.71 mmol) was dissolved in THF (50 mL) at 20 °C; Pd/C (0.52 g, content 10%) was added; the system was placed under positive hydrogen pressure using a balloon filled with hydrogen gas, and reacted at 40 °C for 2 h; and the reaction was monitored by high performance liquid chromatography-mass spectrometry. The reaction solution was filtered, and the filter was concentrated to obtain 2.43 g of the title compound, which was directly used in the next reaction without further purification.

### Step 6: Synthesis of N-(3-chloro-4-methyl-8-oxo-5,6,7,8-tetrahydronaphthalen-1-yl)acetamide (1-8-7)

The compound 1-8-6 (2.43 g, 9.01 mmol) was dissolved in trifluoroacetic acid (10 mL), and cooled to 5 °C; trifluoroacetic anhydride (3.78 g, 18.02 mmol, 2.50 mL) was added dropwise; the temperature was maintained at 5 °C for reaction for 4 h; and the reaction was monitored by high-performance liquid chromatography-mass spectrometry. The reaction solution was added to water, and dissolved in 10 N sodium hydroxide to adjust pH=9; ethyl acetate was added for extraction; the organic phases were combined, dried over anhydrous sodium sulfate, concentrated under reduced pressure, and purified by silica gel column (ethyl acetate:petroleum ether=0-20%) to obtain 1.53 g of the title compound.

### Step 7: Synthesis of (Z)—N-(3-chloro-7-(hydroxyimino)-4-methyl-8-oxo-5,6,7,8-tetrahydronaphthalen-1-yl)acetamide (1-8-8)

Potassium tert-butoxide (1.50 g, 13.37 mmol) was dissolved in THF (16 mL) and tert-butanol (4 mL) at 5 °C; a compound 1-8-7 (1.53 g, 6.08 mmol) in the THF solution (16 mL) was added dropwise; after 10 min, amyl nitrite (1.14 g, 9.73 mmol) was added dropwise; and the reaction was kept at 5 °C for 1 h and was monitored by high-performance liquid chromatography-mass spectrometry. The reaction solution was adjusted to pH=5 with 1 N of hydrochloric acid, and extracted with ethyl acetate; the organic phases were combined, dried over anhydrous sodium sulfate, and concentrated under reduced pressure; and the crude product was slurried with methyl tert-butyl ether to obtain 1.20 g of the title compound.

### Step 8: Synthesis of N-(7-amino-3-chloro-4-methyl-8-oxo-5,6,7,8-tetrahydronaphthalen-1-yl)acetamide (1-8-9)

A compound 1-8-8 (0.50 g, 1.78 mmol) was dissolved in methyl alcohol (8 mL) and 2 N of hydrochloric acid (8 mL) at 20 °C; Pd/C (0.15 g, content 10%) was added; the system was placed under positive hydrogen pressure using a balloon filled with hydrogen gas, reacted at 5 °C for 2 h; and the reaction was monitored by high-performance liquid chromatography-mass spectrometry. The reaction solution was filtered and concentrated to obtain 0.52 g of the title compound, which was directly used in the next reaction without further purification.

### Step 9: Synthesis of N,N′-(3-chloro-4-methyl-8-oxo-5,6,7,8-tetrahydronaphthalene-1,7-diyl)diacetamide (1-8-10)

The compound 1-8-9 (0.52 g, 1.70 mmol) was dissolved in pyridine (5 mL) at 20 °C; acetic anhydride was added (2 mL); and the reaction was maintained at 20 °C for 2 h, and was monitored by high-performance liquid chromatography-mass spectrometry. The reaction solution was added to water; ethyl acetate was added for extraction; the organic phases were washed with water, combined, dried over anhydrous sodium sulfate, concentrated under reduced pressure, and purified by silica gel column (ethyl acetate:petroleum ether=0-30%) to obtain 0.22 g of the title compound.

### Step 10: Synthesis of N-(8-amino-6-chloro-5-methyl-1-oxo-1,2,3,4-tetrahydronaphthalene-2-yl)acetamide (1-8-11)

The compound 1-8-10 (450.97 mg, 1.46 mmol) was dissolved in methanol (16 mL) at 20 °C; 2 N of hydrochloric acid (16 mL) was added, and heated to 60 °C for 2 h; and the reaction was monitored by high-performance liquid chromatography-mass spectrometry. Saturated sodium bicarbonate solution was added to the cooled reaction solution to adjust the pH=8; ethyl acetate was added for extraction; the organic phases were combined, dried over anhydrous sodium sulfate, and concentrated under reduced pressure to obtain 230.00 mg of the title compound, which was used directly in the next step without further purification.

### Step 11: Synthesis of N-((9S)-5-chloro-9-ethyl-9-hydroxy-4-methyl-10,13-dioxo-2,3,9,10,13,15-hexahydro-1H,12H-benzopyrano[3′,4′:6,7]indolizino[1,2-b]quinolin-1-yl)acetamide (1-8-12)

The compound 1-8-11 (230.00 mg, 0.78 mmol) was dissolved in toluene (10 mL); (S)-4-ethyl-4-hydroxy-7,8-dihydro-1H-pyrano[3,4-f]indolizine-3,6,10 (4H)-trione (230.00 mg, 0.87 mmol) and p-toluenesulfonic acid (26.73 mg, 0.16 mmol) were added, and heated to 140 °C for reaction for 5 h; and the reaction was monitored by high-performance liquid chromatography-mass spectrometry. The reaction solution was concentrated, and the crude product was purified by silica gel column (methanol:dichloromethane=0-10%) to obtain 150.00 mg of the title compound.

### Step 12: Synthesis of (9S)-1-amino-5-chloro-9-ethyl-9-hydroxy-4-methyl-1,2,3,9,12,15-hexahydro-10H,13H-benzopyrano[3′,4′:6,7]indolizino[1,2-b]quinolin-10,13-dione (1-2)

The compound 1-8-12 (40.00 mg, 0.081 mmol) was added into concentrated hydrochloric acid (1 mL), and heated to 100 °C for 5 h; and the reaction was monitored by high-performance liquid chromatography-mass spectrometry. The reaction solution was filtered, the filtrate was purified by preparative high-performance liquid chromatography (conditions shown as follows), and the resulting solution was freeze-dried to obtain the title compound 1-2 hydrochloride. Compound 1-2 hydrochloride was separated under the following purification conditions to obtain two isomers, named 1-2-A (5.00 mg of trifluoroacetate, retained for 9.85 min) and 1-2-B (7.00 mg of trifluoroacetate, retained for 10.62 min) according to the retention time.

Chromatographic column: SunFire Prep C18
OBD 19 mm × 150 mm × 5.0 μm
Mobile phase A: acetonitrile; mobile phase B:
water (0.05% trifluoroacetic acid) phase A

| Time | Mobile phase A | Mobile phase B | Flow rate |
|---|---|---|---|
| [min] | [%] | [%] | [mL/min] |
| 0 | 5 | 95 | 28 |
| 2 | 5 | 95 | 28 |
| 18 | 50 | 50 | 28 |

The structural characterization data are as follows:

1-2-A:

¹H NMR (400 MHz, DMSO-d6) δ 8.42 (s, 3H), 8.27 (s, 1H), 7.36 (s, 1H), 6.59 (s, 1H), 5.78-5.63 (m, 1H), 5.50-5.36 (m, 3H), 5.10-5.06 (m, 1H), 3.20-3.04 (m, 2H), 2.56 (s, 3H), 2.26-2.13 (m, 2H), 1.93-1.79 (m, 2H), 0.88 (t, J=7.2 Hz, 3H).

ESI-MS (m/z): 452.1 [M+H]⁺.

1-2-B:

¹H NMR (400 MHz, DMSO-d6) δ 8.42 (s, 3H), 8.27 (s, 1H), 7.36 (s, 1H), 6.58 (s, 1H), 5.78-5.63 (m, 1H), 5.50-5.36 (m, 3H), 5.10-5.06 (m, 1H), 3.20-3.04 (m, 2H), 2.55 (s, 3H), 2.26-2.13 (m, 2H), 1.93-1.79 (m, 2H), 0.88 (t, J=7.2 Hz, 3H).

ESI-MS (m/z): 452.0 [M+H]⁺.

### Step 13: Synthesis of 2-((tert-butyldiphenylsilyl)oxy)-N-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolazino[1,2-b]quinolin-1-yl)acetamide and 2-((tert-butyldiphenylsilyl)oxy)-N-((1R,9S)-5-chloro-9-ethyl-9-hydroxy-4-methyl-10,13-dioxo-2,3,9,10,13,15-hexahydro-1H,12H-benzo[de]pyrano[3′,4′:6,7]indolazino[1,2-b]quinolin-1-yl)acetamide (1-8-13-A and 1-8-13-B)

Hydrochloride salt of the compound 1-2 (40.00 mg, 81.91 μmol) was dissolved in N,N-dimethylformamide (1 mL) at 25 °C; 2-((tert-butyldiphenylsilyl)oxy) acetic acid (30.91 mg, 98.29 μmol), HATU (62.25 mg, 163.81 μmol) and N,N-diisopropylethylamine (42.34 mg, 327.63 μmol) were added; and the reaction was kept at 25 °C for 0.5 h, and was monitored by high-performance liquid chromatography-mass spectrometry. After the reaction was completed, water was added to the reaction solution; dichloromethane/methanol (v/v=10/1) was added for extraction; the organic phases were combined, dried over anhydrous sodium sulfate and concentrated under reduced pressure; and the crude product was purified by preparative thin-layer chromatography (dichloromethane:methanol=20:1) and separated to obtain two isomers; and according to the Rf value, the two isomers were named as 1-8-13-A (15.00 mg, Rf value of 0.3) and 1-8-13-B (12.00 mg, Rf value of 0.35).

### Step 14: Synthesis of N-((1S,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide and N-((1R,9S)-5-chloro-9-ethyl-9-hydroxy-4-methyl-10,13-dioxo-2,3,9,10,13,15-hexahydro-1H,12H-benzo[de]pyrano[3′,4′:6,7]indolizino[1,2-b]quinolin-1-yl)-2-hydroxyacetamide (1-8-A and 1-8-B)

1-8-13-A (15.00 mg) and 1-8-13-B (12.00 mg) were dissolved in tetrahydrofuran (1 mL) in two reaction flasks at 25 °C, respectively; tetrabutylammonium fluoride (1M in tetrahydrofuran)/glacial acetic acid mixture (v/v=13/1) (50 μL) was added dropwise; and the reaction was kept at 25 °C for 0.5 h, and was monitored by high-performance liquid chromatography-mass spectrometry. After the reaction was completed, the reaction solutions were purified by preparative high performance liquid chromatography respectively, and the resulting solutions were freeze-dried respectively to obtain the respective title compounds 1-8-A (i.e., 1-10) (6.94 mg) and 1-8-B (i.e., 1-9) (4.00 mg).

Chromatographic column: SunFire Prep C18
OBD 19 mm × 150 mm × 5.0 μm
Mobile phase A: acetonitrile; mobile
phase B: water (0.05% formic acid)
Time [min] Mobile phase A [%] Mobile
phase B [%] Flow rate [mL/min]

| Time | Mobile phase A | Mobile phase B | Flow rate |
|---|---|---|---|
| [min] | [%] | [%] | [mL/min] |
| 0 | 20 | 80 | 28 |
| 3 | 20 | 80 | 28 |
| 18 | 90 | 10 | 28 |

The structural characterization data of 1-8-A (i.e., 1-10) are as follows:

1H NMR (400 MHz, DMSO-d6) δ 8.43 (d, J=8.8 Hz, 1H), 8.16 (s, 1H), 7.31 (s, 1H), 6.55 (s, 1H), 5.65-5.36 (m, 4H), 5.21 (q, J=19.0 Hz, 2H), 3.95 (d, J=5.7 Hz, 2H), 3.26-3.11 (m, 2H), 2.53 (s, 3H), 2.30-2.08 (m, 2H), 1.94-1.79 (m, 2H), 0.87 (t, J=7.3 Hz, 3H).

ESI-MS (m/z): 510.1 [M+H]⁺.

The structural characterization data of 1-8-B (i.e., 1-9) are as follows:

1H NMR (400 MHz, DMSO-d6) δ 8.45 (d, J=8.9 Hz, 1H), 8.15 (s, 1H), 7.31 (s, 1H), 6.54 (s, 1H), 5.64-5.35 (m, 4H), 5.19 (q, J=19.0 Hz, 2H), 3.97 (d, J=5.2 Hz, 2H), 3.27-3.10 (m, 2H), 2.51 (s, 3H), 2.27-2.10 (m, 2H), 1.93-1.80 (m, 2H), 0.88 (t, J=7.3 Hz, 3H).

ESI-MS (m/z): 510.1 [M+H]⁺.

### Example 3 N-((10S)-10-benzyl-1-(((1S,9S)-5-chloro-9-ethyl-9-hydroxyl-4-methyl-10,13-dioxo-2,3,9,10,13,15-hexahydro-1H,12H-benzo[de]pyrano[3′,4′;6,7]indolizino[1,2-b]quinolin-1-yl)amino)-1,6,9,12,15-pentaoxo-3-oxa-5,8,11,14-tetraazahexadecan-16-yl)-6-(2-(methanesulfonyl)pyrimidin-5-yl)-hex-5-ynamide and N-((10S)-10-benzyl-1-(((1R,9S)-5-chloro-9-ethyl-9-hydroxy-4-methyl-10,13-dioxo-2,3,9,10,13,15-hexahydro-1H,12H-benzo[de]pyrano[3′,4′;6,7]indolizino[1,2-b]quinolin-1-yl)amino)-1,6,9,12,15-pentoxo-3-oxa-5,8,11,14-tetraazahexadecan-16-yl)-6-(2-(methanesulfonyl)pyrimidin-5-yl)-hex-5-ynamide (A-07-A (A-05) and A-07-B (A-06))

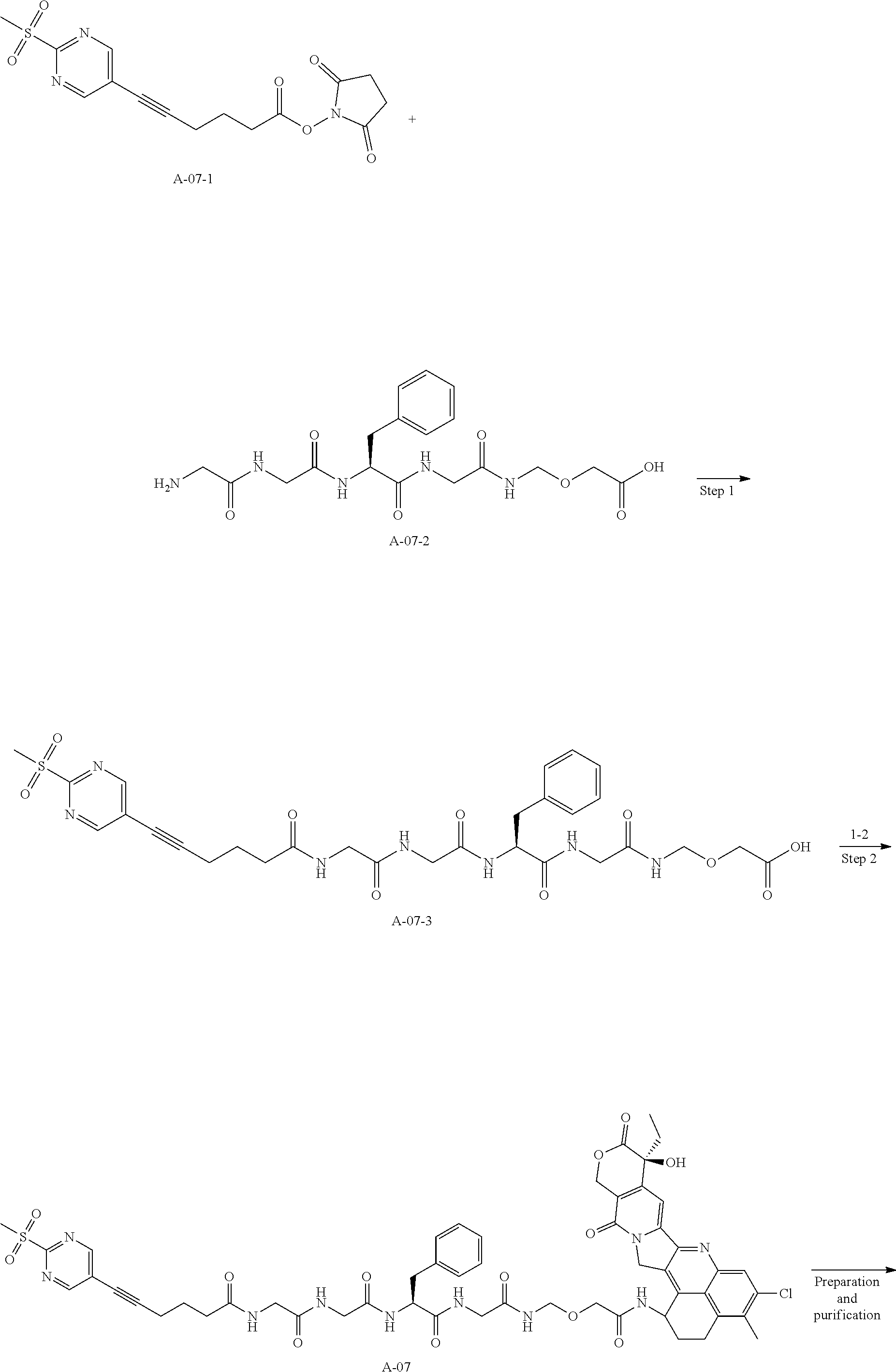 [SMILES: CS(=O)(=O)c1ncc(C#CCCCC(=O)ON2C(=O)CCC2=O)cn1 ; NCC(=O)NCC(=O)N[C@@H](Cc1ccccc1)C(=O)NCC(=O)NCOCC(=O)O ; CS(=O)(=O)c1ncc(C#CCCCC(=O)NCC(=O)NCC(=O)N[C@@H](Cc2ccccc2)C(=O)NCC(=O)NCOCC(=O)O)cn1 ; CC[C@@]1(O)C(=O)OCc2c1cc1n(c2=O)Cc2c-1nc1cc(Cl)c(C)c3c1c2C(NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; "A-07"]

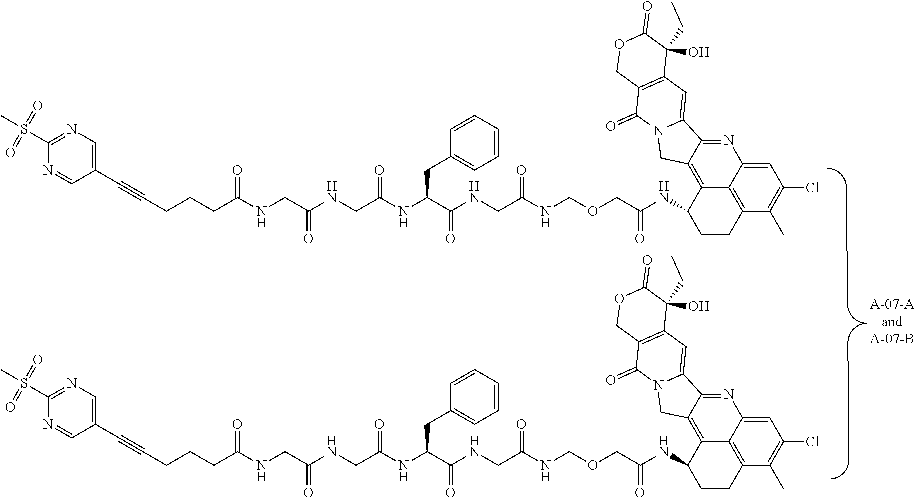

### Step 1: Synthesis of(S)-10-benzyl-23-(2-(methanesulfonyl)pyrimidin-5-yl)-6,9,12,15,18-pentaoxo-3-oxa-5,8,11,14,17-pentaazatricosane-22-yne carboxylic acid (A-07-3)

The compound A-07-2 (30.00 mg, 0.07 mmol) was dissolved in DMF (0.2 mL) at 25 °C; 2,5-dioxopyrrolidin-1-yl-6-(2-(methanesulfonyl)pyrimidin-5-yl)hexyn-5-oate ester (A-07-1, 28.00 mg, 0.08 mmol) was added, and reacted at 30 °C for 1 h; and the reaction was monitored by high-performance liquid chromatography-mass spectrometry. The reaction solution was directly purified by preparative high-performance liquid chromatography (conditions shown as follows), and the resulting solution was freeze-dried to obtain 20.00 mg of the title compound.

Chromatographic column: SunFire Prep C18
OBD 19 mm × 150 mm × 5.0 μm
Mobile phase A: acetonitrile; mobile phase
B: water (0.05% formic acid) phase A

| Time | Mobile phase A | Mobile phase B | Flow rate |
|---|---|---|---|
| [min] | [%] | [%] | [mL/min] |
| 0.00 | 10 | 90 | 28 |
| 2.00 | 10 | 90 | 28 |
| 18.00 | 90 | 10 | 28 |

The structural characterization data are as follows:

ESI-MS (m/z): 691.0 [M+H₂O]⁺.

### Step 2: Synthesis of N-((10S)-10-benzyl-1-(((1S,9S)-5-chloro-9-ethyl-9-hydroxy-4-methyl-10,13-dioxo-2,3,9,10,13,15-hexahydro-1H,12H-benzo[de]pyrano[3′,4′;6,7]indolizino[1,2-b]quinolin-1-yl)amino)-1,6,9, 12, 15-pentaoxo-3-oxa-5,8,11,14-tetraazahexadecan-16-yl)-6-(2-(methanesulfonyl)pyrimidin-5-yl)-hexan-5-amide and N-((10S)-10-benzyl-1-(((1R,9S)-5-chloro-9-ethyl-9-hydroxy-4-methyl-10,13-dioxo-2,3,9,10,13,15-hexahydro-1H,12H-benzo[de]pyrano[3′,4′;6,7]indolizino[1,2-b]quinolin-1-yl)amino)-1,6,9,12,15-pentoxo-3-oxa-5,8,11,14-tetraazahexadecan-16-yl)-6-(2-(methanesulfonyl)pyrimidin-5-yl)-hexan-5-amide (A-07-A and A-07-B)

Hydrochloride salt of 1-2 (30.00 mg, 61.43 μmol) was dissolved in N,N-dimethylformamide (1 mL) at 25 °C; A-07-3 (49.66 mg, 73.72 μmol), HATU (35.01 mg, 92.14 μmol) and N,N-diisopropylethylamine (23.82 mg, 184.29 μmol) were added in sequence; and the reaction was kept at 25 °C for 0.5 h and was monitored by high-performance liquid chromatography-mass spectrometry. After the reaction was completed, the reaction solution was purified by preparative high-performance liquid chromatography (conditions shown as follows), and the resulting solution was freeze-dried to obtain the title compound A-07. A-07 was separated under the following purification conditions to obtain two isomers, named A-07-A (i.e., A-05) (11.04 mg, retention time of 7.5 min) and A-07-B (i.e., A-06) (19.42 mg, retention time of 8.0 min) according to the retention time.

Chromatographic column: SunFire Prep C18
OBD 19 mm × 150 mm × 5.0 μm
Mobile phase A: acetonitrile; mobile phase
B: water (0.05% formic acid)

| Time | Mobile phase A | Mobile phase B | Flow rate |
|---|---|---|---|
| [min] | [%] | [%] | [mL/min] |
| 0 | 30 | 70 | 28 |
| 3 | 30 | 70 | 28 |
| 18 | 90 | 10 | 28 |

The structural characterization data are as follows:

A-07-A (i.e., A-05):

ESI-MS (m/z): 1107.3 [M+H]⁺.

¹H NMR (400 MHz, DMSO) δ 9.10 (s, 2H), 8.66-8.63 (m, 1H), 8.51 (d, J=8.8 Hz, 1H), 8.34-8.31 (m, 1H), 8.21-8.19 (m, 1H), 8.17-8.09 (m, 2H), 8.08-8.04 (m, 1H), 7.30 (s, 1H), 7.26-7.15 (m, 5H), 6.55 (s, 1H), 5.56-5.55 (m, 1H), 5.48-5.35 (m, 2H), 5.25-5.10 (m, 2H), 4.64 (d, J=6.4 Hz, 2H), 4.45-4.44 (m, 1H), 4.06-3.98 (m, 2H), 3.77-3.52 (m, 6H), 3.41 (s, 3H), 3.25-3.12 (m, 2H), 3.03-3.00 (m, 1H), 2.83-2.72 (m, 1H), 2.58-2.56 (m, 2H), 2.48 (s, 3H), 2.33-2.30 (m, 2H), 2.21-2.13 (m, 2H), 1.91-1.76 (m, 4H), 0.87 (t, J=7.2 Hz, 3H).

A-07-B (i.e., A-06):

ESI-MS (m/z): 1107.3 [M+H]⁺.

### Example 4 (S)-7-ethyl-7-hydroxyl-14-(2-(isopropylamino)ethyl)-10,13-dihydro-11H-[1,3]dioxolo[4,5-g]pyrano[3′,4′:6,7]indolizino[1,2-b]quinolin-8,11(7H)-dione (2-2)

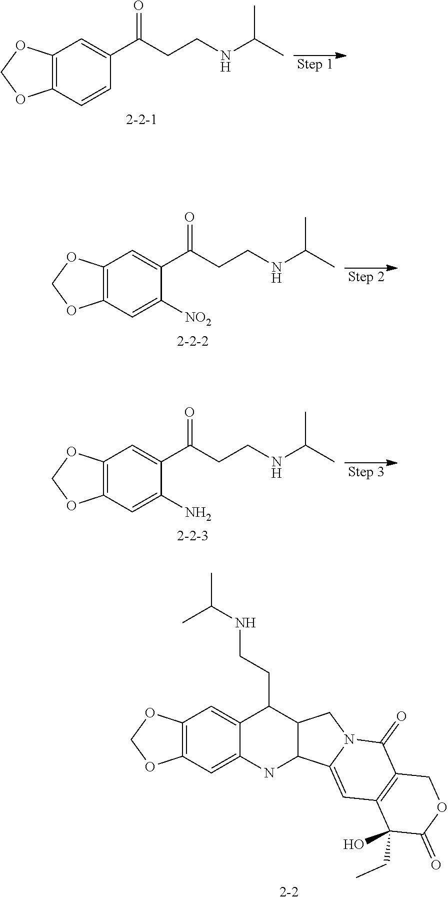 [SMILES: CC(C)NCCC(=O)c1ccc2c(c1)OCO2 ; CC(C)NCCC(=O)c1cc2c(cc1[N+](=O)[O-])OCO2 ; CC(C)NCCC(=O)c1cc2c(cc1N)OCO2 ; CC[C@@]1(O)C(=O)OCc2c1cc1n(c2=O)CC2C(CCNC(C)C)c3cc4c(cc3NC12)OCO4]

### Step 1: Synthesis of 3-(isopropylamino)-1-(6-nitrobenzo[d][1,3]dioxin-5-yl) propan-1-one (2-2-2)

The compound 2-2-1 (1.90 g, 8.08 mmol) was slowly added into nitric acid (8 mL) at 0 °C, and the temperature was slowly raised to 25 °C for reaction for 1 h. The reaction solution was directly purified by reverse-phase column chromatography (acetonitrile/0.5% aqueous formic acid) to obtain 1.50 g of the formate salt of the title compound.

The structural characterization data are as follows:

ESI-MS (m/z): 281.1 [M+H]⁺.

### Step 2: Synthesis of 1-(6-Aminobenzo[d][1,3]dioxin-5-yl)-3-(isopropylamino) propan-1-one (2-2-3)

Formate salt of the compound 2-2-2 (1.25 g, 4.46 mmol) was added into tetrahydrofuran (20 mL); 10% palladium on carbon (125.00 mg) was added; hydrogen replacement was carried out three times; and then the reaction was carried out at 25 °C for 16 h. The reaction solution was filtered through celite, and the filtrate was concentrated to dryness under reduced pressure to obtain 895.00 mg of the crude product of the title compound, which was directly used in the next reaction without purification.

The structural characterization data are as follows:

ESI-MS (m/z): 251.1 [M+H]⁺.

### Step 3: Synthesis of ((S)-7-ethyl-7-hydroxy-14-(2-(isopropylamino)ethyl)-10,13-dihydro-11H-[1,3]dioxolo[4,5-g]pyrano[3′,4′:6,7]indolizino[1,2-b]quinolin-8,11(7H)-dione (2-2)

The compound 2-2-3 (23.00 mg, 0.09 mmol) and (4S)-4-ethyl-4-hydroxy-7,8-dihydro-1H-pyrano[3,4-f]indolizine-3,6,10 (4H)-trione (21.00 mg, 0.09 mmol) were dissolved in toluene (4 mL); and p-toluenesulfonic acid (1.40 mg, 0.009 mmol) was added, and the reaction was carried out at 140 °C for 12 h. The solvent was removed under reduced pressure to obtain the crude product of the title compound; the crude product was purified by HPLC (mobile phase A: acetonitrile, mobile phase B: 0.05% of formic acid aqueous solution), and 3 drops of 3 M hydrochloric acid were added to the preparation solution, followed by freeze-drying to obtain the 8.70 mg of hydrochloride of the title compound.

The structural characterization data are as follows:

ESI-MS (m/z): 478.2 [M+H]⁺.

¹H-NMR (400 MHz, DMSO-d6): δ 9.38 (brs, 2H), 7.85 (s, 1H), 7.52 (s, 1H), 7.25 (s, 1H), 6.30 (d, J=2.0 Hz, 2H), 5.43 (s, 2H), 5.30 (s, 2H), 3.57-3.45 (m, 2H), 3.40-3.25 (m, 1H), 3.22-3.06 (m, 2H), 1.95-1.77 (m, 2H), 1.27 (d, J=6.4 Hz, 6H), 0.87 (t, J=7.6 Hz, 3H).

### Example 5 Synthesis of 4-((S)-2-(4-aminobutyl)-35-(4-((6-(2-(methanesulfonyl)pyrimidin-5-yl)hex-5-ynamido)methyl)-1H-1,2,3-triazol-1-yl)-4,8-dioxo-6,12,15,18,21,24,27,30,33-nonaoxa-3,9-diazapentatriacontanylamido)benzyl((S)-4-ethyl-11-(2-(N-isopropylmethylsulfonamido)ethyl)-3,14-dioxo-3,4,12,14-tetrahydro-1H-pyrano[3′,4′:6,7]indolizino[1,2-b]quinolin-4-yl)carbonate (B-01)

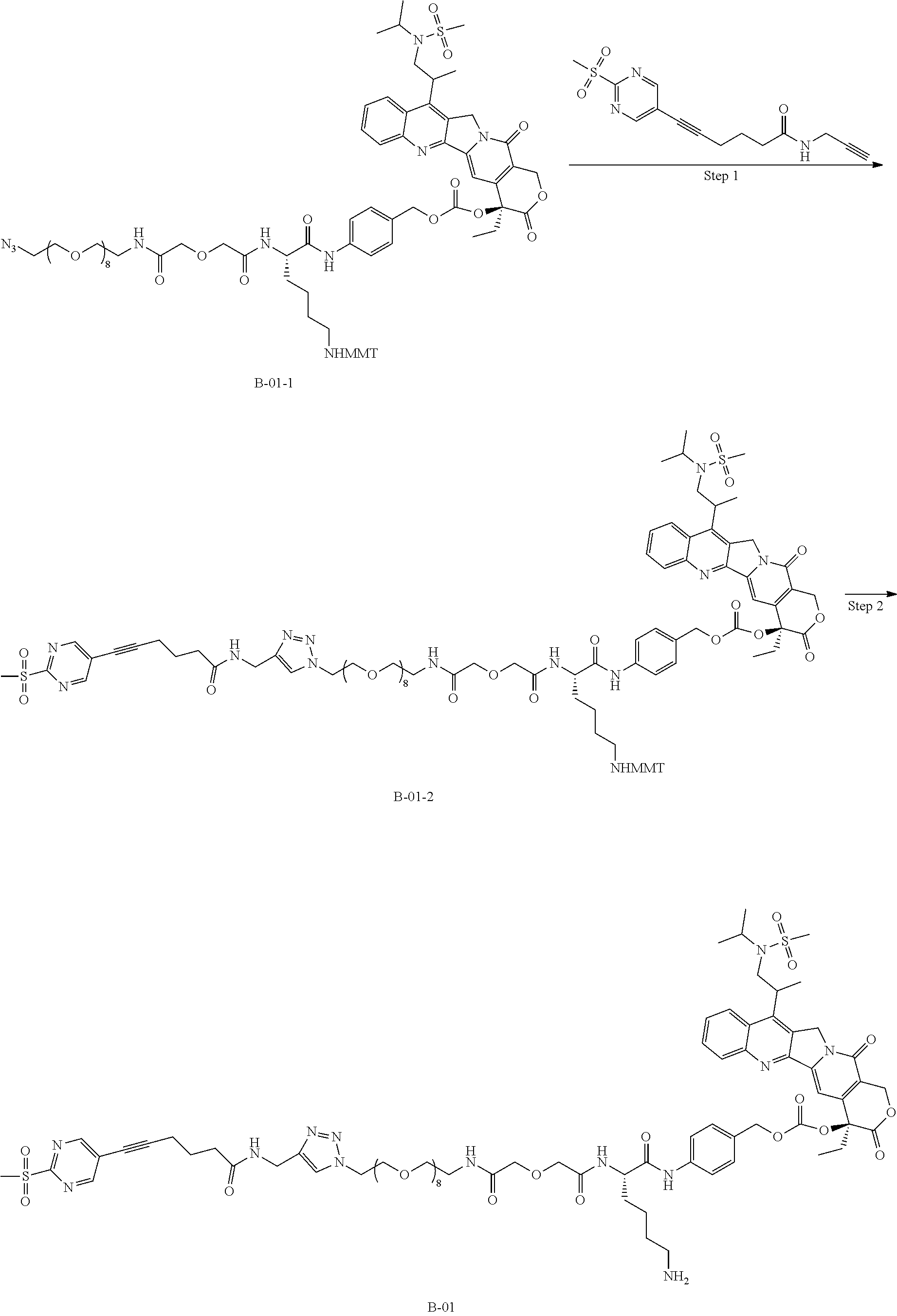

### Step 1: Synthesis of(S)-4-ethyl-11-(2-(N-isopropylmethanesulfonamido)ethyl)-3,14-dioxo-3,4, 12, 14-tetrahydro-1H-pyrano[3′,4′:6,7]indolizino[1,2-b]quinolin-4-yl(4-((S)-2-(4-(((4-methyl oxyphenyl)diphenylmethyl)amino)butyl)-35-(4-((6-(2-(methanesulfonyl)pyrimidin-5-yl)hex-5-ynamido)methyl)-1H-1,2,3-triazol-1-yl)-4,8-dioxo-6,12,15,18,21,24,27,30,33-nonaoxy-3,9-diazapentatriacontanylamido)benzyl)carbonate (B-01-2)

The compound B-01-1 (413.40 mg, 0.251 mmol, its synthesis referred to Patent CN111295389B) was dissolved in dimethyl sulfoxide and water (2.0 mL: 0.5 mL) at room temperature; cuprous bromide (72.95 mg, 0.503 mmol) and 6-(2-(methylsulfonyl)pyrimidin-5-yl)-N-(prop-2-yn-1-yl)-hex-5-ynamide (95.10 mg, 0.302 mmol) were added, and filtered after being stirred for 1 h; and the filtrate was purified by preparative high-performance liquid chromatography (conditions shown as follows) to obtain 30.00 mg of the title compound.

Chromatographic column: SunFire Prep C18
OBD 19 mm × 150 mm × 5.0 μm
Mobile phase A: acetonitrile; mobile phase B: water

| Time | Mobile phase A | Mobile phase B | Flow rate |
|---|---|---|---|
| [min] | [%] | [%] | [mL/min] |
| 0.00 | 40 | 60 | 28 |
| 8.00 | 40 | 60 | 28 |
| 50.00 | 90 | 10 | 28 |

The structural characterization data are as follows:

ESI-MS (m/z): 815.9 [(M-273)/2+H]⁺.

### Step 2: Synthesis of 4-((S)-2-(4-aminobutyl)-35-(4-((6-(2-(methylsulfonyl)pyrimidin-5-yl)hex-5-ynamido)methyl)-1H-1,2,3-triazol-1-yl)-4,8-dioxo-6,12,15,18,21,24,27,30,33-nonaoxa-3,9-diazapentatriacontanylamido)benzyl((S)-4-ethyl-11-(2-(N-isopropylmethylsulfonamido)ethyl)-3,14-dioxo-3,4,12,14-tetrahydro-1H-pyrano[3′,4′:6,7]indolizino[1,2-b]quinolin-4-yl)carbonate (B-01)

The compound B-01-3 (30.00 mg, 0.02 mmol) was dissolved in dichloromethane (1.0 mL); and trifluoroacetic acid (0.2 mL) was added to the reaction solution, and reacted at room temperature for 30 min. The reaction solution was concentrated under reduced pressure and purified by preparative high performance liquid chromatography (conditions as follows) to obtain 20.00 mg of the trifluoroacetate salt of the title compound.

Chromatographic column: SunFire Prep C18
OBD 19 mm × 150 mm × 5.0 μm
Mobile phase A: acetonitrile; mobile
phase B: water (0.05% trifluoroacetic acid)

| Time | Mobile phase A | Mobile phase B | Flow rate |
|---|---|---|---|
| [min] | [%] | [%] | [mL/min] |
| 0.00 | 15 | 85 | 28 |
| 8.00 | 15 | 85 | 28 |
| 50.00 | 60 | 40 | 28 |

The structural characterization data are as follows: ESI-MS (m/z): 508.2 [M+H]⁺.

### Example 6

### (2S,3S,4S,5R,6S)-6-(4-(((2-((S)-7-ethyl-7-hydroxy-8,11-dioxo-7,8,11,13-tetrahydro-10H-[1,3]dioxolano[4,5-g]pyrano[3′,4′:6,7]indolizino[1,2-b]quinolin-14-yl)ethyl)(isopropyl)carbamoyl)oxy)methyl)-2-(2-(2-(2-(6-(2-(methylsulfonyl)pyrimidin-5-yl)hex-5-ynamido)ethoxy)ethoxy)acetamido)phenoxy)-3,4,5-trihydroxytetrahydro-2H-pyran-2-carboxylic acid (B-03)

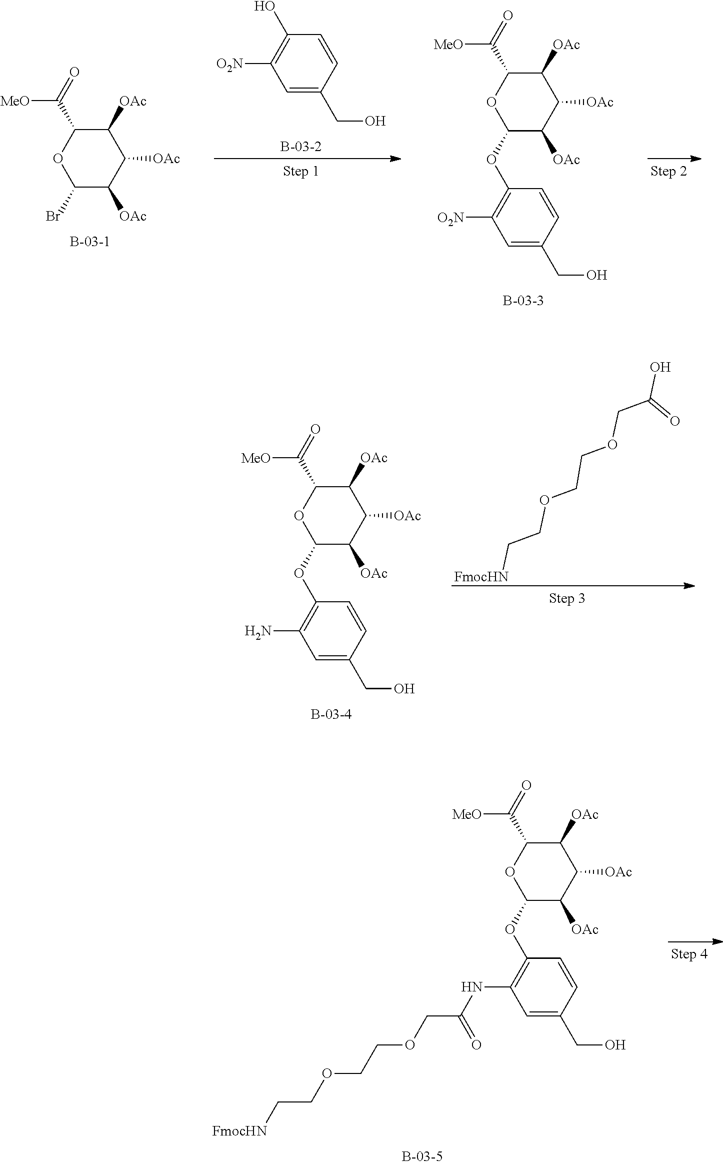

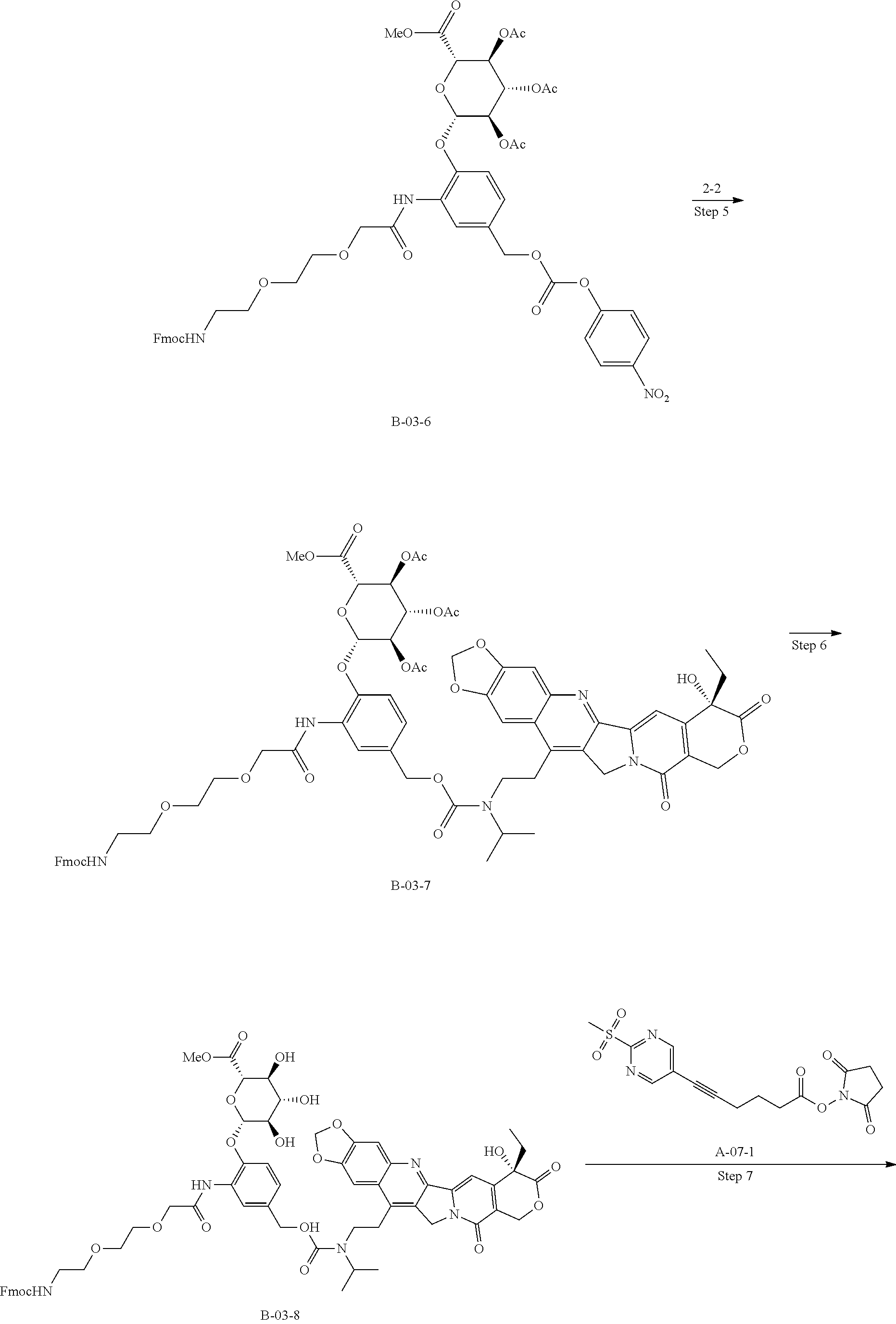

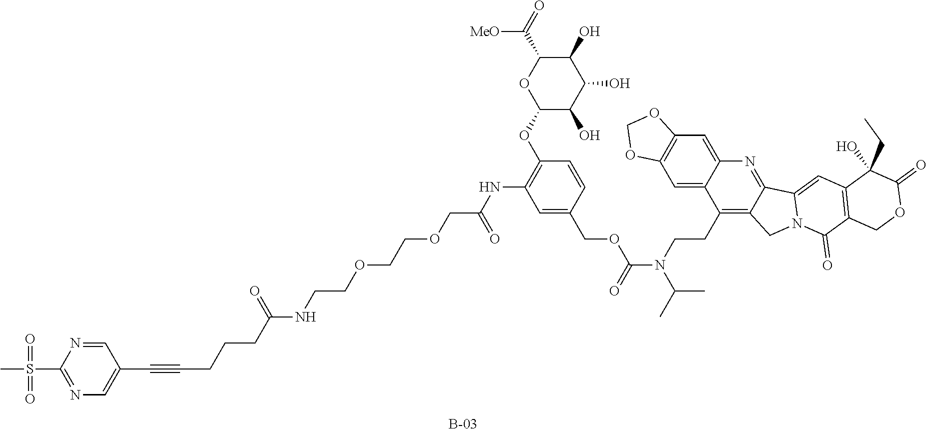 [SMILES: CC[C@@]1(O)C(=O)OCc2c1cc1n(c2=O)Cc2c-1nc1cc3c(cc1c2CCN(C(=O)OCc1ccc(O[C@@H]2O[C@H](C(=O)OC)[C@@H](O)[C@H](O)[C@H]2O)c(NC(=O)COCCOCCNC(=O)CCCC#Cc2cnc(S(C)(=O)=O)nc2)c1)C(C)C)OCO3]

### Step 1: Synthesis of (2S,3R,4S,5S,6S)-2-(4-(Hydroxymethyl)-2-nitrophenoxy)-6-(methoxycarbonyl)tetrahydro-2H-pyran-3,4,5-triacetate ester (B-03-3)

The compound (2R,3R,4S,5S,6S)-2-bromo-6-(methoxycarbonyl)tetrahydro-2H-pyran-3,4,5-triacetic acid triester (B-03-1, 12.32 g, 31.02 mmol) and 4-hydroxy-3-nitrobenzyl alcohol (compound B-03-2, 5.00 g, 29.56 mmol) were dissolved in acetonitrile (200 mL); silver oxide (27.40 g, 118.25 mmol) was added while stirring; and reaction was carried out at room temperature for 12 h in the dark after nitrogen replacement. The reaction was monitored by high-performance liquid chromatography-mass spectrometry; the reaction solution was filtered through diatomaceous earth, and the filtrate was concentrated under reduced pressure and then purified by silica gel column chromatography (petroleum ether:ethyl acetate=1:3) to obtain 12.80 g of the title compound.

The structural characterization data are as follows:

ESI-MS (m/z): 503 [M+18]⁺.

### Step 2: Synthesis of (2S,3R,4S,5S,6S)-2-(2-amino-4-(hydroxymethyl)phenoxy)-6-(methoxycarbonyl)tetrahydro-2H-pyran-3,4,5-triacetate ester (B-03-4)

The compound B-03-3 (2.20 g, 4.53 mmol) was dissolved in ethyl acetate and tetrahydrofuran (50 mL each); PtO₂ (0.20 g) was added; and then the reaction system was replaced with a hydrogen balloon three times; and reaction was carried out for 2 h under the hydrogen atmosphere. The reaction was monitored by high-performance liquid chromatography-mass spectrometry; the reaction liquid was filtered directly; and the filter cake was rinsed with ethyl acetate, and the filtrate was evaporated to dryness under reduced pressure to obtain 2.02 g of the crude product of the title compound, which was directly used in the next reaction.

The structural characterization data are as follows:

ESI-MS (m/z): 456.1 [M+1]⁺.

### Step 3: Synthesis of (2S,3R,4S,5S,6S)-2-(2-(1-(9H-fluoren-9-yl)-3-oxo-2,7,10-trioxy-4-azadodecyl-12-amino)-4-(hydroxymethyl)phenoxy)-6-(methoxycarbonyl)tetrahydro-2H-pyran-3,4,5-triacetate (B-03-5)

The compound B-03-4 (456.00 mg, 1.00 mmol) and [2-[2-(Fmoc-amino)ethoxy]ethoxy]acetic acid (385.91 mg, 1.00 mmol) were dissolved in dichloromethane (10 mL); 2-ethoxy-1-ethoxycarbonyl-1,2-dihydroquinoline (495.22 mg, 2.00 mmol) was added while stirring; and the reaction was stirred for 2 h. The reaction was monitored by high performance liquid chromatography mass spectrometry; and the reaction solution was concentrated under reduced pressure and then purified by silica gel column chromatography (methanol:dichloromethane=1:20) to obtain 507.00 mg of the title compound.

The structural characterization data are as follows:

ESI-MS (m/z): 823.3 [M+1]⁺.

Step 4: Synthesis of (2S,3R,4S,5S,6S)-2-(2-(1-(9H-fluoren-9-yl)-3,11-dioxo-2,7,10-trioxa-4,12-diazadodecyl)-4-((((4-nitrophenoxy)carbonyl)oxy)methyl)phenoxy)-6-(methoxycarbonyl)tetrahydro-2H-pyran-3,4,5-triacetate (B-03-6)

The compound B-03-5 (507.00 mg, 616.18 μmol) and diisopropylethylamine (238.91 mg, 1.85 mmol) were dissolved in dichloromethane (20 mL); then p-nitrophenyl chloroformate (372.60 mg, 1.85 mmol) was dissolved in dichloromethane (1 mL), and slowly added into the reaction solution dropwise; and after the addition was finished, the reaction was carried out at room temperature for 15 h. The reaction was monitored by high-performance liquid chromatography-mass spectrometry; and the reaction solution was concentrated under reduced pressure and then purified by silica gel column chromatography (methanol:dichloromethane=1:20) to obtain 496.00 mg of the title compound.

The structural characterization data are as follows:

ESI-MS (m/z): 988.5 [M+1]⁺.

### Step 5: Synthesis of (2S,3R,4S,5S,6S)-2-(2-(1-(9H-fluoren-9-yl)-3-oxo-2,7,10-trioxy-4-azadodecanoylamido-12-amino)-4-((((2-((S)-7-ethyl-7-hydroxy-8,11-dioxy-7,8,11,13-tetrahydro-10H-[1,3]dioxolano[4,5-g]pyrano[3′,4′:6,7]indolizino[1,2-b]quinolin-14-yl)ethyl)(isopropyl)carbamoyl)oxy)methyl)phenoxy)-6-(methoxycarbonyl)tetrahydro-2H-pyran-3,4,5-triacetate (B-03-7)

The compound B-03-6 (165.51 mg, 0.17 mmol), a compound 2-2 (40.00 mg, 0.084 mmol) and 1-hydroxybenzotriazole (33.96 mg, 0.25 mmol) were dissolved in DMF (4 mL); diisopropylethylamine (32.48 mg, 0.25 mmol) was added dropwise, and reacted for 12 under stirring; and the reaction was monitored by high-performance liquid chromatography-mass spectrometry. Water and ethyl acetate were added and stirred, and then allowed to stand for phase separation; and the organic phase was washed with saturated brine, dried, and concentrated under reduced pressure to obtain 100.00 mg of the crude product of the title compound, which was directly used in the next reaction.

The structural characterization data are as follows:

ESI-MS (m/z): 1326.2 [M+1]⁺.

### Step 6: Synthesis of (2S,3S,4S,5R,6S)-6-(2-(2-(2-(2-aminoethoxy)ethoxy)acetamido)-4-((((2-((S)-7-ethyl-7-hydroxy-8,11-dioxy-7,8,11,13-tetrahydro-10H-[1,3]dioxolano[4,5-g]pyrano[3′,4′:6,7]indolizino[1,2-b]quinolin-14-yl)ethyl)(isopropyl)carbamoyl)oxy)methyl)phenoxy)-3,4,5-trihydroxytetrahydro-2H-pyran-2-carboxylic acid (B-03-8)

The compound B-03-7 (100.00 mg, 0.08 mmol) was dissolved in MeOH (5 mL); 1 drop of dichloromethane was added dropwise; an aqueous solution of lithium hydroxide monohydrate (15.82 mg, 0.377 mmol) (1 mL) was added dropwise, and the reaction was stirred for 2 h. The reaction was monitored by high-performance liquid chromatography-mass spectrometry; 3 N of hydrochloric acid aqueous solution was added dropwise to adjust the reaction solution to pH=4; the product was concentrated under reduced pressure and then purified by preparative high-performance liquid chromatography (conditions shown as follows); and the resulting solution was freeze-dried to obtain 27.00 mg of the title compound.

Chromatographic column: SunFire Prep C18
OBD 19 mm × 150 mm × 5.0 μm
Mobile phase A: acetonitrile; mobile phase
B: water (0.05% formic acid) phase A

| Time | Mobile phase A | Mobile phase B | Flow rate |
|---|---|---|---|
| [min] | [%] | [%] | [mL/min] |
| 0.00 | 20 | 80 | 28 |
| 2.00 | 20 | 80 | 28 |
| 18.00 | 80 | 20 | 28 |

The structural characterization data are as follows:

ESI-MS (m/z): 964.2 [M+1]⁺.

### Step 7: Synthesis of (2S,3S,4S,5R,6S)-6-(4-((((2-((S)-7-ethyl-7-hydroxy-8,11-dioxo-7,8,11,13-tetrahydro-10H-[1,3]dioxolano[4,5-g]pyrano[3′,4′:6,7]indolizino[1,2-b]quinolin-14-yl)ethyl)(isopropyl)carbamoyl)oxy)methyl)-2-(2-(2-(2-(6-(2-(methylsulfonyl)pyrimidin-5-yl)hex-5-ynamido)ethoxy)ethoxy)acetamido)phenoxy)-3,4,5-trihydroxytetrahydro-2H-pyran-2-carboxylic acid (B-03)

The compound B-03-8 (27.00 mg, 0.03 mmol) and 2,5-dioxypyrrolidin-1-yl 6-(2-(methanesulfonyl)pyrimidin-5-yl)hexyn-5-oate ester (A-07-1), 11.26 mg, 0.03 mmol) were dissolved in DMF (1 mL); diisopropylethylamine (3.62 mg, 0.03 mmol) was added dropwise while stirring, and reacted at room temperature for 4 h; and the reaction was monitored by high-performance liquid chromatography-mass spectrometry. The reaction solution was purified by preparative high-performance liquid chromatography (conditions shown as follows), and the resulting solution was freeze-dried to obtain 11.70 mg of the title compound.

Chromatographic column: SunFire Prep C18
OBD 19 mm × 150 mm × 5.0 μm
Mobile phase A: acetonitrile; mobile phase
B: water (0.05% formic acid) phase A

| Time | Mobile phase A | Mobile phase B | Flow rate |
|---|---|---|---|
| [min] | [%] | [%] | [mL/min] |
| 0.00 | 20 | 80 | 28 |
| 2.00 | 20 | 80 | 28 |
| 18.00 | 80 | 20 | 28 |

The structural characterization data are as follows:

ESI-MS (m/z): 1214.4 [M+1]⁺.

### Example 7 Synthesis of N-((1S,9S)-4-chloro-9-ethyl-5-fluoro-9-hydroxy-10,13-dioxo-2,3,9,10,13,15-hexahydro-1H, 12H-benzo[de]pyrano[3′,4:6,7]indolizino[1,2-b]quinolin-1-yl)-2-hydroxyacetamide and N-((1R,9S)-4-chloro-9-ethyl-5-fluoro-9-hydroxy-10,13-dioxo-2,3,9,10,13,15-hexahydro-1H,12H-benzo[de]pyrano[3′,4:6,7]indolizino[1,2-b]quinolin-1-yl)-2-hydroxyacetamide (1-11-A and 1-11-B)

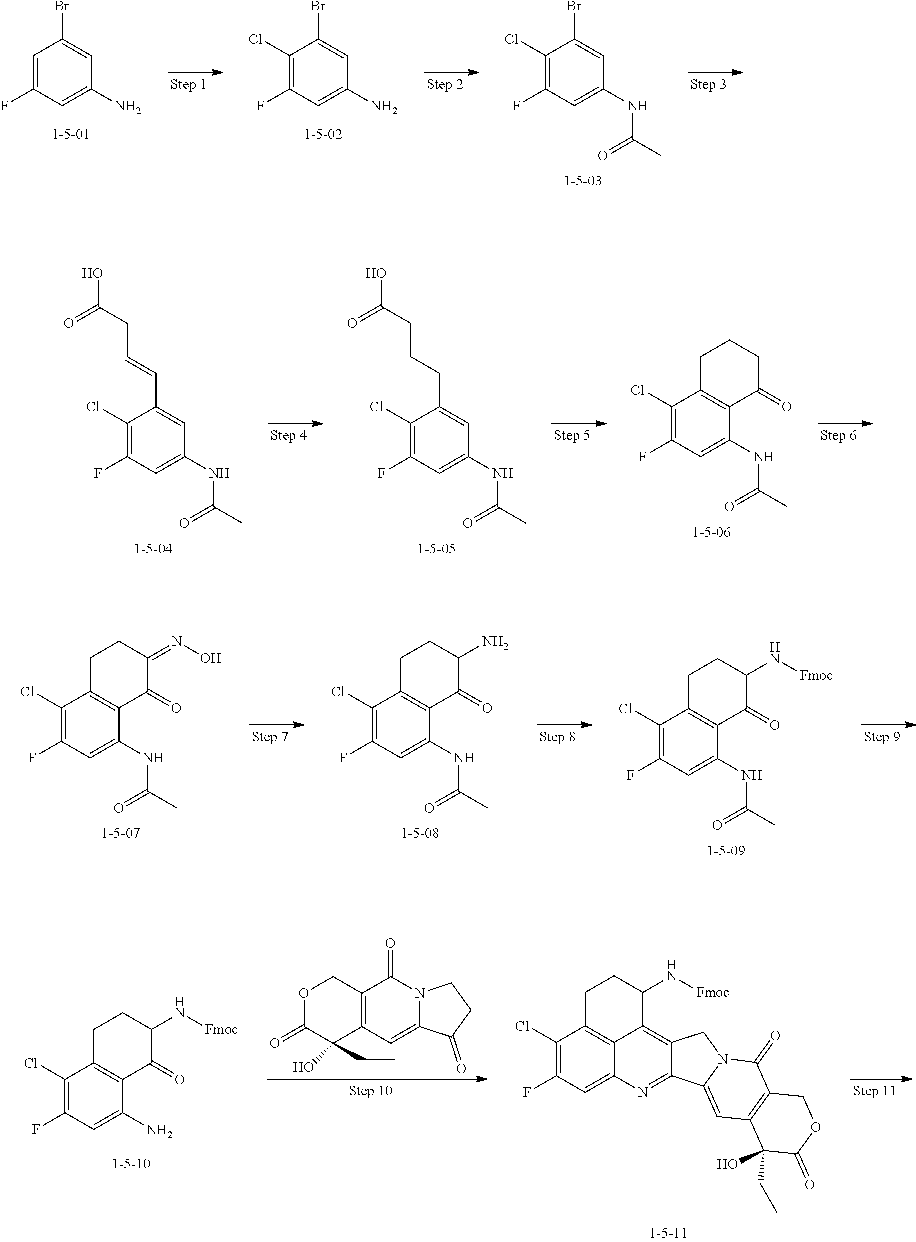

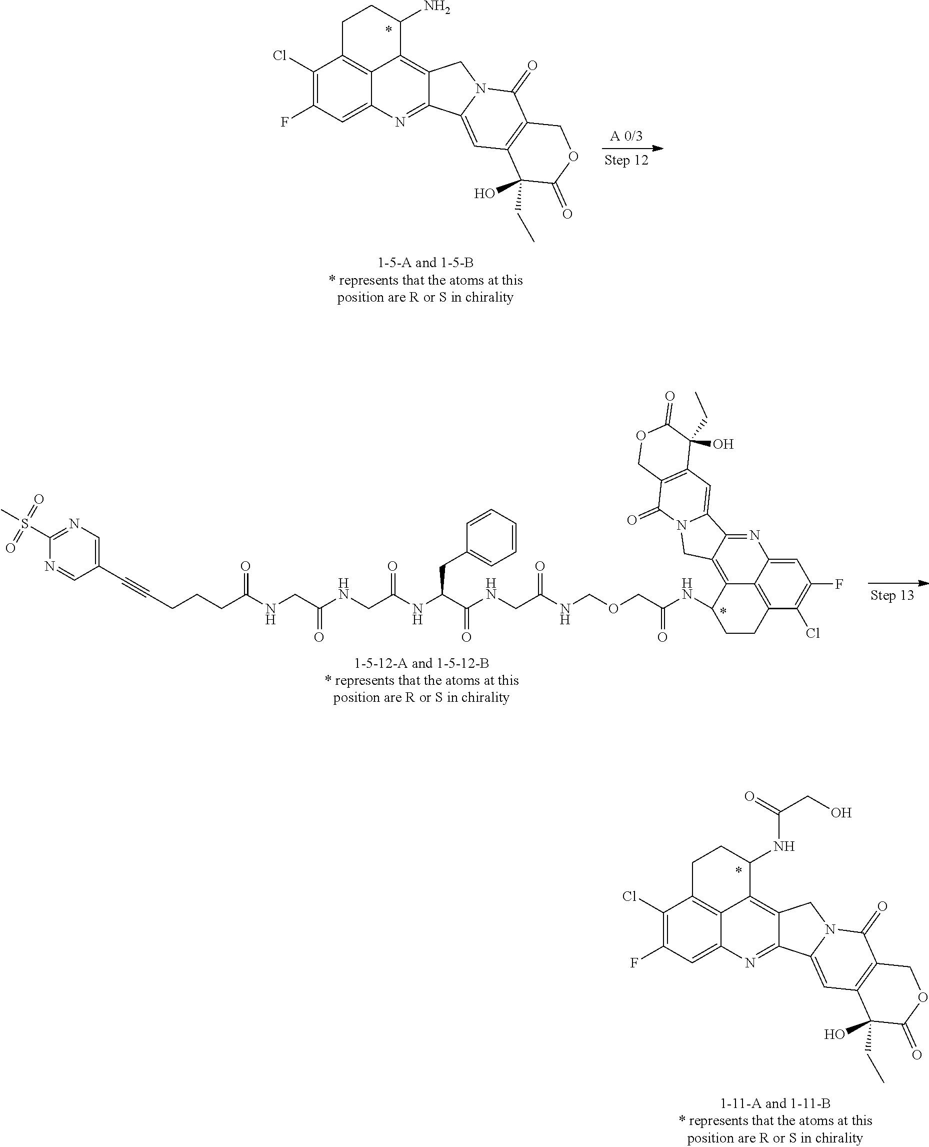 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2C(NC(=O)COCNC(=O)CNC(=O)[C@H](Cc1ccccc1)NC(=O)CNC(=O)CNC(=O)CCCC#Cc1cnc(S(C)(=O)=O)nc1)CC3 ; CC[C@@]1(O)C(=O)OCc2c1cc1n(c2=O)Cc2c-1nc1cc(F)c(Cl)c3c1c2C(NC(=O)CO)CC3]

### Step 1: Synthesis of 3-bromo-4-chloro-5-fluoroaniline (1-5-02)

The compound 1-5-01 (2.00 g, 10.53 mmol) was dissolved in N,N-dimethylformamide (30 mL); then N-chlorosuccinimide (1.69 g, 12.63 mmol) was slowly added; after the addition was finished, the reaction was carried out at room temperature for 16 h; and the reaction was monitored by high-performance liquid chromatography-mass spectrometry. The reaction solution was concentrated under reduced pressure to obtain a crude product, and the crude product was purified by flash chromatography using silica gel column (ethyl acetate:petroleum ether=0-25%) to obtain 0.95 g of the title compound.

The structural characterization data are as follows:

¹H NMR (400 MHz, DMSO-d6) δ 6.77 (dd, J=2.5, 1.4 Hz, 1H), 6.51 (dd, J=11.7, 2.5 Hz, 1H), 5.84 (s, 2H).

### Step 2: Synthesis of N-(3-bromo-4-chloro-5-fluorophenyl)acetamide (1-5-03)

The compound 1-5-02 (0.95 g, 4.23 mmol) was dissolved in ethyl acetate (20 mL), and acetic anhydride (648.13 mg, 6.35 mmol) was added under nitrogen protection; after addition was finished, the temperature was raised to 50 °C for reaction for 15 h; and the reaction was monitored by high-performance liquid chromatography-mass spectrometry. The reaction solution was quenched with methanol (5 mL), and directly evaporated to dryness under reduced pressure to obtain the crude product, which was purified by flash chromatography using silica gel column (ethyl acetate:petroleum ether=0-40%) to obtain 1.01 g of the title compound. The structural characterization data are as follows:

ESI-MS (m/z): 265.9 [M+H]⁺.

### Step 3: Synthesis of (E)-4-(5-acetamido-2-chloro-3-fluorophenyl)-3-butenoic acid (1-5-04)

The compound 1-5-03 and 3-butenoic acid (387.65 mg, 4.50 mmol) were dissolved in a mixture of 1,4-dioxane (24 mL) and water (8 mL), and then N,N-diisopropylethylamine (1.45 g, 11.26 mmol), tris(o-methylphenyl) phosphorus (114.21 mg, 375.24 μmol) and palladium acetate (42.12 mg, 187.62 μmol) were added; after addition was finished, the atmosphere in reaction system was replaced with nitrogen gas, and the temperature was raised to 100 °C for reaction for 16 h under a nitrogen atmosphere; and the reaction was monitored by high-performance liquid chromatography-mass spectrometry. After the reaction solution was cooled to room temperature, 1 N of aqueous sodium hydroxide solution (60 mL) and ethyl acetate (50 mL) were added and shaken for phase separation. After the lower aqueous phase was separated out, the pH was adjusted to be about 3 with 4 mol/L hydrochloric acid aqueous solution; then ethyl acetate was added for extraction; the organic phases were combined, washed with saturated brine, dried over anhydrous sodium sulfate, and filtered; and the filtrate was evaporated to dryness under reduced pressure to obtain 1.00 g of the crude product of the title compound.

The structural characterization data are as follows:

ESI-MS (m/z): 272.0 [M+H]⁺.

### Step 4: Synthesis of 4-(5-acetamido-2-chloro-3-fluorophenyl)butyric acid (1-5-05)

The crude product (1.00 g, 3.68 mmol) of the compound 1-5-04 was dissolved in tetrahydrofuran (15 mL); then 10% palladium on carbon (0.10 g) was added; after the addition was finished, the reaction system was replaced with a hydrogen balloon three times, and the reaction was carried out under hydrogen atmosphere for 4 h; and the reaction was monitored by high-performance liquid chromatography-mass spectrometry. The reaction solution was filtered, and the filtrate was concentrated to dryness under reduced pressure to obtain 1.00 g of the crude product of the title compound.

The structural characterization data are as follows:

ESI-MS (m/z): 274.0 [M+H]⁺.

### Step 5: Synthesis of N-(4-chloro-3-fluoro-8-oxo-5,6,7,8-tetrahydronaphthalen-1-yl)acetamide (1-5-06)

The crude product of the compound 1-5-05 (1.00 g, 3.65 mmol) was dissolved in trifluoroacetic acid (5 mL); after cooling to 5 °C, trifluoroacetic anhydride (3.84 g, 18.27 mmol, 2.54 mL) was slowly added; after the addition was finished, the temperature was kept at 5 °C for reaction for 2 h; and the reaction was monitored by high-performance liquid chromatography-mass spectrometry. The reaction solution was slowly poured into water, and then extracted with ethyl acetate; the organic phases were combined, washed with saturated brine, dried over anhydrous sodium sulfate, and then filtered; and the filtrate was evaporated to dryness under reduced pressure to obtain a crude product, which was purified by flash chromatography using silica gel column to obtain 0.43 g of the title compound.

The structural characterization data are as follows:

ESI-MS (m/z): 256.1 [M+H]⁺.

### Step 6: Synthesis of N-(4-chloro-3-fluoro-7-(hydroxyimino)-8-oxo-5,6,7,8-tetrahydronaphthalene-1-yl)acetamide (1-5-07)

Tetrahydrofuran (16 mL) and tert-butanol (4 mL) were added to the reaction solution bottle; and after cooling to 5 °C in an ice bath, potassium tert-butoxide (415.18 mg, 3.70 mmol) was added; the compound 1-5-06 (0.43 mg, 1.68 mmol) was dissolved in tetrahydrofuran (1 mL), and was added into the reaction solution dropwise; after 10 min, isoamyl nitrite (315.24 mg, 2.69 mmol) was additionally added; after the addition was finished, the temperature was kept at 5 °C for reaction for 1h; and the reaction was monitored by high-performance liquid chromatography-mass spectrometry. The reaction solution was quenched with saturated ammonium chloride aqueous solution, and extracted with ethyl acetate; the organic phases were combined, washed with saturated brine, dried over anhydrous sodium sulfate, then filtered; and the filtrate was concentrated under reduced pressure to obtain 455.00 mg of the crude product of the title compound.

The structural characterization data are as follows:

ESI-MS (m/z): 285.0 [M+H]⁺.

### Step 7: Synthesis of N-(7-amino-4-chloro-3-fluoro-8-oxo-5,6,7,8-tetrahydronaphthalen-1-yl)acetamide (1-5-08)

The crude product of the compound 1-5-07 (0.40 g, 1.41 mmol) was dissolved in methanol (10 mL); then 3 mol/L of aqueous hydrochloric acid (1 mL) and 10% of palladium on carbon (40.00 mg) were added; after the addition was finished, the atmosphere in reaction system was replaced with hydrogen gas; the reaction was conducted at room temperature under a hydrogen atmosphere for 1 h, and was monitored by high-performance liquid chromatography-mass spectrometry. The reaction solution was filtered, and the filtrate was concentrated under reduced pressure to obtain 0.43 mg of crude hydrochloride salt of the title compound.

The structural characterization data are as follows:

ESI-MS (m/z): 271.0 [M+H]⁺.

### Step 8: Synthesis of (9H-fluoren-9-yl)methyl(8-acetamido-5-chloro-6-fluoro-1-oxo-1,2,3,4-tetrahydronaphthalen-2-yl)carbamate (1-5-09)

The crude hydrochloride salt of the compound 1-5-08 (0.43 g, 1.19 mmol) was dissolved in 1,4-dioxane (15 mL); then sodium bicarbonate (400.35 mg, 4.77 mmol), water (5 mL) and 9-fluorenylmethyl-N-succinimidyl carbonate (481.81 mg, 1.43 mmol) were added; after the addition was finished, the reaction was carried out at room temperature for 2 h while stirring; and the reaction was monitored by high-performance liquid chromatography-mass spectrometry. The reaction solution was poured into water, and then extracted with ethyl acetate; the organic phases were combined, washed with saturated brine, dried over anhydrous sodium sulfate, and filtered; and the filtrate was concentrated under reduced pressure to obtain a crude product. The crude product was purified by a C18 reverse-phase column (acetonitrile: 0.05% formic acid in water=20%-100%) to obtain 301.00 mg of the title compound.

The structural characterization data are as follows:

ESI-MS (m/z): 493.2 [M+H]⁺.

### Step 9: Synthesis of (9H-fluoren-9-yl)methyl(8-amino-5-chloro-6-fluoro-1-oxo-1,2,3,4-tetrahydronaphthalen-2-yl)carbamate (1-5-10)

The compound 1-5-09 (300.00 mg, 608.61 μmol) was dissolved in dioxane (5 mL); 12 mol/L concentrated hydrochloric acid (1 mL) was added; after the addition was finished, the temperature was raised to 60 °C for reaction for 2 h; and the reaction was monitored by high-performance liquid chromatography-mass spectrometry. The reaction solution was poured into water, and then extracted with ethyl acetate; the organic phases were combined, washed with saturated brine, dried over anhydrous sodium sulfate, and filtered; and the filtrate was concentrated under reduced pressure to obtain a crude product. The crude product was purified by flash chromatography using silica gel column (ethyl acetate:petroleum ether=0-50%) to obtain 198.00 mg of the title compound.

The structural characterization data are as follows:

ESI-MS (m/z): 451.1 [M+H]⁺.

### Step 10: Synthesis of (9H-fluoren-9-yl)methyl((9S)-4-chloro-9-ethyl-5-fluoro-9-hydroxyl-10,13-dioxy-2,3,9,10,13,15-hexahydro-1H,12H-benzo[de]pyrano[3′,4:6,7]indolizino[1,2-b]quinolin-1-yl)carbamate (1-5-11)

(S)-4-ethyl-4-hydroxyl-7,8-dihydro-1H-pyrano[3,4-f]indolizin-3,6,10 (4H)-trione (138.72 mg, 526.96 μmol) and the compound 1-5-10 (198.00 mg, 439.13 μmol) were added in toluene (10 mL); then p-toluenesulfonic acid (75.53 mg, 439.13 μmol) was added; after addition was finished, the temperature was raised to 140 °C for reaction for 4 h; the reaction solution was directly evaporated to dryness at 140 °C under reduced pressure to obtain a crude product, which was purified by flash chromatography using silica gel column (methanol:dichloromethane=0-5%) to obtain 256.00 mg of the title compound.

The structural characterization data are as follows:

ESI-MS (m/z): 678.1 [M+H]⁺.

### Step 11: Synthesis of (1S,9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinolin-10,13-dione and (1R,9S)-1-amino-4-chloro-9-ethyl-5-fluoro-9-hydroxy-1,2,3,9,12,15-hexahydro-10H,13H-benzo[de]pyrano[3′,4′:6,7]indolizino[1,2-b]quinolin-10,13-dione (1-5-A and 1-5-B)

The compound 1-5-11 (201.18 mg, 296.67 μmol) was dissolved in N,N-dimethylformamide (4 mL); then diethylamine (108.49 mg, 1.48 mmol) was added; after the addition was finished, the reaction was carried out at room temperature for 0.5 h; and the reaction was monitored by high-performance liquid chromatography-mass spectrometry. After the ethylenediamine was distilled off from reaction solution under reduced pressure, the pH was adjusted to 2-3 with 1 mol/L hydrochloric acid aqueous solution; and the reaction solution was directly purified by preparative high performance liquid chromatography to obtain the title compounds 1-5-A (44.00 mg), and 1-5-B (43.00 mg).

Chromatographic column: SunFire Prep C18
OBD 19 mm × 150 mm × 5.0 μm
Mobile phase A: acetonitrile; mobile phase
B: water (0.05% formic acid)

| Time | Mobile phase A | Mobile phase B | Flow rate |
|---|---|---|---|
| [min] | [%] | [%] | [mL/min] |
| 0 | 10 | 90 | 28 |
| 3 | 10 | 90 | 28 |
| 18 | 70 | 30 | 28 |

1-5-A (6 min LCMS peak comes first, retention time: 1.276 min)

The structural characterization data are as follows:

¹H NMR (400 MHz, DMSO-d6) δ 8.00 (d, J=10.3 Hz, 1H), 7.33 (s, 1H), 6.54 (s, 1H), 5.62 (d, J=19.3 Hz, 1H), 5.44 (s, 2H), 5.38 (d, J=19.3 Hz, 1H), 4.43-4.38 (m, 1H), 3.28-3.10 (m, 2H), 2.22-2.12 (m, 1H), 2.12-2.02 (m, 1H), 1.93-1.80 (m, 2H), 0.87 (t, J=7.3 Hz, 3H).

ESI-MS (m/z): 456.1 [M+H]⁺.

The structural characterization data of 1-5-B (6 min LCMS peak comes later, retention time: 1.300 min) are as follows:

¹H NMR (400 MHz, DMSO-d6) δ 7.98 (d, J=10.3 Hz, 1H), 7.32 (s, 1H), 5.61 (d, J=19.4 Hz, 1H), 5.44 (s, 2H), 5.32 (d, J=19.4 Hz, 1H), 4.44-4.36 (m, 1H), 3.33-3.25 (m, 1H), 3.22-3.11 (m, 1H), 2.23-2.13 (m, 1H), 2.11-2.03 (m, 1H), 1.96-1.82 (m, 2H), 0.89 (t, J=7.3 Hz, 3H).

ESI-MS (m/z): 456.1 [M+H]⁺.

6 min LCMS conditions:

Chromatographic column: Waters SunFire C18
OBD 4.6 mm × 50 mm × 5.0 μm
Mobile phase A: 0.05% acetonitrile; mobile
phase B: water (0.05% formic acid)

| Time | Mobile phase A | Mobile phase B | Flow rate |
|---|---|---|---|
| [min] | [%] | [%] | [mL/min] |
| 0 | 90 | 10 | 2 |
| 4.2 | 10 | 90 | 2 |
| 5.7 | 10 | 90 | 2 |
| 5.71 | 90 | 10 | 2 |
| 6.70 | 90 | 10 | 2 |

### Step 12: Synthesis of N-((10S)-10-benzyl-1-(((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolazino[1,2-b]quinolin-1-yl)amino)-1,6,9,12,15-pentaoxo-3-oxa-5,8,11,14-tetraazahexadecan-16-yl)-6-(2-(methylsulfonyl)pyrimidin-5-yl)hex-5-ynamide and N-((10S)-10-benzyl-1-(((1R,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-yl)amino)-1,6,9,12,15-pentoxo-3-oxa-5,8,11, 14-tetraazahexadecan-16-yl)-6-(2-(methylsulfonyl)pyrimidin-5-yl)hex-5-ynamide (1-5-12-A and 1-5-12-B)

The compound 1-5-A (36.00 mg, 79.70 μmol) and the compound A-07-3 (64.43 mg, 95.64 μmol) in single configuration were dissolved in N,N-dimethylformamide (2 mL); then 4-(4,6-dimethoxytriazin-2-yl)-4-methylmorpholine hydrochloride (46.98 mg, 159.40 μmol) and triethylamine (24.19 mg, 239.10 μmol) were added; after addition was finished, reaction was carried out at room temperature for 1 h; and the reaction was monitored by high-performance liquid chromatography-mass spectrometry. The reaction solution was directly purified by high performance liquid chromatography to obtain the title compound 1-5-12-A (51.00 mg, A-14) in single configuration.

Chromatographic column: SunFire Prep C18
OBD 19 mm × 150 mm × 5.0 μm
Mobile phase A: acetonitrile; mobile phase
B: water (0.05% formic acid) phase A

| Time | Mobile phase A | Mobile phase B | Flow rate |
|---|---|---|---|
| [min] | [%] | [%] | [mL/min] |
| 0 | 30 | 70 | 28 |
| 3 | 30 | 70 | 28 |
| 18 | 90 | 10 | 28 |

The structural characterization data are as follows:

ESI-MS (m/z): 1111.0 [M+H]⁺.

The compound 1-5-B (36.00 mg, 79.70 μmol) and the compound A-07-3 (64.43 mg, 95.64 μmol) in single configuration were dissolved in N,N-dimethylformamide (2 mL); then 4-(4,6-dimethoxytriazin-2-yl)-4-methylmorpholine hydrochloride (46.98 mg, 159.40 μmol) and triethylamine (24.19 mg, 239.10 μmol) were added; after addition was finished, reaction was carried out at room temperature for 1 h; and the reaction was monitored by high-performance liquid chromatography-mass spectrometry. The reaction solution was directly purified by high-performance liquid chromatography to obtain the title compound 1-5-12-B (52.00 mg, A-15) in single configuration.

Chromatographic column: SunFire Prep C18
OBD 19 mm × 150 mm × 5.0 μm
Mobile phase A: acetonitrile; mobile
phase B: water (0.05% formic acid)

| Time | Mobile phase A | Mobile phase B | Flow rate |
|---|---|---|---|
| [min] | [%] | [%] | [mL/min] |
| 0 | 30 | 70 | 28 |
| 3 | 30 | 70 | 28 |
| 18 | 90 | 10 | 28 |

The structural characterization data are as follows:

ESI-MS (m/z): 1111.0 [M+H]⁺.

Step 13: Synthesis of N-((1S,9S)-4-chloro-9-ethyl-5-fluoro-9-hydroxy-10,13-dioxo-2,3,9,10,13,15-hexahydro-1H,12H-benzo[de]pyrano[3′,4:6,7]indolizino[1,2-b]quinolin-1-yl)-2-hydroxyacetamide and N-((1R,9S)-4-chloro-9-ethyl-5-fluoro-9-hydroxy-10,13-dioxo-2,3,9,10,13,15-hexahydro-1H,12H-benzo[de]pyrano[3′,4:6,7]indolizino[1,2-b]quinolin-1-yl)-2-hydroxyacetamide (1-11-A and 1-11-B)

The compound 1-5-12-A (40.00 mg, 35.99 μmol) was weighed and dissolved in a mixture of dichloromethane (2 mL) and methanol (1 mL); then 4 mol/L of ethyl acetate hydrochloride solution (1 mL) was added; after the addition was finished, reaction was carried out at room temperature for 0.5 h; and the reaction was monitored by high-performance liquid chromatography-mass spectrometry. The reaction solution was directly concentrated to dryness under reduced pressure to obtain a crude product, which was purified by high-performance liquid chromatography to obtain the title compound 1-11-A (4.75 mg) in single configuration.

Chromatographic column: SunFire Prep C18
OBD 19 mm × 150 mm × 5.0 μm
Mobile phase A: acetonitrile; mobile phase
B: water (0.05% formic acid) phase A

| Time | Mobile phase A | Mobile phase B | Flow rate |
|---|---|---|---|
| [min] | [%] | [%] | [mL/min] |
| 0 | 15 | 85 | 28 |
| 3 | 15 | 85 | 28 |
| 18 | 90 | 10 | 28 |

The structural characterization data are as follows:

¹H NMR (400 MHz, DMSO-d6) δ 8.50 (d, J=8.9 Hz, 1H), 8.05 (d, J=10.3 Hz, 1H), 7.33 (s, 1H), 6.55 (s, 1H), 5.67-5.60 (m, 1H), 5.49 (t, J=5.8 Hz, 1H), 5.43 (s, 2H), 5.21 (s, 2H), 3.96 (d, J=5.8 Hz, 2H), 3.32-3.22 (m, 2H), 2.28-2.15 (m, 2H), 1.93-1.80 (m, 2H), 0.87 (t, J-7.3 Hz, 3H).

ESI-MS (m/z): 514.0 [M+H]⁺.

The compound 1-5-12-B (40.00 mg, 35.99 μmol) was weighed and dissolved in a mixture of dichloromethane (2 mL) and methanol (1 mL); then 4 mol/L of ethyl acetate hydrochloride (1 mL) was added; after the addition was finished, reaction was carried out at room temperature for 0.5 h; and the reaction was monitored by high-performance liquid chromatography-mass spectrometry. The reaction solution was directly concentrated to dryness under reduced pressure to obtain a crude product, which was purified by high-performance liquid chromatography to obtain the single configuration of the title compound 1-11-B (8.24 mg).

Chromatographic column: SunFire Prep C18
OBD 19 mm × 150 mm × 5.0 μm
Mobile phase A: acetonitrile; mobile
phase B: water (0.05% formic acid)

| Time | Mobile phase A | Mobile phase B | Flow rate |
|---|---|---|---|
| [min] | [%] | [%] | [mL/min] |
| 0 | 15 | 85 | 28 |
| 3 | 15 | 85 | 28 |
| 18 | 90 | 10 | 28 |

The structural characterization data are as follows:

¹H NMR (400 MHz, DMSO-d6) δ 8.52 (d, J=9.0 Hz, 1H), 8.05 (d, J=10.3 Hz, 1H), 7.34 (s, 1H), 6.55 (s, 1H), 5.68-5.58 (m, 1H), 5.53 (t, J=5.8 Hz, 1H), 5.43 (d, J=2.9 Hz, 2H), 5.20 (d, J=7.3 Hz, 2H), 3.97 (d, J=5.7 Hz, 2H), 3.31-3.21 (m, 2H), 2.26-2.15 (m, 2H), 1.92-1.82 (m, 2H), 0.87 (t, J=7.3 Hz, 3H).

ESI-MS (m/z): 514.0 [M+H]⁺.

### Example 8 N-((10S,19S)-amino-10-benzyl-1-((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-amino)-1,6,9,12,15,18,25-heptaoxo-3-oxa-5,8,11,14,17,24-hexaaza-30-(2-methylsulfonylpyrimidin-5-yl)-29-triacontyn-19-yl)-2,5,8,11,14,17,20,23,26,29,32,35-dodecoxaoctatriacontan-38-amide (A-17-A)

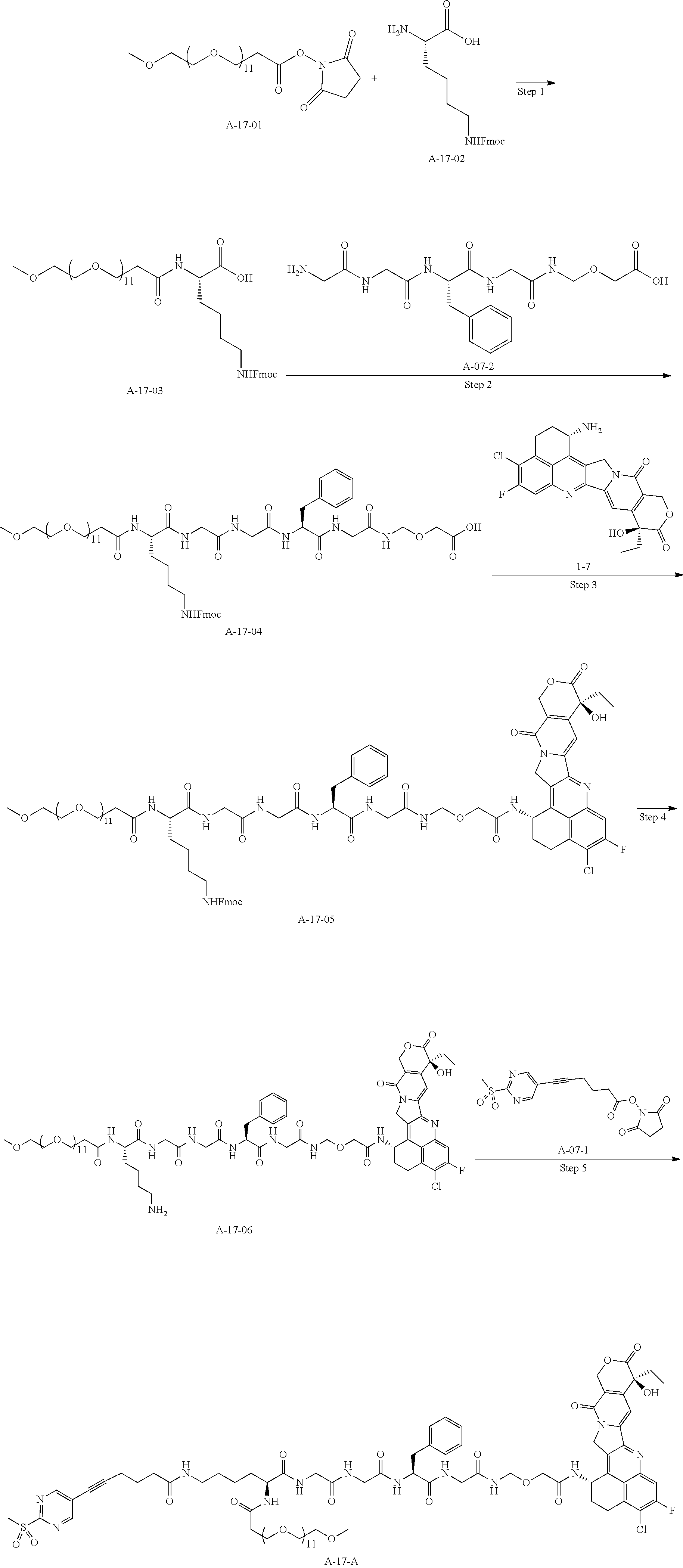

### Step 1: Synthesis of (2S)-2-(2,5,8,11,14,17,20,23,26,29,32,35-dodecoxaoctatriacontan-38-amido)-6-({[(9H-fluoren-9-yl) methoxy]carbonyl}amino)hexanoic acid (A-17-03)

Hydrochloride salt of the compound A-17-02 (389.68 mg, 962.45 μmol) was dissolved in dichloromethane (8 mL), and DIPEA (518.28 mg, 4.01 mmol, 713.88 μL) and the compound A-17-01 (550.00 mg, 802.04 μmol) were added, and reacted at 25 °C for 1.5 h. The pH of the reaction solution was adjusted to neutral with dilute hydrochloric acid, and the solvent was removed under reduced pressure. The concentrate was purified by preparative high performance liquid chromatography to obtain the title compound A-17-03 (450.00 mg).

Chromatographic column: SunFire Prep C18
OBD 19 mm × 150 mm × 5.0 μm
Mobile phase A: acetonitrile; mobile phase
B: water (0.05% formic acid)

| Time | Mobile phase A | Mobile phase B | Flow rate |
|---|---|---|---|
| [min] | [%] | [%] | [mL/min] |
| 0 | 10 | 90 | 28 |
| 2 | 10 | 90 | 28 |
| 18 | 80 | 20 | 28 |

ESI-MS (m/z): 939.3[M + H]⁺.

### Step 2: Synthesis of 2-({2-[(2S)-2-(2-{2-[(2S)-2-(2,5,8,11,14,17,20,23,26,29,32,35-dodecoxaoctatriacontan-38-amido)-6-({[(9H-fluoren-9-yl)methoxy]carbonyl}amino)hexanamido]acetamido}acetamido)-3-phenylpropanyl]acetamido}methoxy)acetic acid (A-17-04)

The compound A-17-03 (50.00 mg, 118.09 μmol) was dissolved in DMF (2.5 mL); and HATU (49.39 mg, 129.89 μmol), the compound A-07-2 (133.07 mg, 141.70 μmol) and DIPEA (45.78 mg, 354.26 μmol, 63.06 μL) were added, and reacted at 25 °C for 1 h. The solvent was removed under reduced pressure; and the concentrate was purified by preparative high-performance liquid chromatography to obtain the title compound A-17-04 (40.00 mg).

Chromatographic column: SunFire Prep C18
OBD 19 mm × 150 mm × 5.0 μm
Mobile phase A: acetonitrile; mobile phase
B: water (0.05% formic acid)

| Time | Mobile phase A | Mobile phase B | Flow rate |
|---|---|---|---|
| [min] | [%] | [%] | [mL/min] |
| 0 | 10 | 90 | 28 |
| 2 | 10 | 90 | 28 |
| 18 | 80 | 20 | 28 |

ESI-MS (m/z): 1344.4[M + H]⁺.

### Step 3: Synthesis of (9H-fluoren-9-yl)methyl ((40S)-40-((10S)-10-benzyl-1-((9S)-4-chloro-9-ethyl-5-fluoro-9-hydroxy-10,13-dioxo-2,3,9, 10, 13, 15-hexahydro-1H, 12H-benzo[de]pyrano[3′,4′:6,7]indolizino[1,2-b]quinolin-1-amino)-1,6,9,12,15-pentoxo-3-oxa-5,8,11,14-tetraazahexadecan-16-ylcarbamoyl)-38-oxo-2,5,8,11,14,17,20,23,26,29,35-dodecoxa-39-aza-44-yl)carbamate (A-17-05)

The compound 1-5-04 (29.86 mg, 59.50 μmol) was dissolved in DMF (3 mL); and HATU (27.15 mg, 71.40 μmol), and the compound 1-7 (80.00 mg, 59.50 μmol) and DIPEA (38.45 mg, 297.51 μmol, 52.96 μL) were added, and reacted at 25 °C for 1 h. The solvent was removed under reduced pressure; and the concentrate was purified by preparative high-performance liquid chromatography to obtain the title compound A-17-05 (50.00 mg).

Chromatographic column: SunFire Prep C18
OBD 19 mm × 150 mm × 5.0 μm
Mobile phase A: acetonitrile; mobile phase
B: water (0.05% formic acid)

| Time | Mobile phase A | Mobile phase B | Flow rate |
|---|---|---|---|
| [min] | [%] | [%] | [mL/min] |
| 0 | 10 | 90 | 28 |
| 2 | 10 | 90 | 28 |
| 18 | 80 | 20 | 28 |

ESI-MS (m/z): 1781.6[M + H]⁺.

### Step 4: Synthesis of N-((10S,19S)-23-amino-10-benzyl-1-((9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-amino)-1,6,9,12,15,18,25-heptaoxy-3-oxo-5,8,11,14,17,24-hexaazatriacontan-19-yl)-2,5,8,11,14,17,20,23,26,29,32,35-dodecaoxatrioctadecan-38-amide (A-17-06)

The compound A-17-05 (20.00 mg, 11.22 μmol) was dissolved in DMF (2.5 mL) and diethylamine (0.5 mL), and reacted at 25 °C for 2 h. The solvent was removed under reduced pressure; and the concentrate was purified by preparative high-performance liquid chromatography to obtain formate salt of the title compound A-17-06 (10.00 mg).

Chromatographic column: SunFire Prep C18
OBD 19 mm × 150 mm × 5.0 μm
Mobile phase A: acetonitrile; mobile phase
B: water (0.05% formic acid)

| Time | Mobile phase A | Mobile phase B | Flow rate |
|---|---|---|---|
| [min] | [%] | [%] | [mL/min] |
| 0 | 15 | 85 | 28 |
| 2 | 15 | 85 | 28 |
| 18 | 80 | 20 | 28 |

ESI-MS (m/z): 1559.7[M + H]+.

### Step 5: Synthesis of N-((10S,19S)-amino-10-benzyl-1-((1S,9S)-4-chloro-9-ethyl-5-fluoro-9-hydroxy-10,13-dioxo-2,3,9,10,13,15-hexahydro-1H,12H-benzo[de]pyrano[3′,4′:6,7]indolizino[1,2-b]quinolin-1-amino)-1,6,9,12,15,18,25-heptaoxo-3-oxa-5,8,11,14,17,24-hexaaza-30-(2-methylsulfonylpyrimidin-5-yl)-29-triacontyn-19-yl)-2,5,8,11,14,17,20,23,26,29,32,35-dodecaoxatrioctadecan-38-amide (A-17-A)

The formate salt of the compound A-17-06 (7.00 mg, 4.49 μmol) and the compound A-07-1 (3.28 mg, 8.97 μmol) were dissolved in DMF (1 mL), into which DIPEA (1.74 mg, 13.46 μmol, 2.40 μL) was added, and reacted at 25 °C for 2 h. The solvent was removed under reduced pressure; and the concentrate was purified by preparative high-performance liquid chromatography to obtain the title compound A-17-A (5.60 mg).

Chromatographic column: SunFire Prep C18
OBD 19 mm × 150 mm × 5.0 μm
Mobile phase A: acetonitrile; mobile phase
B: water (0.05% formic acid)

| Time | Mobile phase A | Mobile phase B | Flow rate |
|---|---|---|---|
| [min] | [%] | [%] | [mL/min] |
| 0 | 20 | 80 | 28 |
| 2 | 20 | 80 | 28 |
| 18 | 80 | 20 | 28 |

ESI-MS (m/z): 1809.8[M + H]⁺.

### Example 9 4-((S)-2-((S)-2-(6-(2,5-dioxo-2,5-dihydro-1H-pyrrol-1-yl)hexanamido)-3-methylbutyramido)-5-ureidopentaneamido)benzyl(1-(((S)-1-(((3R,4S,5S)-3-methoxy-1-((S)-2-((1R,2R)-1-methoxy-2-methyl-3-oxo-3-(((S)-2-phenyl-1-(thiazole-2-yl)ethyl)amino)propyl)pyrrolidin-1-yl)-5-methyl-1-oxohept-4-yl)(methyl)amino)-3-methyl-1-oxobutane-2-yl)amino)-2-methyl-1-oxopropyl-2-yl)carbamate (C-01)

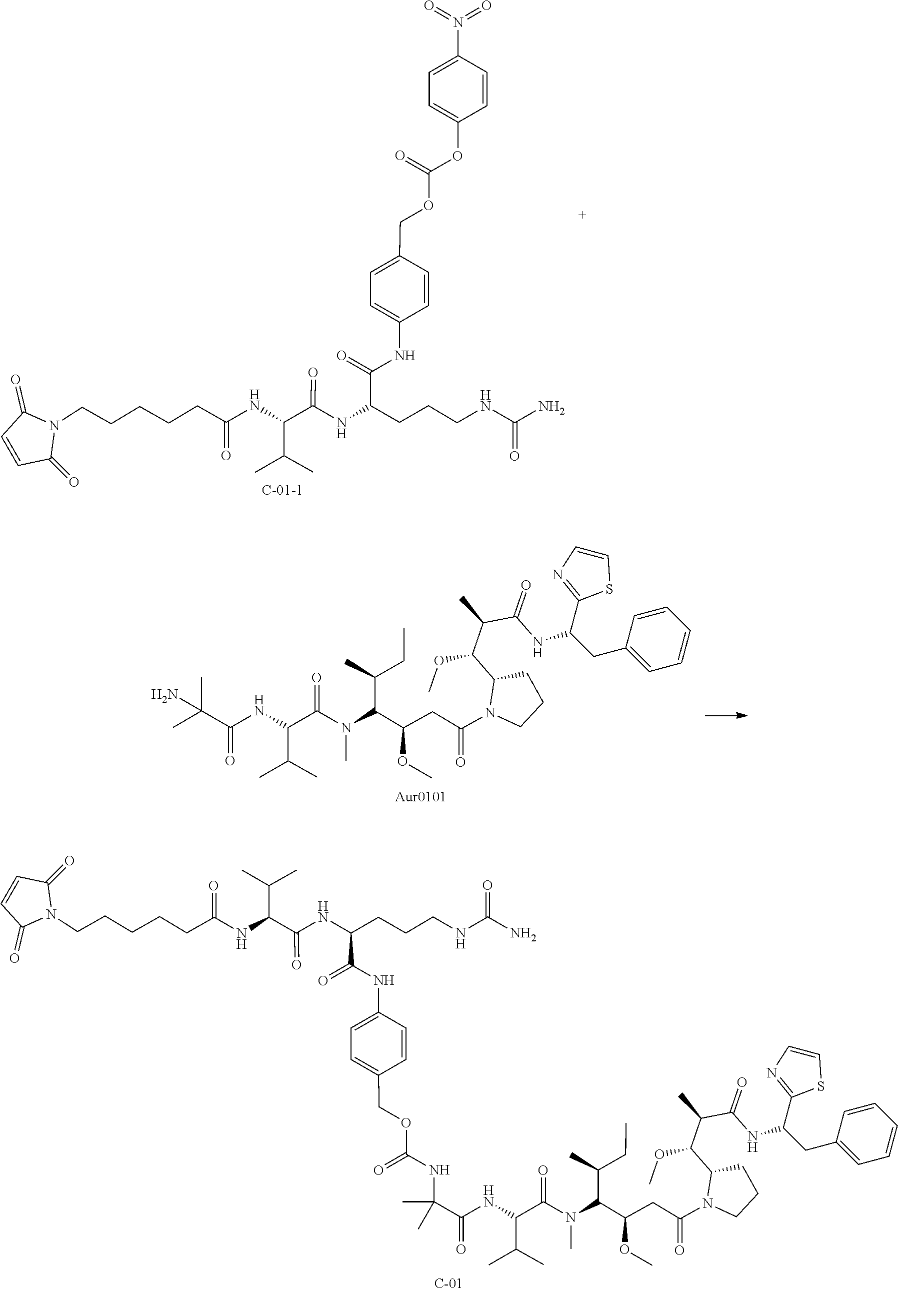

According to the synthetic procedure in International Patent Publication No. WO 2015/168019 A2, 4-((S)-2-((S)-2-(6-(2,5-dioxy-2,5-dihydro-1H-pyrrol-1-yl)hexylamino)-3-methylbutylamino)-5-ureidopentaneamido)benzyl(4-nitrophenyl)carbonate (C-01-1) and(S)-2-(2-amino-2-methylpropionamido)-N-((3R,4S,5S)-3-methoxy-1-((S)-2-((1R,2R)-1-methoxy-2-methyl-3-oxo-3-(((S)-2-phenyl-1-(thiazol-2-yl)ethyl)amino)propyl)pyrrolidin-1-yl)-5-methyl-1-oxohept-4-yl)-N,3-dimethylbutyramide (Aur0101) were used to obtain the title compound C-01.

ESI-MS (m/z): 1341.7 [M+1]⁺

### Example 10: N-((7S,10S,13S)-1-(((1S,9S)-5-chloro-9-ethyl-9-hydroxyl-4-methyl-10,13-dioxo-2,3,9,10,13,15-hexahydro-1H,12H-benzo[de]pyrano[3′,4′:6,7]indolizino[1,2-b]quinolin-1-yl)amino)-7,10-dimethyl-1,6,9,12-tetroxide-3-oxy-5,8,11-triazidetradecane-13-yl)-6-(2-(methylsulfonyl)pyrimidin-5-yl)hex-5-ynamido (A-26)

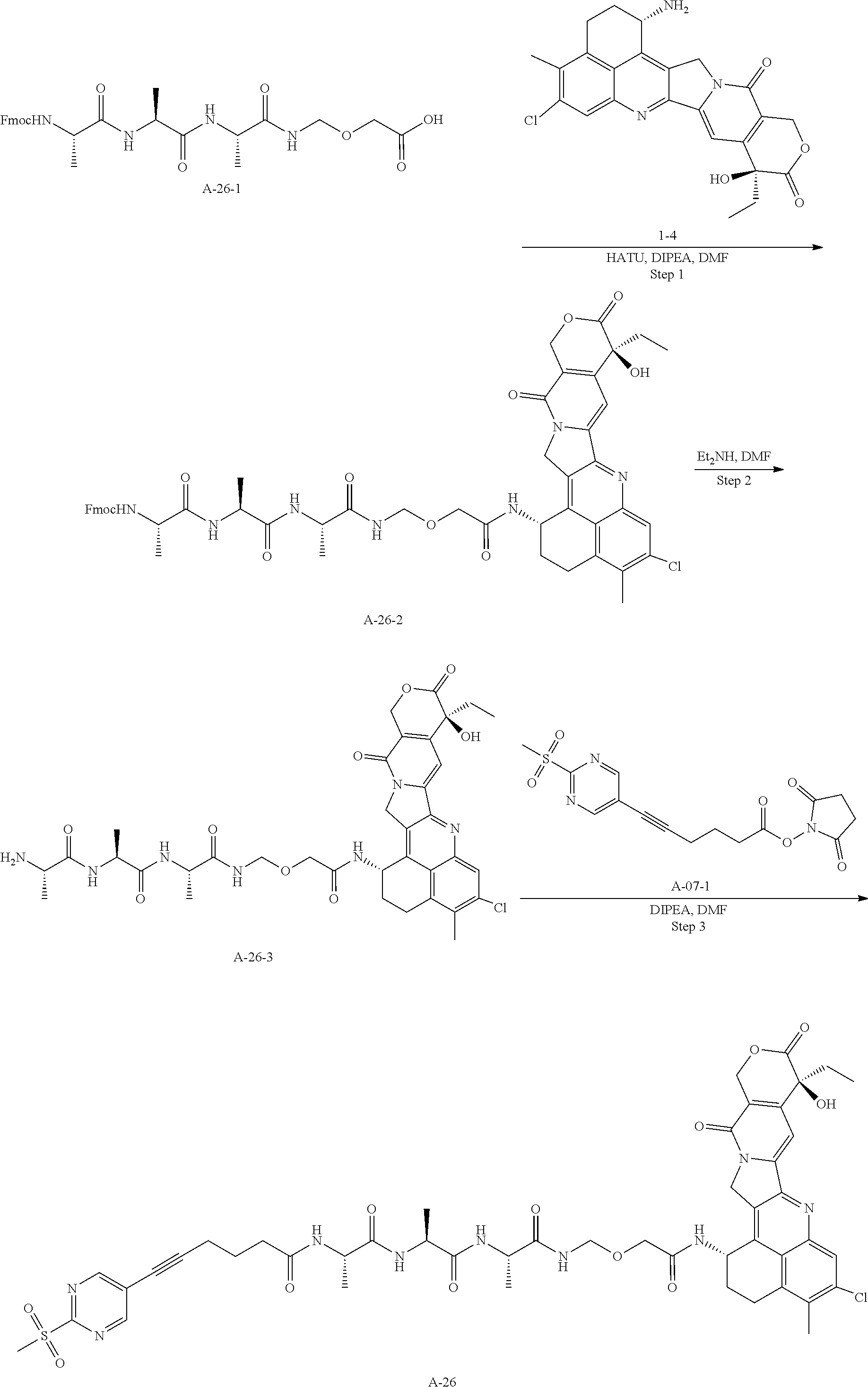

Step 1: Compound A-26-1 (657 mg, 1.22 mmol) and compound 1-4 (500 mg, 1.11 mmol) were dissolved in N,N-dimethylformamide (10 mL), added with HATU (630.67 mg, 1.66 mmol) and N,N-diisopropylethylamine (428 mg, 3.32 mmol), the reaction was carried out under stirring at room temperature for 1 hour. After the reaction was completed, the reaction solution was directly purified by preparative high performance liquid chromatography, and then lyophilized to obtain 700 mg of A-26-2 compound.

The preparation method was as follows:

Chromatographic column: Waters SunFire Prep
C18 OBD (5 μm*19 mm*150 mm)
Mobile phase A: acetonitrile; Mobile phase
B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 18.00 | 90 | 10 | 28 |

Step 2: Compound A-26-2 (500 mg, 0.513 mmol) was dissolved in N,N-dimethylformamide (2 mL), added with diethylamine (75.05 mg, 1.03 mmol), the reaction was carried out at room temperature for 1 hour. After the reaction was completed, the reaction solution was directly purified by preparative high performance liquid chromatography, and then lyophilized to obtain 307 mg of A-26-3 compound.

The preparation method was as follows:

Chromatographic column: Waters SunFire Prep
C18 OBD (5 μm*19 mm*150 mm)
Mobile phase A: acetonitrile; Mobile phase
B: water (0.05% formic acid)

| Time | Mobile phase A | Mobile phase B | Flow rate |
|---|---|---|---|
| [min] | [%] | [%] | [mL/min] |
| 0.00 | 20 | 80 | 24 |
| 2.00 | 20 | 80 | 24 |
| 18.00 | 80 | 20 | 24 |

Step 3: Compound A-26-3 (170 mg, 0.226 mmol) and compound A-07-1 (90.83 mg, 0.249 mmol) were dissolved in N,N-dimethylformamide (10 mL), added with N,N-diisopropylethylamine (29.21 mg, 0.226 mmol). The reaction was carried out under stirring at room temperature for 16 hours. The reaction solution was directly purified by preparative high performance liquid chromatography, and then lyophilized to obtain 50.56 mg of A-26 compound.

The structural characterization data was as follows:

MS m/z (ESI): 1002.4 [M+H]⁺.

The isolation and purification method as follows:

Chromatographic column: Waters SunFire Prep
C18 OBD (5 μm*19 mm*150 mm)
Mobile phase A: acetonitrile; Mobile phase
B: water (0.05% formic acid)

| Time | Mobile phase A | Mobile phase B | Flow rate |
|---|---|---|---|
| [min] | [%] | [%] | [mL/min] |
| 0.00 | 30 | 70 | 28 |
| 2.00 | 30 | 70 | 28 |
| 18.00 | 90 | 10 | 28 |

¹H NMR (400 MHz, DMSO) δ 9.11 (s, 2H), 8.68 (t, J=6.4 Hz, 1H), 8.49 (d, J=8.8 Hz, 1H), 8.16 (s, 1H), 8.10 (d, J=7.2 Hz, 1H), 8.01 (d, J=7.2 Hz, 1H), 7.91 (d, J=6.8 Hz, 1H), 7.31 (s, 1H), 6.55 (s, 1H), 5.65-5.55 (m, 1H), 5.43 (s, 2H), 5.21 (s, 2H), 4.67-4.55 (m, 2H), 4.29-4.15 (m, 3H), 3.98 (s, 2H), 3.41 (s, 3H), 3.25-3.15 (m, 2H), 2.57-2.56 (m, 2H), 2.35-2.27 (m, 2H), 2.22-2.12 (m, 2H), 1.91-1.75 (m, 4H), 1.23-1.09 (m, 9H), 0.87 (t, J=7.2 Hz, 3H).

### Example 11 4-((S)-2-(4-aminobutyl)-35-(4-((6-(2-(methylsulfonyl)pyrimidin-5-yl)hex-5-ynamido)methyl)-1H-1,2,3-triazol-1-yl)-4,8-dioxo-6,12,15,18,24,27,30,33-nonyloxy-3,9-diazapentaazatriamide)benzyl((1S,9R)-5-chloro-9-ethyl-1-(2-hydroxyacetamido)-4-methyl-10,13-dioxo-2,3,9,10,13,15-hexahydro-1H,12H-benzo[d]pyrano[3′,4′:6,7]indolizino[1,2-b]quinolin-9-yl)Carbonate (B-04)

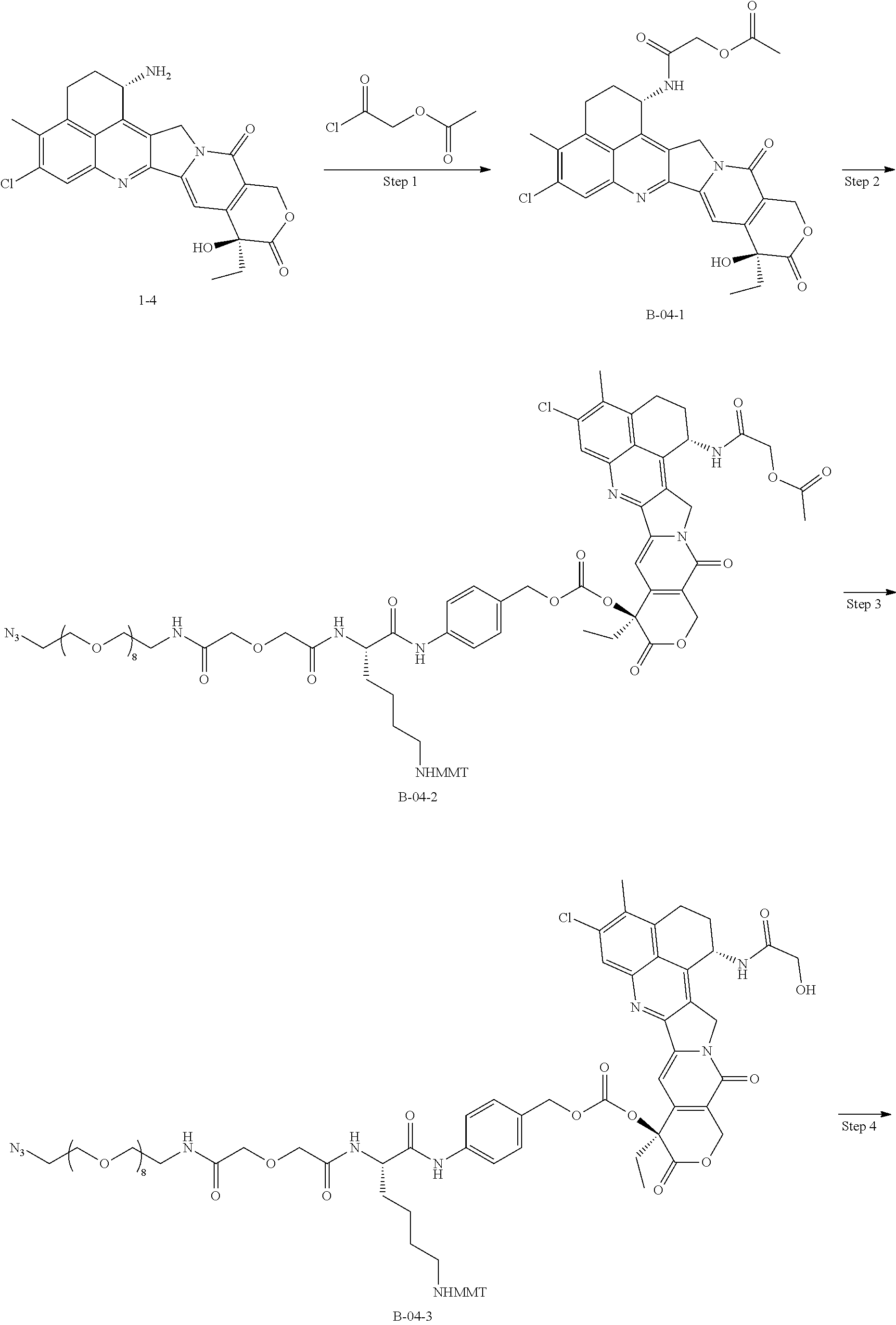

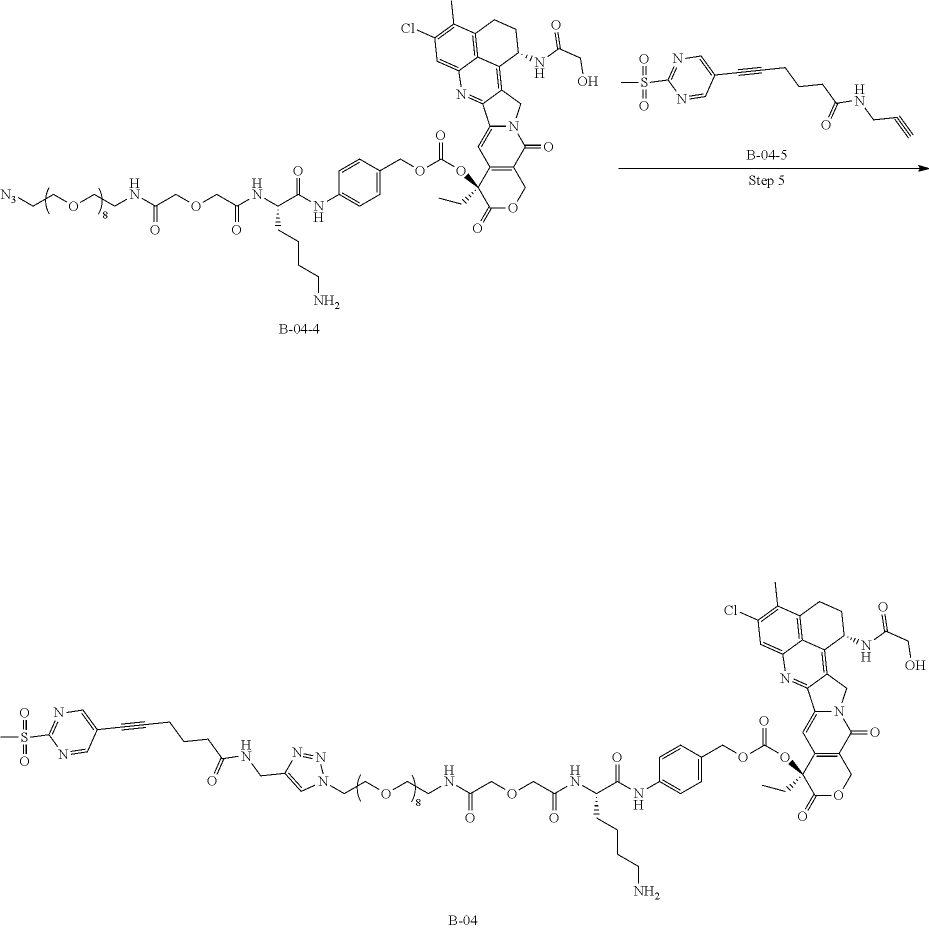

### Step 1: preparation of 2-(((1S,9S)-5-chloro-9-ethyl-9-hydroxyl-4-methyl-10,13-dioxo-2,3,9,10,13,15-hexahydro-1H,12H-benzo[d]pyrano[3′,4′:6,7]indolizino[1,2-b]quinolin-1-yl)amino)-2-oxoethyl acetate (B-04-1)

(1S,9S)-1-amino-5-chloro-9-ethyl-9-hydroxy-4-methyl-1,2,3,9,12,15-hexahydro-10H,13H-benzo[d]pyrano[3′,4′:6,7]indolizino[1,2-b]quinoline-10,13-dione (2 g, 3.65 mmol) was dissolved in DMF (50 mL); DIPEA (1.18 g, 9.12 mmol, 1.59 mL) was added dropwise; acetoxyacetyl chloride (548.12 mg, 4.01 mmol, 431.59 μL) was added dropwise with stirring in an ice bath; and the stirring reaction was continued for 1 hour. The reaction solution was added to 0.1M dilute hydrochloric acid aqueous solution to precipitate a solid, which was filtered. The filter cake was dissolved in dichloromethane and methanol, dried over anhydrous sodium sulfate, filtered, and concentrated to obtain the crude product, purified by silica gel column (methanol/dichloromethane=0%~5%) and concentrated again to obtain the title compound (1.7 g, 3.077 mmol).

The structural characterization data were as follows:

ESI-MS (m/z): 552.2 [M+1]⁺.

### Step 2: Preparation of 2-(((1S,9S)-9-(((4-((S)-35-azido-2-(4-(4-methoxyphenyl)diphenylmethyl)amino)butyl)-4,8-dioxo-6,12,15,18,21,24,27,30,33-nonyloxy-3,9-diazapentaazatriamide)benzyl)oxy)carbonyl)oxy-5-chloro-9-ethyl-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl acetate (B-04-2)

2-(((1S,9S)-5-chloro-9-ethyl-9-hydroxy-4-methyl-10,13-dioxo-2,3,9,10,13,15-hexahydrogen-1H,12H-benzo[d]pyrano[3′,4′:6,7]indeno[1,2-b]quinolin-1-yl)amino)-2-oxoethyl acetate (500 mg, 0.905 mmol) and DMAP (885.33 mg, 7.25 mmol) was dissolved in dry dichloromethane (5 mL), cooled to 0 °C under nitrogen protection, and triphosgene (268.81 mg, 0.905 mmol) dichloromethane solution (5 mL) was added dropwise. keep stirring and reacting for 0.5 hour. Slowly added in(S)-2-(32-azido-5-oxo-3,9,12,15,18,21,24,27,30-nonyloxy-6-azatrinitroamino)-N-(4-(hydroxymethyl)phenyl)-6-(((4-methoxyphenyl)diphenylmethyl)amino)hexanamide (1.44 g, 1.36 mmol) dichloromethane solution dropwise, naturally returned to room temperature and reacted for 4 hours. The reaction solution was quenched with water, extracted with dichloromethane 3 times (100 ml×3), combined the organic phases, washed with saturated brine, dried, and concentrated, and purified by silica gel column (MeOH/DCM=0%~5%) to obtain the title compound (498 mg, 0.304 mmol).

The structural characterization data were as follows:

ESI-MS (m/z): 1352.8 [M+1]⁺.

### Step 3: preparation of 4-((S)-35-azido-2-(4-((4-methoxyphenyl)diphenylmethyl)amino)butyl)-4,8-dioxo-6,12,15,18,24,27,30,33-nonyloxy-3,9-diazapentaazatriamide)benzyl)((1S,9S)-5-chloro-9-ethyl-1-(2-hydroxyacetamido)-4-methyl-10,13-dioxo-2,3,9,10,13,15-hexahydro-1H, 12H-benzo[de]pyrano[3′,4′:6,7]indolizino[1,2-b]quinolin-9-yl)carbonate (B-04-3)

2-(((1S,9S)-9-(((4-((S)-35-azido-2-(4-(4-methoxyphenyl)diphenylmethyl)amino)butyl)-4,8-dioxo-6,12,15,18,21,24,27,30,33-nonyloxy-3,9-diazapentaazatriamide)benzyl)oxy)carbonyl)oxy-5-chloro-9-ethyl-4-methyl-10,13-dioxo-2,3,9,10,13,15-hexahydro-1H,12H-benzo[de]pyrano[3′,4′:6,7]indolizino[1,2-b]quinolin-1-yl)amino)-2-oxoethyl acetate (200 mg, 0.122 mmol) was dissolved in THF (3 mL) and MeOH (3 mL), sodium carbonates (25.88 mg, 0.224 mmol) aqueous solution (1 mL) was added dropwise with stirring, and the stirring reaction was continued for 1 hour. Neutralizing the reaction solution by adding dilute hydrochloric acid solution dropwise, concentrating under reduced pressure, then directly went to the next step.

The structural characterization data were as follows:

ESI-MS (m/z): 1596.7 [M+1]⁺.

### Step 4: Preparation of 4-((S)-2-(4-aminobutyl)-35-azido-4,8-dioxo-6,12,15,18,21,24,27,30,33-nonyloxy-3,9-diazapentaazatriamide)benzyl)((1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (B-04-4)

4-((S)-35-azido-2-(4-((4-methoxyphenyl)diphenylmethyl)amino)butyl)-4,8-dioxo-6,12,15,18,24,27,30,33-nonyloxy-3,9-diazapentaazatriamide)benzyl)((1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (190 mg, 119.04 μmol) was dissolved in dichloromethane (5 mL), trifluoroacetate (0.5 mL) was added, then continued the reaction for 1 hour. Saturated sodium bicarbonate aqueous solution was added dropwise into the reaction solution for neutralization, then separated the liquids, and concentrated the organic phase to obtain the crude product. The title compound (95 mg, 69.35 μmol) was obtained after purification by reverse-phase chromatography column (acetonitrile/1% aqueous formic acid=0%~50%) and freeze-drying.

The structural characterization data were as follows:

ESI-MS (m/z): 1323.6 [M+1]⁺.

### Step 5: Preparation of 4-((S)-2-(4-aminobutyl)-35-(4-((6-(2-(methylsulfonyl)pyrimidin-5-yl)hex-5-ynamido)methyl)-1H-1,2,3-triazol-1-yl)-4,8-dioxo-6,12,15,18,24,27,30,33-nonyloxy-3,9-diazapentaazatriamide)benzyl((1S,9R)-5-chloro-9-ethyl-1-(2-hydroxyacetamido)-4-methyl-10,13-dioxo-2,3,9,10,13,15-hexahydro-1H,12H-benzo[d]pyrano[3′,4′:6,7]indolizino[1,2-b]quinolin-9-yl)carbonate (B-04)

4-((S)-2-(4-aminobutyl)-35-azido-4,8-dioxo-6,12,15,18,21,24,27,30,33-nonyloxy-3,9-diazapentaazatriamide)benzyl)((1S,9S)-5-chloro-9-ethyl-1-(2-hydroxyacetamido)-4-methyl-10,13-dioxo-2,3,9,10,13,15-hexahydro-1H,12H-benzo[de]pyrano[3′,4′:6,7]indolizino[1,2-b]quinolin-9-yl)carbonate (90 mg, 0.066 mmol) and 6-(2-(methylsulfonyl)pyrimidin-5-yl)-N-(prop-2-yn-1-yl)hex-5-ynamide (24.07 mg, 0.079 mmol) were dissolved in DMSO (2 mL) and water (0.2 mL), cuprous bromide (9.42 mg, 0.066 mmol) was added, then kept string for 2 hours. The reaction solution was directly filtered and the crude product was concentrated, which was purified by preparative high performance liquid chromatography and lyophilized to obtain the title compound (42.2 mg, 24.69 μmol).

The structural characterization data were as follows: ESI-MS (m/z): 1628.7 [M+1]⁺.

The method of preparative high performance liquid chromatography as follows:

Chromatographic column: SunFire Prep C18
OBD 19 mm × 150 mm × 5.0 μm
Mobile phase A: acetonitrile; Mobile
phase B: water (0.05% formic acid)

| [min] | Mobile phase A | Mobile phase B | Flow rate |
|---|---|---|---|
| Time | [%] | [%] | [mL/min] |
| 0.00 | 10 | 90 | 28 |
| 2.00 | 10 | 90 | 28 |
| 18.00 | 90 | 10 | 28 |

### Example 12 Preparation of Antibodies

Human PTK7 protein was adopted to immunize wild-type mice; the serum titer was monitored by ELISA and cell flow cytometry; according to the titer results, the optimal mouse was selected, from which the spleen cells were taken for fusion, screening and subcloning; the activity of different monoclones for binding to human\monkey proteins, cells, endocytosis and other activities were detected; the clones were selected for humanization; and finally a 64A10 humanized antibody variable regions (a heavy chain variable region, SEQ ID NO:7; a light chain variable region, SEQ ID NO:8), and a 101A6 humanized antibody variable regions (heavy chain variable region, SEQ ID NO:3; light chain variable region, SEQ ID NO:4) were obtained; the above heavy chain variable region sequences were fused to human IgG1 heavy chain constant region (SEQ ID NO:43) respectively, and the above light chain variable region sequences were fused to a human k light chain constant region (SEQ ID NO:44) respectively so as to form two complete humanized antibodies (see Table 1); the Nanjing GenScript Biotech Co., Ltd. was entrusted to perform codon optimization and gene synthesis on the antibodies, and construct the antibodies into the pTT5 plasmid; and the heavy chain and light chain plasmids were transfected into CHO-S cells at the same time, and Protein A was used for purifying the expressed antibody in the supernatant to obtain the corresponding antibodies 64A10HZ and 101A6HZ. Meanwhile, in order to reduce the non-target toxicity mediated by antibody Fc receptors, a humanized 101A6 heavy chain variable region was fused to a mutated human IgG1 heavy chain constant region (SEQ ID NO: 45), and the expressed antibody was named as 101A6HZm. The hIgG1 is an anti-chicken lysozyme antibody fused to a human IgG1 heavy chain constant region, and the hIgG1m is an anti-chicken lysozyme antibody fused to a mutated human IgG1 heavy chain constant region.

The PTK7 control antibody was obtained from Hu24 in Patent CN201580030255. After codon optimization, the antibody heavy and light chain nucleotide sequences were synthesized and cloned into a pTT5 vector, respectively, and expressed and purified according to the above method.

TABLE 1
Sequence information of 64A10HZ and 101A6HZ (SEQ ID NO: )

| | | | Definition | | | | | | | | |
|---|---|---|---|---|---|---|---|---|---|---|---|
| Antibody | VH | VL | mode | CDR H1 | CDR H2 | CDR H3 | CDR L1 | CDR L2 | CDR L3 | CH | CL |
| 101A6 | 1 | 2 | Chothia | 11 | 12 | 13 | 14 | 15 | 16 | 43 | 44 |
| | | | Kabat | 17 | 19 | 13 | 14 | 15 | 16 | | |
| | | | IMGT | 20 | 21 | 22 | 23 | 24 | 16 | | |
| | | | AbM | 25 | 26 | 13 | 14 | 15 | 16 | | |
| 101A6HZ | 3 | 4 | Chothia | 11 | 12 | 13 | 14 | 15 | 16 | 43 | 44 |
| | | | Kabat | 17 | 18 | 13 | 14 | 15 | 16 | | |
| | | | IMGT | 20 | 21 | 22 | 23 | 24 | 16 | | |
| | | | AbM | 25 | 26 | 13 | 14 | 15 | 16 | | |
| 101A6HZm | 3 | 4 | Chothia | 11 | 12 | 13 | 14 | 15 | 16 | 45 | 44 |
| | | | Kabat | 17 | 18 | 13 | 14 | 15 | 16 | | |
| | | | IMGT | 20 | 21 | 22 | 23 | 24 | 16 | | |
| | | | AbM | 25 | 26 | 13 | 14 | 15 | 16 | | |
| 64A10 | 5 | 6 | Chothia | 27 | 28 | 29 | 30 | 31 | 32 | 43 | 44 |
| | | | Kabat | 33 | 35 | 29 | 30 | 31 | 32 | | |
| | | | IMGT | 36 | 37 | 38 | 39 | 40 | 32 | | |
| | | | AbM | 41 | 42 | 29 | 30 | 31 | 32 | | |
| 64A10HZ | 7 | 8 | Chothia | 27 | 28 | 29 | 30 | 31 | 32 | 43 | 44 |
| | | | Kabat | 33 | 34 | 29 | 30 | 31 | 32 | | |
| | | | IMGT | 36 | 37 | 38 | 39 | 40 | 32 | | |
| | | | AbM | 41 | 42 | 29 | 30 | 31 | 32 | | |
| 64A10HZ05 | 9 | 10 | Chothia | 27 | 28 | 29 | 30 | 31 | 32 | 43 | 44 |
| | | | Kabat | 33 | 34 | 29 | 30 | 31 | 32 | | |
| | | | IMGT | 36 | 37 | 38 | 39 | 40 | 32 | | |
| | | | AbM | 41 | 42 | 29 | 30 | 31 | 32 | | |

### Example 13 Antibody Conjugation and Detection

### Conjugation of Antibody and C-01

The 64A10HZ, 101A6HZ, Hu24 or hIgG1m antibodies were taken and diluted with 20 mM PB+0.1 M EDTA (pH 7.60); then the pH was adjusted to 7.60 with 1 M of Na₂HPO₄ solution; and 10 mM of TCEP (tris(2-carboxyethyl)phosphine, pH 7.60) solution was added, uniformly mixed and allowed to stand at room temperature for 1.5 h. The solution of C-01 (10 mM, 5 eq. of antibody) in DMSO was added to the above mixture, and then the resultant mixture was uniformly mixed and allowed to stand at room temperature for 2 h; then NAP-5 gel column (Cytiva) was adopted to replace the buffer solution into 20 mM of a histidine buffer solution with pH of 6.0 so as to obtain the antibody-drug conjugate. The DAR value determined by mass spectrometry is shown in Table 2.

### Conjugation of 101A6HZ and A-05

1.938 mol of 101A6HZ antibody (25.8 mg/mL) was taken and diluted with 96.9 μL of 20 mM PB+0.1 M EDTA (pH 7.60); then the pH was adjusted to 7.60 with 1 M of Na₂HPO₄ solution; and 10 mM of TCEP (tris(2-carboxyethyl)phosphine, 83.1 μL, pH 7.60) solution was added, uniformly mixed and allowed to stand at room temperature for 1.5 h. The solution of A-05 (10 mM, 5 eq. of antibody) in DMSO was added to the above mixture, and then the resultant mixture was uniformly mixed and allowed to stand at room temperature for 2 h; then NAP-5 gel column (Cytiva) was adopted to replace the buffer solution by 20 mM of a histidine buffer solution with pH of 6.0 so as to obtain the antibody-drug conjugate (i.e., ADC 101A6HZ-A-05-4). The DAR value determined by mass spectrometry was 3.73.

### Conjugation of Antibodies and A-05, B-01, A-26, B-04

The 101A6HZ, 101A6HZm, 64A10HZ, Hu24, hIgG1 or hIgG1m antibodies were taken and diluted with 20 mM PB+0.1 M EDTA (pH 7.60); then the pH was adjusted to 7.60 with 1 M of Na₂HPO₄ solution; and 10 mM of TCEP (tris(2-carboxyethyl)phosphine, pH 7.60) solution was added, uniformly mixed and allowed to stand at room temperature for 1.5 h. The solution of A-05, B-01, A-26, or B-04 (10 mM, 10 eq. of antibody) in DMSO was added to the above mixture, and then the resultant mixture was uniformly mixed and allowed to stand at room temperature for 2 h; and then NAP-5 gel column (Cytiva) was adopted to replace the buffer solution by 20 mM of a histidine buffer solution with pH of 6.0 so as to obtain the antibody-drug conjugate.

After the conjugation reaction, the methylsulfonyl group of A-05, B-01, A-26, or B-04 is displaced, and the cysteine sulfhydryl groups in the referenced antibody are directly bonded to the pyrimidine ring as shown in ADC A-05, ADC B-01, ADC A-26, or ADC B-04.

The DAR value determined by mass spectrometry is shown in Table 2.

The drug/antibody ratio (DAR) of the conjugated sample was determined as follows:

LC-MS molecular weight analysis was performed on the conjugated ADC samples.

Chromatographic determination conditions:
Liquid chromatography column: Thermo MAbPac RP 3.0*100 mm;
Mobile phase A: 0.1% FA/H₂O; mobile phase B: 0.1% FA/ACN;
Flow rate: 0.25 ml/min; sample chamber temperature: 8 °C; column
temperature: 60 °C; Injection volume: 2 μl;

| Time (min) | 2 | 20 | 22 | 25 | 26 | 30 |
|---|---|---|---|---|---|---|
| Mobile phase A (volume %) | 75 | 60 | 5 | 5 | 75 | 75 |
| Mobile phase B (volume %) | 25 | 40 | 95 | 95 | 25 | 25 |

Mass spectrometry determination conditions:

Mass spectrometry model: AB Sciex Triple TOF 5600+;

GS1 35; GS2 35; CUR 30; TEM 350; ISVF 5500; DP 200; CE 10; Accumulation time 0.5 s;

m/z 600-4000; Time bins to sum 40.

TABLE 2
ADC quality test results

| Drug-linker No. | ADC No. | Purity SEC % | DAR |
|---|---|---|---|
| A-05 | 64A10HZ-A-05 | 95.9 | 8.00 |
| A-05 | 101A6HZ-A-05 | 98.8 | 8.03 |
| B-01 | 64A10HZ-B-01 | 92.7 | 8.01 |
| B-01 | 101A6HZ-B-01 | 99.4 | 7.97 |
| C-01 | 64A10HZ-C-01 | 90.0 | 3.4 |
| C-01 | 101A6HZ-C-01 | 89.1 | 3.96 |
| C-01 | Hu24-C-01 | 92.5 | 4.08 |
| A-05 | 101A6HZ-A-05-4 | 99.2 | 3.73 |
| A-05 | 101A6HZm-A-05 | 97.6 | 8.05 |
| A-05 | hIgG1-A-05 | 98.5 | 8.02 |
| A-05 | hIgG1m-A-05 | 97.9 | 7.18 |
| C-01 | hIgG1m-C-01 | 98.1 | 2.73 |
| A-26 | 101A6HZm-A-26 | 96.7 | 8.15 |
| B-04 | 101A6HZm-B-04 | 93.8 | 8.47 |

### Example 14 Detection of Activity of Antibody-Drug Conjugate

### 1. In Vivo Efficacy Detection of Different Antibody-Drug Conjugate in NCI-H358 Model

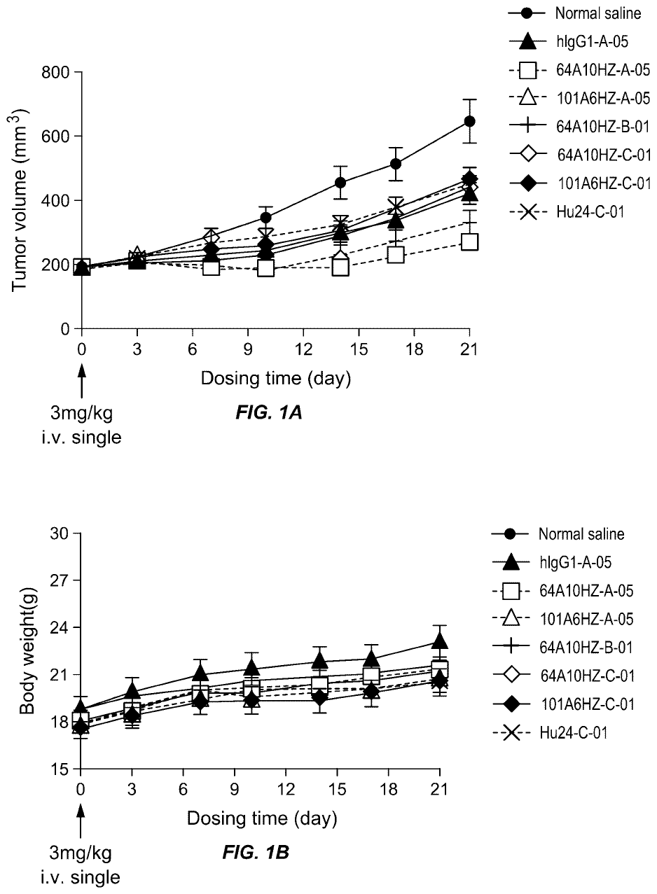

Human non-small cell lung cancer NCI-H358 cells (NANJING COBIOER BIOSCIENCES CO., LTD) were cultured in vitro in a single layer, the culture conditions was: 10% of fetal bovine serum being added in a RPMI1640 medium, and incubation was carried out in an incubator containing 5% CO₂ at 37 °C 5×10⁶ NCI-H358 cells were inoculated subcutaneously in the right underarm of each mouse, and suspended in 0.05 ml PBS+0.05 ml Matrigel. When the average tumor volume grew to 150-200 mm³, the mice with irregular tumor volume and too small or too large tumor volume were excluded, and the remaining mice were randomly divided into 8 groups according to tumor volume and animal weight, with 6 mice in each group. The drug was given IV in single dose (3 mg/kg); after dosing, the tumor was measured twice a week with a vernier caliper, and the tumor volume was calculated according to the following formula: V=0.5a×b², wherein a and b respectively represents the long diameter and short diameter of the tumor; the anti-tumor drug efficacy was evaluated through tumor growth inhibition rate TGI (%), and calculation formula was: TGI (%) (tumor volume)=[1-(T_{Vt}−T_{V0})/(C_{Vt}−C_{V0})]×100%; when the tumor was in remission, TGI (%) (tumor volume)=100%−(T_{Vt}-T_{V0})/T_{V0}×100%. T_{V0} was the average tumor volume of the test drug group at the time of group dosing; T_{Vt} was the average tumor volume of the test drug group at day t after dosing; C_{V0} was the average tumor volume of the vehicle group (normal saline) at the time of group dosing; and C_{Vt} was the average tumor volume of the vehicle group on day t after dosing. If the tumor shrunk compared with the initial volume, that is, Vₜ<V₀, it was defined as a tumor partial response (PR); and if the tumor completely disappeared, it was defined as a tumor complete response (CR). Animal death was observed and recorded every day, and the specific results are shown in Table 3, and FIGS. 1A-1B.

TABLE 3
Analysis of efficacy of different antibody-drug conjugates on human non-small cell
lung cancer cells in NCI-H358 subcutaneous tumor-bearing mouse model (N = 6)

| Drug | V_{P0}(mm³, mean ± SEM) | V_{P21}(mm³, mean ± SEM) | TGI (%) | P Value |
|---|---|---|---|---|
| Normal saline | 191.91 ± 11.32 | 647.23 ± 67.63 | / | / |
| hIgG1-A-05 | 190.46 ± 9.11 | 421.66 ± 34.42 | 49.22* | 0.01398 |
| 64A10HZ-A-05 | 191.59 ± 9.35 | 268.77 ± 26.92 | 83.05*** | 0.00040 |
| 101A6HZ-A-05 | 190.85 ± 9.52 | 281.42 ± 24.19 | 80.11*** | 0.00047 |
| 64A10HZ-B-01 | 190.68 ± 7.73 | 331.38 ± 36.46 | 69.10** | 0.00211 |
| 64A10HZ-C-01 | 188.74 ± 9.49 | 443.05 ± 33.63 | 44.15* | 0.02219 |
| 101A6HZ-C-01 | 192.06 ± 9.41 | 465.40 ± 40.01 | 39.97* | 0.04320 |
| Hu24-C-01 | 189.77 ± 6.92 | 449.71 ± 50.28 | 42.91* | 0.04107 |

Note:
T—test,
*P < 0.05,
**P < 0.01,
***P < 0.001 means significant difference compared with normal saline group.

The results show that: single intravenous dosing was performed, different conjugated drugs had significant drug effects on human non-small cell lung cancer cells in NCI-H358 mouse subcutaneous xenograft tumor animal model, TGI (%): A-05 conjugate was better than C-01 conjugates, and animals in each group tolerated better.

### 2. In Vivo Efficacy Detection of Different Antibody Drug Conjugates in A431 Model

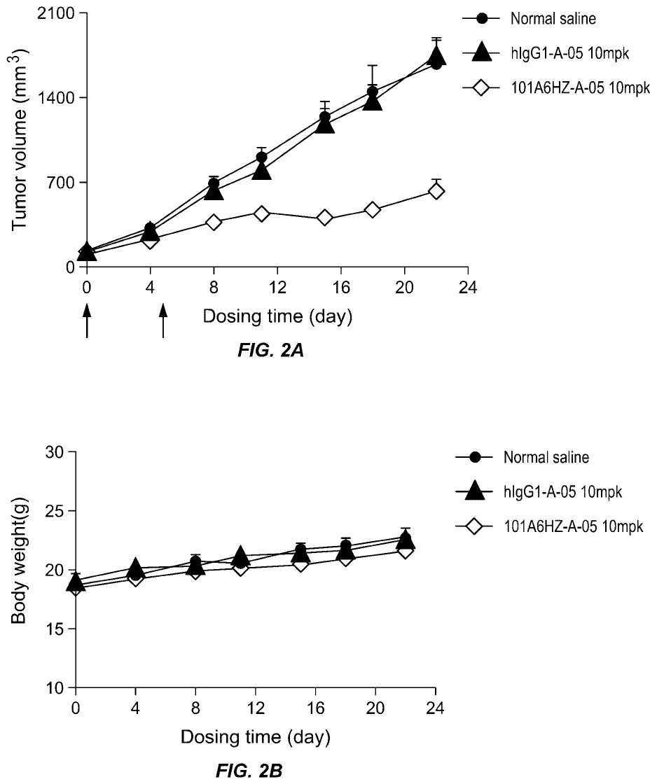

Human epidermal squamous cell carcinoma A431 cells (NANJING COBIOER BIOSCIENCES CO., LTD) were cultured in conditions as follows: 10% of fetal bovine serum was added into a DMEM medium, and incubation was carried out in an incubator containing air having 5% CO₂ at 37 °C 5×10⁶ A431 cells were inoculated subcutaneously in the right underarm of each mouse, and suspended in 0.1 ml PBS. When the average tumor volume grew to about 100-150 mm³, the mice with too small or too large tumor volume were excluded, and the remaining mice were randomly divided into 3 groups according to the tumor volume and animal weight, with 5 mice in each group; the drug was given IV once every week, twice in total. The tumor volume and body weight were measured twice a week after dosing, and the specific results are shown in Table 4, and FIGS. 2A-2B.

TABLE 4
Analysis of efficacy of antibody-drug conjugates on
human epidermal squamous cell carcinoma cells in A431
subcutaneous tumor-bearing mouse model (N = 5)

| | Drug | V_{P0}(mm³, | V_{P22}(mm³, | | |
|---|---|---|---|---|---|
| | administration | mean ± | mean ± | TGI | |
| Drug | dosage (mg/kg) | SEM) | SEM) | (%) | P Value |
| Normal | / | 128.36 ± | 1675.96 ± | / | / |
| saline | | 9.62 | 199.83 | | |
| hIgG1- | 10 | 126.58 ± | 1749.78 ± | −4.89 | 0.77291 |
| A-05 | | 9.30 | 145.64 | | |
| 101A6HZ- | 10 | 127.77 ± | 624.86 ± | 67.88** | 0.00148 |
| A-05 | | 9.58 | 96.98 | | |

Note:
T—test,
**P < 0.01 means significant difference compared with normal saline group.

The results show that: 101A6HZ-A-05 had significant efficacy under the condition of 10 mg/kg.

### 3. Detection of Binding of Anti-Human PTK7 Antibody and Conjugate Thereof to PTK7 Overexpressed Cells of Different Genera and Species

The capability of the antibody and the conjugate thereof to bind to CHO-PTK7 cells was monitored by flow cytometer (Beckman, Model: Cytoflex). The CHO-PTK7 cells cultured in suspension (human: hPTK7, monkey: cPKT7, mouse: mPTK7, rat: rPTK7) were counted; the cells were collected at a density of 2×10⁵ cells/well, washed twice with 1×PBS, and resuspended in 1% BSA solution; then the cells were transferred to a 96-well deep plate, 50 μl per well; the antibody and the drug conjugate thereof were diluted with 1% BSA, respectively, starting from 10 μg/ml, and diluting was carried out in a triple gradient manner; then 50 μl of the diluted antibody or antibody-drug conjugate were added to the deep plate containing cells, and incubated at 4 °C for 40 min; the cells were washed twice with PBS, then 50 μl of diluted secondary antibody was added to each well, and uniformly mixed, and the cells were incubated at 4 °C for 30 min; and the cells were washed with PBS twice, then the cells were resuspended in 400 μl of PBS, and detected by flow cytometry. Data processing: the median fluorescence signal value was imported into GraphPad Prism 6 software to calculate EC₅₀, and the results are shown in Table 5. The results show that the antibody 101A6HZ and 101A6HZm conjugates according to the present invention could bind to overexpressed human and monkey PTK7 cells, but not to mouse and rat PTK7 cells.

TABLE 5
Affinity determination result of anti-human PTK7_ADC cells

| | EC₅₀(ng/ml) | | | |
|---|---|---|---|---|
| Drug | hPTK7 | cPTK7 | mPTK7 | rPTK7 |
| 101A6HZm-A-05 | 418.70 | 551.20 | Not bound | Not bound |
| 101A6HZ-C-01 | 147.50 | 114.70 | Not bound | Not bound |

### 4. Detection of Cell Affinity of Anti-Human PTK7 Antibody and Conjugate Thereof

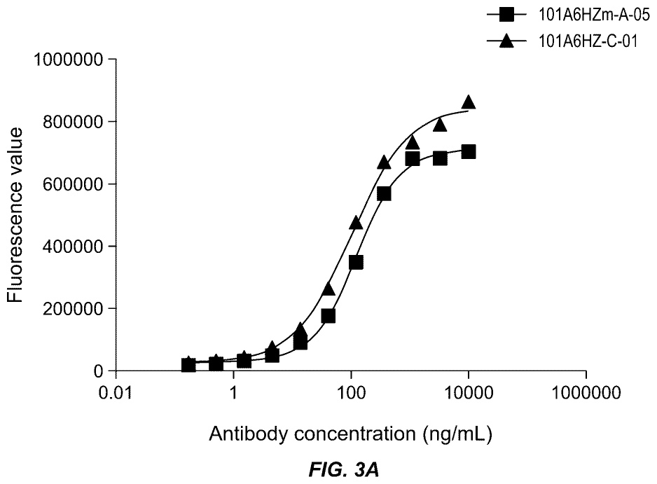

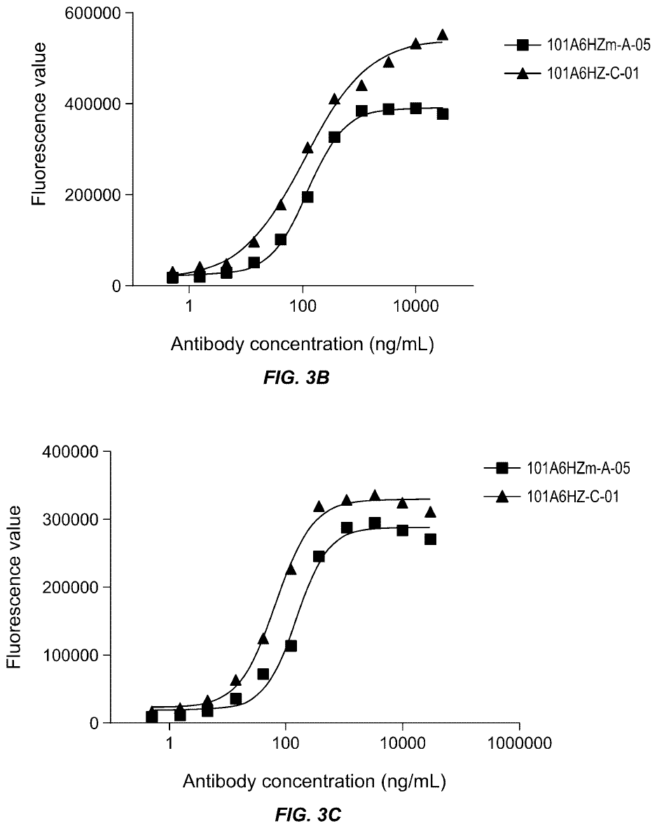

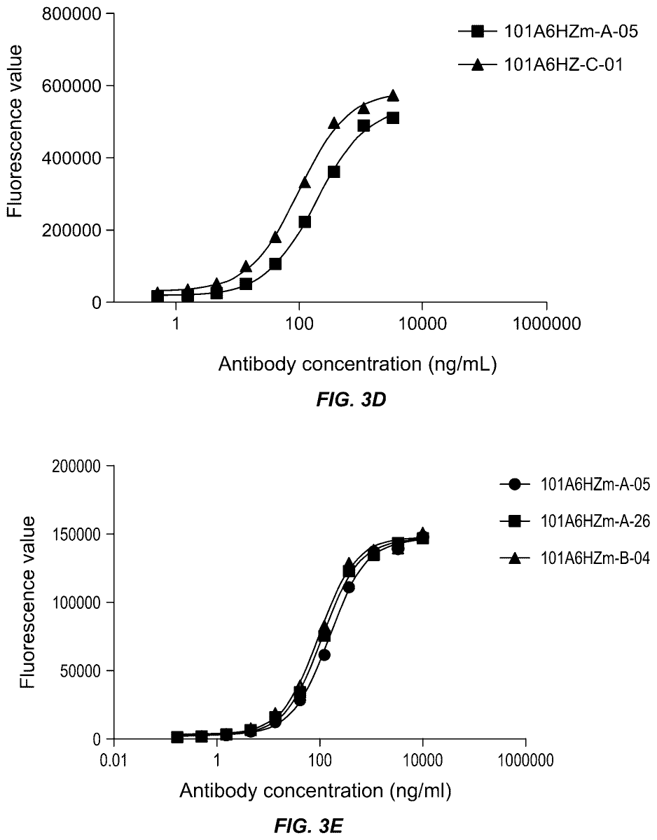

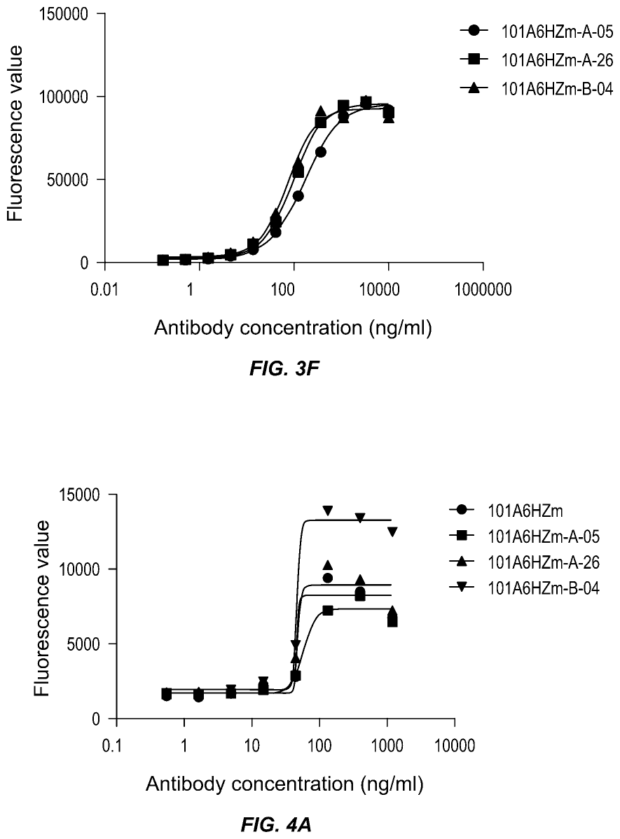

The affinity of the anti-human PTK7 antibody and the conjugated ADC to OVCAR3 (ATCC), NCI-H358, HCC1806 (ATCC), A431, NCI-H520 (Nanjing Cobioer Biosciences Co., LTD.), DU4475 cells was detected by flow cytometer (Beckman, Model: Cytoflex). The adherent cells OVCAR3, NCI-H358, HCC1806, A431, NCI-H520 were digested by trypsin, the suspension cells DU4475 was mixed with pipetting, an appropriate amount of cells were counted and collected, and the detection steps of flow cytometry were the same as above. The results are shown in Table 6-1, 6-2 and FIGS. 3A-3F. The results show that after the antibodies 101A6HZ and 101A6HZm according to the present invention were conjugated into ADC, they could still bind tumor cells with high affinity.

TABLE 6-1
Affinity determination result of anti-human PTK7 ADC to cells

| | EC₅₀(ng/ml) | | | |
|---|---|---|---|---|
| Drug | OVCAR3 | NCI-H358 | HCC1806 | A431 |
| 101A6HZm-A-05 | 127.20 | 123.60 | 152.0 | 154.10 |
| 101A6HZ-C-01 | 102.60 | 108.10 | 67.07 | 97.40 |

TABLE 6-2
Results of anti-human PTK7 ADC cell affinity detection

EC₅₀(ng/ml)

| Drug | NCI-H520 | DU4475 |
|---|---|---|
| 101A6HZm-A-05 | 157.0 | 173.0 |
| 101A6HZm-A-26 | 114.3 | 89.13 |
| 101A6HZm-B-04 | 97.29 | 74.81 |

### 5. Detection of Cell Endocytosis of Anti-Human PTK7 Antibody and Conjugate Thereof

### 5.1 Endocytosis Detection Method I

HCC1806 and OVCAR3 cells were digested with trypsin, resuspended and then counted; the cells were collected at a density of about 2×10⁵ cells per well; the cells were suspended with 1% of BSA in 50 μl volume per well, spread on the deep plate; the antibody and corresponding ADC were added and incubated at 4 °C for 60 min; cells were washed twice with 1% BSA, then a second antibody (Anti-human IgG Alexa Fluor 488) was added, and incubated at 4 °C for 30 min; the endocytosis at different temperatures (PLOS ONE 10 (4): e0124708) was detected with reference to the literature; and after incubating, 150 μl of 1% BSA was added to resuspend and test on the machine. Data analysis: the fluorescence signal value after quenching at 37 °C was adopted to make a fitting curve at different concentration points, and the EC₅₀ was calculated. The results are shown in Table 7. The candidate antibody conjugate had good endocytic activity.

TABLE 7
Detection of cell endocytosis of ADC

| | EC₅₀(ng/ml) | | | |
|---|---|---|---|---|
| Drug | OVCAR3 | NCI-H358 | HCC1806 | A431 |
| 101A6HZm-A-05 | 168.70 | 178.40 | 185.0 | 96.62 |
| 101A6HZ-C-01 | 65.29 | 96.20 | 106.3 | 68.02 |

### 5.2 Endocytosis Detection Method II

The adherent cells NCI-H358. HCC1806. OVCAR3. NCI-H520 were digested by trypsin, the suspension cells DU4475 was mixed with pipetting, then the cells were counted and put into a 96-well plate with 10,000 cells in 100 μl medium per well. Then the cells were cultured in 37 °C in the incubator. In the next day, the antibody or ADC samples were diluted with complete culture medium, start from 4.8 μg/ml, 3-fold dilution, gradient dilution in 8 concentrations. The pHrodo-red, which was diluted with complete culture medium to 12 μg/ml, was used for labeling the antibody or ADC samples. 30 μl diluted antibody/ADC samples and 30 μl of diluted pHrodo-red sample were mixed in a 96-well plate, incubated in dark at room temperature for 30 minutes. Then 50 μl medium per well was removed from the cells, 50 μl labelled antibody or ADC was added into cells, the cells were incubated at 37 °C 5% CO₂ in the incubator for 24 hours. On the next day, the culture supernatant was removed, the cells were washed with PBS once, and digested by adding 40 μl 0.25% trypsin per well, then neutralized with 100 μl complete culture medium, mixed well by pipetting up and down. Flow cytometry was used to detect fluorescence signals excited by excitation wavelength at 561 nm.

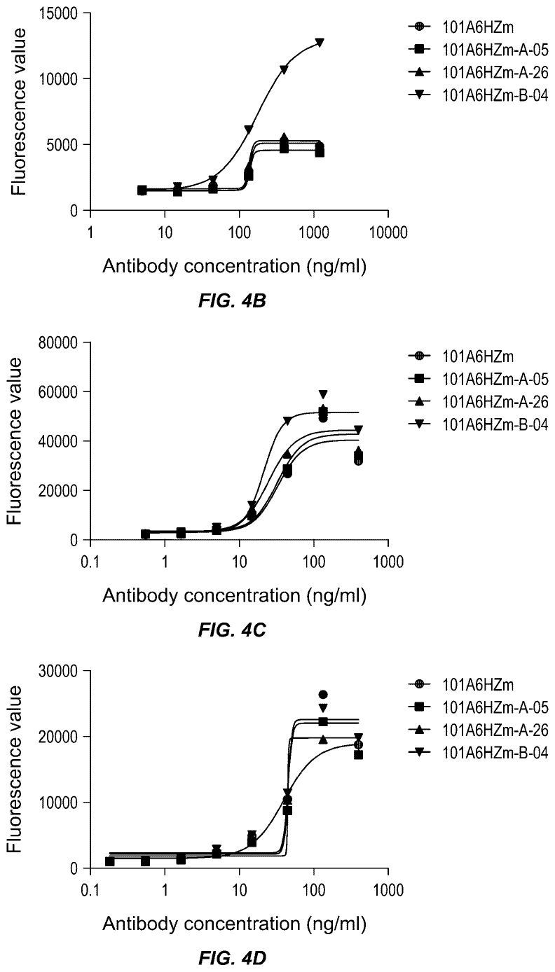

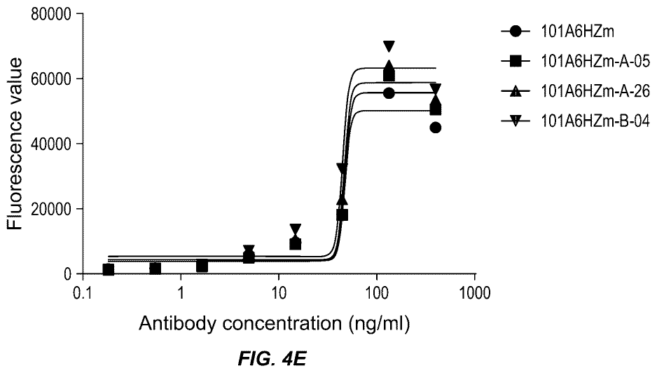

Data procession: the median value was exported and imported into GraphPad Prism 6 software, EC₅₀ was calculated. Results were shown in Table 8 and FIGS. 4A-4E.

TABLE 8
Detection of endocytosis of antibodies
and antibody-drug conjugates

| | EC₅₀(ng/ml) | | | | |
|---|---|---|---|---|---|
| Drug | NCI-H358 | HCC1806 | OVCAR3 | NCI-H520 | DU4475 |
| 101A6HZm | 47.43 | 137.8 | 32.94 | 45.40 | 42.16 |
| 101A6HZm- | 58.64 | 138.0 | 32.53 | 45.78 | 47.50 |
| A-05 | | | | | |
| 101A6HZm- | 47.34 | 134.5 | 24.98 | 38.73 | 46.56 |
| A-26 | | | | | |
| 101A6HZm- | 47.36 | 176.8 | 21.20 | 44.90 | 44.91 |
| B-04 | | | | | |

### 6. In Vitro Cytotoxicity Detection of Anti-Human PTK7 Antibody-Drug Conjugate

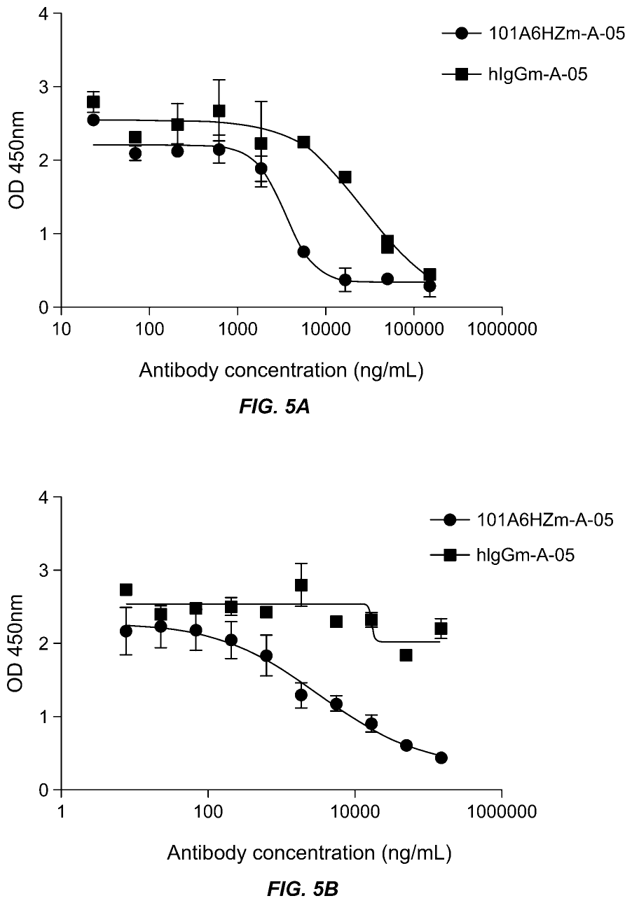

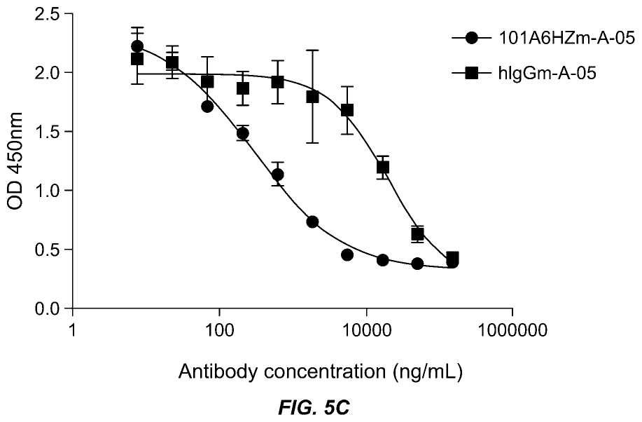

FADU (NANJING COBIOER BIOSCIENCES CO., LTD), HCC1806, DU4475 (NANJING COBIOER BIOSCIENCES CO., LTD) and H358-hPTK7 cells were digested by pancreatin; the optimal number of cells were selected according to the proliferation curve of each tumor cell and plated, and then cultured overnight at 37 °C Each antibody and conjugate were diluted with culture medium, added into to the cells, and continuously cultured 5-6 days. CCK8 was added to the cells, and continuously cultured for 2-4 h; the value at OD450 nm was read with a microplate reader; the killing activity was calculated; and the results are shown in Table 9 and FIGS. 5A-5C. The killing activity of the conjugate was significantly stronger than that of the negative antibody conjugate, indicating that the killing effect was mediated by the target.

TABLE 9
In vitro cell killing results of anti-human PTK7 ADC

| | IC⁵⁰(nM) | | | |
|---|---|---|---|---|
| Drug | FADU | HCC1806 | DU4475 | H358-hPTK7 |
| hIgGm-A-05 | 187.54 | / | 130.17 | / |
| 101A6HZm-A-05 | 22.51 | 21.59 | 1.96 | 1.03 |

Note:
“/” means no killing activity.

### 7. Hydrophilicity Detection of Anti-Human PTK7 Antibody and Conjugate

Agilent 1260 and analytical column TSKgel Butyl-NPR were adopted to detect the hydrophilicity of antibody and conjugate; an appropriate amount of the test sample was taken and diluted with diluent (0.75 mol/L (NH₄)₂SO₄) to make 1.0 mg/ml solution, as a test sample solution; and the control antibodies (hydrophilic control, Tagitanlimab; hydrophobic control, Sacituzumab. Both are produced by Sichuan Kelun-Biotech Biopharmaceutical Co., Ltd.) were taken respectively and made into a 1 mg/ml solution with diluent as a system suitability solution. About 40 μg of sample was injected; the gradient elution was analyzed; the hydrophobic value of the test sample was calculated according to the control sample after detection, and the calculation formula is: (retention time of the test sample-retention time of the hydrophilic control)/(retention time of the hydrophobic control-retention time of the hydrophilic control), the smaller the retention time and hydrophobic value, the better the hydrophilicity of the antibody. The results are shown in Table 10. The antibody 101A6HZm conjugate had good hydrophilicity, which was equivalent to the Sacituzumab nude antibody, and could improve the metabolism of ADC drugs in vivo.

TABLE 10
hydrophilicity detection of anti-
human PTK7 antibody and conjugate

| | Retention | Hydrophobic |
|---|---|---|
| Antibody | time | value |
| Hydrophilicity control | 9.097 | 0.00 |
| Hydrophobicity control | 15.962 | 1.00 |
| 101A6HZm-A-05 | 15.451 | 0.9 |

### 8. Detection of Binding Activity of Anti-Human PTK7 Antibody-Drug Conjugate to Fc Receptor

ForteBio (Pall life sciences) was adopted to detect the dynamic affinity of the antibodies 101A6HZm and 101A6HZm-A-05 to human Fc receptor proteins CD16a, CD32a, CD32b, CD64, and FcRn. The specific method included: capturing biotinylated proteins to be detected in a PBST solution by using SA sensors (Pall life sciences) respectively; diluting the antibodies and the conjugate to be detected to reach an initial concentration of 5,000 nM by using PBST, and performing double dilution until reaching 7 concentration points; binding, dissociating and opening the detection result in Data Analysis 11.0 software; and analyzing the result by selecting a 1:1 mode and global fitting to obtain the binding rate, dissociation rate and affinity constant; and the result is shown in Table 11.

TABLE 11
Detection of binding activity of antibody
and conjugate to Fc receptor

| | KD(M) | | | | |
|---|---|---|---|---|---|
| Antibody | CD16a | CD32a | CD32b | CD64 | FcRn |
| 101A6HZm | Not | Not | Not | Not | 1.66E−8 |
| | detected | detected | detected | detected | |
| 101A6HZm-A-05 | Not | Not | Not | Not | 1.42E−8 |
| | detected | detected | detected | detected | |

The results show that the mutated 101A6HZm and the conjugate thereof did not bind to the Fc receptor CD16a, CD32a, CD32b, and CD64 proteins, which could reduce the non-specific killing mediated by Fc receptors and improve drug safety. At the same time, 101A6HZm and the conjugates thereof retained the FcRn protein binding activity and did not affect the half-life of the drug.

### 9. In Vivo Efficacy Detection of Anti-Human PTK7 Antibody-Drug Conjugate in HCC1806 Model

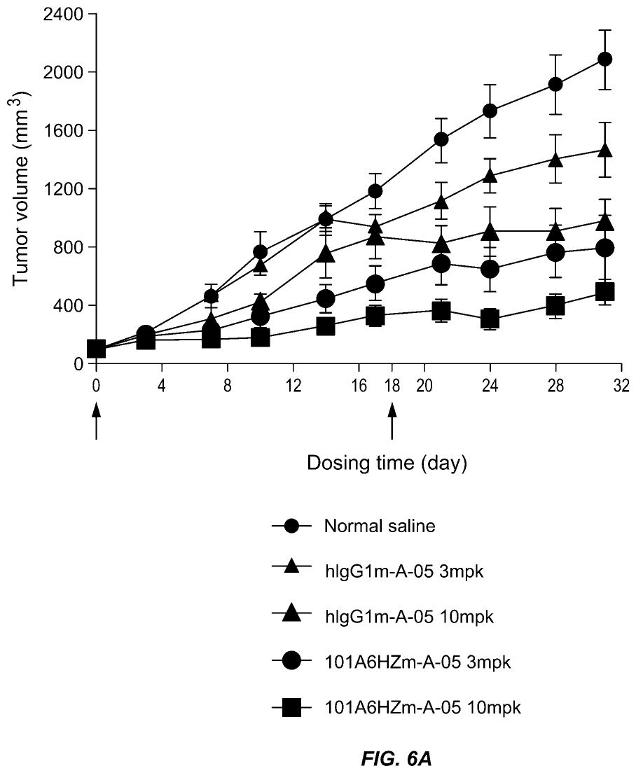

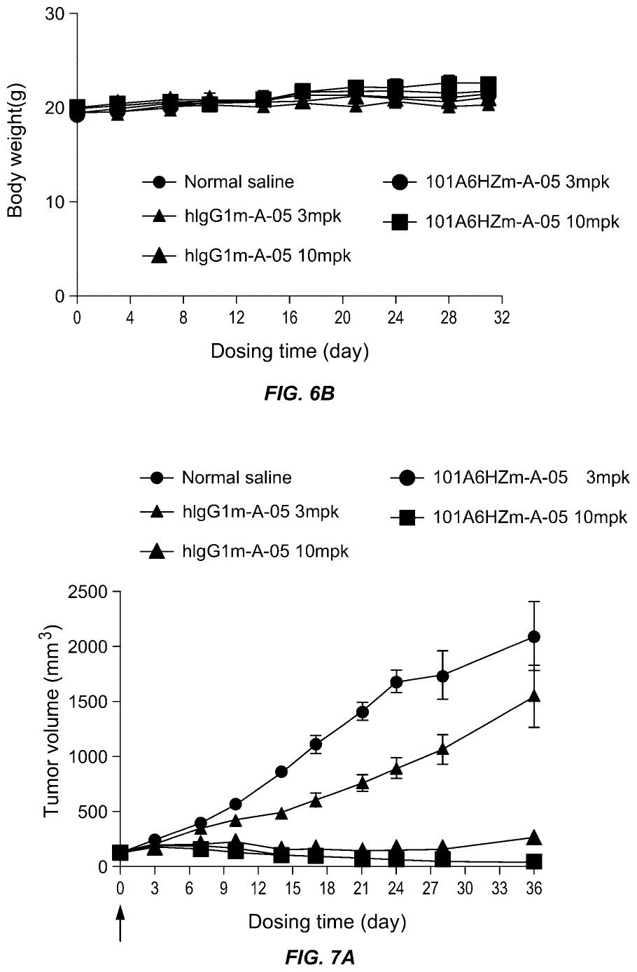

Human breast cancer HCC1806 cells were cultured in a single layer in vitro, and the culture conditions included: 10% of fetal bovine serum was added into a RPMI 1640 medium, and incubation was carried out in an incubator containing air having 5% CO₂ at 37 °C 2×10⁶ HCC1806 cells were inoculated subcutaneously in the right underarm of each mouse, and suspended in 0.1 ml PBS. When the average tumor volume grew to about 100-150 mm³, the mice with too small or too large tumor volume were excluded, and the remaining mice were randomly divided into 5 groups according to the tumor volume and animal weight, with 5 mice in each group; the drug was given IV once every two weeks, twice in total (P0 and P18). The tumor volume and body weight were measured twice a week after dosing, and the specific results are shown in Table 12, and FIGS. 6A-6B.

TABLE 12
Analysis of efficacy of antibody-drug conjugate to human breast cancer
cells in HCC1806 subcutaneous tumor-bearing mouse model (N = 5)

| | Dosing | | | | |
|---|---|---|---|---|---|
| | Dosage | | | | |
| Drug | (mg/kg) | V_{P0}(mm³, mean ± SEM) | V_{P31}(mm³, mean ± SEM) | TGI (%) | P Value |
| Normal saline | / | 100.87 ± 9.32 | 2086.11 ± 202.17 | / | / |
| hIgG1m-A-05 | 3 | 100.47 ± 8.76 | 1470.03 ± 187.50 | 31.01 | 0.0559 |
| hIgG1m-A-05 | 10 | 102.18 ± 9.99 | 979.31 ± 147.87 | 55.82** | 0.0040 |
| 101A6HZm-A-05 | 3 | 100.52 ± 9.40 | 801.28 ± 213.70 | 64.70** | 0.0024 |
| 101A6HZm-A-05 | 10 | 102.08 ± 9.06 | 489.36 ± 83.95 | 80.49*** | 0.0001 |

Note:
T—test,
*P < 0.05,
**P < 0.01,
***P < 0.001 means significant difference compared with normal saline group.

The results showed that the 101A6HZm-A-05 group had significant drug efficacy under the conditions of 3 mg/kg and 10 mg/kg, and the antitumor effect was significantly better than that of the same dose of hIgG1m-A-05 group.

### 10. In Vivo Efficacy Detection of Anti-Human PTK7 Antibody-Drug Conjugate in OVCAR3 Model

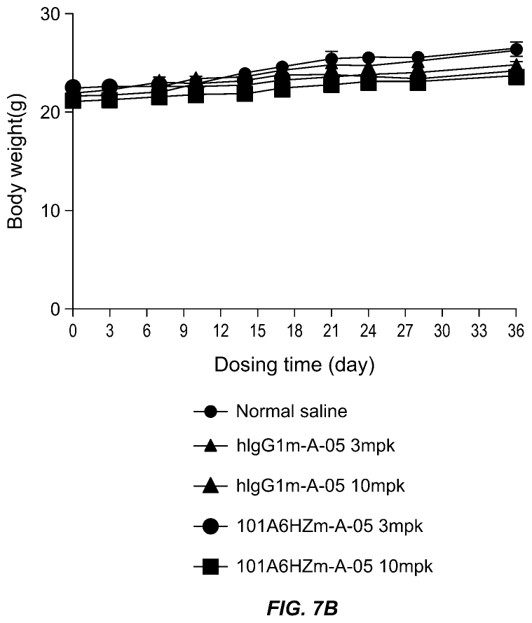

Human ovarian carcinoma OVCAR3 cells were cultured in a single layer in vitro, and the culture conditions included: 20% of fetal bovine serum was added into a RPMI1640 medium, and incubation was carried out in an incubator containing air having 5% CO₂ at 37 °C 5×10⁶ OVCAR3 cells were inoculated subcutaneously in the right underarm of each mouse, and suspended in 0.05 ml PBS+0.05 ml matrigel. When the average tumor volume grew to about 100-150 mm³, the mice with too small or too large tumor volume were excluded, and the remaining mice were randomly divided into 5 groups according to the tumor volume and animal weight, with 5 mice in each group; and the drug was given IV in single dose. The tumor volume and body weight were measured twice a week after dosing, and the specific results are shown in Table 13, and FIGS. 7A-7B.

TABLE 13
Analysis of efficacy of antibody-drug conjugate to human ovarian
carcinoma cells in OVCAR3 subcutaneous tumor-bearing mouse model (N = 5)

| | Dosing | | | | | |
|---|---|---|---|---|---|---|
| | Dosage | V_{P0}(mm³, | V_{P36}(mm³, | TGI | | Tumor |
| Drug | (mg/kg) | mean ± SEM) | mean ± SEM) | (%) | P Value | Remission |
| Normal | / | 131.40 ± 9.19 | 2081.67 ± 316.61 | / | / | None |
| saline | | | | | | |
| hlgG1m-A- | 3 | 130.66 ± 11.60 | 1550.40 ± 281.78 | 27.20 | 0.2454 | None |
| 05 | | | | | | |
| hIgG1m-A- | 10 | 132.09 ± 9.09 | 256.56 ± 21.21 | 93.62*** | 0.0004 | None |
| 05 | | | | | | |
| 101A6HZm- | 3 | 129.39 ± 11.41 | 39.57 ± 10.89 | 169.41*** | 0.0002 | 1/5CR, |
| A-05 | | | | | | 4/5PR |
| 101A6HZm- | 10 | 129.93 ± 8.95 | 45.53 ± 3.11 | 164.96*** | 0.0002 | 5/5PR |
| A-05 | | | | | | |

Note:
T-test,
**P < 0.01,
***P < 0.001 means significant difference compared with normal saline group.

The results showed that the 101A6HZm-A-05 group had significant drug efficacy under the conditions of 3 mg/kg and 10 mg/kg, and the antitumor effect was significantly better than that of the same dose of hIgG1m-A-05 group. In terms of tumor regression, under the condition of 3 mg/kg 101A6HZm-A-05, 1 out of 5 dosed mice had complete response (CR), and 4 partial response (PR); and under the condition of 101A6HZm-A-05 at 10 mg/kg condition, all 5 dosed mice achieved partial response (PR).

### 11. In Vivo Efficacy Detection of Anti-Human PTK7 Antibody-Drug Conjugate in NCI-H146 Model

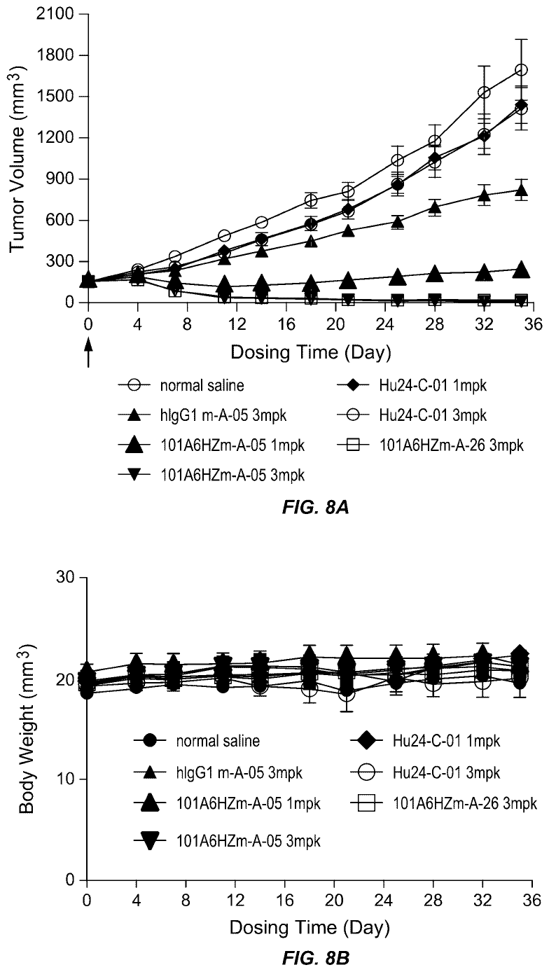

Human small-cell lung carcinoma NCI-H146 cells were cultured in a single layer in vitro, and the culture conditions included: 10% of fetal bovine serum was added into a RPMI 1640 medium, and incubation was carried out in an incubator containing air having 5% CO₂ at 37 °C 5×10⁶ NCI-H146 cells were inoculated subcutaneously in the right underarm of each mouse, and suspended in 0.05 ml PBS+0.05 ml matrigel. When the average tumor volume grew to about 150-200 mm³, the mice with too small or too large tumor volume were excluded, and the remaining mice were randomly divided into 7 groups according to the tumor volume and animal weight, the drug was given in single dose. The tumor volume and body weight were measured twice a week after dosing, and the specific results are shown in Table 14, FIGS. 8A-B.

TABLE 14
Analysis of efficacy of antibody-drug conjugate to NCI-H146 (human small-
cell lung carcinoma cells) subcutaneous tumor-bearing mouse model (N = 5)

| | Dosing | | | | |
|---|---|---|---|---|---|
| | dosage | | | | |
| Drug | (mg/kg) | V_{P0} (mm³, mean ± SEM) | V_{P35} (mm³, mean ± SEM) | TGI (%) | P value |
| Normal saline | / | 153.66 ± 5.36 | 1695.16 ± 219.94 | / | / |
| hIgG1m-A-05 | 3 | 153.83 ± 5.18 | 821.17 ± 77.32 | 56.71 | 0.0056 |
| 101A6HZm-A-05 | 1 | 154.53 ± 7.22 | 244.21 ± 17.27 | 94.18*** | 0.0002 |
| 101A6HZm-A-05 | 3 | 154.52 ± 5.49 | 10.42 ± 3.59 | 193.26*** | 0.0001 |
| Hu24-C-01 | 1 | 154.70 ± 5.60 | 1440.67 ± 136.78 | 16.58 | 0.3885 |
| Hu24-C-01 | 3 | 154.85 ± 5.10 | 1412.09 ± 155.26 | 18.44 | 0.3525 |
| 101A6HZm-A-26 | 3 | 154.04 ± 9.57 | 17.87 ± 2.13 | 188.40*** | 0.0001 |

Note:
T—test,
**P < 0.01,
***P < 0.001 means significant difference compared with normal saline group.

The results shows that the 101A6HZm-A-05 group had significant drug efficacy under the conditions of 1 mg/kg and 3 mg/kg, and the antitumor effect was significantly better than that of the same dose of hIgG1m-A-05 group and Hu24-C-01 group. 101A6HZm-A-05 and 101A6HZm-A-26 has similar drug efficacy under the condition of same dose. Under the conditions of 101A6HZm-A-05 at 3 mg/kg, and 101A6HZm-A-26 at 3 mg/kg, all 5 dosed mice achieved partial response (PR).

Although the specific embodiment of the present invention has been described in detail, those skilled in the art will understand that according to all the teachings disclosed, various modifications and replacements can be made to those details, and these changes are all within the protection scope of the present invention. The full scope of the invention is given by the appended claims and any equivalents thereof.

1-73. (canceled)

74. An antibody-drug conjugate, having a structure shown as a formula Ab-[M-L-E-D]ₓ, wherein:
Ab is an antibody or antigen-binding fragment thereof that specifically binds to human PTK7;
M is a linker site linked to the antibody or antigen-binding fragment thereof;
L is a linker between M and E;
E is a structural fragment linking L and D;
D is a cytotoxic drug fragment; and
x is selected from 1 to 10.

75. The antibody-drug conjugate according to claim 74, wherein the antibody or antigen-binding fragment thereof comprises:
(1) the following heavy chain variable regions (VH) and/or light chain variable regions (VL), wherein CDRs are defined according to a Chothia numbering system:
(1a) a heavy chain variable region (VH) comprising the following 3 CDRs: CDR-H1 having a sequence shown as SEQ ID NO: 11 or a variant thereof, CDR-H2 having a sequence shown as SEQ ID NO: 12 or a variant thereof, and CDR-H3 having a sequence shown as SEQ ID NO: 13 or a variant thereof; and/or, a light chain variable region (VL) comprising the following 3 CDRs: CDR-L1 having a sequence shown as SEQ ID NO: 14 or a variant thereof, CDR-L2 having a sequence shown as SEQ ID NO: 15 or a variant thereof, and CDR-L3 having a sequence shown as SEQ ID NO: 16 or a variant thereof; or,
(1b) a heavy chain variable region (VH) comprising the following 3 CDRs: CDR-H1 having a sequence shown as SEQ ID NO: 27 or a variant thereof, CDR-H2 having a sequence shown as SEQ ID NO: 28 or a variant thereof, and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof, CDR-L2 having a sequence shown as SEQ ID NO: 31 or a variant thereof, and CDR-L3 having a sequence shown as SEQ ID NO: 32 or a variant thereof;
where the variants in any one of (1a) and (1b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids compared with the sequence from which the variants are derived;
or,
(2) the following heavy chain variable regions (VH) and/or light chain variable regions (VL), wherein the CDRs are defined according to the Kabat numbering system:
(2a) a heavy chain variable region (VH) comprising the following 3 CDRs: CDR-H1 having a sequence shown as SEQ ID NO: 17 or a variant thereof, CDR-H2 having a sequence shown as SEQ ID NO: 18 or 19 or a variant thereof, and CDR-H3 having a sequence shown as SEQ ID NO: 13 or a variant thereof; and/or, a light chain variable region (VL) comprising the following 3 CDRs: CDR-L1 having a sequence shown as SEQ ID NO: 14 or a variant thereof, CDR-L2 having a sequence shown as SEQ ID NO: 15 or a variant thereof, and CDR-L3 having a sequence shown as SEQ ID NO: 16 or a variant thereof, or,
(2b) a heavy chain variable region (VH) comprising the following 3 CDRs: CDR-H1 having a sequence shown as SEQ ID NO: 33 or a variant thereof, CDR-H2 having a sequence shown as SEQ ID NO: 34 or 35 or a variant thereof, and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof, CDR-L2 having a sequence shown as SEQ ID NO: 31 or a variant thereof, and CDR-L3 having a sequence shown as SEQ ID NO: 32 or a variant thereof;
where the variants in any one of (2a) and (2b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids compared with the sequence from which the variants are derived;
or,
(3) the following heavy chain variable regions (VH) and/or light chain variable regions (VL), wherein the CDRs are defined according to the IMGT numbering system:
(3a) a heavy chain variable region (VH) comprising the following 3 CDRs: CDR-H1 having a sequence shown as SEQ ID NO: 20 or a variant thereof, CDR-H2 having a sequence shown as SEQ ID NO: 21 or a variant thereof, and CDR-H3 having a sequence shown as SEQ ID NO: 22 or a variant thereof; and/or, a light chain variable region (VL) comprising the following 3 CDRs: CDR-L1 having a sequence shown as SEQ ID NO: 23 or a variant thereof, CDR-L2 having a sequence shown as SEQ ID NO: 24 or a variant thereof, and CDR-L3 having a sequence shown as SEQ ID NO: 16 or a variant thereof, or,
(3b) a heavy chain variable region (VH) comprising the following 3 CDRs: CDR-H1 having a sequence shown as SEQ ID NO: 36 or a variant thereof, CDR-H2 having a sequence shown as SEQ ID NO: 37 or a variant thereof, and CDR-H3 having a sequence shown as SEQ ID NO: 38 or a variant thereof; and/or, a light chain variable region (VL) comprising the following 3 CDRs: CDR-L1 having a sequence shown as SEQ ID NO: 39 or a variant thereof, CDR-L2 having a sequence shown as SEQ ID NO: 40 or a variant thereof, and CDR-L3 having a sequence shown as SEQ ID NO: 32 or a variant thereof;
where the variants in any one of (3a) and (3b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids compared with the sequence from which the variants are derived;
or,
(4) the following heavy chain variable regions (VH) and/or light chain variable regions (VL), wherein the CDRs are defined according to the AbM numbering system:
(4a) a heavy chain variable region (VH) comprising the following 3 CDRs: CDR-H1 having a sequence shown as SEQ ID NO: 25 or a variant thereof, CDR-H2 having a sequence shown as SEQ ID NO: 26 or a variant thereof, and CDR-H3 having a sequence shown as SEQ ID NO: 13 or a variant thereof; and/or, a light chain variable region (VL) comprising the following 3 CDRs: CDR-L1 having a sequence shown as SEQ ID NO: 14 or a variant thereof, CDR-L2 having a sequence shown as SEQ ID NO: 15 or a variant thereof, and CDR-L3 having a sequence shown as SEQ ID NO: 16 or a variant thereof, or,
(4b) a heavy chain variable region (VH) comprising the following 3 CDRs: CDR-H1 having a sequence shown as SEQ ID NO: 41 or a variant thereof, CDR-H2 having a sequence shown as SEQ ID NO: 42 or a variant thereof, and CDR-H3 having a sequence shown as SEQ ID NO: 29 or a variant thereof; and/or, a light chain variable region (VL) comprising the following 3 CDRs: CDR-L1 having a sequence shown as SEQ ID NO: 30 or a variant thereof, CDR-L2 having a sequence shown as SEQ ID NO: 31 or a variant thereof, and CDR-L3 having a sequence shown as SEQ ID NO: 32 or a variant thereof;
where the variants in any one of (4a) and (4b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids compared with the sequence from which the variants are derived.

76. The antibody-drug conjugate according to claim 74, wherein the antibody or antigen-binding fragment thereof comprises:
(a) a VH shown as SEQ ID NO: 1 or a variant thereof, and/or, a VL shown as SEQ ID NO: 2 or a variant thereof;
(b) a VH shown as SEQ ID NO: 3 or a variant thereof, and/or, a VL shown as SEQ ID NO: 4 or a variant thereof;
(c) a VH shown as SEQ ID NO: 5 or a variant thereof, and/or, a VL shown as SEQ ID NO: 6 or a variant thereof;
(d) a VH shown as SEQ ID NO: 7 or a variant thereof, and/or, a VL shown as SEQ ID NO: 8 or a variant thereof; or
(e) a VH shown as SEQ ID NO: 9 or a variant thereof, and/or, a VL shown as SEQ ID NO: 10 or a variant thereof;
where the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids as compared to the sequence from which the variants are derived.

77. The antibody-drug conjugate according to claim 74, wherein the antibody or antigen-binging fragment thereof further comprises:
(a) a human immunoglobulin heavy chain constant region (CH) or a variant thereof, wherein the variant has replacement, deletion or addition of one or more amino acids as compared to a wild-type sequence from which the variant is derived; and
(b) a human immunoglobulin light chain constant region (CL) or a variant, wherein the variant has replacement, deletion or addition of one or more amino acids as compared to a wild-type sequence from which the variant is derived.

78. The antibody-drug conjugate according to claim 74, wherein the antibody or antigen-binding fragment thereof comprises:
(1) a heavy chain comprising VH sequence shown as SEQ ID NO: 1 and heavy chain constant region (CH) shown as SEQ ID NO: 43, and a light chain comprising VL sequence shown as SEQ ID NO: 2 and light chain constant region (CL) shown as SEQ ID NO: 44;
(2) a heavy chain comprising VH sequence shown as SEQ ID NO: 3 and heavy chain constant region (CH) shown as SEQ ID NO: 43 or 45, and a light chain comprising VL sequence shown as SEQ ID NO: 4 and light chain constant region (CL) shown as SEQ ID NO: 44;
(3) a heavy chain comprising VH sequence shown as SEQ ID NO: 5 and heavy chain constant region (CH) shown as SEQ ID NO: 43, and a light chain comprising VL sequence shown as SEQ ID NO: 6 and light chain constant region (CL) shown as SEQ ID NO: 44;
(4) a heavy chain comprising VH sequence shown as SEQ ID NO: 7 and heavy chain constant region (CH) shown as SEQ ID NO: 43, and a light chain comprising VL sequence shown as SEQ ID NO: 8 and light chain constant region (CL) shown as SEQ ID NO: 44; or
(5) a heavy chain comprising VH sequence shown as SEQ ID NO: 9 and heavy chain constant region (CH) shown as SEQ ID NO: 43, and a light chain comprising VL sequence shown as SEQ ID NO: 10 and light chain constant region (CL) shown as SEQ ID NO: 44.

79. The antibody-drug conjugate according to claim 74, wherein M is selected from a group consisting of: 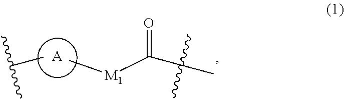
wherein ring A is a 5-6 membered aliphatic heterocyclic ring, or 5-20 membered aromatic ring system, and the aliphatic heterocyclic ring and the aromatic ring system are optionally substituted with one or more members selected from the group consisting of oxo (═O), halogen, cyano, amino, carboxyl, mercapto and C₁₋₆ alkyl; and M₁ is selected from single bond, C₁₋₂₀ alkylene, C₂₋₂₀ alkenylene and C₂₋₂₀ alkynylene; 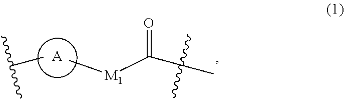
wherein ring A is a 5-membered aliphatic heterocyclic ring, a 6-membered aromatic heterocyclic ring, or a polycyclic ring formed by linking more than one 6-membered aromatic heterocyclic ring and a benzene ring through a single bond, and the aliphatic heterocyclic ring is optionally substituted with one or more members selected from the group consisting of oxo (═O), halogen and C₁₋₄ alkyl; and M₁ is selected from a single bond, C₃₋₁₀ alkylene, C₃₋₁₀ alkenylene and C₃₋₁₀ alkynylene; 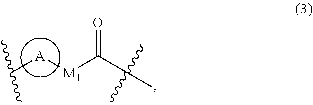
wherein ring A is selected from 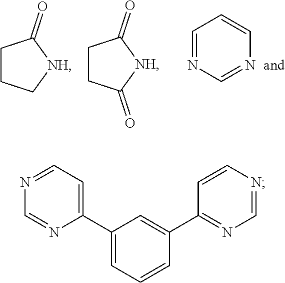
and M₁ is selected from a single bond, C₅₋₈ alkylene, C₅₋₈ alkenylene and C₅₋₈ alkynylene;
(4) a structure selected from

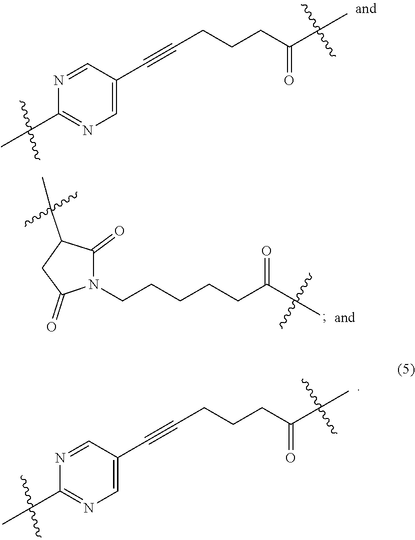

80. The antibody-drug conjugate according to claim 74, wherein L is selected from a group consisting of:
(1) a structure comprising one or more of C₁₋₆ alkylene, —N(R′)—, carbonyl, —O—, Val, Cit, Phe, Lys, Lys(COCH₂CH₂(OCH₂CH₂)ₛOCH₃), D-Val, Leu, Gly, Ala, Asn, Val-Cit, Val-Ala, Val-Lys, Val-Lys(Ac), Phe-Lys, Phe-Lys(Ac), D-Val-Leu-Lys, Gly-Gly-Arg, Ala-Ala-Asn, Ala-Ala-Ala, Val-Lys-Ala, Val-Lys-Gly, Gly-Gly-Gly, Gly-Gly-Phe-Gly (SEQ ID NO: 48), Gly-Phe-Leu-Gly (SEQ ID NO: 49), Gly-Gly-Val-Ala (SEQ ID NO: 50), Gly-Gly-Gly-Gly-Gly (SEQ ID NO: 51), 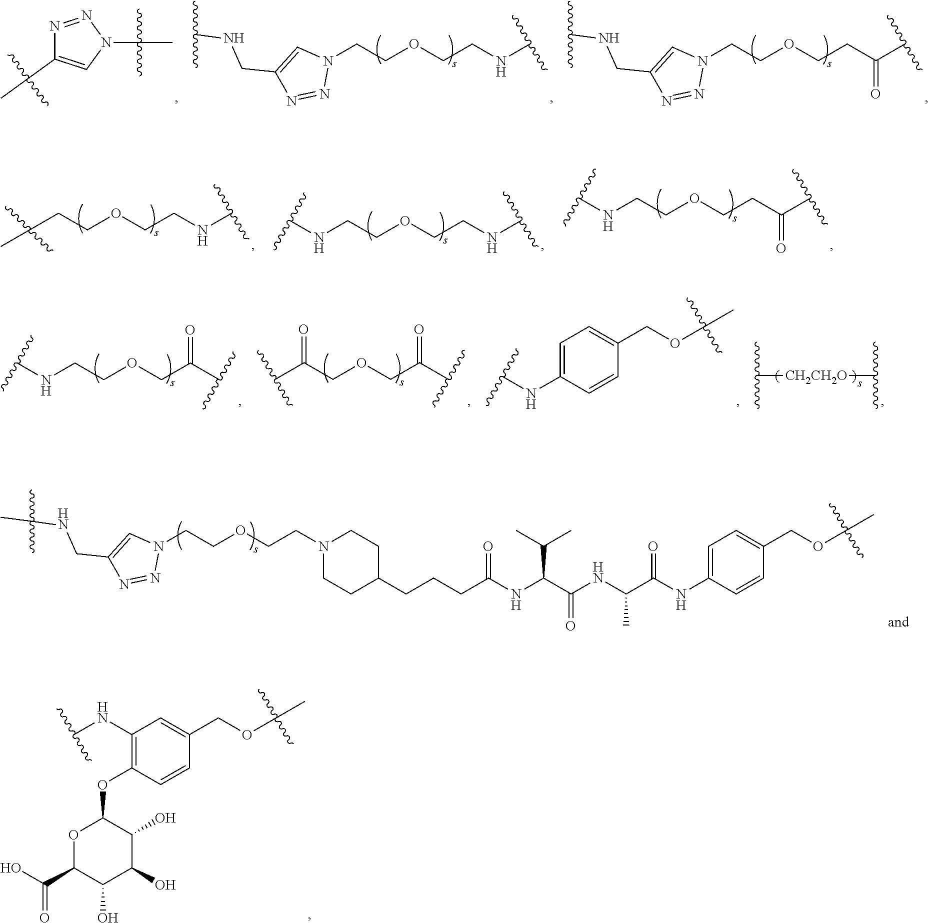
wherein R′ represents hydrogen, C₁₋₆ alkyl or alkyl containing —(CH₂CH₂O)r-; r is an integer selected from 1-10; and s is an integer selected from 1-20;
(2) a structure comprising one or more of C₁₋₆ alkylene, —NH—, Val, Cit, Phe, Lys, Lys(COCH₂CH₂(OCH₂CH₂)ₛOCH₃), Gly, Val-Cit, Gly-Gly-Phe-Gly (SEQ ID NO: 48), 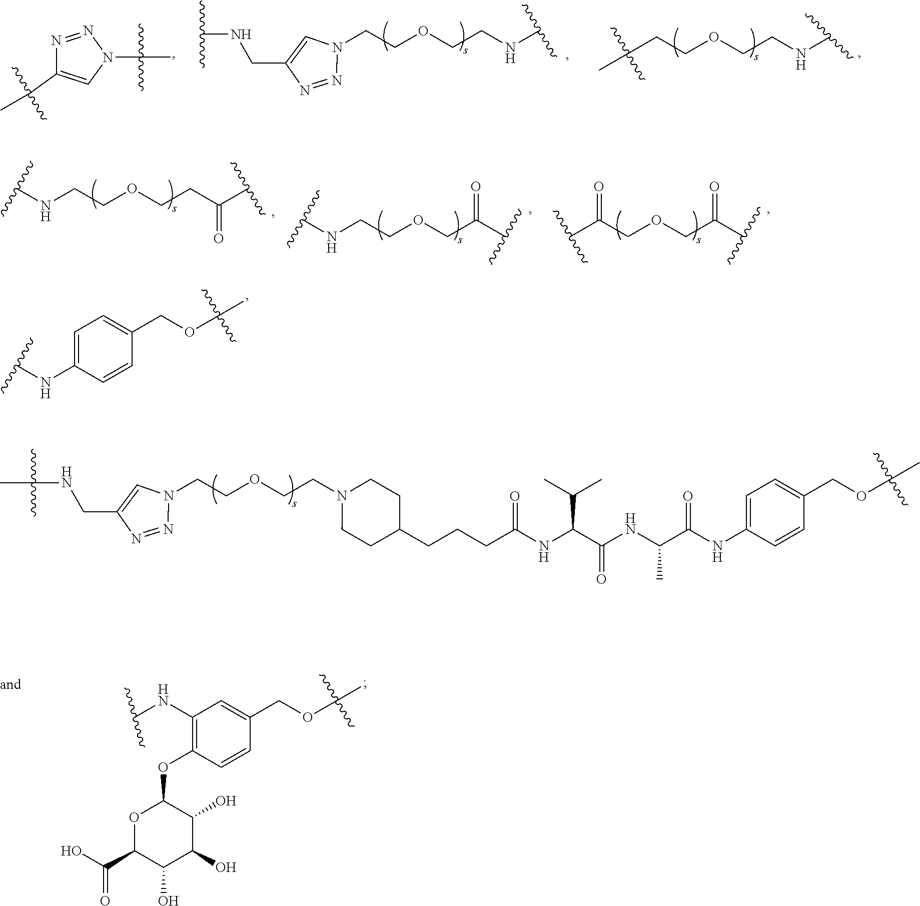
and s is an integer selected from 1-20;
(3) a structure selected from 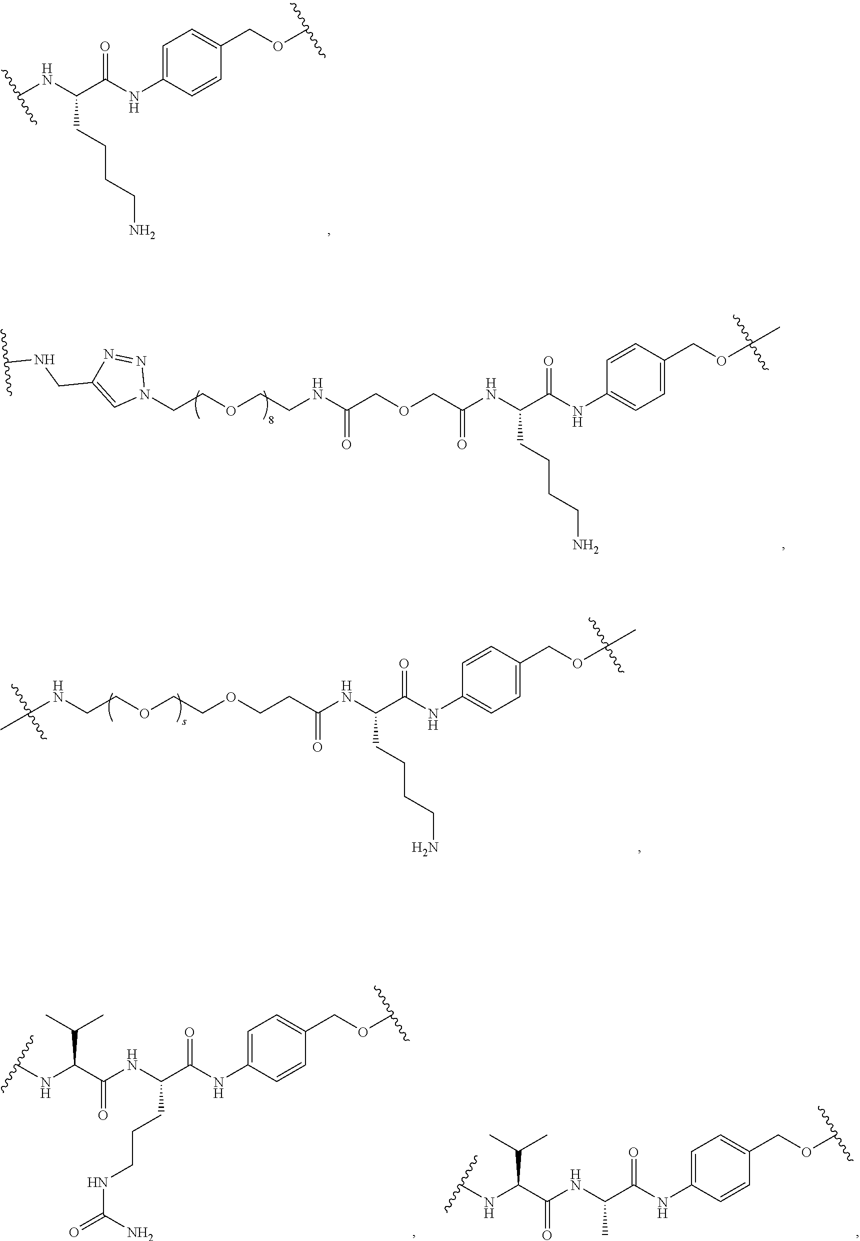 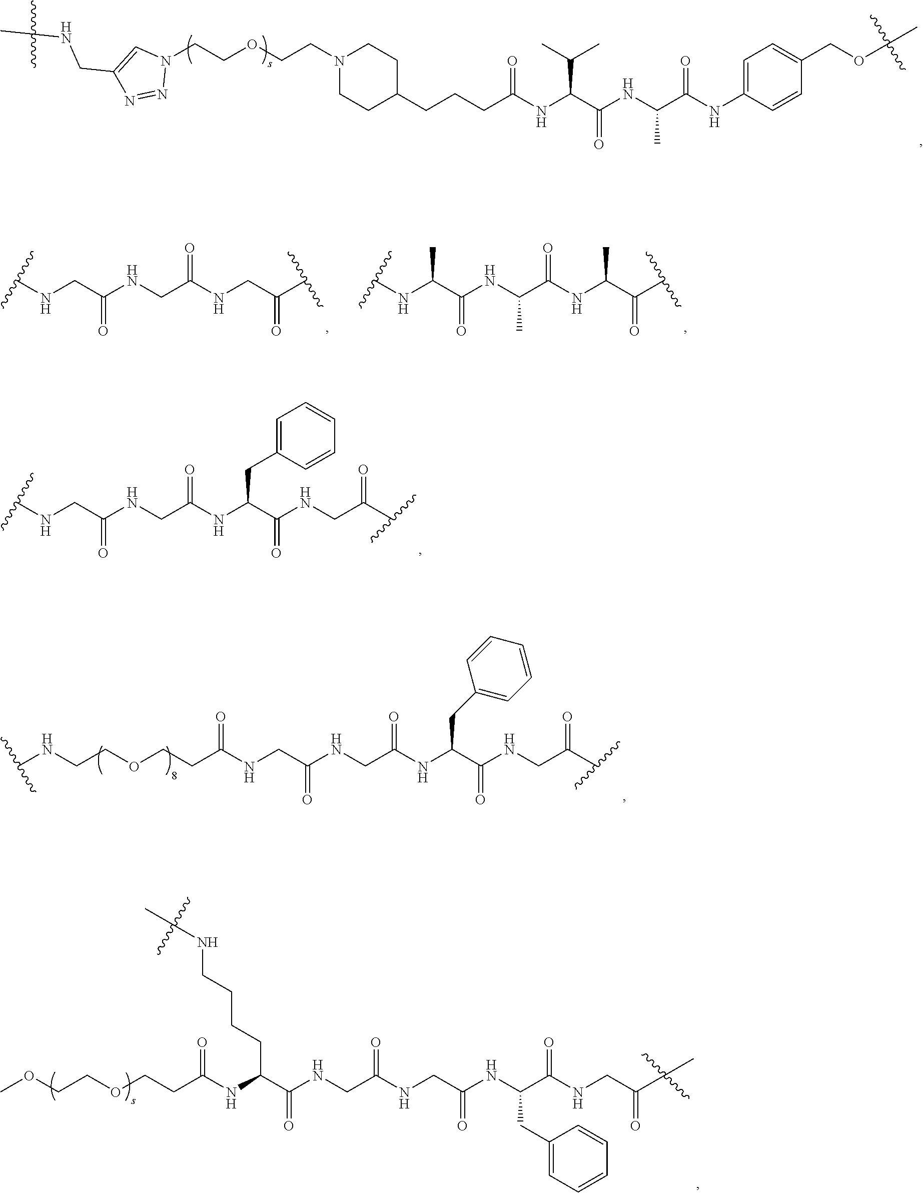 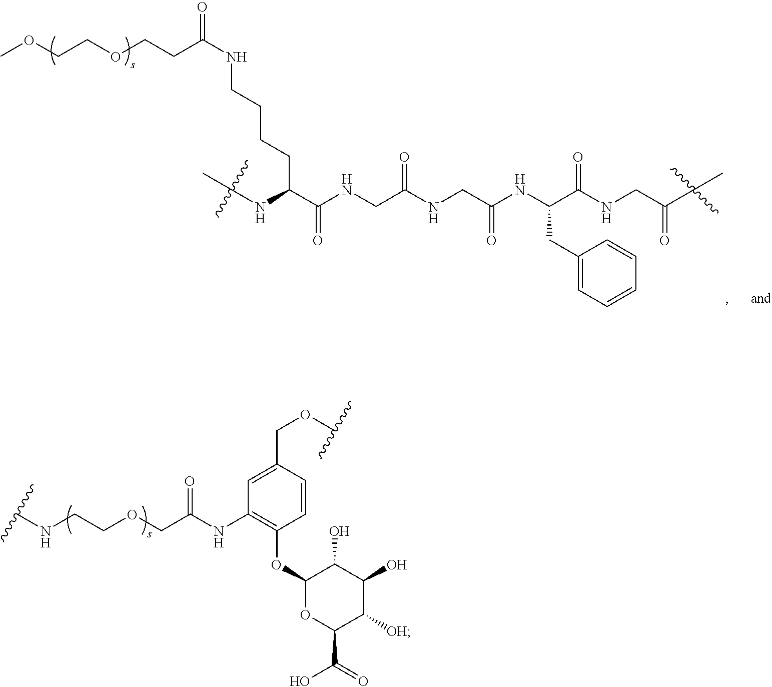
s is an integer selected from 1-20;
(4) a structure selected from 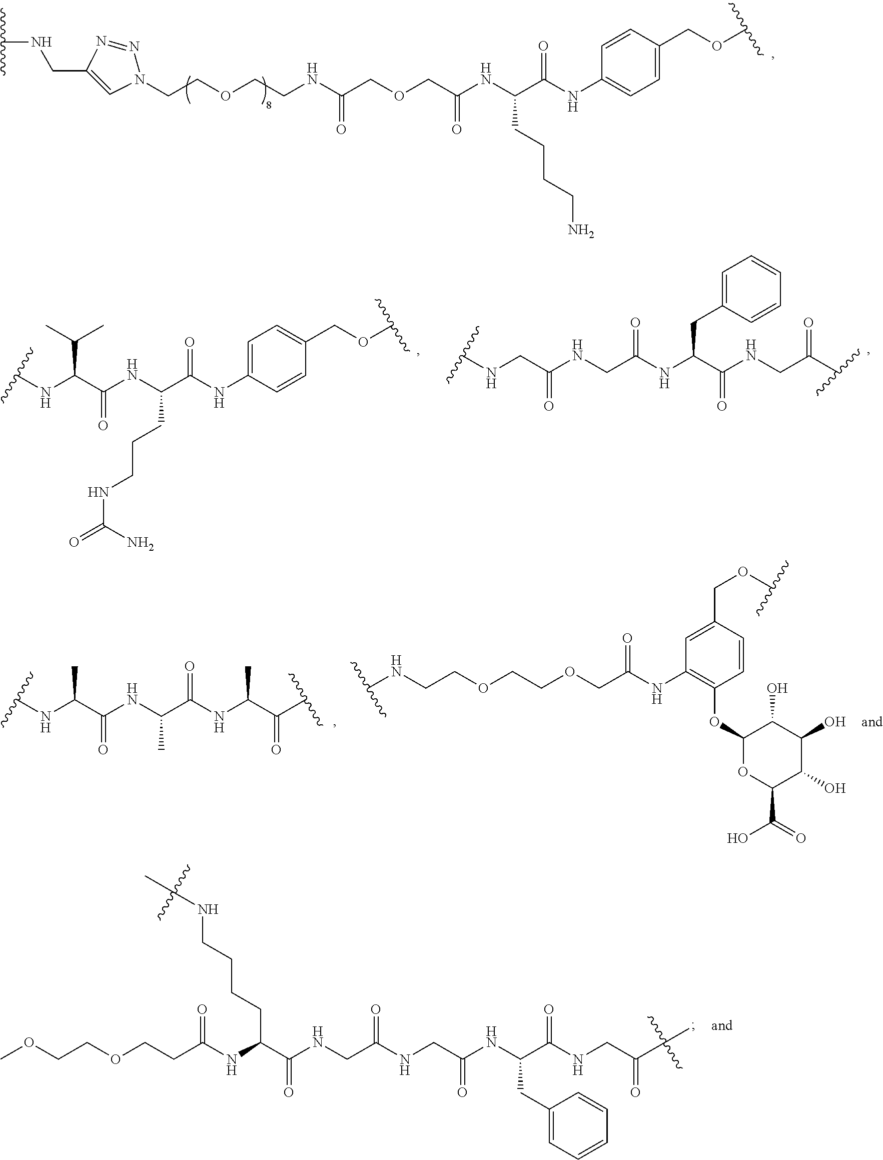
(5) a structure selected from

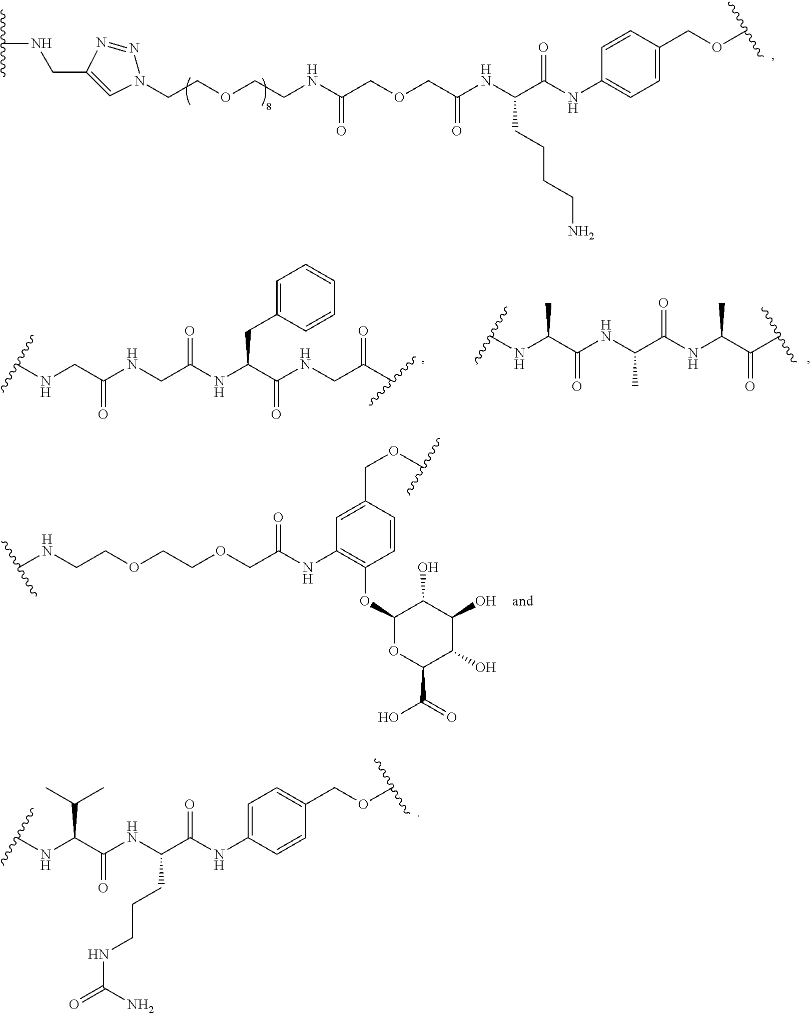

81. The antibody-drug conjugate according to claim 74, wherein E is selected from a group consisting of:
(1) a structure selected from a single bond, —NH—CH₂—, —NH—CH₂—O—CH₂—CO—, 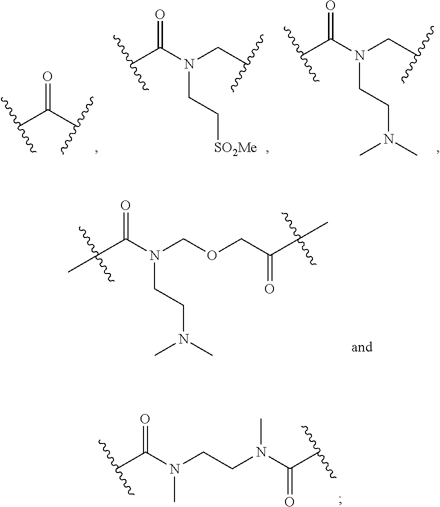
(2) a structure selected from a single bond, —NH—CH₂—, —NH—CH₂—O—CH₂—CO—, 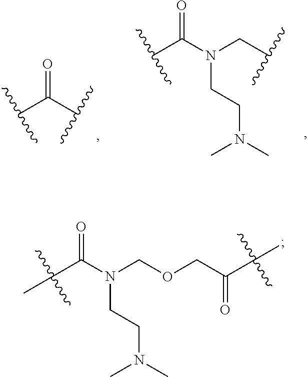
(3) a structure selected from —NH—CH₂—, 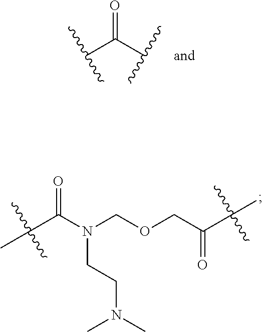
and
(4) a structure selected from —NH—CH₂— and

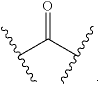 [SMILES: *C(*)=O]

82. The antibody-drug conjugate according to claim 74, wherein 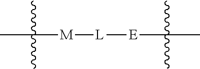
is selected from the following structures:

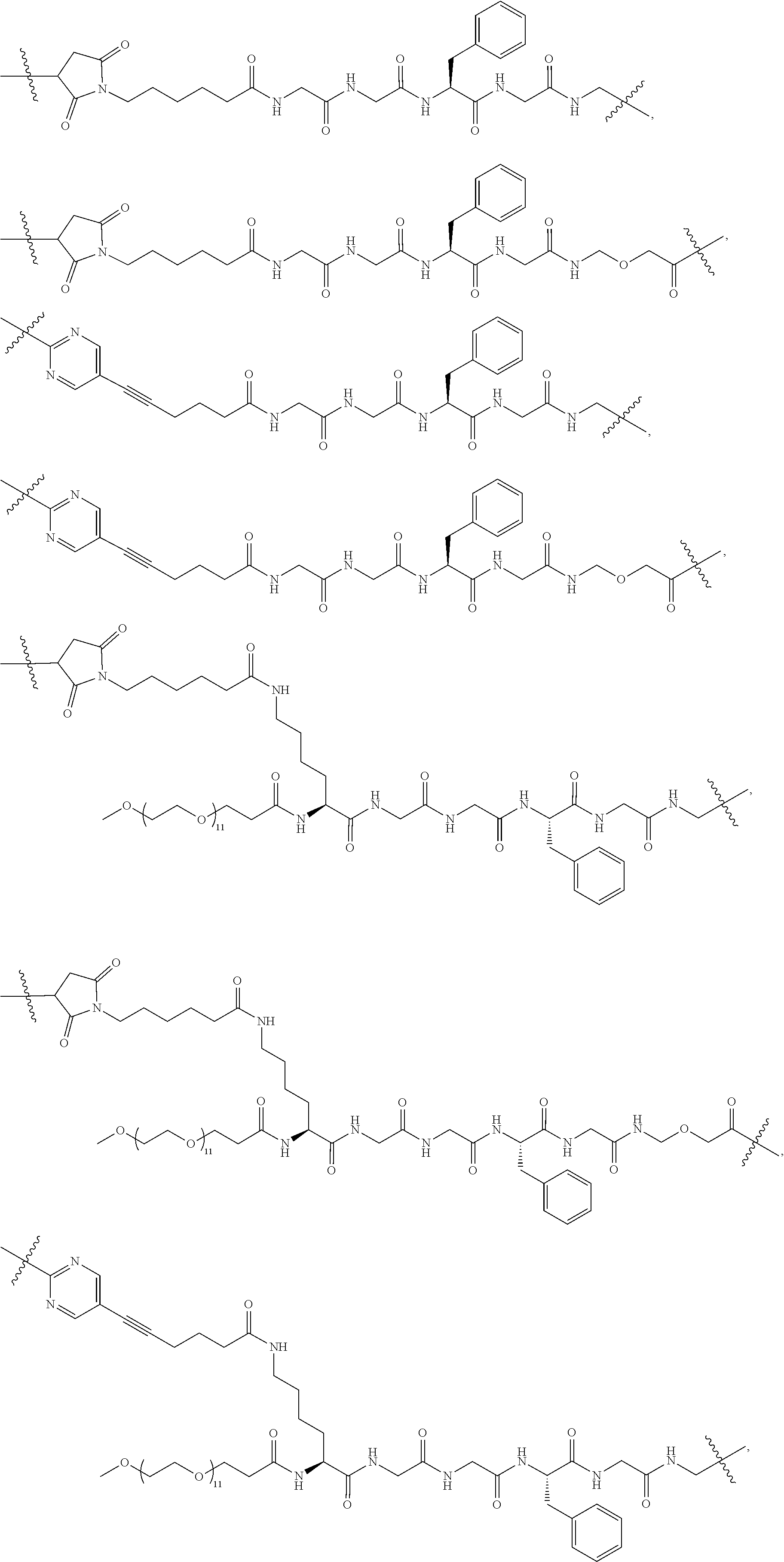

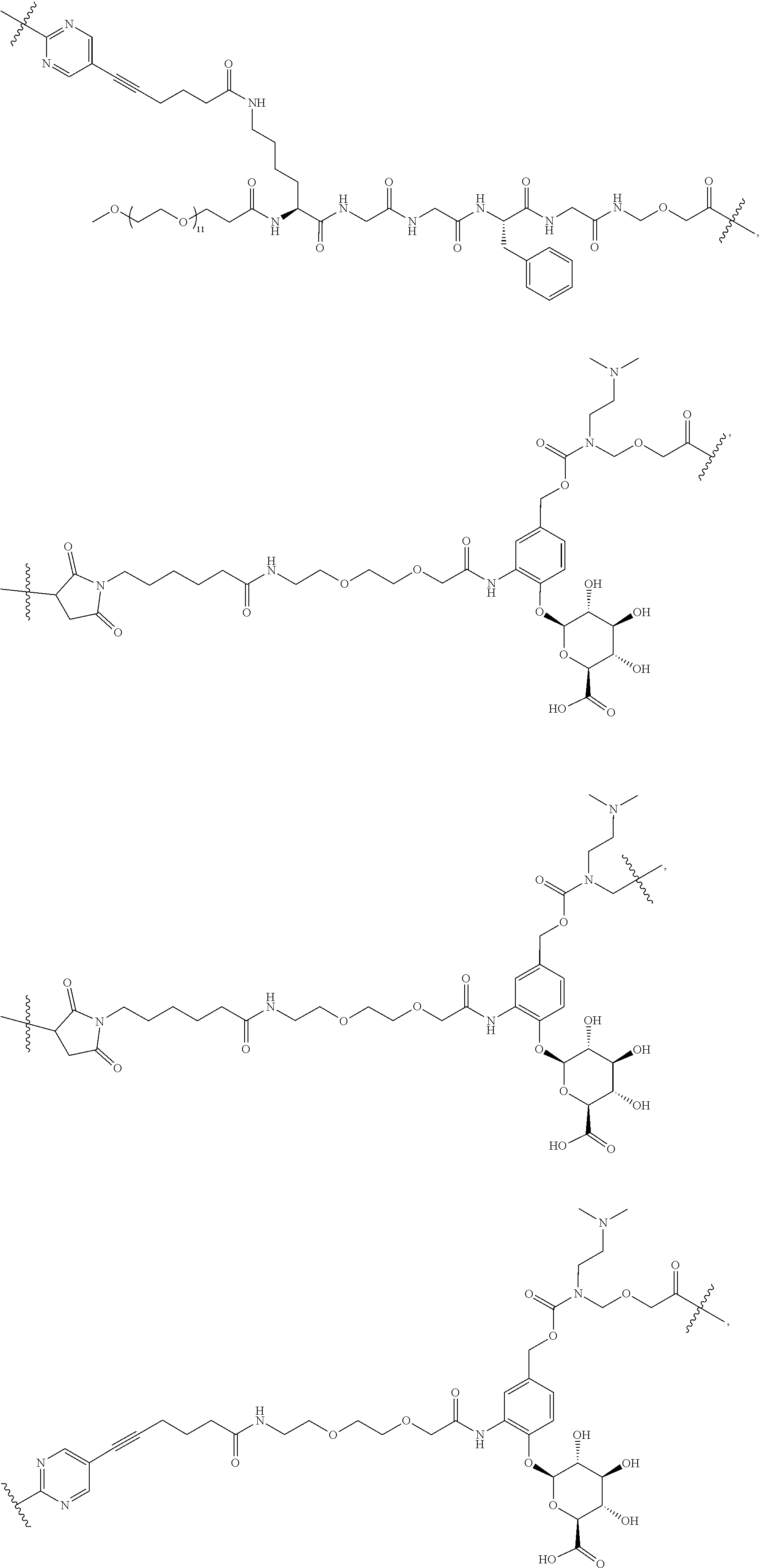

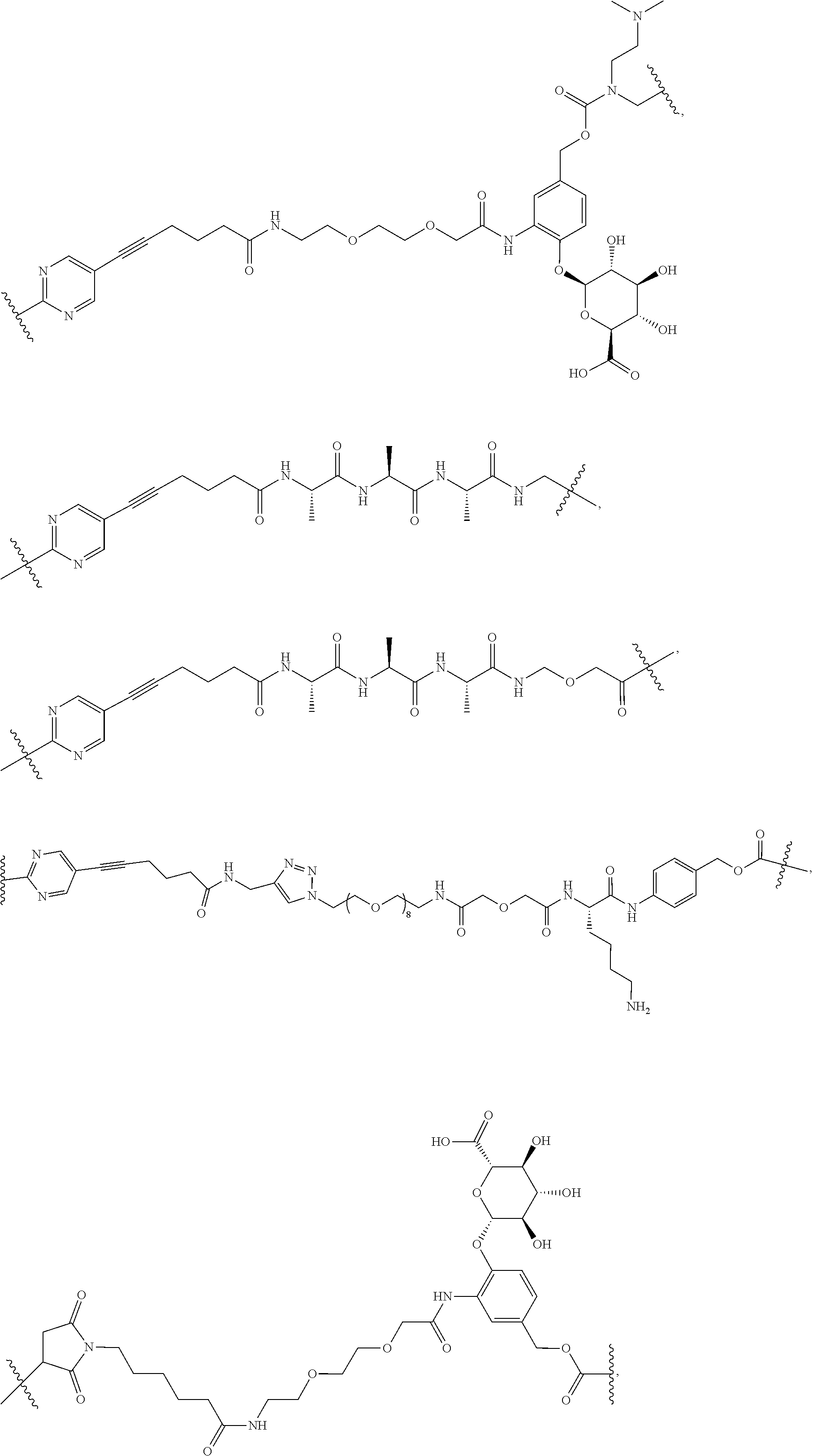

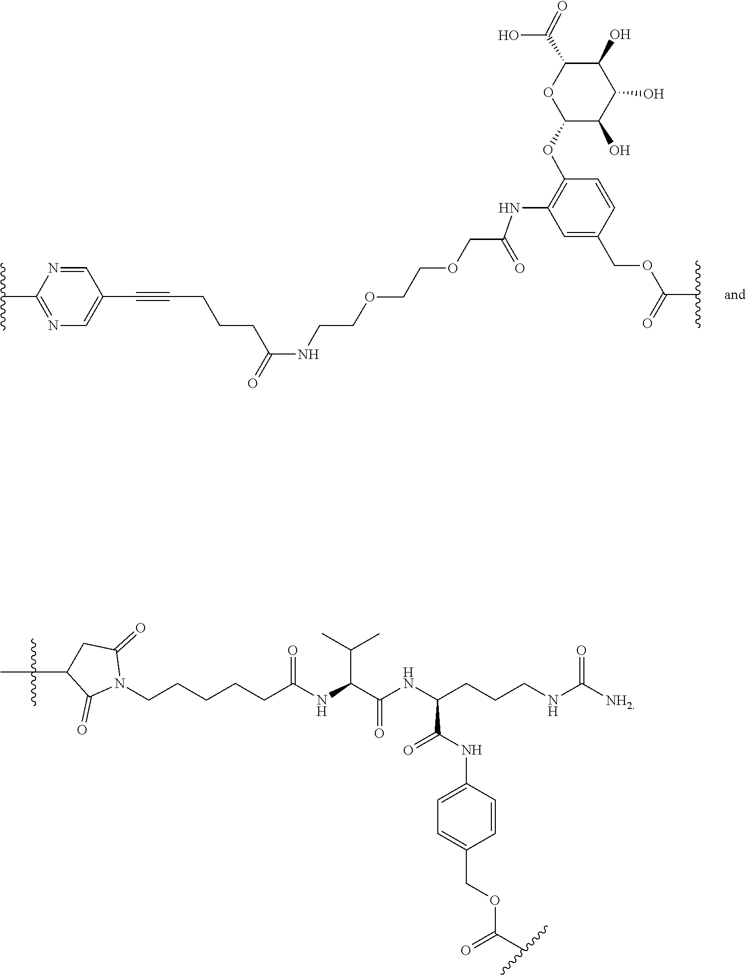 [SMILES: *C(=O)OCc1ccc(O[C@@H]2O[C@H](C(=O)O)[C@@H](O)[C@H](O)[C@H]2O)c(NC(=O)COCCOCCNC(=O)CCCC#Cc2cnc(*)nc2)c1 ; *C(=O)OCc1ccc(NC(=O)[C@H](CCCNC(N)=O)NC(=O)[C@@H](NC(=O)CCCCCN2C(=O)CC(*)C2=O)C(C)C)cc1]

83. The antibody-drug conjugate according to claim 74, wherein the cytotoxic drug is selected from a tubulin inhibitor, a DNA intercalator, a DNA topoisomerase inhibitor and a RNA polymerase inhibitor or pharmaceutically acceptable salt, ester or analog thereof, or the cytotoxic drug is a tubulin inhibitor wherein the tubulin inhibitor is an auristatin compound or a maytansinoid compound;
or the cytotoxic drug is a DNA intercalator wherein the DNA intercalator is pyrrolobenzodiazepine (PBD);
or the cytotoxic drug is a DNA topoisomerase inhibitor wherein the DNA topoisomerase inhibitor is a topoisomerase I inhibitor or a topoisomerase II; and/or
the RNA polymerase inhibitor is a-amanitin or a pharmaceutically acceptable salt, ester or analog thereof;
or, the cytotoxic drug is 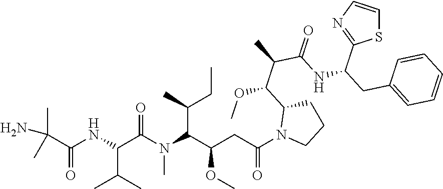
or the cytotoxic drug is selected from a compound shown as formula I and formula II: 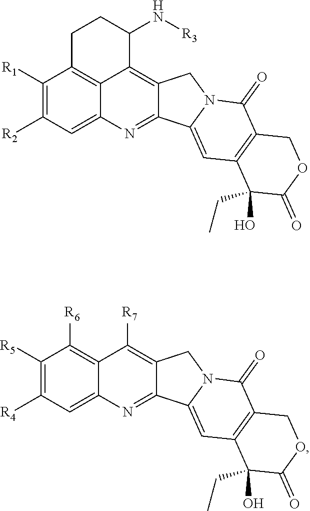
R₁ and R₂ are each independently selected from C₁₋₆ alkyl and halogen;
R₃ is selected from H and —CO—CH₂OH;
R₄ and R₅ are each independently selected from H, halogen and hydroxyl; or R₄ and R₅ are linked to associated carbon atoms to form 5-6 membered oxygen-containing heterocyclic ring;
R₆ is selected from hydrogen and —C₁₋₄ alkylene —NRₐR_{b};
R₇ is selected from hydrogen, C₁₋₆ alkyl and —C₁₋₄ alkylene —NRₐR_{b}; and
Rₐ and R_{b} are each independently selected from H, C₁₋₆ alkyl, —SO₂—C₁₋₆ alkyl and —CO—C₁₋₆ alkyl at each occurrence;
or, the cytotoxic drug is selected from the following compounds: 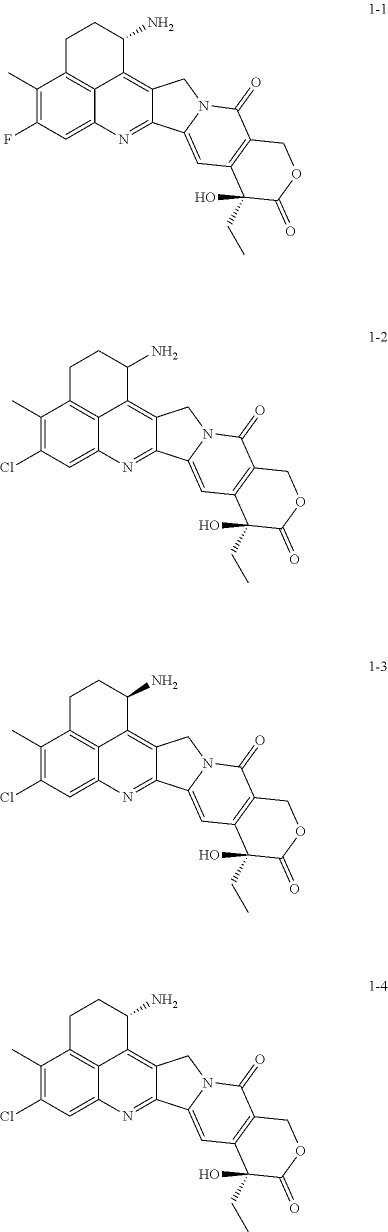 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3] 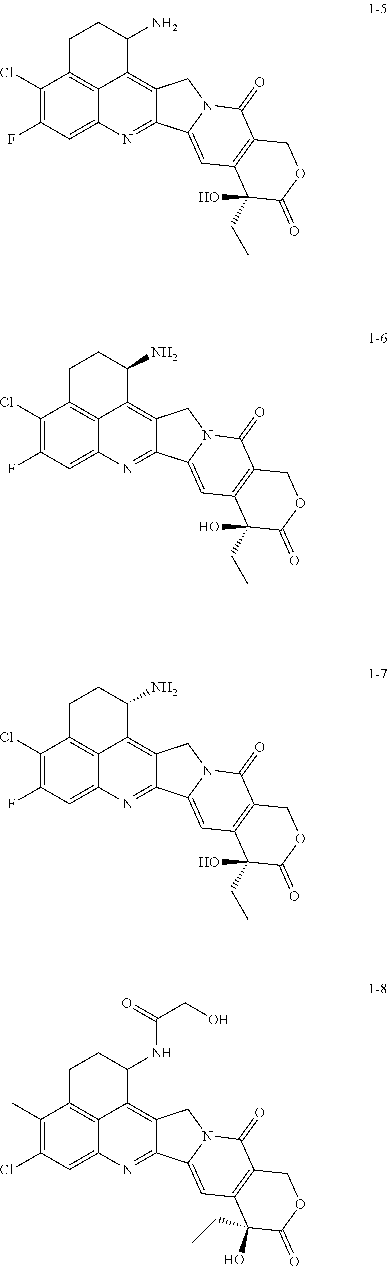 [SMILES: CC[C@@]1(O)C(=O)OCc2c1cc1n(c2=O)Cc2c-1nc1cc(F)c(Cl)c3c1c2C(N)CC3 ; CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3] 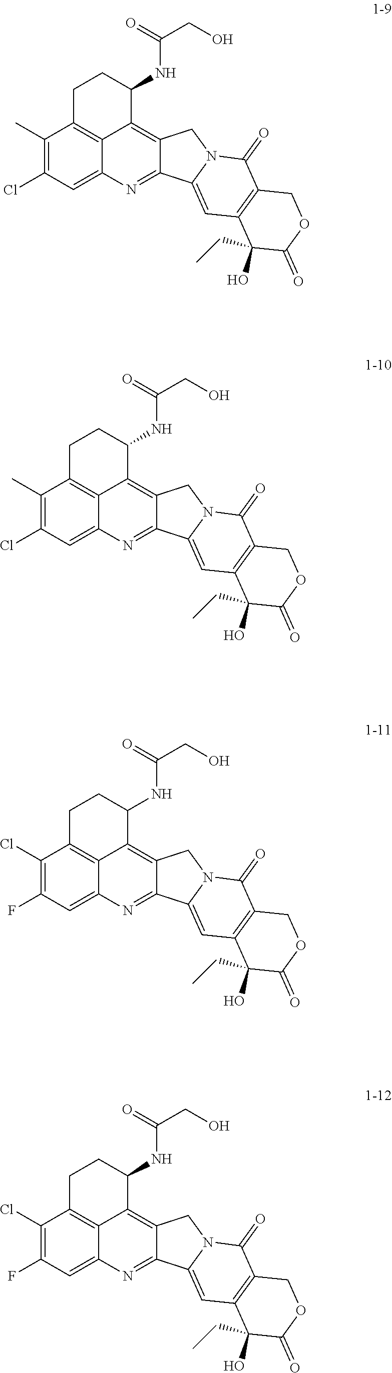 [SMILES: CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3 ; CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3] 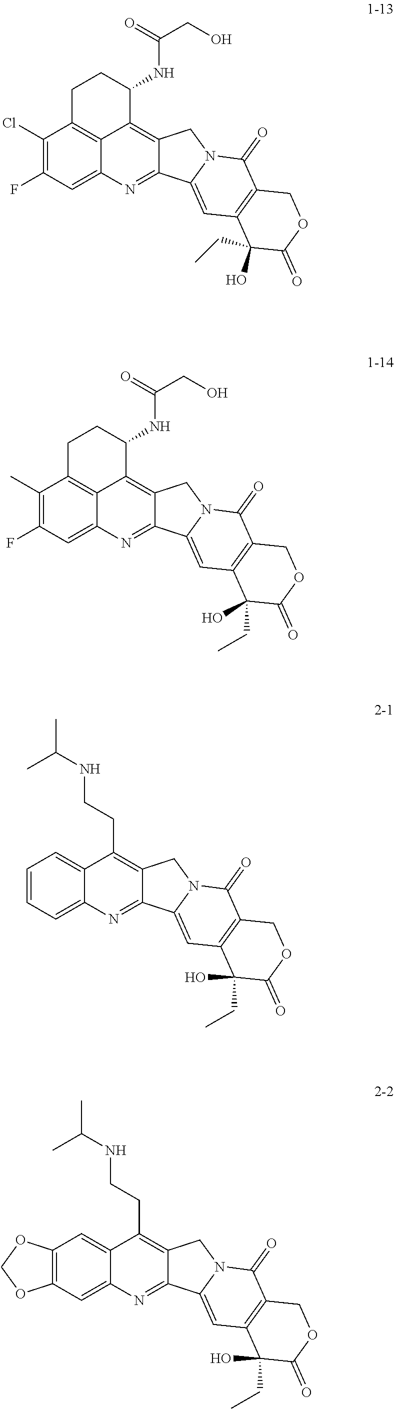 [SMILES: CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3 ; CC[C@@]1(O)C(=O)OCc2c1cc1n(c2=O)Cc2c-1nc1ccccc1c2CCNC(C)C ; CC[C@@]1(O)C(=O)OCc2c1cc1n(c2=O)Cc2c-1nc1cc3c(cc1c2CCNC(C)C)OCO3] 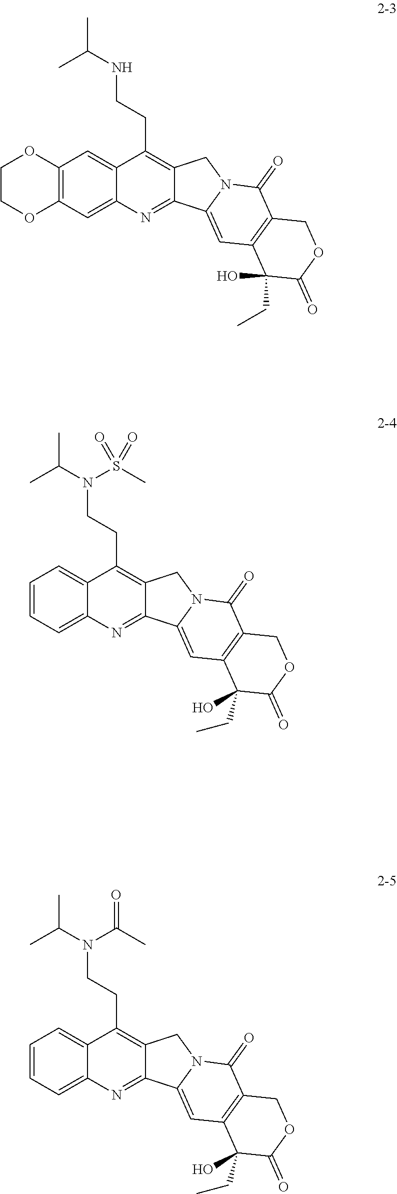 [SMILES: CC[C@@]1(O)C(=O)OCc2c1cc1n(c2=O)Cc2c-1nc1cc3c(cc1c2CCNC(C)C)OCCO3 ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C] 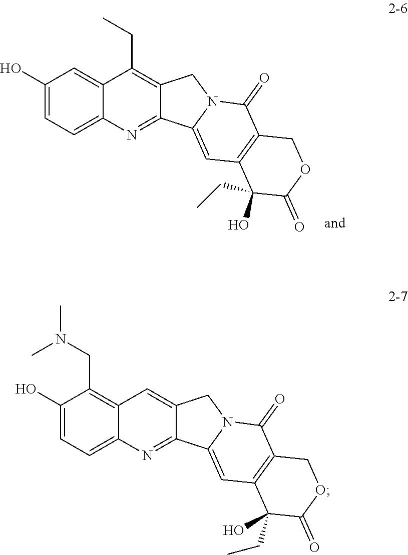 [SMILES: CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2cc3c(CN(C)C)c(O)ccc3nc2-1]
wherein the corresponding fragment of the cytotoxic drug obtained after the cytotoxic drug is linked to the linker is D as shown in the general formula.

84. The antibody-drug conjugate according to claim 74, selected from ADC A-01 to ADC A-26, ADC B-01 to ADC B-06, and ADC C₀₁ shown as follows: 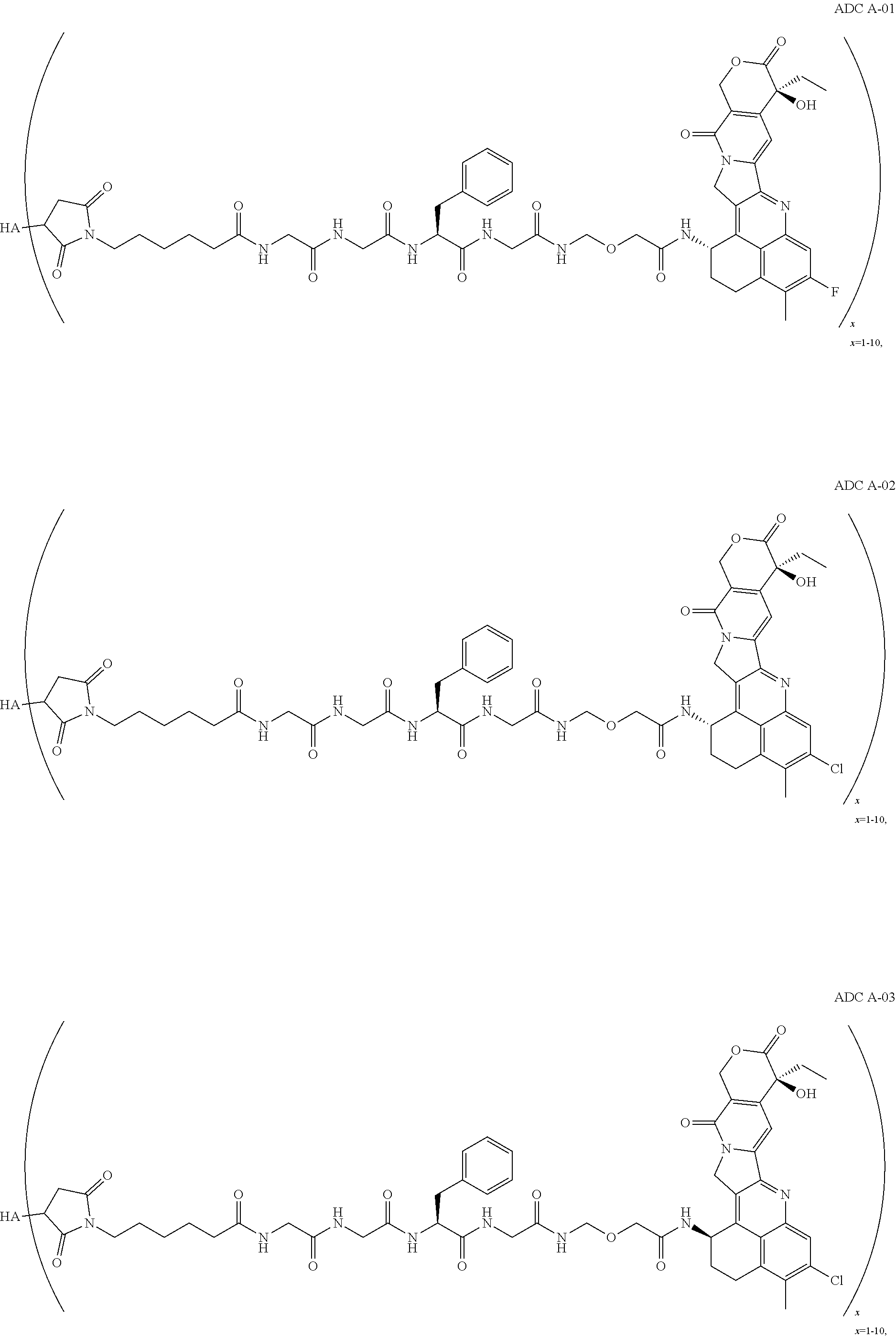 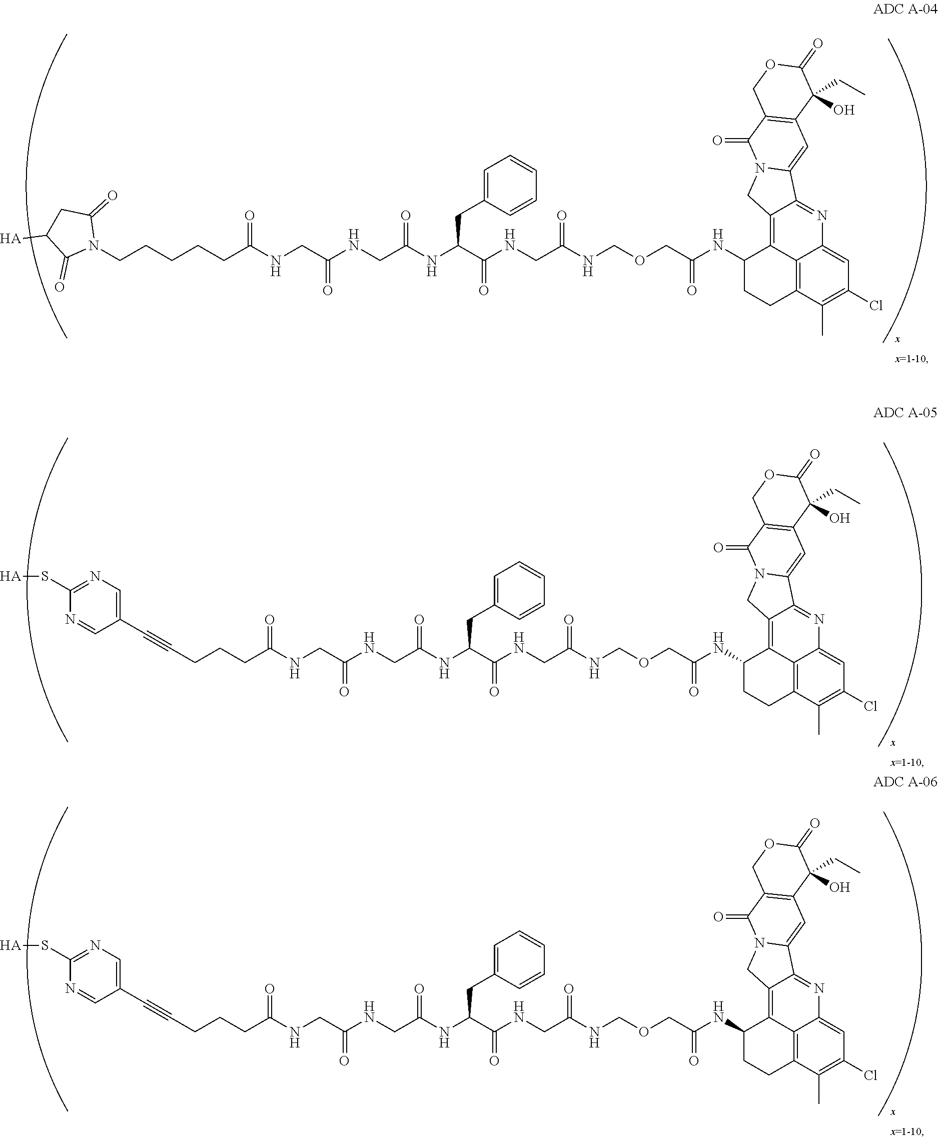 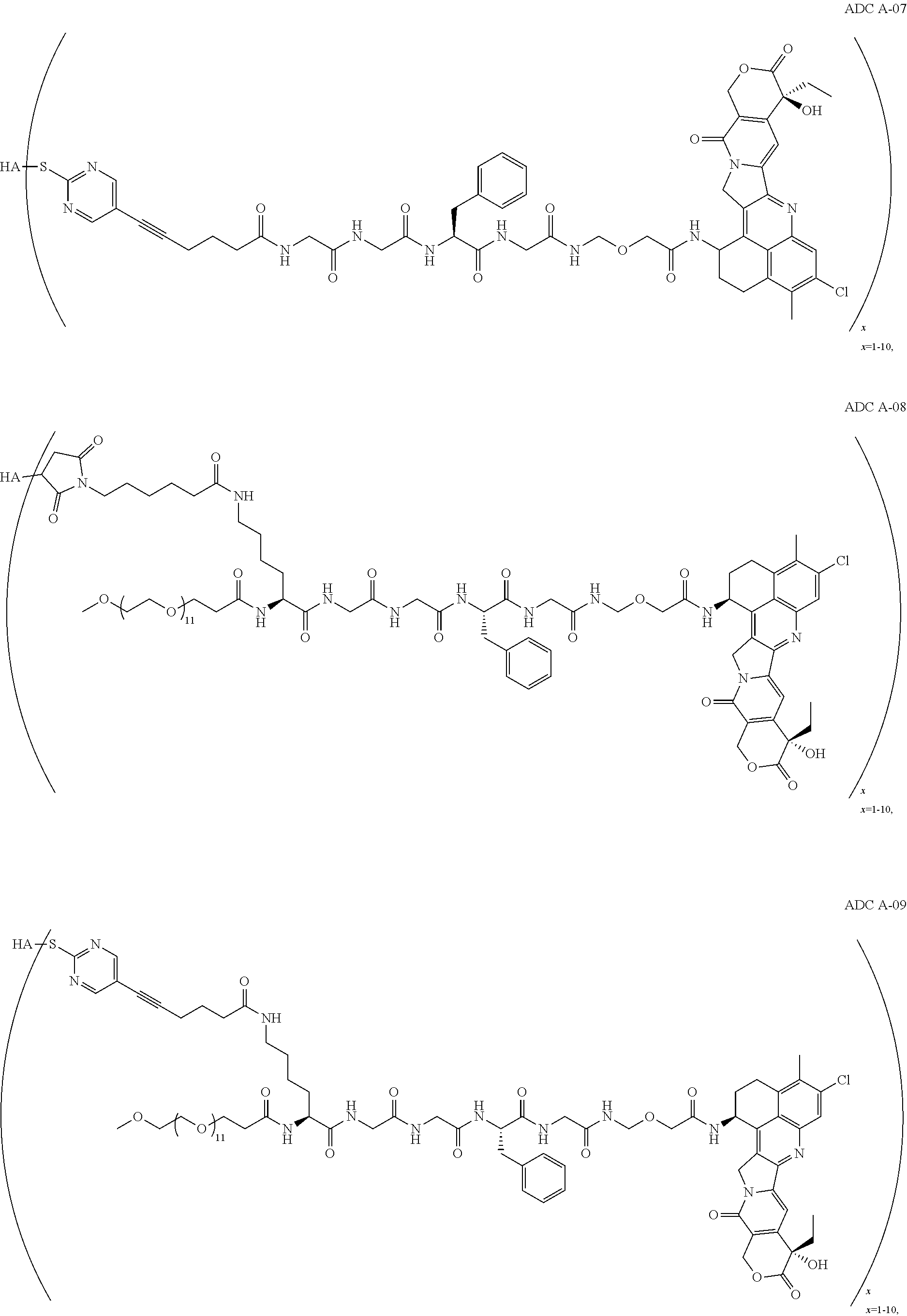 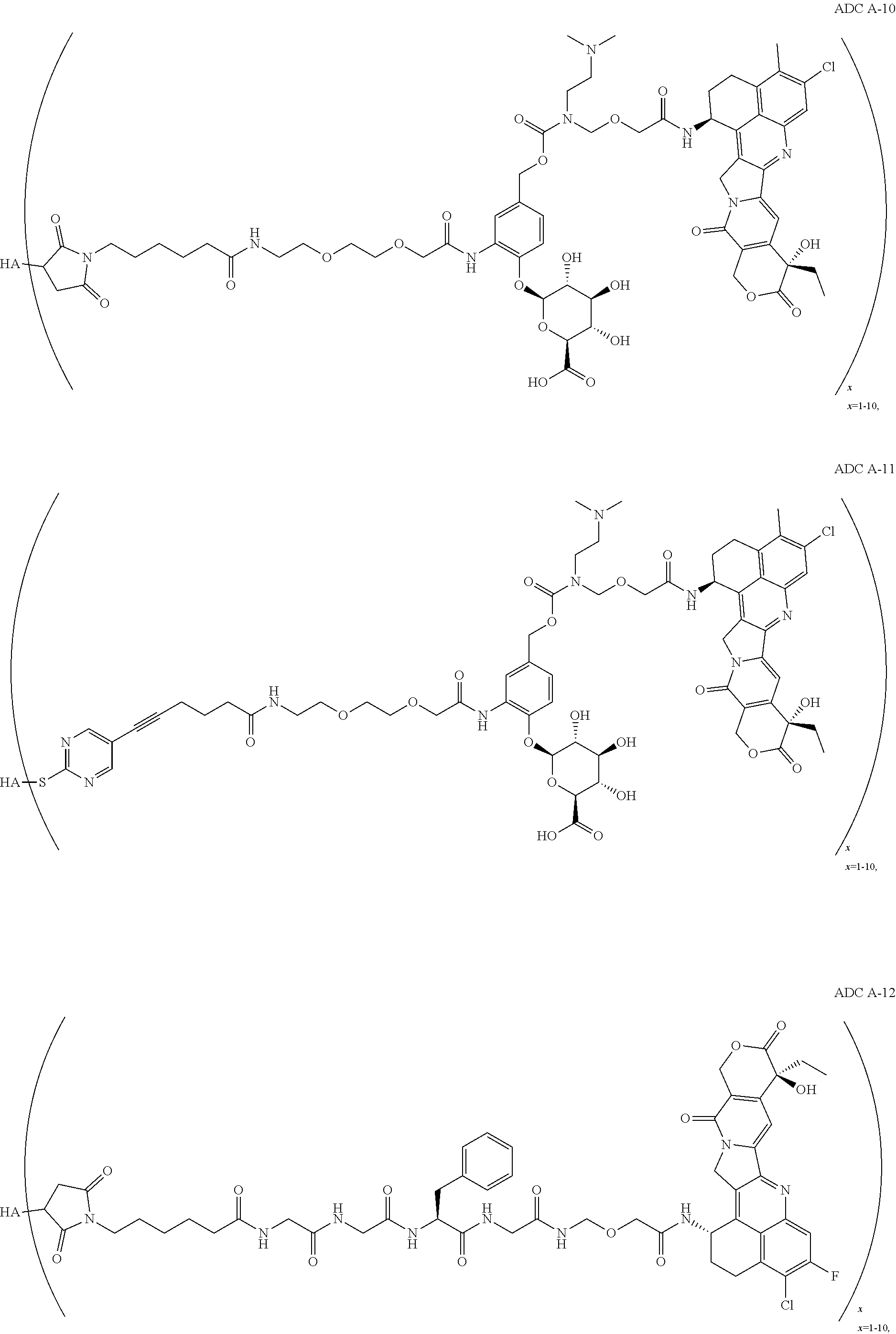 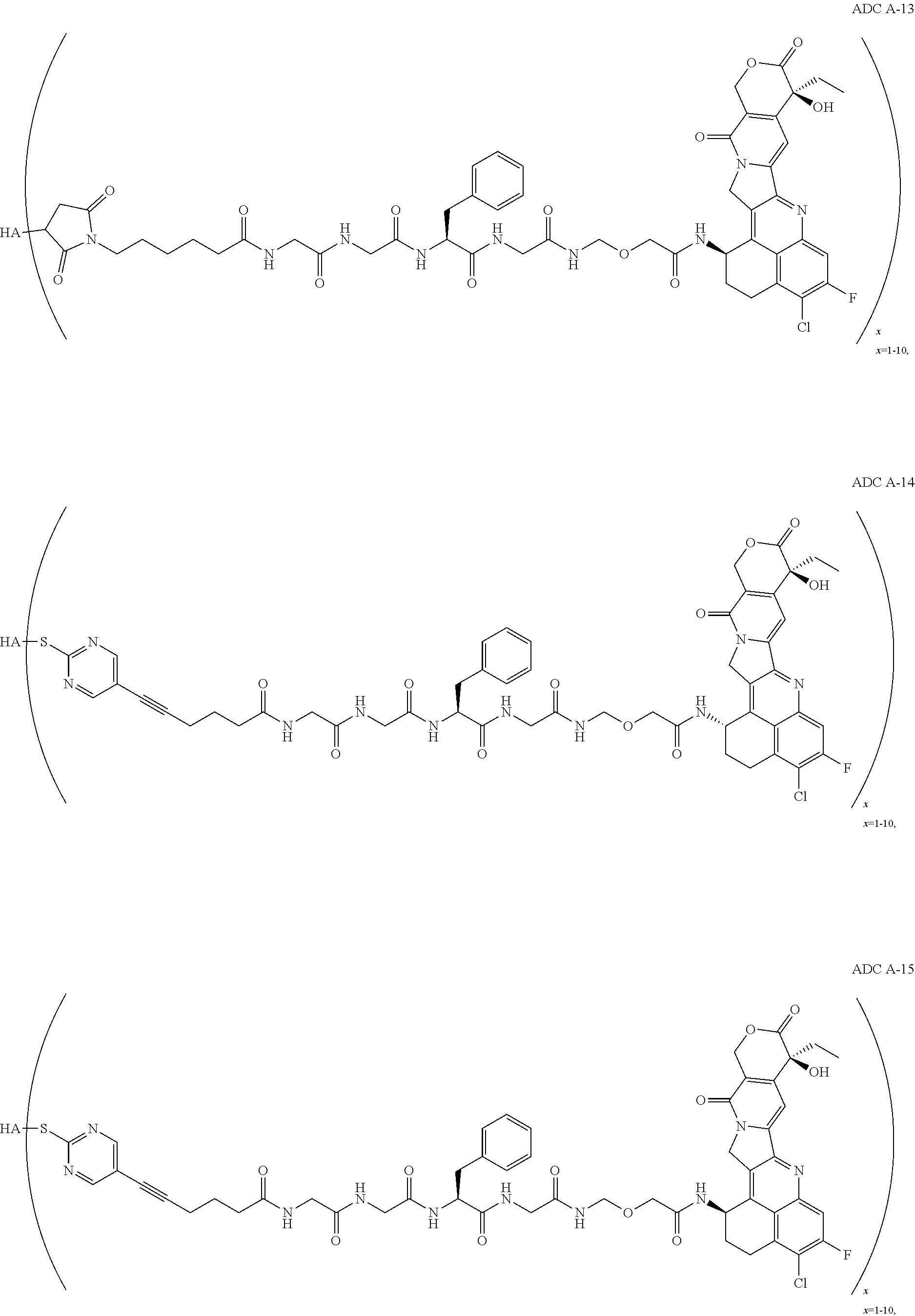 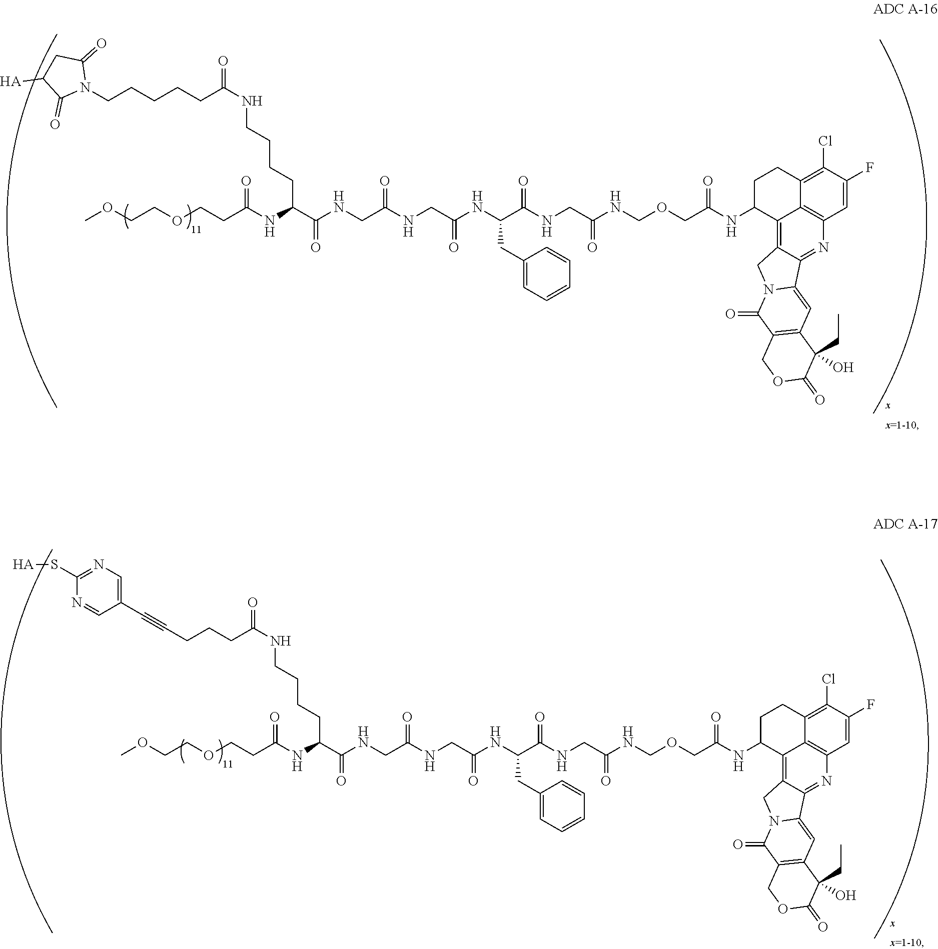 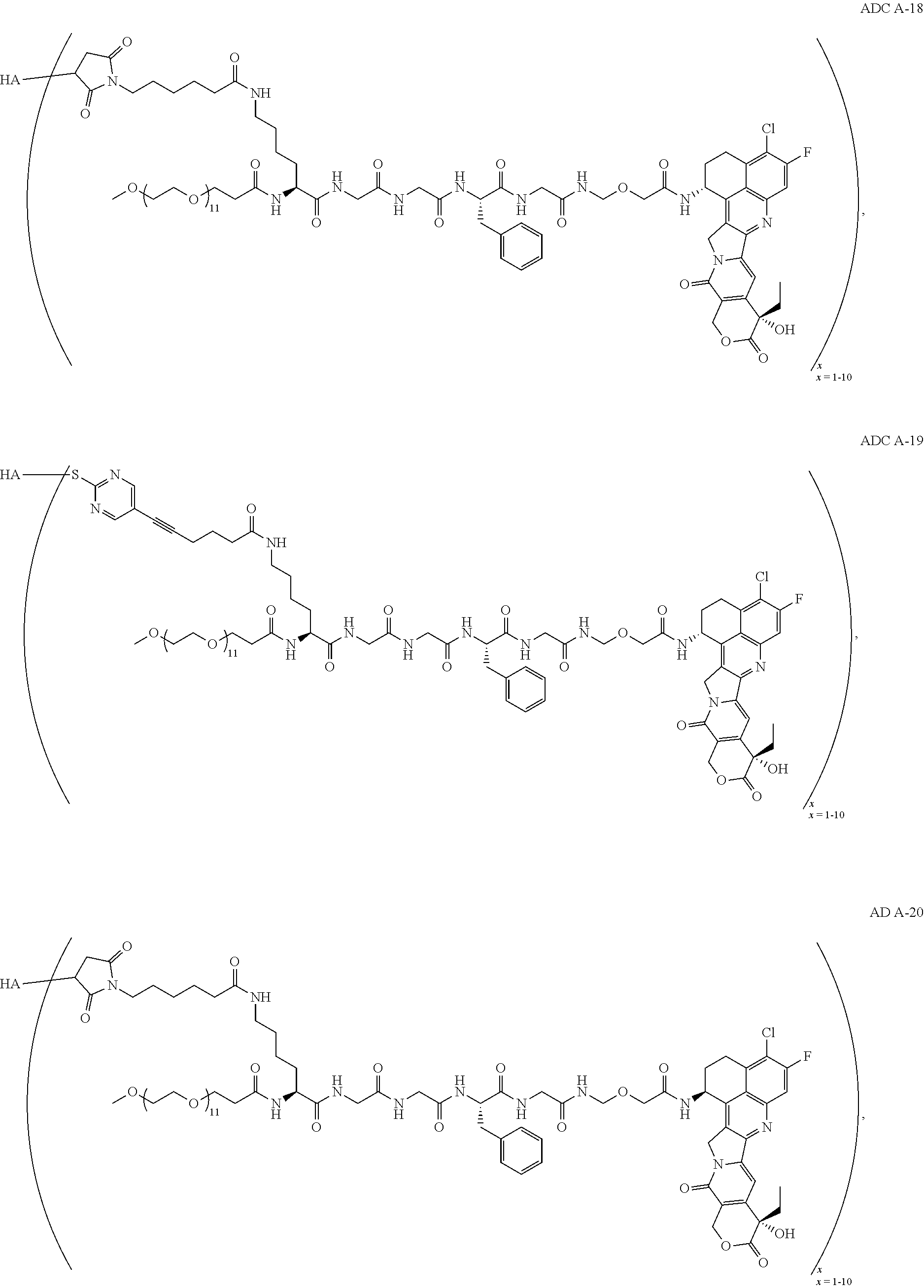 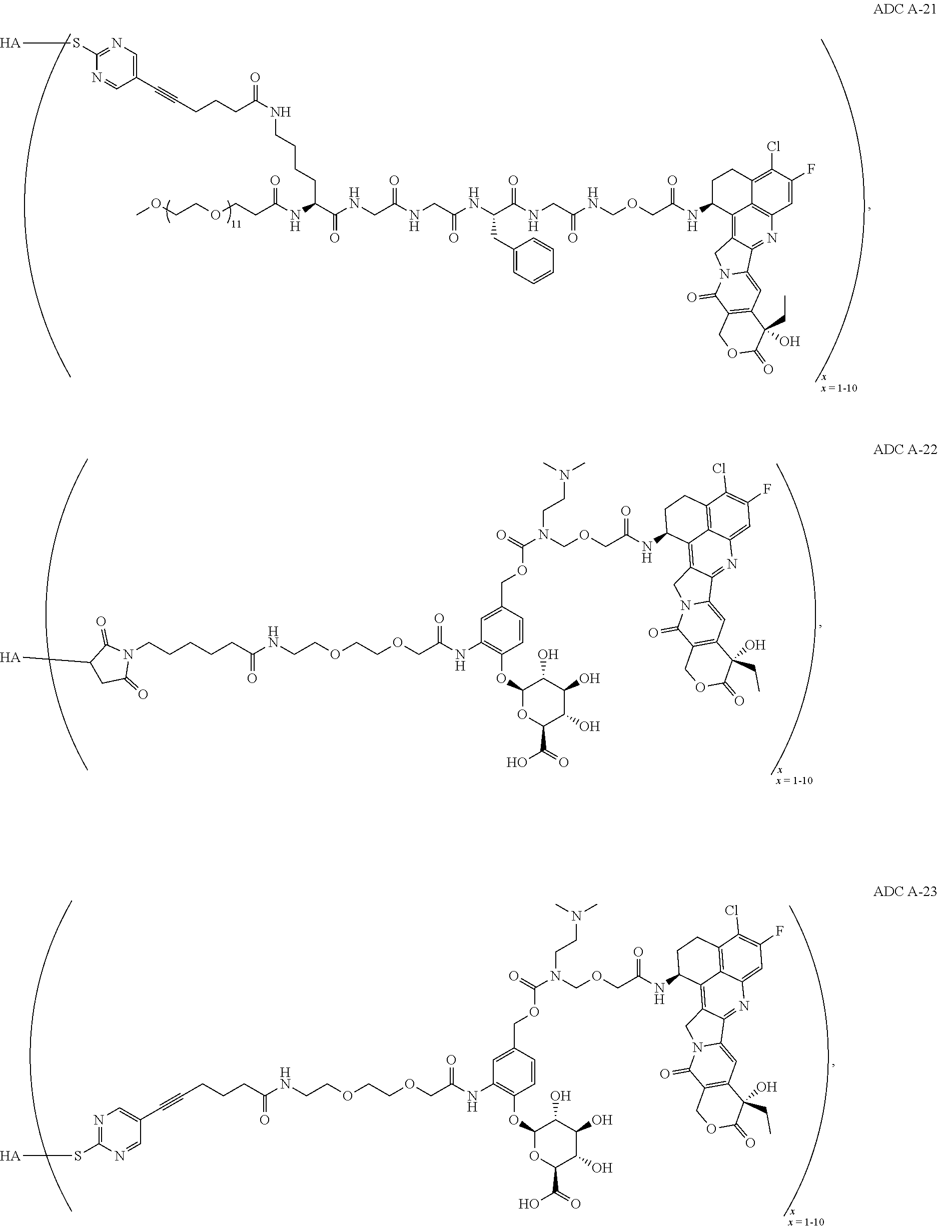 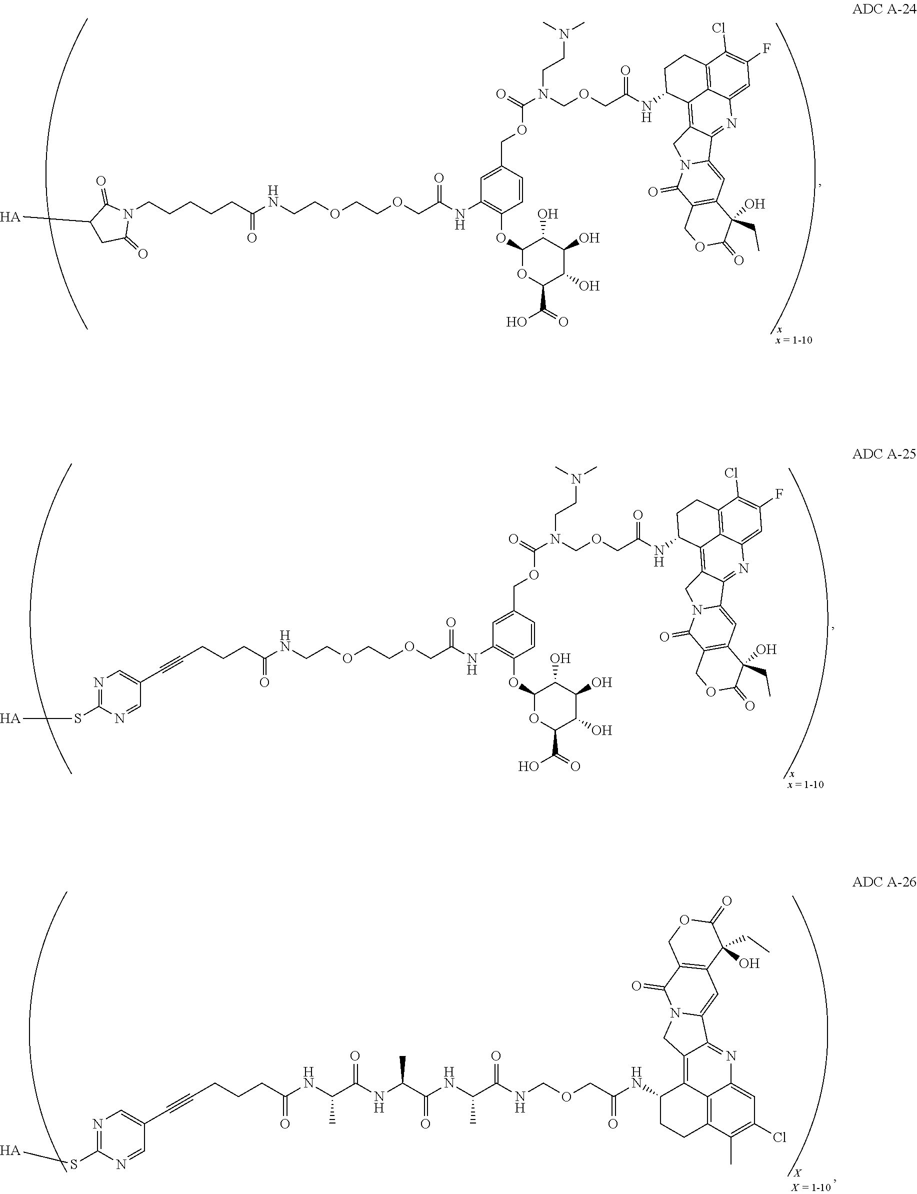 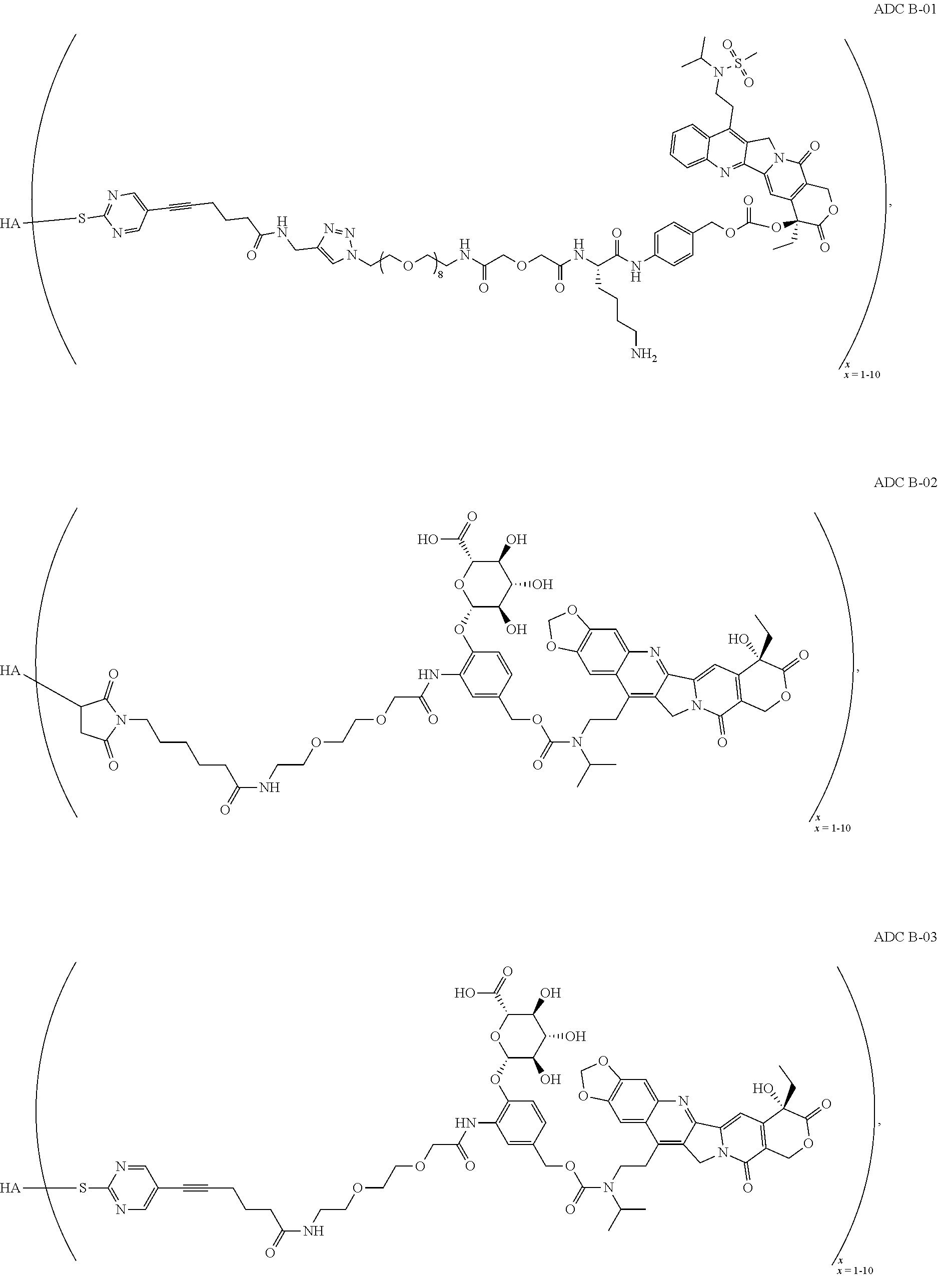 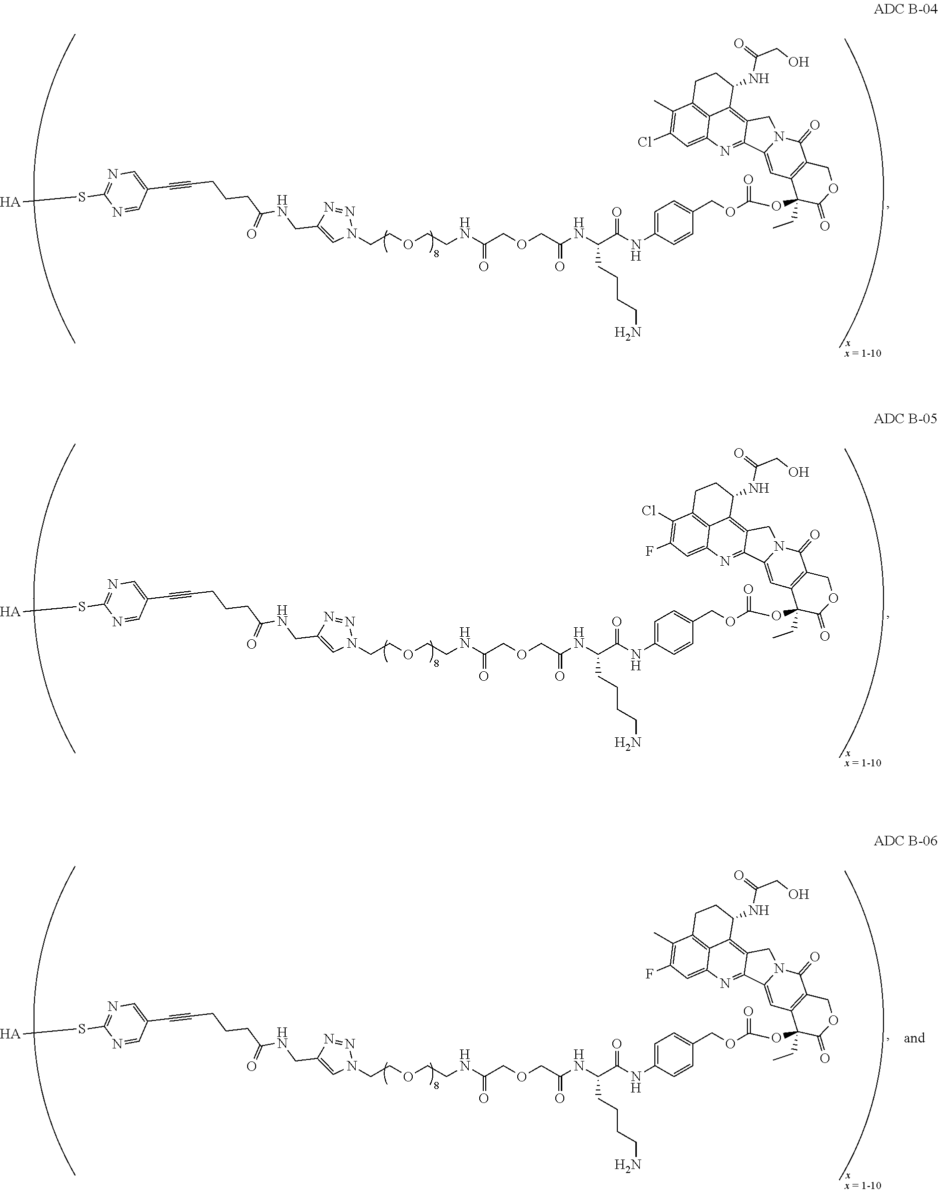 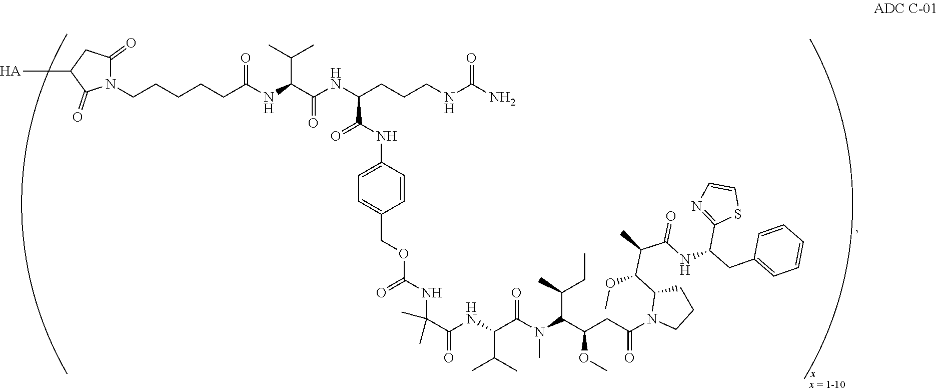
where HA in each antibody-drug conjugate represents antibodies or antigen-binding fragments thereof comprising VH shown as SEQ ID NO: 1, 3, 5, 7 or 9 and VL shown as SEQ ID NO: 2, 4, 6, 8 or 10,
and 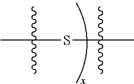 [SMILES: *S*]
represents linkage formed from a sulfhydryl in the antibody or antigen-binding fragment and the linker;
or, the antibody-drug conjugate comprises the formula: 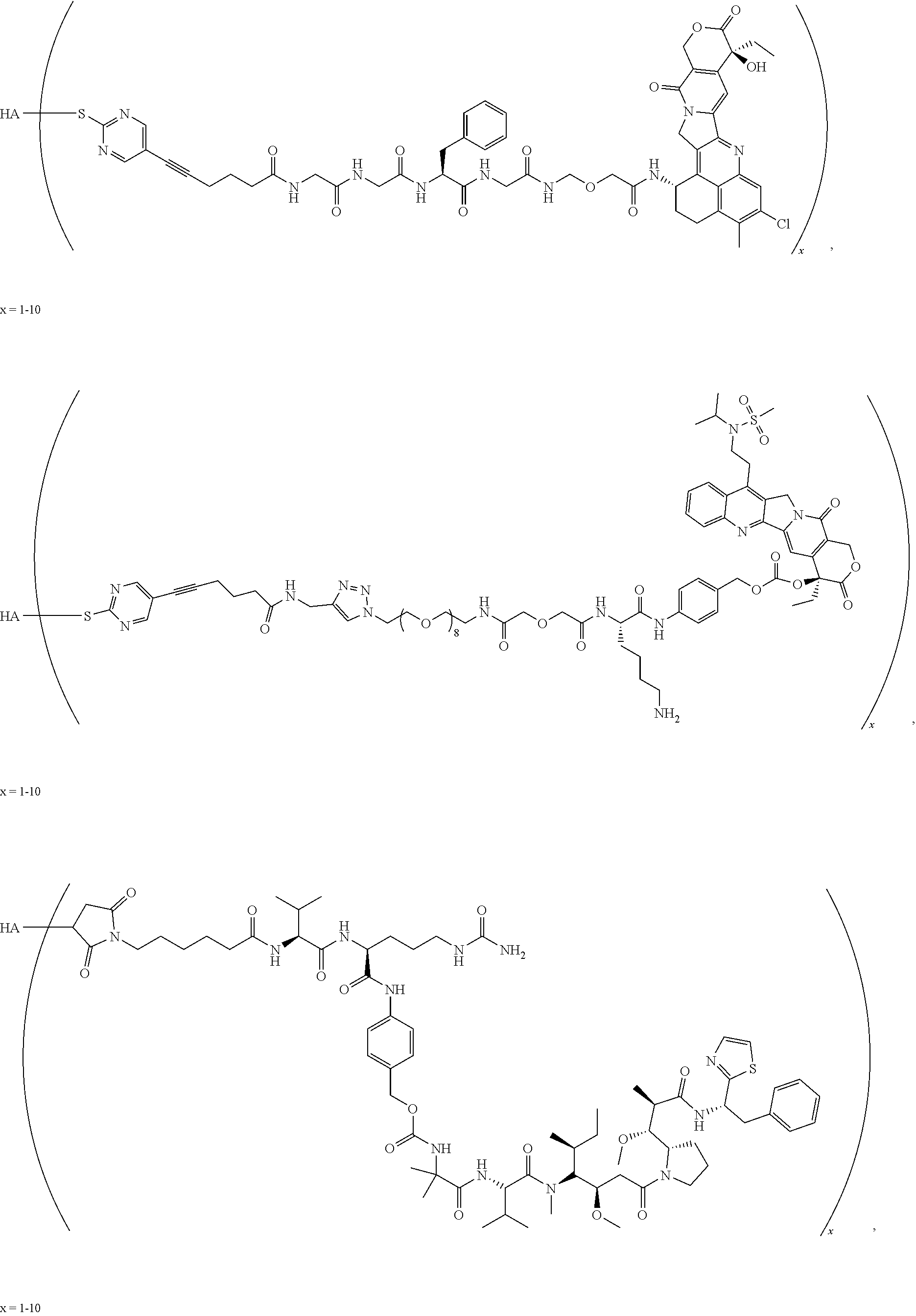 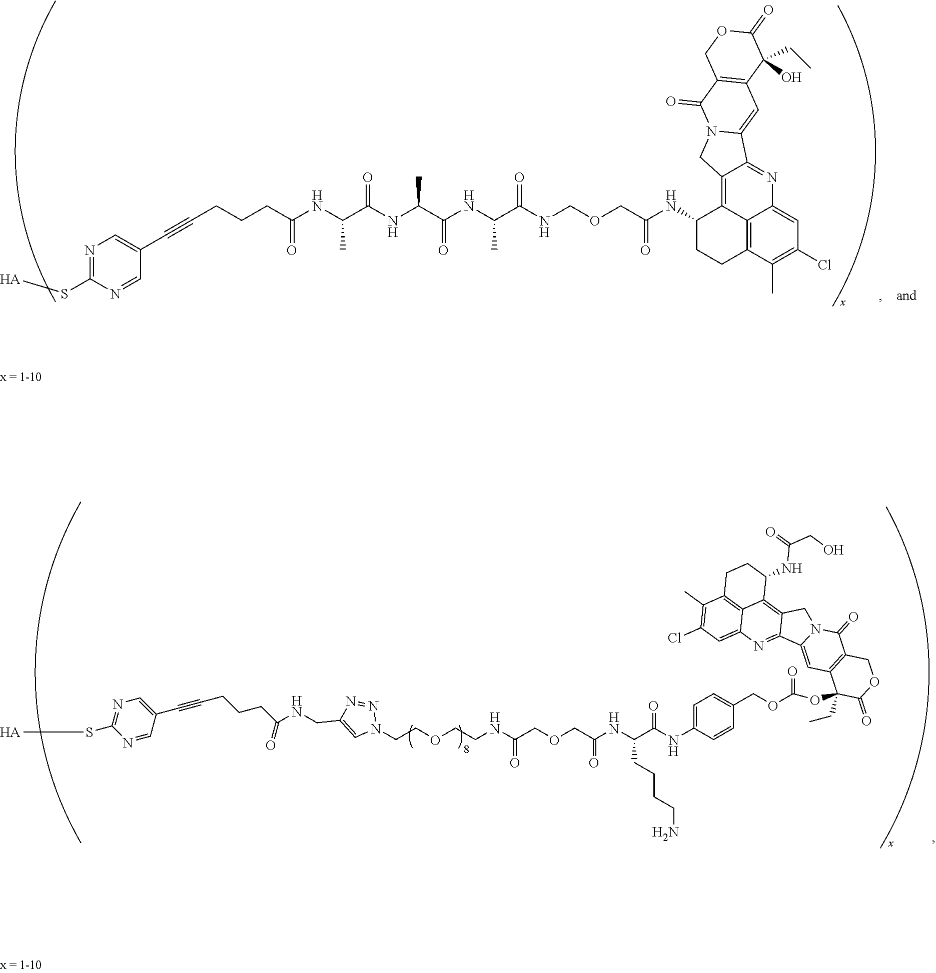
wherein the HA in each antibody-drug conjugate is selected from:
(1) an antibody or antigen-binding fragment thereof comprising a VH having the amino acid sequence as set forth in SEQ ID NO: 1, and a VL having the amino acid sequence as set forth in SEQ ID NO: 2,
(2) an antibody or antigen-binding fragment thereof comprising a VH having the amino acid sequence as set forth in SEQ ID NO: 3, and a VL having the amino acid sequence as set forth in SEQ ID NO: 4,
(3) an antibody or antigen-binding fragment thereof comprising a VH having the amino acid sequence as set forth in SEQ ID NO: 5, and a VL having the amino acid sequence as set forth in SEQ ID NO: 6,
(4) an antibody or antigen-binding fragment thereof comprising a VH having the amino acid sequence as set forth in SEQ ID NO: 7, and a VL having the amino acid sequence as set forth in SEQ ID NO: 8, and
(5) an antibody or antigen-binding fragment thereof comprising a VH having the amino acid sequence as set forth in SEQ ID NO: 9, and a VL having the amino acid sequence as set forth in SEQ ID NO: 10, 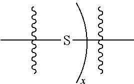 [SMILES: *S*]
represents linkage formed from a sulfhydryl in the antibody or antigen-binding fragment and the linker, wherein
HA is linked via the sulfhydryls in the antibody or antigen-binding fragment to form the antibody-drug conjugate;
or, the antibody-drug conjugate comprises the formula: 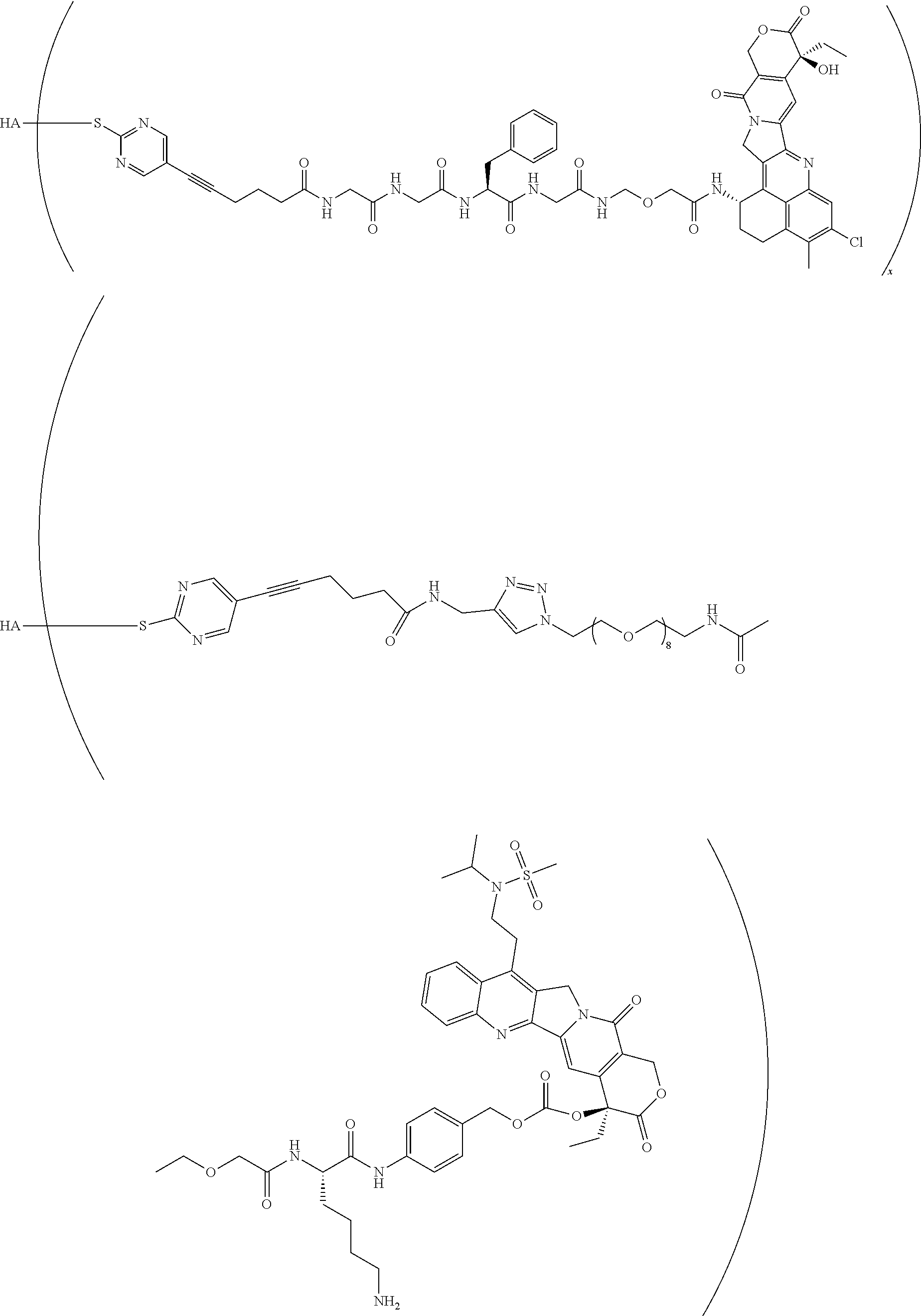 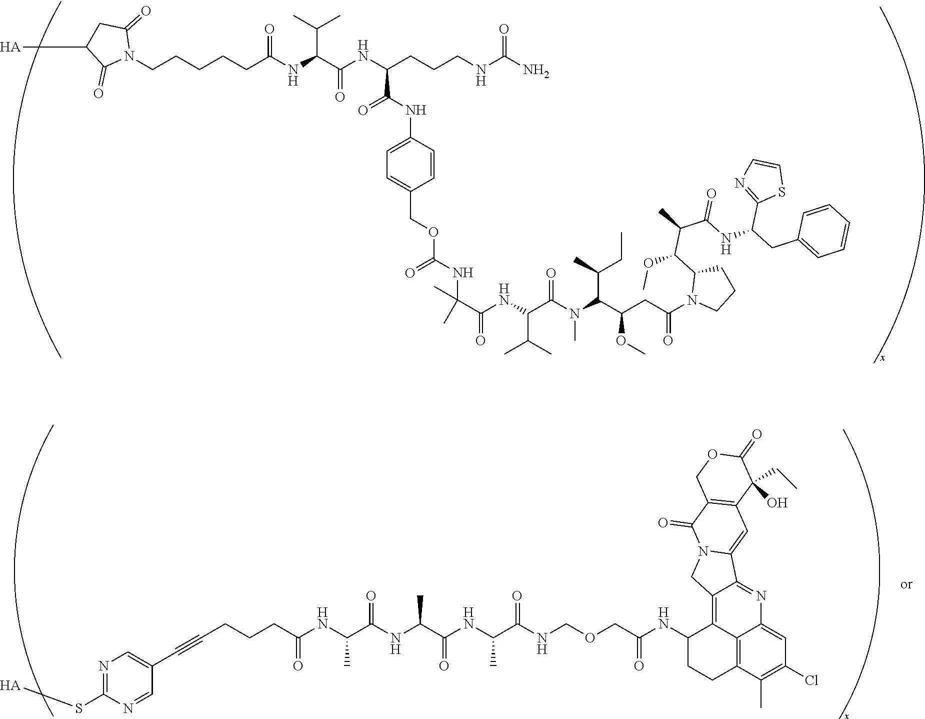 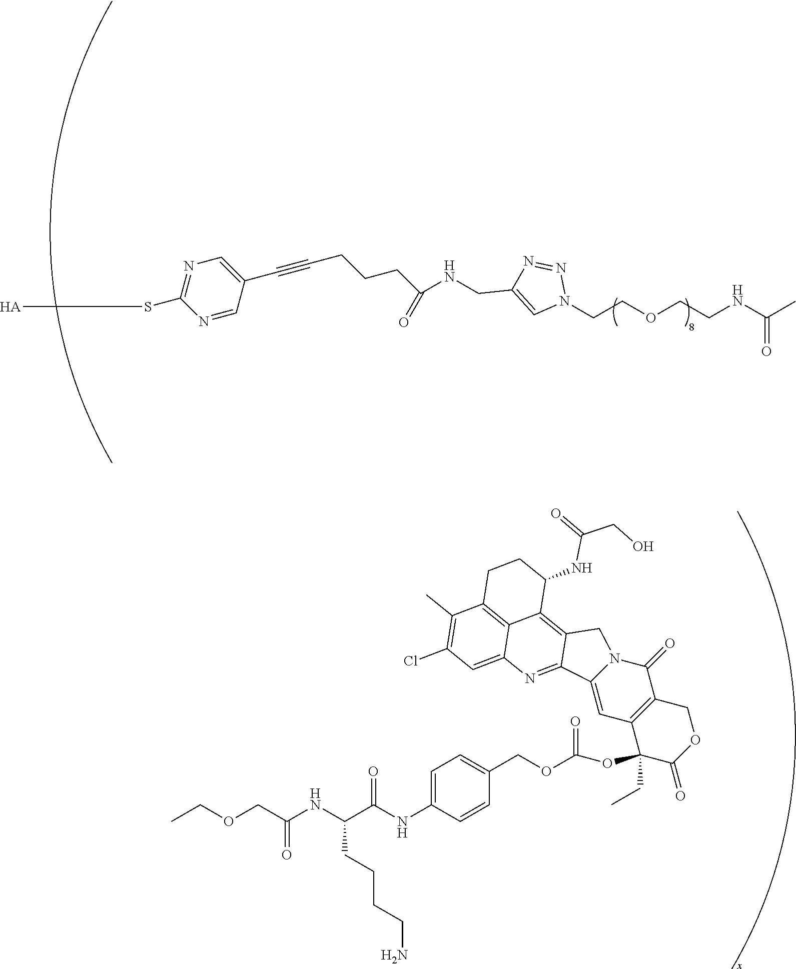
wherein the HA in each antibody-drug conjugate is selected from:
(1) an antibody or antigen-binding fragment thereof comprising a VH having the amino acid sequence as set forth in SEQ ID NO: 1 and a CH having the amino acid sequence as set forth in SEQ ID NO: 43 or 45, and a VL having the amino acid sequence as set forth in SEQ ID NO: 2, and a CL having the amino acid sequence as set forth in SEQ ID NO: 44;
(2) an antibody or antigen-binding fragment thereof comprising a VH having the amino acid sequence as set forth in SEQ ID NO: 3 and a CH having the amino acid sequence as set forth in SEQ ID NO: 43 or 45, and a VL having the amino acid sequence as set forth in SEQ ID NO: 4, and a CL having the amino acid sequence as set forth in SEQ ID NO: 44;
(3) an antibody or antigen-binding fragment thereof comprising a VH having the amino acid sequence as set forth in SEQ ID NO: 5 and a CH having the amino acid sequence as set forth in SEQ ID NO: 43 or 45, and a VL having the amino acid sequence as set forth in SEQ ID NO: 6, and a CL having the amino acid sequence as set forth in SEQ ID NO: 44;
(4) an antibody or antigen-binding fragment thereof comprising a VH having the amino acid sequence as set forth in SEQ ID NO: 7 and a CH having the amino acid sequence as set forth in SEQ ID NO: 43 or 45, and a VL having the amino acid sequence as set forth in SEQ ID NO: 8, and a CL having the amino acid sequence as set forth in SEQ ID NO: 44; and
(5) an antibody or antigen-binding fragment thereof comprising a VH having the amino acid sequence as set forth in SEQ ID NO: 9 and a CH having the amino acid sequence as set forth in SEQ ID NO: 43 or 45, and a VL having the amino acid sequence as set forth in SEQ ID NO: 10, and a CL having the amino acid sequence as set forth in SEQ ID NO: 44; 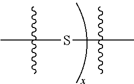 [SMILES: *S*]
represents linkage formed from a sulfhydryl in the antibody or antigen-binding fragment and the linker; wherein
HA is linked via one or more sulfhydryls in the antibody or antigen-binding fragment to form the antibody-drug conjugate;
and x is 3 to 9;
or, the antibody or antigen-binding fragment thereof comprises VH shown as SEQ ID NO: 3 and CH shown as SEQ ID NO: 43 or 45, VL shown as SEQ ID NO: 4 and CL shown as SEQ ID NO: 44.

85. The antibody-drug conjugate according to claim 74, wherein the antibody-drug conjugate is: 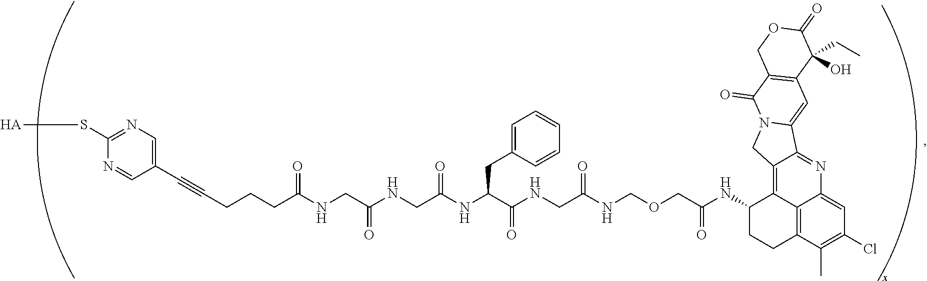
wherein HA in the antibody-drug conjugate is an antibody or antigen-binding fragment thereof comprising: a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 44; 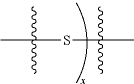 [SMILES: *S*]
represents linkage formed from a sulfhydryl in the antibody or antigen-binding fragment and the linker;
HA is linked via the sulfhydryls in the antibody or antigen-binding fragment to form the antibody-drug conjugate;
and x is 7 to 9;
or, the antibody-drug conjugate is: 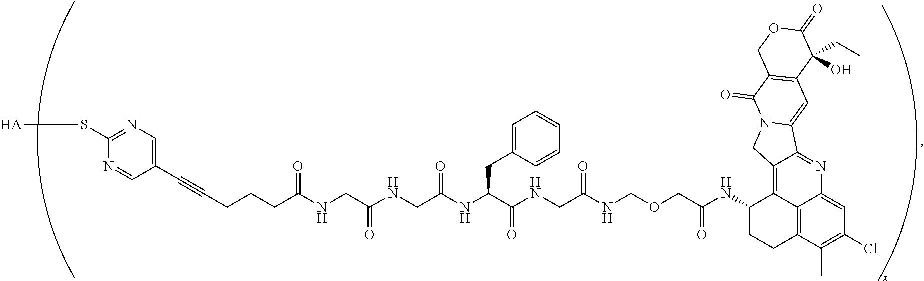
wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; 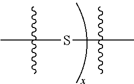 [SMILES: *S*]
represents linkage formed from a sulfhydryl in the antibody or antigen-binding fragment and the linker;
HA is linked via the sulfhydryls of the cysteines in the antibody or antigen-binding fragment to form the antibody-drug conjugate;
and x is 7 to 8;
or, the antibody-drug conjugate is: 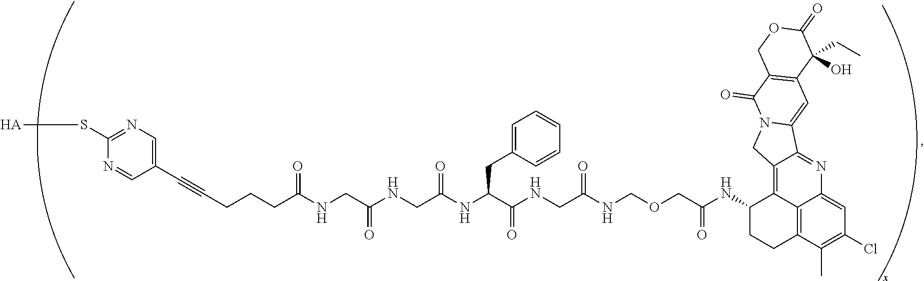
wherein HA is an antibody comprising a heavy chain (HC) consisting of the amino acid sequence as set forth in SEQ ID NO: 52, 53 or 54 and a light chain (LC) consisting of the amino acid sequence as set forth in SEQ ID NO: 47; 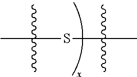 [SMILES: *S*]
represents linkage formed from a sulfhydryl in the antibody or antigen-binding fragment and the linker;
HA is linked via the sulfhydryls of the cysteines in the antibody or antigen-binding fragment to form the antibody-drug conjugate;
and x is 7 to 8;
or, the antibody-drug conjugate is: 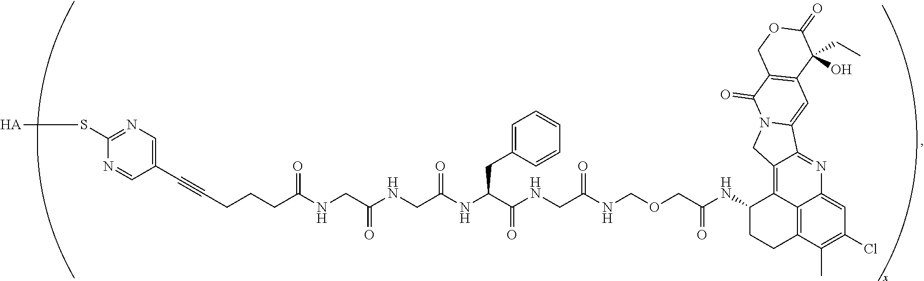
wherein HA is an antibody consisting of two heavy chains and two light chains, each heavy chain (HC) consists of the amino acid sequence as set forth in SEQ ID NO: 52 and each light chain (LC) consists of the amino acid sequence as set forth in SEQ ID NO: 47; and 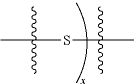 [SMILES: *S*]
represents linkage formed from a sulfhydryl in the antibody or antigen-binding fragment and the linker;
HA is linked via the sulfhydryls of the cysteines in the antibody or antigen-binding fragment to form the antibody-drug conjugate;
and x is 7 to 8;
or, the antibody-drug conjugate is: 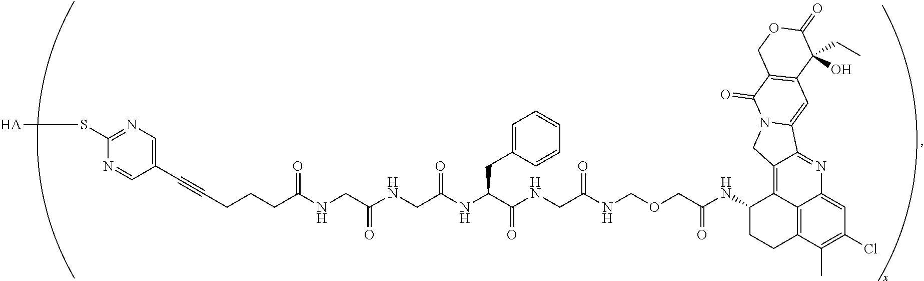
wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; 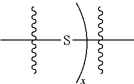 [SMILES: *S*]
represents linkage formed from a sulfhydryl in the antibody or antigen-binding fragment and the linker;
HA is linked via the sulfhydryls of the cysteines in the antibody or antigen-binding fragment to form the antibody-drug conjugate;
and x is 8.

86. The antibody-drug conjugate according to claim 74, wherein the antibody-drug conjugate is: 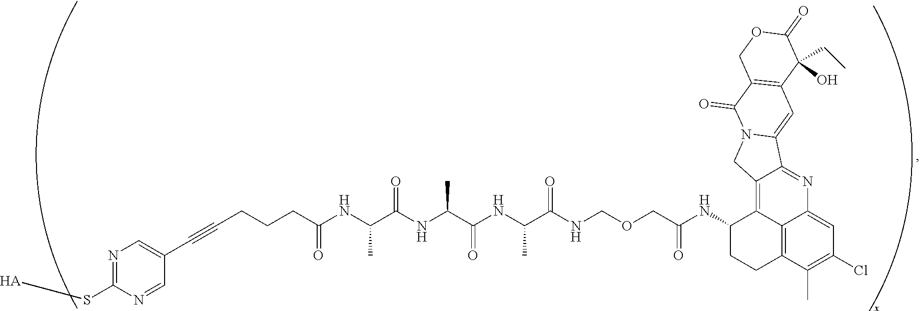
wherein HA in the antibody-drug conjugate is an antibody or antigen-binding fragment thereof comprising: a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 44; 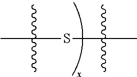 [SMILES: *S*]
represents linkage formed from a sulfhydryl in the antibody or antigen-binding fragment and the linker;
HA is linked via the sulfhydryls in the antibody or antigen-binding fragment to form the antibody-drug conjugate;
and x is 7 to 9;
or, the antibody-drug conjugate is: 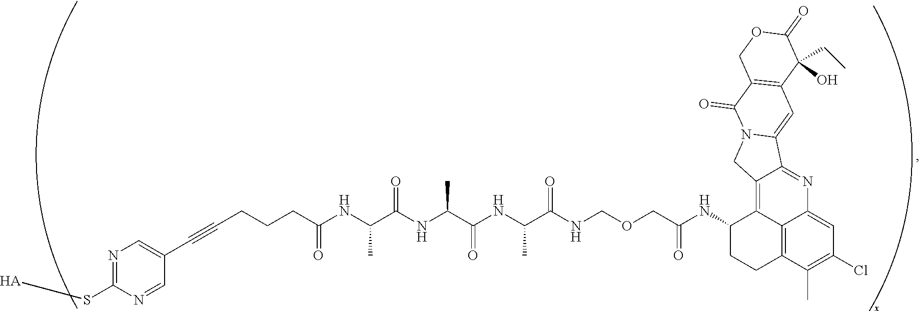
wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; 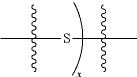 [SMILES: *S*]
represents linkage formed from a sulfhydryl in the antibody or antigen-binding fragment and the linker;
HA is linked via the sulfhydryls in the antibody or antigen-binding fragment to form the antibody-drug conjugate;
and x is 7 to 8;
or, the antibody-drug conjugate is: 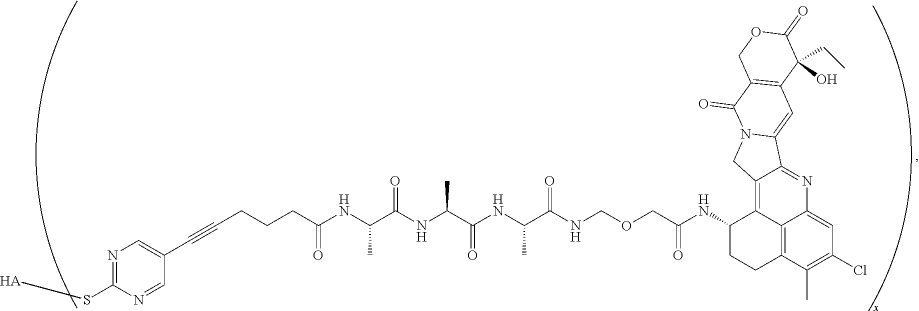
wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; 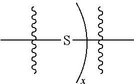 [SMILES: *S*]
represents linkage formed from a sulfhydryl in the antibody or antigen-binding fragment and the linker;
HA is linked via the sulfhydryls in the antibody or antigen-binding fragment to form the antibody-drug conjugate;
and x is 8.

87. The antibody-drug conjugate according to claim 74, wherein the antibody-drug conjugate is: 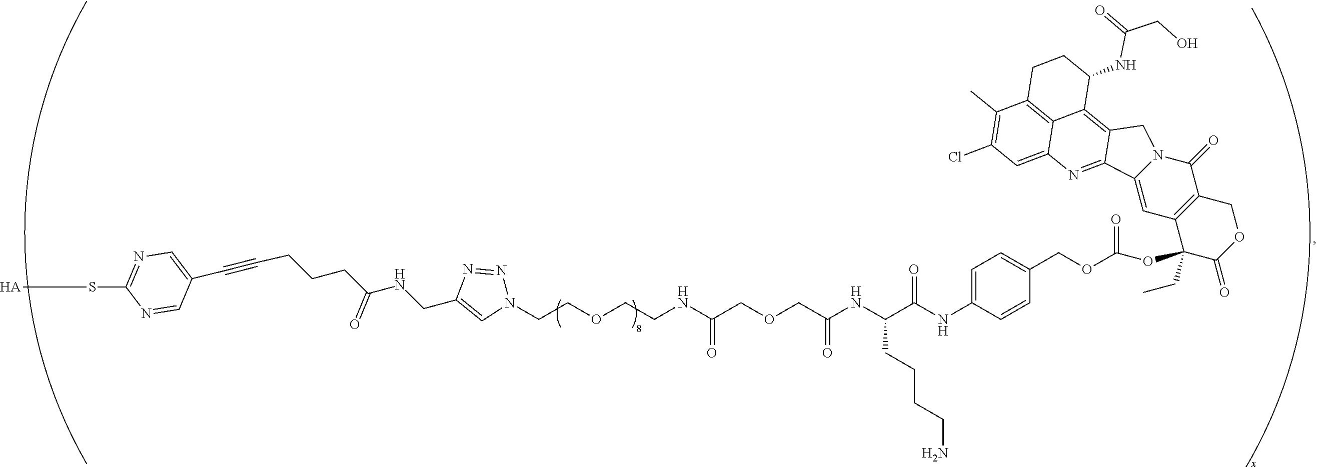
wherein HA in the antibody-drug conjugate is an antibody or antigen-binding fragment thereof comprising: a VH having the amino acid sequence as set forth in SEQ ID NO: 3 and a heavy chain constant region (CH) having the amino acid sequence as set forth in SEQ ID NO: 43 or 45, and a VL having the amino acid sequence as set forth in SEQ ID NO: 4 and a light chain constant region (CL) having the amino acid sequence as set forth in SEQ ID NO: 44; 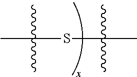 [SMILES: *S*]
represents linkage formed from a sulfhydryl in the antibody or antigen-binding fragment and the linker;
HA is linked via the sulfhydryls in the antibody or antigen-binding fragment to form the antibody-drug conjugate;
and x is 7 to 9;
or, the antibody-drug conjugate is: 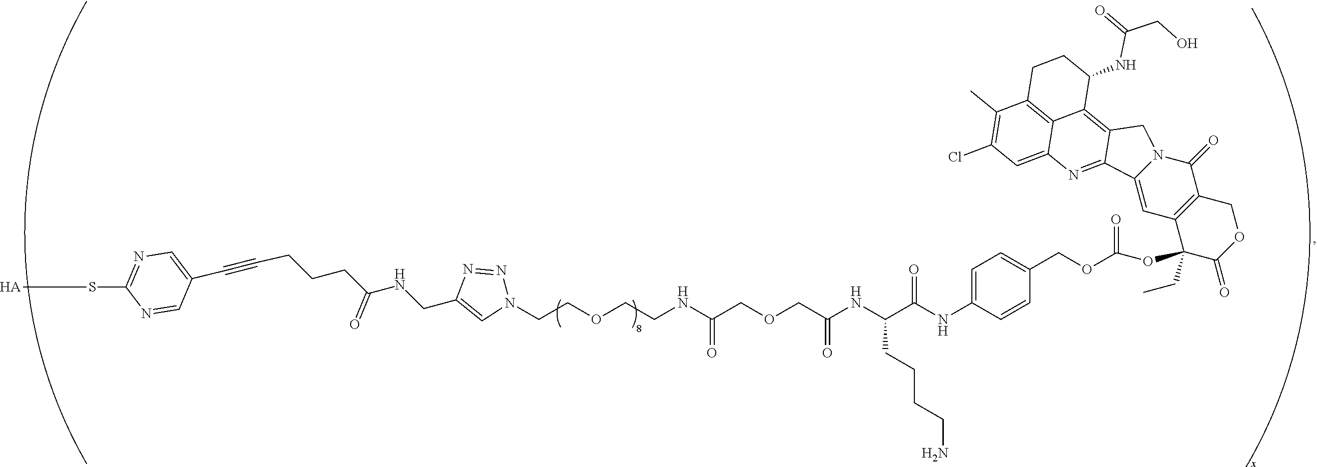
wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; 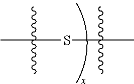 [SMILES: *S*]
represents linkage formed from a sulfhydryl in the antibody or antigen-binding fragment and the linker;
HA is linked via the sulfhydryls in the antibody or antigen-binding fragment to form the antibody-drug conjugate;
and x is 7 to 8;
or, the antibody-drug conjugate is: 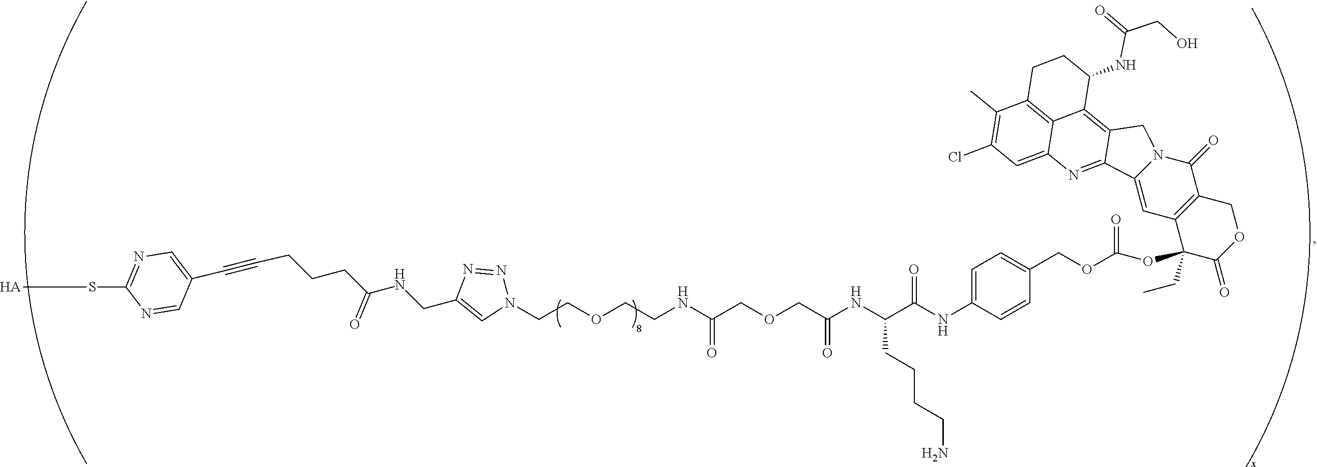
wherein HA is an antibody or antigen-binding fragment thereof comprising a heavy chain (HC) having the amino acid sequence as set forth in SEQ ID NO: 46 and a light chain (LC) having the amino acid sequence as set forth in SEQ ID NO: 47; 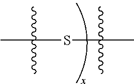 [SMILES: *S*]
represents linkage formed from a sulfhydryl in the antibody or antigen-binding fragment and the linker;
HA is linked via the sulfhydryls in the antibody or antigen-binding fragment to form the antibody-drug conjugate;
and x is 8.

88. A composition comprising one or more antibody-drug conjugate according to claim 74, wherein the DAR (drug-antibody conjugation ratio) of the composition is 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4-7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10.

89. The antibody-drug conjugate according to claim 74, wherein (i) heavy chain C-terminus of the antibody or antigen-binding fragment thereof lacks a lysine residue; (ii) heavy chain N-terminus of the antibody or antigen-binding fragment thereof is glutamine, glutamic acid, pyroglutamate or pyroglutamic acid; or, (iii) the heavy chain C-terminus lacks a lysine residue and the heavy chain N-terminus is glutamine, glutamic acid, pyroglutamate or pyroglutamic acid;
or, the antibody or antigen binding fragment comprises a heavy chain comprising a VH having the amino acid sequence selected from the group consisting of SEQ ID NOs: 1, 3, 5, 7 and 9, wherein the N-terminal glutamine or glutamic acid of the antibody variable region has undergone cyclization to pyroglutamate or pyroglutamic acid; and/or
the antibody or antigen binding fragment comprises a light chain comprising a VL having the amino acid sequence set forth in SEQ ID NO: 8, wherein the N-terminal glutamic acid of the antibody light chain variable region has undergone cyclization to pyroglutamate or pyroglutamic acid;
or, the antibody or antigen binding fragment comprises:
(a) a heavy chain consisting of the sequence shown as SEQ ID NO: 52 and a light chain consisting of the sequence shown as SEQ ID NO: 47;
(b) a heavy chain consisting of the sequence shown as SEQ ID NO: 53 and a light chain consisting of the sequence shown as SEQ ID NO: 47; or
(c) a heavy chain consisting of the sequence shown as SEQ ID NO: 54 and a light chain consisting of the sequence shown as SEQ ID NO: 47.

90. The antibody-drug conjugate according to claim 74, wherein the antibody or antigen binding fragment is obtainable by expressing the nucleic acid molecules encoding the heavy chains and the light chains of the antibody or antigen-binding fragment thereof in a host cell, wherein the antibody or antigen-binding fragment thereof comprises:
(1) a heavy chain comprising VH sequence shown as SEQ ID NO: 1 and heavy chain constant region (CH) shown as SEQ ID NO: 43, and a light chain comprising VL sequence shown as SEQ ID NO: 2 and light chain constant region (CL) shown as SEQ ID NO: 44;
(2) a heavy chain comprising VH sequence shown as SEQ ID NO: 3 and heavy chain constant region (CH) shown as SEQ ID NO: 43 or 45, and a light chain comprising VL sequence shown as SEQ ID NO: 4 and light chain constant region (CL) shown as SEQ ID NO: 44;
(3) a heavy chain comprising VH sequence shown as SEQ ID NO: 5 and heavy chain constant region (CH) shown as SEQ ID NO: 43, and a light chain comprising VL sequence shown as SEQ ID NO: 6 and light chain constant region (CL) shown as SEQ ID NO: 44;
(4) a heavy chain comprising VH sequence shown as SEQ ID NO: 7 and heavy chain constant region (CH) shown as SEQ ID NO: 43, and a light chain comprising VL sequence shown as SEQ ID NO: 8 and light chain constant region (CL) shown as SEQ ID NO: 44;
(5) a heavy chain comprising VH sequence shown as SEQ ID NO: 9 and heavy chain constant region (CH) shown as SEQ ID NO: 43, and a light chain comprising VL sequence shown as SEQ ID NO: 10 and light chain constant region (CL) shown as SEQ ID NO: 44;
(6) a heavy chain (HC) having the amino acid sequence as set forth in SEQ ID NO: 46 and a light chain (LC) having the amino acid sequence as set forth in SEQ ID NO: 47;
(7) a VH having the amino acid sequence set forth in SEQ ID NO: 1 and a VL having the amino acid sequence set forth in SEQ ID NO: 2;
(8) a VH having the amino acid sequence set forth in SEQ ID NO: 3 and a VL having the amino acid sequence set forth in SEQ ID NO: 4;
(9) a VH having the amino acid sequence set forth in SEQ ID NO: 5 and a VL having the amino acid sequence set forth in SEQ ID NO: 6;
(10) a VH having the amino acid sequence set forth in SEQ ID NO: 7 and a VL having the amino acid sequence set forth in SEQ ID NO: 8; or
(11) a VH having the amino acid sequence set forth in SEQ ID NO: 9 and a VL having the amino acid sequence set forth in SEQ ID NO: 10.

91. An antibody-drug conjugate comprising an antibody that binds PTK7 conjugated via one or more cysteine or lysine residue or non-canonical amino acid substitutions of amino acids of the antibody to a drug linker having a structure shown as the formula M-L-E-D, wherein
M is 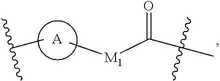
wherein ring A is a 5-6 membered aliphatic heterocyclic ring, or 5-20 membered aromatic ring system, and the aliphatic heterocyclic ring and aromatic ring systems are optionally substituted by one or more members selected from the group consisting of oxo (═O), halogen, cyano, amino, carboxyl, mercapto and C₁₋₆ alkyl;
M₁ is selected from a single bond, C₁₋₂₀ alkylene, C₂₋₂₀ alkenylene and C₂₋₂₀ alkynylene;
L is a linker between M and E;
E is a structural fragment linking L and D; and
D is a cytotoxic drug fragment;
or, the antibody-drug conjugate is characterized by any of the following items (I) to (III): 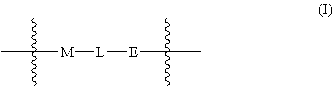
is selected from 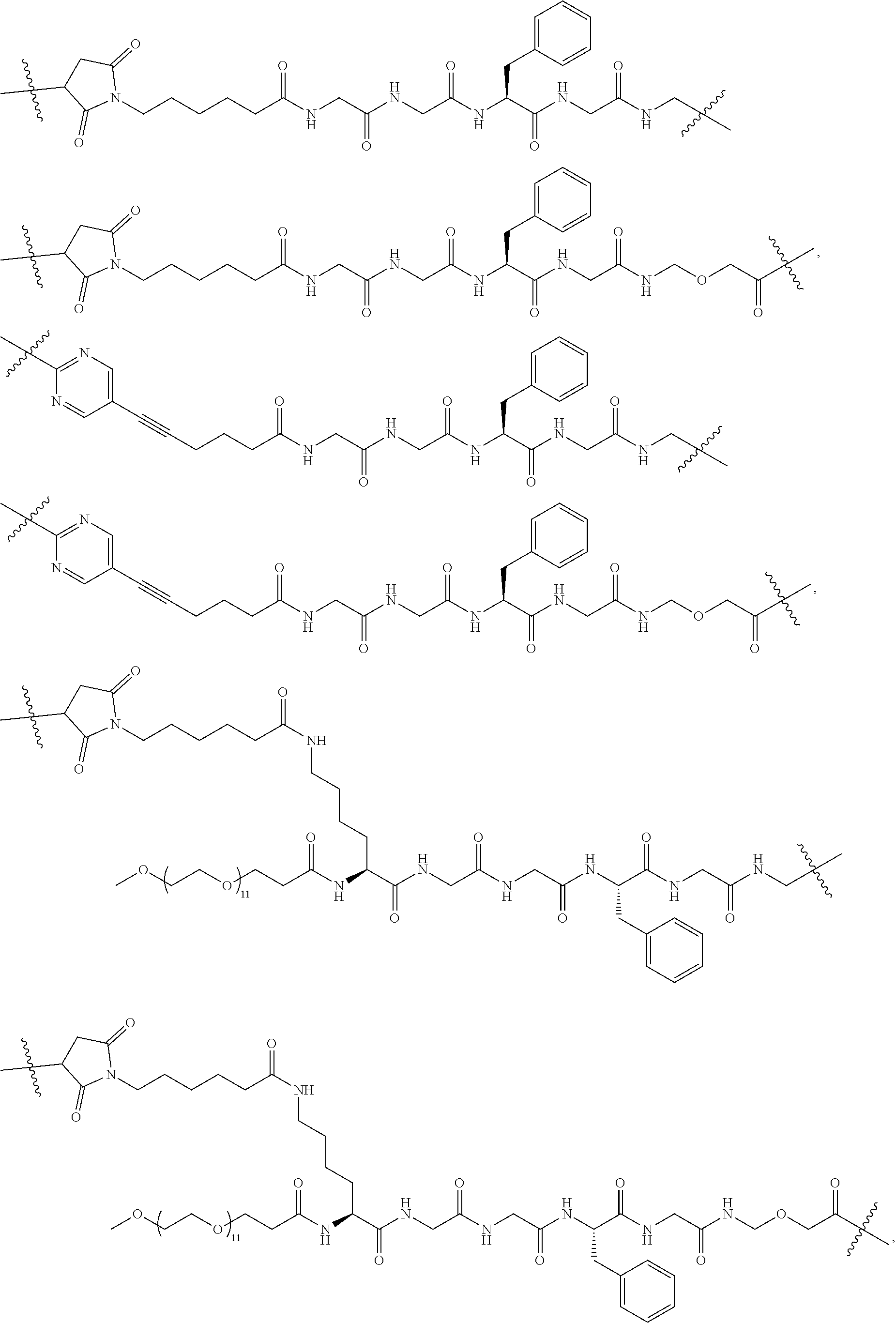 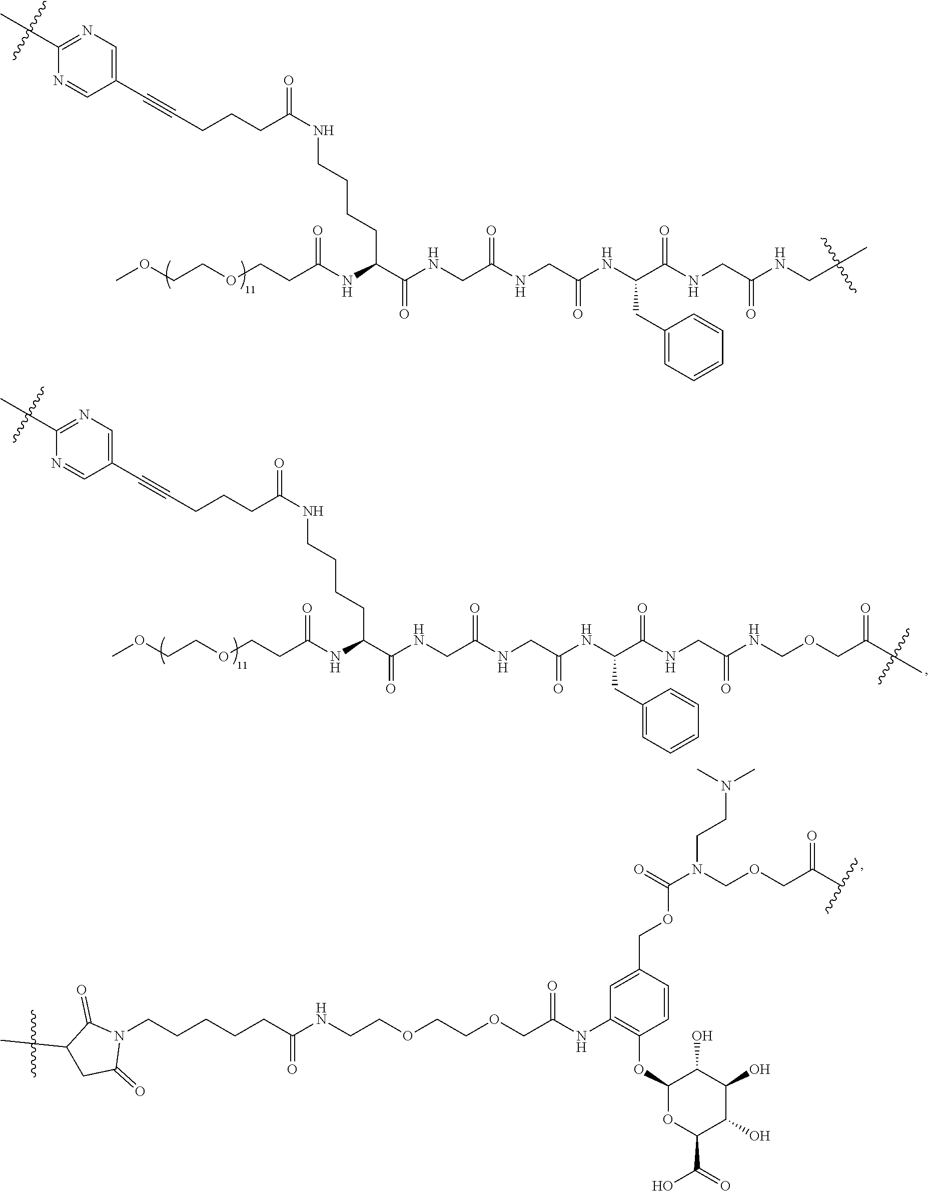 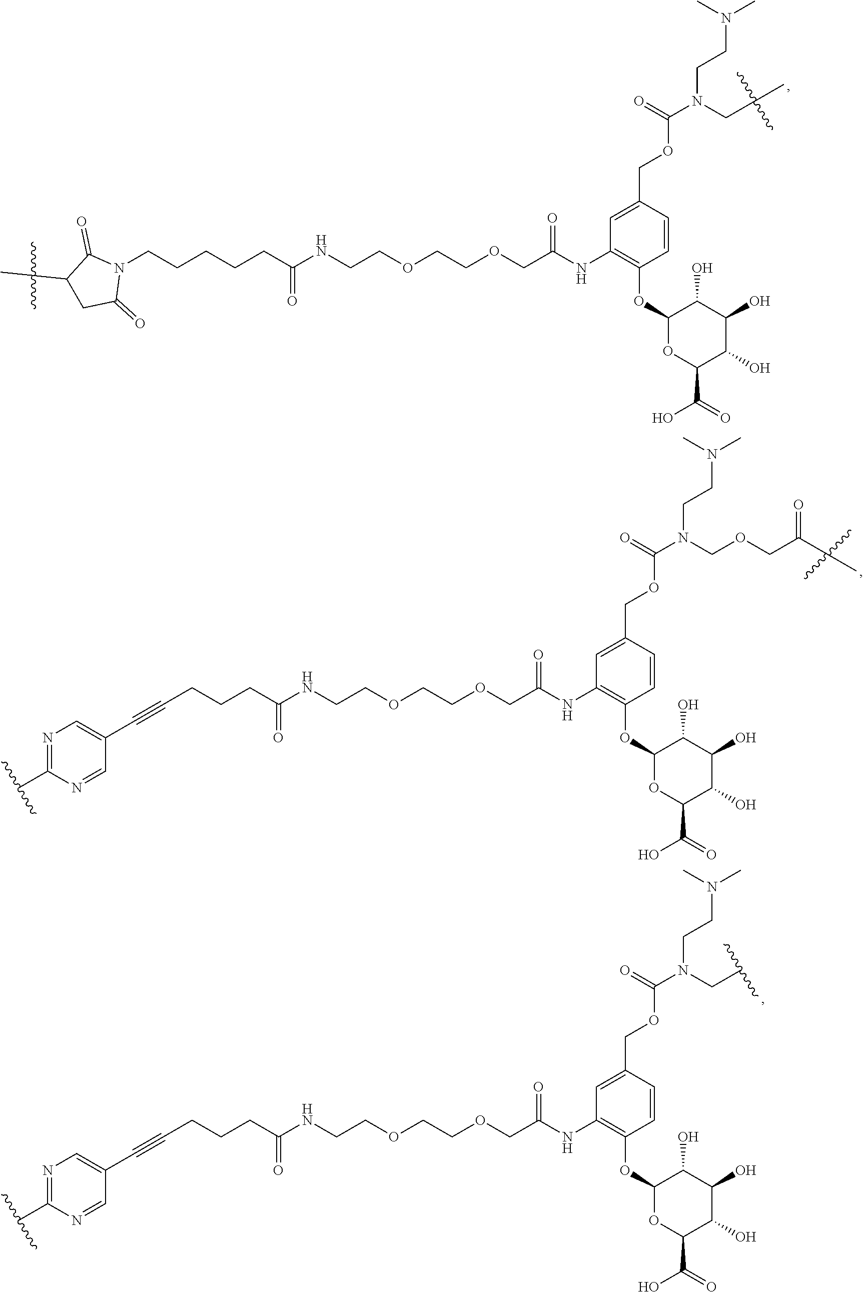 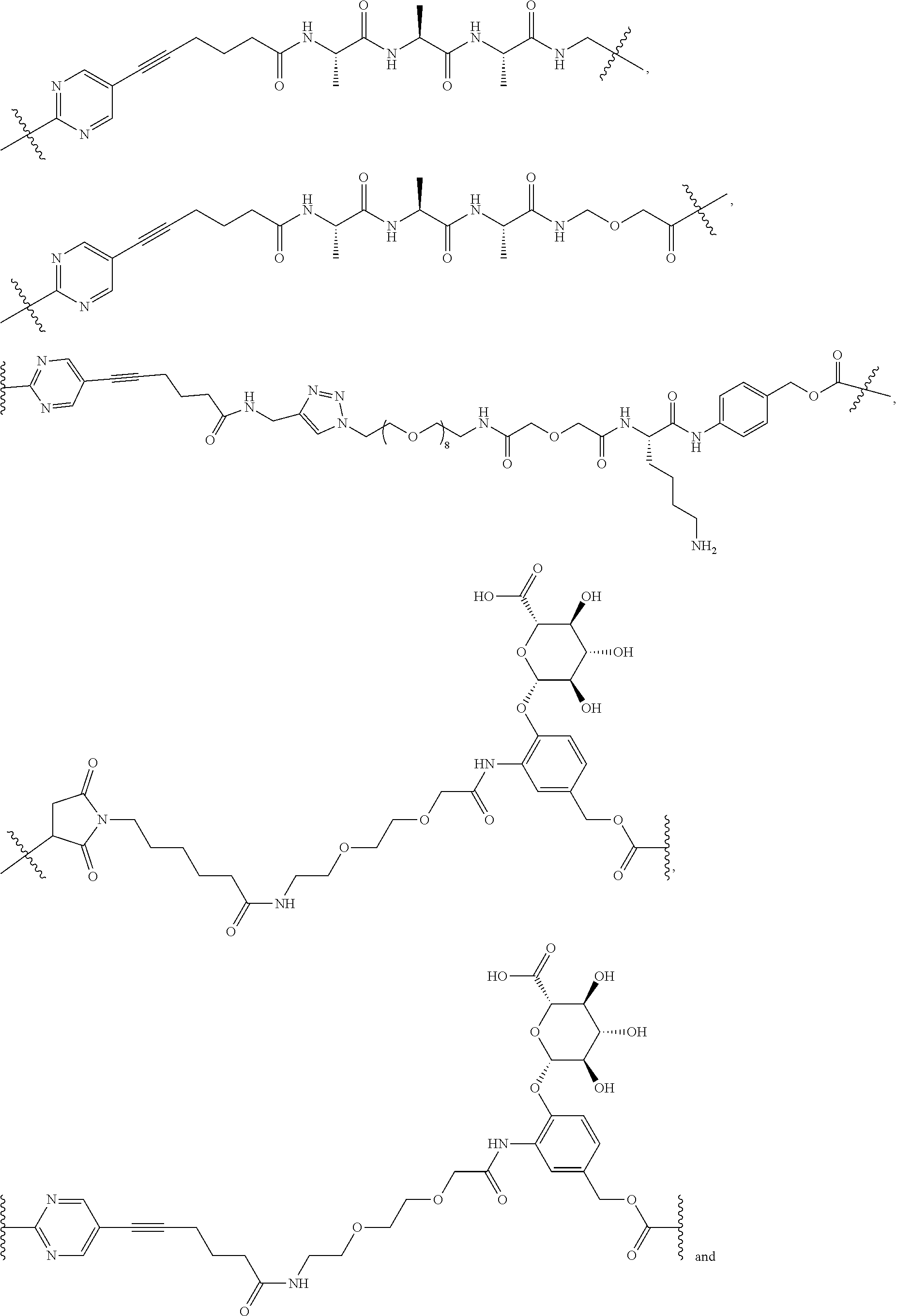 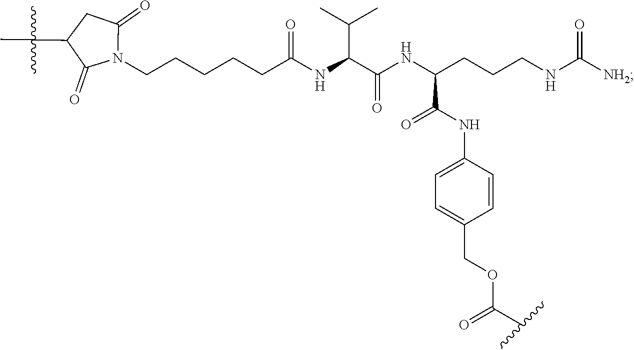 [SMILES: *C(=O)OCc1ccc(NC(=O)[C@H](CCCNC(N)=O)NC(=O)[C@@H](NC(=O)CCCCCN2C(=O)CC(*)C2=O)C(C)C)cc1]
(II) the cytotoxic drug is selected from the following compounds: 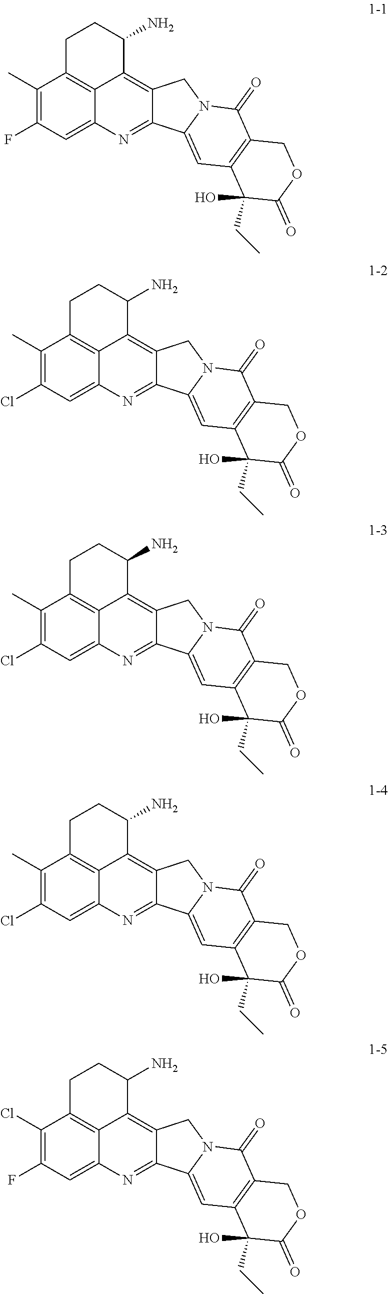 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(Cl)c(C)c3c1c2C(N)CC3 ; CC[C@@]1(O)C(=O)OCc2c1cc1n(c2=O)Cc2c-1nc1cc(Cl)c(C)c3c1c2[C@H](N)CC3 ; CC[C@@]1(O)C(=O)OCc2c1cc1n(c2=O)Cc2c-1nc1cc(Cl)c(C)c3c1c2[C@@H](N)CC3 ; CC[C@@]1(O)C(=O)OCc2c1cc1n(c2=O)Cc2c-1nc1cc(F)c(Cl)c3c1c2C(N)CC3] 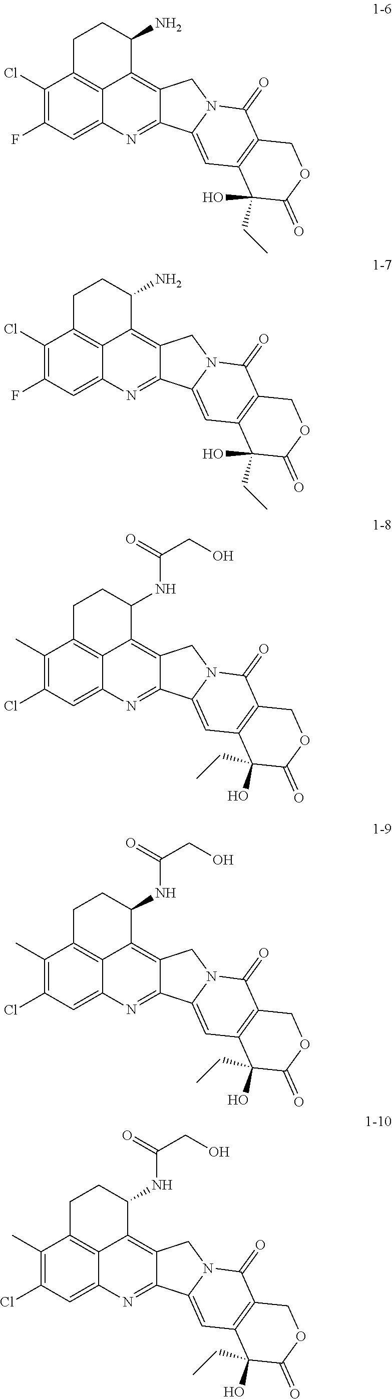 [SMILES: CC[C@@]1(O)C(=O)OCc2c1cc1n(c2=O)Cc2c-1nc1cc(F)c(Cl)c3c1c2[C@H](N)CC3 ; CC[C@@]1(O)C(=O)OCc2c1cc1n(c2=O)Cc2c-1nc1cc(F)c(Cl)c3c1c2[C@@H](N)CC3 ; CC[C@@]1(O)C(=O)OCc2c1cc1n(c2=O)Cc2c-1nc1cc(Cl)c(C)c3c1c2C(NC(=O)CO)CC3 ; CC[C@@]1(O)C(=O)OCc2c1cc1n(c2=O)Cc2c-1nc1cc(Cl)c(C)c3c1c2[C@H](NC(=O)CO)CC3 ; CC[C@@]1(O)C(=O)OCc2c1cc1n(c2=O)Cc2c-1nc1cc(Cl)c(C)c3c1c2[C@@H](NC(=O)CO)CC3] 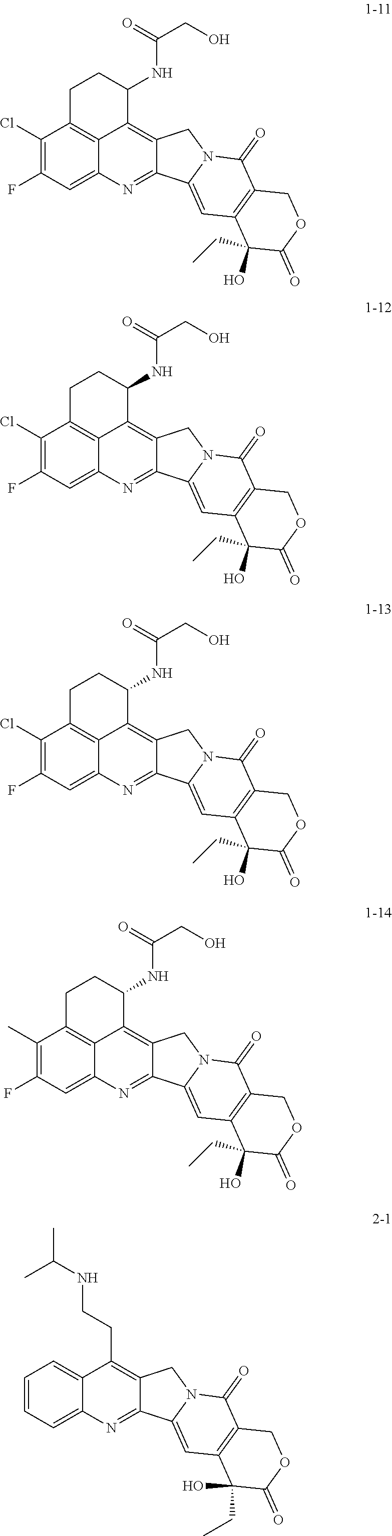 [SMILES: CC[C@@]1(O)C(=O)OCc2c1cc1n(c2=O)Cc2c-1nc1cc(F)c(Cl)c3c1c2C(NC(=O)CO)CC3 ; CC[C@@]1(O)C(=O)OCc2c1cc1n(c2=O)Cc2c-1nc1cc(F)c(Cl)c3c1c2[C@H](NC(=O)CO)CC3 ; CC[C@@]1(O)C(=O)OCc2c1cc1n(c2=O)Cc2c-1nc1cc(F)c(Cl)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)CO)CC3 ; CC[C@@]1(O)C(=O)OCc2c1cc1n(c2=O)Cc2c-1nc1ccccc1c2CCNC(C)C] 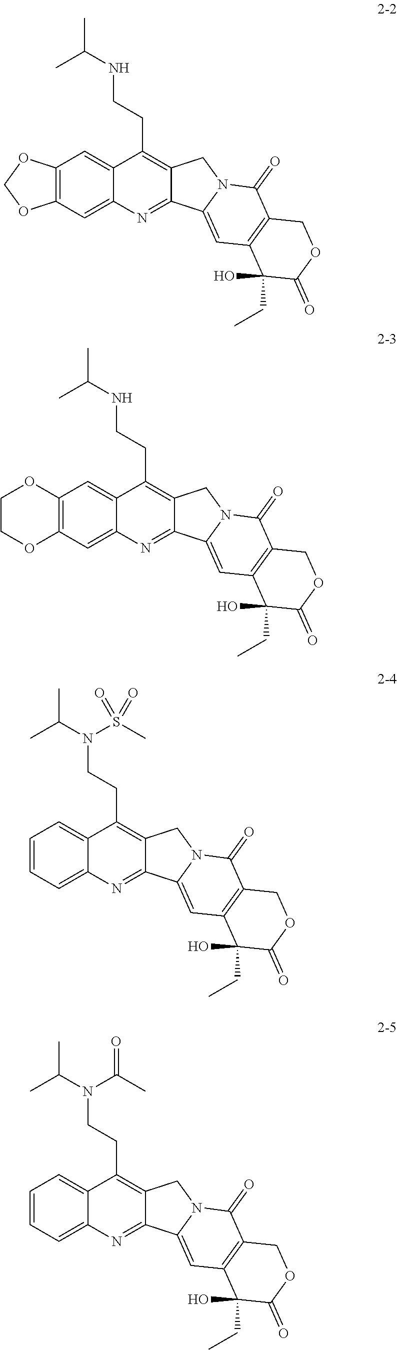 [SMILES: CC[C@@]1(O)C(=O)OCc2c1cc1n(c2=O)Cc2c-1nc1cc3c(cc1c2CCNC(C)C)OCO3 ; CC[C@@]1(O)C(=O)OCc2c1cc1n(c2=O)Cc2c-1nc1cc3c(cc1c2CCNC(C)C)OCCO3 ; CC[C@@]1(O)C(=O)OCc2c1cc1n(c2=O)Cc2c-1nc1ccccc1c2CCN(C(C)C)S(C)(=O)=O ; CC[C@@]1(O)C(=O)OCc2c1cc1n(c2=O)Cc2c-1nc1ccccc1c2CCN(C(C)=O)C(C)C] 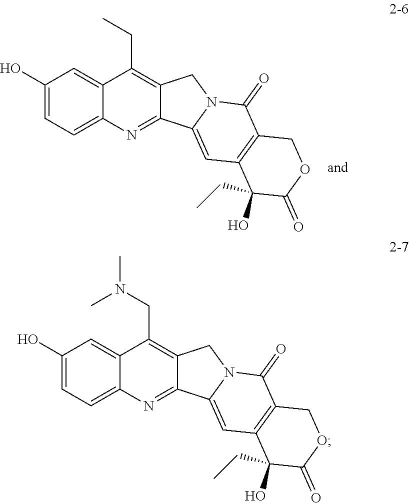 [SMILES: CCc1c2c(nc3ccc(O)cc13)-c1cc3c(c(=O)n1C2)COC(=O)[C@]3(O)CC ; CC[C@@]1(O)C(=O)OCc2c1cc1n(c2=O)Cc2c-1nc1ccc(O)cc1c2CN(C)C]
wherein the corresponding fragment of the cytotoxic drug obtained after the cytotoxic drug is linked to the linker is D as shown in the general formula; and
(III) the antibody that binds PTK7 is selected from the group consisting of:
(1) the following heavy chain variable regions (VH) and/or light chain variable regions (VL), wherein CDRs are defined according to a Chothia numbering system:
(1a) a heavy chain variable region (VH) comprising the following 3 CDRs: CDR-H1 having the amino acid sequence as set forth in SEQ ID NO: 11 or a variant thereof, CDR-H2 having the amino acid sequence as set forth in SEQ ID NO: 12 or a variant thereof, and CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof, or,
(1b) a heavy chain variable region (VH) comprising the following 3 CDRs: CDR-H1 having the amino acid sequence as set forth in SEQ ID NO: 27 or a variant thereof, CDR-H2 having the amino acid sequence as set forth in SEQ ID NO: 28 or a variant thereof, CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (1a) and (1b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids compared with the sequence from which the variants are derived;
or,
(2) the following heavy chain variable region (VH) and/or light chain variable region (VL), wherein the CDRs are defined according to the Kabat numbering system:
(2a) a heavy chain variable region (VH) comprising the following 3 CDRs: CDR-H1 having the amino acid sequence as set forth in SEQ ID NO: 17 or a variant thereof, CDR-H2 having the amino acid sequence as set forth in SEQ ID NO: 18 or 19 or a variant thereof, CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(2b) a heavy chain variable region (VH) comprising the following 3 CDRs: CDR-H1 having the amino acid sequence as set forth in SEQ ID NO: 33 or a variant thereof, CDR-H2 having the amino acid sequence as set forth in SEQ ID NO: 34 or 35 or a variant thereof, and CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (2a) and (2b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids compared with the sequence from which the variants are derived;
or,
(3) the following heavy chain variable regions (VH) and/or light chain variable regions (VL), wherein the CDRs are defined according to the IMGT numbering system:
(3a) a heavy chain variable region (VH) comprising the following 3 CDRs: CDR-H1 having the amino acid sequence as set forth in SEQ ID NO: 20 or a variant thereof, CDR-H2 having the amino acid sequence as set forth in SEQ ID NO: 21 or a variant thereof, and CDR-H3 having the amino acid sequence as set forth in SEQ ID NO: 22 or a variant thereof; and/or, a light chain variable region (VL) comprising the following 3 CDRs: CDR-L1 having the amino acid sequence as set forth in SEQ ID NO: 23 or a variant thereof, CDR-L2 having the amino acid sequence as set forth in SEQ ID NO: 24 or a variant thereof, and CDR-L3 having the amino acid sequence as set forth in SEQ ID NO: 16 or a variant thereof, or,
(3b) a heavy chain variable region (VH) comprising the following 3 CDRs: CDR-H1 having the amino acid sequence as set forth in SEQ ID NO: 36 or a variant thereof, CDR-H2 having the amino acid sequence as set forth in SEQ ID NO: 37 or a variant thereof, and CDR-H3 having the amino acid sequence as set forth in SEQ ID NO: 38 or a variant thereof; and/or, a light chain variable region (VL) comprising the following 3 CDRs: CDR-L1 having the amino acid sequence as set forth in SEQ ID NO: 39 or a variant thereof, CDR-L2 having the amino acid sequence as set forth in SEQ ID NO: 40 or a variant thereof, and CDR-L3 having the amino acid sequence as set forth in SEQ ID NO: 32 or a variant thereof;
where the variants in any one of (3a) and (3b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids compared with the sequence from which the variants are derived;
and,
(4) the following heavy chain variable regions (VH) and/or light chain variable regions (VL), wherein the CDRs are defined according to the AbM numbering system:
(4a) a heavy chain variable region (VH) comprising the following 3 CDRs: CDR-H1 having the amino acid sequence as set forth in SEQ ID NO: 25 or a variant thereof, CDR-H2 having the amino acid sequence as set forth in SEQ ID NO: 26 or a variant thereof, and CDR-H3 having the amino acid sequence as set forth in SEQ ID NO: 13 or a variant thereof; and/or, a light chain variable region (VL) comprising the following 3 CDRs: CDR-L1 having the amino acid sequence as set forth in SEQ ID NO: 14 or a variant thereof, CDR-L2 having the amino acid sequence as set forth in SEQ ID NO: 15 or a variant thereof, and CDR-L3 having the amino acid sequence as set forth in SEQ ID NO: 16 or a variant thereof; or,
(4b) a heavy chain variable region (VH) comprising the following 3 CDRs: CDR-H1 having the amino acid sequence as set forth in SEQ ID NO: 41 or a variant thereof, CDR-H2 having the amino acid sequence as set forth in SEQ ID NO: 42 or a variant thereof, and CDR-H3 having the amino acid sequence as set forth in SEQ ID NO: 29 or a variant thereof; and/or, a light chain variable region (VL) comprising the following 3 CDRs: CDR-L1 having the amino acid sequence as set forth in SEQ ID NO: 30 or a variant thereof, CDR-L2 having the amino acid sequence as set forth in SEQ ID NO: 31 or a variant thereof, and CDR-L3 having the amino acid sequence as set forth in SEQ ID NO: 32 or a variant thereof;
where the variants in any one of (4a) and (4b) have at least 70%, at least 80%, at least 85%, at least 90%, at least 91%, at least 92%, at least 93%, at least 94%, at least 95%, at least 96%, at least 97%, at least 98%, at least 99% or 100% sequence identity compared with the sequence from which the variants are derived, or are subjected to replacement, deletion or addition of one or several amino acids compared with the sequence from which the variants are derived;
or, the antibody that binds PTK7 is selected from the group consisting of
(a) a VH having the amino acid sequence as set forth in SEQ ID NO: 1 or a variant thereof, and/or, a VL having the amino acid sequence as set forth in SEQ ID NO: 2 or a variant thereof;
(b) a VH having the amino acid sequence as set forth in SEQ ID NO: 3 or a variant thereof, and/or, a VL having the amino acid sequence as set forth in SEQ ID NO: 4 or a variant thereof;
(c) a VH having the amino acid sequence as set forth in SEQ ID NO: 5 or a variant thereof, and/or, a VL having the amino acid sequence as set forth in SEQ ID NO: 6 or a variant thereof;
(d) a VH having the amino acid sequence as set forth in SEQ ID NO: 7 or a variant thereof, and/or, a VL having the amino acid sequence as set forth in SEQ ID NO: 8 or a variant thereof; and
(e) a VH having the amino acid sequence as set forth in SEQ ID NO: 9 or a variant thereof, and/or, a VL having the amino acid sequence as set forth in SEQ ID NO: 10 or a variant thereof;
wherein the variants have at least 70%, at least 80%, at least 85%, at least 90%, at least 91%, at least 92%, at least 93%, at least 94%, at least 95%, at least 96%, at least 97%, at least 98%, at least 99% or 100% sequence identity as compared to the sequence from which the variants are derived, or are subjected to replacement, deletion or addition of one or several amino acids as compared to the sequence from which the variants are derived;
or, the antibody that binds PTK7 is selected from the group consisting of:
(1) a heavy chain comprising a VH having the amino acid sequence as set forth in SEQ ID NO: 1 and a heavy chain constant region (CH) shown as SEQ ID NO: 43, and, a light chain comprising a VL having the amino acid sequence as set forth in SEQ ID NO: 2 and a light chain constant region (CL) having the amino acid sequence as set forth in SEQ ID NO: 44;
(2) a heavy chain comprising a VH having the amino acid sequence as set forth in SEQ ID NO: 3 and a heavy chain constant region (CH) having the amino acid sequence as set forth in SEQ ID NO: 43 or 45, and, a light chain comprising a VL having the amino acid sequence as set forth in SEQ ID NO: 4 and a light chain constant region (CL) having the amino acid sequence as set forth in SEQ ID NO: 44;
(3) a heavy chain comprising a VH having the amino acid sequence as set forth in SEQ ID NO: 5 and a heavy chain constant region (CH) having the amino acid sequence as set forth in SEQ ID NO: 43, and, a light chain comprising a VL having the amino acid sequence as set forth in SEQ ID NO: 6 and a light chain constant region (CL) having the amino acid sequence as set forth in SEQ ID NO: 44;
(4) a heavy chain comprising a VH having the amino acid sequence as set forth in SEQ ID NO: 7 and a heavy chain constant region (CH) having the amino acid sequence as set forth in SEQ ID NO: 43, and, a light chain comprising a VL having the amino acid sequence as set forth in SEQ ID NO: 8 and a light chain constant region (CL) having the amino acid sequence as set forth in SEQ ID NO: 44; and
(5) a heavy chain comprising a VH having the amino acid sequence as set forth in SEQ ID NO: 9 and a heavy chain constant region (CH) having the amino acid sequence as set forth in SEQ ID NO: 43, and, a light chain comprising a VL sequence having the amino acid sequence as set forth in SEQ ID NO: 10 and a light chain constant region (CL) having the amino acid sequence as set forth in SEQ ID NO: 44;
or, the antibody that binds PTK7 comprises: a heavy chain having the amino acid sequence as set forth in SEQ ID NO: 46 and a light chain having the amino acid sequence as set forth in SEQ ID NO: 47.

92. A pharmaceutical composition, comprising the antibody-drug conjugate according to claim 74, and one or more pharmaceutically acceptable excipients.

93. A method of treating a cancer in a subject with high expression of PTK7 comprising administering a therapeutic effective amount of the antibody-drug conjugate according to claim 74, or a pharmaceutical composition comprising the antibody-drug conjugate to the subject.
